(19) United States
(12) Patent Application Publication
(10) Pub. No.: US 2023/0035659 A1
(43) Pub. Date: Feb. 2, 2023

(54) Compositions and Methods for TTR Gene Editing and Treating ATTR Amyloidosis Comprising a Corticosteroid or Use Thereof

(71) Applicant: Intellia Therapeutics, Inc., Cambridge, MA (US)

(72) Inventors: Yong CHANG, Acton, MA (US); Seth C. ALEXANDER, Medford, MA (US); Kristy M. WOOD, Wellesley, MA (US); Arti Mahendra Prakash KANJOLIA, Malden, MA (US); Shobu ODATE, Arlington, MA (US); Jessica Lynn SEITZER, Windham, NH (US); Reynald Michael LESCARBEAU, Medford, MA (US); Walter STRAPPS, Dedham, MA (US)

(73) Assignee: Intellia Therapeutics, Inc., Cambridge, MA (US)

(21) Appl. No.: 17/486,758

(22) Filed: Sep. 27, 2021

Related U.S. Application Data
(63) Continuation of application No. PCT/US2020/025533, filed on Mar. 27, 2020.
(60) Provisional application No. 62/825,637, filed on Mar. 28, 2019.
(60) Provisional application No. 62/825,676, filed on Mar. 28, 2019.

(51) Int. Cl. A61K 48/00; A61K 31/573; A61K 45/06; C12N 15/90; C12N 15/88; C12N 9/22; A61P 25/00

(52) U.S. Cl. CPC A61K 48/005; A61K 31/573; A61K 45/06; C12N 15/907; C12N 15/88; B82Y 5/00; A61P 25/00; C12N 2310/20; C12N 2310/315; C12N 2320/31; C12N 9/22

(57) ABSTRACT

Compositions and methods for editing, e.g., introducing double-stranded breaks, within the TTR gene in combination with administration of a corticosteroid are provided. Compositions and methods for treating subjects having amyloidosis associated with transthyretin (ATTR), in which a guide RNA and a corticosteroid are administered, are provided.

This is a continuation application of International Application No. PCT/US2020/025533, filed on Mar. 27, 2020, which claims priority to U.S. Provisional Patent Application No. 62/825,676 filed Mar. 28, 2019 and U.S. Provisional Patent Application No. 62/825,637 filed Mar. 28, 2019, the contents of each of which are incorporated herein by reference in their entirety for all purposes.

The instant application contains a Sequence Listing which has been submitted electronically in ASCII format and is hereby incorporated by reference in its entirety. Said ASCII copy, created on Sep. 27, 2021, is named 2021-09-27_01155-0029-00US_ST25.txt and is 968 KB in size.

Transthyretin (TTR) is a protein produced by the TTR gene that normally functions to transport retinol and thyroxine throughout the body. TTR is predominantly synthesized in the liver, with small fractions being produced in the choroid plexus and retina. TTR normally circulates as a soluble tetrameric protein in the blood.

Pathogenic variants of TTR, which may disrupt tetramer stability, can be encoded by mutant alleles of the TTR gene. Mutant TTR may result in misfolded TTR, which may generate amyloids (i.e., aggregates of misfolded TTR protein). In some cases, pathogenic variants of TTR can lead to amyloidosis, or disease resulting from build-up of amyloids. For example, misfolded TTR monomers can polymerize into amyloid fibrils within tissues, such as the peripheral nerves, heart, and gastrointestinal tract. Amyloid plaques can also comprise wild-type TTR that has deposited on misfolded TTR.

Misfolding and deposition of wild-type TTR has also been observed in males aged 60 or more and is associated with heart rhythm problems, heart failure, and carpal tunnel.

Amyloidosis characterized by deposition of TTR may be referred to as “ATTR,” “TTR-related amyloidosis,” “TTR amyloidosis,” or “ATTR amyloidosis,” “ATTR familial amyloidosis” (when associated with a genetic mutation in a family), or “ATTRwt” or “wild-type ATTR” (when arising from misfolding and deposition of wild-type TTR).

ATTR can present with a wide spectrum of symptoms, and patients with different classes of ATTR may have different characteristics and prognoses. Some classes of ATTR include familial amyloid polyneuropathy (FAP), familial amyloid cardiomyopathy (FAC), and wild-type TTR amyloidosis (wt-TTR amyloidosis). FAP commonly presents with sensorimotor neuropathy, while FAC and wt-TTR amyloidosis commonly present with congestive heart failure. FAP and FAC are usually associated with a genetic mutation in the FIR gene, and more than 100 different mutations in the TTR gene have been associated with ATTR. In contrast, wt-TTR amyloidosis is associated with aging and not with a genetic mutation in TTR. It is estimated that approximately 50,000 patients worldwide may be affected by FAP and FAC.

While more than 100 mutations in TTR are associated with ATTR, certain mutations have been more closely associated with neuropathy and/or cardiomyopathy. For example, mutations at T60 of TTR are associated with both cardiomyopathy and neuropathy; mutations at V30 are more associated with neuropathy; and mutations at V122 are more associated with cardiomyopathy.

A range of treatment approaches have been studied for treatment of ATTR, but there are no approved drugs that stop disease progression and improve quality of life. While liver transplant has been studied for treatment of ATTR, its use is declining as it involves significant risk and disease progression sometimes continues after transplantation. Small molecule stabilizers, such as diflunisal and tafamidis, appear to slow ATTR progression, but these agents do not halt disease progression.

Approaches using small interfering RNA (siRNA) knockdown, antisense knockdown, or a monoclonal antibody targeting amyloid fibrils for destruction are also currently being investigated, but while results on short-term suppression of TTR expression show encouraging preliminary data, a need exists for treatments that can produce long-lasting suppression of TTR.

Administration of foreign RNA can cause innate immune responses which are undesirable in the context of gene editing and therapy. Accordingly, the present disclosure provides compositions and methods for gene editing that may reduce inflammation or immune responses. For example, coadministration of corticosteroids to subjects receiving guide RNAs may reduce such inflammation or immune responses.

Accordingly, the following embodiments are provided. In some embodiments, the present invention provides compositions and methods using a corticosteroid in combination with a guide RNA and optionally an RNA-guided DNA binding agent such as the CRISPR/Cas system to substantially reduce or knockout expression of the TTR gene, thereby substantially reducing or eliminating the production of TTR protein associated with ATTR. The substantial reduction or elimination of the production of TTR protein associated with ATTR through alteration of the TTR gene can be a long-term reduction or elimination.

### SUMMARY

The following embodiments are provided herein.

Embodiment 1 is a method of treating amyloidosis associated with TTR (ATTR), comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82, thereby treating ATTR.

Embodiment 2 is a method of reducing TTR serum concentration, comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,

thereby reducing TTR serum concentration.

Embodiment 3 is a method for reducing or preventing the accumulation of amyloids or amyloid fibrils comprising TTR in a subject, comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
   thereby reducing accumulation of amyloids or amyloid fibrils.

Embodiment 4 is a composition comprising a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
   for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

Embodiment 5 is a composition comprising a vector encoding a guide RNA, wherein the guide RNA comprises:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
   for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

Embodiment 6 is a composition comprising:

(i) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82, and
   (ii) an mRNA that encodes an RNA-guided DNA binding agent, wherein:
the open reading frame comprises a sequence with at least 95% identity to SEQ ID NO: 311;
the open reading frame has at least 95% identity to SEQ ID NO: 311 over at least its first 30, 50, 70, 100, 150, 200, 250, or 300 nucleotides;
the open reading frame consists of a set of codons of which at least 75% of the codons are codons listed in Table 1;
the open reading frame has an adenine content ranging from its minimum adenine content to 150% of the minimum adenine content; and/or
the open reading frame has an adenine dinucleotide content ranging from its minimum adenine dinucleotide content to 150% of the minimum adenine dinucleotide content;
   for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

Embodiment 7 is a composition comprising:

(i) a vector encoding a guide RNA, wherein the guide RNA comprises:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82, and
   (ii) an mRNA that encodes an RNA-guided DNA binding agent, wherein:

the open reading frame comprises a sequence with at least 95% identity to SEQ ID NO: 311;
the open reading frame has at least 95% identity to SEQ ID NO: 311 over at least its first 30, 50, 70, 100, 150, 200, 250, or 300 nucleotides;
the open reading frame consists of a set of codons of which at least 75% of the codons are codons listed in Table 1;
the open reading frame has an adenine content ranging from its minimum adenine content to 150% of the minimum adenine content; and/or
the open reading frame has an adenine dinucleotide content ranging from its minimum adenine dinucleotide content to 150% of the minimum adenine dinucleotide content;
   for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

Embodiment 8 is the composition for use or method of any one of embodiments 1-3 or 5-7, wherein the method comprises administering the composition by infusion for more than 30 minutes, e.g. more than 60 minutes or more than 120 minutes.

Embodiment 9 is the composition or method of any one of the preceding embodiments, wherein the guide RNA comprises a guide sequence selected from SEQ ID NOs: 5-72, 74-78, and 80-82.

Embodiment 10 is the composition or method of any one of the preceding embodiments, wherein the guide RNA comprises a guide sequence selected from SEQ ID NOs: 5, 6, 7, 8, 9, 12, 13, 14, 15, 16, 17, 22, 23, 27, 29, 30, 35, 36, 37, 38, 55, 61, 63, 65, 66, 68, or 69.

Embodiment 11 is the composition of any one of embodiments 4-10, for use in inducing a double-stranded break (DSB) within the TTR gene in a cell or subject.

Embodiment 12 is the composition of any one of embodiments 4-11, for use in modifying the TTR gene in a cell or subject.

Embodiment 13 is the composition of any one of embodiments 4-12, for use in treating amyloidosis associated with TTR (ATTR) in a subject.

Embodiment 14 is the composition of any one of embodiments 4-13, for use in reducing TTR serum concentration in a subject.

Embodiment 15 is the composition of any one of embodiments 4-14, for use in reducing or preventing the accumulation of amyloids or amyloid fibrils in a subject.

Embodiment 16 is the method or composition for use of any one of the preceding embodiments, wherein the corticosteroid is dexamethasone, betamethasone, prednisone, prednisolone, methylprednisolone, cortisone, hydrocortisone, triamcinolone, or ethamethasoneb.

Embodiment 17 is the method or composition for use of any one of the preceding embodiments, wherein the corticosteroid is dexamethasone.

Embodiment 18 is the method or composition for use of any one of the preceding embodiments, wherein the corticosteroid is administered before the composition.

Embodiment 19 is the method or composition for use of any one of the preceding embodiments, wherein the corticosteroid is administered after the composition.

Embodiment 20 is the method or composition for use of any one of the preceding embodiments, wherein the corticosteroid is administered simultaneously with the composition.

Embodiment 21 is the method or composition for use of any one of the preceding embodiments, wherein the corticosteroid is administered about 5 minutes to within about 168 hours before the composition is administered.

Embodiment 22 is the method or composition for use of any one of the preceding embodiments, wherein the corticosteroid is administered about 5 minutes to within about 168 hours after the composition is administered.

Embodiment 23 is the method or composition for use of any one of the preceding embodiments, wherein the corticosteroid is administered 5 minutes, 10 minutes, 15 minutes, 30 minutes, 1 hour, 2 hours, 3 hours, 4 hours, 6 hours, 12 hours, 18 hours, 1 day, 1.5 days, 2 days, 3 days, 4 days, 5 days, 6 days, or one week before the composition is administered.

Embodiment 24 is the method or composition for use of any one of the preceding embodiments, wherein the corticosteroid is administered 5 minutes, 10 minutes, 15 minutes, 30 minutes, 1 hour, 2 hours, 3 hours, 4 hours, 6 hours, 12 hours, 18 hours, 1 day, 1.5 days, 2 days, 3 days, 4 days, 5 days, 6 days, or one week after the composition is administered.

Embodiment 25 is the method or composition for use of any one of the preceding embodiments, wherein at least two doses of the corticosteroid are administered before or after the administration of the composition.

Embodiment 26 is the method or composition for use of any one of the preceding embodiments, wherein at least two doses of the corticosteroid and at least two doses of the composition are administered.

Embodiment 27 is the method or composition for use of any one of the preceding embodiments, wherein the corticosteroid is administered to the subject at a dose of 0.75 mg to 20 mg.

Embodiment 28 is the method or composition for use of embodiment 27, wherein the corticosteroid is administered to the subject at a dose of about 0.01-0.4 mg/kg, such as 0.1-0.35 mg/kg or 0.25-0.35 mg/kg.

Embodiment 29 is the method or composition for use of any one of the preceding embodiments, wherein the corticosteroid is administered to the subject parenterally or by injection.

Embodiment 30 is the method or composition for use of any one of the preceding embodiments, wherein the corticosteroid is administered to the subject via an intravenous injection.

Embodiment 31 is the method or composition for use of any one of the preceding embodiments, wherein the corticosteroid is administered to the subject intramuscularly or by infusion.

Embodiment 32 is the method or composition for use of any one of embodiments 1-31, wherein the corticosteroid is administered to the subject orally.

Embodiment 33 is the method or composition for use of any one of embodiment 32, wherein the corticosteroid is administered to the subject orally before the composition is administered to the subject by intravenous injection.

Embodiment 34 is the method or composition for use of any one of embodiment 32, wherein the corticosteroid is administered to the subject orally after the composition is administered to the subject by intravenous injection.

Embodiment 35 is the method or composition for use of any one of embodiments 32 and 33, wherein the corticosteroid is dexamethasone, and the dexamethasone is administered to the subject orally in the amount of 20 mg 6 to 12 hour before the composition is administered to the subject.

Embodiment 36 is the method or composition for use of any one of embodiments 32, 33 or 35, wherein the corticosteroid is dexamethasone, and the dexamethasone is administered to the subject intravenously in the amount of 20 mg for 30 minutes 6 to 12 hour before the composition is administered to the subject.

Embodiment 37 is the method or composition for use of any one of the preceding embodiments, wherein the composition is administered by infusion for about 45-75 minutes, 75-105 minutes, 105-135 minutes, 135-165 minutes, 165-195 minutes, 195-225 minutes, 225-255 minutes, 255-285 minutes, 285-315 minutes, 315-345 minutes, or 345-375 minutes. In some embodiments, the composition is administered by infusion for about 1.5-6 hours.

Embodiment 38 is the method or composition for use of any one of the preceding embodiments, wherein the composition is administered by infusion for about 60 minutes, about 90 minutes, about 120 minutes, about 150 minutes, about 180 minutes, or about 240 minutes.

Embodiment 39 is the method or composition for use of any one of the preceding embodiments, wherein the composition is administered by infusion for about 120 minutes.

Embodiment 40 is the method or composition for use of any one of the preceding embodiments, wherein the corticosteroid is dexamethasone.

Embodiment 41 is the method or composition for use of any one of the preceding embodiments, wherein the method further comprises administering an infusion prophylaxis, wherein the infusion prophylaxis comprises one or more of acetaminophen, an H1 blocker, or an H2 blocker, optionally wherein the one or more of the acetaminophen, H1 blocker, or H2 blocker are concurrently administered with the corticosteroid and/or before the composition.

Embodiment 42 is the method or composition for use of embodiment 41, wherein each of the acetaminophen, H1 blocker, and H2 blocker are administered.

Embodiment 42a is the method or composition for use of embodiment 41 or 42, wherein the H1 blocker and/or the H2 blocker are administered orally.

Embodiment 42b is the method or composition for use of any one of embodiments 41-42a, wherein the infusion prophylaxis comprises an intravenous corticosteroid (such as dexamethasone 8-12 mg, or 10 mg or equivalent) and acetaminophen (such as oral acetaminophen 500 mg).

Embodiment 42c is the method or composition for use of any one of embodiments 41-42b, wherein the infusion prophylaxis is administered as a required premedication prior to administering a guide RNA-containing composition, e.g. an LNP composition.

Embodiment 43 is the method or composition for use of any one of embodiments 41-42c, wherein the H1 blocker is diphenhydramine.

Embodiment 44 is the method or composition for use of any one of embodiments 41-43, wherein the H2 blocker is ranitidine.

Embodiment 45 is the method or composition for use of any one of the preceding embodiments, wherein a first dose of the corticosteroid is administered at about 8-24 hours before the composition is administered and a second dose of the corticosteroid is administered at about 1-2 hours before the composition is administered.

Embodiment 46 is the method or composition for use of any one of the preceding embodiments, wherein a first dose of the corticosteroid is administered orally and a second dose of the corticosteroid is administered intravenously before the composition is administered.

Embodiment 47 is the method or composition for use of any one of embodiments 45 and 46, wherein the method further comprises administering one or more of acetaminophen, an H1 blocker, or an H2 blocker, optionally wherein the one or more of the acetaminophen, H1 blocker, or H2 blocker are concurrently administered with the second dose of the corticosteroid.

Embodiment 48 is the method or composition for use of any one of the preceding embodiments, wherein a first dose of the corticosteroid is administered orally at about 8-24 hours before the composition is administered and a second dose of the corticosteroid is administered intravenously at about 1-2 hours before the composition is administered.

Embodiment 49 is the method or composition for use of any one of the preceding embodiments, wherein a first dose of the corticosteroid is administered orally at about 8-24 hours before the composition is administered and a second dose of the corticosteroid is administered intravenously concurrently with administration of acetaminophen, H1 blocker and H2 blocker at about 1-2 hours before the composition is administered.

Embodiment 50 is the method or composition for use of any one of the preceding embodiments, wherein the corticosteroid is dexamethasone, and a first dose of dexamethasone in the amount of about 6-10 mg is administered to the subject orally at about 8-24 hours before the composition is administered to the subject, and a second dose of dexamethasone in the amount of about 8-12 mg is intravenously administered to the subject concurrently with oral administration of acetaminophen and intravenous administration of an H1 blocker and an H2 blocker, at about 1-2 hours before the composition is administered to the subject, optionally wherein the H1 blocker is diphenhydramine and the H2 blocker is ranitidine, and/or optionally wherein the subject is human.

Embodiment 51 is the method or composition for use of any one of the preceding embodiments, wherein the corticosteroid is dexamethasone, and a first dose of dexamethasone in the amount of 8 mg is administered to the subject orally at about 8-24 hours before the composition is administered to the subject, and a second dose of dexamethasone in the amount of 10 mg is intravenously administered to the subject concurrently with oral administration of acetaminophen and intravenous administration of an H1 blocker and an H2 blocker, at about 1-2 hours before the composition is administered to the subject, optionally wherein the H1 blocker is diphenhydramine and the H2 blocker is ranitidine.

Embodiment 52 is the method or composition for use of any one of the preceding embodiments, wherein the composition is administered in the amount of 3 mg/kg by infusion for about 1.5-6 hours; a first dose of the corticosteroid is administered orally at about 8-24 hours before infusion of the composition; and a second dose of the corticosteroid is administered intravenously at about 1-2 hours before infusion of the composition.

Embodiment 53 is the method or composition for use of any one of the preceding embodiments, wherein administering the corticosteroid improves tolerability of the composition comprising the guide RNA.

Embodiment 54 is the method or composition for use of any one of the preceding embodiments, wherein administering the corticosteroid reduces the incidence or severity of one or more of inflammation, nausea, vomiting, elevated ALT concentration in blood, hyperthermia, and/or hyperalgesia in response to the composition comprising the guide RNA.

Embodiment 55 is the method or composition for use of any one of the preceding embodiments, wherein administering the corticosteroid reduces or inhibits production or activity of one or more interferons and/or inflammatory cytokines in response to the composition comprising the guide RNA.

Embodiment 56 is the method or composition for use of any one of the preceding embodiments, wherein the composition reduces serum TTR levels.

Embodiment 57 is the method or composition for use of embodiment 56, wherein the serum TTR levels are reduced by at least 50% as compared to serum TTR levels before administration of the composition.

Embodiment 58 is the method or composition for use of embodiment 56, wherein the serum TTR levels are reduced by 50-60%, 60-70%, 70-80%, 80-90%, 90-95%, 95-98%, 98-99%, or 99-100% as compared to serum TTR levels before administration of the composition.

Embodiment 59 is the method or composition for use of any one of the preceding embodiments, wherein the composition results in editing of the TTR gene.

Embodiment 60 is the method or composition for use of embodiment 59, wherein the editing is calculated as a percentage of the population that is edited (percent editing).

Embodiment 61 is the method or composition for use of embodiment 60, wherein the percent editing is between 30 and 99% of the population.

Embodiment 62 is the method or composition for use of embodiment 61, wherein the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.

Embodiment 63 is the method or composition for use of any one of the preceding embodiments, wherein the composition reduces amyloid deposition in at least one tissue.

Embodiment 64 is the method or composition for use of embodiment 63, wherein the at least one tissue comprises one or more of stomach, colon, sciatic nerve, or dorsal root ganglion.

Embodiment 65 is the method or composition for use of any one of embodiments 63 and 64, wherein amyloid deposition is measured 8 weeks after administration of the composition.

Embodiment 66 is the method or composition for use of any one of embodiments 63-65, wherein amyloid deposition is compared to a negative control or a level measured before administration of the composition.

Embodiment 67 is the method or composition for use of any one of embodiments 63-66, wherein amyloid deposition is measured in a biopsy sample and/or by immunostaining.

Embodiment 68 is the method or composition for use of any one of embodiments 63-67, wherein amyloid deposition is reduced by between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the amyloid deposition seen in a negative control.

Embodiment 69 is the method or composition for use of any one of embodiments 63-68, wherein amyloid deposition is reduced by between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the amyloid deposition seen before administration of the composition.

Embodiment 70 is the method or composition for use of any one of the preceding embodiments, wherein the composition is administered or delivered at least two times.

Embodiment 71 is the method or composition for use of embodiment 70, wherein the composition is administered or delivered at least three times.

Embodiment 72 is the method or composition for use of embodiment 70, wherein the composition is administered or delivered at least four times.

Embodiment 73 is the method or composition for use of embodiment 70, wherein the composition is administered or delivered up to five, six, seven, eight, nine, or ten times.

Embodiment 74 is the method or composition for use of any one of embodiments 70-73, wherein the administration or delivery occurs at an interval of 1, 2, 3, 4, 5, 6, 7, 8, 9, 10, 11, 12, 13, 14, or 15 days.

Embodiment 75 is the method or composition for use of any one of embodiments 70-73, wherein the administration or delivery occurs at an interval of 1, 2, 3, 4, 5, 6, 7, 8, 9, 10, 11, 12, 13, 14, or 15 weeks.

Embodiment 76 is the method or composition for use of any one of embodiments 70-73, wherein the administration or delivery occurs at an interval of 1, 2, 3, 4, 5, 6, 7, 8, 9, 10, 11, 12, 13, 14, or 15 months.

Embodiment 77 is the method or composition of any one of the preceding embodiments, wherein the guide sequence is selected from SEQ ID NOs: 5-82.

Embodiment 78 is the method or composition of any one of the preceding embodiments, wherein the guide RNA is at least partially complementary to a target sequence present in the human TTR gene.

Embodiment 79 is the method or composition of embodiment 78, wherein the target sequence is in exon 1, 2, 3, or 4 of the human TTR gene.

Embodiment 80 is the method or composition of embodiment 78, wherein the target sequence is in exon 1 of the human TTR gene.

Embodiment 81 is the method or composition of embodiment 78, wherein the target sequence is in exon 2 of the human TTR gene.

Embodiment 82 is the method or composition of embodiment 78, wherein the target sequence is in exon 3 of the human TTR gene.

Embodiment 83 is the method or composition of embodiment 78, wherein the target sequence is in exon 4 of the human TTR gene.

Embodiment 84 is the method or composition for use of any one of the preceding embodiments, wherein the guide sequence is complementary to a target sequence in the positive strand of TTR.

Embodiment 85 is the method or composition of any one of embodiments 1-83, wherein the guide sequence is complementary to a target sequence in the negative strand of TTR.

Embodiment 86 is the method or composition of any one of embodiments 1-83, wherein the first guide sequence is complementary to a first target sequence in the positive strand of the TTR gene, and wherein the composition further comprises a second guide sequence that is complementary to a second target sequence in the negative strand of the TTR gene.

Embodiment 87 is the method or composition of any one of the preceding embodiments, wherein the guide RNA is a dual guide (dgRNA).

Embodiment 88 is the method or composition of any one of embodiments 1-86, wherein the guide RNA is a single guide (sgRNA).

Embodiment 89 is the method or composition of embodiment 88, wherein the sgRNA comprises any one of the guide sequences of SEQ ID NOs: 5-82 and nucleotides 21-100 of SEQ ID NO: 3.

Embodiment 90 is the method or composition of any one of embodiments 88 and 89, wherein the sgRNA comprises a guide sequence that is at least 99%, 98%, 97%, 96%, 95%, 94%, 93%, 92%, 91%, or 90% identical to a sequence selected from SEQ ID Nos: 87-124.

Embodiment 91 is the method or composition of embodiment 88, wherein the sgRNA comprises a sequence selected from SEQ ID Nos: 87-124.

Embodiment 92 is the method or composition of any one of the preceding embodiments, wherein the guide RNA comprises at least one modification.

Embodiment 93 is the method or composition of embodiment 92, wherein the at least one modification includes a 2′-O-methyl (2′-O-Me) modified nucleotide.

Embodiment 94 is the method or composition of embodiment 92 or 93, wherein the at least one modification includes a phosphorothioate (PS) bond between nucleotides.

Embodiment 95 is the method or composition of any one of embodiments 92-94, wherein the at least one modification includes a 2′-fluoro (2′-F) modified nucleotide.

Embodiment 96 is the method or composition of any one of embodiments 92-95, wherein the at least one modification includes a 5′ end modification, a 3′ end modification, or 5′ and 3′ end modifications.

Embodiment 97 is the method or composition of any one of embodiments 92-96, wherein the at least one modification includes a modification at one or more of the first five nucleotides at the 5′ end.

Embodiment 98 is the method or composition of any one of embodiments 92-97, wherein the at least one modification includes a modification at one or more of the last five nucleotides at the 3′ end.

Embodiment 99 is the method or composition of any one of embodiments 92-98, wherein the at least one modification includes PS bonds between the first four nucleotides.

Embodiment 100 is the method or composition of any one of embodiments 92-99, wherein the at least one modification includes PS bonds between the last four nucleotides.

Embodiment 101 is the method or composition of any one of embodiments 92-100, wherein the at least one modification includes 2′-O-Me modified nucleotides at the first three nucleotides at the 5′ end.

Embodiment 102 is the method or composition of any one of embodiments 92-101, wherein the at least one modification includes 2′-O-Me modified nucleotides at the last three nucleotides at the 3′ end.

Embodiment 103 is the method or composition of any one of embodiments 92-102, wherein the guide RNA comprises the modified nucleotides of SEQ ID NO: 3.

Embodiment 104 is the method or composition of any one of the preceding embodiments, wherein the composition further comprises a pharmaceutically acceptable excipient.

Embodiment 105 is the method or composition of any one of the preceding embodiments, wherein the guide RNA is associated with a lipid nanoparticle (LNP).

Embodiment 106 is the method or composition of embodiment 105, wherein the LNP comprises an ionizable lipid.

Embodiment 107 is the method or composition of embodiment 106, wherein the LNP comprises a biodegradable ionizable lipid.

Embodiment 108 is the method or composition of any one of embodiments 105-017, wherein the LNP comprises an amine lipid, e.g., a CCD lipid.

Embodiment 109 is the method or composition of any one of embodiments 105-108, wherein the LNP comprises a helper lipid.

Embodiment 110 is the method or composition of any one of embodiments 105-109, wherein the LNP comprises a stealth lipid, optionally wherein:

(i) the LNP comprises a lipid component and the lipid component comprises: about 50-60 mol-% amine lipid such as Lipid A, about 8-10 mol-% neutral lipid; and about 2.5-4 mol-% stealth lipid (e.g., a PEG lipid), wherein the remainder of the lipid component is helper lipid, and wherein the N/P ratio of the LNP composition is about 6;
(ii) the LNP comprises about 50-60 mol-% amine lipid such as Lipid A; about 27-39.5 mol-% helper lipid; about 8-10 mol-% neutral lipid; and about 2.5-4 mol-% stealth lipid (e.g., a PEG lipid), wherein the N/P ratio of the LNP composition is about 5-7 (e.g., about 6);
(iii) the LNP comprises a lipid component and the lipid component comprises: about 50-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 3-10;
(iv) the LNP comprises a lipid component and the lipid component comprises: about 40-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 6;
(v) the LNP comprises a lipid component and the lipid component comprises: about 50-60 mol-% amine lipid such as Lipid A; about 5-15 mol-% neutral lipid; and about 1.5-10 mol-% Stealth lipid (e.g., a PEG lipid), wherein the remainder of the lipid component is helper lipid, and wherein the N/P ratio of the LNP composition is about 6;
(vi) the LNP comprises a lipid component and the lipid component comprises: about 40-60 mol-% amine lipid such as Lipid A; about 0-10 mol-% neutral lipid; and about 1.5-10 mol-% Stealth lipid (e.g., a PEG lipid), wherein the remainder of the lipid component is helper lipid, and wherein the N/P ratio of the LNP composition is about 3-10;
(vii) the LNP comprises a lipid component and the lipid component comprises: about 40-60 mol-% amine lipid such as Lipid A; less than about 1 mol-% neutral lipid; and about 1.5-10 mol-% Stealth lipid (e.g., a PEG lipid), wherein the remainder of the lipid component is helper lipid, and wherein the N/P ratio of the LNP composition is about 3-10; (viii) the LNP comprises a lipid component and the lipid component comprises: about 40-60 mol-% amine lipid such as Lipid A; and about 1.5-10 mol-% Stealth lipid (e.g., a PEG lipid), wherein the remainder of the lipid component is helper lipid, wherein the N/P ratio of the LNP composition is about 3-10, and wherein the LNP composition is essentially free of or free of neutral phospholipid; or
(ix) the LNP comprises a lipid component and the lipid component comprises: about 50-60 mol-% amine lipid such as Lipid A; about 8-10 mol-% neutral lipid; and about 2.5-4 mol-% Stealth lipid (e.g., a PEG lipid), wherein the remainder of the lipid component is helper lipid, and wherein the N/P ratio of the LNP composition is about 3-7.

Embodiment 111 is the method or composition of any one of embodiments 105-110, wherein the LNP comprises a neutral lipid.

Embodiment 112 is the method or composition of any one of embodiments 105-111, wherein the amine lipid is present at about 50 mol-%.

Embodiment 113 is the method or composition of any one of embodiments 105-112, wherein the neutral lipid is present at about 9 mol-%.

Embodiment 114 is the method or composition of any one of embodiments 105-113, wherein the stealth lipid is present at about 3 mol-%.

Embodiment 115 is the method or composition of any one of embodiments 105-114, wherein the helper lipid is present at about 38 mol-%.

Embodiment 116 is the method or composition of any one of embodiments 105-115, wherein the N/P ratio of the LNP composition is about 6.

Embodiment 117 is the method or composition of any one of embodiments 105-116, wherein the LNP comprises a lipid component and the lipid component comprises: about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the LNP composition is about 6.

Embodiment 118 is the method or composition of any one of embodiments 105-117, wherein the amine lipid is Lipid A.

Embodiment 119 is the method or composition of any one of embodiments 105-118, wherein the neutral lipid is DSPC.

Embodiment 120 is the method or composition of any one of embodiments 105-119, wherein the stealth lipid is PEG2k-DMG.

Embodiment 121 is the method or composition of any one of embodiments 105-120, wherein the helper lipid is cholesterol.

Embodiment 122 is the method or composition of any one of embodiments 105-121, wherein the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

Embodiment 123 is the method or composition of any one of the preceding embodiments, wherein the composition further comprises an RNA-guided DNA binding agent.

Embodiment 124 is the method or composition of any one of the preceding embodiments, wherein the composition further comprises a polynucleotide that encodes an RNA-guided DNA binding agent.

Embodiment 125 is the method or composition of embodiment 124, wherein the polynucleotide is an mRNA.

Embodiment 126 is the method or composition of any one of embodiments 123-125, wherein the RNA-guided DNA binding agent is a Cas cleavase.

Embodiment 127 is the method or composition of any one of embodiments 123-126, wherein the RNA-guided DNA binding agent is a Cas from a Type-II CRISPR/Cas system.

Embodiment 128 is the method or composition of any one of embodiments 123-127, wherein the RNA-guided DNA binding agent is a Cas9.

Embodiment 129 is the method or composition of embodiment 128, wherein the RNA-guided DNA binding agent is an S. pyogenes Cas9 nuclease.

Embodiment 130 is the method or composition of any one of embodiments 124-129, wherein the polynucleotide comprises an open reading frame encoding an RNA-guided DNA binding agent, wherein:
a. the open reading frame comprises a sequence with at least 95% identity to SEQ ID NO: 311;
b. the open reading frame has at least 95% identity to SEQ ID NO: 311 over at least its first 30, 50, 70, 100, 150, 200, 250, or 300 nucleotides;
c. the open reading frame consists of a set of codons of which at least 75% of the codons are codons listed in Table 4;
d. the open reading frame has an adenine content ranging from its minimum adenine content to 150% of the minimum adenine content; and/or
e. the open reading frame has an adenine dinucleotide content ranging from its minimum adenine dinucleotide content to 150% of the minimum adenine dinucleotide content.

Embodiment 131 is the composition or method of embodiment 130, wherein the open reading frame has at least 95% identity to SEQ ID NO: 311 over at least its first 10%, 12%, 15%, 20%, 25%, 30%, or 35% of its sequence.

Embodiment 132 is the composition or method of embodiment 130 or 131, wherein the open reading frame comprises a sequence with at least 95%, 96%, 97%, 98%, 99%, 99.5%, or 100% identity to SEQ ID NO: 311.

Embodiment 133 is the composition or method of any one of embodiments 130-132, wherein at least 75%, 80%, 85%, 90%, 95%, 98%, 99%, or 100% of the codons of the open reading frame are codons listed in Table 4.

Embodiment 134 is the composition or method of any one of embodiments 130-133, wherein the open reading frame has an adenine content ranging from its minimum adenine content to 101%, 102%, 103%, 105%, 110%, 115%, 120%, 125%, 130%, 135%, 140%, 145%, or 150% of the minimum adenine content.

Embodiment 135 is the composition or method of any one of embodiments 130-134, wherein the open reading frame has an adenine dinucleotide content ranging from its minimum adenine dinucleotide content to 101%, 102%, 103%, 105%, 110%, 115%, 120%, 125%, 130%, 135%, 140%, 145%, or 150% of the minimum adenine dinucleotide content.

Embodiment 136 is the composition or method of any one of embodiments 124-135, wherein the polynucleotide comprises a 5′ UTR with at least 90% identity to any one of SEQ ID NOs: 232, 234, 236, 238, 241, or 275-277.

Embodiment 137 is the composition or method of any one of embodiments 124-136, wherein the polynucleotide comprises a 3′ UTR with at least 90% identity to any one of SEQ ID NOs: 233, 235, 237, 239, or 240.

Embodiment 138 is the composition or method of any one of embodiments 124-137, wherein the polynucleotide comprises a 5′ UTR and a 3′ UTR from the same source.

Embodiment 139 is the composition or method of any one of embodiments 124-138, wherein the polynucleotide comprises a 5′ cap selected from Cap0, Cap1, and Cap2.

Embodiment 140 is the composition or method of any one of embodiments 124-139, wherein the open reading frame comprises a sequence with at least 95%, 96%, 97%, 98%, 99%, 99.5%, or 100% identity to SEQ ID NO: 311.

Embodiment 141 is the composition or method of any of embodiments 125-140, wherein at least 10% of the uridine in the mRNA is substituted with a modified uridine.

Embodiment 142 is the composition or method of embodiment 141, wherein the modified uridine is one or more of N1-methyl-pseudouridine, pseudouridine, 5-methoxyuridine, or 5-iodouridine.

Embodiment 143 is the composition or method of embodiment 141, wherein the modified uridine is one or both of N1-methyl-pseudouridine or 5-methoxyuridine.

Embodiment 144 is the composition or method of embodiment 141, wherein the modified uridine is N1-methyl-pseudouridine.

Embodiment 145 is the composition or method of embodiment 141, wherein the modified uridine is 5-methoxyuridine.

Embodiment 146 is the composition or method of any one of embodiments 141-145, wherein 15% to 45% of the uridine is substituted with the modified uridine.

Embodiment 147 is the composition or method of any one of embodiments 141-146, wherein at least 20% or at least 30% of the uridine is substituted with the modified uridine.

Embodiment 148 is the composition or method of embodiment 147, wherein at least 80% or at least 90% of the uridine is substituted with the modified uridine.

Embodiment 149 is the composition or method of embodiment 147, wherein 100% of the uridine is substituted with the modified uridine.

Embodiment 150 is the method or composition of any one of embodiments 123-149, wherein the RNA-guided DNA binding agent is modified.

Embodiment 151 is the method or composition of embodiment 150, wherein the modified RNA-guided DNA binding agent comprises a nuclear localization signal (NLS).

Embodiment 152 is the method or composition of any one of the preceding embodiments, wherein the composition is a pharmaceutical formulation and further comprises a pharmaceutically acceptable carrier.

Embodiment 153 is the method or composition for use of any one of the preceding embodiments, wherein the composition reduces or prevents amyloids or amyloid fibrils comprising TTR.

Embodiment 154 is the method or composition for use of embodiment 153, wherein the amyloids or amyloid fibrils are in the nerves, heart, or gastrointestinal track.

Embodiment 155 is the method or composition for use of any one of the preceding embodiments, wherein non-homologous ending joining (NHEJ) leads to a mutation during repair of a DSB in the TTR gene.

Embodiment 156 is the method or composition for use of embodiment 155, wherein NHEJ leads to a deletion or insertion of a nucleotide(s) during repair of a DSB in the TTR gene.

Embodiment 157 is the method or composition for use of embodiment 156, wherein the deletion or insertion of a nucleotide(s) induces a frame shift or nonsense mutation in the TTR gene.

Embodiment 158 is the method or composition for use of embodiment 155 or 156, wherein a frame shift or nonsense mutation is induced in the TTR gene of at least 50% of liver cells.

Embodiment 159 is the method or composition for use of embodiment 158, wherein a frame shift or nonsense mutation is induced in the TTR gene of 50%-60%, 60%-70%, 70% or 80%, 80%-90%, 90-95%, 95%-99%, or 99%-100% of liver cells.

Embodiment 160 is the method or composition for use of any one of embodiments 156-159, wherein a deletion or insertion of a nucleotide(s) occurs in the TTR gene at least 50-fold or more than in off-target sites.

Embodiment 161 is the method or composition for use of embodiment 160, wherein the deletion or insertion of a nucleotide(s) occurs in the TTR gene 50-fold to 150-fold, 150-fold to 500-fold, 500-fold to 1500-fold, 1500-fold to 5000-fold, 5000-fold to 15000-fold, 15000-fold to 30000-fold, or 30000-fold to 60000-fold more than in off-target sites.

Embodiment 162 is the method or composition for use of any one of embodiments 156-161, wherein the deletion or insertion of a nucleotide(s) occurs at less than or equal to 3, 2, 1, or 0 off-target site(s) in primary human hepatocytes, optionally wherein the off-target site(s) does (do) not occur in a protein coding region in the genome of the primary human hepatocytes.

Embodiment 163 is the method or composition for use of embodiment 162, wherein the deletion or insertion of a nucleotide(s) occurs at a number of off-target sites in primary human hepatocytes that is less than the number of off-target sites at which a deletion or insertion of a nucleotide(s) occurs in Cas9-overexpressing cells, optionally wherein the off-target site(s) does (do) not occur in a protein coding region in the genome of the primary human hepatocytes.

Embodiment 164 is the method or composition for use of embodiment 163, wherein the Cas9-overexpressing cells are HEK293 cells stably expressing Cas9.

Embodiment 165 is the method or composition for use of any one of embodiments 162-164, wherein the number of off-target sites in primary human hepatocytes is determined by analyzing genomic DNA from primary human hepatocytes transfected in vitro with Cas9 mRNA and the guide RNA, optionally wherein the off-target site(s) does (do) not occur in a protein coding region in the genome of the primary human hepatocytes.

Embodiment 166 is the method or composition for use of any one of embodiments 162-164, wherein the number of off-target sites in primary human hepatocytes is determined by an oligonucleotide insertion assay comprising analyzing genomic DNA from primary human hepatocytes transfected in vitro with Cas9 mRNA, the guide RNA, and a donor oligonucleotide, optionally wherein the off-target site(s) does (do) not occur in a protein coding region in the genome of the primary human hepatocytes.

Embodiment 167 is the method or composition of any one of the preceding embodiments, wherein the sequence of the guide RNA is:
a) SEQ ID NO: 92 or 104;
b) SEQ ID NO: 87, 89, 96, or 113;
c) SEQ ID NO: 100, 102, 106, 111, or 112; or
d) SEQ ID NO: 88, 90, 91, 93, 94, 95, 97, 101, 103, 108, or 109,

optionally wherein the guide RNA does not produce indels at off-target site(s) that occur in a protein coding region in the genome of primary human hepatocytes.

Embodiment 168 is the method or composition for use of any one of the preceding embodiments, wherein administering the composition reduces levels of TTR in the subject.

Embodiment 169 is the method or composition for use of embodiment 168, wherein the levels of TTR are reduced by at least 50%.

Embodiment 170 is the method or composition for use of embodiment 169, wherein the levels of TTR are reduced by 50%-60%, 60%-70%, 70% or 80%, 80%-90%, 90-95%, 95%-99%, or 99%-100%.

Embodiment 171 is the method or composition for use of embodiment 168 or 169, wherein the levels of TTR are measured in serum, plasma, blood, cerebral spinal fluid, or sputum.

Embodiment 172 is the method or composition for use of embodiment 168 or 169, wherein the levels of TTR are measured in liver, choroid plexus, and/or retina.

Embodiment 173 is the method or composition for use of any one of embodiments 168-172, wherein the levels of TTR are measured via enzyme-linked immunosorbent assay (ELISA).

Embodiment 174 is the method or composition for use of any one of the preceding embodiments, wherein the subject has ATTR.

Embodiment 175 is the method or composition for use of any one of the preceding embodiments, wherein the subject is human.

Embodiment 176 is the method or composition for use of embodiment 174 or 175, wherein the subject has ATTRwt.

Embodiment 177 is the method or composition for use of embodiment 174 or 175, wherein the subject has hereditary ATTR.

Embodiment 178 is the method or composition for use of any one of the preceding embodiments, wherein the subject has a family history of ATTR.

Embodiment 179 is the method or composition for use of any one of the preceding embodiments, wherein the subject has familial amyloid polyneuropathy.

Embodiment 180 is the method or composition for use of any one of the preceding embodiments, wherein the subject has only or predominantly nerve symptoms of ATTR.

Embodiment 181 is the method or composition for use of any one of embodiments 1-179, wherein the subject has familial amyloid cardiomyopathy.

Embodiment 182 is the method or composition for use of any one of embodiments 1-179 or 181, wherein the subject has only or predominantly cardiac symptoms of ATTR.

Embodiment 183 is the method or composition for use of any one of the preceding embodiments, wherein the subject expresses TTR having a V30 mutation.

Embodiment 184 is the method or composition for use of embodiment 183, wherein the V30 mutation is V30A, V30G, V30L, or V30M.

Embodiment 185 is the method or composition for use of embodiment any one of the preceding embodiments, wherein the subject expresses TTR having a T60 mutation.

Embodiment 186 is the method or composition for use of embodiment 185, wherein the T60 mutation is T60A.

Embodiment 187 is the method or composition for use of embodiment any one of the preceding embodiments, wherein the subject expresses TTR having a V122 mutation.

Embodiment 188 is the method or composition for use of embodiment 187, wherein the V122 mutation is V122A, V122I, or V122(−).

Embodiment 189 is the method or composition for use of any one of the preceding embodiments, wherein the subject expresses wild-type TTR.

Embodiment 190 is the method or composition for use of any one of embodiments 1-182 or 189, wherein the subject does not express TTR having a V30, T60, or V122 mutation.

Embodiment 191 is the method or composition for use of any one of embodiments 1-182 or 189-190, wherein the subject does not express TTR having a pathological mutation.

Embodiment 192 is the method or composition for use of any one of embodiments 190-192, wherein the subject is homozygous for wild-type TTR.

Embodiment 193 is the method or composition for use of any one of the preceding embodiments, wherein after administration the subject has an improvement, stabilization, or slowing of change in symptoms of sensorimotor neuropathy.

Embodiment 194 is the method or composition for use of embodiment 193, wherein the improvement, stabilization, or slowing of change in sensory neuropathy is measured using electromyogram, nerve conduction tests, or patient-reported outcomes.

Embodiment 195 is the method or composition for use of any one of the preceding embodiments, wherein the subject has an improvement, stabilization, or slowing of change in symptoms of congestive heart failure.

Embodiment 196 is the method or composition for use of embodiment 195, wherein the improvement, stabilization, or slowing of change in congestive heart failure is measured using cardiac biomarker tests, lung function tests, chest x-rays, or electrocardiography.

Embodiment 197 is the method or composition for use of any one of the preceding embodiments, wherein the composition or pharmaceutical formulation is administered via a viral vector.

Embodiment 198 is the method or composition for use of any one of the preceding embodiments, wherein the composition or pharmaceutical formulation is administered via lipid nanoparticles.

Embodiment 199 is the method or composition for use of any one of the preceding embodiments, wherein the subject is tested for specific mutations in the TTR gene before administering the composition or formulation.

Embodiment 200 is the method or composition of any one of embodiments 1-199, wherein the sequence selected from SEQ ID NOs: 5-82 is SEQ ID NO: 5.

Embodiment 201 is the method or composition of any one of embodiments 1-199, wherein the sequence selected from SEQ ID NOs: 5-82 is SEQ ID NO: 6.

Embodiment 202 is the method or composition of any one of embodiments 1-199, wherein the sequence selected from SEQ ID NOs: 5-82 is SEQ ID NO: 7.

Embodiment 203 is the method or composition of any one of embodiments 1-199, wherein the sequence selected from SEQ ID NOs: 5-82 is SEQ ID NO: 8.

Embodiment 204 is the method or composition of any one of embodiments 1-199, wherein the sequence selected from SEQ ID NOs: 5-82 is SEQ ID NO: 9.

Embodiment 205 is the method or composition of any one of embodiments 1-199, wherein the sequence selected from SEQ ID NOs: 5-82 is SEQ ID NO: 10.

Embodiment 206 is the method or composition of any one of embodiments 1-199, wherein the sequence selected from SEQ ID NOs: 5-82 is SEQ ID NO: 11.

Embodiment 207 is the method or composition of any one of embodiments 1-199, wherein the sequence selected from SEQ ID NOs: 5-82 is SEQ ID NO: 12.

Embodiment 208 is the method or composition of any one of embodiments 1-199, wherein the sequence selected from SEQ ID NOs: 5-82 is SEQ ID NO: 13.

Embodiment 209 is the method or composition of any one of embodiments 1-199, wherein the sequence selected from SEQ ID NOs: 5-82 is SEQ ID NO: 14.

Embodiment 210 is the method or composition of any one of embodiments 1-199, wherein the sequence selected from SEQ ID NOs: 5-82 is SEQ ID NO: 15.

Embodiment 211 is the method or composition of any one of embodiments 1-199, wherein the sequence selected from SEQ ID NOs: 5-82 is SEQ ID NO: 16.

Embodiment 212 is the method or composition of any one of embodiments 1-199, wherein the sequence selected from SEQ ID NOs: 5-82 is SEQ ID NO: 17.

Embodiment 213 is the method or composition of any one of embodiments 1-199, wherein the sequence selected from SEQ ID NOs: 5-82 is SEQ ID NO: 18.

Embodiment 214 is the method or composition of any one of embodiments 1-199, wherein the sequence selected from SEQ ID NOs: 5-82 is SEQ ID NO: 19.

Embodiment 215 is the method or composition of any one of embodiments 1-199, wherein the sequence selected from SEQ ID NOs: 5-82 is SEQ ID NO: 20.

Embodiment 216 is the method or composition of any one of embodiments 1-199, wherein the sequence selected from SEQ ID NOs: 5-82 is SEQ ID NO: 21.

Embodiment 217 is the method or composition of any one of embodiments 1-199, wherein the sequence selected from SEQ ID NOs: 5-82 is SEQ ID NO: 22.

Embodiment 218 is the method or composition of any one of embodiments 1-199, wherein the sequence selected from SEQ ID NOs: 5-82 is SEQ ID NO: 23.

Embodiment 219 is the method or composition of any one of embodiments 1-199, wherein the sequence selected from SEQ ID NOs: 5-82 is SEQ ID NO: 24.

Embodiment 220 is the method or composition of any one of embodiments 1-199, wherein the sequence selected from SEQ ID NOs: 5-82 is SEQ ID NO: 25.

Embodiment 221 is the method or composition of any one of embodiments 1-199, wherein the sequence selected from SEQ ID NOs: 5-82 is SEQ ID NO: 26.

Embodiment 222 is the method or composition of any one of embodiments 1-199, wherein the sequence selected from SEQ ID NOs: 5-82 is SEQ ID NO: 27.

Embodiment 223 is the method or composition of any one of embodiments 1-199, wherein the sequence selected from SEQ ID NOs: 5-82 is SEQ ID NO: 28.

Embodiment 224 is the method or composition of any one of embodiments 1-199, wherein the sequence selected from SEQ ID NOs: 5-82 is SEQ ID NO: 29.

Embodiment 225 is the method or composition of any one of embodiments 1-199, wherein the sequence selected from SEQ ID NOs: 5-82 is SEQ ID NO: 30.

Embodiment 226 is the method or composition of any one of embodiments 1-199, wherein the sequence selected from SEQ ID NOs: 5-82 is SEQ ID NO: 31.

Embodiment 227 is the method or composition of any one of embodiments 1-199, wherein the sequence selected from SEQ ID NOs: 5-82 is SEQ ID NO: 32.

Embodiment 228 is the method or composition of any one of embodiments 1-199, wherein the sequence selected from SEQ ID NOs: 5-82 is SEQ ID NO: 33.

Embodiment 229 is the method or composition of any one of embodiments 1-199, wherein the sequence selected from SEQ ID NOs: 5-82 is SEQ ID NO: 34.

Embodiment 230 is the method or composition of any one of embodiments 1-199, wherein the sequence selected from SEQ ID NOs: 5-82 is SEQ ID NO: 35.

Embodiment 231 is the method or composition of any one of embodiments 1-199, wherein the sequence selected from SEQ ID NOs: 5-82 is SEQ ID NO: 36.

Embodiment 232 is the method or composition of any one of embodiments 1-199, wherein the sequence selected from SEQ ID NOs: 5-82 is SEQ ID NO: 37.

Embodiment 233 is the method or composition of any one of embodiments 1-199, wherein the sequence selected from SEQ ID NOs: 5-82 is SEQ ID NO: 38.

Embodiment 234 is the method or composition of any one of embodiments 1-199, wherein the sequence selected from SEQ ID NOs: 5-82 is SEQ ID NO: 39.

Embodiment 235 is the method or composition of any one of embodiments 1-199, wherein the sequence selected from SEQ ID NOs: 5-82 is SEQ ID NO: 40.

Embodiment 236 is the method or composition of any one of embodiments 1-199, wherein the sequence selected from SEQ ID NOs: 5-82 is SEQ ID NO: 41.

Embodiment 237 is the method or composition of any one of embodiments 1-199, wherein the sequence selected from SEQ ID NOs: 5-82 is SEQ ID NO: 42.

Embodiment 238 is the method or composition of any one of embodiments 1-199, wherein the sequence selected from SEQ ID NOs: 5-82 is SEQ ID NO: 43.

Embodiment 239 is the method or composition of any one of embodiments 1-199, wherein the sequence selected from SEQ ID NOs: 5-82 is SEQ ID NO: 44.

Embodiment 240 is the method or composition of any one of embodiments 1-199, wherein the sequence selected from SEQ ID NOs: 5-82 is SEQ ID NO: 45.

Embodiment 241 is the method or composition of any one of embodiments 1-199, wherein the sequence selected from SEQ ID NOs: 5-82 is SEQ ID NO: 46.

Embodiment 242 is the method or composition of any one of embodiments 1-199, wherein the sequence selected from SEQ ID NOs: 5-82 is SEQ ID NO: 47.

Embodiment 243 is the method or composition of any one of embodiments 1-199, wherein the sequence selected from SEQ ID NOs: 5-82 is SEQ ID NO: 48.

Embodiment 244 is the method or composition of any one of embodiments 1-199, wherein the sequence selected from SEQ ID NOs: 5-82 is SEQ ID NO: 49.

Embodiment 245 is the method or composition of any one of embodiments 1-199, wherein the sequence selected from SEQ ID NOs: 5-82 is SEQ ID NO: 50.

Embodiment 246 is the method or composition of any one of embodiments 1-199, wherein the sequence selected from SEQ ID NOs: 5-82 is SEQ ID NO: 51.

Embodiment 247 is the method or composition of any one of embodiments 1-199, wherein the sequence selected from SEQ ID NOs: 5-82 is SEQ ID NO: 52.

Embodiment 248 is the method or composition of any one of embodiments 1-199, wherein the sequence selected from SEQ ID NOs: 5-82 is SEQ ID NO: 53.

Embodiment 249 is the method or composition of any one of embodiments 1-199, wherein the sequence selected from SEQ ID NOs: 5-82 is SEQ ID NO: 54.

Embodiment 250 is the method or composition of any one of embodiments 1-199, wherein the sequence selected from SEQ ID NOs: 5-82 is SEQ ID NO: 55.

Embodiment 251 is the method or composition of any one of embodiments 1-199, wherein the sequence selected from SEQ ID NOs: 5-82 is SEQ ID NO: 56.

Embodiment 252 is the method or composition of any one of embodiments 1-199, wherein the sequence selected from SEQ ID NOs: 5-82 is SEQ ID NO: 57.

Embodiment 253 is the method or composition of any one of embodiments 1-199, wherein the sequence selected from SEQ ID NOs: 5-82 is SEQ ID NO: 58.

Embodiment 254 is the method or composition of any one of embodiments 1-199, wherein the sequence selected from SEQ ID NOs: 5-82 is SEQ ID NO: 59.

Embodiment 255 is the method or composition of any one of embodiments 1-199, wherein the sequence selected from SEQ ID NOs: 5-82 is SEQ ID NO: 60.

Embodiment 256 is the method or composition of any one of embodiments 1-199, wherein the sequence selected from SEQ ID NOs: 5-82 is SEQ ID NO: 61.

Embodiment 257 is the method or composition of any one of embodiments 1-199, wherein the sequence selected from SEQ ID NOs: 5-82 is SEQ ID NO: 62.

Embodiment 258 is the method or composition of any one of embodiments 1-199, wherein the sequence selected from SEQ ID NOs: 5-82 is SEQ ID NO: 63.

Embodiment 259 is the method or composition of any one of embodiments 1-199, wherein the sequence selected from SEQ ID NOs: 5-82 is SEQ ID NO: 64.

Embodiment 260 is the method or composition of any one of embodiments 1-199, wherein the sequence selected from SEQ ID NOs: 5-82 is SEQ ID NO: 65.

Embodiment 261 is the method or composition of any one of embodiments 1-199, wherein the sequence selected from SEQ ID NOs: 5-82 is SEQ ID NO: 66.

Embodiment 262 is the method or composition of any one of embodiments 1-199, wherein the sequence selected from SEQ ID NOs: 5-82 is SEQ ID NO: 67.

Embodiment 263 is the method or composition of any one of embodiments 1-199, wherein the sequence selected from SEQ ID NOs: 5-82 is SEQ ID NO: 68.

Embodiment 264 is the method or composition of any one of embodiments 1-199, wherein the sequence selected from SEQ ID NOs: 5-82 is SEQ ID NO: 69.

Embodiment 265 is the method or composition of any one of embodiments 1-199, wherein the sequence selected from SEQ ID NOs: 5-82 is SEQ ID NO: 70.

Embodiment 266 is the method or composition of any one of embodiments 1-199, wherein the sequence selected from SEQ ID NOs: 5-82 is SEQ ID NO: 71.

Embodiment 267 is the method or composition of any one of embodiments 1-199, wherein the sequence selected from SEQ ID NOs: 5-82 is SEQ ID NO: 72.

Embodiment 268 is the method or composition of any one of embodiments 1-199, wherein the sequence selected from SEQ ID NOs: 5-82 is SEQ ID NO: 73.

Embodiment 269 is the method or composition of any one of embodiments 1-199, wherein the sequence selected from SEQ ID NOs: 5-82 is SEQ ID NO: 74.

Embodiment 270 is the method or composition of any one of embodiments 1-199, wherein the sequence selected from SEQ ID NOs: 5-82 is SEQ ID NO: 75.

Embodiment 271 is the method or composition of any one of embodiments 1-199, wherein the sequence selected from SEQ ID NOs: 5-82 is SEQ ID NO: 76.

Embodiment 272 is the method or composition of any one of embodiments 1-199, wherein the sequence selected from SEQ ID NOs: 5-82 is SEQ ID NO: 77.

Embodiment 273 is the method or composition of any one of embodiments 1-199, wherein the sequence selected from SEQ ID NOs: 5-82 is SEQ ID NO: 78.

Embodiment 274 is the method or composition of any one of embodiments 1-199, wherein the sequence selected from SEQ ID NOs: 5-82 is SEQ ID NO: 79.

Embodiment 275 is the method or composition of any one of embodiments 1-199, wherein the sequence selected from SEQ ID NOs: 5-82 is SEQ ID NO: 80.

Embodiment 276 is the method or composition of any one of embodiments 1-199, wherein the sequence selected from SEQ ID NOs: 5-82 is SEQ ID NO: 81.

Embodiment 277 is the method or composition of any one of embodiments 1-199, wherein the sequence selected from SEQ ID NOs: 5-82 is SEQ ID NO: 82.

Embodiment 278 is a use of a composition or formulation of any of the preceding embodiments for the preparation of a medicament for treating a human subject having ATTR.

### BRIEF DESCRIPTION OF THE DRAWINGS

FIG. 1 shows a schematic of chromosome 18 with the regions of the TTR gene that are targeted by the guide sequences provided in Table 1.

FIG. 2 shows off-target analysis in HEK293_Cas9 cells of certain dual guide RNAs targeting TTR. The on-target site is designated by a filled square for each dual guide RNA tested, whereas closed circles represent a potential off-target site.

FIG. 3 shows off-target analysis in HEK_Cas9 cells of certain single guide RNAs targeting TTR. The on-target site is designated by a filled square for each single guide RNA tested, whereas open circles represent a potential off-target site.

FIG. 4 shows dose response curves of lipid nanoparticle formulated human TTR specific sgRNAs on primary human hepatocytes.

FIG. 5 shows dose response curves of lipid nanoparticle formulated human TTR specific sgRNAs on primary cyno hepatocytes.

FIG. 6 shows dose response curves of lipid nanoparticle formulated cyno TTR specific sgRNAs on primary cyno hepatocytes.

FIG. 7 shows percent editing (% edit) of TTR and reduction of secreted TTR following administration of the guide in HUH7 cells sequences provided on the x-axis. The values are normalized to the amount of alpha-1-antitrypsin (AAT) protein.

FIG. 8 shows western blot analysis of intracellular TTR following administration of targeted guides (listed in Table 1) in HUH7 cells.

FIG. 9 shows percentage liver editing of TTR observed following administration of LNP formulations to mice with humanized (G481-G499) or murine (G282) TTR. Note: the first three ‘0’s in each Guide ID is omitted from the Figure, for example “G481” is “G000481” in Tables 2 and 3.

FIGS. 10A-B show serum TTR levels observed following the dosing regimens indicated on the horizontal axis as μg/ml (FIG. 10A) or percentage of TSS control (FIG. 10B). MPK=mg/kg throughout.

FIGS. 11A-B show serum TTR levels observed following the dosing regimens indicated on the horizontal axis for 1 mg/kg (FIG. 11A) or 0.5 mg/kg dosages (FIG. 11B). Data for a single 2 mg/kg dose is included as the right column in both panels.

FIGS. 12A-B show percentage liver editing observed following the dosing regimens indicated on the horizontal axis for 1 mg/kg (FIG. 12A) or 0.5 mg/kg dosages (FIG. 12B). FIG. 12C shows percentage liver editing observed following a single dose at 0.5, 1, or 2 mg/kg.

FIG. 13 shows percent liver editing observed following administration of LNP formulations to mice humanized with respect to the TTR gene. Note: the first three ‘0’s in each Guide ID is omitted from the Figure, for example “G481” is “G000481” in Tables 2 and 3.

FIGS. 14A-B show that there is correlation between liver editing (FIG. 14A) and serum human TTR levels (FIG. 14B) following administration of LNP formulations to mice humanized with respect to the TTR gene. Note: the first three ‘0’s in each Guide ID is omitted from the Figure, for example “G481” is “G000481” in Tables 2 and 3.

FIGS. 15A-B show that there is a dose response with respect to percent editing (FIG. 15A) and serum TTR levels (FIG. 15B) in wild type mice following administration of LNP formulations comprising guide G502, which is cross homologous between mouse and cyno.

FIG. 16 shows dose response curves of lipid nanoparticle formulated human TTR specific sgRNAs on primary cyno hepatocytes.

FIG. 17 shows dose response curves of lipid nanoparticle formulated cyno TTR specific sgRNAs on primary human hepatocytes.

FIG. 18 shows dose response curves of lipid nanoparticle formulated cyno TTR specific sgRNAs on primary cyno hepatocytes.

FIGS. 19A-D show serum TTR (% TSS; FIGS. 19A and 19C) and editing results following dosing of LNP formulations at the indicated ratios and amounts (FIGS. 19B and 19D).

FIG. 20 shows off-target analysis of certain single guide RNAs in Primary Human Hepatocytes (PHH) targeting TTR. In the graph, filled squares represent the identification of the on-target cut site, while open circles represent the identification of potential off-target sites.

FIGS. 21A-B show percent editing on-target (ONT, FIG. 21A) and at two off-target sites (OT2 and OT4) in primary human hepatocytes following administration of lipid nanoparticle formulated G000480. FIG. 21B is a re-scaled version of the OT2, OT4, and negative control (Neg Cont) data in FIG. 21A.

FIGS. 22A-B show percent editing on-target (ONT, FIG. 22A) and at an off-target site (OT4) in primary human hepatocytes following administration of lipid nanoparticle formulated G000486. FIG. 22B is a re-scaled version of the OT4 and negative control (Neg Cont) data in FIG. 22A.

FIGS. 23A-B show percent editing (FIG. 23A) and number of insertion and deletion events at the TTR locus (FIG. 23B). FIG. 23A shows percent editing at the TTR locus in control and treatment (dosed with lipid nanoparticle formulated TTR specific sgRNA) groups. FIG. 23B shows the number of insertion and deletion events at the TTR locus when editing was observed in the treatment group of FIG. 23A.

FIGS. 24A-B show TTR levels in circulating serum (FIG. 24A) and cerebrospinal fluid (CSF) (FIG. 24B), respectively, in μg/mL for control and treatment (dosed with lipid nanoparticle formulated TTR specific sgRNA) groups. Treatment resulted in >99% knockdown of TTR levels in serum.

FIGS. 25A-D show immunohistochemistry images with staining for TTR in stomach (FIG. 25A), colon (FIG. 25B), sciatic nerve (FIG. 25C), and dorsal root ganglion (DRG) (FIG. 25D) from control and treatment (dosed with lipid nanoparticle formulated TTR specific sgRNA) mice. At right, bar graphs show reduction in TTR staining 8 weeks after treatment in treated mice as measured by percent occupied area for each tissue type.

FIGS. 26A-C show liver TTR editing (FIG. 26A) and serum TTR results (in μg/mL (FIG. 26B) and as percentage of TSS-treated control (FIG. 26C)), respectively, from humanized TTR mice dosed with LNP formulations across a range of doses with guides G000480, G000488, G000489 and G000502 and containing Cas9 mRNA (SEQ ID NO: 1) in a 1:1 ratio by weight to the guide.

FIGS. 27A-C show liver TTR editing (FIG. 27A) and serum TTR results (in μg/mL (FIG. 27B) and as percentage of TSS-treated control (FIG. 27C)), respectively, from humanized TTR mice dosed with LNP formulations across a range of doses with guides G000481, G000482, G000486 and G000499 and containing Cas9 mRNA (SEQ ID NO: 1) in a 1:1 ratio by weight to the guide.

FIGS. 28A-C show liver TTR editing (FIG. 28A) and serum TTR results (in μg/mL (FIG. 28B) and as percentage of TSS-treated control (FIG. 28C)), respectively, from humanized TTR mice dosed with LNP formulations across a range of doses with guides G000480, G000481, G000486, G000499 and G000502 and containing Cas9 mRNA (SEQ ID NO: 1) in a 1:2 ratio by weight to the guide.

FIG. 29 shows relative expression of TTR mRNA in primary human hepatocytes (PHH) after treatment with LNPs comprising Cas9 mRNA and a gRNA as indicated, as compared to negative (untreated) controls.

FIG. 30 shows relative expression of TTR mRNA in primary human hepatocytes (PHH) after treatment with LNPs comprising Cas9 mRNA and a gRNA as indicated, as compared to negative (untreated) controls.

FIGS. 31A-C show serum TTR levels (FIG. 31A), liver TTR editing (FIG. 31B), and circulating ALT levels (FIG. 31C) in an in vivo study in nonhuman primates comparing 30′ administration of LNPs to a long dosing protocol.

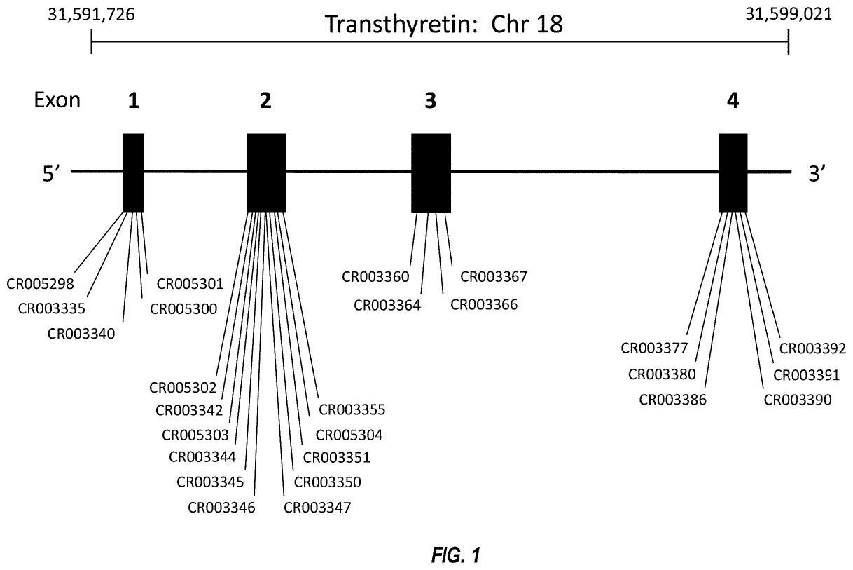

### DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs: 5-82. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 88%, 90%, 95%, 96%, 97%, 98%, 99%, or 100%. For example, in some embodiments, the guide sequence comprises a sequence with about 75%, 80%, 85%, 88%, 90%, 95%, 96%, 97%, 98%, 99%, or 100% identity to at least 17, 18, 19, or 20 contiguous nucleotides of a sequence selected from SEQ ID NOs: 5-82. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton, e.g., N1-methyl pseudouridine. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in, or accompanying, this disclosure can be a uridine or a modified uridine.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, the “minimum uridine content” of a given ORF is the uridine content of an ORF that (a) uses a minimal uridine codon at every position and (b) encodes the same amino acid sequence as the given ORF. The minimal uridine codon(s) for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating minimum uridine content.

As used herein, the “minimum uridine dinucleotide content” of a given ORF is the lowest possible uridine dinucleotide (UU) content of an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

As used herein, the “minimum adenine content” of a given open reading frame (ORF) is the adenine content of an ORF that (a) uses a minimal adenine codon at every position and (b) encodes the same amino acid sequence as the given ORF. The minimal adenine codon(s) for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating minimum adenine content.

As used herein, the “minimum adenine dinucleotide content” of a given open reading frame (ORF) is the lowest possible adenine dinucleotide (AA) content of an ORF that (a) uses a minimal adenine codon (as discussed above) at every position and (b) encodes the same amino acid sequence as the given ORF. The adenine dinucleotide (AA) content can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating minimum adenine dinucleotide content.

As used herein, “TTR” refers to transthyretin, which is the gene product of a TTR gene.

As used herein, “amyloid” refers to abnormal aggregates of proteins or peptides that are normally soluble. Amyloids are insoluble, and amyloids can create proteinaceous deposits in organs and tissues. Proteins or peptides in amyloids may be misfolded into a form that allows many copies of the protein to stick together to form fibrils. While some forms of amyloid may have normal functions in the human body, “amyloids” as used herein refers to abnormal or pathologic aggregates of protein. Amyloids may comprise a single protein or peptide, such as TTR, or they may comprise multiple proteins or peptides, such as TTR and additional proteins.

As used herein, “amyloid fibrils” refers to insoluble fibers of amyloid that are resistant to degradation. Amyloid fibrils can produce symptoms based on the specific protein or peptide and the tissue and cell type in which it has aggregated.

As used herein, “amyloidosis” refers to a disease characterized by symptoms caused by deposition of amyloid or amyloid fibrils. Amyloidosis can affect numerous organs including the heart, kidney, liver, spleen, nervous system, and digestive track.

As used herein, “ATTR,” “TTR-related amyloidosis,” “TTR amyloidosis,” “ATTR amyloidosis,” or “amyloidosis associated with TTR” refers to amyloidosis associated with deposition of TTR.

As used herein, “familial amyloid cardiomyopathy” or “FAC” refers to a hereditary transthyretin amyloidosis (ATTR) characterized primarily by restrictive cardiomyopathy. Congestive heart failure is common in FAC. Average age of onset is approximately 60-70 years of age, with an estimated life expectancy of 4-5 years after diagnosis.

As used herein, “familial amyloid polyneuropathy” or “FAP” refers to a hereditary transthyretin amyloidosis (ATTR) characterized primarily by sensorimotor neuropathy. Autonomic neuropathy is common in FAP. While neuropathy is a primary feature, symptoms of FAP may also include cachexia, renal failure, and cardiac disease. Average age of onset of FAP is approximately 30-50 years of age, with an estimated life expectancy of 5-15 after diagnosis.

As used herein, “wild-type ATTR” and “ATTRwt” refer to ATTR not associated with a pathological TTR mutation such as T60A, V30M, V30A, V30G, V30L, V122I, V122A, or V122(−). ATTRwt has also been referred to as senile systemic amyloidosis. Onset typically occurs in men aged 60 or higher with the most common symptoms being congestive heart failure and abnormal heart rhythm such as atrial fibrillation. Additional symptoms include consequences of poor heart function such as shortness of breath, fatigue, dizziness, swelling (especially in the legs), nausea, angina, disrupted sleep, and weight loss. A history of carpal tunnel syndrome indicates increased risk for ATTRwt and may in some cases be indicative of early-stage disease. ATTRwt generally leads to decreasing heart function over time but can have a better prognosis than hereditary ATTR because wild-type TTR deposits accumulate more slowly. Existing treatments are similar to other forms of ATTR (other than liver transplantation) and are generally directed to supporting or improving heart function, ranging from diuretics and limited fluid and salt intake to anticoagulants, and in severe cases, heart transplants. Nonetheless, like FAC, ATTRwt can result in death from heart failure, sometimes within 3-5 years of diagnosis.

Guide sequences useful in the guide RNA compositions and methods described herein are shown in Table 1 and throughout the application.

As used herein, “hereditary ATTR” refers to ATTR that is associated with a mutation in the sequence of the TTR gene. Known mutations in the TTR gene associated with ATTR include those resulting in TTR with substitutions of T60A, V30M, V30A, V30G, V30L, V122I, V122A, or V122(−).

As used herein, “indels” refer to insertion/deletion mutations consisting of a number of nucleotides that are either inserted or deleted at the site of double-stranded breaks (DSBs) in a target nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known, and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, methods are provided to “knockout” TTR in one or more cells (e.g., in a population of cells including in vivo populations such as those found in tissues). In some embodiments, a knockout is not the formation of mutant TTR protein, for example, created by indels, but rather the complete loss of expression of TTR protein in a cell.

As used herein, “mutant TTR” refers to a gene product of TTR (i.e., the TTR protein) having a change in the amino acid sequence of TTR compared to the wildtype amino acid sequence of TTR. The human wild-type TTR sequence is available at NCBI Gene ID: 7276; Ensembl: Ensembl: ENSG00000118271. Mutants forms of TTR associated with ATTR, e.g., in humans, include T60A, V30M, V30A, V30G, V30L, V122I, V122A, or V122(−).

As used herein, “mutant TTR” or “mutant TTR allele” refers to a TTR sequence having a change in the nucleotide sequence of TTR compared to the wildtype sequence (NCBI Gene ID: 7276; Ensembl: ENSG00000118271).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of ATTR may comprise alleviating symptoms of ATTR.

As used herein, the term “pathological mutation” refers to a mutation that renders a gene product, such as TTR, more likely to cause, promote, contribute to, or fail to inhibit the development of a disease, such as ATTR.

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1 and WO2019067992A1, the contents of which are hereby incorporated by reference in their entirety. Any LNP known to those of skill in the art to be capable of delivering nucleotides to subjects may be utilized with the guide RNAs and the nucleic acid encoding an RNA-guided DNA binding agent described herein.

As used herein, the terms “donor oligonucleotide” or “donor template” refers to a oligonucleotide that includes a desired nucleic acid sequence to be inserted into a target site (e.g., a target sit of a genomic DNA). A donor oligonucleotide may be a single-strand oligonucleotide or a double-strand oligonucleotide. In some embodiments, a donor oligonucleotide may be delivered with a guide RNA and a nucleic acid sequence encoding an RNA-guided DNA binding agent (e.g., Cas9) via use of LNP or transfection.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be linked to the remaining parts of the molecule by covalent bonds, hydrogen bonds or ionic interactions.

As used herein, the phrase “pharmaceutically acceptable” means that which is useful in preparing a pharmaceutical composition that is generally non-toxic and is not biologically undesirable and that are not otherwise unacceptable for pharmaceutical use.

The term “about” or “approximately” means an acceptable error for a particular value as determined by one of ordinary skill in the art, which depends in part on how the value is measured or determined.

As used herein, “infusion” refers to an active administration of one or more agents with an infusion time of, for example, between approximately 30 minutes and 12 hours. In some embodiments, the one or more agents comprise an LNP, e.g., comprising an mRNA encoding an RNA-guided DNA binding agent (such as Cas9) described herein and a gRNA described herein.

As used herein, “infusion prophylaxis” refers to a regimen administered to a subject before treatment (e.g., comprising administration of an LNP) comprising one or more, or all, of an intravenous corticosteroid (e.g., dexamethasone 10 mg or equivalent), an antipyretic (e.g. oral acetaminophen or paracetamol 500 mg), an intravenous H1 blocker (e.g., diphenhydramine 50 mg or equivalent), and an intravenous H2 blocker (e.g., ranitidine 50 mg or equivalent). Infusion prophylaxis is optionally combined with advance administration of an oral corticosteroid (e.g., dexamethasone 8 mg or equivalent). In some embodiments, the oral corticosteroid is administered 8-24 hours prior to treatment. In some embodiments, one or more, or all, of an intravenous corticosteroid (e.g., dexamethasone 10 mg or equivalent), oral acetaminophen 500 mg, an intravenous H1 blocker (e.g., diphenhydramine 50 mg or equivalent), an intravenous H2 blocker (e.g., ranitidine 50 mg or equivalent) are administered 1-2 hours before treatment. In some embodiments, an H1 blocker and/or an H2 blocker are administered orally.

### II. Methods and Compositions Targeting the TTR Gene

Disclosed herein are methods for treating amyloidosis associated with TTR (ATTR) in a subject, reducing TTR serum concentration in a subject, and/or reducing or preventing the accumulation of amyloids or amyloid fibrils in a subject, and related compositions, including compositions for use in such methods. A corticosteroid, guide RNA, RNA-guided DNA binding agent, or polynucleotide encoding an RNA-guided DNA binding agent, such as any of those described herein, is also provided for use in a method disclosed herein. For example, in some embodiments, the disclosed compositions such as LNP compositions comprise a guide RNA targeting TTR and, optionally, an RNA-guided DNA binding agent or a nucleic acid comprising an open reading frame encoding such an RNA-guided DNA binding agent (e.g., a CRISPR/Cas system). The subjects treated with such methods and compositions may have wild-type or non-wild type TTR gene sequences, such as, for example, subjects with ATTR, which may be ATTR wt or a hereditary or familial form of ATTR.

The dosage, frequency and mode of administration of the corticosteroid, infusion prophylaxis, and the guide-RNA containing composition described herein can be controlled independently.

In some embodiments, the corticosteroid is administered before the guide RNA-containing composition described herein. In some embodiments, the corticosteroid is administered after the guide RNA-containing composition described herein. In some embodiments, the corticosteroid is administered simultaneously with the guide RNA-containing composition described herein. In some embodiments, multiple doses of the corticosteroid are administered before or after the administration of the guide RNA-containing composition. In some embodiments, multiple doses of the guide RNA-containing composition are administered before or after the administration of the corticosteroid. In some embodiments, multiple doses of the corticosteroid and multiple doses of the guide RNA-containing composition are administered.

The guide RNA-containing composition, e.g. an LNP composition comprising a guide RNA and optionally a polynucleotide encoding an RNA-guided DNA binding agent, may be administered by infusion. In some embodiments, the composition is administered by infusion for longer than 30 minutes. In some embodiments, the composition is administered by 30 minute infusion. In some embodiments, the composition is administered by infusion for longer than 60 minutes. In some embodiments, the composition is administered by infusion for longer than 90 minutes. In some embodiments, the composition is administered by infusion for longer than 120 minutes, longer than 150 minutes, longer than 180 minutes, longer than 240 minutes, longer than 300 minutes, or longer than 360 minutes. In some embodiments, the composition is administered by infusion for at least 1 hour, at least 2 hours, at least 4 hours, at least 6 hours, at least 7 hours, at least 8 hours, at least 9 hours, at least 10 hours, at least 11 hours or at least 12 hours. In some embodiments, the composition is administered by infusion for 0.5-1.5 hours, 1.5-2.5 hours, 2.5-3.5 hours, 3.5-4.5 hours, 4.5-5.5 hours, 5.5-6.5 hours, 6.5-7.5 hours, 7.5-8.5 hours, 8.5-9.5 hours, 9.5-10.5 hours, 10.5-11.5 hours, or 11.5-12.5 hours. In some embodiments, the composition is administered by infusion for about 60 minutes, about 90 minutes, about 120 minutes, about 150 minutes, about 180 minutes, about 240 minutes, about 300 minutes, or about 360 minutes. In some embodiments, the composition is administered by infusion for about 45-75 minutes, 75-105 minutes, 105-135 minutes, 135-165 minutes, 165-195 minutes, 195-225 minutes, 225-255 minutes, 255-285 minutes, 285-315 minutes, 315-345 minutes, or 345-375 minutes. In some embodiments, the composition is administered by infusion for about 1.5-6 hours.

In some embodiments, the corticosteroid is administered about 5 minutes to within about 168 hours before the administration of the guide RNA-containing composition described herein. In some embodiments, the corticosteroid is administered about 5 minutes to within about 168 hours after the administration of the guide RNA-containing composition described herein. In some embodiments, the corticosteroid is administered 5 minutes, 10 minutes, 15 minutes, 30 minutes, 1 hour, 2 hours, 3 hours, 4 hours, 6 hours, 12 hours, 18 hours, 24 hours, 36 hours, 48 hours, 72 hours, 96 hours, 120 hours, 144 hours, 168 hours, or an amount of time in a range bounded by any two of the preceding values before the administration of the guide RNA-containing composition described herein. In some embodiments, the corticosteroid is administered 1 hour, 2 hours, 3 hours, 4 hours, 6 hours, 12 hours, 18 hours, 24 hours, 36 hours, 48 hours, 72 hours, 96 hours, 120 hours, 144 hours 168 hours, or an amount of time in a range bounded by any two of the preceding values after the administration of the guide RNA-containing composition described herein. In certain embodiments, a corticosteroid is delivered about 8-24 hours before administration of the guide RNA-containing composition and an infusion prophylaxis is administered 1-2 hours prior to administration of the guide RNA-containing composition. The corticosteroid may be administered with or at about the same time as the administration of the guide RNA-containing composition described herein.

If appropriate, a dose of corticosteroid may be administered as at least two sub-doses administered separately at appropriate intervals. In some embodiments, the corticosteroid is administered at least two times before the administration of the guide RNA-containing composition described herein. In some embodiments, a dose of corticosteroid is administered at least two times after the administration of the guide RNA-containing composition described herein. In some embodiments, the corticosteroid is administered (e.g., before, with, and/or after the administration of the guide RNA-containing composition described herein) at an interval of 1 hour, 2 hours, 3 hours, 4 hours, 6 hours, 12 hours, 18 hours; 1, 2, 3, 4, 5, 6, 7, 8, 9, 10, 11, 12, 13, 14, or 15 days; 3, 4, 5, 6, 7, 8, 9, 10, 11, 12, 13, 14, or 15 weeks; or an amount of time in a range bounded by any two of the preceding values. In some embodiments, the corticosteroid is administered before the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values. In some embodiments, the corticosteroid is administered after the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values.

In some embodiments, the corticosteroid is administered at least two times. In some embodiments, the corticosteroid is administered is administered at least three times. In some embodiments, the corticosteroid is administered at least four times. In some embodiments, the corticosteroid is administered is up to five, six, seven, eight, nine, or ten times. A first dose may be oral and a second or subsequent dose may be by parenteral administration, e.g. infusion. Alternatively, a first dose may be parenteral and a second or subsequent dose may be by oral administration.

In some embodiments, the corticosteroid is administered orally before intravenous administration of a guide RNA-containing composition described herein. In some embodiments, the corticosteroid is administered orally at or after intravenous administration of a guide RNA-containing composition described herein.

A. Corticosteroid; Infusion Prophylaxis

The corticosteroid used in the disclosed methods and compositions is useful for treating subjects undergoing gene editing and/or therapy with gene editing compositions. Without wishing to be bound to any particular theory, corticosteroids may be useful for reducing inflammation or immune responses to foreign RNAs (guide RNA or mRNAs encoding RNA-guided DNA binding agent). The corticosteroid used in the disclosed methods and compositions may be any of those known in the art and/or commercially available from a number of sources.

In some embodiments, an infusion prophylaxis is administered to a subject before the gene editing composition, e.g., at a time 1-2 hours prior to the administration of the gene editing composition. In some embodiments, the infusion prophylaxis comprises one or more, or all, of an intravenous corticosteroid (e.g., dexamethasone 8-12 mg, such as 10 mg or equivalent, or any of the other corticosteroids described elsewhere herein), an antipyretic (e.g. oral acetaminophen (also called paracetamol) 500 mg), an H1 blocker (e.g., diphenhydramine 50 mg or equivalent), an H2 blocker (e.g., ranitidine 50 mg or equivalent). In some embodiments, the infusion prophylaxis comprises an intravenous corticosteroid (e.g., dexamethasone 8-12 mg, such as 10 mg or equivalent) and an antipyretic (e.g. oral acetaminophen or paracetamol 500 mg). In some embodiments, the H1 blocker (e.g., diphenhydramine 50 mg or equivalent) and/or H2 blocker (e.g., ranitidine 50 mg or equivalent) are administered orally. In some embodiments, the H1 blocker (e.g., diphenhydramine 50 mg or equivalent) and/or H2 blocker (e.g., ranitidine 50 mg or equivalent) are administered intravenously. In some embodiments an intravenous H1 blocker and/or an intravenous H2 blocker is substituted with an equivalent, e.g., an orally administered equivalent. Additionally or alternatively, an oral corticosteroid (e.g., dexamethasone 6-10 mg, such as 8 mg or equivalent, or any of the other corticosteroids described elsewhere herein) may be administered, e.g., 8-24 hours prior to treatment. These dosages may be used, e.g., when the subject is a human, e.g., an adult human. In some embodiments, the infusion prophylaxis consists of the following: an intravenous corticosteroid (e.g., dexamethasone 10 mg or equivalent) which may reduce the severity of inflammation, oral acetaminophen 500 mg which may reduce pain and fever and/or inhibit COX enzymes and/or prostaglandins, intravenous H1 blocker (e.g., diphenhydramine 50 mg or equivalent), and intravenous H2 blocker (e.g., ranitidine 50 mg, or equivalent) which act to block the action of histamine at the H1 and H2 receptors respectively, and may optionally be preceded by administration of oral dexamethasone (such as in the amount of 8 mg or equivalent), e.g., at 8-24 hours prior to the administration of the gene editing composition. The infusion prophylaxis may function to reduce adverse reactions associated with administering a guide RNA-containing composition, e.g. an LNP composition. In some embodiments, the corticosteroid and/or infusion prophylaxis is administered as a required premedication prior to administering a guide RNA-containing composition, e.g. an LNP composition.

In some embodiments, the corticosteroid is concurrently administered with one or more of acetaminophen, H1 blocker, or H2 blocker. In some embodiments, the corticosteroid is concurrently administered with acetaminophen and H1 blocker. In some embodiments, the the corticosteroid is concurrently administered with acetaminophen and H2 blocker. In some embodiments, the corticosteroid is concurrently administered with H1 blocker and H2 blocker. In some embodiments, an H1 blocker and/or an H2 blocker are administered orally. In some embodiments, the composition is concurrently administered with acetaminophen, H1 blocker, and H2 blocker.

Many H1 and H2 blockers are known in the art. In some embodiments, the H1 blocker is diphenhydramine, clemastine, cetirizine, terfenadine, doxylamine, mirtazapine, dexbrompheniramine, triprolidine, cyproheptadine, loratadine, hydroxyzine, cinnarizine, astemizole, azatadine, meclizine, carbinoxamine, epinastine, olopatadine, tripelennamine, brompheniramine, ketotifen, fexofenadine, desloratadine, azelastine, dimenhydrinate, promethazine, mequitazine, emedastine, levocabastine, chlorpheniramine, cyclizine, alimemazine, phenindamine, pheniramine, methapyrilene, flunarizine, mianserin, levocetirizine, esmirtazapine, mepyramine, alcaftadine, antazoline, chloropyramine, dimetindene, dimetotiazine, acrivastine, dexchlorpheniramine maleate, ebastine, mizolastine, gsk-1004723, oxatomide, dexchlorpheniramine, bepotastine, buclizine, risperidone, methdilazine, maprotiline, diphenylpyraline, bromodiphenhydramine, ziprasidone, olanzapine, clozapine, promazine, trazodone, doxepin, desipramine, orphenadrine, methotrimeprazine, clofedanol, chlorprothixene, quetiapine, asenapine, benzatropine, aripiprazole, amitriptyline, imipramine, nortriptyline, trimipramine, isothipendyl, chlorpromazine, iloperidone, zuclopenthixol, chlorcyclizine, amoxapine, butriptyline, cariprazine, bilastine, dosulepin, rupatadine, pizotifen, thonzylamine, benzquinamide, propiomazine, aceprometazine, aripiprazole lauroxil, or deptropine.

In some embodiments, the H2 blocker is ranitidine, nizatidine, cimetidine, or famotidine. Equivalent corticosteroids and dosages can be found, for example, in Liu et al., Allergy, Asthma & Clinical Immunology, 2013, 9:30. Equivalent antihistamines (H1 blockers and/or H2 blockers) and dosages include the customary dose for a suitable member of the class, as known in the art.

In some embodiments, at least two doses of the corticosteroid are administered before the administration of the composition. In some embodiments, a first dose of the corticosteroid is administered before a second dose of the corticosteroid is administered before the composition is administered. In some embodiments, a first dose of the corticosteroid is administered within 8-24 hours before the composition is administered. In some embodiments, a first dose of the corticosteroid is administered orally within 8-24 hours before the composition is administered. In some embodiments, a second dose of the corticosteroid is administered within 1-2 hours before the composition is administered. In some embodiments, a second dose of the corticosteroid is administered intravenously within 1-2 hours before the composition is administered. In some embodiments, a first dose of the corticosteroid is administered within 8-24 hours before the composition is administered and a second dose of the corticosteroid is administered within 1-2 hours before the composition is administered.

In some embodiments, a first dose of the corticosteroid is administered orally and a second dose of the corticosteroid is administered intravenously before the composition is administered. In some embodiments, a first dose of the corticosteroid is administered orally within 8-24 hours before the composition is administered and a second dose of the corticosteroid is administered intravenously within 1-2 hours before the composition is administered.

In some embodiments, a first dose of the corticosteroid is administered orally and a second dose of the corticosteroid is concurrently administered with one or more of acetaminophen, H1 blocker, or H2 blocker before the composition is administered. In some embodiments, a first dose of the corticosteroid is administered orally and a second dose of the corticosteroid is concurrently administered with acetaminophen, H1 blocker and H2 blocker before the composition is administered. In some embodiments, a first dose of the corticosteroid is administered orally within 8-24 hours before the composition is administered and a second dose of the corticosteroid is administered intravenously concurrently administered with one or more of acetaminophen, H1 blocker or H2 blocker within 1-2 hours before the composition is administered. In some embodiments, a first dose of the corticosteroid is administered orally within 8-24 hours before the composition is administered and a second dose of the corticosteroid is administered intravenously concurrently administered with acetaminophen, H1 blocker and H2 blocker within 1-2 hours before the composition is administered. In some embodiments, a first dose of the corticosteroid is administered orally within 8-24 hours before the composition is administered and a second dose of the corticosteroid is administered intravenously concurrently administered with acetaminophen, H1 blocker and H2 blocker within 1-2 hours before the composition is administered, wherein the acetaminophen is administered orally and the H1 blocker and H2 blocker are administered intravenously.

In some embodiments, administering the corticosteroid improves tolerability of the composition comprising the guide RNA. For example, compared to administration of the composition comprising the guide RNA without the corticosteroid, administering the corticosteroid may reduce the incidence or severity of one or more adverse effects, such as inflammation, nausea, vomiting, elevated ALT concentration in blood, hyperthermia, and/or hyperalgesia. In some embodiments, administering the corticosteroid reduces or inhibits production or activity of one or more interferons and/or inflammatory cytokines in response to the composition comprising the guide RNA.

Exemplary corticosteroids include, but are not limited to, dexamethasone, betamethasone, prednisone, prednisolone, methylprednisolone, cortisone, hydrocortisone, triamcinolone, or ethamethasone, or a pharmaceutically acceptable salt thereof. Exemplary corticosteroids include, but are not limited to, dexamethasone, betamethasone, prednisone (Rayos®, Horizon Pharma), prednisolone (Pred Forte®, Allergan; Omnipred™, Novartis) methylprednisolone (Medrol®, Pharmacia&Upjohn; Solu-Medrolx®, Pharmacia&Upjohn), cortisone, hydrocortisone, triamcinolone, ethamethasone, budesonide (ENTOCORT®, Perrigo Pharma Intl.; Rhinocort®, Symbicort®, Astrazeneca Pharms; Ulceris®, Valeant Pharms), paramethasone, and deflazacort. In some embodiments, the corticosteroid is dexamethasone.

The corticosteroid used in the disclosed methods may be administered according to regimens known in the art, e.g., US FDA-approved regimens. Suitable modes of administration include, but are not limited to, enteral, topical, and parenteral administration. The phrases “parenteral administration” and “administered parenterally” as used herein means modes of administration other than enteral (which includes oral) and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In some embodiments, the corticosteroid is administered to the subject parenterally or by injection. In some embodiments, the corticosteroid is administered to the subject by intravenous injection. In some embodiments, the corticosteroid is administered to the subject orally or enterally. In some embodiments, the corticosteroid is administered to the subject topically.

In some embodiments, e.g., comprising administration to or for use in a human subject, the corticosteroid can be administered in an amount that ranges from about 0.75 mg to about 25 mg. In some embodiments, e.g., comprising administration to or for use in a human subject, the corticosteroid can be administered in an amount that ranges from about 0.01-0.5 mg/kg, such as 0.1-0.40 mg/kg or 0.25-0.40 mg/kg.

In one example, dexamethasone is administered orally in the amount of 20 mg or 25 mg 6 to 12 hours before intravenous administration of the guide RNA. In another example, dexamethasone is administered intravenously in the amount of 20 mg or 25 mg for 30 minutes 6 to 12 hour before intravenous administration of the guide RNA. In another example, dexamethasone is administered orally in the amount of 8-12 mg, such as 10 mg, 8 to 24 hours before infusion of the guide RNA composition. In another example, dexamethasone is administered intravenously in the amount of 8-12 mg, such as 10 mg, 1-2 hour before infusion of the guide RNA composition. In another example, dexamethasone is administered orally in the amount of 8-12 mg, such as 10 mg, 8 to 24 hours before infusion of the guide RNA composition and dexamethasone is administered intravenously in the amount of 8-12 mg, such as 10 mg, 1-2 hour before infusion of the guide RNA composition.

In some embodiments, the corticosteroid is dexamethasone, and the dexamethasone is administered to the subject orally in the amount of 8 mg 8-24 hours before the composition is administered to the subject. In some embodiments, the corticosteroid is dexamethasone, and the dexamethasone is administered to the subject orally in the amount of 8 mg 8-24 hours before the composition is administered to the subject.

In some embodiments, the corticosteroid is dexamethasone, and the dexamethasone is administered to the subject intravenously in the amount of 10 mg 1-2 hours before the composition is administered to the subject. In some embodiments, the corticosteroid is dexamethasone, and the dexamethasone is administered to the subject intravenously in the amount of 10 mg 1-2 hours before the composition is administered to the subject.

In some embodiments, the corticosteroid is dexamethasone, and a first dose of dexamethasone in the amount of 8 mg is administered to the subject orally 8-24 hours before the composition is administered to the subject, and a second dose of dexamethasone in the amount of 10 mg is administered to the subject intravenously 1-2 hours before the composition is administered to the subject.

In some embodiments, the corticosteroid is dexamethasone, and a first dose of dexamethasone in the amount of 8 mg is administered to the subject orally 8-24 hours before the composition is administered to the subject, and a second dose of dexamethasone in the amount of 10 mg is administered to the subject intravenously 1-2 hours before the composition is administered to the subject, wherein the second dose of the corticosteroid is concurrently administered with one or more of acetaminophen, H1 blocker or H2 blocker.

In some embodiments, the corticosteroid is dexamethasone, and a first dose of dexamethasone in the amount of 8 mg is administered to the subject orally 8-24 hours before the composition is administered to the subject, and a second dose of dexamethasone in the amount of 10 mg is administered to the subject intravenously 1-2 hours before the composition is administered to the subject, wherein the second dose of the corticosteroid is concurrently administered with acetaminophen, H1 blocker and H2 blocker.

In some embodiments, the corticosteroid is dexamethasone, and a first dose of dexamethasone in the amount of 8 mg is administered to the subject orally 8-24 hours before the composition is administered to the subject, and a second dose of dexamethasone in the amount of 10 mg is administered to the subject intravenously, concurrently with oral administration of acetaminophen and intravenous administration of H1 blocker and H2 blocker, 1-2 hours before the composition is administered to the subject.

In some embodiments, the corticosteroid is dexamethasone, and a first dose of dexamethasone in the amount of 8 mg is administered to the subject orally 8-24 hours before the composition is administered to the subject, and a second dose of dexamethasone in the amount of 10 mg is administered to the subject intravenously, concurrently with oral administration of acetaminophen in the amount of 500 mg and intravenous administration of H1 blocker in the amount of 50 mg and H2 blocker in the amount of 50 mg, 1-2 hours before the composition is administered to the subject.

Further, it is recognized by those having ordinary skill in the art that the dose of corticosteroid is easily adjustable depending on the choice of particular corticosteroid. For example, for the purpose of comparison, the following are approximate equivalent mg dosages of corticosteroids: hydrocortisone 20 mg; cortisone 25 mg; prednisone or prednisolone 5 mg; deflazacort 6 mg; methylprednisolone 4 mg; dexamethasone or betamethasone 0.75 mg; triamcinolone 4 mg. Therefore, although the doses of corticosteroid presented in the above examples are based on dexamethasone, when another corticosteroid is to be administered to the patient, one of ordinary skill in the art would use the above conversion information to calculate equivalent doses of the other corticosteroid.

B. Guide RNA (gRNAs)

The guide RNA used in the disclosed methods and compositions comprises a guide sequence targeting the TTR gene. Exemplary guide sequences targeting the TTR gene are shown in Table 1 at SEQ ID Nos: 5-82.

TABLE 1
TTR targeted guide sequences, nomenclature, chromosomal coordinates, and
sequence.

| SEQ | | | | Chromosomal | |
|---|---|---|---|---|---|
| ID No. | Guide ID | Description | Species | Location | Guide Sequences* |
| 5 | CR003335 | TTR | Human | chr18:3159191 | CUGCUCCUCCUCUGCCUUGC |
| | | (Exon 1) | | 7-31591937 | |
| 6 | CR003336 | TTR | Human | chr18:3159192 | CCUCCUCUGCCUUGCUGGAC |
| | | (Exon 1) | | 2-31591942 | |
| 7 | CR003337 | TTR | Human | chr18:3159192 | CCAGUCCAGCAAGGCAGAGG |
| | | (Exon 1) | | 5-31591945 | |
| 8 | CR003338 | TTR | Human | chr18:3159192 | AUACCAGUCCAGCAAGGCAG |
| | | (Exon 1) | | 8-31591948 | |
| 9 | CR003339 | TTR | Human | chr18:3159193 | ACACAAAUACCAGUCCAGCA |
| | | (Exon 1) | | 4-31591954 | |
| 10 | CR003340 | TTR | Human | chr18:3159193 | UGGACUGGUAUUUGUGUCUG |
| | | (Exon 1) | | 7-31591957 | |
| 11 | CR003341 | TTR | Human | chr18:3159194 | CUGGUAUUUGUGUCUGAGGC |
| | | (Exon 1) | | 1-31591961 | |
| 12 | CR003342 | TTR | Human | chr18:3159288 | CUUCUCUACACCCAGGGCAC |
| | | (Exon 2) | | 0-31592900 | |
| 13 | CR003343 | TTR | Human | chr18:3159290 | CAGAGGACACUUGGAUUCAC |
| | | (Exon 2) | | 2-31592922 | |
| 14 | CR003344 | TTR | Human | chr18:3159291 | UUUGACCAUCAGAGGACACU |
| | | (Exon 2) | | 1-31592931 | |
| 15 | CR003345 | TTR | Human | chr18:3159291 | UCUAGAACUUUGACCAUCAG |
| | | (Exon 2) | | 9-31592939 | |
| 16 | CR003346 | TTR | Human | chr18:3159292 | AAAGUUCUAGAUGCUGUCCG |
| | | (Exon 2) | | 8-31592948 | |
| 17 | CR003347 | TTR | Human | chr18:3159294 | CAUUGAUGGCAGGACUGCCU |
| | | (Exon 2) | | 8-31592968 | |
| 18 | CR003348 | TTR | Human | chr18:3159294 | AGGCAGUCCUGCCAUCAAUG |
| | | (Exon 2) | | 8-31592968 | |
| 19 | CR003349 | TTR | Human | chr18:3159295 | UGCACGGCCACAUUGAUGGC |
| | | (Exon 2) | | 8-31592978 | |
| 20 | CR003350 | TTR | Human | chr18:3159296 | CACAUGCACGGCCACAUUGA |
| | | (Exon 2) | | 2-31592982 | |
| 21 | CR003351 | TTR | Human | chr18:3159297 | AGCCUUUCUGAACACAUGCA |
| | | (Exon 2) | | 4-31592994 | |
| 22 | CR003352 | TTR | Human | chr18:3159298 | GAAAGGCUGCUGAUGACACC |
| | | (Exon 2) | | 6-31593006 | |
| 23 | CR003353 | TTR | Human | chr18:3159298 | AAAGGCUGCUGAUGACACCU |
| | | (Exon 2) | | 7-31593007 | |
| 24 | CR003354 | TTR | Human | chr18:3159300 | ACCUGGGAGCCAUUUGCCUC |
| | | (Exon 2) | | 3-31593023 | |
| 25 | CR003355 | TTR | Human | chr18:3159300 | CCCAGAGGCAAAUGGCUCCC |
| | | (Exon 2) | | 7-31593027 | |
| 26 | CR003356 | TTR | Human | chr18:3159301 | GCAACUUACCCAGAGGCAAA |
| | | (Exon 2) | | 5-31593035 | |
| 27 | CR003357 | TTR | Human | chr18:3159302 | UUCUUUGGCAACUUACCCAG |
| | | (Exon 2) | | 2-31593042 | |
| 28 | CR003358 | TTR | Human | chr18:3159512 | AUGCAGCUCUCCAGACUCAC |
| | | (Exon 3) | | 7-31595147 | |
| 29 | CR003359 | TTR | Human | chr18:3159512 | AGUGAGUCUGGAGAGCUGCA |
| | | (Exon 3) | | 6-31595146 | |
| 30 | CR003360 | TTR | Human | chr18:3159512 | GUGAGUCUGGAGAGCUGCAU |
| | | (Exon 3) | | 7-31595147 | |
| 31 | CR003361 | TTR | Human | chr18:3159514 | GCUGCAUGGGCUCACAACUG |
| | | (Exon 3) | | 0-31595160 | |
| 32 | CR003362 | TTR | Human | chr18:3159514 | GCAUGGGCUCACAACUGAGG |
| | | (Exon 3) | | 3-31595163 | |
| 33 | CR003363 | TTR | Human | chr18:3159515 | ACUGAGGAGGAAUUUGUAGA |
| | | (Exon 3) | | 6-31595176 | |
| 34 | CR003364 | TTR | Human | chr18:3159515 | CUGAGGAGGAAUUUGUAGAA |
| | | (Exon 3) | | 7-31595177 | |
| 35 | CR003365 | TTR | Human | chr18:3159517 | UGUAGAAGGGAUAUACAAAG |
| | | (Exon 3) | | 0-31595190 | |
| 36 | CR003366 | TTR | Human | chr18:3159519 | AAAUAGACACCAAAUCUUAC |
| | | (Exon 3) | | 3-31595213 | |
| 37 | CR003367 | TTR | Human | chr18:3159519 | AGACACCAAAUCUUACUGGA |
| | | (Exon 3) | | 7-31595217 | |
| 38 | CR003368 | TTR | Human | chr18:3159520 | AAGUGCCUUCCAGUAAGAUU |
| | | (Exon 3) | | 5-31595225 | |
| 39 | CR003369 | TTR | Human | chr18:3159523 | CUCUGCAUGCUCAUGGAAUG |
| | | (Exon 3) | | 5-31595255 | |
| 40 | CR003370 | TTR | Human | chr18:3159523 | CCUCUGCAUGCUCAUGGAAU |
| | | (Exon 3) | | 6-31595256 | |
| 41 | CR003371 | TTR | Human | chr18:3159523 | ACCUCUGCAUGCUCAUGGAA |
| | | (Exon 3) | | 7-31595257 | |
| 42 | CR003372 | TTR | Human | chr18:3159524 | UACUCACCUCUGCAUGCUCA |
| | | (Exon 3) | | 2-31595262 | |
| 43 | CR003373 | TTR | Human | chr18:3159857 | GUAUUCACAGCCAACGACUC |
| | | (Exon 4) | | 0-31598590 | |
| 44 | CR003374 | TTR | Human | chr18:3159858 | GCGGCGGGGGCCGGAGUCGU |
| | | (Exon 4) | | 3-31598603 | |
| 45 | CR003375 | TTR | Human | chr18:3159859 | AAUGGUGUAGCGGCGGGGGC |
| | | (Exon 4) | | 2-31598612 | |
| 46 | CR003376 | TTR | Human | chr18:3159859 | CGGCAAUGGUGUAGCGGCGG |
| | | (Exon 4) | | 6-31598616 | |
| 47 | CR003377 | TTR | Human | chr18:3159859 | GCGGCAAUGGUGUAGCGGCG |
| | | (Exon 4) | | 7-31598617 | |
| 48 | CR003378 | TTR | Human | chr18:3159859 | GGCGGCAAUGGUGUAGCGGC |
| | | (Exon 4) | | 8-31598618 | |
| 49 | CR003379 | TTR | Human | chr18:3159859 | GGGCGGCAAUGGUGUAGCGG |
| | | (Exon 4) | | 9-31598619 | |
| 50 | CR003380 | TTR | Human | chr18:3159860 | GCAGGGCGGCAAUGGUGUAG |
| | | (Exon 4) | | 2-31598622 | |
| 51 | CR003381 | TTR | Human | chr18:3159861 | GGGGCUCAGCAGGGCGGCAA |
| | | (Exon 4) | | 0-31598630 | |
| 52 | CR003382 | TTR | Human | chr18:3159861 | GGAGUAGGGGCUCAGCAGGG |
| | | (Exon 4) | | 6-31598636 | |
| 53 | CR003383 | TTR | Human | chr18:3159861 | AUAGGAGUAGGGGCUCAGCA |
| | | (Exon 4) | | 9-31598639 | |
| 54 | CR003384 | TTR | Human | chr18:3159862 | AAUAGGAGUAGGGGCUCAGC |
| | | (Exon 4) | | 0-31598640 | |
| 55 | CR003385 | TTR | Human | chr18:3159862 | CCCCUACUCCUAUUCCACCA |
| | | (Exon 4) | | 6-31598646 | |
| 56 | CR003386 | TTR | Human | chr18:3159862 | CCGUGGUGGAAUAGGAGUAG |
| | | (Exon 4) | | 9-31598649 | |
| 57 | CR003387 | TTR | Human | chr18:3159863 | GCCGUGGUGGAAUAGGAGUA |
| | | (Exon 4) | | 0-31598650 | |
| 58 | CR003388 | TTR | Human | chr18:3159863 | GACGACAGCCGUGGUGGAAU |
| | | (Exon 4) | | 7-31598657 | |
| 59 | CR003389 | TTR | Human | chr18:3159864 | AUUGGUGACGACAGCCGUGG |
| | | (Exon 4) | | 3-31598663 | |
| 60 | CR003390 | TTR | Human | chr18:3159864 | GGGAUUGGUGACGACAGCCG |
| | | (Exon 4) | | 6-31598666 | |
| 61 | CR003391 | TTR | Human | chr18:3159864 | GGCUGUCGUCACCAAUCCCA |
| | | (Exon 4) | | 7-31598667 | |
| 62 | CR003392 | TTR | Human | chr18:3159866 | AGUCCCUCAUUCCUUGGGAU |
| | | (Exon 4) | | 1-31598681 | |
| 63 | CR005298 | TTR | Human | chr18:3159188 | UCCACUCAUUCUUGGCAGGA |
| | | (Exon 1) | | 3-31591903 | |
| 64 | CR005299 | TTR | Human | chr18:3159863 | AGCCGUGGUGGAAUAGGAGU |
| | | (Exon 4) | | 1-31598651 | |
| 65 | CR005300 | TTR | Human | chr18:3159196 | UCACAGAAACACUCACCGUA |
| | | (Exon 1) | | 7-31591987 | |
| 66 | CR005301 | TTR | Human | chr18:3159196 | GUCACAGAAACACUCACCGU |
| | | (Exon 1) | | 8-31591988 | |
| 67 | CR005302 | TTR | Human | chr18:3159287 | ACGUGUCUUCUCUACACCCA |
| | | (Exon 2) | | 4-31592894 | |
| 68 | CR005303 | TTR | Human | chr18:3159290 | UGAAUCCAAGUGUCCUCUGA |
| | | (Exon 2) | | 3-31592923 | |
| 69 | CR005304 | TTR | Human | chr18:3159296 | GGCCGUGCAUGUGUUCAGAA |
| | | (Exon 2) | | 9-31592989 | |
| 70 | CR005305 | TTR | Human | chr18:3159511 | UAUAGGAAAACCAGUGAGUC |
| | | (Exon 3) | | 4-31595134 | |
| 71 | CR005306 | TTR | Human | chr18:3159520 | AAAUCUUACUGGAAGGCACU |
| | | (Exon 3) | | 4-31595224 | |
| 72 | CR005307 | TTR | Human | chr18:3159854 | UGUCUGUCUUCUCUCAUAGG |
| | | (Exon 4) | | 8-31598568 | |
| 73 | CR000689 | TTR | Cyno | chr18:5068153 | ACACAAAUACCAGUCCAGCG |
| | | | | 3-50681553 | |
| 74 | CR005364 | TTR | Cyno | chr18:5068048 | AAAGGCUGCUGAUGAGACCU |
| | | | | 1-50680501 | |
| 75 | CR005365 | TTR | Cyno | chr18:5068052 | CAUUGACAGCAGGACUGCCU |
| | | | | 0-50680540 | |
| 76 | CR005366 | TTR | Cyno | chr18:5068153 | AUACCAGUCCAGCGAGGCAG |
| | | | | 9-50681559 | |
| 77 | CR005367 | TTR | Cyno | chr18:5068154 | CCAGUCCAGCGAGGCAGAGG |
| | | | | 2-50681562 | |
| 78 | CR005368 | TTR | Cyno | chr18:5068154 | CCUCCUCUGCCUCGCUGGAC |
| | | | | 5-50681565 | |
| 79 | CR005369 | TTR | Cyno | chr18:5068054 | AAAGUUCUAGAUGCCGUCCG |
| | | | | 0-50680560 | |
| 80 | CR005370 | TTR | Cyno | chr18:5068059 | ACUUGUCUUCUCUAUACCCA |
| | | | | 4-50680614 | |
| 81 | CR005371 | TTR | Cyno | chr18:5067821 | AAGUGACUUCCAGUAAGAUU |
| | | | | 6-50678236 | |
| 82 | CR005372 | TTR | Cyno | chr18:5068048 | AAAAGGCUGCUGAUGAGACC |
| | | | | 2-50680502 | |

Each of the Guide Sequences above may further comprise additional nucleotides to form a crRNA, e.g., with the following exemplary nucleotide sequence following the Guide Sequence at its 3′ end: GUUUUAGAGCUAUGCUGUUUUG (SEQ ID NO: 126). In the case of a sgRNA, the above Guide Sequences may further comprise additional nucleotides to form a sgRNA, e.g., with the following exemplary nucleotide sequence following the 3′ end of the Guide Sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 125) in 5′ to 3′ orientation.

In some embodiments, the sgRNA is modified. In some embodiments, the sgRNA comprises the modification pattern shown below in SEQ ID NO: 3, where N is any natural or non-natural nucleotide, and where the totality of the N's comprise a guide sequence as described herein and the modified sgRNA comprises the following sequence: mN*mN*mN*NNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 3), where “N” may be any natural or non-natural nucleotide. For example, encompassed herein is SEQ ID NO: 3, where the N's are replaced with any of the guide sequences disclosed herein. The modifications remain as shown in SEQ ID NO: 3 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, any one of the sequences recited in Table 2 is encompassed.

TABLE 2
TTR targeted sgRNA sequences

| SEQ | | | | |
|---|---|---|---|---|
| ID | | Target and | | |
| No. | Guide ID | Description | Species | Sequence |
| 87 | G000480 | TTR | Human | mA*mA*mA*GGCUGCUGAUGACACCUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 88 | G000481 | TTR | Human | mU*mC*mU*AGAACUUUGACCAUCAGGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 89 | G000482 | TTR | Human | mU*mG*mU*AGAAGGGAUAUACAAAGG |
| | | sgRNA | | UUUUAGAmGmCmUmAmGmAmAmAmUm |
| | | modified | | AmGmCAAGUUAAAAUAAGGCUAGUCCG |
| | | sequence | | UUAUCAmAmCmUmUmGmAmAmAmAmA |
| | | | | mGmUmGmGmCmAmCmCmGmAmGmUmC |
| | | | | mGmGmUmGmCmU*mU*mU*mU |
| 90 | G000483 | TTR | Human | mU*mC*mC*ACUCAUUCUUGGCAGGAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 91 | G000484 | TTR | Human | mA*mG*mA*CACCAAAUCUUACUGGAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 92 | G000485 | TTR | Human | mC*mC*mU*CCUCUGCCUUGCUGGACGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 93 | G000486 | TTR | Human | mA*mC*mA*CAAAUACCAGUCCAGCAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 94 | G000487 | TTR | Human | mU*mU*mC*UUUGGCAACUUACCCAGGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 95 | G000488 | TTR | Human | mA*mA*mA*GUUCUAGAUGCUGUCCGGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 96 | G000489 | TTR | Human | mU*mU*mU*GACCAUCAGAGGACACUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 97 | G000490 | TTR | Human | mA*mA*mA*UAGACACCAAAUCUUACGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 98 | G000491 | TTR | Human | mA*mU*mA*CCAGUCCAGCAAGGCAGGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 99 | G000492 | TTR | Human | mC*mU*mU*CUCUACACCCAGGGCACGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 100 | G000493 | TTR | Human | mA*mA*mG*UGCCUUCCAGUAAGAUUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 101 | G000494 | TTR | Human | mG*mU*mG*AGUCUGGAGAGCUGCAUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 102 | G000495 | TTR | Human | mC*mA*mG*AGGACACUUGGAUUCACGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 103 | G000496 | TTR | Human | mG*mG*mC*CGUGCAUGUGUUCAGAAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 104 | G000497 | TTR | Human | mC*mU*mG*CUCCUCCUCUGCCUUGCGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 105 | G000498 | TTR | Human | mA*mG*mU*GAGUCUGGAGAGCUGCAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 106 | G000499 | TTR | Human | mU*mG*mA*AUCCAAGUGUCCUCUGAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 107 | G000500 | TTR | Human | mC*mC*mA*GUCCAGCAAGGCAGAGGGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 108 | G000501 | TTR | Human | mU*mC*mA*CAGAAACACUCACCGUAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 109 | G000567 | TTR | Human | mG*mA*mA*AGGCUGCUGAUGACACCGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 110 | G000568 | TTR | Human | mG*mG*mC*UGUCGUCACCAAUCCCAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 111 | G000570 | TTR | Human | mC*mA*mU*UGAUGGCAGGACUGCCUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 112 | G000571 | TTR | Human | mG*mU*mC*ACAGAAACACUCACCGUGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 113 | G000572 | TTR | Human | mC*mC*mC*CUACUCCUAUUCCACCAGU |
| | | sgRNA | | UUUAGAmGmCmUmAmGmAmAmAmUmA |
| | | modified | | mGmCAAGUUAAAAUAAGGCUAGUCCGU |
| | | sequence | | UAUCAmAmCmUmUmGmAmAmAmAmAm |
| | | | | GmUmGmGmCmAmCmCmGmAmGmUmCm |
| | | | | GmGmUmGmCmU*mU*mU*mU |
| 114 | G000502 | TTR Cyno | Cyno | mA*mC*mA*CAAAUACCAGUCCAGCGGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 115 | G000503 | TTR Cyno | Cyno | mA*mA*mA*AGGCUGCUGAUGAGACCGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 116 | G000504 | TTR Cyno | Cyno | mA*mA*mA*GGCUGCUGAUGAGACCUGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 117 | G000505 | TTR Cyno | Cyno | mC*mA*mU*UGACAGCAGGACUGCCUGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 118 | G000506 | TTR Cyno | Cyno | mA*mU*mA*CCAGUCCAGCGAGGCAGGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 119 | G000507 | TTR Cyno | Cyno | mC*mC*mA*GUCCAGCGAGGCAGAGGGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 120 | G000508 | TTR Cyno | Cyno | mC*mC*mU*CCUCUGCCUCGCUGGACGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 121 | G000509 | TTR Cyno | Cyno | mA*mA*mA*GUUCUAGAUGCCGUCCGGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 122 | G000510 | TTR Cyno | Cyno | mA*mC*mU*UGUCUUCUCUAUACCCAGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 123 | G000511 | TTR Cyno | Cyno | mA*mA*mG*UGACUUCCAGUAAGAUUGU |
| | | specific | | UUUAGAmGmCmUmAmGmAmAmAmUmA |
| | | sgRNA | | mGmCAAGUUAAAAUAAGGCUAGUCCGU |
| | | modified | | UAUCAmAmCmUmUmGmAmAmAmAmAm |
| | | sequence | | GmUmGmGmCmAmCmCmGmAmGmUmCm |
| | | | | GmGmUmGmCmU*mU*mU*mU |
| 124 | G000282 | TTR | Mouse | mU*mU*mA*CAGCCACGUCUACAGCAGU |
| | | | | UUUAGAmGmCmUmAmGmAmAmAmUmA |
| | | | | mGmCAAGUUAAAAUAAGGCUAGUCCGU |
| | | | | UAUCAmAmCmUmUmGmAmAmAmAmAm |
| | | | | GmUmGmGmCmAmCmCmGmAmGmUmCm |
| | | | | GmGmUmGmCmU*mU*mU*mU |

* = PS linkage;
‘m’ = 2′-O-Me nucleotide

An alignment mapping of the Guide IDs with the corresponding sgRNA IDs as well as homology to the cyno genome and cyno matched guide IDs are provided in Table 3.

TABLE 3
TTR targeted guide sequence ID mapping and Cyno Homology

| | Human | Human | Number | Cyno | Cyno |
|---|---|---|---|---|---|
| | Dual | Single | Mismatches | Matched | Matched |
| Descrip- | Guide | Guide | to Cyno | dgRNA | sgRNA |
| tion | ID | ID | Genome | ID | ID |
| TTR | CR003335 | G000497 | 1 | | |
| TTR | CR003336 | G000485 | 1 | CR005368 | G000508 |
| TTR | CR003337 | G000500 | 1 | CR005367 | G000507 |
| TTR | CR003338 | G000491 | 1 | CR005366 | G000506 |
| TTR | CR003339 | G000486 | 1 | CR000689 | G000502 |
| TTR | CR003340 | | 0 | | |
| TTR | CR003341 | | 0 | | |
| TTR | CR003342 | G000492 | no PAM | | |
| | | | in cyno | | |
| TTR | CR003343 | G000495 | no PAM | | |
| | | | in cyno | | |
| TTR | CR003344 | G000489 | 0 | | |
| TTR | CR003345 | G000481 | 0 | | |
| TTR | CR003346 | G000488 | 1 | CR005369 | G000509 |
| TTR | CR003347 | G000570 | 2 | CR005365 | G000505 |
| TTR | CR003348 | | 2 | | |
| TTR | CR003349 | | >3 | | |
| TTR | CR003350 | | no PAM | | |
| | | | in cyno | | |
| TTR | CR003351 | | no PAM | | |
| | | | in cyno | | |
| TTR | CR003352 | G000567 | 2 | CR005372 | G000503 |
| TTR | CR003353 | G000480 | 1 | CR005364 | G000504 |
| TTR | CR003354 | | 1 | | |
| TTR | CR003355 | | 1 | | |
| TTR | CR003356 | | 3 | | |
| TTR | CR003357 | G000487 | >3 | | |
| TTR | CR003358 | | 0 | | |
| TTR | CR003359 | G000498 | 0 | | |
| TTR | CR003360 | G000494 | 0 | | |
| TTR | CR003361 | | 0 | | |
| TTR | CR003362 | | 0 | | |
| TTR | CR003363 | | 0 | | |
| TTR | CR003364 | | 0 | | |
| TTR | CR003365 | G000482 | 0 | | |
| TTR | CR003366 | G000490 | 0 | | |
| TTR | CR003367 | G000484 | no PAM | | |
| | | | in cyno | | |
| TTR | CR003368 | G000493 | 1 | CR005371 | G000511 |
| TTR | CR003369 | | 0 | | |
| TTR | CR003370 | | 0 | | |
| TTR | CR003371 | | 0 | | |
| TTR | CR003372 | | 0 | | |
| TTR | CR003373 | | 1 | | |
| TTR | CR003374 | | 2 | | |
| TTR | CR003375 | | 2 | | |
| TTR | CR003376 | | 2 | | |
| TTR | CR003377 | | 2 | | |
| TTR | CR003378 | | 2 | | |
| TTR | CR003379 | | 2 | | |
| TTR | CR003380 | | 1 | | |
| TTR | CR003381 | | 1 | | |
| TTR | CR003382 | | 0 | | |
| TTR | CR003383 | | 0 | | |
| TTR | CR003384 | | 0 | | |
| TTR | CR003385 | G000572 | 0 | | |
| TTR | CR003386 | | 0 | | |
| TTR | CR003387 | | 0 | | |
| TTR | CR003388 | | 0 | | |
| TTR | CR003389 | G000569 | 0 | | |
| TTR | CR003390 | | 0 | | |
| TTR | CR003391 | G000568 | 0 | | |
| TTR | CR003392 | | 0 | | |
| TTR | CR005298 | G000483 | 1 | | |
| TTR | CR005299 | | 0 | | |
| TTR | CR005300 | G000501 | no PAM | | |
| | | | in cyno | | |
| TTR | CR005301 | G000571 | 0 | | |
| TTR | CR005302 | | 2 | CR005370 | G000510 |
| TTR | CR005303 | G000499 | 0 | | |
| TTR | CR005304 | G000496 | >3 | | |
| TTR | CR005305 | | 0 | | |
| TTR | CR005306 | | 1 | | |
| TTR | CR005307 | | 0 | | |

In some embodiments, the gRNA comprises a guide sequence that direct an RNA-guided DNA binding agent, which can be a nuclease (e.g., a Cas nuclease such as Cas9), to a target DNA sequence in TTR. The gRNA may comprise a crRNA comprising a guide sequence shown in Table 1. The gRNA may comprise a crRNA comprising 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to a guide sequence shown in Table 1. The gRNA may further comprise a trRNA. In each composition and method embodiment described herein, the crRNA and trRNA may be associated as a single RNA (sgRNA), or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1, and a second RNA molecule comprising a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1 covalently linked to a trRNA. The sgRNA may comprise 17, 18, 19, or 20 contiguous nucleotides of a guide sequence shown in Table 1. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the composition comprises one or more guide RNAs comprising a guide sequence selected from SEQ ID NOs: 5-82.

In some embodiments, the composition comprises a gRNA that comprises a guide sequence that is at least 99%, 98%, 97%, 96%, 95%, 94%, 93%, 92%, 91%, or 90% identical to a sequence selected from SEQ ID NOs: 5-82.

In some embodiments, the composition comprises one or more guide RNAs comprising a guide sequence selected from SEQ ID NOs: 5-72, 74-78, and 80-82. In some embodiments, the composition comprises a gRNA that comprises a guide sequence that is at least 99%, 98%, 97%, 96%, 95%, 94%, 93%, 92%, 91%, or 90% identical to a sequence selected from SEQ ID NOs: 5-72, 74-78, and 80-82. In some embodiments, the sequence selected from SEQ ID NOs: 5-72, 74-78, and 80-82 is SEQ ID NOs: 5, 6, 7, 8, 9, 12, 13, 14, 15, 16, 17, 22, 23, 27, 29, 30, 35, 36, 37, 38, 55, 61, 63, 65, 66, 68, or 69. In some embodiments, the sequence selected from SEQ ID NOs: 5-72, 74-78, and 80-82 is SEQ ID NO: 5, 6, 9, 13, 14, 15, 16, 17, 22, 23, 27, 30, 35, 36, 37, 38, 55, 63, 65, 66, 68, or 69.

In other embodiments, the composition comprises at least one, e.g., at least two gRNAs comprising guide sequences selected from any two or more of the guide sequences of SEQ ID NOs: 5-82. In some embodiments, the composition comprises at least two gRNAs that each comprise a guide sequence at least 99%, 98%, 97%, 96%, 95%, 94%, 93%, 92%, 91%, or 90% identical to a sequence selected from SEQ ID NOs: 5-82.

In other embodiments, the composition comprises at least one, e.g., at least two gRNAs comprising guide sequences selected from any two or more of the guide sequences selected from SEQ ID NOs: 5-72, 74-78, and 80-82. In some embodiments, the composition comprises at least two gRNAs that each comprise a guide sequence at least 99%, 98%, 97%, 96%, 95%, 94%, 93%, 92%, 91%, or 90% identical to any of the sequences selected from SEQ ID NOs: 5-72, 74-78, and 80-82. In some embodiments, the sequences selected from SEQ ID NOs: 5-72, 74-78, and 80-82 comprise a sequence, or two sequences, selected from SEQ ID NOs: 5, 6, 7, 8, 9, 12, 13, 14, 15, 16, 17, 22, 23, 27, 29, 30, 35, 36, 37, 38, 55, 61, 63, 65, 66, 68, or 69. In some embodiments, the sequence selected from SEQ ID NOs: 5-72, 74-78, and 80-82 comprise a sequence, or two sequences, selected from SEQ ID NO: 5, 6, 9, 13, 14, 15, 16, 17, 22, 23, 27, 30, 35, 36, 37, 38, 55, 63, 65, 66, 68, or 69.

In some embodiments, the gRNA is a sgRNA comprising any one of the sequences shown in Table 2 (SEQ ID Nos. 87-124). In some embodiments, the gRNA is a sgRNA comprising any one of the sequences shown in Table 2 (SEQ ID Nos. 87-124, but without the modifications as shown (i.e., unmodified SEQ ID Nos. 87-124). In some embodiments, the sgRNA comprises a sequence that is at least 99%, 98%, 97%, 96%, 95%, 94%, 93%, 92%, 91%, or 90% identical to any of the nucleic acids of SEQ ID Nos. 87-124. In some embodiments, the sgRNA comprises a sequence that is at least 99%, 98%, 97%, 96%, 95%, 94%, 93%, 92%, 91%, or 90% identical to any of the nucleic acids of SEQ ID Nos. 87-124, but without the modifications as shown (i.e., unmodified SEQ ID Nos. 87-124). In some embodiments, the sgRNA comprises any one of the guide sequences shown in Table 1 in place of the guide sequences shown in the sgRNA sequences of Table 2 at SEQ ID Nos: 87-124, with or without the modifications.

In some embodiments, the gRNA is a sgRNA comprising any one of SEQ ID Nos. 87-113, 115-120, or 122-124. In some embodiments, the gRNA is a sgRNA comprising any one of SEQ ID Nos. 87-113, 115-120, or 122-124, but without the modifications as shown in Table 2 (i.e., unmodified SEQ ID Nos. 87-113, 115-120, or 122-124). In some embodiments, the sgRNA comprises a sequence that is at least 99%, 98%, 97%, 96%, 95%, 94%, 93%, 92%, 91%, or 90% identical to any of the nucleic acids of SEQ ID Nos. 87-113, 115-120, or 122-124. In some embodiments, the sgRNA comprises a sequence that is at least 99%, 98%, 97%, 96%, 95%, 94%, 93%, 92%, 91%, or 90% identical to any of the nucleic acids of SEQ ID Nos. 87-113, 115-120, or 122-124, but without the modifications as shown (i.e., unmodified SEQ ID Nos. 87-113, 115-120, or 122-124). In some embodiments, the sgRNA comprises any one of the guide sequences shown in Table 1 in place of the guide sequences shown in the sgRNA sequences of Table 2 at SEQ ID Nos: 87-113, 115-120, or 122-124, with or without the modifications.

The guide RNAs provided herein can be useful for recognizing (e.g., hybridizing to) a target sequence in the TTR gene. For example, the TTR target sequence may be recognized and cleaved by a provided Cas cleavase comprising a guide RNA. Thus, an RNA-guided DNA binding agent, such as a Cas cleavase, may be directed by a guide RNA to a target sequence of the TTR gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the TTR gene.

Without being bound by any particular theory, mutations (e.g., frameshift mutations resulting from indels occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within TTR is used to direct the RNA-guided DNA binding agent to a particular location in the TTR gene. In some embodiments, gRNAs are designed to have guide sequences that are complementary or have complementarity to target sequences in exon 1, exon 2, exon 3, or exon 4 of TTR.

In some embodiments, the guide sequence is at least 99%, 98%, 97%, 96%, 95%, 94%, 93%, 92%, 91%, or 90% identical to a target sequence present in the human TTR gene. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

C. Modifications of gRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the nonbridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, acylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides.

In some embodiments, the guide RNAs disclosed herein comprise one of the modification patterns disclosed in U.S. 62/431,756, filed Dec. 8, 2016, titled “Chemically Modified Guide RNAs,” the contents of which are hereby incorporated by reference in their entirety.

In some embodiments, the invention comprises a gRNA comprising one or more modifications. In some embodiments, the modification comprises a 2′-O-methyl (2′-O-Me) modified nucleotide. In some embodiments, the modification comprises a phosphorothioate (PS) bond between nucleotides.

The terms “mA,” “mC,” “mU,” or “mG” may be used to denote a nucleotide that has been modified with 2′-O-Me.

Modification of 2′-O-methyl can be depicted as follows:

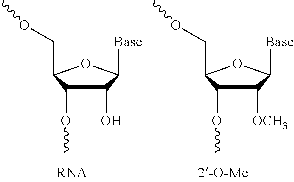

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

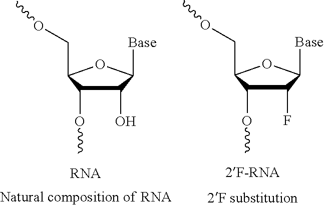

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a nonbridging phosphate oxygen, generating a PS bond in lieu of a phosphodiester bond:

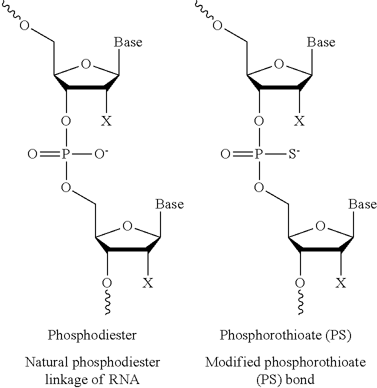

Abasic nucleotides refer to those which lack nitrogenous bases. The figure below depicts an oligonucleotide with an abasic (also known as apurinic) site that lacks a base:

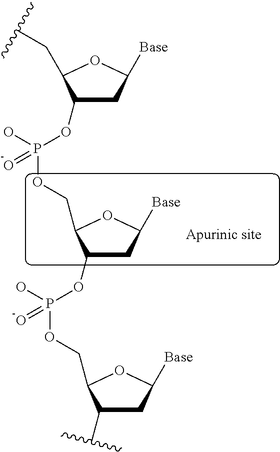

Inverted bases refer to those with linkages that are inverted from the normal 5′ to 3′ linkage (i.e., either a 5′ to 5′ linkage or a 3′ to 3′ linkage). For example:

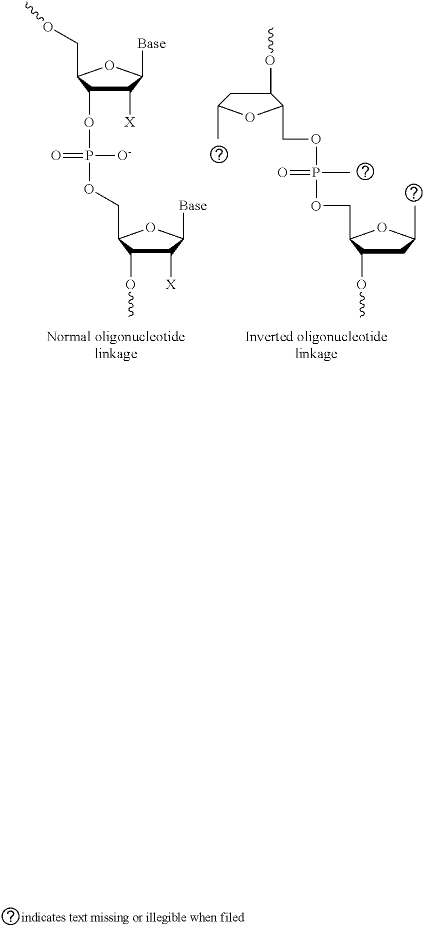

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID No: 3, where N is any natural or non-natural nucleotide, and where the totality of the N's comprise a guide sequence that directs a nuclease to a target sequence.

In some embodiments, the guide RNA comprises a sgRNA shown in any one of SEQ ID No: 87-124. In some embodiments, the guide RNA comprises a sgRNA comprising any one of the guide sequences of SEQ ID No: 5-82 and the nucleotides of SEQ ID No: 125, wherein the nucleotides of SEQ ID No: 125 are on the 3′ end of the guide sequence, and wherein the guide sequence may be modified as shown in SEQ ID No: 3.

In some embodiments, the guide RNA comprises a sgRNA comprising a guide sequence selected from SEQ ID Nos: 5-72, 74-78, and 80-82 and nucleotides 21-100 of SEQ ID No: 3, wherein the nucleotides of SEQ ID No: 3 are on the 3′ end of the guide sequence, and wherein the guide sequence may be modified as shown in SEQ ID No: 3.

D. RNA-Guided DNA Binding Agent

In some embodiments, the RNA-guided DNA-binding agent is a Class 2 Cas nuclease. In some embodiments, the RNA-guided DNA-binding agent has cleavase activity, which can also be referred to as double-strand endonuclease activity. In some embodiments, the RNA-guided DNA-binding agent comprises a Cas nuclease, such as a Class 2 Cas nuclease (which may be, e.g., a Cas nuclease of Type II, V, or VI). Class 2 Cas nucleases include, for example, Cas9, Cpf1, C2c1, C2c2, and C2c3 proteins and modifications thereof. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., Nat. Rev. Microbiol. 9:467-477 (2011); Makarova et al., Nat. Rev. Microbiol, 13: 722-36 (2015); Shmakov et al., Molecular Cell, 60:385-397 (2015). In some embodiments, the RNA-guided DNA binding agent is a Cas cleavase, e.g. a Cas9 cleavase. In some embodiments, the RNA-guided DNA binding agent is a Cas nickase, e.g. a Cas9 nickase. In some embodiments, the RNA-guided DNA binding agent is a Cas9 nuclease, such as a cleavase or nickase. In some embodiments, the RNA-guided DNA binding agent is an S. pyogenes Cas9 nuclease, e.g. a cleavase.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In certain embodiments, the Cas nuclease may cleave dsDNA, it may cleave one strand of dsDNA, or it may not have DNA cleavase or nickase activity. An exemplary Cas9 amino acid sequence is provided as SEQ ID NO: 203. An exemplary Cas9 mRNA ORF sequence, which includes start and stop codons, is provided as SEQ ID NO: 311. An exemplary Cas9 mRNA coding sequence, suitable for inclusion in a fusion protein, is provided as SEQ ID NO: 210.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain. An exemplary Cas9 nickase amino acid sequence is provided as SEQ ID NO: 206. An exemplary Cas9 nickase mRNA ORF sequence, which includes start and stop codons, is provided as SEQ ID NO: 207. An exemplary Cas9 nickase mRNA coding sequence, suitable for inclusion in a fusion protein, is provided as SEQ ID NO: 211.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)).

In some embodiments, a nucleic acid encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1. An exemplary dCas9 amino acid sequence is provided as SEQ ID NO: 208. An exemplary dCas9 mRNA ORF sequence, which includes start and stop codons, is provided as SEQ ID NO: 209. An exemplary dCas9 mRNA coding sequence, suitable for inclusion in a fusion protein, is provided as SEQ ID NO: 346.

a) Heterologous Functional Domains; Nuclear Localization Signals

In some embodiments, the RNA-guided DNA-binding agent, e.g. a Cas9 nuclease such as an S. pyogenes Cas9, comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the RNA-guided DNA-binding agent may be fused C-terminally to at least one NLS. An NLS may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 278) or PKKKRRV (SEQ ID NO: 290). In some embodiments, the NLS may be a bipartite sequence, such as the NLS of nucleoplasmin, KRPAATKKAGQAKKKK (SEQ ID NO: 91). In some embodiments, the NLS sequence may comprise LAAKRSRTT (SEQ ID NO: 279), QAAKRSRTT (SEQ ID NO: 280), PAPAKRERTT (SEQ ID NO: 281), QAAKRPRTT (SEQ ID NO: 282), RAAKRPRTT (SEQ ID NO: 283), AAAKRSWSMAA (SEQ ID NO: 284), AAAKRVWSMAF (SEQ ID NO: 285), AAAKRSWSMAF (SEQ ID NO: 286), AAAKRKYFAA (SEQ ID NO: 287), RAAKRKAFAA (SEQ ID NO: 288), or RAAKRKYFAV (SEQ ID NO: 289). In a specific embodiment, a single PKKKRKV (SEQ ID NO: 278) NLS may be linked at the C-terminus of the RNA-guided DNA-binding agent. One or more linkers are optionally included at the fusion site. In some embodiments, one or more NLS(s) according to any of the foregoing embodiments are present in the RNA-guided DNA-binding agent in combination with one or more additional heterologous functional domains, such as any of the heterologous functional domains described below.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, 51, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In certain embodiments, the DNA modification domain is a methylation domain, such as a demethylation or methyltransferase domain. In certain embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and US 2016/0304846 are hereby incorporated by reference.

E. Nucleic Acid Comprising an Open Reading Frame Encoding an RNA-Guided DNA Binding Agent

Any nucleic acid comprising an ORF encoding an RNA-guided DNA binding agent disclosed herein, e.g. a Cas9 nuclease such as an S. pyogenes Cas9, may be optionally combined in a composition or method with any of the gRNAs disclosed herein. In any of the embodiments set forth herein, the nucleic acid comprising an open reading frame encoding an RNA-guided DNA binding agent may be an mRNA.

1. ORFs with Low Adenine Content

In some embodiments, the ORF encoding the RNA-guided DNA-binding agent, e.g. a Cas9 nuclease such as an S. pyogenes Cas9, has an adenine content ranging from its minimum adenine content to about 150% of its minimum adenine content. In some embodiments, the adenine content of the ORF is less than or equal to about 145%, 140%, 135%, 130%, 125%, 120%, 115%, 110%, 105%, 104%, 103%, 102%, or 101% of its minimum adenine content. In some embodiments, the ORF has an adenine content equal to its minimum adenine content. In some embodiments, the ORF has an adenine content less than or equal to about 150% of its minimum adenine content. In some embodiments, the ORF has an adenine content less than or equal to about 145% of its minimum adenine content. In some embodiments, the ORF has an adenine content less than or equal to about 140% of its minimum adenine content. In some embodiments, the ORF has an adenine content less than or equal to about 135% of its minimum adenine content. In some embodiments, the ORF has an adenine content less than or equal to about 130% of its minimum adenine content. In some embodiments, the ORF has an adenine content less than or equal to about 125% of its minimum adenine content. In some embodiments, the ORF has an adenine content less than or equal to about 120% of its minimum adenine content. In some embodiments, the ORF has an adenine content less than or equal to about 115% of its minimum adenine content. In some embodiments, the ORF has an adenine content less than or equal to about 110% of its minimum adenine content. In some embodiments, the ORF has an adenine content less than or equal to about 105% of its minimum adenine content. In some embodiments, the ORF has an adenine content less than or equal to about 104% of its minimum adenine content. In some embodiments, the ORF has an adenine content less than or equal to about 103% of its minimum adenine content. In some embodiments, the ORF has an adenine content less than or equal to about 102% of its minimum adenine content. In some embodiments, the ORF has an adenine content less than or equal to about 101% of its minimum adenine content.

In some embodiments, the ORF has an adenine dinucleotide content ranging from its minimum adenine dinucleotide content to 200% of its minimum adenine dinucleotide content. In some embodiments, the adenine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum adenine dinucleotide content. In some embodiments, the ORF has an adenine dinucleotide content equal to its minimum adenine dinucleotide content. In some embodiments, the ORF has an adenine dinucleotide content less than or equal to about 200% of its minimum adenine dinucleotide content. In some embodiments, the ORF has an adenine dinucleotide content less than or equal to about 195% of its minimum adenine dinucleotide content. In some embodiments, the ORF has an adenine dinucleotide content less than or equal to about 190% of its minimum adenine dinucleotide content. In some embodiments, the ORF has an adenine dinucleotide content less than or equal to about 185% of its minimum adenine dinucleotide content. In some embodiments, the ORF has an adenine dinucleotide content less than or equal to about 180% of its minimum adenine dinucleotide content. In some embodiments, the ORF has an adenine dinucleotide content less than or equal to about 175% of its minimum adenine dinucleotide content. In some embodiments, the ORF has an adenine dinucleotide content less than or equal to about 170% of its minimum adenine dinucleotide content. In some embodiments, the ORF has an adenine dinucleotide content less than or equal to about 165% of its minimum adenine dinucleotide content. In some embodiments, the ORF has an adenine dinucleotide content less than or equal to about 160% of its minimum adenine dinucleotide content. In some embodiments, the ORF has an adenine dinucleotide content less than or equal to about 155% of its minimum adenine dinucleotide content. In some embodiments, the ORF has an adenine dinucleotide content equal to its minimum adenine dinucleotide content. In some embodiments, the ORF has an adenine dinucleotide content less than or equal to about 150% of its minimum adenine dinucleotide content. In some embodiments, the ORF has an adenine dinucleotide content less than or equal to about 145% of its minimum adenine dinucleotide content. In some embodiments, the ORF has an adenine dinucleotide content less than or equal to about 140% of its minimum adenine dinucleotide content. In some embodiments, the ORF has an adenine dinucleotide content less than or equal to about 135% of its minimum adenine dinucleotide content. In some embodiments, the ORF has an adenine dinucleotide content less than or equal to about 130% of its minimum adenine dinucleotide content. In some embodiments, the ORF has an adenine dinucleotide content less than or equal to about 125% of its minimum adenine dinucleotide content. In some embodiments, the ORF has an adenine dinucleotide content less than or equal to about 120% of its minimum adenine dinucleotide content. In some embodiments, the ORF has an adenine dinucleotide content less than or equal to about 115% of its minimum adenine dinucleotide content. In some embodiments, the ORF has an adenine dinucleotide content less than or equal to about 110% of its minimum adenine dinucleotide content. In some embodiments, the ORF has an adenine dinucleotide content less than or equal to about 105% of its minimum adenine dinucleotide content. In some embodiments, the ORF has an adenine dinucleotide content less than or equal to about 104% of its minimum adenine dinucleotide content. In some embodiments, the ORF has an adenine dinucleotide content less than or equal to about 103% of its minimum adenine dinucleotide content. In some embodiments, the ORF has an adenine dinucleotide content less than or equal to about 102% of its minimum adenine dinucleotide content. In some embodiments, the ORF has an adenine dinucleotide content less than or equal to about 101% of its minimum adenine dinucleotide content.

In some embodiments, the ORF has an adenine dinucleotide content ranging from its minimum adenine dinucleotide content to the adenine dinucleotide content that is 90% or lower of the maximum adenine dinucleotide content of a reference sequence that encodes the same protein as the mRNA in question. In some embodiments, the adenine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine dinucleotide content of a reference sequence that encodes the same protein as the mRNA in question.

In some embodiments, the ORF has an adenine trinucleotide content ranging from 0 adenine trinucleotides to 1, 2, 3, 4, 5, 6, 7, 8, 9, 10, 20, 30, 40, or 50 adenine trinucleotides (where a longer run of adenines counts as the number of unique three-adenine segments within it, e.g., an adenine tetranucleotide contains two adenine trinucleotides, an adenine pentanucleotide contains three adenine trinucleotides, etc.). In some embodiments, the ORF has an adenine trinucleotide content ranging from 0% adenine trinucleotides to 0.1%, 0.2%, 0.3%, 0.4%, 0.5%, 0.6%, 0.7%, 0.8%, 0.9%, 1%, 1.5%, or 2% adenine trinucleotides, where the percentage content of adenine trinucleotides is calculated as the percentage of positions in a sequence that are occupied by adenines that form part of an adenine trinucleotide (or longer run of adenines), such that the sequences UUUAAA and UUUUAAAA would each have an adenine trinucleotide content of 50%. For example, in some embodiments, the ORF has an adenine trinucleotide content less than or equal to 2%. For example, in some embodiments, the ORF has an adenine trinucleotide content less than or equal to 1.5%. In some embodiments, the ORF has an adenine trinucleotide content less than or equal to 1%. In some embodiments, the ORF has an adenine trinucleotide content less than or equal to 0.9%. In some embodiments, the ORF has an adenine trinucleotide content less than or equal to 0.8%. In some embodiments, the ORF has an adenine trinucleotide content less than or equal to 0.7%. In some embodiments, the ORF has an adenine trinucleotide content less than or equal to 0.6%. In some embodiments, the ORF has an adenine trinucleotide content less than or equal to 0.5%. In some embodiments, the ORF has an adenine trinucleotide content less than or equal to 0.4%. In some embodiments, the ORF has an adenine trinucleotide content less than or equal to 0.3%. In some embodiments, the ORF has an adenine trinucleotide content less than or equal to 0.2%. In some embodiments, the ORF has an adenine trinucleotide content less than or equal to 0.1%. In some embodiments, a nucleic acid is provided that encodes an RNA-guided DNA-binding agent comprising an ORF containing no adenine trinucleotides.

In some embodiments, the ORF has an adenine trinucleotide content ranging from its minimum adenine trinucleotide content to the adenine trinucleotide content that is 90% or lower of the maximum adenine trinucleotide content of a reference sequence that encodes the same protein as the mRNA in question. In some embodiments, the adenine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine trinucleotide content of a reference sequence that encodes the same protein as the mRNA in question.

A given ORF can be reduced in adenine content or adenine dinucleotide content or adenine trinucleotide content, for example, by using minimal adenine codons in a sufficient fraction of the ORF. For example, an amino acid sequence for an RNA-guided DNA-binding agent can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 4.

TABLE 4
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, a nucleic acid is provided that encodes an RNA-guided DNA-binding agent, e.g. a Cas9 nuclease such as an S. pyogenes Cas9, comprising an ORF consisting of a set of codons of which at least about 75%, 80%, 85%, 90%, 95%, 98%, 99%, or 100% of the codons are codons listed in Table 4. In some embodiments, the ORF has minimal nucleotide homopolymers, e.g., repetitive strings of the same nucleotides. For example, in some embodiments, when selecting a minimal uridine codon from the codons listed in Table 4, a nucleic acid is constructed by selecting the minimal adenine codons that reduce the number and length of nucleotide homopolymers, e.g., selecting GCG instead of GCC for alanine or selecting GGC instead of GGG for glycine.

In any of the foregoing embodiments, the nucleic acid may be an mRNA.

2. Codons that Increase Translation and/or that Correspond to Highly Expressed tRNAs; Exemplary Codon Sets

In some embodiments, the nucleic acid comprises an ORF having codons that increase translation in a mammal, such as a human. In further embodiments, the nucleic acid comprises an ORF having codons that increase translation in an organ, such as the liver, of the mammal, e.g., a human. In further embodiments, the nucleic acid comprises an ORF having codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level, such as S. pyogenes, S. aureus, or another prokaryote as the case may be for prokaryotically-derived Cas nucleases, such as the Cas nucleases from other prokaryotes described below. Alternatively, in some embodiments, an increase in translation for a Cas9 sequence in a mammal, cell type, organ of a mammal, human, organ of a human, etc., is determined relative to translation of an ORF with the sequence of SEQ ID NO: 205 with all else equal, including any applicable point mutations, heterologous domains, and the like. Codons useful for increasing expression in a human, including the human liver and human hepatocytes, can be codons corresponding to highly expressed tRNAs in the human liver/hepatocytes, which are discussed in Dittmar K A, PLos Genetics 2(12): e221 (2006). In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian liver, such as a human liver. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian hepatocyte, such as a human hepatocyte.

Alternatively, codons corresponding to highly expressed tRNAs in an organism (e.g., human) in general may be used.

Any of the foregoing approaches to codon selection can be combined with the minimal adenine codons shown above, e.g., by starting with the codons of Table 4, and then where more than one option is available, using the codon that corresponds to a more highly-expressed tRNA, either in the organism (e.g., human) in general, or in an organ or cell type of interest, such as the liver or hepatocytes (e.g., human liver or human hepatocytes).

In some embodiments, at least 75%, 80%, 85%, 90%, 95%, 96%, 97%, 98%, 99%, or 100% of the codons in an ORF are codons from a codon set shown in Table 5 (e.g., the low U 1, low A, or low A/U codon set). The codons in the low U 1, low G, low C, low A, and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U 1 codon set shown in Table 5. In some embodiments, at least 75%, 80%, 85%, 90%, 95%, 96%, 97%, 98%, 99%, or 100% of the codons in an ORF are codons from the low A codon set shown in Table 5. In some embodiments, at least 75%, 80%, 85%, 90%, 95%, 96%, 97%, 98%, 99%, or 100% of the codons in an ORF are codons from the low A/U codon set shown in Table 5.

TABLE 5
Exemplary Codon Sets

| Amino | Low | Low | High | Low | Low | Low | Low | Long |
|---|---|---|---|---|---|---|---|---|
| Acid | U 1 | U 2 | U | G | C | A | A/U | Half Life |
| Gly | GGC | GGG | GGT | GGC | GGA | GGC | GGC | GGT |
| Glu | GAG | GAA | GAA | GAA | GAG | GAG | GAG | GAA |
| Asp | GAC | GAC | GAT | GAC | GAT | GAC | GAC | GAC |
| Val | GTG | GTA | GTT | GTC | GTG | GTG | GTG | GTC |
| Ala | GCC | GCG | GCT | GCC | GCT | GCC | GCC | GCC |
| Arg | AGA | CGA | CGT | AGA | AGA | CGG | CGG | AGA |
| Ser | AGC | AGC | TCT | TCC | AGT | TCC | AGC | TCT |
| Lys | AAG | AAA | AAA | AAA | AAG | AAG | AAG | AAG |
| Asn | AAC | AAC | AAT | AAC | AAT | AAC | AAC | AAC |
| Met | ATG | ATG | ATG | ATG | AGT | ATG | ATG | ATG |
| Ile | ATC | ATA | ATT | ATC | ATT | ATC | ATC | ATC |
| Thr | ACC | ACG | ACT | ACC | ACA | ACC | ACC | ACC |
| Trp | TGG | TGG | TGG | TGG | TGG | TGG | TGG | TGG |
| Cys | TGC | TGC | TGT | TGC | TGT | TGC | TGC | TGC |
| Tyr | TAC | TAC | TAT | TAC | TAT | TAC | TAC | TAC |
| Leu | CTG | CTA | TTA | CTC | TTG | CTG | CTG | TTG |
| Phe | TTC | TTC | TTT | TTC | TTT | TTC | TTC | TTC |
| Gln | CAG | CAA | CAA | CAA | CAG | CAG | CAG | CAA |
| His | CAC | CAC | CAT | CAC | CAT | CAC | CAC | CAC |

3. Exemplary Sequences

In some embodiments, the ORF encoding the RNA-guided DNA binding agent comprises a sequence with at least 93% identity to SEQ ID NO: 311; and/or the ORF has at least 93% identity to SEQ ID NO: 311 over at least its first 50, 200, 250, or 300 nucleotides, or at least 95% identity to SEQ ID NO: 311 over at least its first 30, 50, 70, 100, 150, 200, 250, or 300 nucleotides; and/or the ORF consists of a set of codons of which at least 95%, 96%, 97%, 98%, 99%, 99.5%, or 100% of the codons are codons listed in Table 1; and/or the ORF has an adenine content ranging from its minimum adenine content to 123% of the minimum adenine content; and/or the ORF has an adenine dinucleotide content ranging from its minimum adenine dinucleotide content to 150% of the minimum adenine dinucleotide content.

In some embodiments, the polynucleotide encoding the RNA-guided DNA binding agent comprises a sequence with at least 95%, 96%, 97%, 98%, 99%, 99.5%, or 100% identity to SEQ ID NO: 377.

In some embodiments, the ORF encoding the RNA-guided DNA binding agent comprises a sequence with at least 90% identity to any one of SEQ ID NOs: 201, 204, 207, 209, 210, 211, 212, 214, 215, 217, 218, 220, 221, 223, 224, 226, 227, 229, 230, 250, 252, 254, 265, 266, or 307-375. In some embodiments, the mRNA comprises an ORF encoding an RNA-guided DNA binding agent, wherein the RNA-guided DNA binding agent comprises an amino acid sequence with at least 90% identity to any one of SEQ ID NOs: 203, 206, 208, 213, 216, 219, 222, 225, 228, 268, or 386-396, wherein the ORF has an adenine content ranging from its minimum adenine content to 150% of the minimum adenine content, and/or has a adenine dinucleotide content ranging from its minimum adenine dinucleotide content to 150% of the minimum adenine dinucleotide content. In some embodiments, the encoded RNA-guided DNA binding agent comprises an amino acid sequence with at least 90% identity to any one of SEQ ID NOs: 203, 206, 208, 213, 216, 219, 222, 225, 228, 268, or 386-396, wherein the ORF has a uridine content ranging from its minimum uridine content to 150% of the minimum uridine content, and/or has a uridine dinucleotide content ranging from its minimum uridine dinucleotide content to 150% of the minimum uridine dinucleotide content. In some such embodiments, both the adenine and uridine nucleotide contents are less than or equal to 150% of their respective minima. In some embodiments, both the adenine and uridine dinucleotide contents are less than or equal to 150% of their respective minima. In some embodiments, the mRNA comprises a sequence with at least 90% identity to any one of SEQ ID NOs: 243, 244, 251, 253, 255-261, or 267, wherein the sequence comprises an ORF encoding an RNA-guided DNA binding agent. In some embodiments, the mRNA comprises a sequence with at least 90% identity to any one of SEQ ID NOs: 243, 244, 251, 253, 255-261, or 267, wherein the sequence comprises an ORF encoding an RNA-guided DNA binding agent, wherein the first three nucleotides of SEQ ID NOs: 243, 244, 251, 253, 255-261, or 267 are omitted. In some embodiments, any of the foregoing levels of identity is at least 95%, at least 98%, at least 99%, or 100%.

In some embodiments, the ORF encoding an RNA-guided DNA binding agent has at least 90% identity to any one of SEQ ID NO: 201, 204, 207, 209, 210, 211, 212, 214, 215, 217, 218, 220, 221, 223, 224, 226, 227, 229, 230, 250, 252, 254, 265, 266, or 307-375 over at least its first 30, 50, 70, 100, 150, 200, 250, or 300 nucleotides. The first 30, 50, 70, 100, 150, 200, 250, or 300 nucleotides are measured from the first nucleotide of the start codon (typically ATG), such that the A is nucleotide 1, the T is nucleotide 2, etc. In some embodiments, the open reading frame has at least 90% identity to any one of SEQ ID NO: 201, 204, 207, 209, 210, 211, 212, 214, 215, 217, 218, 220, 221, 223, 224, 226, 227, 229, 230, 250, 252, 254, 265, 266, or 307-375 over at least its first 10%, 12%, 15%, 20%, 25%, 30%, or 35% of its sequence. The length of the sequence of the ORF is the number of nucleotides from the beginning of the start codon to the end of the stop codon, and the first 10%, 12%, 15%, 20%, 25%, 30%, or 35% of its sequence corresponds to the number of nucleotides starting from the first nucleotide of the start codon that make up the indicated percentage of the length of the total sequence.

In some embodiments, the nucleic acid comprising an ORF encoding an RNA-guided DNA binding agent comprises a sequence having at least 90% identity to SEQ ID NO: 243 in which the ORF of SEQ ID NO: 243 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 244 in which the ORF of SEQ ID NO: 244 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 256 in which the ORF of SEQ ID NO: 256 (i.e., SEQ ID NO: 204) is substituted with an alternative ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 257 in which the ORF of SEQ ID NO: 257 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 258 in which the ORF of SEQ ID NO: 258 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 259 in which the ORF of SEQ ID NO: 259 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 260 in which the ORF of SEQ ID NO: 260 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 261 in which the ORF of SEQ ID NO: 261 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 376 in which the ORF of SEQ ID NO: 376 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 377 in which the ORF of SEQ ID NO: 377 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 378 in which the ORF of SEQ ID NO: 378 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 379 in which the ORF of SEQ ID NO: 379 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 380 in which the ORF of SEQ ID NO: 380 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 381 in which the ORF of SEQ ID NO: 381 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 382 in which the ORF of SEQ ID NO: 382 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 383 in which the ORF of SEQ ID NO: 383 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 384 in which the ORF of SEQ ID NO: 384 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the nucleic acid comprising an ORF encoding an RNA-guided DNA binding agent comprises a sequence having at least 90% identity to SEQ ID NO: 385 in which the ORF of SEQ ID NO: 385 (i.e., SEQ ID NO: 204) is substituted with the ORF of any one of SEQ ID NO: 207, 209, 210, 211, 212, 214, 215, 217, 218, 220, 221, 223, 224, 226, 227, 229, 230, 250, 252, 254, 265, 266, or 307-375.

In some embodiments, the degree of identity to the optionally substituted sequences of SEQ ID Nos: 243, 244, 256-61, or 376-385 is at least 95%. In some embodiments, the degree of identity to the optionally substituted sequences of SEQ ID NOs: 243, 244, 256-61, or 376-385 is at least 98%. In some embodiments, the degree of identity to the optionally substituted sequences of SEQ ID NOs: 243, 244, 256-61, or 376-385 is at least 99%. In some embodiments, the degree of identity to the optionally substituted sequences of SEQ ID NOs: 243, 244, 256-61, or 376-385 is 100%.

4. Additional Features of Nucleic Acids, mRNAs, and ORFs

Any of the additional features described herein may be combined to the extent feasible with any of the embodiments described above.

a) Low Uridine Content

In some embodiments, the ORF encoding the RNA-guided DNA-binding agent, e.g. a Cas9 nuclease such as an S. pyogenes Cas9, has a uridine content ranging from its minimum uridine content to about 150% of its minimum uridine content. In some embodiments, the uridine content of the ORF is less than or equal to about 145%, 140%, 135%, 130%, 125%, 120%, 115%, 110%, 105%, 104%, 103%, 102%, or 101% of its minimum uridine content. In some embodiments, the ORF has a uridine content equal to its minimum uridine content. In some embodiments, the ORF has a uridine content less than or equal to about 150% of its minimum uridine content. In some embodiments, the ORF has a uridine content less than or equal to about 145% of its minimum uridine content. In some embodiments, the ORF has a uridine content less than or equal to about 140% of its minimum uridine content. In some embodiments, the ORF has a uridine content less than or equal to about 135% of its minimum uridine content. In some embodiments, the ORF has a uridine content less than or equal to about 130% of its minimum uridine content. In some embodiments, the ORF has a uridine content less than or equal to about 125% of its minimum uridine content. In some embodiments, the ORF has a uridine content less than or equal to about 120% of its minimum uridine content. In some embodiments, the ORF has a uridine content less than or equal to about 115% of its minimum uridine content. In some embodiments, the ORF has a uridine content less than or equal to about 110% of its minimum uridine content. In some embodiments, the ORF has a uridine content less than or equal to about 105% of its minimum uridine content. In some embodiments, the ORF has a uridine content less than or equal to about 104% of its minimum uridine content. In some embodiments, the ORF has a uridine content less than or equal to about 103% of its minimum uridine content. In some embodiments, the ORF has a uridine content less than or equal to about 102% of its minimum uridine content. In some embodiments, the ORF has a uridine content less than or equal to about 101% of its minimum uridine content.

In some embodiments, the ORF has a uridine dinucleotide content ranging from its minimum uridine dinucleotide content to 200% of its minimum uridine dinucleotide content. In some embodiments, the uridine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum uridine dinucleotide content. In some embodiments, the ORF has a uridine dinucleotide content equal to its minimum uridine dinucleotide content. In some embodiments, the ORF has a uridine dinucleotide content less than or equal to about 200% of its minimum uridine dinucleotide content. In some embodiments, the ORF has a uridine dinucleotide content less than or equal to about 195% of its minimum uridine dinucleotide content. In some embodiments, the ORF has a uridine dinucleotide content less than or equal to about 190% of its minimum uridine dinucleotide content. In some embodiments, the ORF has a uridine dinucleotide content less than or equal to about 185% of its minimum uridine dinucleotide content. In some embodiments, the ORF has a uridine dinucleotide content less than or equal to about 180% of its minimum uridine dinucleotide content. In some embodiments, the ORF has a uridine dinucleotide content less than or equal to about 175% of its minimum uridine dinucleotide content. In some embodiments, the ORF has a uridine dinucleotide content less than or equal to about 170% of its minimum uridine dinucleotide content. In some embodiments, the ORF has a uridine dinucleotide content less than or equal to about 165% of its minimum uridine dinucleotide content. In some embodiments, the ORF has a uridine dinucleotide content less than or equal to about 160% of its minimum uridine dinucleotide content. In some embodiments, the ORF has a uridine dinucleotide content less than or equal to about 155% of its minimum uridine dinucleotide content. In some embodiments, the ORF has a uridine dinucleotide content equal to its minimum uridine dinucleotide content. In some embodiments, the ORF has a uridine dinucleotide content less than or equal to about 150% of its minimum uridine dinucleotide content. In some embodiments, the ORF has a uridine dinucleotide content less than or equal to about 145% of its minimum uridine dinucleotide content. In some embodiments, the ORF has a uridine dinucleotide content less than or equal to about 140% of its minimum uridine dinucleotide content. In some embodiments, the ORF has a uridine dinucleotide content less than or equal to about 135% of its minimum uridine dinucleotide content. In some embodiments, the ORF has a uridine dinucleotide content less than or equal to about 130% of its minimum uridine dinucleotide content. In some embodiments, the ORF has a uridine dinucleotide content less than or equal to about 125% of its minimum uridine dinucleotide content. In some embodiments, the ORF has a uridine dinucleotide content less than or equal to about 120% of its minimum uridine dinucleotide content. In some embodiments, the ORF has a uridine dinucleotide content less than or equal to about 115% of its minimum uridine dinucleotide content. In some embodiments, the ORF has a uridine dinucleotide content less than or equal to about 110% of its minimum uridine dinucleotide content. In some embodiments, the ORF has a uridine dinucleotide content less than or equal to about 105% of its minimum uridine dinucleotide content. In some embodiments, the ORF has a uridine dinucleotide content less than or equal to about 104% of its minimum uridine dinucleotide content. In some embodiments, the ORF has a uridine dinucleotide content less than or equal to about 103% of its minimum uridine dinucleotide content. In some embodiments, the ORF has a uridine dinucleotide content less than or equal to about 102% of its minimum uridine dinucleotide content. In some embodiments, the ORF has a uridine dinucleotide content less than or equal to about 101% of its minimum uridine dinucleotide content.

In some embodiments, the ORF has a uridine dinucleotide content ranging from its minimum uridine dinucleotide content to the uridine dinucleotide content that is 90% or lower of the maximum uridine dinucleotide content of a reference sequence that encodes the same protein as the mRNA in question. In some embodiments, the uridine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine dinucleotide content of a reference sequence that encodes the same protein as the mRNA in question.

In some embodiments, the ORF has a uridine trinucleotide content ranging from 0 uridine trinucleotides to 1, 2, 3, 4, 5, 6, 7, 8, 9, 10, 20, 30, 40, or 50 uridine trinucleotides (where a longer run of uridines counts as the number of unique three-uridine segments within it, e.g., a uridine tetranucleotide contains two uridine trinucleotides, a uridine pentanucleotide contains three uridine trinucleotides, etc.). In some embodiments, the ORF has a uridine trinucleotide content ranging from 0% uridine trinucleotides to 0.1%, 0.2%, 0.3%, 0.4%, 0.5%, 0.6%, 0.7%, 0.8%, 0.9%, 1%, 1.5%, or 2% uridine trinucleotides, where the percentage content of uridine trinucleotides is calculated as the percentage of positions in a sequence that are occupied by uridines that form part of a uridine trinucleotide (or longer run of uridines), such that the sequences UUUAAA and UUUUAAAA would each have a uridine trinucleotide content of 50%. For example, in some embodiments, the ORF has a uridine trinucleotide content less than or equal to 2%. For example, in some embodiments, the ORF has a uridine trinucleotide content less than or equal to 1.5%. In some embodiments, the ORF has a uridine trinucleotide content less than or equal to 1%. In some embodiments, the ORF has a uridine trinucleotide content less than or equal to 0.9%. In some embodiments, the ORF has a uridine trinucleotide content less than or equal to 0.8%. In some embodiments, the ORF has a uridine trinucleotide content less than or equal to 0.7%. In some embodiments, the ORF has a uridine trinucleotide content less than or equal to 0.6%. In some embodiments, the ORF has a uridine trinucleotide content less than or equal to 0.5%. In some embodiments, the ORF has a uridine trinucleotide content less than or equal to 0.4%. In some embodiments, the ORF has a uridine trinucleotide content less than or equal to 0.3%. In some embodiments, the ORF has a uridine trinucleotide content less than or equal to 0.2%. In some embodiments, the ORF has a uridine trinucleotide content less than or equal to 0.1%. In some embodiments, the ORF has no uridine trinucleotides.

In some embodiments, the ORF has a uridine trinucleotide content ranging from its minimum uridine trinucleotide content to the uridine trinucleotide content that is 90% or lower of the maximum uridine trinucleotide content of a reference sequence that encodes the same protein as the mRNA in question. In some embodiments, the uridine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine trinucleotide content of a reference sequence that encodes the same protein as the mRNA in question.

A given ORF can be reduced in uridine content or uridine dinucleotide content or uridine trinucleotide content, for example, by using minimal uridine codons in a sufficient fraction of the ORF. For example, an amino acid sequence for an RNA-guided DNA-binding agent can be back-translated into an ORF sequence by converting amino acids to codons, wherein some or all of the ORF uses the exemplary minimal uridine codons shown below. In some embodiments, at least about 50%, 55%, 60%, 65%, 70%, 75%, 80%, 85%, 90%, 95%, 98%, 99%, or 100% of the codons in the ORF are codons listed in Table 6.

TABLE 6
Exemplary minimal uridine codons

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 6.

b) Low Adenine and Uridine Content

To the extent feasible, any of the features described herein with respect to low adenine content can be combined with any of the features described herein with respect to low uridine content. For example, a nucleic acid (e.g., mRNA) may be provided that encodes an RNA-guided DNA-binding agent comprising an ORF having a uridine content ranging from its minimum uridine content to about 150% of its minimum uridine content (e.g., a uridine content of the ORF is less than or equal to about 145%, 140%, 135%, 130%, 125%, 120%, 115%, 110%, 105%, 104%, 103%, 102%, or 101% of its minimum uridine content) and an adenine content ranging from its minimum adenine content to about 150% of its minimum adenine content (e.g., less than or equal to about 145%, 140%, 135%, 130%, 125%, 120%, 115%, 110%, 105%, 104%, 103%, 102%, or 101% of its minimum adenine content). So too for uridine and adenine dinucleotides. Similarly, the content of uridine nucleotides and adenine dinucleotides in the ORF may be as set forth above. Similarly, the content of uridine dinucleotides and adenine nucleotides in the ORF may be as set forth above.

A given ORF can be reduced in uridine and adenine nucleotide and/or dinucleotide content, for example, by using minimal uridine and adenine codons in a sufficient fraction of the ORF. For example, an amino acid sequence for an RNA-guided DNA-binding agent can be back-translated into an ORF sequence by converting amino acids to codons, wherein some or all of the ORF uses the exemplary minimal uridine and adenine codons shown below. In some embodiments, at least about 50%, 55%, 60%, 65%, 70%, 75%, 80%, 85%, 90%, 95%, 98%, 99%, or 100% of the codons in the ORF are codons listed in Table 7.

TABLE 7
Exemplary minimal uridine and adenine codons

| | Amino Acid | Minimal uridine codon |
|---|---|---|
| A | Alanine | GCC or GCG |
| G | Glycine | GGC or GGG |
| V | Valine | GUC or GUG |
| D | Aspartic acid | GAC |
| E | Glutamic acid | GAG |
| I | Isoleucine | AUC |
| T | Threonine | ACC or ACG |
| N | Asparagine | AAC |
| K | Lysine | AAG |
| S | Serine | AGC or UCC or UCG |
| R | Arginine | CGC or CGG |
| L | Leucine | CUG or CUC |
| P | Proline | CCG or CCC |
| H | Histidine | CAC |
| Q | Glutamine | CAG |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 7. As can be seen in Table 7, each of the three listed serine codons contains either one A or one U. In some embodiments, uridine minimization is prioritized by using AGC codons for serine. In some embodiments, adenine minimization is prioritized by using UCC and/or UCG codons for serine.

c) UTRs; Kozak Sequences

In some embodiments, the polynucleotide (e.g., mRNA) comprises a 5′ UTR, a 3′ UTR, or 5′ and 3′ UTRs. In some embodiments, the polynucleotide (e.g., mRNA) comprises at least one UTR from Hydroxysteroid 17-Beta Dehydrogenase 4 (HSD17B4 or HSD), e.g., a 5′ UTR from HSD. In some embodiments, the polynucleotide (e.g., mRNA) comprises at least one UTR from a globin polynucleotide (e.g., mRNA), for example, human alpha globin (HBA) polynucleotide (e.g., mRNA), human beta globin (HBB) polynucleotide (e.g., mRNA), or Xenopus laevis beta globin (XBG) polynucleotide (e.g., mRNA). In some embodiments, the polynucleotide (e.g., mRNA) comprises a 5′ UTR, 3′ UTR, or 5′ and 3′ UTRs from a globin polynucleotide (e.g., mRNA), such as HBA, HBB, or XBG. In some embodiments, the polynucleotide (e.g., mRNA) comprises a 5′ UTR from bovine growth hormone, cytomegalovirus (CMV), mouse Hba-a1, HSD, an albumin gene, HBA, HBB, or XBG. In some embodiments, the polynucleotide (e.g., mRNA) comprises a 3′ UTR from bovine growth hormone, cytomegalovirus, mouse Hba-a1, HSD, an albumin gene, HBA, HBB, or XBG. In some embodiments, the polynucleotide (e.g., mRNA) comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the polynucleotide (e.g., mRNA) comprises 5′ and 3′ UTRs that are from the same source, e.g., a constitutively expressed polynucleotide (e.g., mRNA) such as actin, albumin, or a globin such as HBA, HBB, or XBG.

In some embodiments, a nucleic acid disclosed herein comprises a 5′ UTR with at least 90% identity to any one of SEQ ID NOs: 232, 234, 236, 238, 241, or 275-277. In some embodiments, a nucleic acid disclosed herein comprises a 3′ UTR with at least 90% identity to any one of SEQ ID NOs: 233, 235, 237, 239, or 240. In some embodiments, any of the foregoing levels of identity is at least 95%, at least 98%, at least 99%, or 100%. In some embodiments, a nucleic acid disclosed herein comprises a 5′ UTR having the sequence of any one of SEQ ID NOs: 232, 234, 236, 238, or 241. In some embodiments, a nucleic acid disclosed herein comprises a 3′ UTR having the sequence of any one of SEQ ID NOs: 233, 235, 237, 239, or 240.

In some embodiments, the polynucleotide (e.g., mRNA) does not comprise a 5′ UTR, e.g., there are no additional nucleotides between the 5′ cap and the start codon. In some embodiments, the polynucleotide (e.g., mRNA) comprises a Kozak sequence (described below) between the 5′ cap and the start codon, but does not have any additional 5′ UTR. In some embodiments, the polynucleotide (e.g., mRNA) does not comprise a 3′ UTR, e.g., there are no additional nucleotides between the stop codon and the poly-A tail.

In some embodiments, the polynucleotide (e.g., mRNA) comprises a Kozak sequence. The Kozak sequence can affect translation initiation and the overall yield of a polypeptide translated from a nucleic acid. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase. In some embodiments, the Kozak sequence is rccAUGg with zero mismatches or with up to one or two mismatches to positions in lowercase. In some embodiments, the Kozak sequence is gccRccAUGG (nucleotides 4-13 of SEQ ID NO: 305) with zero mismatches or with up to one, two, or three mismatches to positions in lowercase. In some embodiments, the Kozak sequence is gccAccAUG with zero mismatches or with up to one, two, three, or four mismatches to positions in lowercase. In some embodiments, the Kozak sequence is GCCACCAUG. In some embodiments, the Kozak sequence is gccgccRccAUGG (SEQ ID NO: 305) with zero mismatches or with up to one, two, three, or four mismatches to positions in lowercase.

d) Poly-A Tail

In some embodiments, the polynucleotide (e.g., mRNA) further comprises a polyadenylated (poly-A) tail. In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the polynucleotide (e.g., mRNA) described herein may comprise consecutive adenine nucleotides located 3′ to nucleotides encoding an RNA-guided DNA-binding agent or a sequence of interest. In some instances, the poly-A tails on polynucleotide (e.g., mRNA) comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after at least 8-50 consecutive adenine nucleotides. In some embodiments, the one or more non-adenine nucleotide is located after at least 8-100 consecutive adenine nucleotides. In some embodiments, the non-adenine nucleotide is after one, two, three, four, five, six, or seven adenine nucleotides and is followed by at least 8 consecutive adenine nucleotides.

The poly-A tail of the present disclosure may comprise one sequence of consecutive adenine nucleotides followed by one or more non-adenine nucleotides, optionally followed by additional adenine nucleotides.

In some embodiments, the poly-A tail comprises or contains one non-adenine nucleotide or one consecutive stretch of 2-10 non-adenine nucleotides. In some embodiments, the non-adenine nucleotide(s) is located after at least 8, 9, 10, 11, or 12 consecutive adenine nucleotides. In some instances, the one or more non-adenine nucleotides are located after at least 8-50 consecutive adenine nucleotides. In some embodiments, the one or more non-adenine nucleotides are located after at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive adenine nucleotides.

In some embodiments, the non-adenine nucleotide is guanine, cytosine, or thymine. In some instances, the non-adenine nucleotide is a guanine nucleotide. In some embodiments, the non-adenine nucleotide is a cytosine nucleotide. In some embodiments, the non-adenine nucleotide is a thymine nucleotide. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides. An exemplary poly-A tail comprising non-adenine nucleotides is provided as SEQ ID NO: 262.

e) Modified Nucleotides

In some embodiments, the nucleic acid comprising an ORF encoding an RNA-guided DNA-binding agent comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof. In some embodiments the modified uridine is 5-methoxyuridine. In some embodiments the modified uridine is 5-iodouridine. In some embodiments the modified uridine is pseudouridine. In some embodiments the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in the nucleic acid are modified uridines. In some embodiments, 10%-25%, 15-25%, 25-35%, 35-45%, 45-55%, 55-65%, 65-75%, 75-85%, 85-95%, or 90-100% of the uridine positions in the nucleic acid are modified uridines, e.g., 5-methoxyuridine, 5-iodouridine, N1-methyl pseudouridine, pseudouridine, or a combination thereof. In some embodiments, 10%-25%, 15-25%, 25-35%, 35-45%, 45-55%, 55-65%, 65-75%, 75-85%, 85-95%, or 90-100% of the uridine positions in the nucleic acid are 5-methoxyuridine. In some embodiments, 10%-25%, 15-25%, 25-35%, 35-45%, 45-55%, 55-65%, 65-75%, 75-85%, 85-95%, or 90-100% of the uridine positions in the nucleic acid are pseudouridine. In some embodiments, 10%-25%, 15-25%, 25-35%, 35-45%, 45-55%, 55-65%, 65-75%, 75-85%, 85-95%, or 90-100% of the uridine positions in the nucleic acid are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in the nucleic acid are 5-iodouridine. In some embodiments, 10%-25%, 15-25%, 25-35%, 35-45%, 45-55%, 55-65%, 65-75%, 75-85%, 85-95%, or 90-100% of the uridine positions in the nucleic acid are 5-methoxyuridine, and the remainder are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in the nucleic acid are 5-iodouridine, and the remainder are N1-methyl pseudouridine.

f) 5′ Cap

In some embodiments, the nucleic acid (e.g., mRNA) comprising an ORF encoding an RNA-guided DNA-binding agent comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acid with a cap other than Cap1 or Cap2, potentially inhibiting translation of the mRNA.

A cap can be included in an RNA co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’cap analogs 7-methyl(3′-O-methyl)GpppG and 7-methyl(3′deoxy)GpppG,” RNA 7: 1486-1495. The ARCA structure is shown below.

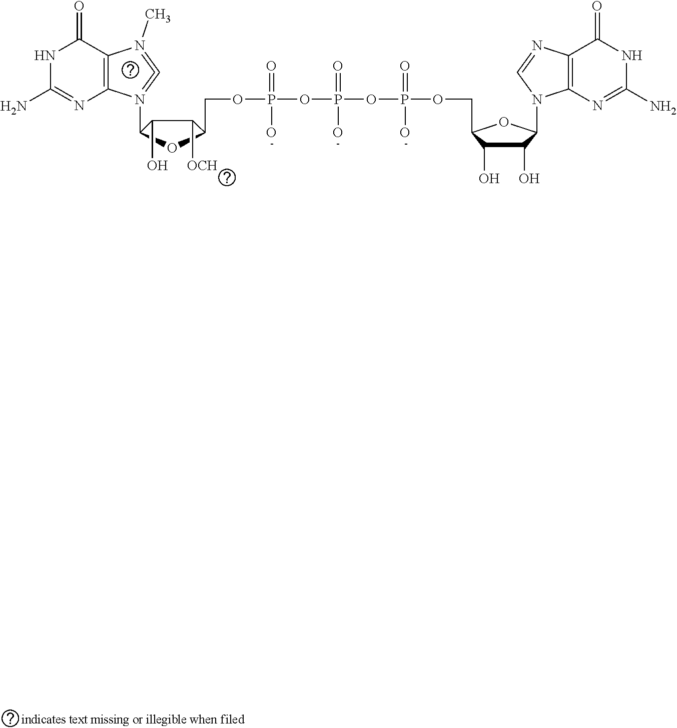

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap AG™ and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

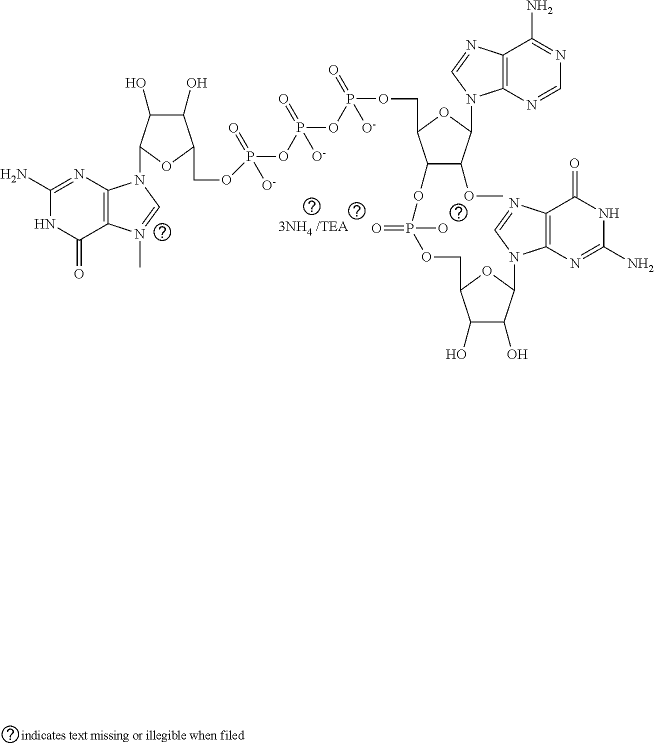

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-A130.

F. Determination of Efficacy of RNAs

In some embodiments, the efficacy of a gRNA is determined when delivered together with other components, e.g., a nucleic acid encoding an RNA-guided DNA binding agent such as any of those described herein. In some embodiments, the efficacy of a combination of a corticosteroid and a gRNA, and optionally an RNA-guided DNA binding agent or nucleic acid encoding such an agent is determined.

As described herein, use of an RNA-guided DNA binding agent and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs, compositions, or treatments comprising administering a gRNA, corticosteroid, and optionally an RNA-guided DNA binding agent or nucleic acid encoding such an agent is determined based on in vitro models. In some embodiments, the in vitro model is HEK293 cells. In some embodiments, the in vitro model is HUH7 human hepatocarcinoma cells. In some embodiments, the in vitro model is HepG2 cells. In some embodiments, the in vitro model is primary human hepatocytes. In some embodiments, the in vitro model is primary cynomolgus hepatocytes. With respect to using primary human hepatocytes, commercially available primary human hepatocytes can be used to provide greater consistency between experiments. In some embodiments, the number of off-target sites at which a deletion or insertion occurs in an in vitro model (e.g., in primary human hepatocytes) is determined, e.g., by analyzing genomic DNA from primary human hepatocytes transfected in vitro with Cas9 mRNA and the guide RNA. In some embodiments, such a determination comprises analyzing genomic DNA from primary human hepatocytes transfected in vitro with Cas9 mRNA, the guide RNA, and a donor oligonucleotide. Exemplary procedures for such determinations are provided in the working examples below.

In some embodiments, the efficacy of particular gRNAs, compositions, or treatments comprising administering a gRNA, corticosteroid, and optionally an RNA-guided DNA binding agent or nucleic acid encoding such an agent is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs, compositions, or treatments comprising administering a gRNA, corticosteroid, and optionally an RNA-guided DNA binding agent or nucleic acid encoding such an agent is determined based on in vivo models. In some embodiments, the in vivo model is a rodent model. In some embodiments, the rodent model is a mouse which expresses a human TTR gene, which may be a mutant human TTR gene. In some embodiments, the in vivo model is a non-human primate, for example cynomolgus monkey.

In some embodiments, the efficacy of a guide RNA, compositions, or treatments comprising administering a gRNA, corticosteroid, and optionally an RNA-guided DNA binding agent or nucleic acid encoding such an agent is measured by percent editing of TTR. In some embodiments, the percent editing of TTR is compared to the percent editing necessary to achieve knockdown of TTR protein, e.g., in the cell culture media in the case of an in vitro model or in serum or tissue in the case of an in vivo model.

In some embodiments, the efficacy of a gRNA, compositions, or treatments comprising administering a gRNA, corticosteroid, and optionally an RNA-guided DNA binding agent or nucleic acid encoding such an agent is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a hepatocyte), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., hepatocyte). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method), as described in WO2018/067447 or Schmidt et al., Nature Methods 4:1051-1057 (2007).

In some embodiments, the method comprises isolating cellular DNA from a cell that has been induced to have a double strand break (DSB) and optionally that has been provided with an HDR template to repair the DSB; performing at least one cycle of linear amplification of the DNA with a tagged primer; isolating the linear amplification products that comprise tag, thereby discarding any amplification product that was amplified with a non-tagged primer; optionally further amplifying the isolated products; and analyzing the linear amplification products, or the further amplified products, to determine the presence or absence of an editing event such as, for example, a double strand break, an insertion, deletion, or HDR template sequence in the target DNA. In some instances, the editing event can be quantified. Quantification and the like as used herein (including in the context of HDR and non-HDR editing events such as indels) includes detecting the frequency and/or type(s) of editing events in a population.

In some embodiments, only one cycle of linear amplification is conducted.

In some instances, the tagged primer comprises a molecular barcode. In some embodiments, the tagged primer comprises a molecular barcode, and only one cycle of linear amplification is conducted.

In some embodiments, detecting gene editing events, such as the formation of insertion/deletion (“indel”) mutations and homology directed repair (HDR) events in target DNA, further comprises sequencing the linear amplified products or the further amplified products. Sequencing may comprise any method known to those of skill in the art, including, next generation sequencing, and cloning the linear amplification products or further amplified products into a plasmid and sequencing the plasmid or a portion of the plasmid. Exemplary next generation sequencing methods are discussed, e.g., in Shendure et al., Nature 26:1135-1145 (2008). In other aspects, detecting gene editing events, such as the formation of insertion/deletion (“indel”) mutations and homology directed repair (HDR) events in target DNA, further comprises performing digital PCR (dPCR) or droplet digital PCR (ddPCR) on the linear amplified products or the further amplified products or contacting the linear amplified products or the further amplified products with a nucleic acid probe designed to identify DNA comprising HDR template sequence and detecting the probes that have bound to the linear amplified product(s) or further amplified product(s). In some embodiments, the method further comprises determining the location of the HDR template in the target DNA.

In certain embodiments, the method further comprises determining the sequence of an insertion site in the target DNA, wherein the insertion site is the location where the HDR template incorporates into the target DNA, and wherein the insertion site may include some target DNA sequence and some HDR template sequence.

In some embodiments, the efficacy of a guide RNA or combination is measured by secretion of TTR. In some embodiments, secretion of TTR is measured using an enzyme-linked immunosorbent assay (ELISA) assay with cell culture media or serum. In some embodiments, secretion of TTR is measured in the same in vitro or in vivo systems or models used to measure editing. In some embodiments, secretion of TTR is measured in primary human hepatocytes. In some embodiments, secretion of TTR is measured in HUH7 cells. In some embodiments, secretion of TTR is measured in HepG2 cells.

ELISA assays are generally known to the skilled artisan and can be designed to determine serum TTR levels. In one exemplary embodiment, blood is collected and the serum is isolated. The total TTR serum levels may be determined using a Mouse Prealbumin (Transthyretin) ELISA Kit (Aviva Systems Biology, Cat. OKIA00111) or similar kit for measuring human TTR. If no kit is available, an ELISA can be developed using plates that are pre-coated with capture antibody specific for the TTR one is measuring. The plate is next incubated at room temperature for a period of time before washing. Enzyme-anti-TTR antibody conjugate is added and incubated. Unbound antibody conjugate is removed and the plate washed before the addition of the chromogenic substrate solution that reacts with the enzyme. The plate is read on an appropriate plate reader at an absorbance specific for the enzyme and substrate used.

In some embodiments, the amount of TTR in cells (including those from tissue) measures efficacy of a gRNA or combination. In some embodiments, the amount of TTR in cells is measured using western blot. In some embodiments, the cell used is HUH17 cells. In some embodiments, the cell used is a primary human hepatocyte. In some embodiments, the cell used is a primar cell obtained from an animal. In some embodiments, the amount of TTR is compared to the amount of glyceraldehyde 3-phosphate dehydrogenase GAPDH (a housekeeping gene) to control for changes in cell number.

### III. LNP Formulations and Treatment of ATTR

In some embodiments, a method of treating ATTR is provided comprising administering a corticosteroid and a composition comprising a guide RNA as described herein, e.g., comprising any one or more of the guide sequences of SEQ ID NOs: 5-82, or any one or more of the sgRNAs of SEQ ID Nos: 87-124. In some embodiments, gRNAs comprising any one or more of the guide sequences of SEQ ID NOs: 5-82, or any one or more of the sgRNAs of SEQ ID Nos: 87-124 are administered to treat ATTR. The guide RNA may be administered together with an RNA-guided DNA nuclease such as a Cas nuclease (e.g., Cas9) or a nucleic acid or vector described herein encoding an RNA-guided DNA nuclease. In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of treating ATTR is provided comprising administering a corticosteroid and a composition comprising a guide RNA as described herein, e.g., comprising any one or more of the guide sequences of SEQ ID NOs: 5-72, 74-78, and 80-82, or any one or more of the sgRNAs of SEQ ID Nos: 87-113, 115-120, and 122-124. In some embodiments, gRNAs comprising any one or more of the guide sequences of SEQ ID NOs: 5-72, 74-78, and 80-82, or any one or more of the sgRNAs of SEQ ID Nos: 87-113, 115-120, and 122-124 are administered to treat ATTR. The guide RNA is optionally administered together with an RNA-guided DNA nuclease such as a Cas nuclease (e.g., Cas9) or a nucleic acid or vector described herein encoding an RNA-guided DNA nuclease. In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of reducing TTR serum concentration is provided comprising administering a corticosteroid and a guide RNA as described herein, e.g., comprising any one or more of the guide sequences of SEQ ID NOs: 5-82, or any one or more of the sgRNAs of SEQ ID Nos: 87-124. In some embodiments, gRNAs comprising any one or more of the guide sequences of SEQ ID NOs: 5-82 or any one or more of the sgRNAs of SEQ ID Nos: 87-124 are administered to reduce or prevent the accumulation of TTR in amyloids or amyloid fibrils. The gRNA is administered together with a nucleic acid or vector described herein encoding an RNA-guided DNA nuclease such as a Cas nuclease (e.g., Cas9). In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of reducing TTR serum concentration is provided comprising administering a guide RNA as described herein, e.g., comprising any one or more of the guide sequences of SEQ ID NOs: 5-72, 74-78, and 80-82, or any one or more of the sgRNAs of SEQ ID Nos: 87-113, 115-120, and 122-124. In some embodiments, gRNAs comprising any one or more of the guide sequences of SEQ ID NOs: 5-72, 74-78, and 80-82, or any one or more of the sgRNAs of SEQ ID Nos: 87-113, 115-120, and 122-124 are administered to reduce or prevent the accumulation of TTR in amyloids or amyloid fibrils. The guide RNA is optionally administered together with an RNA-guided DNA nuclease such as a Cas nuclease (e.g., Cas9) or a nucleic acid or vector described herein encoding an RNA-guided DNA nuclease. In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG) and optionally a neutral lipid (e.g., DSPC).

In some embodiments, a method of reducing or preventing the accumulation of TTR in amyloids or amyloid fibrils of a subject is provided comprising administering a corticosteroid and a composition comprising a guide RNA as described herein, e.g., comprising any one or more of the guide sequences of SEQ ID NOs: 5-82, or any one or more of the sgRNAs of SEQ ID Nos: 87-124. In some embodiments, a method of reducing or preventing the accumulation of TTR in amyloids or amyloid fibrils of a subject is provided comprising administering a corticosteroid and a composition comprising any one or more of the sgRNAs of SEQ ID Nos: 87-113. In some embodiments, gRNAs comprising any one or more of the guide sequences of SEQ ID NOs: 5-82 or any one or more of the sgRNAs of SEQ ID Nos: 87-124 are administered to reduce or prevent the accumulation of TTR in amyloids or amyloid fibrils. The gRNA is optionally administered together with a nucleic acid or vector described herein encoding an RNA-guided DNA nuclease such as a Cas nuclease (e.g., Cas9). In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of reducing or preventing the accumulation of TTR in amyloids or amyloid fibrils of a subject is provided comprising administering a composition comprising a guide RNA as described herein, e.g., comprising any one or more of the guide sequences of SEQ ID NOs: 5-72, 74-78, and 80-82, or any one or more of the sgRNAs of SEQ ID Nos: 87-124. In some embodiments, a method of reducing or preventing the accumulation of TTR in amyloids or amyloid fibrils of a subject is provided comprising administering a composition comprising any one or more of the sgRNAs of SEQ ID Nos: 87-113, 115-120, and 122-124. In some embodiments, gRNAs comprising any one or more of the guide sequences of SEQ ID NOs: 5-72, 74-78, and 80-82 or any one or more of the sgRNAs of SEQ ID Nos: 87-113, 115-120, and 122-124 are administered to reduce or prevent the accumulation of TTR in amyloids or amyloid fibrils. The guide RNA is optionally administered together with an RNA-guided DNA nuclease such as a Cas nuclease (e.g., Cas9) or a nucleic acid or vector described herein encoding an RNA-guided DNA nuclease. In some embodiments, the RNA-guided DNA nuclease is a Cas cleavase. In some embodiments, the RNA-guided DNA nuclease is a Cas from a Type-II CRISPR/Cas system. In some embodiments, the RNA-guided DNA nuclease is a Cas9. In some embodiments, the RNA-guided DNA nuclease is an S. pyogenes Cas9 nuclease. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the gRNA comprising a guide sequence of Table 1 or one or more sgRNAs from Table 2 together with an RNA-guided DNA nuclease such as a Cas nuclease translated from the nucleic acid induce DSBs, and non-homologous ending joining (NHEJ) during repair leads to a mutation in the TTR gene. In some embodiments, NHEJ leads to a deletion or insertion of a nucleotide(s), which induces a frame shift or nonsense mutation in the TTR gene.

In some embodiments, administering the corticosteroid and the guide RNA (and optionally an RNA-guided DNA binding agent or a nucleic acid encoding an RNA-guided DNA binding agent) (e.g., in a composition provided herein) reduces levels (e.g., serum levels) of TTR in the subject, and therefore prevents accumulation and aggregation of TTR in amyloids or amyloid fibrils.

In some embodiments, reducing or preventing the accumulation of TTR in amyloids or amyloid fibrils of a subject comprises reducing or preventing TTR deposition in one or more tissues of the subject, such as stomach, colon, or nervous tissue. In some embodiments, the nervous tissue comprises sciatic nerve or dorsal root ganglion. In some embodiments, TTR deposition is reduced in two, three, or four of the stomach, colon, dorsal root ganglion, and sciatic nerve. The level of deposition in a given tissue can be determined using a biopsy sample, e.g., using immunostaining. In some embodiments, reducing or preventing the accumulation of TTR in amyloids or amyloid fibrils of a subject and/or reducing or preventing TTR deposition is inferred based on reducing serum TTR levels for a period of time. As discussed in the examples, it has been found that reducing serum TTR levels in accordance with methods and uses provided herein can result in clearance of deposited TTR from tissues such as those discussed above and in the examples, e.g., as measured 8 weeks after administration of the composition.

In some embodiments, the subject is mammalian. In some embodiments, the subject is human. In some embodiments, the subject is cow, pig, monkey, sheep, dog, cat, fish, or poultry.

In some embodiments, the use of one or more guide RNAs as described herein, e.g., comprising any one or more of the guide sequences in Table 1 or one or more sgRNAs from Table 2 (e.g., in a composition provided herein) and of a nucleic acid (e.g., mRNA) described herein encoding an RNA-guided DNA-binding agent is provided for the preparation of a medicament for treating a human subject having ATTR. The RNA-guided DNA-binding agent may be a Cas9, e.g. an S. pyogenes Cas9. In particular embodiments, the guide RNA is chemically modified.

In some embodiments, the composition comprising the guide RNA and nucleic acid is administered intravenously. In some embodiments, the composition comprising the guide RNA and nucleic acid is administered into the hepatic circulation.

In some embodiments, a single administration of a composition comprising a guide RNA (and optionally an RNA-guided DNA binding agent or a nucleic acid encoding an RNA-guided DNA binding agent) provided herein is sufficient to knock down expression of the mutant protein. In some embodiments, a single administration of a composition comprising a guide RNA (and optionally an RNA-guided DNA binding agent or a nucleic acid encoding an RNA-guided DNA binding agent) provided herein is sufficient to knock out expression of the mutant protein in a population of cells. In other embodiments, more than one administration of a composition comprising a guide RNA (and optionally an RNA-guided DNA binding agent or a nucleic acid encoding an RNA-guided DNA binding agent) provided herein may be beneficial to maximize editing via cumulative effects. For example, a composition provided herein can be administered 2, 3, 4, 5, or more times, such as 2 times. Administrations can be separated by a period of time ranging from, e.g., 1 day to 2 years, such as 1 to 7 days, 7 to 14 days, 14 days to 30 days, 30 days to 60 days, 60 days to 120 days, 120 days to 183 days, 183 days to 274 days, 274 days to 366 days, or 366 days to 2 years.

In some embodiments, a composition is administered in an effective amount in the range of 0.01 to 10 mg/kg (mpk), e.g., 0.01 to 0.1 mpk, 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, or 10 mpk. In some embodiments, a composition is administered in the amount of 2-4 mpk, such as 2.5-3.5 mpk. In some embodiments, a composition is administered in the amount of about 3 mpk. As reported herein, for an LNP composition, the dosage or effective amount is assessed by total RNA administered.

In some embodiments, the efficacy of treatment with the compositions of the invention is seen at 1 year, 2 years, 3 years, 4 years, 5 years, or 10 years after delivery. In some embodiments, efficacy of treatment with the compositions of the invention is assessed by measuring serum levels of TTR before and after treatment. In some embodiments, efficacy of treatment with the compositions assessed via a reduction of serum levels of TTR is seen at 1 week, 2 weeks, 3 weeks, 4 weeks, 2 months, 3 months, 4 months, 5 months, 6 months, 7 months, 8 months, 9 months, 10 months, or at 11 months.

In some embodiments, treatment slows or halts disease progression.

In some embodiments, treatment slows or halts progression of FAP. In some embodiments, treatment results in improvement, stabilization, or slowing of change in symptoms of sensorimotor neuropathy or autonomic neuropathy.

In some embodiments, treatment results in improvement, stabilization, or slowing of change in symptoms of FAC. In some embodiments, treatment results in improvement, stabilization, or slowing of change symptoms of restrictive cardiomyopathy or congestive heart failure.

In some embodiments, efficacy of treatment is measured by increased survival time of the subject. In some embodiments, efficacy of treatment is measured by increased tolerability of the treatment. In some embodiments, increased tolerability, e.g. cytokine, complement, or other immune response is measured.

In some embodiments, efficacy of treatment is measured by improvement or slowing of progression in symptoms of sensorimotor or autonomic neuropathy. In some embodiments, efficacy of treatment is measured by an increase or a slowing of decrease in ability to move an area of the body or to feel in any area of the body. In some embodiments, efficacy of treatment is measured by improvement or a slowing of decrease in the ability to swallow; breath; use arms, hands, legs, or feet; or walk. In some embodiments, efficacy of treatment is measured by improvement or a slowing of progression of neuralgia. In some embodiments, the neuralgia is characterized by pain, burning, tingling, or abnormal feeling. In some embodiments, efficacy of treatment is measured by improvement or a slowing of increase in postural hypotension, dizziness, gastrointestinal dysmotility, bladder dysfunction, or sexual dysfunction. In some embodiments, efficacy of treatment is measured by improvement or a slowing of progression of weakness. In some embodiments, efficacy of treatment is measured using electromyogram, nerve conduction tests, or patient-reported outcomes.

In some embodiments, efficacy of treatment is measured by improvement or slowing of progression of symptoms of congestive heart failure or CHF. In some embodiments, efficacy of treatment is measured by an decrease or a slowing of increase in shortness of breath, trouble breathing, fatigue, or swelling in the ankles, feet, legs, abdomen, or veins in the neck. In some embodiments, efficacy of treatment is measured by improvement or a slowing of progression of fluid buildup in the body, which may be assessed by measures such as weight gain, frequent urination, or nighttime cough. In some embodiments, efficacy of treatment is measured using cardiac biomarker tests (such as B-type natriuretic peptide [BNP] or N-terminal pro b-type natriuretic peptide [NT-proBNP]), lung function tests, chest x-rays, or electrocardiography.

A. Combination Therapy

In some embodiments, the invention comprises combination therapies comprising administering a corticosteroid and any one of the gRNAs comprising any one or more of the guide sequences disclosed in Table 1 or any one or more of the sgRNAs in Table 2 (and optionally an RNA-guided DNA binding agent or a nucleic acid described herein encoding an RNA-guided DNA binding agent, such as a nucleic acid (e.g. mRNA) or vector described herein encoding an S. pyogenes Cas9) (e.g., in a composition provided herein) together with an additional therapy suitable for alleviating symptoms of ATTR. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the additional therapy for ATTR is a treatment for sensorimotor or autonomic neuropathy. In some embodiments, the treatment for sensorimotor or autonomic neuropathy is a nonsteroidal anti-inflammatory drug, antidepressant, anticonvulsant medication, antiarrythmic medication, or narcotic agent. In some embodiments, the antidepressant is a tricylic agent or a serotonin-norepinephrine reuptake inhibitor. In some embodiments, the antidepressant is amitriptyline, duloxetine, or venlafaxine. In some embodiments, the anticonvulsant agent is gabapentin, pregabalin, topiramate, or carbamazepine. In some embodiments, the additional therapy for sensorimotor neuropathy is transcutaneous electrical nerve stimulation.

In some embodiments, the additional therapy for ATTR is a treatment for restrictive cardiomyopathy or congestive heart failure (CHF). In some embodiments, the treatment for CHF is a ACE inhibitor, aldosterone antagonist, angiotensin receptor blocker, beta blocker, digoxin, diuretic, or isosorbide dinitrate/hydralazine hydrochloride. In some embodiments, the ACE inhibitor is enalapril, captopril, ramipril, perindopril, imidapril, or quinapril. In some embodiments, the aldosterone antagonist is eplerenone or spironolactone. In some embodiments, the angiotensin receptor blocker is azilsartan, cadesartan, eprosartan, irbesartan, losartan, olmesartan, telmisartan, or valsartan. In some embodiments, the beta blocker is acebutolol, atenolol, bisoprolol, metoprolol, nadolol, nebivolol, or propranolol. In some embodiments, the diuretic is chlorothiazide, chlorthalidone, hydrochlorothiazide, indapamide, metolazone, bumetanide, furosemide, torsemide, amiloride, or triameterene.

In some embodiments, the combination therapy comprises administering a corticosteroid and any one of the gRNAs comprising any one or more of the guide sequences disclosed in Table 1 or any one or more of the sgRNAs in Table 2 (and optionally an RNA-guided DNA binding agent or a nucleic acid described herein encoding an RNA-guided DNA binding agent) (e.g., in a composition provided herein) together with a siRNA that targets TTR or mutant TTR. In some embodiments, the siRNA is any siRNA capable of further reducing or eliminating the expression of wild type or mutant TTR. In some embodiments, the siRNA is the drug Patisiran (ALN-TTR02) or ALN-TTRsc02. In some embodiments, the siRNA is administered after any one of the gRNAs comprising any one or more of the guide sequences disclosed in Table 1 or any one or more of the sgRNAs in Table 2 (e.g., in a composition provided herein). In some embodiments, the siRNA is administered on a regular basis following treatment with any of the gRNA compositions provided herein.

In some embodiments, the combination therapy comprises administering a corticosteroid and any one of the gRNAs comprising any one or more of the guide sequences disclosed in Table 1 or any one or more of the sgRNAs in Table 2 (and optionally an RNA-guided DNA binding agent or a nucleic acid described herein encoding an RNA-guided DNA binding agent) (e.g., in a composition provided herein) together with antisense nucleotide that targets TTR or mutant TTR. In some embodiments, the antisense nucleotide is any antisense nucleotide capable of further reducing or eliminating the expression of wild type or mutant TTR. In some embodiments, the antisense nucleotide is the drug Inotersen (IONS-TTR_{Rx}). In some embodiments, the antisense nucleotide is administered after any one of the gRNAs comprising any one or more of the guide sequences disclosed in Table 1 or any one or more of the sgRNAs in Table 2 and a nucleic acid encoding an RNA-guided DNA-binding agent (e.g., in a composition provided herein). In some embodiments, the antisense nucleotide is administered on a regular basis following treatment with any of the gRNA compositions provided herein.

In some embodiments, the combination therapy comprises administering a corticosteroid and any one of the gRNAs comprising any one or more of the guide sequences disclosed in Table 1 or any one or more of the sgRNAs in Table 2 (and optionally an RNA-guided DNA binding agent or a nucleic acid described herein encoding an RNA-guided DNA binding agent) (e.g., in a composition provided herein) together with a small molecule stabilizer that promotes kinetic stabilization of the correctly folded tetrameric form of TTR. In some embodiments, the small molecule stabilizer is the drug tafamidis (Vyndaqel®) or diflunisal. In some embodiments, the small molecule stabilizer is administered after any one of the gRNAs comprising any one or more of the guide sequences disclosed in Table 1 or any one or more of the sgRNAs in Table 2 (e.g., in a composition provided herein). In some embodiments, the small molecule stabilizer is administered on a regular basis following treatment with any of the compositions provided herein.

In any of the foregoing embodiments, the guide sequences disclosed in Table 1 may be selected from SEQ ID NOs: 5-72, 74-78, and 80-82, and/or the sgRNAs in Table 2 may be selected from SEQ ID Nos: 87-113, 115-120, and 122-124, and/or the guide RNA may be a chemically modified guide RNA.

B. Delivery of Nucleic Acid Compositions

In some embodiments, the nucleic acid compositions described herein, comprising a gRNA, and optionally a nucleic acid described herein encoding an RNA-guided DNA-binding agent as RNA or encoded on one or more vectors, are formulated in or administered via a lipid nanoparticle; see e.g., WO2017173054A1 published Oct. 5, 2017 and WO2019067992A1 published Apr. 4, 2019, the contents of which are hereby incorporated by reference in their entirety. Any lipid nanoparticle (LNP) known to those of skill in the art to be capable of delivering nucleotides to subjects may be utilized with the guide RNAs described herein, and optionally the nucleic acid encoding an RNA-guided DNA nuclease.

Disclosed herein are various embodiments of LNP formulations for RNAs, including CRISPR/Cas cargoes. Such LNP formulations may include (i) a CCD lipid, such as an amine lipid, (ii) a neutral lipid, (iii) a helper lipid, and (iv) a stealth lipid, such as a PEG lipid. Some embodiments of the LNP formulations include an “amine lipid”, along with a helper lipid, a neutral lipid, and a stealth lipid such as a PEG lipid. In some embodiments, the LNP formulations include less than 1 percent neutral phospholipid. In some embodiments, the LNP formulations include less than 0.5 percent neutral phospholipid. By “lipid nanoparticle” is meant a particle that comprises a plurality of (i.e. more than one) lipid molecules physically associated with each other by intermolecular forces.

CCD Lipids

Lipid compositions for delivery of CRISPR/Cas mRNA and guide RNA components to a target cell, such as a liver cell comprise a CCD Lipid.

In some embodiments, the CCD lipid is Lipid A, which is (9Z,12Z)-3-((4,4-bis(octyloxy)butanoyDoxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

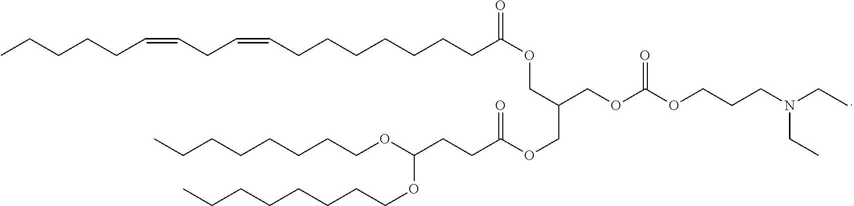 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86).

In some embodiments, the CCD lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl) bis(decanoate). Lipid B can be depicted as:

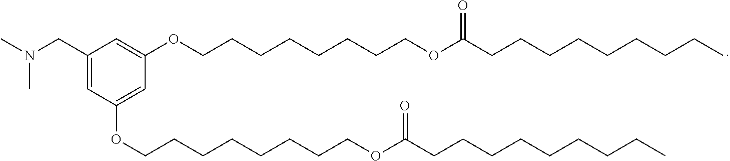 [SMILES: CCCCCCCCCC(=O)OCCCCCCCCOc1cc(CN(C)C)cc(OCCCCCCCCOC(=O)CCCCCCCCC)c1]

Lipid B may be synthesized according to WO2014/136086 (e.g., pp. 107-09).

In some embodiments, the CCD lipid is Lipid C, which is 2-((4-(((3-(dimethylamino)propoxy)carbonyl)oxy)hexadecanoyl)oxy)propane-1,3-diyl (9Z,9′Z,12Z,12′Z)-bis(octadeca-9,12-dienoate). Lipid C can be depicted as:

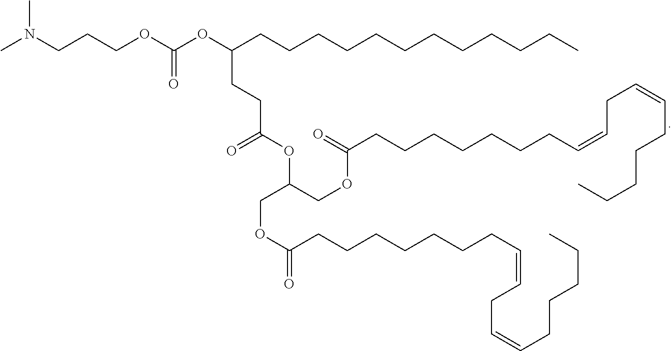 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

In some embodiments, the CCD lipid is Lipid D, which is 3-(((3-(dimethylamino)propoxy)carbonyl)oxy)-13-(octanoyloxy)tridecyl 3-octylundecanoate.

Lipid D can be depicted as:

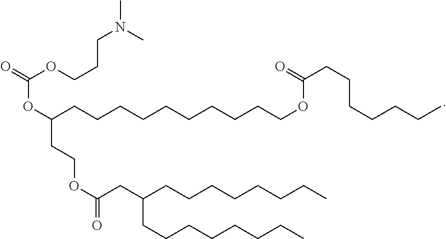 [SMILES: CCCCCCCCC(CCCCCCCC)CC(=O)OCCC(CCCCCCCCCCOC(=O)CCCCCCC)OC(=O)OCCCN(C)C]

Lipid C and Lipid D may be synthesized according to WO2015/095340.

The CCD lipid can also be an equivalent to Lipid A, Lipid B, Lipid C, or Lipid D. In certain embodiments, the CCD lipid is an equivalent to Lipid A, an equivalent to Lipid B, an equivalent to Lipid C, or an equivalent to Lipid D.

Amine Lipids

In some embodiments, the LNP compositions for the delivery of biologically active agents comprise an “amine lipid”, which is defined as Lipid A, Lipid B, Lipid C, Lipid D or equivalents of Lipid A (including acetal analogs of Lipid A), equivalents of Lipid B, equivalents of Lipid C, and equivalents of Lipid D.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

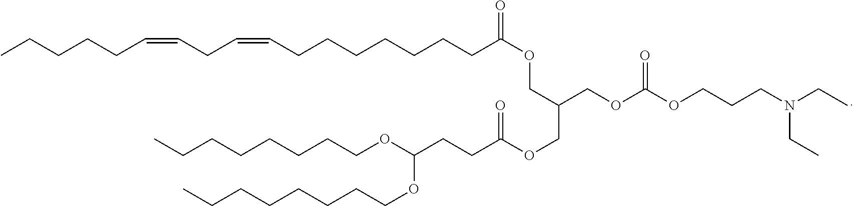 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In certain embodiments, the amine lipid is an equivalent to Lipid A.

In certain embodiments, an amine lipid is an analog of Lipid A. In certain embodiments, a Lipid A analog is an acetal analog of Lipid A. In particular LNP compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids suitable for use in the LNPs described herein are biodegradable in vivo and suitable for delivering a biologically active agent, such as an RNA to a cell. The amine lipids have low toxicity (e.g., are tolerated in an animal model without adverse effect in amounts of greater than or equal to 10 mg/kg of RNA cargo). In certain embodiments, LNPs comprising an amine lipid include those where at least 75% of the amine lipid is cleared from the plasma within 8, 10, 12, 24, or 48 hours, or 3, 4, 5, 6, 7, or 10 days. In certain embodiments, LNPs comprising an amine lipid include those where at least 50% of the mRNA or gRNA is cleared from the plasma within 8, 10, 12, 24, or 48 hours, or 3, 4, 5, 6, 7, or 10 days. In certain embodiments, LNPs comprising an amine lipid include those where at least 50% of the LNP is cleared from the plasma within 8, 10, 12, 24, or 48 hours, or 3, 4, 5, 6, 7, or 10 days, for example by measuring a lipid (e.g., an amine lipid), RNA (e.g., mRNA), or another component. In certain embodiments, lipid-encapsulated versus free lipid, RNA, or nucleic acid component of the LNP is measured.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of LNP compositions of the present disclosure.

The amine lipids may lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which an mRNA or a gRNA is cleared from the blood, serum, or plasma. In some embodiments, the clearance rate is the rate at which LNP is cleared from the blood, serum, or plasma. In some embodiments, the clearance rate is the rate at which LNP is cleared from a tissue, such as liver tissue or spleen tissue. In certain embodiments, a high clearance rate leads to a safety profile with no substantial adverse effects. The amine lipids may reduce LNP accumulation in circulation and in tissues. In some embodiments, a reduction in LNP accumulation in circulation and in tissues leads to a safety profile with no substantial adverse effects.

The amine lipids of the present disclosure are ionizable (e.g., may form a salt) depending upon the pH of the medium they are in. For example, in a slightly acidic medium, the amine lipids may be protonated and thus bear a positive charge. Conversely, in a slightly basic medium, such as, for example, blood, where pH is approximately 7.35, the amine lipids may not be protonated and thus bear no charge. In some embodiments, the amine lipids of the present disclosure may be protonated at a pH of at least about 9. In some embodiments, the amine lipids of the present disclosure may be protonated at a pH of at least about 9. In some embodiments, the amine lipids of the present disclosure may be protonated at a pH of at least about 10.

The pH at which an amine lipid is predominantly protonated is related to its intrinsic pKa. In some embodiments, the amine lipids of the present disclosure may each, independently, have a pKa in the range of from about 5.1 to about 7.4. In some embodiments, the amine lipids of the present disclosure may each, independently, have a pKa in the range of from about 5.5 to about 6.6. In some embodiments, the amine lipids of the present disclosure may each, independently, have a pKa in the range of from about 5.6 to about 6.4. In some embodiments, the amine lipids of the present disclosure may each, independently, have a pKa in the range of from about 5.8 to about 6.2. For example, the amine lipids of the present disclosure may each, independently, have a pKa in the range of from about 5.8 to about 6.5. The pKa of an amine lipid can be an important consideration in formulating LNPs as it has been found that cationic lipids with a pKa ranging from about 5.1 to about 7.4 are effective for delivery of cargo in vivo, e.g., to the liver. Furthermore, it has been found that cationic lipids with a pKa ranging from about 5.3 to about 6.4 are effective for delivery in vivo, e.g., to tumors. See, e.g., WO 2014/136086.

Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC). In another embodiment, the neutral phospholipid may be dipalmitoylphosphatidylcholine (DPPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid. PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chail length comprises about C10 to C20. The dialkylglycerol or dialkylglycamide group can further comprise one or more substituted alkyl groups. The chain lengths may be symmetrical or assymetric.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In certain embodiments, the PEG (e.g., conjugated to a lipid moiety or lipid, such as a stealth lipid), is a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits. However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound 5027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

LNP Formulations

The LNP may contain (i) an amine lipid for encapsulation and for endosomal escape, (ii) a neutral lipid for stabilization, (iii) a helper lipid, also for stabilization, and (iv) a stealth lipid, such as a PEG lipid. The neutral lipid may be omitted.

In some embodiments, an LNP composition may comprise an RNA component that includes one or more of an RNA-guided DNA-binding agent, a Cas nuclease mRNA, a Class 2 Cas nuclease mRNA, a Cas9 mRNA, and a gRNA. In some embodiments, an LNP composition includes an mRNA encoding a Class 2 Cas nuclease, e.g. S. pyogenes Cas9, and a gRNA as the RNA component. In certain embodiments, an LNP composition may comprise the RNA component, an amine lipid, a helper lipid, a neutral lipid, and a stealth lipid. In certain LNP compositions, the helper lipid is cholesterol. In other compositions, the neutral lipid is DSPC. In additional embodiments, the stealth lipid is PEG2k-DMG or PEG2k-C11. In certain embodiments, the LNP composition comprises Lipid A or an equivalent of Lipid A; a helper lipid; a neutral lipid; a stealth lipid; and a guide RNA. In certain compositions, the amine lipid is Lipid A. In certain compositions, the amine lipid is Lipid A or an acetal analog thereof; the helper lipid is cholesterol; the neutral lipid is DSPC; and the stealth lipid is PEG2k-DMG.

In certain embodiments, lipid compositions are described according to the respective molar ratios of the component lipids in the formulation. Embodiments of the present disclosure provide lipid compositions described according to the respective molar ratios of the component lipids in the formulation. In one embodiment, the mol-% of the amine lipid may be from about 30 mol-% to about 60 mol-%. In one embodiment, the mol-% of the amine lipid may be from about 40 mol-% to about 60 mol-%. In one embodiment, the mol-% of the amine lipid may be from about 45 mol-% to about 60 mol-%. In one embodiment, the mol-% of the amine lipid may be from about 50 mol-% to about 60 mol-%. In one embodiment, the mol-% of the amine lipid may be from about 55 mol-% to about 60 mol-%. In one embodiment, the mol-% of the amine lipid may be from about 50 mol-% to about 55 mol-%. In one embodiment, the mol-% of the amine lipid may be about 50 mol-%. In one embodiment, the mol-% of the amine lipid may be about 55 mol-%. In some embodiments, the amine lipid mol-% of the LNP batch will be ±30%, ±25%, ±20%, ±15%, ±10%, ±5%, or ±2.5% of the target mol-%. In some embodiments, the amine lipid mol-% of the LNP batch will be ±4 mol-%, ±3 mol-%, ±2 mol-%, ±1.5 mol-%, ±1 mol-%, ±0.5 mol-%, or ±0.25 mol-% of the target mol-%. All mol-% numbers are given as a fraction of the lipid component of the LNP compositions. In certain embodiments, LNP inter-lot variability of the amine lipid mol-% will be less than 15%, less than 10% or less than 5%.

In one embodiment, the mol-% of the neutral lipid, e.g., neutral phospholipid, may be from about 5 mol-% to about 15 mol-%. In one embodiment, the mol-% of the neutral lipid, e.g., neutral phospholipid, may be from about 7 mol-% to about 12 mol-%. In one embodiment, the mol-% of the neutral lipid, e.g., neutral phospholipid, may be from about 0 mol-% to about 5 mol-%. In one embodiment, the mol-% of the neutral lipid, e.g., neutral phospholipid, may be from about 0 mol-% to about 10 mol-%. In one embodiment, the mol-% of the neutral lipid, e.g., neutral phospholipid, may be from about 5 mol-% to about 10 mol-%. In one embodiment, the mol-% of the neutral lipid, e.g., neutral phospholipid, may be from about 8 mol-% to about 10 mol-%.

In one embodiment, the mol-% of the neutral lipid, e.g., neutral phospholipid, may be about 5 mol-%, about 6 mol-%, about 7 mol-%, about 8 mol-%, about 9 mol-%, about 10 mol-%, about 11 mol-%, about 12 mol-%, about 13 mol-%, about 14 mol-%, or about 15 mol-%. In one embodiment, the mol-% of the neutral lipid, e.g., neutral phospholipid, may be about 9 mol-%.

In one embodiment, the mol-% of the neutral lipid, e.g., neutral phospholipid, may be from about 1 mol-% to about 5 mol-%. In one embodiment, the mol-% of the neutral lipid may be from about 0.1 mol-% to about 1 mol-%. In one embodiment, the mol-% of the neutral lipid such as neutral phospholipid may be about 0.1 mol-%, about 0.2 mol-%, about 0.5 mol-%, 1 mol-%, about 1.5 mol-%, about 2 mol-%, about 2.5 mol-%, about 3 mol-%, about 3.5 mol-%, about 4 mol-%, about 4.5 mol-%, or about 5 mol-%.

In one embodiment, the mol-% of the neutral lipid, e.g., neutral phospholipid, may be less than about 1 mol-%. In one embodiment, the mol-% of the neutral lipid, e.g., neutral phospholipid, may be less than about 0.5 mol-%. In one embodiment, the mol-% of the neutral lipid, e.g., neutral phospholipid, may be about 0 mol-%, about 0.1 mol-%, about 0.2 mol-%, about 0.3 mol-%, about 0.4 mol-%, about 0.5 mol-%, about 0.6 mol-%, about 0.7 mol-%, about 0.8 mol-%, about 0.9 mol-%, or about 1 mol-%. In some embodiments, the formulations disclosed herein are free of neutral lipid (i.e., 0 mol-% neutral lipid). In some embodiments, the formulations disclosed herein are essentially free of neutral lipid (i.e., about 0 mol-% neutral lipid). In some embodiments, the formulations disclosed herein are free of neutral phospholipid (i.e., 0 mol-% neutral phospholipid). In some embodiments, the formulations disclosed herein are essentially free of neutral phospholipid (i.e., about 0 mol-% neutral phospholipid).

In some embodiments, the neutral lipid mol-% of the LNP batch will be ±30%, ±25%, ±20%, ±15%, ±10%, ±5%, or ±2.5% of the target neutral lipid mol-%. In certain embodiments, LNP inter-lot variability will be less than 15%, less than 10% or less than 5%.

In one embodiment, the mol-% of the helper lipid may be from about 20 mol-% to about 60 mol-%. In one embodiment, the mol-% of the helper lipid may be from about 25 mol-% to about 55 mol-%. In one embodiment, the mol-% of the helper lipid may be from about 25 mol-% to about 50 mol-%. In one embodiment, the mol-% of the helper lipid may be from about 25 mol-% to about 40 mol-%. In one embodiment, the mol-% of the helper lipid may be from about 30 mol-% to about 50 mol-%. In one embodiment, the mol-% of the helper lipid may be from about 30 mol-% to about 40 mol-%. In one embodiment, the mol-% of the helper lipid is adjusted based on amine lipid, neutral lipid, and PEG lipid concentrations to bring the lipid component to 100 mol-%. In one embodiment, the mol-% of the helper lipid is adjusted based on amine lipid and PEG lipid concentrations to bring the lipid component to 100 mol-%. In one embodiment, the mol-% of the helper lipid is adjusted based on amine lipid and PEG lipid concentrations to bring the lipid component to at least 99 mol-%. In some embodiments, the helper mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

In one embodiment, the mol-% of the PEG lipid may be from about 1 mol-% to about 10 mol-%. In one embodiment, the mol-% of the PEG lipid may be from about 2 mol-% to about 10 mol-%. In one embodiment, the mol-% of the PEG lipid may be from about 2 mol-% to about 8 mol-%. In one embodiment, the mol-% of the PEG lipid may be from about 2 mol-% to about 4 mol-%. In one embodiment, the mol-% of the PEG lipid may be from about 2.5 mol-% to about 4 mol-%. In one embodiment, the mol-% of the PEG lipid may be about 3 mol-%. In one embodiment, the mol-% of the PEG lipid may be about 2.5 mol-%. In some embodiments, the PEG lipid mol-% of the LNP batch will be ±30%, ±25%, ±20%, ±15%, ±10%, ±5%, or ±2.5% of the target PEG lipid mol-%. In certain embodiments, LNP inter-lot variability will be less than 15%, less than 10% or less than 5%.

In certain embodiments, the cargo includes a nucleic acid encoding an RNA-guided DNA-binding agent (e.g. a Cas nuclease, a Class 2 Cas nuclease, or Cas9), and a gRNA or a nucleic acid encoding a gRNA, or a combination of mRNA and gRNA. In one embodiment, an LNP composition may comprise a Lipid A or its equivalents. In some aspects, the amine lipid is Lipid A. In some aspects, the amine lipid is a Lipid A equivalent, e.g. an analog of Lipid A. In certain aspects, the amine lipid is an acetal analog of Lipid A. In various embodiments, an LNP composition comprises an amine lipid, a neutral lipid, a helper lipid, and a PEG lipid. In certain embodiments, the helper lipid is cholesterol. In certain embodiments, the neutral lipid is DSPC. In specific embodiments, PEG lipid is PEG2k-DMG. In some embodiments, an LNP composition may comprise a Lipid A, a helper lipid, a neutral lipid, and a PEG lipid. In some embodiments, an LNP composition comprises an amine lipid, DSPC, cholesterol, and a PEG lipid. In some embodiments, the LNP composition comprises a PEG lipid comprising DMG. In certain embodiments, the amine lipid is selected from Lipid A, and an equivalent of Lipid A, including an acetal analog of Lipid A. In additional embodiments, an LNP composition comprises Lipid A, cholesterol, DSPC, and PEG2k-DMG.

In various embodiments, an LNP composition comprises an amine lipid, a helper lipid, a neutral lipid, and a PEG lipid. In various embodiments, an LNP composition comprises an amine lipid, a helper lipid, a neutral phospholipid, and a PEG lipid. In various embodiments, an LNP composition comprises a lipid component that consists of an amine lipid, a helper lipid, a neutral lipid, and a PEG lipid. In various embodiments, an LNP composition comprises an amine lipid, a helper lipid, and a PEG lipid. In certain embodiments, an LNP composition does not comprise a neutral lipid, such as a neutral phospholipid. In various embodiments, an LNP composition comprises a lipid component that consists of an amine lipid, a helper lipid, and a PEG lipid. In certain embodiments, the neutral lipid is chosen from one or more of DSPC, DPPC, DAPC, DMPC, DOPC, DOPE, and DSPE. In certain embodiments, the neutral lipid is DSPC. In certain embodiments, the neutral lipid is DPPC. In certain embodiments, the neutral lipid is DAPC. In certain embodiments, the neutral lipid is DMPC. In certain embodiments, the neutral lipid is DOPC. In certain embodiments, the neutral lipid is DOPE. In certain embodiments, the neutral lipid is DSPE. In certain embodiments, the helper lipid is cholesterol. In specific embodiments, the PEG lipid is PEG2k-DMG. In some embodiments, an LNP composition may comprise a Lipid A, a helper lipid, and a PEG lipid. In some embodiments, an LNP composition may comprise a lipid component that consists of Lipid A, a helper lipid, and a PEG lipid. In some embodiments, an LNP composition comprises an amine lipid, cholesterol, and a PEG lipid. In some embodiments, an LNP composition comprises a lipid component that consists of an amine lipid, cholesterol, and a PEG lipid. In some embodiments, the LNP composition comprises a PEG lipid comprising DMG. In certain embodiments, the amine lipid is selected from Lipid A and an equivalent of Lipid A, including an acetal analog of Lipid A. In certain embodiments, the amine lipid is a C5-C12 or a C4-C12 acetal analog of Lipid A. In additional embodiments, an LNP composition comprises Lipid A, cholesterol, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, an LNP composition may comprise a lipid component that comprises an amine lipid, a helper lipid, a neutral lipid, and a PEG lipid; and a nucleic acid component, wherein the N/P ratio is about 3 to 10. In some embodiments, an LNP composition may comprise a lipid component that comprises an amine lipid, a helper lipid, and a PEG lipid; and a nucleic acid component, wherein the N/P ratio is about 3 to 10. In some embodiments, an LNP composition may comprise a lipid component that comprises an amine lipid, a helper lipid, a neutral lipid, and a helper lipid; and an RNA component, wherein the N/P ratio is about 3 to 10. In some embodiments, an LNP composition may comprise a lipid component that comprises an amine lipid, a helper lipid, and a PEG lipid; and an RNA component, wherein the N/P ratio is about 3 to 10. In one embodiment, the N/P ratio may be about 5 to 7. In one embodiment, the N/P ration may be about 3 to 7. In one embodiment, the N/P ratio may be about 4.5 to 8. In one embodiment, the N/P ratio may be about 6. In one embodiment, the N/P ratio may be 6±1. In one embodiment, the N/P ratio may be 6±0.5. In some embodiments, the N/P ratio will be ±30%, ±25%, ±20%, ±15%, ±10%, ±5%, or ±2.5% of the target N/P ratio. In certain embodiments, LNP inter-lot variability will be less than 15%, less than 10% or less than 5%.

In some embodiments, the RNA component may comprise a nucleic acid, such as a nucleic acid disclosed herein, e.g., encoding a Cas nuclease. In one embodiment, RNA component may comprise a Cas9 mRNA. In some compositions comprising a nucleic acid encoding a Cas nuclease, the LNP further comprises a gRNA nucleic acid, such as a gRNA. In some embodiments, the RNA component comprises a Cas nuclease mRNA and a gRNA. In some embodiments, the RNA component comprises a Class 2 Cas nuclease mRNA and a gRNA. In any of the foregoing embodiments, the gRNA may be an sgRNA described herein, such as a chemically modified sgRNA described herein.

In certain embodiments, an LNP composition may comprise a nucleic acid disclosed herein, e.g., encoding a Cas nuclease, such as a Class 2 Cas nuclease, a gRNA, an amine lipid, a helper lipid, a neutral lipid, and a PEG lipid. In certain LNP compositions, the helper lipid is cholesterol; the neutral lipid is DSPC; and/or the PEG lipid is PEG2k-DMG or PEG2k-C11. In specific compositions, the amine lipid is selected from Lipid A and its equivalents, such as an acetal analog of Lipid A. In one embodiment, the lipid component of the LNP composition consists of an amine lipid, a helper lipid, a neutral lipid, and a PEG lipid. In one embodiment, the lipid component of the LNP composition consists of an amine lipid, a helper lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. Certain compositions comprising an mRNA encoding a Cas nuclease and a gRNA comprise less than about 1 mol-% neutral lipid, e.g. neutral phospholipid. Certain compositions comprising an mRNA encoding a Cas nuclease and a gRNA comprise less than about 0.5 mol-% neutral lipid, e.g. neutral phospholipid. In certain compositions, the LNP does not comprise a neutral lipid, e.g., neutral phospholipid. In additional embodiments comprising an mRNA encoding a Cas nuclease and a gRNA, the PEG lipid is PEG2k-DMG or PEG2k-C11. In certain embodiments, the amine lipid is selected from Lipid A and its equivalents, such as acetal analogs of Lipid A.

In one embodiment, an LNP composition may comprise an sgRNA. In one embodiment, an LNP composition may comprise a Cas9 sgRNA. In one embodiment, an LNP composition may comprise a Cpf1 sgRNA. In some compositions comprising an sgRNA, the LNP includes an amine lipid, a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In certain embodiments, the amine lipid is selected from Lipid A and its equivalents, such as acetal analogs of Lipid A.

In certain embodiments, the LNP compositions include a Cas nuclease mRNA, such as a Class 2 Cas mRNA and at least one gRNA. In certain embodiments, the LNP composition includes a ratio of gRNA to Cas nuclease mRNA, such as Class 2 Cas nuclease mRNA from about 25:1 to about 1:25. In certain embodiments, the LNP formulation includes a ratio of gRNA to Cas nuclease mRNA, such as Class 2 Cas nuclease mRNA from about 10:1 to about 1:10. In certain embodiments, the LNP formulation includes a ratio of gRNA to Cas nuclease mRNA, such as Class 2 Cas nuclease mRNA from about 8:1 to about 1:8. As measured herein, the ratios are by weight. In some embodiments, the LNP formulation includes a ratio of gRNA to Cas nuclease mRNA, such as Class 2 Cas mRNA from about 5:1 to about 1:5. In some embodiments, ratio range is about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. In some embodiments, the gRNA to mRNA ratio is about 3:1 or about 2:1 In some embodiments the ratio of gRNA to Cas nuclease mRNA, such as Class 2 Cas nuclease is about 1:1. The ratio may be about 25:1, 10:1, 5:1, 3:1, 1:1, 1:3, 1:5, 1:10, or 1:25.

In some embodiments, LNPs are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of LNPs, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, tangential flow filtration, or chromatography. The LNPs may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension.

Moreover, the LNP compositions are biodegradable, in that they do not accumulate to cytotoxic levels in vivo at a therapeutically effective dose. In some embodiments, the LNP compositions do not cause an innate immune response that leads to substantial adverse effects at a therapeutic dose level. In some embodiments, the LNP compositions provided herein do not cause toxicity at a therapeutic dose level.

In some embodiments, the pdi may range from about 0.005 to about 0.75. In some embodiments, the pdi may range from about 0.01 to about 0.5. In some embodiments, the pdi may range from about zero to about 0.4. In some embodiments, the pdi may range from about zero to about 0.35. In some embodiments, the pdi may range from about zero to about 0.35. In some embodiments, the pdi may range from about zero to about 0.3. In some embodiments, the pdi may range from about zero to about 0.25. In some embodiments, the pdi may range from about zero to about 0.2. In some embodiments, the pdi may be less than about 0.08, 0.1, 0.15, 0.2, or 0.4.

The LNPs disclosed herein have a size (e.g., Z-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcps. The data is presented as a weighted-average of the intensity measure (Z-average diameter).

In some embodiments, the LNPs are formed with an average encapsulation efficiency ranging from about 50% to about 100%. In some embodiments, the LNPs are formed with an average encapsulation efficiency ranging from about 50% to about 70%. In some embodiments, the LNPs are formed with an average encapsulation efficiency ranging from about 70% to about 90%. In some embodiments, the LNPs are formed with an average encapsulation efficiency ranging from about 90% to about 100%. In some embodiments, the LNPs are formed with an average encapsulation efficiency ranging from about 75% to about 95%.

In some embodiments, the LNPs are formed with an average molecular weight ranging from about 1.00E+05 g/mol to about 1.00E+10 g/mol. In some embodiments, the LNPs are formed with an average molecular weight ranging from about 5.00E+05 g/mol to about 7.00E+07 g/mol. In some embodiments, the LNPs are formed with an average molecular weight ranging from about 1.00E+06 g/mol to about 1.00E+10 g/mol. In some embodiments, the LNPs are formed with an average molecular weight ranging from about 1.00E+07 g/mol to about 1.00E+09 g/mol. In some embodiments, the LNPs are formed with an average molecular weight ranging from about 5.00E+06 g/mol to about 5.00E+09 g/mol.

In some embodiments, the polydispersity (Mw/Mn; the ratio of the weight averaged molar mass (Mw) to the number averaged molar mass (Mn)) may range from about 1.000 to about 2.000. In some embodiments, the Mw/Mn may range from about 1.00 to about 1.500. In some embodiments, the Mw/Mn may range from about 1.020 to about 1.400. In some embodiments, the Mw/Mn may range from about 1.010 to about 1.100. In some embodiments, the Mw/Mn may range from about 1.100 to about 1.350.

Dynamic Light Scattering (“DLS”) can be used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero. In some embodiments, the pdi may range from 0.005 to 0.75. In some embodiments, the pdi may range from 0.01 to 0.5. In some embodiments, the pdi may range from 0.02 to 0.4. In some embodiments, the pdi may range from 0.03 to 0.35. In some embodiments, the pdi may range from 0.1 to 0.35.

In some embodiments, LNPs disclosed herein have a size of 1 to 250 nm. In some embodiments, the LNPs have a size of 10 to 200 nm. In further embodiments, the LNPs have a size of 20 to 150 nm. In some embodiments, the LNPs have a size of 50 to 150 nm. In some embodiments, the LNPs have a size of 50 to 100 nm. In some embodiments, the LNPs have a size of 50 to 120 nm. In some embodiments, the LNPs have a size of 75 to 150 nm. In some embodiments, the LNPs have a size of 30 to 200 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcts. The data is presented as a weighted-average of the intensity measure. In some embodiments, the LNPs are formed with an average encapsulation efficiency ranging from 50% to 100%. In some embodiments, the LNPs are formed with an average encapsulation efficiency ranging from 50% to 70%. In some embodiments, the LNPs are formed with an average encapsulation efficiency ranging from 70% to 90%. In some embodiments, the LNPs are formed with an average encapsulation efficiency ranging from 90% to 100%. In some embodiments, the LNPs are formed with an average encapsulation efficiency ranging from 75% to 95%.

In some embodiments, LNPs associated with the gRNAs disclosed herein are for use in preparing a medicament for treating ATTR. In some embodiments, LNPs associated with the gRNAs disclosed herein are for use in preparing a medicament for reducing or preventing accumulation and aggregation of TTR in amyloids or amyloid fibrils in subjects having ATTR. In some embodiments, LNPs associated with the gRNAs disclosed herein are for use in preparing a medicament for reducing serum TTR concentration. In some embodiments, LNPs associated with the gRNAs disclosed herein are for use in treating ATTR in a subject, such as a mammal, e.g., a primate such as a human. In some embodiments, LNPs associated with the gRNAs disclosed herein are for use in reducing or preventing accumulation and aggregation of TTR in amyloids or amyloid fibrils in subjects having ATTR, such as a mammal, e.g., a primate such as a human. In some embodiments, LNPs associated with the gRNAs disclosed herein are for use in reducing serum TTR concentration in a subject, such as a mammal, e.g., a primate such as a human. In any of the foregoing embodiments, the LNPs may be associated with the gRNAs disclosed herein and nucleic acids (e.g., mRNA) encoding an RNA-guided DNA binding agent (e.g. Cas9, Spy Cas9) disclosed herein.

Electroporation is also a well-known means for delivery of cargo, and any electroporation methodology may be used for delivery of any one of the gRNAs disclosed herein. In some embodiments, electroporation may be used to deliver any one of the gRNAs disclosed herein, and optionally an RNA-guided DNA nuclease such as Cas9 or a nucleic acid encoding an RNA-guided DNA nuclease such as Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also optionally associated with an RNA-guided DNA nuclease such as Cas9 or a nucleic acid encoding an RNA-guided DNA nuclease, e.g., a nucleic acid (e.g., mRNA) encoding an RNA-guided DNA binding agent (e.g. Cas9, Spy Cas9) disclosed herein.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and optionally nucleic acids described herein encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA, and optionally a nucleic acid described herein encoding an RNA-guided DNA nuclease, which can be a Cas nuclease, such as Cas9 or Cpf1. In some embodiments, the vector comprises one or more nucleotide sequence(s) encoding a crRNA, a trRNA, and optionally a nucleic acid described herein encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In some embodiments, the viral vector is AAV2, AAV3, AAV3B, AAVS, AAV6, AAV6.2, AAV7, AAVrh.64R1, AAVhu.37, AAVrh.8, AAVrh.32.33, AAV8, AAV9, AAVrh10, or AAVLK03. In other embodiments, the viral vector may a lentivirus vector.

In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding an RNA-guided DNA nuclease such as a Cas nuclease, while a second AAV vector may contain one or more guide sequences.

In some embodiments, the vector may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding the guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the vector may optionally further comprise a nucleotide sequence encoding an RNA-guided DNA nuclease such as a nuclease described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the nucleotide sequence encoding the guide RNA may be located on the same vector comprising the nucleotide sequence encoding an RNA-guided DNA nuclease such as a Cas nuclease. In some embodiments, expression of the guide RNA and of the RNA-guided DNA nuclease such as a Cas protein may be driven by their own corresponding promoters. In some embodiments, expression of the guide RNA may be driven by the same promoter that drives expression of the RNA-guided DNA nuclease such as a Cas protein. In some embodiments, the guide RNA and the RNA-guided DNA nuclease such as a Cas protein transcript may be contained within a single transcript. For example, the guide RNA may be within an untranslated region (UTR) of the RNA-guided DNA nuclease such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA nuclease such as a Cas protein and the guide RNA from the same vector in close temporal proximity may facilitate more efficient formation of the CRISPR RNP complex.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

The vector may be delivered by liposome, a nanoparticle, an exosome, or a microvesicle. The vector may also be delivered by a lipid nanoparticle (LNP); see e.g., WO2017/173054, published Oct. 5, 2017, entitled “LIPID NANOPARTICLE FORMULATIONS FOR CRISPR/CAS COMPONENTS,” and WO2019067992A1 published Apr. 4, 2019, entitled “FORMULATIONS,” the contents of each of which are hereby incorporated by reference in their entirety. Any of the LNPs and LNP formulations described herein are suitable for delivery of the guides alone or together a cas nuclease or a nucleic acid encoding a cas nuclease. In some embodiments, an LNP composition is encompassed comprising: an RNA component and a lipid component, wherein the lipid component comprises an amine lipid, a neutral lipid, a helper lipid, and a stealth lipid; and wherein the N/P ratio is about 1-10.

In some instances, the lipid component comprises Lipid A or its acetal analog, cholesterol, DSPC, and PEG-DMG; and wherein the N/P ratio is about 1-10. In some embodiments, the lipid component comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the lipid component comprises about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some instances, the lipid component comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is about 3-8. In some instances, the lipid component comprises: 48-53 mol-% Lipid A; about 8-10 mol-% DSPC; and 1.5-10 mol-% PEG lipid, wherein the remainder of the lipid component is cholesterol, and wherein the N/P ratio of the LNP composition is 3-8±0.2.

In some embodiments, the LNP comprises a lipid component and the lipid component comprises, consists essentially of, or consists of: about 50 mol-% amine lipid such as Lipid A; about 9 mol-% neutral lipid such as DSPC; about 3 mol-% of a stealth lipid such as a PEG lipid, such as PEG2k-DMG, and the remainder of the lipid component is helper lipid such as cholesterol, wherein the N/P ratio of the LNP composition is about 6. In some embodiments, the amine lipid is Lipid A. In some embodiments, the neutral lipid is DSPC. In some embodiments, the stealth lipid is a PEG lipid. In some embodiments, the stealth lipid is a PEG2k-DMG. In some embodiments, the helper lipid is cholesterol. In some embodiments, the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

In some embodiments, the vector may be delivered systemically. In some embodiments, the vector may be delivered into the hepatic circulation.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated Streptococcus pyogenes (“Spy”) Cas9 mRNA containing N1-methyl pseudo-U was generated by in vitro transcription using a linearized plasmid DNA template and T7 RNA polymerase. Plasmid DNA containing a T7 promoter, a sequence for transcription according to SEQ ID NO: 1, 2, or another sequence disclosed herein, and a 90-100 nt poly (A/T) region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate Cas9 modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The Cas9 mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

When SEQ ID NOs: 1 and 2 are referred to below with respect to RNAs, it is understood that Ts should be replaced with Us (which were N1-methyl pseudouridines as described above). Cas9 mRNAs used in the Examples include a 5′ cap and a 3′ poly-A tail, e.g., up to 100 nts, and are identified by SEQ ID NO.

Human TTR Guide Design and Human TTR with Cynomolgus Monkey Homology Guide Design

Initial guide selection was performed in silico using a human reference genome (e.g., hg38) and user defined genomic regions of interest (e.g., TTR protein coding exons), for identifying PAMs in the regions of interest. For each identified PAM, analyses were performed and statistics reported. gRNA molecules were further selected and rank ordered based on a number of criteria (e.g., GC content, predicted on-target activity, and potential off-target activity).

A total of 68 guide RNAs were designed toward TTR (ENSG00000118271) targeting the protein coding regions within Exon 1, 2, 3 and 4. Of the total 68 guides, 33 were 100% homologous in cynomolgus monkey (“cyno”). In addition, for 10 of the human TTR guides which were not perfectly homologous in cyno, “surrogate” guides were designed and made in parallel to perfectly match the corresponding cyno target sequence. These “surrogate” or “tool” guides may be screened in cyno, e.g., to approximate the activity and function of the homologous human guide sequence. Guide sequences and corresponding genomic coordinates are provided (Table 1). All of the guide RNAs were made as dual guide RNAs, and a subset of the guide sequences were made as modified single guide RNA (Table 2). Guide ID alignment across dual guide RNA (dgRNA) IDs, modified single guide RNA (sgRNA) IDs, the number of mismatches to the cyno genome as well as the cyno exact matched IDs are provided (Table 3). Where dgRNAs are used in the experiments detailed throughout the Examples, SEQ ID NO: 270 was used.

The sgRNAs in the following examples were chemically synthesized by known methods using phosphoramidites.

Cas9 mRNA and Guide RNA Delivery In Vitro

HEK293_Cas9 cell line. The human embryonic kidney adenocarcinoma cell line HEK293 constitutively expressing Spy Cas9 (“HEK293_Cas9”) was cultured in DMEM media supplemented with 10% fetal bovine serum and 500 μg/ml G418. Cells were plated at a density of 10,000 cells/well in a 96-well plate 24 hours prior to transfection. Cells were transfected with Lipofectamine RNAiMAX (ThermoFisher, Cat. 13778150) per the manufacturer's protocol. Cells were transfected with a lipoplex containing individual crRNA (25 nM), trRNA (25 nM), Lipofectamine RNAiMAX (0.3 μL/well) and OptiMem.

HUH7 cell line. The human hepatocellular carcinoma cell line HUH7 (Japanese Collection of Research Bioresources Cell Bank, Cat. JCRB0403) was cultured in DMEM media supplemented with 10% fetal bovine serum. Cells were plated on at a density of 15,000 cells/well in a 96-well plate 20 hours prior to transfection. Cells were transfected with Lipofectamine MessengerMAX (ThermoFisher, Cat. LMRNA003) per the manufacturer's protocol. Cells were sequentially transfected with a lipoplex containing Spy Cas9 mRNA (100 ng), MessengerMAX (0.3 μL/well) and OptiMem followed by a separate lipoplex containing individual crRNA (25 nM), tracer RNA (25 nM), MessengerMAX (0.3 μL/well) and OptiMem.

HepG2 cell line. The human hepatocellular carcinoma cell line HepG2 (American Type Culture Collection, Cat. HB-8065) was cultured in DMEM media supplemented with 10% fetal bovine serum. Cells were counted and plated on Bio-coat collagen I coated 96-well plates (ThermoFisher, Cat. 877272) at a density of 10,000 cells/well in a 96-well plate 24 hours prior to transfection. Cells were transfected with Lipofectamine 2000 (ThermoFisher, Cat. 11668019) per the manufacturer's protocol. Cells were sequentially transfected with lipoplex containing Spy Cas9 mRNA (100 ng), Lipofectamine 2000 (0.2 μL/well) and OptiMem followed by a separate lipoplex containing individual crRNA (25 nM), tracer RNA (25 nM), Lipofectamine 2000 (0.2 μL/well) and OptiMem.

Primary liver hepatocytes. Primary human liver hepatocytes (PHH) and primary cynomolgus liver hepatocytes (PCH) (Gibco) were cultured per the manufacturer's protocol (Invitrogen, protocol 11.28.2012). In brief, the cells were thawed and resuspended in hepatocyte thawing medium with supplements (Gibco, Cat. CM7000) followed by centrifugation at 100 g for 10 minutes for human and 80 g for 4 minutes for cyno. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (ThermoFisher, Cat. 877272) at a density of 33,000 cells/well for human or 60,000 cells/well for cyno (or 65,000 cells/well when assaying effects on TTR protein, described further below). Plated cells were allowed to settle and adhere for 6 or 24 hours in a tissue culture incubator at 37° C. and 5% CO₂ atmosphere. After incubation cells were checked for monolayer formation and media was replaced with hepatocyte culture medium with serum-free supplement pack (Invitrogen, Cat. A1217601 and CM4000).

Lipofectamine RNAiMax (ThermoFisher, Cat. 13778150) based transfections were conducted as per the manufacturer's protocol. Cells were sequentially transfected with a lipoplex containing Spy Cas9 mRNA (100 ng), Lipofectamine RNAiMax (0.4 μL/well) and OptiMem followed by a separate lipoplex containing crRNA (25 nM) and tracer RNA (25 nM) or sgRNA (25 nM), Lipofectamine RNAiMax (0.4 μL/well) and OptiMem.

Ribonucleotide formation was performed prior to electroporation or transfection of Spy Cas9 protein loaded with guide RNAs (RNPs) onto cells. For dual guide (dgRNAs), individual crRNA and trRNA was pre-annealed by mixing equivalent amounts of reagent and incubating at 95° C. for 2 min and cooling to room temperature. Single guide (sgRNAs) were boiled at 95° C. for 2 min and cooling to room temperature. The boiled dgRNA or sgRNA was incubated with Spy Cas9 protein in Optimem for 10 minutes at room temperature to form a ribonucleoprotein (RNP) complex.

For RNP electroporation into primary human and cyno hepatocytes, the cells are thawed and resuspended in Lonza electroporation Primary Cell P3 buffer at a concentration of 2500 cells per μL for human hepatocytes and 3500 cells per μL for cyno hepatocytes. A volume of 20 μL of resuspended cells and 5 μL of RNP are mixed together per guide. 20 μL of the mixture is placed into a Lonza electroporation plate. The cells were electroporated using the Lonza nucleofector with the preset protocol EX-147. Post electroporation, the cells are transferred into a Biocoat plate containing pre-warmed maintenance media and placed in a tissue culture incubator at 37° C. and 5% CO₂.

For RNP lipoplex transfections, cells were transfected with Lipofectamine RNAiMAX (ThermoFisher, Cat. 13778150) per the manufacturer's protocol. Cells were transfected with an RNP containing Spy Cas9 (10 nM), individual guide (10 nM), tracer RNA (10 nM), Lipofectamine RNAiMAX (1.0 μL/well) and OptiMem. RNP formation was performed as described above.

LNPs were formed either by microfluidic mixing of the lipid and RNA solutions using a Precision Nanosystems NanoAssemblr™ Benchtop Instrument, per the manufacturer's protocol, or cross-flow mixing.

### LNP Formulation—NanoAssemblr

In general, the lipid nanoparticle components were dissolved in 100% ethanol with the lipid component of various molar ratios. The RNA cargos were dissolved in 25 mM citrate, 100 mM NaCl, pH 5.0, resulting in a concentration of RNA cargo of approximately 0.45 mg/mL. The LNPs were formulated with a lipid amine to RNA phosphate (N:P) molar ratio of about 4.5 or about 6, with the ratio of mRNA to gRNA at 1:1 by weight.

The LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates. After mixing, the LNPs were collected, diluted in water (approximately 1:1 v/v), held for 1 hour at room temperature, and further diluted with water (approximately 1:1 v/v) before final buffer exchange. The final buffer exchange into 50 mM Tris, 45 mM NaCl, 5% (w/v) sucrose, pH 7.5 (TSS) was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at −80° C. until further use.

### LNP Formulation—Cross-Flow

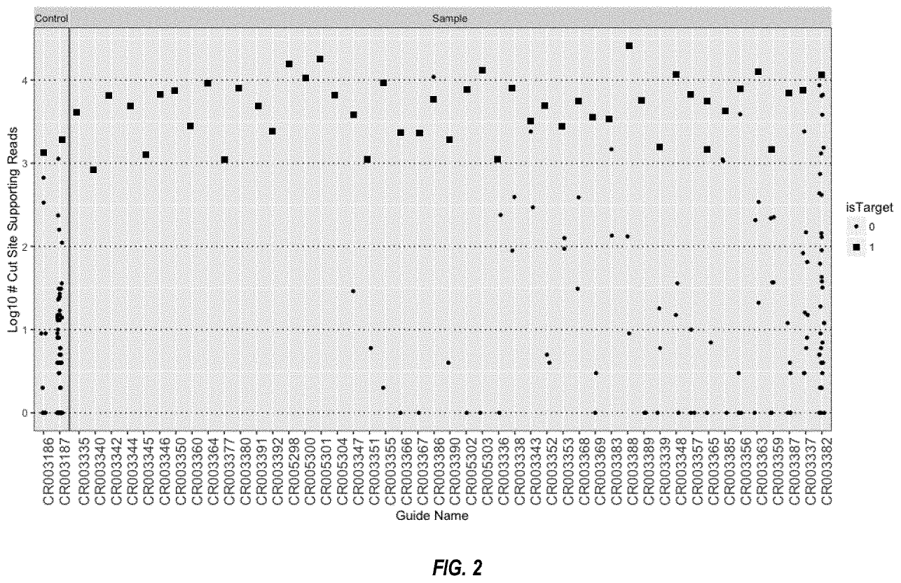

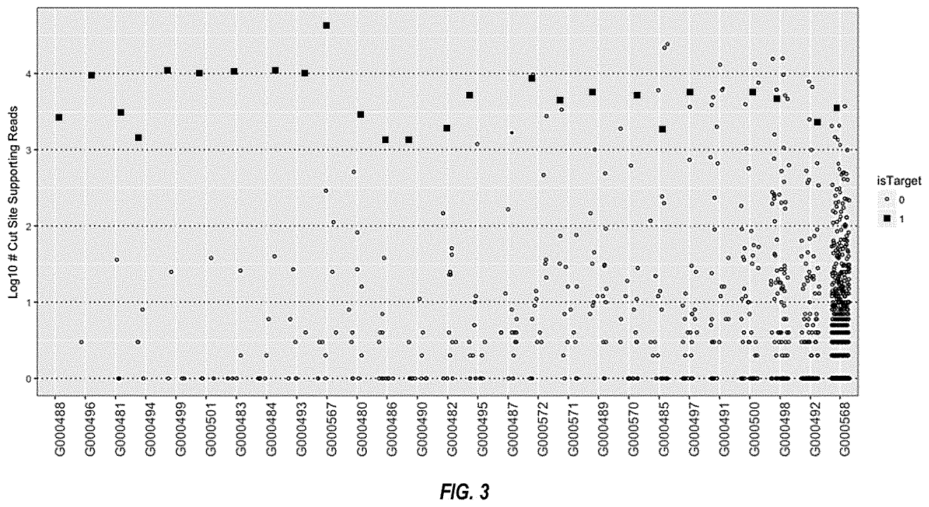

For LNPs prepared using the cross-flow technique, the LNPs were formed by impinging jet mixing of the lipid in ethanol with two volumes of RNA solutions and one volume of water. The lipid in ethanol is mixed through a mixing cross with the two volumes of RNA solution. A fourth stream of water is mixed with the outlet stream of the cross through an inline tee. (See WO2016010840 FIG. 2.) The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, formulations were concentrated by centrifugation with Amicon 100 kDa centrifugal filters (Millipore). The resulting mixture was then filtered using a 0.2 μm sterile filter. The final LNP was stored at 4° C. or −80° C. until further use.

### Formulation Analytics

Dynamic Light Scattering (“DLS”) is used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero. Average particle size and polydispersity are measured by dynamic light scattering (DLS) using a Malvern Zetasizer DLS instrument. LNP samples were diluted 30× in PBS prior to being measured by DLS. Z-average diameter which is an intensity based measurement of average particle size was reported along with number average diameter and pdi. A Malvern Zetasizer instrument is also used to measure the zeta potential of the LNP. Samples are diluted 1:17 (50 uL into 800 uL) in 0.1×PBS, pH 7.4 prior to measurement.

Electrophoretic light scattering is used to characterize the surface charge of the LNP at a specified pH. The surface charge, or the zeta potential, is a measure of the magnitude of electrostatic repulsion/attraction between particles in the LNP suspension.

Asymmetric-Flow Field Flow Fractionation—Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation).

Nanoparticle tracking analysis (NTA, Malvern Nanosight) can be used to determine particle size distribution as well as particle concentration. LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. The instrument also counts the number of individual particles counted in the analysis to give particle concentration.

Cryo-electron microscopy (“cryo-EM”) can be used to determine the particle size, morphology, and structural characteristics of an LNP.

Lipid compositional analysis of the LNPs can be determined from liquid chromatography followed by charged aerosol detection (LC-CAD). This analysis can provide a comparison of the actual lipid content versus the theoretical lipid content.

LNP compositions are analyzed for average particle size, polydispersity index (pdi), total RNA content, encapsulation efficiency of RNA, and zeta potential. LNP compositions may be further characterized by lipid analysis, AF4-MALS, NTA, and/or cryo-EM. Average particle size and polydispersity are measured by dynamic light scattering (DLS) using a Malvern Zetasizer DLS instrument. LNP samples were diluted with PBS buffer prior to being measured by DLS. Z-average diameter which is an intensity-based measurement of average particle size is reported along with number average diameter and pdi. A Malvern Zetasizer instrument is also used to measure the zeta potential of the LNP. Samples are diluted 1:17 (50 μL into 800 μL) in 0.1×PBS, pH 7.4 prior to measurement.

A fluorescence-based assay (Ribogreen®, ThermoFisher Scientific) is used to determine total RNA concentration and free RNA. LNP samples are diluted appropriately with 1×TE buffer containing 0.2% Triton-X 100 to determine total RNA or 1×TE buffer to determine free RNA. Standard curves are prepared by utilizing the starting RNA solution used to make the compositions and diluted in 1×TE buffer+/−0.2% Triton-X 100. Diluted RiboGreen® dye (according to the manufacturer's instructions) is then added to each of the standards and samples and allowed to incubate for approximately 10 minutes at room temperature, in the absence of light. A SpectraMax M5 Microplate Reader (Molecular Devices) is used to read the samples with excitation, auto cutoff and emission wavelengths set to 488 nm, 515 nm, and 525 nm respectively. Total RNA and free RNA are determined from the appropriate standard curves.

Encapsulation efficiency is calculated as (Total RNA−Free RNA)/Total RNA. The same procedure may be used for determining the encapsulation efficiency of a DNA-based cargo component. In a fluorescence-based assay, for single-strand DNA Oligreen Dye may be used, and for double-strand DNA, Picogreen Dye. Alternatively, the total RNA concentration can be determined by a reverse-phase ion-pairing (RP-IP) HPLC method. Triton X-100 is used to disrupt the LNPs, releasing the RNA. The RNA is then separated from the lipid components chromatographically by RP-IP HPLC and quantified against a standard curve using UV absorbance at 260 nm.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debeye model to determine molecular weight and rms radius from the detector signals.

Lipid components in LNPs are analyzed quantitatively by HPLC coupled to a charged aerosol detector (CAD). Chromatographic separation of 4 lipid components is achieved by reverse phase HPLC. CAD is a destructive mass-based detector which detects all non-volatile compounds and the signal is consistent regardless of analyte structure.

Typically, when preparing LNPs, encapsulation was >80%, particle size was <120 nm, and pdi was <0.2.

### LNP Delivery In Vivo

Unless otherwise noted, CD-1 female mice, ranging from 6-10 weeks of age were used in each study. Animals were weighed and grouped according to body weight for preparing dosing solutions based on group average weight. LNPs were dosed via the lateral tail vein in a volume of 0.2 mL per animal (approximately 10 mL per kilogram body weight). The animals were observed at approximately 6 hours post dose for adverse effects. Body weight was measured at twenty-four hours post-administration, and animals were euthanized at various time points by exsanguination via cardiac puncture under isoflourane anesthesia. Blood was collected into serum separator tubes or into tubes containing buffered sodium citrate for plasma as described herein. For studies involving in vivo editing, liver tissue was typically collected from the median lobe or from three independent lobes (e.g., the right median, left median, and left lateral lobes) from each animal for DNA extraction and analysis.

### Transthyretin (TTR) ELISA Analysis Used in Animal Studies

Blood was collected and the serum was isolated as indicated. The total mouse TTR serum levels were determined using a Mouse Prealbumin (Transthyretin) ELISA Kit (Aviva Systems Biology, Cat. OKIA00111); rat TTR serum levels were measured using a rat specific ELISA kit (Aviva Systems Biology catalog number OKIA00159); human TTR serum levels were measured using a human specific ELISA kit (Aviva Systems Biology catalog number OKIA00081); each according to manufacture's protocol. Briefly, sera were serial diluted with kit sample diluent to a final dilution of 10,000-fold, or 5,000-fold when measuring human TTR in mouse sera. 100 ul of the prepared standard curve or diluted serum samples were added to the ELISA plate, incubated for 30 minutes at room temperature then washed 3 times with provided wash buffer. 100 uL of detection antibody was then added to each well and incubated for 20 minutes at room temperature followed by 3 washes. 100 uL of substrate is added then incubated for 10 minutes at room temperature before the addition of 100 uL stop solution. The absorbance of the contents was measured on the Spectramax M5 plate reader with analysis using SoftmaxPro version 7.0 software. Serum TTR levels were quantitated off the standard curve using 4 parameter logistic fit and expressed as ug/mL of serum or percent knockdown relative control (vehicle treated) animals.

### Genomic DNA Isolation

Transfected cells were harvested post-transfection at 24, 48, or 72 hours. The genomic DNA was extracted from each well of a 96-well plate using 50 μL/well BuccalAmp DNA Extraction solution (Epicentre, Cat. QE09050) per manufacturer's protocol. All DNA samples were subjected to PCR and subsequent NGS analyses, as described herein.

### Next-Generation Sequencing (“NGS”) Analysis

To quantitatively determine the efficiency of editing at the target location in the genome, sequencing was utilized to identify the presence of insertions and deletions introduced by gene editing.

Primers were designed around the target site within the gene of interest (e.g. TTR), and the genomic area of interest was amplified.

Additional PCR was performed per the manufacturer's protocols (Illumina) to add chemistry for sequencing. The amplicons were sequenced on an Illumina MiSeq instrument. The reads were aligned to a reference genome (e.g., the human reference genome (hg38), the cynomologus reference genome (mf5), the rat reference genome (rn6), or the mouse reference genome (mm10)) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing” or “indel frequency”) is defined as the total number of sequence reads with insertions/deletions (“indels”) or substitutions over the total number of sequence reads, including wild type.

### Analysis of Secreted Transthyretin (“TTR”) Protein by Western Blot

Secreted levels of TTR protein in media were determined using western blotting methods. HepG2 cells were transfected as previously described with select guides from Table 1. Media changes were performed every 3 days post transfection. Six days post-transfection, the media was removed, and cells were washed once with media that did not contain fetal bovine serum (FBS). Media without serum was added to the cells and incubated at 37° C. After 4 hrs the media was removed and centrifuged to pellet any debris; cell number for each well was estimated based on the values obtained from a CTG assay on remaining cells and comparison to the plate average. After centrifugation, the media was transferred to a new plate and stored at −20° C. An acetone precipitation of the media was performed to precipitate any protein that had been secreted into the media. Four volumes of ice cold acetone were added to one volume of media. The solution was mixed well and kept at −20° C. for 90 min. The acetone:media mixture was centrifuged at 15,000×g and 4° C. for 15 min. The supernatant was discarded and the retained pellet was air dried to eliminate any residual acetone. The pellet was resuspended in 154 RIPA buffer (Boston Bio Products, Cat. BP-115) plus freshly added protease inhibitor mixture consisting of complete protease inhibitor cocktail (Sigma, Cat. 11697498001) and 1 mM DTT. Lysates were mixed with Laemmli buffer and denatured at 95° C. for 10 minutes. Western blots were run using the NuPage system on 12% Bis-Tris gels (ThermoFisher) per the manufacturer's protocol followed by wet transfer onto 0.45 μm nitrocellulose membrane (Bio-Rad, Cat. 1620115). Blots were blocked using 5% Dry Milk in TBS for 30 minutes on a lab rocker at room temperature. Blots were rinsed with TBST and probed with rabbit α-TTR monoclonal antibody (Abcam, Cat. Ab75815) at 1:1000 in TBST. Alpha-1 antitrypsin was used as a loading control (Sigma, Cat. HPA001292) at 1:1000 in TBST and incubated simultaneously with the TTR primary antibody. Blots were sealed in a bag and kept overnight at 4° C. on a lab rocker. After incubation, blots were rinsed 3 times for 5 min each in TBST and probed with secondary antibodies to Rabbit (ThermoFisher, Cat. PISA535571) at 1:25,000 in TBST for 30 min at room temperature. After incubation, blots were rinsed 3 times for 5 min each in TBST and 2 times with PBS. Blots were visualized and analyzed using a Licor Odyssey system.

### Analysis of Intracellular TTR by Western Blot

The hepatocellular carcinoma cell line, HUH7, was transfected as previously described with select guides from Table 1. Six-days post-transfection, the media was removed and the cells were lysed with 50 μL/well RIPA buffer (Boston Bio Products, Cat. BP-115) plus freshly added protease inhibitor mixture consisting of complete protease inhibitor cocktail (Sigma, Cat. 11697498001), 1 mM DTT, and 250 U/ml Benzonase (EMD Millipore, Cat. 71206-3). Cells were kept on ice for 30 minutes at which time NaCl (1 M final concentration) was added. Cell lysates were thoroughly mixed and retained on ice for 30 minutes. The whole cell extracts (“WCE”) were transferred to a PCR plate and centrifuged to pellet debris. A Bradford assay (Bio-Rad, Cat. 500-0001) was used to assess protein content of the lysates. The Bradford assay procedure was completed per the manufacturer's protocol. Extracts were stored at minus 20° C. prior to use. Western blots were performed to assess intracellular TTR protein levels. Lysates were mixed with Laemmli buffer and denatured at 95° C. for 10 min. Western blots were run using the NuPage system on 12% Bis-Tris gels (ThermoFisher) per the manufacturer's protocol followed by wet transfer onto 0.45 μm nitrocellulose membrane (Bio-Rad, Cat. 1620115). After transfer membranes were rinsed thoroughly with water and stained with Ponceau S solution (Boston Bio Products, Cat. ST-180) to confirm complete and even transfer. Blots were blocked using 5% Dry Milk in TBS for 30 minutes on a lab rocker at room temperature. Blots were rinsed with TBST and probed with rabbit α-TTR monoclonal antibody (Abcam, Cat. Ab75815) at 1:1000 in TBST. (3-actin was used as a loading control (ThermoFisher, Cat. AM4302) at 1:2500 in TBST and incubated simultaneously with the TTR primary antibody. Blots were sealed in a bag and kept overnight at 4° C. on a lab rocker. After incubation, blots were rinsed 3 times for 5 minutes each in TBST and probed with secondary antibodies to Mouse and Rabbit (ThermoFisher, Cat. PI35518 and PISA535571) at 1:25,000 each in TBST for 30 min at room temperature. After incubation, blots were rinsed 3 times for 5 min each in TBST and 2 times with PBS. Blots were visualized and analyzed using a Licor Odyssey system.

### Example 2. Screening of dgRNA Sequences

Cross Screening of TTR dgRNAs in Multiple Cell Types

Guides in dgRNA format targeting human TTR and the cynomologus matched sequences were delivered to HEK293_Cas9, HUH7 and HepG2 cell lines, as well as primary human hepatocytes and primary cynomolgus monkey hepatocytes as described in Example 1. Percent editing was determined for crRNAs comprising each guide sequence across each cell type and the guide sequences were then rank ordered based on highest % edit. The screening data for the guide sequences in Table 1 in all five cell lines are listed below (Table 4 through 11).

Table 6 shows the average and standard deviation for % Edit, % Insertion (Ins), and % Deletion (Del) for the TTR crRNAs in the human kidney adenocarcinoma cell line, HEK293_Cas9, which constitutively over expresses Spy Cas9 protein.

TABLE 6
TTR editing data in Hek_Cas9 cells transfected with dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR003335 | 26.59 | 4.73 | 4.73 | 0.65 | 21.87 | 4.09 |
| CR003336 | 29.09 | 4.57 | 3.31 | 0.24 | 25.78 | 4.32 |
| CR003337 | 42.72 | 1.72 | 5.24 | 1.62 | 37.48 | 0.70 |
| CR003338 | 52.42 | 3.28 | 4.76 | 0.03 | 47.66 | 3.30 |
| CR003339 | 56.37 | 4.13 | 49.39 | 3.23 | 6.98 | 0.91 |
| CR003340 | 42.38 | 8.43 | 27.88 | 4.31 | 14.50 | 4.13 |
| CR003341 | 20.04 | 5.26 | 6.73 | 1.86 | 13.31 | 3.41 |
| CR003342 | 36.57 | 5.80 | 1.19 | 0.22 | 35.38 | 5.59 |
| CR003343 | 24.36 | 1.51 | 4.82 | 0.43 | 19.53 | 1.39 |
| CR003344 | 33.87 | 2.93 | 4.32 | 0.58 | 29.54 | 2.37 |
| CR003345 | 35.02 | 7.05 | 19.00 | 3.58 | 16.01 | 3.48 |
| CR003346 | 48.33 | 5.81 | 33.03 | 3.12 | 15.30 | 2.72 |
| CR003347 | 21.45 | 5.57 | 0.95 | 0.33 | 20.50 | 5.26 |
| CR003348 | 35.53 | 5.81 | 22.32 | 3.79 | 13.21 | 2.03 |
| CR003349 | 13.19 | 4.46 | 8.03 | 2.81 | 5.16 | 1.66 |
| CR003350 | 22.31 | 4.25 | 5.54 | 0.74 | 16.77 | 3.51 |
| CR003351 | 49.67 | 3.77 | 28.42 | 1.69 | 21.24 | 2.22 |
| CR003352 | 27.90 | 7.55 | 4.91 | 1.35 | 22.99 | 6.26 |
| CR003353 | 25.03 | 5.16 | 3.71 | 0.75 | 21.32 | 4.42 |
| CR003354 | 18.46 | 2.02 | 2.56 | 0.21 | 15.90 | 1.89 |
| CR003355 | 30.60 | 2.53 | 6.99 | 0.80 | 23.61 | 1.75 |
| CR003356 | 32.21 | 4.71 | 10.03 | 1.39 | 22.19 | 3.36 |
| CR003357 | 43.23 | 6.71 | 5.38 | 0.87 | 37.85 | 5.88 |
| CR003358 | 5.44 | 0.86 | 1.29 | 0.16 | 4.14 | 0.84 |
| CR003359 | 37.75 | 7.50 | 18.35 | 3.73 | 19.40 | 3.78 |
| CR003360 | 22.68 | 3.16 | 2.70 | 0.56 | 19.98 | 2.60 |
| CR003361 | 34.45 | 8.97 | 8.66 | 1.66 | 25.78 | 7.32 |
| CR003362 | 9.90 | 2.66 | 1.48 | 0.33 | 8.41 | 2.33 |
| CR003363 | 31.03 | 10.74 | 14.77 | 4.21 | 16.26 | 6.54 |
| CR003364 | 35.65 | 7.90 | 19.17 | 4.24 | 16.48 | 3.76 |
| CR003365 | 36.43 | 6.20 | 11.83 | 1.88 | 24.61 | 4.45 |
| CR003366 | 47.36 | 6.59 | 10.10 | 1.28 | 37.26 | 5.32 |
| CR003367 | 47.11 | 15.43 | 28.44 | 9.11 | 18.67 | 6.33 |
| CR003368 | 40.35 | 10.13 | 3.73 | 0.96 | 36.61 | 9.17 |
| CR003369 | 33.10 | 7.26 | 9.06 | 1.12 | 24.04 | 6.16 |
| CR003370 | 34.22 | 5.69 | 4.49 | 0.67 | 29.73 | 5.06 |
| CR003371 | 25.60 | 8.33 | 3.84 | 1.41 | 21.76 | 6.92 |
| CR003372 | 15.24 | 7.92 | 3.25 | 1.61 | 11.99 | 6.31 |
| CR003373 | 13.55 | 2.40 | 1.31 | 0.21 | 12.25 | 2.19 |
| CR003374 | 10.91 | 0.88 | 0.81 | 0.10 | 10.10 | 0.81 |
| CR003375 | 11.63 | 3.18 | 0.78 | 0.17 | 10.85 | 3.05 |
| CR003376 | 28.16 | 4.49 | 1.35 | 0.18 | 26.81 | 4.52 |
| CR003377 | 24.70 | 4.44 | 2.71 | 0.54 | 21.99 | 3.91 |
| CR003378 | 20.97 | 2.67 | 4.49 | 0.49 | 16.48 | 2.18 |
| CR003379 | 26.32 | 2.91 | 5.34 | 0.61 | 20.98 | 2.30 |
| CR003380 | 47.64 | 5.74 | 3.64 | 0.24 | 44.00 | 5.52 |
| CR003381 | 22.04 | 5.74 | 3.82 | 1.26 | 18.23 | 4.64 |
| CR003382 | 29.95 | 3.13 | 4.46 | 0.45 | 25.49 | 2.73 |
| CR003383 | 40.47 | 0.64 | 25.12 | 0.45 | 15.35 | 0.66 |
| CR003384 | 17.45 | 1.32 | 1.45 | 0.23 | 16.00 | 1.42 |
| CR003385 | 26.19 | 5.62 | 7.36 | 1.57 | 18.82 | 4.06 |
| CR003386 | 33.12 | 10.65 | 2.94 | 0.63 | 30.18 | 10.03 |
| CR003387 | 24.68 | 5.93 | 7.75 | 1.99 | 16.92 | 3.94 |
| CR003388 | 19.23 | 4.41 | 1.41 | 0.39 | 17.82 | 4.07 |
| CR003389 | 34.18 | 5.09 | 10.30 | 2.12 | 23.87 | 3.02 |
| CR003390 | 28.02 | 3.77 | 4.31 | 0.25 | 23.71 | 3.61 |
| CR003391 | 44.81 | 4.67 | 0.61 | 0.07 | 44.19 | 4.63 |
| CR003392 | 21.67 | 7.52 | 0.85 | 0.26 | 20.82 | 7.27 |

Table 7 shows the average and standard deviation for % Edit, % Insertion (Ins), and % Deletion (Del) for the tested TTR crRNAs co-transfected with Spy Cas9 mRNA (SEQ ID NO:2) in the human hepatocellular carcinoma cell line, HUH7.

TABLE 7
TTR editing data in HUH7 cells transfected
with Spy Cas9 mRNA and dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR003335 | 31.95 | 4.50 | 4.62 | 0.83 | 27.57 | 4.08 |
| CR003336 | 30.05 | 4.25 | 4.14 | 1.07 | 26.56 | 3.55 |
| CR003337 | 55.72 | 3.12 | 8.34 | 0.93 | 48.95 | 2.24 |
| CR003338 | 75.64 | 2.03 | 10.22 | 1.42 | 67.06 | 2.79 |
| CR003339 | 79.97 | 4.73 | 60.55 | 3.94 | 20.13 | 1.02 |
| CR003340 | 46.93 | 7.12 | 33.33 | 6.01 | 14.23 | 1.65 |
| CR003341 | 20.58 | 5.98 | 7.78 | 1.64 | 13.20 | 4.44 |
| CR003342 | 45.14 | 7.16 | 1.23 | 0.91 | 44.66 | 7.68 |
| CR003343 | 76.13 | 7.04 | 9.58 | 3.49 | 66.97 | 6.10 |
| CR003344 | 64.02 | 3.33 | 10.76 | 1.35 | 54.40 | 2.71 |
| CR003345 | 72.43 | 2.17 | 41.33 | 0.96 | 32.18 | 1.37 |
| CR003346 | 18.07 | 1.02 | 13.17 | 1.39 | 6.97 | 3.06 |
| CR003347 | 32.16 | 5.50 | 1.64 | 0.42 | 30.79 | 5.11 |
| CR003348 | 57.14 | 10.98 | 36.08 | 6.97 | 22.71 | 4.42 |
| CR003349 | 14.14 | 4.99 | 9.73 | 3.26 | 4.82 | 1.91 |
| CR003350 | 52.91 | 7.61 | 13.43 | 2.00 | 41.64 | 6.03 |
| CR003351 | 63.51 | 4.61 | 36.87 | 2.49 | 27.49 | 2.14 |
| CR003352 | 39.68 | 9.53 | 7.62 | 7.42 | 32.79 | 7.37 |
| CR003353 | 69.18 | 4.59 | 7.73 | 2.46 | 62.87 | 3.13 |
| CR003354 | 12.27 | 3.38 | 1.25 | 0.40 | 11.46 | 3.23 |
| CR003355 | 38.83 | 5.31 | 9.40 | 1.81 | 30.31 | 3.56 |
| CR003356 | 49.63 | 5.55 | 18.98 | 2.67 | 31.31 | 3.04 |
| CR003357 | 36.31 | 5.72 | 6.37 | 1.17 | 30.82 | 4.68 |
| CR003358 | 36.50 | 6.17 | 10.53 | 1.56 | 26.60 | 4.49 |
| CR003359 | 66.75 | 5.84 | 21.73 | 2.30 | 45.97 | 3.93 |
| CR003360 | 58.62 | 8.73 | 5.01 | 0.60 | 55.13 | 8.19 |
| CR003361 | 28.68 | 6.52 | 6.84 | 1.26 | 22.44 | 5.31 |
| CR003362 | 26.43 | 0.83 | 3.43 | 0.32 | 23.76 | 0.85 |
| CR003363 | 41.01 | 7.16 | 17.83 | 3.32 | 23.78 | 3.97 |
| CR003364 | 47.13 | 10.61 | 24.68 | 5.15 | 23.03 | 5.74 |
| CR003365 | 60.68 | 5.25 | 17.77 | 1.57 | 43.82 | 3.73 |
| CR003366 | 69.98 | 8.84 | 20.77 | 3.10 | 50.32 | 5.69 |
| CR003367 | 66.29 | 4.48 | 33.62 | 4.14 | 33.48 | 0.51 |
| CR003368 | 31.57 | 11.73 | 3.08 | 0.92 | 29.69 | 11.32 |
| CR003369 | 24.19 | 6.89 | 7.12 | 2.27 | 17.38 | 4.76 |
| CR003370 | 39.16 | 11.59 | 4.83 | 1.79 | 35.55 | 10.35 |
| CR003371 | 40.47 | 7.68 | 6.07 | 0.89 | 35.65 | 7.01 |
| CR003372 | 21.52 | 6.02 | 4.89 | 1.66 | 17.25 | 4.58 |
| CR003373 | 27.29 | 4.45 | 3.31 | 0.66 | 25.12 | 4.12 |
| CR003374 | 3.10 | 0.68 | 0.45 | 0.24 | 2.87 | 0.54 |
| CR003375 | 2.38 | 0.22 | 0.26 | 0.14 | 2.25 | 0.12 |
| CR003376 | 19.42 | 5.60 | 1.37 | 0.45 | 18.55 | 5.28 |
| CR003377 | 34.93 | 5.47 | 5.59 | 0.88 | 29.89 | 4.71 |
| CR003378 | 40.73 | 4.63 | 9.73 | 1.85 | 32.27 | 2.91 |
| CR003379 | 19.18 | 5.17 | 3.38 | 0.77 | 16.48 | 4.32 |
| CR003380 | 31.76 | 5.81 | 3.29 | 0.57 | 29.29 | 5.42 |
| CR003381 | 99.70 | 0.17 | 1.92 | 0.20 | 99.70 | 0.17 |
| CR003382 | 34.47 | 5.71 | 0.14 | 0.16 | 34.47 | 5.71 |
| CR003383 | 42.89 | 10.14 | 2.14 | 0.56 | 41.19 | 9.67 |
| CR003384 | 17.03 | 1.95 | 0.84 | 0.30 | 16.29 | 1.84 |
| CR003386 | 69.40 | 19.41 | 0.53 | 0.23 | 69.34 | 19.32 |
| CR003387 | 25.64 | 3.69 | 0.23 | 0.07 | 25.55 | 3.62 |
| CR003388 | 59.48 | 4.29 | 3.88 | 0.68 | 56.45 | 4.45 |
| CR003389 | 62.32 | 1.97 | 13.19 | 1.18 | 50.90 | 1.02 |
| CR003390 | 18.97 | 4.82 | 3.31 | 0.91 | 16.49 | 3.98 |
| CR003391 | 61.31 | 13.21 | 2.10 | 0.51 | 59.70 | 12.76 |
| CR003392 | 28.37 | 8.58 | 1.93 | 0.73 | 26.98 | 7.94 |

Table 8 shows the average and standard deviation for % Edit, % Insertion (Ins), and % Deletion (Del) for the tested TTR and control crRNAs co-transfected with Spy Cas9 mRNA (SEQ ID NO:2) in the human hepatocellular carcinoma cell line, HepG2.

TABLE 8
TTR editing data in HepG2 cells transfected
with Spy Cas9 mRNA and dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR001261 | 49.16 | 7.45 | 16.46 | 3.46 | 32.71 | 4.06 |
| (control) | | | | | | |
| CR001262 | 63.33 | 5.66 | 59.88 | 4.92 | 3.45 | 0.86 |
| (control) | | | | | | |
| CR001263 | 39.19 | 6.98 | 37.59 | 8.01 | 1.60 | 1.92 |
| (control) | | | | | | |
| CR001264 | 57.09 | 12.14 | 47.47 | 9.25 | 9.61 | 2.89 |
| (control) | | | | | | |
| CR003335 | 37.19 | 2.12 | 32.96 | 1.67 | 4.23 | 0.59 |
| CR003336 | 31.31 | 5.47 | 30.48 | 5.10 | 0.83 | 0.75 |
| CR003337 | 61.93 | 2.68 | 59.28 | 2.11 | 2.65 | 1.39 |
| CR003338 | 68.00 | 6.09 | 65.40 | 6.78 | 2.60 | 1.17 |
| CR003339 | 68.21 | 7.67 | 12.37 | 1.47 | 55.84 | 6.31 |
| CR003340 | 37.76 | 6.01 | 6.12 | 1.95 | 31.65 | 4.07 |
| CR003341 | 15.60 | 5.49 | 9.94 | 3.38 | 5.66 | 2.13 |
| CR003342 | 11.06 | 6.71 | 10.78 | 6.69 | 0.28 | 0.03 |
| CR003343 | 45.41 | 15.20 | 40.05 | 10.79 | 5.36 | 5.20 |
| CR003344 | 33.43 | 6.11 | 29.81 | 5.09 | 3.62 | 1.13 |
| CR003345 | 10.58 | 9.25 | 6.12 | 5.38 | 4.45 | 3.87 |
| CR003346 | 0.13 | 0.05 | 0.07 | 0.02 | 0.05 | 0.03 |
| CR003347 | 22.57 | 10.94 | 21.08 | 11.19 | 1.49 | 0.90 |
| CR003348 | 38.44 | 10.45 | 17.04 | 5.04 | 21.40 | 5.89 |
| CR003349 | 8.36 | 2.19 | 4.46 | 1.75 | 3.91 | 0.76 |
| CR003350 | 29.60 | 5.17 | 25.16 | 4.56 | 4.44 | 0.67 |
| CR003351 | 57.54 | 5.67 | 31.98 | 2.63 | 25.57 | 3.08 |
| CR003352 | 44.28 | 8.71 | 39.51 | 7.10 | 4.77 | 1.79 |
| CR003353 | 60.40 | 11.37 | 56.71 | 9.95 | 3.68 | 1.45 |
| CR003354 | 5.36 | 3.94 | 4.84 | 3.41 | 0.53 | 0.71 |
| CR003355 | 15.80 | 5.38 | 12.36 | 4.23 | 3.44 | 1.16 |
| CR003356 | 9.39 | 1.82 | 5.67 | 1.03 | 3.72 | 0.92 |
| CR003357 | 45.83 | 10.66 | 42.37 | 8.47 | 3.46 | 2.28 |
| CR003358 | 35.93 | 7.34 | 28.66 | 7.76 | 7.27 | 1.77 |
| CR003359 | 64.44 | 14.90 | 48.79 | 14.32 | 15.65 | 1.94 |
| CR003360 | 41.31 | 12.23 | 38.94 | 10.60 | 2.38 | 1.78 |
| CR003361 | 14.05 | 4.79 | 11.47 | 4.35 | 2.58 | 0.43 |
| CR003362 | 17.44 | 4.34 | 16.50 | 4.86 | 0.94 | 0.52 |
| CR003363 | 42.65 | 9.90 | 28.58 | 6.95 | 14.07 | 3.01 |
| CR003364 | 51.88 | 7.67 | 31.03 | 2.67 | 20.85 | 5.03 |
| CR003365 | 46.88 | 15.78 | 35.77 | 13.49 | 11.11 | 2.30 |
| CR003366 | 54.69 | 9.10 | 46.20 | 8.98 | 8.49 | 1.11 |
| CR003367 | 45.55 | 8.19 | 24.28 | 6.57 | 21.27 | 1.62 |
| CR003368 | 51.55 | 8.60 | 48.34 | 9.87 | 3.22 | 1.36 |
| CR003369 | 22.62 | 4.01 | 17.11 | 4.47 | 5.51 | 2.52 |
| CR003370 | 28.51 | 6.94 | 24.88 | 6.17 | 3.62 | 1.45 |
| CR003371 | 15.91 | 4.17 | 14.07 | 4.02 | 1.84 | 0.22 |
| CR003372 | 14.57 | 2.47 | 12.14 | 2.08 | 2.42 | 0.40 |
| CR003373 | 17.69 | 8.41 | 15.92 | 6.44 | 1.77 | 1.97 |
| CR003374 | 5.43 | 0.53 | 5.12 | 0.62 | 0.31 | 0.36 |
| CR003375 | 2.06 | 0.04 | 1.96 | 0.06 | 0.10 | 0.03 |
| CR003376 | 14.41 | 3.01 | 14.16 | 2.93 | 0.24 | 0.10 |
| CR003377 | 16.30 | 2.85 | 15.29 | 2.59 | 1.02 | 0.59 |
| CR003378 | 8.16 | 3.83 | 6.82 | 3.43 | 1.34 | 0.61 |
| CR003379 | 19.74 | 4.24 | 17.70 | 4.30 | 2.04 | 0.33 |
| CR003380 | 17.08 | 2.48 | 14.78 | 1.18 | 2.30 | 1.36 |
| CR003381 | 6.81 | 3.48 | 6.18 | 3.82 | 0.63 | 0.44 |
| CR003382 | 1.73 | 0.14 | 1.58 | 0.12 | 0.15 | 0.03 |
| CR003383 | 6.35 | 1.67 | 6.19 | 1.68 | 0.16 | 0.04 |
| CR003384 | 3.37 | 0.88 | 3.12 | 0.94 | 0.25 | 0.09 |
| CR003385 | 53.94 | 9.41 | 46.32 | 10.66 | 7.62 | 1.29 |
| CR003386 | 2.71 | 0.76 | 2.15 | 0.77 | 0.56 | 0.53 |
| CR003387 | 1.39 | 0.15 | 1.27 | 0.17 | 0.12 | 0.02 |
| CR003388 | 9.33 | 4.47 | 7.76 | 4.56 | 1.56 | 0.10 |
| CR003389 | 31.84 | 6.09 | 27.27 | 5.96 | 4.57 | 1.21 |
| CR003390 | 24.88 | 4.96 | 22.44 | 3.41 | 2.44 | 2.25 |
| CR003391 | 48.78 | 14.41 | 48.28 | 14.44 | 0.50 | 0.52 |
| CR003392 | 14.64 | 5.25 | 14.32 | 4.95 | 0.33 | 0.36 |
| CR005298 | 42.65 | 10.94 | 21.29 | 8.16 | 21.36 | 2.87 |
| CR005299 | 38.61 | 5.57 | 36.32 | 3.99 | 2.30 | 2.11 |
| CR005300 | 64.34 | 9.55 | 53.20 | 6.59 | 11.15 | 3.33 |
| CR005301 | 37.04 | 5.32 | 33.39 | 3.85 | 3.65 | 1.89 |
| CR005302 | 33.21 | 2.19 | 30.93 | 2.43 | 2.29 | 0.24 |
| CR005303 | 21.63 | 6.05 | 20.55 | 5.80 | 1.08 | 0.25 |
| CR005304 | 62.82 | 3.28 | 8.07 | 1.22 | 54.75 | 4.27 |
| CR005305 | 13.51 | 3.58 | 12.30 | 3.49 | 1.21 | 0.84 |
| CR005306 | 24.07 | 5.24 | 21.20 | 5.03 | 2.87 | 1.10 |
| CR005307 | 22.03 | 3.86 | 7.70 | 1.35 | 14.33 | 4.15 |

Table 9 shows the average and standard deviation for % Edit, % Insertion (Ins), and % Deletion (Del) for the tested FIR dgRNAs electroporated with Spy Cas9 protein (RNP) in primary human hepatocytes.

TABLE 9
TTR editing data in primary human hepatocytes electroporated
with Spy Cas9 protein loaded with dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR003335 | 72.20 | 4.53 | 69.70 | 4.36 | 2.50 | 0.30 |
| CR003336 | 39.17 | 3.04 | 38.43 | 3.20 | 0.70 | 0.17 |
| CR003337 | 54.27 | 2.70 | 53.23 | 3.05 | 1.30 | 0.26 |
| CR003338 | 83.03 | 4.84 | 80.87 | 4.63 | 2.13 | 0.25 |
| CR003339 | 43.00 | 2.66 | 8.93 | 1.86 | 34.07 | 1.72 |
| CR003340 | 12.03 | 1.55 | 5.60 | 1.32 | 6.50 | 0.53 |
| CR003341 | 11.43 | 0.71 | 7.03 | 0.50 | 4.40 | 1.21 |
| CR003342 | 32.77 | 3.63 | 31.87 | 3.28 | 0.90 | 0.35 |
| CR003343 | 77.10 | 2.21 | 75.63 | 2.01 | 1.50 | 0.36 |
| CR003344 | 39.40 | 3.86 | 33.30 | 2.52 | 6.10 | 1.31 |
| CR003345 | 48.07 | 6.24 | 34.53 | 2.95 | 13.57 | 3.74 |
| CR003346 | 35.67 | 1.80 | 20.83 | 1.65 | 14.83 | 1.66 |
| CR003347 | 82.30 | 5.93 | 81.97 | 5.98 | 0.43 | 0.15 |
| CR003348 | 28.53 | 1.79 | 11.30 | 2.46 | 17.27 | 0.86 |
| CR003349 | 4.10 | 0.17 | 2.33 | 0.46 | 1.87 | 0.25 |
| CR003350 | 28.13 | 3.50 | 22.40 | 2.41 | 5.73 | 1.22 |
| CR003351 | 51.77 | 5.11 | 30.83 | 3.32 | 20.97 | 2.43 |
| CR003352 | 29.83 | 4.18 | 25.63 | 3.67 | 4.30 | 0.56 |
| CR003353 | 84.83 | 4.68 | 82.23 | 4.05 | 2.63 | 0.74 |
| CR003354 | 2.50 | 0.36 | 2.43 | 0.32 | 0.03 | 0.06 |
| CR003355 | 12.53 | 1.54 | 10.60 | 2.36 | 1.97 | 1.17 |
| CR003356 | 9.97 | 2.68 | 7.80 | 2.01 | 2.23 | 0.85 |
| CR003357 | 36.23 | 4.02 | 35.47 | 4.11 | 0.77 | 0.61 |
| CR003358 | 5.70 | 1.42 | 4.93 | 1.36 | 0.80 | 0.26 |
| CR003359 | 63.77 | 7.07 | 56.33 | 5.81 | 7.50 | 1.35 |
| CR003360 | 32.23 | 3.09 | 31.67 | 2.97 | 0.63 | 0.31 |
| CR003361 | 4.10 | 0.36 | 3.73 | 0.42 | 0.37 | 0.06 |
| CR003362 | 7.03 | 1.30 | 6.87 | 1.20 | 0.20 | 0.20 |
| CR003363 | 9.43 | 8.22 | 7.80 | 6.86 | 1.63 | 1.44 |
| CR003364 | 23.30 | 5.20 | 16.93 | 4.96 | 6.53 | 0.55 |
| CR003365 | 42.37 | 3.88 | 35.57 | 1.88 | 6.83 | 2.00 |
| CR003366 | 34.70 | 3.26 | 31.63 | 2.98 | 3.10 | 1.15 |
| CR003367 | 39.20 | 5.31 | 22.93 | 4.14 | 16.37 | 1.46 |
| CR003368 | 28.47 | 3.29 | 27.63 | 2.90 | 0.80 | 0.66 |
| CR003369 | 3.67 | 1.16 | 3.30 | 1.06 | 0.40 | 0.20 |
| CR003370 | 15.27 | 1.75 | 14.43 | 1.72 | 0.90 | 0.20 |
| CR003371 | 16.20 | 2.13 | 14.47 | 2.37 | 1.87 | 0.81 |
| CR003372 | 12.17 | 2.69 | 10.47 | 2.63 | 1.77 | 0.12 |
| CR003373 | 0.87 | 0.21 | 0.83 | 0.25 | 0.07 | 0.12 |
| CR003374 | 0.80 | 0.17 | 0.70 | 0.26 | 0.10 | 0.10 |
| CR003375 | 1.33 | 1.10 | 1.27 | 1.08 | 0.07 | 0.06 |
| CR003376 | 1.90 | 1.06 | 1.87 | 1.00 | 0.03 | 0.06 |
| CR003377 | 10.23 | 1.53 | 10.13 | 1.51 | 0.10 | 0.10 |
| CR003378 | 4.60 | 1.92 | 3.87 | 1.19 | 0.73 | 0.67 |
| CR003379 | 6.57 | 1.00 | 6.30 | 0.70 | 0.27 | 0.31 |
| CR003380 | 5.37 | 2.57 | 5.27 | 2.54 | 0.10 | 0.10 |
| CR003381 | 6.20 | 2.74 | 5.83 | 2.61 | 0.50 | 0.10 |
| CR003382 | 8.40 | 2.07 | 8.10 | 1.87 | 0.43 | 0.21 |
| CR003383 | 8.57 | 0.75 | 3.37 | 0.67 | 5.27 | 0.46 |
| CR003384 | 1.87 | 0.67 | 1.73 | 0.57 | 0.23 | 0.12 |
| CR003385 | 40.87 | 6.86 | 38.43 | 6.41 | 2.53 | 0.45 |
| CR003386 | 4.90 | 1.20 | 4.47 | 1.14 | 0.47 | 0.25 |
| CR003387 | 1.87 | 0.25 | 1.70 | 0.26 | 0.20 | 0.10 |
| CR003388 | 5.70 | 0.40 | 5.47 | 0.40 | 0.27 | 0.12 |
| CR003389 | 27.67 | 2.76 | 27.20 | 2.88 | 0.50 | 0.36 |
| CR003390 | 15.97 | 3.86 | 15.80 | 3.99 | 0.23 | 0.15 |
| CR003391 | 29.77 | 3.85 | 29.57 | 3.85 | 0.27 | 0.06 |
| CR003392 | 4.13 | 1.21 | 4.00 | 1.15 | 0.17 | 0.06 |
| CR005298 | 39.90 | 2.92 | 22.37 | 3.04 | 17.57 | 0.42 |
| CR005299 | 8.65 | 0.78 | 8.30 | 0.99 | 0.35 | 0.21 |
| CR005300 | 57.47 | 1.69 | 53.47 | 1.86 | 4.10 | 0.92 |
| CR005301 | 25.37 | 1.65 | 24.00 | 2.26 | 1.60 | 0.82 |
| CR005302 | 61.10 | 5.20 | 60.10 | 4.77 | 1.00 | 0.46 |
| CR005303 | 53.57 | 8.52 | 53.07 | 8.36 | 0.53 | 0.47 |
| CR005304 | 67.00 | 5.80 | 5.53 | 1.37 | 61.63 | 6.98 |
| CR005305 | 3.83 | 0.78 | 3.53 | 0.61 | 0.40 | 0.17 |
| CR005306 | 9.43 | 1.63 | 8.07 | 2.17 | 1.37 | 0.72 |
| CR005307 | 8.17 | 1.20 | 5.20 | 0.87 | 3.00 | 0.82 |

Table 10 shows the average and standard deviation for % Edit, % Insertion (Ins), and % Deletion (Del) for the tested FIR and control dgRNAs transfected with Spy Cas9 protein (RNP) in primary human hepatocytes.

TABLE 10
TTR editing data in primary human hepatocytes
transfected with Spy Cas9 loaded with dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR001261 | 32.51 | 1.00 | 12.50 | 0.47 | 20.01 | 0.59 |
| CR001262 | 50.09 | 1.48 | 45.25 | 1.69 | 4.83 | 0.31 |
| CR001263 | 15.25 | 2.41 | 14.83 | 2.37 | 0.42 | 0.10 |
| CR001264 | 45.30 | 3.48 | 23.87 | 2.09 | 21.43 | 1.68 |
| CR003335 | 51.14 | 4.27 | 49.51 | 4.04 | 1.63 | 0.25 |
| CR003336 | 30.70 | 2.41 | 30.11 | 2.48 | 0.58 | 0.11 |
| CR003337 | 49.43 | 4.75 | 47.54 | 4.49 | 1.88 | 0.47 |
| CR003338 | 61.34 | 3.55 | 59.13 | 3.44 | 2.22 | 0.11 |
| CR003339 | 45.06 | 9.83 | 8.85 | 1.65 | 36.21 | 8.34 |
| CR003340 | 10.44 | 2.44 | 5.94 | 1.34 | 4.50 | 1.16 |
| CR003341 | 19.66 | 3.67 | 14.64 | 3.31 | 5.02 | 0.37 |
| CR003342 | 20.66 | 2.55 | 19.85 | 2.54 | 0.81 | 0.15 |
| CR003343 | 43.25 | 4.47 | 41.61 | 4.26 | 1.63 | 0.33 |
| CR003344 | 35.45 | 13.12 | 30.97 | 11.72 | 4.48 | 1.51 |
| CR003345 | 28.90 | 6.33 | 21.00 | 5.23 | 7.91 | 1.81 |
| CR003346 | 4.11 | 1.36 | 2.27 | 0.53 | 1.84 | 0.85 |
| CR003347 | 66.35 | 4.48 | 66.11 | 4.51 | 0.24 | 0.08 |
| CR003348 | 23.18 | 2.16 | 13.74 | 1.17 | 9.44 | 0.99 |
| CR003349 | 10.83 | 1.57 | 9.00 | 1.41 | 1.83 | 0.32 |
| CR003350 | 24.84 | 2.74 | 19.77 | 1.91 | 5.07 | 0.89 |
| CR003351 | 40.28 | 1.31 | 23.92 | 0.70 | 16.36 | 0.78 |
| CR003352 | 30.48 | 1.93 | 27.27 | 2.31 | 3.21 | 0.38 |
| CR003353 | 61.54 | 4.13 | 59.38 | 4.04 | 2.16 | 0.11 |
| CR003354 | 10.31 | 1.47 | 10.07 | 1.50 | 0.23 | 0.11 |
| CR003355 | 19.11 | 0.92 | 17.69 | 0.79 | 1.42 | 0.44 |
| CR003356 | 7.53 | 1.78 | 6.24 | 1.51 | 1.29 | 0.32 |
| CR003357 | 49.35 | 2.53 | 48.45 | 2.54 | 0.90 | 0.13 |
| CR003358 | 31.62 | 5.97 | 25.95 | 5.03 | 5.67 | 1.04 |
| CR003359 | 59.47 | 6.05 | 50.96 | 5.69 | 8.51 | 0.54 |
| CR003360 | 31.47 | 4.12 | 30.27 | 4.21 | 1.19 | 0.22 |
| CR003361 | 13.08 | 1.48 | 12.52 | 1.45 | 0.56 | 0.18 |
| CR003362 | 11.65 | 1.24 | 11.10 | 1.06 | 0.56 | 0.36 |
| CR003363 | 27.65 | 2.84 | 21.47 | 2.39 | 6.18 | 0.61 |
| CR003364 | 35.29 | 3.50 | 23.93 | 2.63 | 11.36 | 1.16 |
| CR003365 | 47.78 | 3.67 | 40.24 | 3.12 | 7.54 | 0.72 |
| CR003366 | 42.74 | 3.41 | 37.95 | 2.88 | 4.79 | 0.60 |
| CR003367 | 31.19 | 4.60 | 16.06 | 2.66 | 15.13 | 1.94 |
| CR003368 | 34.83 | 5.05 | 33.83 | 5.09 | 1.00 | 0.10 |
| CR003369 | 12.98 | 0.26 | 11.67 | 0.21 | 1.31 | 0.11 |
| CR003370 | 20.06 | 1.79 | 18.80 | 1.65 | 1.26 | 0.28 |
| CR003371 | 18.80 | 2.73 | 17.23 | 2.34 | 1.57 | 0.43 |
| CR003372 | 17.56 | 2.26 | 15.74 | 2.16 | 1.81 | 0.10 |
| CR003373 | 3.64 | 0.29 | 3.44 | 0.30 | 0.19 | 0.07 |
| CR003374 | 2.65 | 0.33 | 2.52 | 0.33 | 0.14 | 0.02 |
| CR003375 | 5.04 | 0.66 | 4.93 | 0.66 | 0.11 | 0.01 |
| CR003376 | 5.00 | 1.10 | 4.86 | 1.10 | 0.14 | 0.03 |
| CR003377 | 12.77 | 2.00 | 12.45 | 1.84 | 0.31 | 0.18 |
| CR003378 | 8.66 | 1.90 | 8.24 | 1.74 | 0.42 | 0.19 |
| CR003379 | 16.86 | 2.62 | 16.51 | 2.62 | 0.34 | 0.08 |
| CR003380 | 8.17 | 1.42 | 7.71 | 1.47 | 0.46 | 0.10 |
| CR003381 | 7.15 | 0.73 | 6.88 | 0.67 | 0.27 | 0.07 |
| CR003382 | 2.44 | 0.06 | 2.28 | 0.05 | 0.15 | 0.03 |
| CR003383 | 4.76 | 0.40 | 4.52 | 0.42 | 0.24 | 0.09 |
| CR003384 | 3.56 | 0.26 | 3.39 | 0.26 | 0.17 | 0.01 |
| CR003385 | 41.15 | 6.06 | 38.15 | 5.59 | 3.00 | 0.48 |
| CR003386 | 3.22 | 0.25 | 2.97 | 0.27 | 0.25 | 0.02 |
| CR003387 | 1.79 | 0.11 | 1.68 | 0.09 | 0.11 | 0.04 |
| CR003388 | 5.43 | 1.03 | 4.38 | 1.00 | 1.05 | 0.25 |
| CR003389 | 19.87 | 4.39 | 19.19 | 4.52 | 0.68 | 0.24 |
| CR003390 | 16.09 | 2.84 | 15.85 | 2.91 | 0.24 | 0.09 |
| CR003391 | 34.72 | 8.29 | 34.46 | 8.35 | 0.26 | 0.06 |
| CR003392 | 10.07 | 1.06 | 9.93 | 1.02 | 0.14 | 0.04 |
| CR005298 | 32.07 | 1.02 | 21.12 | 1.02 | 10.95 | 0.15 |
| CR005299 | 19.37 | 0.61 | 18.79 | 0.51 | 0.58 | 0.13 |
| CR005300 | 57.23 | 6.24 | 53.62 | 5.44 | 3.61 | 0.87 |
| CR005301 | 31.37 | 3.02 | 29.53 | 2.88 | 1.84 | 0.15 |
| CR005302 | 48.29 | 5.22 | 47.32 | 5.32 | 0.97 | 0.14 |
| CR005303 | 36.45 | 4.83 | 36.06 | 4.72 | 0.39 | 0.12 |
| CR005304 | 49.45 | 6.85 | 4.32 | 0.31 | 45.13 | 6.74 |
| CR005305 | 7.07 | 1.43 | 6.73 | 1.30 | 0.34 | 0.17 |
| CR005306 | 18.81 | 1.82 | 16.24 | 1.57 | 2.57 | 0.35 |
| CR005307 | 18.73 | 1.68 | 10.18 | 0.92 | 8.55 | 0.88 |

Table 11 shows the average and standard deviation for % Edit, % Insertion (Ins), and % Deletion (Del) for the tested FIR and control dgRNAs co-transfected with Spy Cas9 mRNA (SEQ ID NO. 2) in primary human hepatocytes.

TABLE 11
TTR editing data in primary human hepatocytes
transfected with Spy Cas9 mRNA and dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR001261 | 32.33 | 4.95 | 5.83 | 1.63 | 26.47 | 3.30 |
| CR001262 | 41.50 | 4.71 | 34.43 | 3.31 | 7.13 | 1.42 |
| CR001263 | 10.23 | 3.61 | 9.40 | 3.20 | 0.90 | 0.44 |
| CR001264 | 42.80 | 0.50 | 11.90 | 1.32 | 30.90 | 1.80 |
| CR003335 | 36.43 | 2.98 | 33.03 | 2.31 | 3.40 | 0.70 |
| CR003336 | 16.93 | 3.78 | 16.20 | 3.41 | 0.80 | 0.44 |
| CR003337 | 19.30 | 1.57 | 18.10 | 1.44 | 1.23 | 0.15 |
| CR003338 | 36.30 | 9.55 | 33.73 | 9.27 | 2.73 | 0.49 |
| CR003339 | 36.43 | 1.21 | 2.27 | 0.15 | 34.23 | 1.31 |
| CR003340 | 24.97 | 2.78 | 1.83 | 0.23 | 23.17 | 2.66 |
| CR003341 | 15.83 | 1.38 | 6.80 | 0.53 | 9.07 | 0.81 |
| CR003342 | 22.10 | 1.27 | 20.60 | 0.57 | 1.50 | 0.71 |
| CR003343 | 55.03 | 0.38 | 52.40 | 0.53 | 2.60 | 0.44 |
| CR003344 | 31.50 | 1.30 | 22.40 | 1.31 | 9.20 | 0.10 |
| CR003345 | 50.65 | 2.90 | 32.30 | 1.56 | 18.45 | 1.20 |
| CR003346 | 19.97 | 1.94 | 5.63 | 0.55 | 14.33 | 1.72 |
| CR003347 | 41.47 | 3.59 | 41.33 | 3.63 | 0.17 | 0.06 |
| CR003348 | 18.00 | 0.87 | 2.30 | 0.66 | 15.80 | 0.61 |
| CR003349 | 2.57 | 0.81 | 0.90 | 0.35 | 1.70 | 0.46 |
| CR003350 | 26.63 | 4.25 | 16.33 | 2.45 | 10.33 | 1.75 |
| CR003351 | 26.50 | 1.61 | 10.20 | 0.92 | 16.37 | 0.97 |
| CR003352 | 16.80 | 5.03 | 11.73 | 3.86 | 5.07 | 1.14 |
| CR003353 | 53.73 | 6.01 | 49.50 | 5.82 | 4.43 | 0.75 |
| CR003354 | 2.97 | 0.95 | 2.87 | 0.85 | 0.13 | 0.12 |
| CR003355 | 12.07 | 2.61 | 10.47 | 2.08 | 1.63 | 0.59 |
| CR003356 | 7.27 | 0.72 | 4.70 | 0.53 | 2.67 | 0.21 |
| CR003357 | 25.93 | 4.55 | 25.30 | 4.22 | 0.63 | 0.35 |
| CR003358 | 3.90 | 0.79 | 2.73 | 0.45 | 1.17 | 0.51 |
| CR003359 | 32.93 | 4.34 | 25.67 | 3.25 | 7.33 | 1.24 |
| CR003360 | 14.90 | 4.85 | 14.13 | 4.66 | 0.90 | 0.52 |
| CR003361 | 3.53 | 0.60 | 2.73 | 0.55 | 0.87 | 0.15 |
| CR003362 | 6.60 | 1.47 | 6.17 | 1.45 | 0.47 | 0.21 |
| CR003363 | 16.70 | 1.08 | 11.80 | 0.79 | 4.93 | 0.60 |
| CR003364 | 15.63 | 2.45 | 6.73 | 0.81 | 8.93 | 1.70 |
| CR003365 | 26.90 | 3.05 | 20.23 | 2.02 | 6.67 | 1.16 |
| CR003366 | 24.53 | 1.26 | 20.47 | 1.45 | 4.07 | 0.23 |
| CR003367 | 37.33 | 1.40 | 14.03 | 0.40 | 23.37 | 1.25 |
| CR003368 | 11.10 | 1.91 | 10.53 | 1.90 | 0.60 | 0.10 |
| CR003369 | 1.60 | 0.46 | 0.90 | 0.20 | 0.70 | 0.36 |
| CR003370 | 2.83 | 0.57 | 2.33 | 0.40 | 0.50 | 0.17 |
| CR003371 | 3.40 | 0.80 | 2.67 | 0.75 | 0.73 | 0.15 |
| CR003372 | 1.77 | 0.75 | 1.13 | 0.57 | 0.63 | 0.23 |
| CR003373 | 1.40 | 0.36 | 1.00 | 0.35 | 0.37 | 0.12 |
| CR003374 | 0.27 | 0.21 | 0.27 | 0.21 | 0.03 | 0.06 |
| CR003375 | 1.27 | 0.64 | 1.23 | 0.58 | 0.03 | 0.06 |
| CR003376 | 2.83 | 0.81 | 2.73 | 0.81 | 0.13 | 0.06 |
| CR003377 | 17.53 | 6.35 | 16.97 | 6.11 | 0.57 | 0.25 |
| CR003378 | 9.80 | 1.37 | 8.50 | 1.21 | 1.37 | 0.15 |
| CR003379 | 13.20 | 1.18 | 12.00 | 1.05 | 1.27 | 0.15 |
| CR003380 | 2.93 | 0.58 | 2.47 | 0.57 | 0.47 | 0.15 |
| CR003381 | 4.07 | 1.21 | 3.33 | 0.96 | 0.73 | 0.25 |
| CR003382 | 0.97 | 0.25 | 0.97 | 0.25 | 0.00 | 0.00 |
| CR003383 | 15.70 | 3.22 | 2.07 | 0.35 | 13.70 | 2.82 |
| CR003384 | 1.70 | 0.62 | 1.50 | 0.56 | 0.20 | 0.10 |
| CR003385 | 36.77 | 0.70 | 33.23 | 0.74 | 3.60 | 0.26 |
| CR003386 | 8.27 | 1.63 | 8.20 | 1.57 | 0.13 | 0.06 |
| CR003387 | 7.87 | 1.58 | 7.80 | 1.64 | 0.03 | 0.06 |
| CR003388 | 12.97 | 1.30 | 11.87 | 1.21 | 1.17 | 0.25 |
| CR003389 | 44.27 | 1.72 | 41.47 | 1.59 | 2.83 | 0.15 |
| CR003390 | 20.23 | 2.08 | 18.73 | 1.92 | 1.60 | 0.17 |
| CR003391 | 15.47 | 5.87 | 15.20 | 5.72 | 0.30 | 0.10 |
| CR003392 | 2.43 | 0.55 | 2.37 | 0.59 | 0.07 | 0.06 |
| CR005298 | 15.70 | 2.79 | 4.13 | 0.87 | 11.60 | 2.00 |
| CR005299 | 9.43 | 0.68 | 8.93 | 0.68 | 0.60 | 0.00 |
| CR005300 | 31.53 | 3.44 | 27.60 | 2.77 | 3.97 | 0.76 |
| CR005301 | 6.77 | 1.44 | 5.47 | 0.96 | 1.40 | 0.61 |
| CR005302 | 34.80 | 7.17 | 33.67 | 7.01 | 1.13 | 0.21 |
| CR005303 | 35.50 | 5.90 | 35.00 | 5.81 | 0.50 | 0.10 |
| CR005304 | 45.27 | 4.71 | 0.83 | 0.15 | 44.47 | 4.57 |
| CR005305 | 7.53 | 1.06 | 5.93 | 1.10 | 1.60 | 0.10 |
| CR005306 | 9.97 | 0.38 | 7.13 | 0.23 | 2.87 | 0.12 |
| CR005307 | 12.90 | 2.43 | 3.67 | 0.61 | 9.30 | 1.80 |

Table 12 shows the average and standard deviation for % Edit, % Insertion (Ins), and % Deletion (Del) for the tested FIR dgRNAs electroporated with Spy Cas9 protein (RNP) in primary cyno hepatocytes.

TABLE 12
TTR editing data in primary cyno hepatocytes electroporated
with Spy Cas9 protein and dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR003336 | 8.18 | 1.93 | 8.10 | 1.94 | 0.07 | 0.01 |
| CR003337 | 24.94 | 5.80 | 24.10 | 4.71 | 0.84 | 1.10 |
| CR003338 | 44.94 | 9.99 | 44.89 | 9.97 | 0.05 | 0.01 |
| CR003339 | 8.95 | 0.89 | 4.93 | 0.64 | 4.02 | 0.25 |
| CR003340 | 12.53 | 2.22 | 7.72 | 0.13 | 4.80 | 2.09 |
| CR003341 | 8.43 | 10.53 | 7.66 | 9.91 | 0.77 | 0.63 |
| CR003344 | 35.72 | 4.67 | 33.81 | 5.29 | 1.91 | 0.61 |
| CR003345 | 52.92 | 3.26 | 30.74 | 0.78 | 22.19 | 2.48 |
| CR003346 | 1.91 | 0.86 | 1.82 | 0.82 | 0.09 | 0.04 |
| CR003347 | 72.41 | 0.38 | 72.15 | 0.73 | 0.25 | 0.34 |
| CR003352 | 1.25 | 0.20 | 1.16 | 0.21 | 0.09 | 0.01 |
| CR003353 | 4.75 | 0.43 | 4.67 | 0.47 | 0.08 | 0.04 |
| CR003358 | 20.47 | 0.30 | 19.01 | 0.51 | 1.46 | 0.21 |
| CR003359 | 46.17 | 1.14 | 40.66 | 2.00 | 5.51 | 0.86 |
| CR003360 | 29.47 | 0.63 | 29.05 | 1.00 | 0.42 | 0.37 |
| CR003361 | 4.53 | 0.14 | 4.46 | 0.18 | 0.08 | 0.04 |
| CR003362 | 4.59 | 0.80 | 4.36 | 0.77 | 0.22 | 0.03 |
| CR003363 | 15.64 | 1.92 | 13.24 | 2.65 | 2.39 | 0.73 |
| CR003364 | 19.62 | 2.54 | 14.27 | 2.72 | 5.35 | 0.17 |
| CR003365 | 10.31 | 1.81 | 9.33 | 1.80 | 0.97 | 0.01 |
| CR003366 | 18.52 | 0.71 | 17.62 | 0.33 | 0.90 | 0.39 |
| CR003368 | 18.56 | 3.89 | 18.30 | 3.77 | 0.26 | 0.11 |
| CR003369 | 1.53 | 0.25 | 1.28 | 0.40 | 0.25 | 0.15 |
| CR003370 | 2.52 | 0.64 | 2.40 | 0.63 | 0.12 | 0.01 |
| CR003371 | 1.83 | 0.38 | 1.69 | 0.41 | 0.14 | 0.03 |
| CR003372 | 2.15 | 0.30 | 1.83 | 0.33 | 0.32 | 0.04 |
| CR003382 | 10.86 | 2.04 | 8.54 | 1.93 | 2.33 | 0.11 |
| CR003383 | 8.86 | 2.30 | 4.31 | 0.69 | 4.55 | 1.61 |
| CR003384 | 3.75 | 0.35 | 2.50 | 0.37 | 1.25 | 0.02 |
| CR003385 | 30.96 | 1.61 | 26.84 | 2.20 | 4.12 | 0.59 |
| CR003386 | 5.54 | 1.42 | 3.51 | 1.26 | 2.03 | 0.15 |
| CR003387 | 4.72 | 0.03 | 4.55 | 0.08 | 0.17 | 0.11 |
| CR003388 | 6.81 | 0.17 | 6.59 | 0.28 | 0.22 | 0.11 |
| CR003389 | 18.83 | 4.99 | 18.05 | 4.92 | 0.78 | 0.07 |
| CR003390 | 16.87 | 3.88 | 16.49 | 3.48 | 0.39 | 0.39 |
| CR003391 | 36.44 | 1.09 | 35.73 | 1.37 | 0.71 | 0.28 |
| CR003392 | 7.02 | 0.97 | 6.63 | 0.59 | 0.38 | 0.37 |
| CR005299 | 13.48 | 2.96 | 13.23 | 2.74 | 0.26 | 0.22 |
| CR005301 | 46.76 | 1.75 | 46.34 | 2.19 | 0.42 | 0.44 |
| CR005302 | 1.34 | 0.19 | 1.26 | 0.19 | 0.08 | 0.00 |
| CR005303 | 59.28 | 1.05 | 58.72 | 1.06 | 0.56 | 0.00 |
| CR005305 | 11.28 | 0.39 | 11.13 | 0.39 | 0.15 | 0.00 |
| CR005307 | 4.56 | 0.71 | 2.01 | 0.49 | 2.55 | 0.21 |

Table 13 shows the average and standard deviation for % Edit, % Insertion (Ins), and % Deletion (Del) for the tested cyno specific TTR dgRNAs electroporated with Spy Cas9 protein (RNP) on primary cyno hepatocytes.

TABLE 13
TTR editing data in primary cyno hepatocytes electroporated
with Spy Cas9 protein and cyno specific dgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| CR000689 | 24.41 | 1.67 | 18.11 | 2.41 | 6.30 | 0.93 |
| CR005364 | 27.70 | 0.74 | 0.58 | 0.29 | 27.11 | 0.60 |
| CR005365 | 64.94 | 2.03 | 0.10 | 0.04 | 64.85 | 2.05 |
| CR005366 | 77.00 | 1.17 | 0.33 | 0.27 | 76.67 | 0.99 |
| CR005367 | 50.79 | 0.53 | 0.53 | 0.25 | 50.26 | 0.36 |
| CR005368 | 27.60 | 2.07 | 0.33 | 0.45 | 27.27 | 2.32 |
| CR005369 | 42.01 | 0.33 | 8.09 | 0.55 | 33.92 | 0.31 |
| CR005370 | 63.52 | 3.21 | 0.59 | 0.33 | 62.93 | 2.88 |
| CR005371 | 8.42 | 0.69 | 0.31 | 0.12 | 8.10 | 0.57 |
| CR005372 | 17.98 | 1.39 | 0.83 | 0.77 | 17.16 | 0.71 |

### Example 3. Screening of sgRNA Sequences

Cross Screening of TTR sgRNAs in Multiple Cell Types

Guides in modified sgRNA format targeting human and/or cyno TTR were delivered to primary human hepatocytes and primary cyno hepatocytes as described in Example 1. Percent editing was determined for crRNAs comprising each guide sequence across each cell type and the guide sequences were then rank ordered based on highest % edit. The screening data for the guide sequences in Table 2 in both cell lines are listed below (Table 14 through 16).

Table 14 shows the average and standard deviation for % Edit, % Insertion (Ins), and % Deletion (Del) for the tested TTR sgRNAs transfected with Spy Cas9 protein (RNP) in primary human hepatocytes.

TABLE 14
TTR editing data in primary human hepatocytes
transfected with Spy Cas9 protein and sgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| G000480 | 81.80 | 1.98 | 77.15 | 2.19 | 4.70 | 0.28 |
| G000481 | 46.90 | 1.71 | 27.77 | 3.88 | 19.43 | 4.76 |
| G000482 | 66.67 | 2.35 | 56.57 | 4.14 | 10.10 | 1.85 |
| G000483 | 47.90 | 6.56 | 19.57 | 3.37 | 28.50 | 3.25 |
| G000484 | 62.97 | 0.90 | 29.23 | 0.21 | 33.83 | 0.95 |
| G000485 | 56.07 | 3.37 | 53.07 | 2.84 | 3.13 | 0.60 |
| G000486 | 69.73 | 6.86 | 9.83 | 1.93 | 59.93 | 5.63 |
| G000487 | 67.30 | 2.75 | 65.27 | 3.41 | 2.07 | 1.06 |
| G000488 | 61.27 | 1.95 | 26.30 | 1.55 | 35.00 | 1.30 |
| G000489 | 60.17 | 2.75 | 51.07 | 3.18 | 9.43 | 0.45 |
| G000490 | 55.90 | 7.88 | 46.13 | 7.55 | 9.80 | 0.69 |
| G000491 | 74.30 | 1.55 | 70.27 | 2.37 | 4.33 | 0.72 |
| G000492 | 60.97 | 5.81 | 57.90 | 4.64 | 3.13 | 1.35 |
| G000493 | 41.40 | 3.08 | 38.90 | 3.29 | 2.67 | 0.35 |
| G000494 | 62.23 | 3.30 | 61.47 | 3.25 | 0.77 | 0.31 |
| G000495 | 50.80 | 1.85 | 45.80 | 1.25 | 5.37 | 0.64 |
| G000496 | 72.33 | 1.63 | 44.73 | 2.14 | 27.67 | 1.46 |
| G000497 | 59.67 | 1.40 | 51.10 | 1.14 | 8.73 | 0.71 |
| G000498 | 72.80 | 3.75 | 60.17 | 3.12 | 12.70 | 0.72 |
| G000499 | 66.40 | 3.55 | 65.23 | 3.72 | 1.17 | 0.38 |
| G000500 | 65.53 | 1.21 | 62.00 | 1.11 | 3.83 | 0.40 |
| G000501 | 60.93 | 1.91 | 55.13 | 1.43 | 6.00 | 0.56 |

Table 15 shows the average and standard deviation at 12.5 nM for % Edit, % Insertion (Ins), and % Deletion (Del) for the tested FIR sgRNAs co-transfected with Spy Cas9 mRNA (SEQ ID NO:2) in primary human hepatocytes.

TABLE 15
TTR editing data in primary human hepatocytes
transfected with Spy Cas9 mRNA and sgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| G000480 | 73.28 | 0.61 | 59.85 | 0.13 | 13.47 | 0.51 |
| G000481 | 34.30 | 5.26 | 14.62 | 2.59 | 19.77 | 2.72 |
| G000482 | 40.93 | 3.95 | 27.70 | 2.92 | 13.25 | 0.97 |
| G000483 | 27.82 | 2.93 | 4.05 | 0.51 | 23.85 | 2.43 |
| G000484 | 43.37 | 6.79 | 13.98 | 2.61 | 29.48 | 4.15 |
| G000485 | 30.82 | 5.76 | 28.87 | 5.50 | 1.97 | 0.28 |
| G000486 | 59.13 | 5.62 | 2.82 | 0.86 | 56.37 | 4.92 |
| G000487 | 49.57 | 0.99 | 47.38 | 0.89 | 2.27 | 0.24 |
| G000488 | 49.40 | 5.05 | 11.98 | 1.40 | 37.48 | 3.68 |
| G000489 | 24.25 | 2.82 | 14.17 | 2.01 | 10.28 | 1.38 |
| G000490 | 24.72 | 2.35 | 19.38 | 2.04 | 5.38 | 0.41 |
| G000491 | 45.93 | 1.22 | 42.42 | 1.06 | 3.60 | 0.33 |
| G000492 | 34.65 | 2.21 | 32.45 | 2.01 | 2.22 | 0.25 |
| G000493 | 11.55 | 1.35 | 10.65 | 1.58 | 0.97 | 0.30 |
| G000494 | 26.22 | 4.03 | 25.17 | 3.89 | 1.07 | 0.15 |
| G000495 | 47.77 | 1.88 | 43.40 | 1.91 | 4.45 | 0.17 |
| G000496 | 63.30 | 2.60 | 11.08 | 2.10 | 52.25 | 0.67 |
| G000497 | 40.33 | 3.32 | 34.48 | 2.71 | 5.85 | 0.61 |
| G000498 | 60.02 | 5.42 | 45.20 | 4.34 | 14.90 | 1.08 |
| G000499 | 39.30 | 6.04 | 38.58 | 5.86 | 0.77 | 0.12 |
| G000500 | 35.50 | 0.61 | 32.47 | 0.49 | 3.10 | 0.18 |
| G000501 | 40.32 | 1.50 | 33.82 | 2.04 | 6.62 | 0.55 |
| G000567 | 27.28 | 7.59 | 17.35 | 4.72 | 10.02 | 2.94 |
| G000568 | 43.75 | 5.83 | 43.00 | 5.81 | 0.80 | 0.18 |
| G000570 | 68.42 | 3.64 | 68.08 | 3.61 | 0.35 | 0.00 |
| G000571 | 20.47 | 3.41 | 14.47 | 2.72 | 6.13 | 0.78 |
| G000572 | 55.42 | 8.13 | 41.62 | 6.48 | 13.85 | 1.60 |

Table 16 shows the average and standard deviation for % Edit, % Insertion (Ins), and % Deletion (Del) for the tested FIR sgRNAs electroporated with Spy Cas9 protein (RNP) on primary cyno hepatocytes. Note that guides G000480 and G000488 have one mismatch to cyno, which may compromise their editing efficiency in cyno cells.

TABLE 16
TTR editing data in primary cyno hepatocytes electroporated
with Spy Cas9 protein and sgRNAs

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| G000480 | 10.20 | 0.56 | 9.83 | 0.81 | 0.37 | 0.25 |
| G000481 | 69.13 | 8.62 | 33.73 | 2.67 | 35.50 | 11.23 |
| G000482 | 75.17 | 2.34 | 55.23 | 2.00 | 20.03 | 0.85 |
| G000485 | 22.93 | 0.95 | 22.00 | 0.82 | 1.07 | 0.21 |
| G000486 | 79.90 | 0.79 | 11.90 | 0.85 | 68.07 | 0.35 |
| G000488 | 9.63 | 0.50 | 5.37 | 0.38 | 4.27 | 0.35 |
| G000489 | 67.53 | 1.15 | 53.53 | 1.56 | 14.17 | 0.64 |
| G000490 | 61.67 | 0.72 | 54.47 | 1.10 | 7.27 | 1.23 |
| G000491 | 66.20 | 1.11 | 64.37 | 0.47 | 1.90 | 0.70 |
| G000493 | 50.13 | 0.74 | 48.07 | 1.69 | 2.10 | 0.98 |
| G000494 | 81.53 | 0.71 | 79.57 | 0.49 | 2.07 | 0.67 |
| G000498 | 91.37 | 1.48 | 68.50 | 1.64 | 22.87 | 1.50 |
| G000499 | 83.40 | 0.36 | 82.00 | 0.20 | 1.43 | 0.55 |
| G000500 | 45.20 | 3.66 | 42.60 | 3.80 | 2.63 | 0.25 |

Table 17 shows the average and standard deviation for % Edit, % Insertion (Ins), and % Deletion (Del) for the tested cyno specific TTR sgRNAs electroporated with Spy Cas9 protein (RNP) on primary cyno hepatocytes.

TABLE 17
TTR editing data in primary cyno hepatocytes electroporated
with Spy Cas9 protein and cyno specific sgRNAs (e.g.,
those having an analogous human gRNA, See Table 3)

| | Avg | Std | Avg | Std | Avg | Std |
|---|---|---|---|---|---|---|
| | % | Dev % | % | Dev % | % | Dev % |
| GUIDE ID | Edit | Edit | Insert | Insert | Deletion | Deletion |
| G000502 | 95.10 | 0.96 | 13.97 | 1.69 | 81.27 | 2.60 |
| G000503 | 58.53 | 2.40 | 52.07 | 1.68 | 6.50 | 2.46 |
| G000504 | 77.17 | 0.96 | 69.73 | 1.29 | 7.53 | 0.57 |
| G000505 | 95.53 | 1.06 | 95.50 | 1.01 | 0.10 | 0.10 |
| G000506 | 89.43 | 1.36 | 86.90 | 1.64 | 3.07 | 0.42 |
| G000507 | 71.17 | 3.22 | 67.03 | 2.39 | 4.60 | 1.65 |
| G000508 | 45.63 | 3.01 | 41.57 | 2.95 | 4.17 | 0.91 |
| G000509 | 93.03 | 0.81 | 43.60 | 1.30 | 49.73 | 1.76 |
| G000510 | 90.80 | 0.53 | 89.13 | 0.40 | 1.77 | 0.12 |
| G000511 | 62.77 | 1.63 | 60.87 | 1.55 | 2.00 | 0.35 |

### Example 4. Screening of Lipid Nanoparticle (LNP) Formulations Containing Spy Ca9 mRNA and sgRNA

Cross screening of LNP formulated TTR sgRNAs with Spy Cas9 mRNA in primary human hepatocytes and primary cyno hepatocytes.

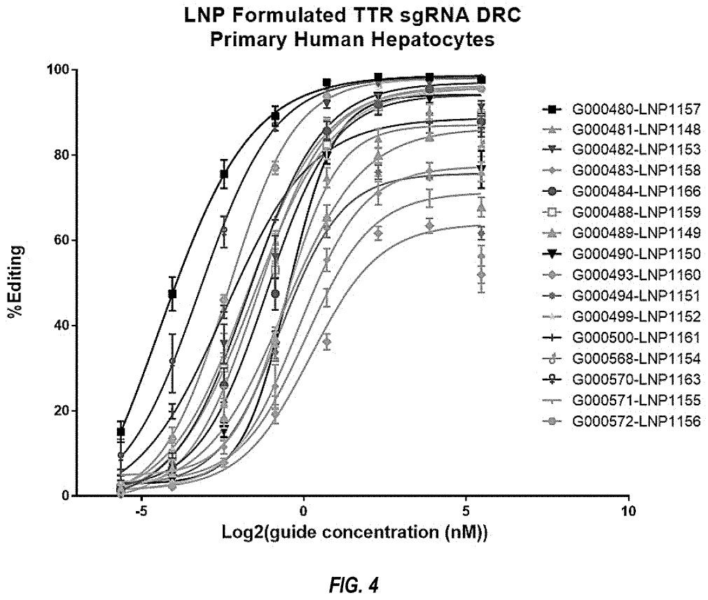

Lipid nanoparticle formulations of modified sgRNAs targeting human TTR and the cyno matched sgRNA sequences were tested on primary human hepatocytes and primary cyno hepatocytes in a dose response curve. Primary human and cyno hepatocytes were plated as described in Example 1. Both cell lines were incubated at 37° C., 5% CO₂ for 24 hours prior to treatment with LNPs. The LNPs used in the experiments detailed in Tables 18-21 were prepared using the Nanoassemblr™ procedure, each containing the specified sgRNA and Cas9 mRNA (SEQ ID NO:2), each having Lipid. The LNPs contained Lipid A, Cholesterol, DSPC, and PEG2k-DMG in a 45:44:9:2 molar ratio, respectively, and had a N:P ratio of 4.5. LNPs were incubated in hepatocyte maintenance media containing 6% cyno serum at 37° C. for 5 minutes. Post incubation the LNPs were added onto the primary human or cyno hepatocytes in an 8 point 2-fold dose response curve starting at 100 ng mRNA. The cells were lysed 72 hours post treatment for NGS analysis as described in Example 1. Percent editing was determined for crRNAs comprising each guide sequence across each cell type and the guide sequences were then rank ordered based on highest % editing at 12.5 ng mRNA input and 3.9 nM guide concentration. The dose response curve data for the guide sequences in both cell lines is shown in FIGS. 4 through 7. The % editing at 12.5 ng mRNA input and 3.9 nM guide concentration are listed below (Table 16 through 18).

Table 18 shows the average and standard deviation at 12.5 ng of cas9 mRNA for % Edit, % Insertion (Ins), and % Deletion (Del) for the tested TTR sgRNAs formulated in lipid nanoparticles with Spy Cas9 mRNA on primary human hepatocytes as dose response curves. G000570 exhibited an uncharacteristic dose response curve compared to the other sgRNAs which may be an artifact of the experiment. The data are shown graphically in FIG. 4.

TABLE 18
TTR editing data in primary human hepatocytes treated with
LNP formulated Spy Cas9 mRNA (SEQ ID NO: 2) and sgRNAs

| | 12.5 ng mRNA, | Std |
|---|---|---|
| | 3.9 nM sgRNA, | Dev % |
| GUIDE ID | Avg % Edit | Edit |
| G000480 | 59.33 | 0.73 |
| G000481 | 24.37 | 0.37 |
| G000482 | 19.10 | 2.64 |
| G000483 | 7.37 | 0.67 |
| G000484 | 16.67 | 1.23 |
| G000485 | 14.23 | 2.36 |
| G000486 | 61.33 | 2.59 |
| G000487 | 17.37 | 0.95 |
| G000488 | 44.80 | 3.00 |
| G000489 | 16.85 | 0.06 |
| G000490 | 10.53 | 1.90 |
| G000491 | 31.60 | 2.33 |
| G000492 | 15.87 | 0.44 |
| G000493 | 7.33 | 0.73 |
| G000494 | 6.37 | 1.07 |
| G000495 | 23.97 | 1.66 |
| G000496 | 30.73 | 3.76 |
| G000497 | 15.10 | 3.30 |
| G000498 | 24.43 | 1.30 |
| G000499 | 16.07 | 1.67 |
| G000500 | 23.57 | 2.44 |
| G000501 | 32.30 | 2.49 |
| G000567 | 48.95 | 1.06 |
| G000568 | 54.60 | 3.68 |
| G000570 | 88.30 | 1.84 |
| G000572 | 55.45 | 1.20 |

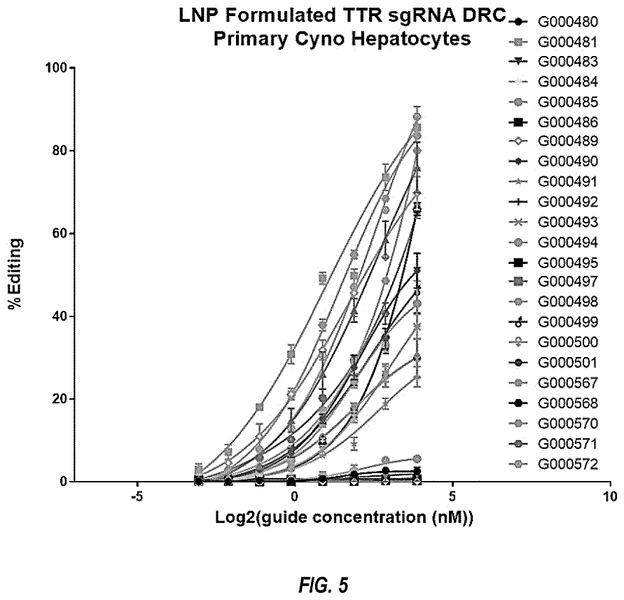

Table 19 shows the average and standard deviation at 12.5 ng of mNRA and 3.9 nM guide concentration for % Edit, % Insertion (Ins), and % Deletion (Del) for the tested TTR sgRNAs formulated in lipid nanoparticles with Spy Cas9 mRNA on primary cyno hepatocytes as dose response curves. The data are shown graphically in FIG. 5.

TABLE 19
TTR editing data in primary cyno hepatocytes treated with
LNP formulated Spy Cas9 mRNA (SEQ ID NO: 2) and sgRNAs

| | 12.5 ng mRNA, | Std |
|---|---|---|
| | 3.9 nM sgRNA, | Dev % |
| GUIDE ID | Avg % Edit | Edit |
| G000480 | 0.73 | 0.15 |
| G000481 | 49.20 | 1.39 |
| G000482 | 26.13 | 5.33 |
| G000483 | 0.73 | 0.60 |
| G000484 | 0.10 | 0.00 |
| G000485 | 1.43 | 1.02 |
| G000489 | 31.87 | 2.40 |
| G000490 | 15.23 | 1.08 |
| G000491 | 6.37 | 0.38 |
| G000492 | 0.70 | 0.28 |
| G000493 | 7.63 | 1.14 |
| G000494 | 14.30 | 1.06 |
| G000495 | 0.73 | 0.06 |
| G000497 | 0.23 | 0.06 |
| G000498 | 37.90 | 1.42 |
| G000499 | 14.63 | 0.70 |
| G000500 | 10.47 | 0.32 |
| G000501 | 1.37 | 0.31 |
| G000567 | 0.10 | 0.00 |
| G000568 | 9.25 | 0.21 |
| G000570 | 17.30 | 0.85 |
| G000571 | 20.20 | 2.26 |
| G000572 | 30.60 | 0.42 |

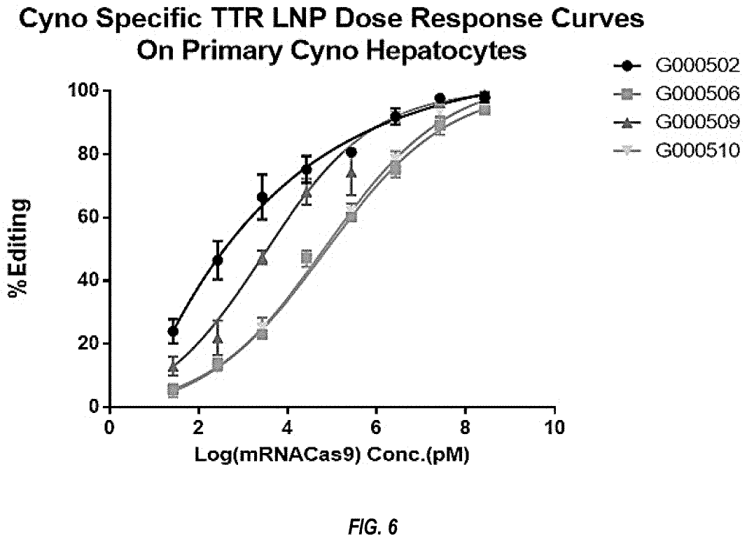

Table 20 shows the average and standard deviation at 12.5 ng of mRNA and 3.9 nM guide concentration for % Edit, % Insertion (Ins), and % Deletion (Del) for the tested cyno specific TTR sgRNAs formulated in lipid nanoparticles with Spy Cas9 mRNA on primary cyno hepatocytes as dose response curves. The data are shown graphically in FIG. 6.

TABLE 20
TTR editing data in primary cyno hepatocytes
treated with LNP formulated Spy Cas9 mRNA
(SEQ ID NO: 2) and cyno matched sgRNAs

| | 12.5 ng mRNA, | Std |
|---|---|---|
| | 3.9 nM sgRNA, | Dev % |
| GUIDE ID | Avg % Edit | Edit |
| G000502 | 80.70 | 0.14 |
| G000506 | 60.13 | 0.70 |
| G000509 | 74.47 | 7.28 |
| G000510 | 61.87 | 2.54 |

Cross Screening of LNP Formulated TTR sgRNAs with Spy Cas9 mRNA in Primary Human Hepatocytes and Primary Cyno Hepatocytes

Lipid nanoparticle formulations of modified sgRNAs targeting human TTR and the cyno matched sgRNA sequences were tested on primary human hepatocytes and primary cyno hepatocytes in a dose response curve. Primary human and cyno hepatocytes were plated as described in Example 1. Both cell lines were incubated at 37° C., 5% CO₂ for 24 hours prior to treatment with LNPs. The LNPs used in the experiments detailed in Tables 20-22 were prepared using the cross-flow procedure described above but purified using PD-10 columns (GE Healthcare Life Sciences) and concentrated using Amicon centrifugal filter units (Millipore Sigma), each containing the specified sgRNA and Cas9 mRNA (SEQ ID NO:1). The LNPs contained Lipid A, Cholesterol, DSPC, and PEG2k-DMG in a 50:38:9:3 molar ratio, respectively, and had a N:P ratio of 6.0. LNPs were incubated in hepatocyte maintenance media containing 6% cyno serum at 37° C., 5% CO₂ for 5 minutes. Post incubation the LNPs were added onto the primary human or cyno hepatocytes in an 8 point 3-fold dose response curve starting at 300 ng mRNA. The cells were lysed 72 hours post treatment for NGS analysis as described in Example 1. Percent editing was determined for crRNAs comprising each guide sequence across each cell type and the guide sequences were then rank ordered based on EC50 values and maximum editing percent. The dose response curve data for the guide sequences in both cell lines is shown in FIGS. 4 through 7. The EC 50 values and maximum editing percent are listed below (Table 19 through 22).

Table 21 shows the EC50 and maximum editing the tested human specific TTR sgRNAs formulated in lipid nanoparticles with U-depleted Spy Cas9 mRNA on primary human hepatocytes as dose response curves. The data are shown graphically in FIG. 4.

TABLE 21
TTR editing data in primary human hepatocytes treated with
LNP formulated Spy Cas9 mRNA and human specific sgRNAs

| GUIDE ID | EC50 | Max Editing |
|---|---|---|
| G000480 | 0.10 | 98.69 |
| G000481 | 1.43 | 87.05 |
| G000482 | 0.65 | 97.02 |
| G000483 | 1.88 | 77.39 |
| G000484 | 0.95 | 94.14 |
| G000488 | 0.72 | 95.83 |
| G000489 | 1.38 | 86.33 |
| G000490 | 1.52 | 94.16 |
| G000493 | 2.42 | 63.95 |
| G000494 | 1.28 | 75.70 |
| G000499 | 0.63 | 96.31 |
| G000500 | 0.39 | 88.70 |
| G000568 | 0.78 | 95.72 |
| G000570 | 0.23 | 98.22 |
| G000571 | 2.21 | 71.28 |
| G000572 | 0.42 | 97.94 |

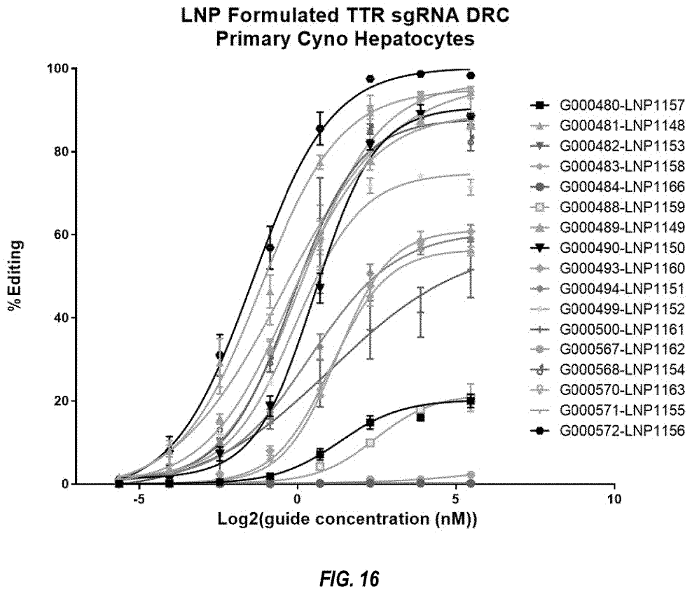

Table 22 shows the EC50 and maximum editing the tested human specific TTR sgRNAs formulated in lipid nanoparticles with U-depleted Spy Cas9 mRNA on primary cyno hepatocytes as dose response curves. The data are shown graphically in FIG. 16.

TABLE 22
TTR editing data in primary cyno hepatocytes treated with
LNP formulated Spy Cas9 mRNA and human specific sgRNAs

| GUIDE ID | EC50 | Max Editing |
|---|---|---|
| G000480 | 5.28 | 20.32 |
| G000481 | 0.93 | 95.07 |
| G000482 | 0.89 | 97.47 |
| G000483 | 4.40 | 56.52 |
| G000484 | 3.47 | 0.22 |
| G000488 | 11.56 | 21.63 |
| G000489 | 1.79 | 89.21 |
| G000490 | 3.09 | 90.76 |
| G000493 | 4.97 | 61.15 |
| G000494 | 2.77 | 60.84 |
| G000499 | 2.00 | 74.94 |
| G000500 | 4.42 | 58.04 |
| G000567 | 1.76 | 97.06 |
| G000568 | 1.87 | 87.93 |
| G000570 | 2.00 | 96.73 |
| G000571 | 1.55 | 97.03 |
| G000572 | 0.79 | 100.31 |
| G000504 | 5.16 | 7.16 |
| G000505 | 3.57 | 13.48 |
| G000506 | 1.26 | 89.49 |

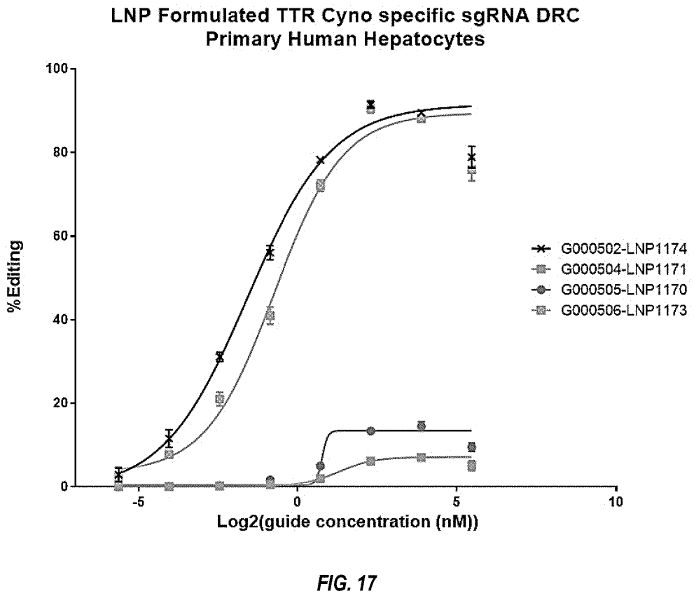

Table 23 shows the EC50 and maximum editing the tested cyno matched TTR sgRNAs formulated in lipid nanoparticles with U-depleted Spy Cas9 mRNA on primary human hepatocytes as dose response curves. The data are shown graphically in FIG. 17.

TABLE 23
TTR editing data in primary human hepatocytes treated with
LNP formulated Spy Cas9 mRNA and cyno specific sgRNAs

| GUIDE ID | EC50 | Max Editing |
|---|---|---|
| G000502 | 0.70 | 91.5 |
| G000504 | 5.16 | 7.16 |
| G000505 | 3.57 | 13.48 |
| G000506 | 1.26 | 89.49 |

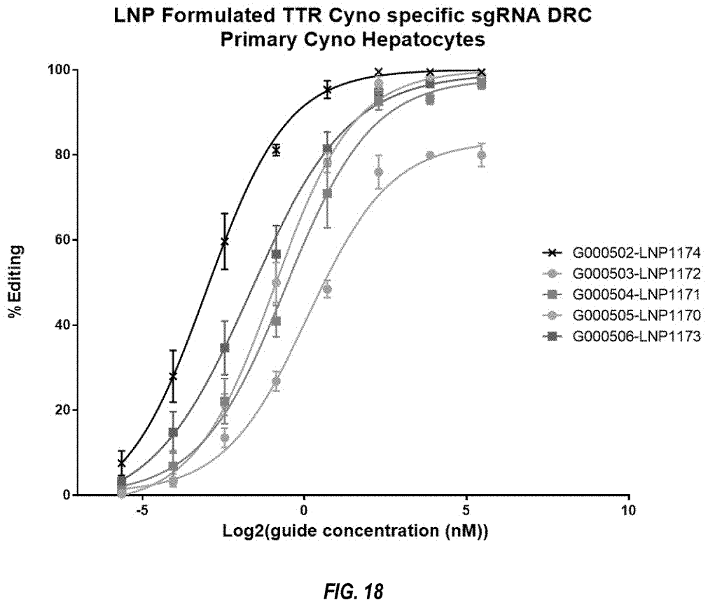

Table 24 shows the EC50 and maximum editing the tested cyno matched TTR sgRNAs formulated in lipid nanoparticles with U-depleted Spy Cas9 mRNA on primary cyno hepatocytes as dose response curves. The data are shown graphically in FIG. 18.

TABLE 24
TTR editing data in primary cyno hepatocytes treated with
LNP formulated Spy Cas9 mRNA and cyno specific sgRNAs

| GUIDE ID | EC50 | Max Editing |
|---|---|---|
| G000502 | 0.26 | 100.05 |
| G000503 | 2.26 | 83.41 |
| G000504 | 1.42 | 98.04 |
| G000505 | 1.10 | 99.97 |
| G000506 | 0.66 | 99.18 |

### Example 5. Off-Target Analysis of TTR dgRNAs and sgRNAs

### Off-Target Analysis of TTR Guides

An oligo insertion based assay (See, e.g., Tsai et al., Nature Biotechnology 33, 187-197; 2015) was used to determine potential off-target genomic sites cleaved by Cas9 targeting TTR. Forty-five dgRNAs from Table 1 (and two control guides with known off-target profiles) were screened in the HEK293_Cas9 cells. The human embryonic kidney adenocarcinoma cell line HEK293 constitutively expressing Spy Cas9 (“HEK293_Cas9”) was cultured in DMEM media supplemented with 10% fetal bovine serum and 500 μg/ml G418. Cells were plated at a density of 30,000 cells/well in a 96-well plate 24 hours prior to transfection. Cells were transfected with Lipofectamine RNAiMAX (ThermoFisher, Cat. 13778150) per the manufacturer's protocol. Cells were transfected with a lipoplex containing individual crRNA (15 nM), trRNA (15 nM), and donor oligo with (10 nM) Lipofectamine RNAiMAX (0.3 μL/well) and OptiMem. Cells were lysed 24 hours post transfection and genomic DNA was extracting using Zymo's Quick gDNA 96 Extraction kit (catalog #D3012) following the manufacturer's recommended protocol. The gDNA was quantified using the Qubit High Sensitivity dsDNA kit (Life Technologies). Libraries were prepared per the previously described method in Tsai et al, 2015 with minor modifications. Sequencing was performed on Illumina's MiSeq and HiSeq 2500. The assay identified potential off-target sites for some of the crRNAs which are plotted in FIG. 2.

Table 25 shows the number of off-target integration sites detected in HekCas9 cells transfected with TTR dgRNAs along with a double stranded DNA oligo donor sequence.

TABLE 25
Number of off-target integration sites detected
for TTR dgRNAs via an oligo insertion based assay

| GUIDE ID | # Sites |
|---|---|
| CR003335 | 0 |
| CR003336 | 2 |
| CR003337 | 10 |
| CR003338 | 2 |
| CR003339 | 3 |
| CR003340 | 0 |
| CR003342 | 0 |
| CR003343 | 2 |
| CR003344 | 0 |
| CR003345 | 0 |
| CR003346 | 0 |
| CR003347 | 1 |
| CR003348 | 3 |
| CR003351 | 1 |
| CR003352 | 2 |
| CR003353 | 2 |
| CR003355 | 1 |
| CR003356 | 4 |
| CR003357 | 3 |
| CR003359 | 6 |
| CR003360 | 0 |
| CR003363 | 4 |
| CR003365 | 3 |
| CR003366 | 1 |
| CR003367 | 1 |
| CR003368 | 2 |
| CR003369 | 2 |
| CR003377 | 0 |
| CR003380 | 0 |
| CR003382 | 34 |
| CR003383 | 1 |
| CR003385 | 3 |
| CR003386 | 1 |
| CR003387 | 6 |
| CR003388 | 2 |
| CR003389 | 2 |
| CR003390 | 1 |
| CR003391 | 0 |
| CR003392 | 0 |
| CR005298 | 0 |
| CR005300 | 0 |
| CR005301 | 0 |
| CR005302 | 1 |
| CR005303 | 1 |
| CR005304 | 0 |

Additionally, a subset of the guides was assessed for off-target potential as modified sgRNAs in the Hek_Cas9 cells via the oligo based insertion method described above. The off-target results were plotted in FIG. 4.

Table 26 shows the number of off-target integration sites detected in HekCas9 cells transfected with TTR sgRNAs along with a double stranded DNA oligo donor sequence.

TABLE 26
Number of off-target integration sites detected
for TTR sgRNAs via an insertion detection method

| GUIDE ID | # Sites |
|---|---|
| G000480 | 11 |
| G000481 | 3 |
| G000482 | 13 |
| G000483 | 5 |
| G000484 | 7 |
| G000485 | 22 |
| G000486 | 12 |
| G000487 | 14 |
| G000488 | 0 |
| G000489 | 19 |
| G000490 | 12 |
| G000491 | 28 |
| G000492 | 97 |
| G000493 | 7 |
| G000494 | 4 |
| G000495 | 13 |
| G000496 | 1 |
| G000497 | 26 |
| G000498 | 82 |
| G000499 | 4 |
| G000500 | 46 |
| G000501 | 4 |
| G000567 | 9 |
| G000568 | 937 |
| G000570 | 19 |
| G000571 | 16 |
| G000572 | 15 |

### Example 6. Targeted Sequencing for Validating Potential Off-Target Sites

The HEK293_Cas9 cells used in Example 5 for detecting potential off-targets constitutively overexpress Cas9, leading to a higher number of potential off-target “hits” as compared to a transient delivery paradigm in various cell types. Further, when delivering sgRNAs (as opposed to dgRNAs), the number of potential off-target hits may be further inflated as sgRNA molecules are more stable than dgRNAs (especially when chemically modified). Accordingly, potential off-target sites identified by an oligo insertion method as used in Example 5 may be validated using targeted sequencing of the identified potential off-target sites.

In one approach, primary hepatocytes are treated with LNPs comprising Cas9 mRNA and a sgRNA of interest (e.g., a sgRNA having potential off-target sites for evaluation). The primary hepatocytes are then lysed and primers flanking the potential off-target site(s) are used to generate an amplicon for NGS analysis. Identification of indels at a certain level may validate potential off-target site, whereas the lack of indels found at the potential off-target site may indicate a false positive in the HEK293_Cas9 cell assay.

### Example 7. Phenotypic Analysis

### Western Blot Analysis of Secreted TTR

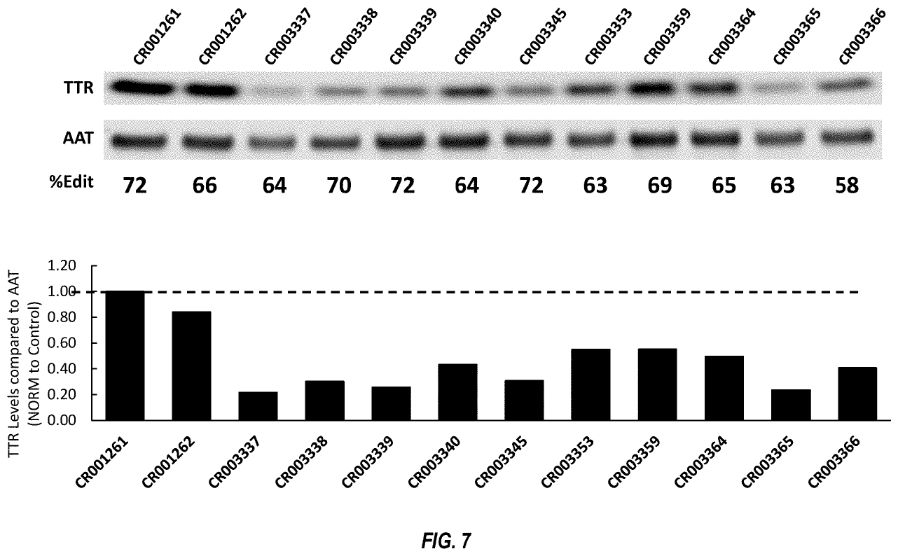

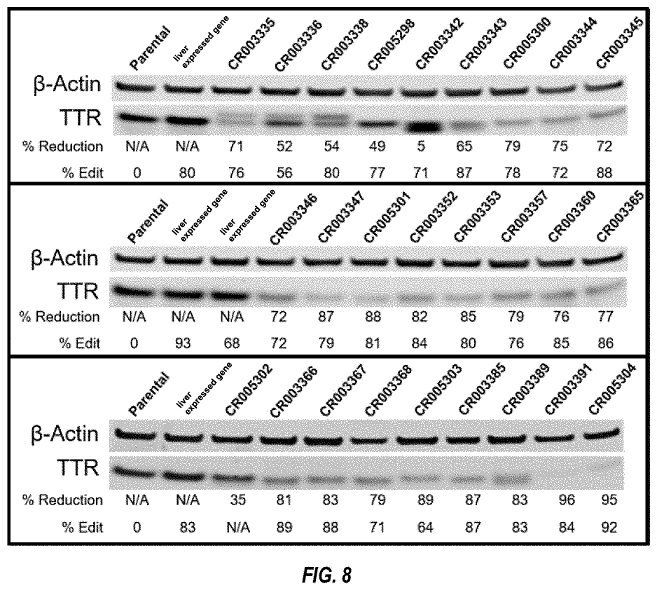

The hepatocellular carcinoma cell line, HepG2, was transfected as described in Example 1 with select guides from Table 1 in triplicate. Two days post-transfection, one replicate was harvested for genomic DNA and analysis by NGS sequencing for editing efficiency. Five days post-transfection, media without serum was replaced on one replicate. After 4 hrs the media was harvested for analysis of secreted TTR by WB as previously described. The data for % edit for each guide and reduction of extracellular TTR is provided in FIG. 7.

### Western Blot Analysis of Intracellular TTR

The hepatocellular carcinoma cell line, HUH7, was transfected as described in Example 1 with crRNA comprising the guides from Table 1. The transfected pools of cells were retained in tissue culture and passaged for further analysis. At seven days post-transfection, cells were harvested and whole cell extracts (WCEs) were prepared and subjected to analysis by Western Blot as previously described.

WCEs were analyzed by Western Blot for reduction of TTR protein. Full length R protein has a predicted molecular weight of ˜16 kD. A band at this molecular weight was observed in the control lanes in the Western Blot.

Percent reduction of TTR protein was calculated using the Licor Odyssey Image Studio Ver 5.2 software. GAPDH was used as a loading control and probed simultaneously with TTR. A ratio was calculated for the densitometry values for GAPDH within each sample compared to the total region encompassing the TTR band. Percent reduction of TTR protein was determined after the ratios were normalized to control lanes. Results are shown in FIG. 8.

### Example 8. LNP Delivery to Humanized TTR Mice and Mice Having Wt (Murine) TTR

Mice humanized with respect to the TTR gene were dosed with LNP formulations 701-704 containing the guides indicated in Table 27 (5 mice per formulation). These humanized TTR mice were engineered such that a region of the endogenous murine TTR locus was deleted and replaced with an orthologous human TTR sequence so that the locus encodes a human TTR protein. For comparison, 6 mice with murine TTR were dosed with LNP700, containing a guide (G000282) targeting murine TTR. LNPs with Formulation Numbers 1-5 in Table 27 were prepared using the Nanoassemblr™ procedure as described above while LNPs with Formulation Numbers 6-16 were prepared using the cross-flow procedure described above but purified using PD-10 columns (GE Healthcare Life Sciences) and concentrated using Amicon centrifugal filter units (Millipore Sigma). As negative controls, mice of the corresponding genotype were dosed with vehicle alone (Tris-saline-sucrose buffer (TSS)). The background of the humanized TTR mice administered LNPs with Formulation Numbers 2-5 in Table 27 was 50% 12956/SvEvTac 50% C57BL/6NTac; the background of the humanized TTR mice administered LNPs having Formulation Numbers 6-16 in Table 25 as well as the mice with murine TTR (administered LNP700, Formulation Number 1) was 75% C57BL/6NTac 25% 12956/SvEvTac.

TABLE 27
LNP formulations for dosing humanized TTR mice.

| | | | | | Molar Ratios |
|---|---|---|---|---|---|
| | | | | | (Lipid A, |
| | | | RNA | | Cholesterol, |
| | | | concen- | | DSPC, and |
| Formulation | | | tration | N:P | PEG2k-DMG, |
| Number | LNP | Guide | (mg/ml) | Ratio | respectively) |
| 1 | LNP700 | G000282 | 0.53 | 4.5 | 45:44:9:2 |
| 2 | LNP701 | G000481 | 0.46 | 4.5 | 45:44:9:2 |
| 3 | LNP702 | G000489 | 0.61 | 4.5 | 45:44:9:2 |
| 4 | LNP703 | G000494 | 0.57 | 4.5 | 45:44:9:2 |
| 5 | LNP704 | G000499 | 0.59 | 4.5 | 45:44:9:2 |
| 6 | LNP1148 | G000481 | 0.73 | 4.5 | 45:44:9:2 |
| 7 | LNP1152 | G000499 | 0.45 | 6.0 | 50:38:9:3 |
| 8 | LNP1153 | G000482 | 0.53 | 6.0 | 50:38:9:3 |
| 9 | LNP1155 | G000571 | 0.70 | 6.0 | 50:38:9:3 |
| 10 | LNP1156 | G000572 | 0.58 | 6.0 | 50:38:9:3 |
| 11 | LNP1157 | G000480 | 0.84 | 6.0 | 50:38:9:3 |
| 12 | LNP1159 | G000488 | 0.79 | 6.0 | 50:38:9:3 |
| 13 | LNP1160 | G000493 | 0.71 | 6.0 | 50:38:9:3 |
| 14 | LNP1161 | G000500 | 0.66 | 6.0 | 50:38:9:3 |
| 15 | LNP1162 | G000567 | 0.69 | 6.0 | 50:38:9:3 |
| 16 | LNP1163 | G000570 | 0.66 | 6.0 | 50:38:9:3 |

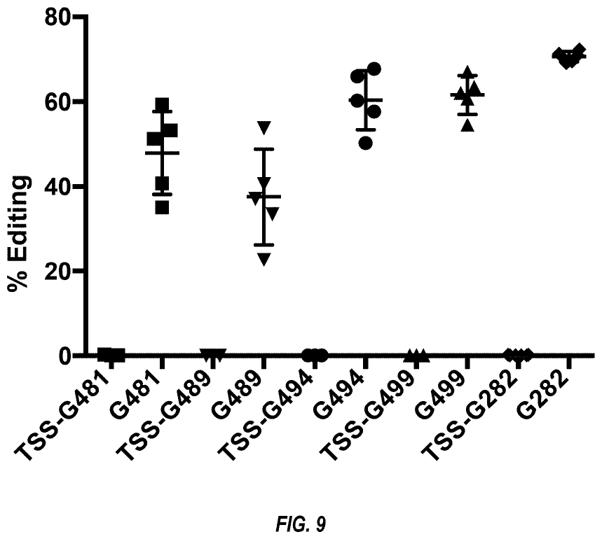

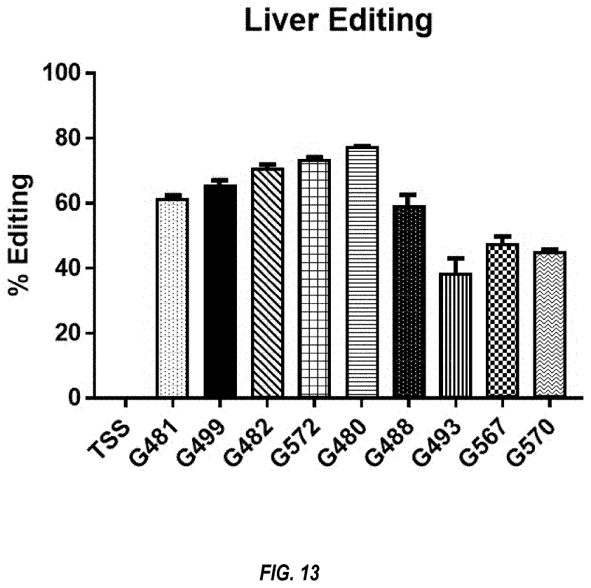

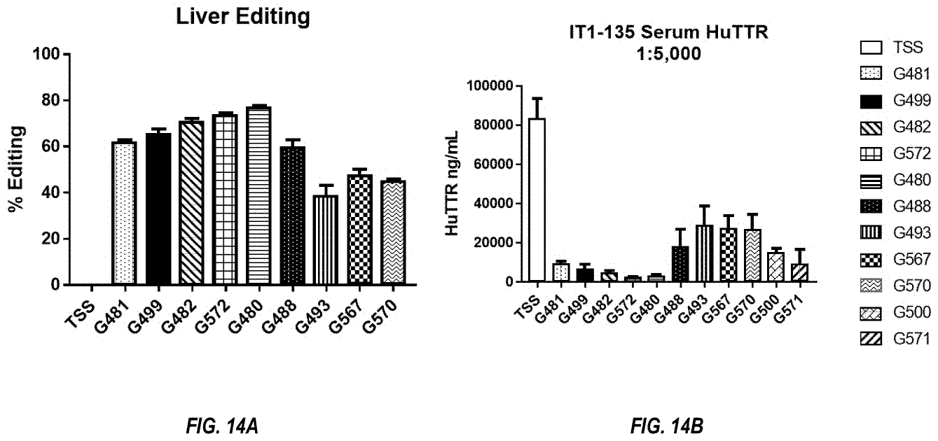

LNPs having Formulation numbers 1-5 contained Cas9 mRNA of SEQ ID NO:2 and LNPs having Formulation Numbers 6-16 contained Cas9 mRNA of SEQ ID NO: 1, all in a 1:1 ratio by weight to the guide. The LNPs contained Lipid A, Cholesterol, DSPC, and PEG2k-DMG in the molar ratios recited in Table 27, respectively. Dosing with LNPs having Formulation Numbers 1-5 was at 2 mg/kg (total RNA content) and dosing with LNPs having Formulation Numbers 6-16 was at 1 mg/kg (total RNA content). Liver editing results were determined using primers designed to amplify the region of interest for NGS analysis. Liver editing results for Formulation Numbers 1-5 are shown in FIG. 9 and indicate editing of the human TTR sequence with each of the four guides tested at a level >35% editing (mean values) with G000494 and G000499 providing values near 60%. Liver editing results for formulation numbers 6-8, 10-13, and 15-16 are shown in FIG. 13 and Table 28, which show efficient editing of the human TTR sequence with each of the formulations tested. Greater than 38% editing was seen for all formulations, with several formulations providing editing values greater than 60%. Formulations 9 and 14 are not shown due to the design of the PCR amplicon and a resulting low number of sequencing reads.

The level of human TTR in serum was measured in the mice provided formulation numbers 6-8, 10-13, and 15-16. See FIG. 14B. FIG. 14A is a repeat of FIG. 13 provided for comparison purposes. Knockdown of serum human TTR was detected for each formulation tested, which correlated with the amount of editing detected in liver (See FIG. 14A vs 14B, Table 28).

TABLE 28

| GUIDE ID | % Editing | Serum TTR(% TSS) |
|---|---|---|
| TSS (vehicle) | 0.06 | 100 |
| G481 | 61.28 | 10.52 |
| G499 | 65.66 | 8.39 |
| G482 | 70.86 | 4.65 |
| G572 | 73.52 | 2.11 |
| G480 | 77.34 | 3.48 |
| G488 | 59.125 | 27.78 |
| G493 | 38.55 | 49.73 |
| G567 | 47.525 | 44.24 |
| G570 | 45.5 | 41.73 |
| G571 | 33.88 | 11.39 |
| G500 | 44.44 | 34.28 |

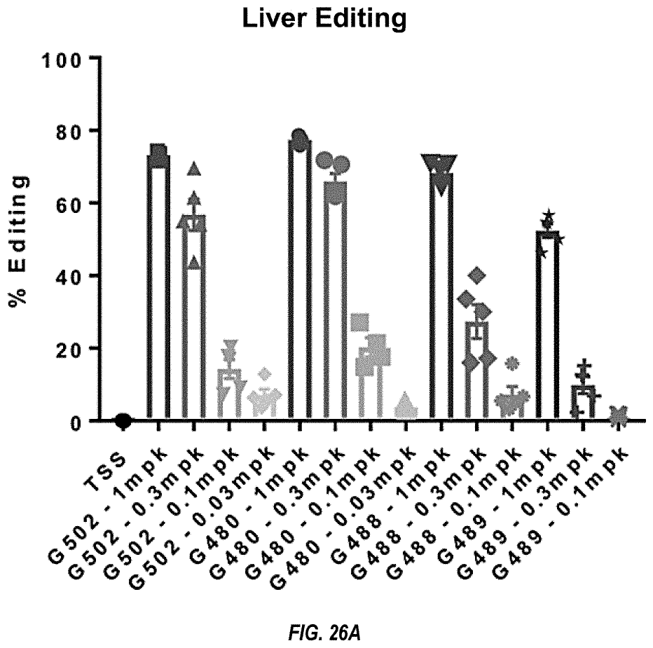

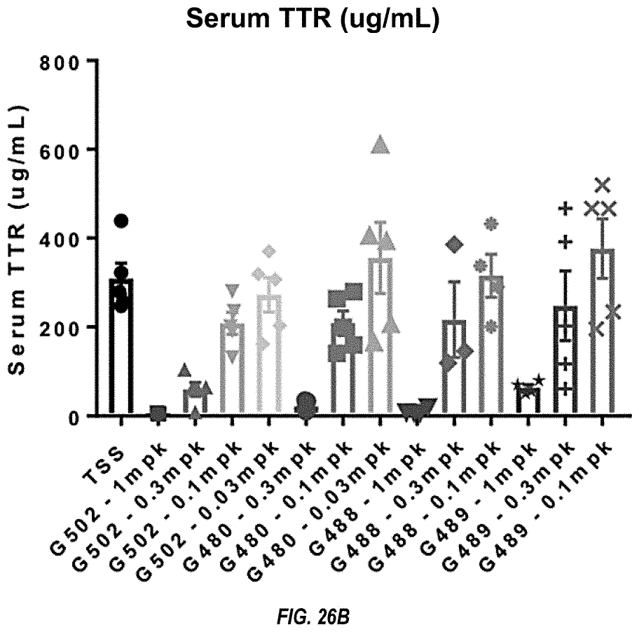

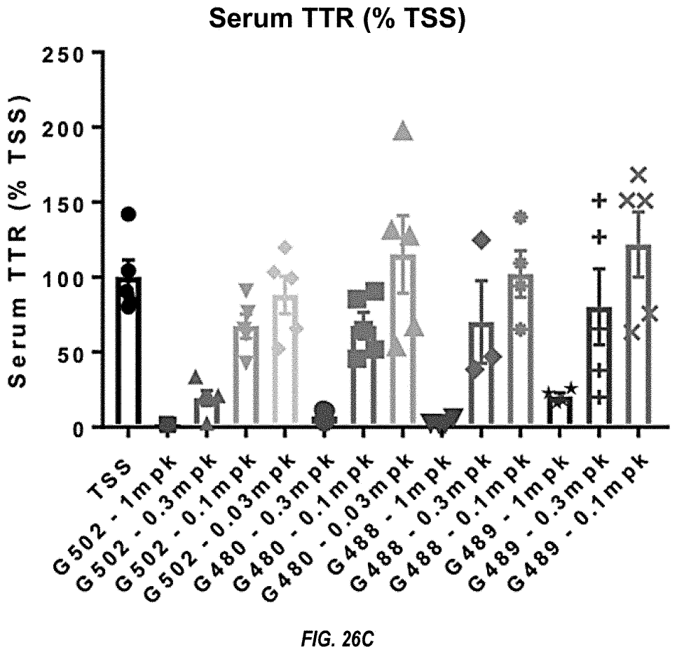

In another set of experiments, humanized TTR mice were dosed with LNP formulations across a range of doses with guides G000480, G000488, G000489 and G000502. The formulations contained Cas9 mRNA (SEQ ID NO: 1) in a 1:1 ratio by weight to the guide. The LNPs contained Lipid A, Cholesterol, DSPC, and PEG2k-DMG in a 50:38:9:3 molar ratio, respectively, and having a N:P ratio of 6. Dosing was at 1, 0.3, 0.1, or 0.03 mg/kg (n=5/group). The LNPs were prepared using the cross-flow procedure described above and purified and concentrated using PD-10 columns and Amicon centrifugal filter units, respectively. Liver editing results were determined using primers designed to amplify the region of interest for NGS analysis and serum human TTR levels were measured as described above. Results for liver editing are shown in FIG. 26A and serum human TTR levels in FIG. 26B-C. A dose response for both editing and serum TTR levels was evident.

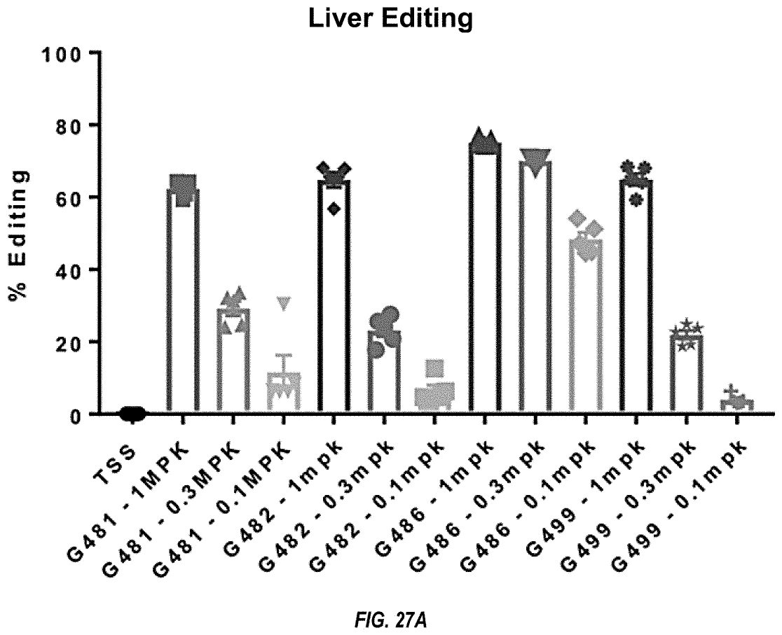

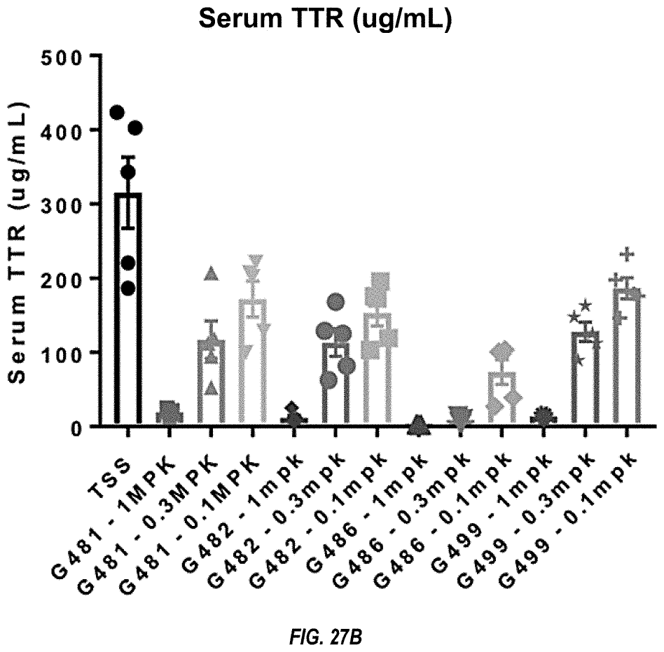

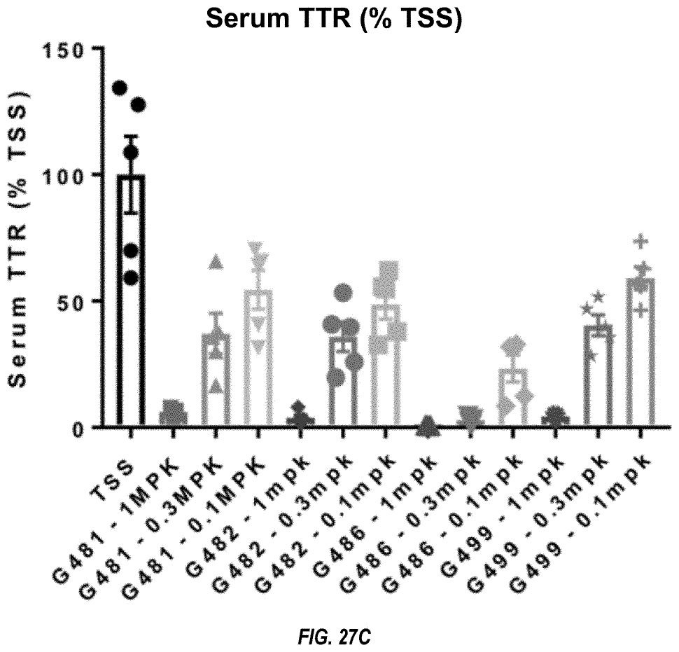

In another set of experiments, humanized TTR mice were dosed with LNP formulations across a range of doses with guides G000481, G000482, G000486 and G000499. The formulations contained Cas9 mRNA (SEQ ID NO: 1) in a 1:1 ratio by weight to the guide. The LNPs contained Lipid A, Cholesterol, DSPC, and PEG2k-DMG in a 50:38:9:3 molar ratio, respectively, and had an N:P ratio of 6. Dosing was at 1, 0.3, or 0.1 mg/kg (n=5/group). The LNPs were prepared using the cross-flow procedure described above and purified and concentrated using PD-10 columns and Amicon centrifugal filter units, respectively. Liver editing results were determined using primers designed to amplify the region of interest for NGS analysis and serum human TTR levels were measured as described above. Results for liver editing are shown in FIG. 27A and serum human TTR levels in FIG. 27B-C. A dose response for both editing and serum TTR levels was evident.

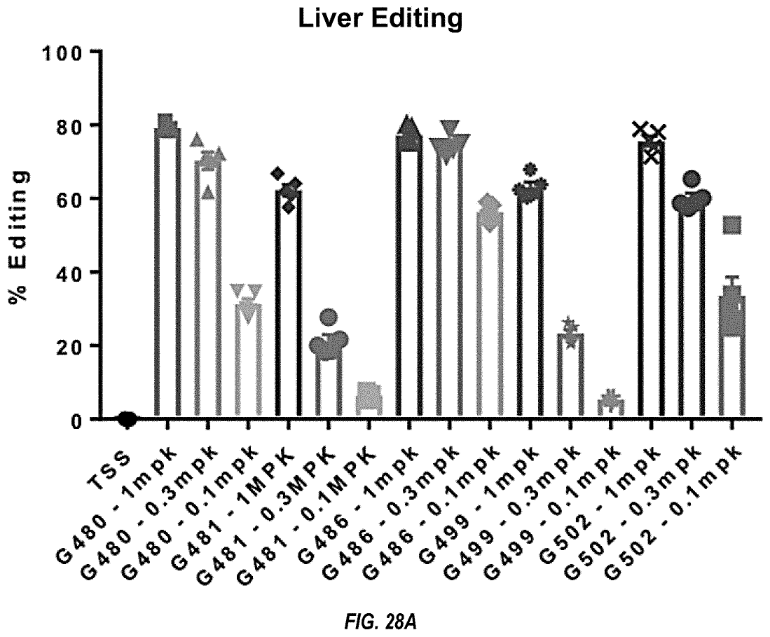

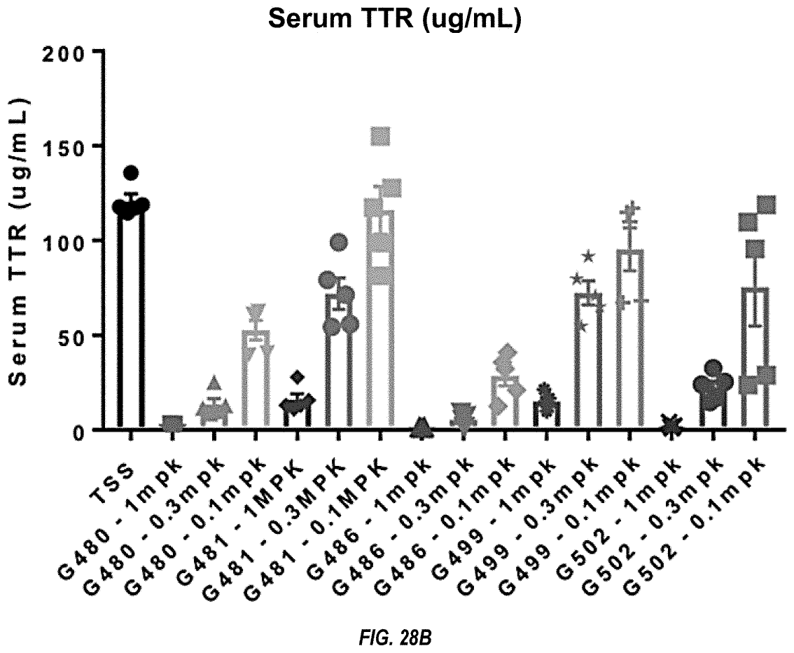

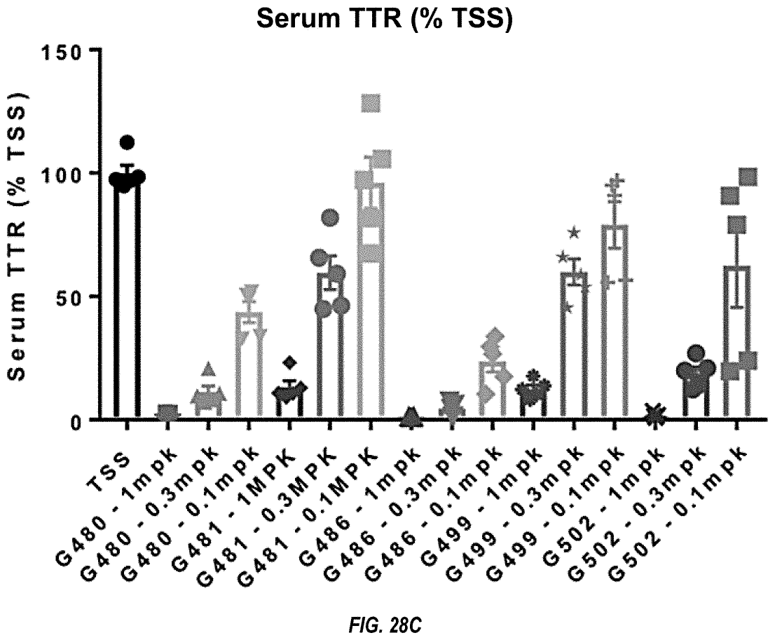

In another set of experiments, humanized TTR mice were dosed with LNP formulations across a range of doses with guides G000480, G000481, G000486, G000499 and G000502. The formulations contained Cas9 mRNA (SEQ ID NO: 1) in a 1:2 ratio by weight to the guide. The LNPs contained Lipid A, Cholesterol, DSPC, and PEG2k-DMG in a 50:38:9:3 molar ratio, respectively, and had an N:P ratio of 6. Dosing was at 1, 0.3, or 0.1 mg/kg (n=5/group). The LNPs were prepared using the cross-flow procedure described above and purified and concentrated using PD-10 columns and Amicon centrifugal filter units, respectively. Liver editing results were determined using primers designed to amplify the region of interest for NGS analysis and serum human TTR levels were measured as described above. Results for liver editing are shown in FIG. 28A and serum human TTR levels in FIG. 28B-C. A dose response for both editing and serum TTR levels was evident.

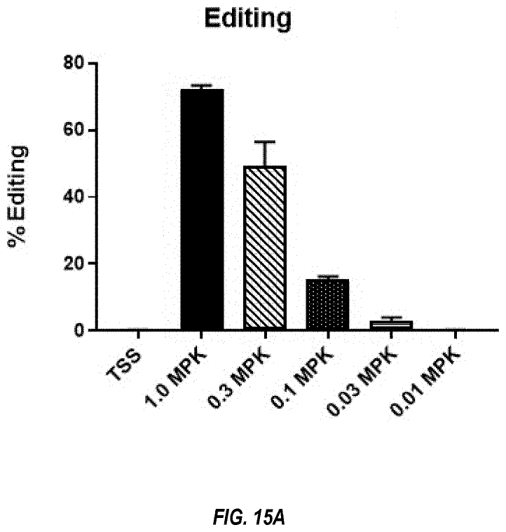

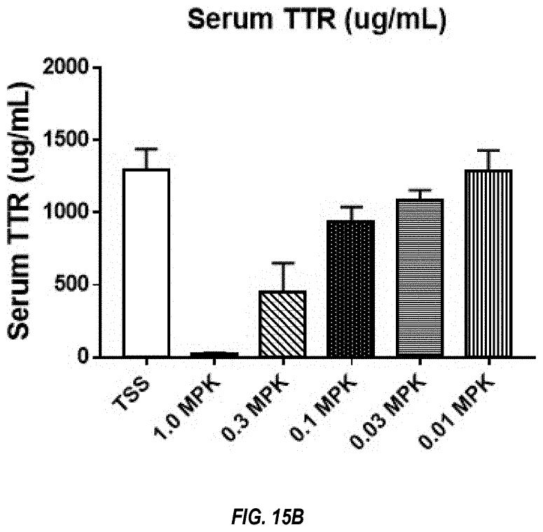

In separate experiments using wild type CD-1 mice, an LNP formulation comprising guide G000502, which is cross homologous between mouse and cyno, was tested in a dose response study. The formulation contained Cas9 mRNA (SEQ ID NO: 1) in a 1:1 ratio by weight to the guide. The LNP contained Lipid A, Cholesterol, DSPC, and PEG2k-DMG in a 45:44:9:2 molar ratio, respectively, and having a N:P ratio of 6. Dosing was at 1, 0.3, 0.1, 0.03, or 0.01 mg/kg (n=5/group). Liver editing results were determined using primers designed to amplify the region of interest for NGS analysis. Results for liver editing are shown in FIG. 15A and serum mouse TTR levels in FIG. 15B. A dose response for both editing and serum TTR levels was evident.

### Example 9. LNP Delivery to Mice in Multiple Doses

Mice (females from Charles River Laboratory, aged approximately 6-7 weeks) were dosed with an LNP formulation LNP705, prepared using cross-flow and TFF procedures as described above containing G000282 (“G282”) and Cas9 mRNA (SEQ ID NO: 2) in a 1:1 ratio by weight and a total RNA concentration of 0.5 mg/ml. The LNP had an N:P ratio of 4.5 and contained Lipid A, Cholesterol, DSPC, and PEG2k-DMG in a 45:44:9:2 molar ratio, respectively. Groups were dosed either once weekly up to one, two, three, or four weeks (QW×1-4) or once monthly up to two or three months (QM×2-3). Dosages were 0.5 mg/kg or 1 mg/kg (total RNA content). Control groups received a single dose on day 1 of 0.5, 1, or 2 mg/kg. Each group contained 5 mice. Serum TTR was analyzed by ELISA and at necropsy the liver, spleen and muscle were each collected for NGS editing analysis. Groups are shown in Table 29. X=sacrifice and necropsy. MPK=mg/kg.

TABLE 29
Study Groups

| | | | Total | | | | | | | |
|---|---|---|---|---|---|---|---|---|---|---|
| | Duration/ | | Dose | Dose | Dose | Dose | Dose | NX | Dose | NX |
| | Dose | Dose | (MPK) | Day | Day | Day | Day | Day | Day | Day |
| Group | Regimen | (MPK) | Given | 1 | 8 | 15 | 22 | 28 | 43 | 49 |
| 1 | 4 Week | 0 (TSS | 0 | X | X | X | X | X | | |
| | Multi Dose/ | control) | | | | | | | | |
| | QWx4 | | | | | | | | | |
| 2 | 2 Month | 1 | 3 | X | | | X | | X | X |
| 3 | Multi Dose/ | 0.5 | 1.5 | X | | | X | | X | X |
| | QMx3 | | | | | | | | | |
| 4 | 1 Month | 1 | 2 | X | | | X | X | | |
| 5 | Multi Dose/ | 0.5 | 1 | X | | | X | X | | |
| | QMx2 | | | | | | | | | |
| 6 | 4 Week | 1 | 4 | X | X | X | X | X | | |
| 7 | Multi Dose/ | 0.5 | 2 | X | X | X | X | X | | |
| | QWx4 | | | | | | | | | |
| 8 | 3 Week | 1 | 3 | | X | X | X | X | | |
| 9 | Multi Dose/ | 0.5 | 1.5 | | X | X | X | X | | |
| | QWx3 | | | | | | | | | |
| 10 | 2 Week | 1 | 2 | | | X | X | X | | |
| 11 | Multi Dose/ | 0.5 | 1 | | | X | X | X | | |
| | QWx2 | | | | | | | | | |
| 12 | Single Dose/ | 1 | 1 | | | | X | X | | |
| 13 | QWx1 | 0.5 | 0.5 | | | | X | X | | |
| 14 | | 2 | 2 | | | | Day | Day | | |
| | | | | | | | 26 | 32 | | |

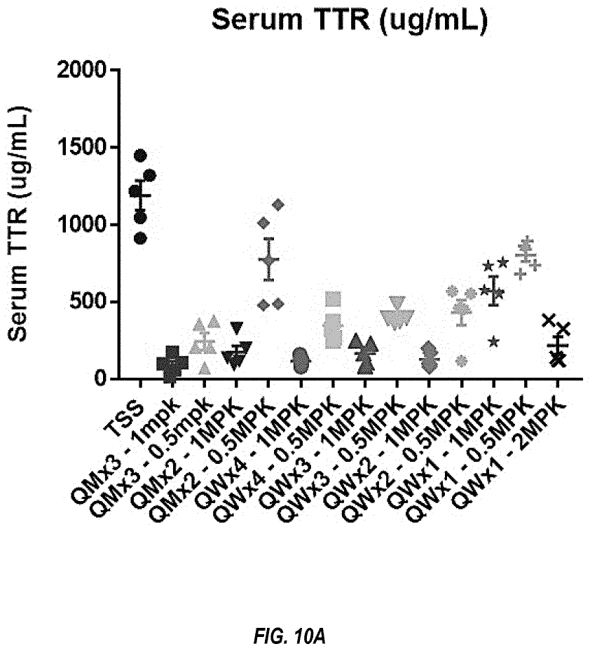

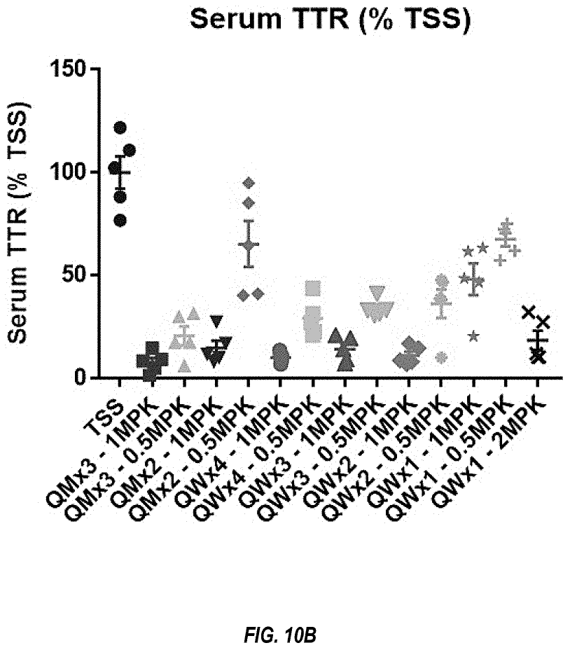

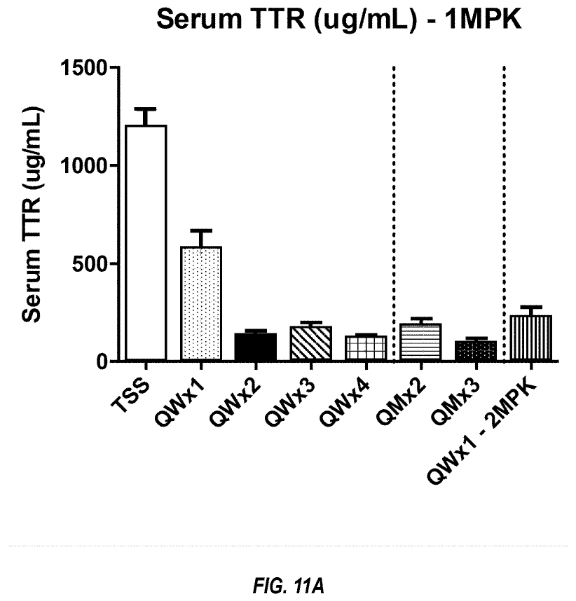

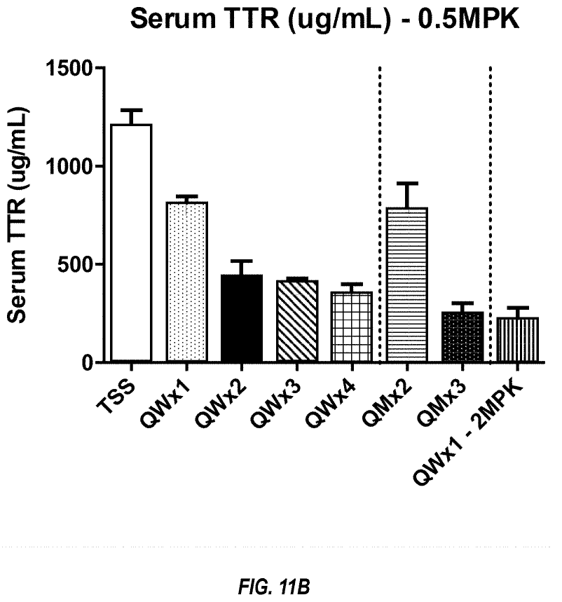

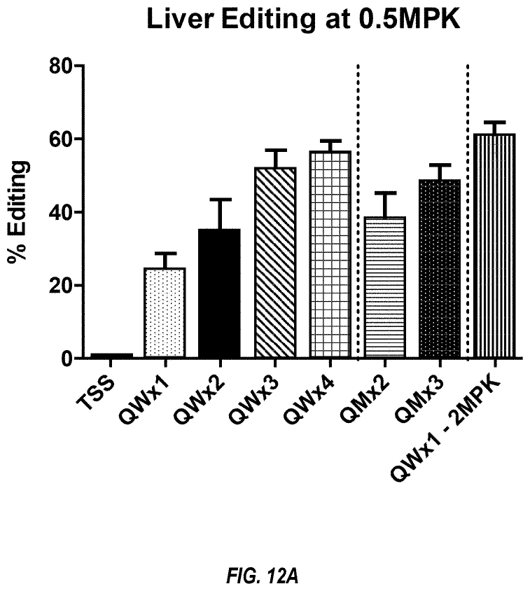

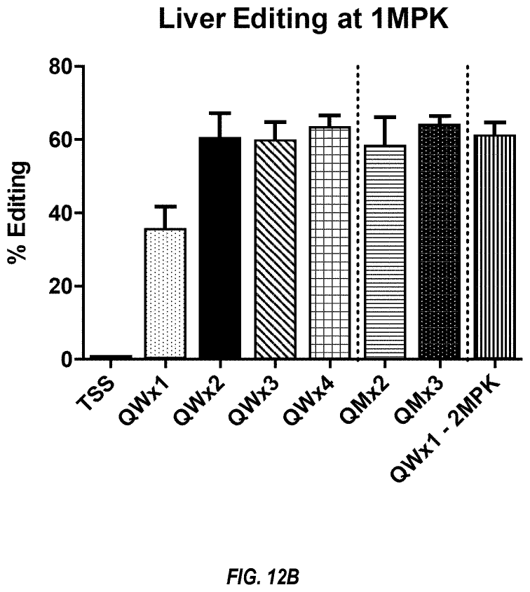

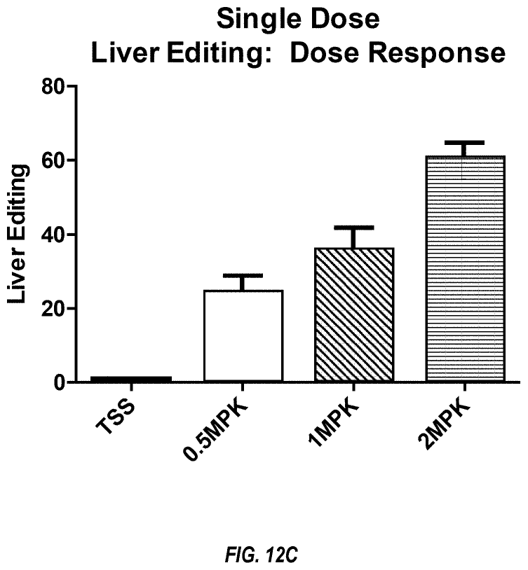

Table 30 and FIGS. 10A-11B show serum TTR level results (% KD=% knockdown). Table 30 and FIGS. 12A-C show liver editing results.

TABLE 30
Serum TTR Results.

| Time | | Serum TTR | Serum TTR |
|---|---|---|---|
| Regimen | Dose | (μg/mL) | (% KD) |
| QWx4 | TSS | 1190.7 | — |
| QMx3 | 0.5 | 245.01 | 79.42 |
| QMx2 | 0.5 | 776.73 | 34.77 |
| QWx4 | 0.5 | 347.43 | 70.82 |
| QWx3 | 0.5 | 405.70 | 65.93 |
| QWx2 | 0.5 | 432.25 | 63.70 |
| QWx1 | 0.5 | 804.06 | 32.47 |
| QMx3 | 1 | 91.95 | 92.28 |
| QMx2 | 1 | 176.81 | 85.15 |
| QWx4 | 1 | 119.52 | 89.96 |
| QWx3 | 1 | 167.15 | 85.96 |
| QWx2 | 1 | 130.98 | 89.00 |
| QWx1 | 1 | 573.02 | 51.88 |
| QWx1 | 2 | 219.07 | 81.60 |

TABLE 31
Liver Editing Results.

| Time | | Liver Editing |
|---|---|---|
| Regimen | Dose | (%) |
| QWx4 | TSS | 0.38 |
| QMx3 | 0.5 | 48.18 |
| QMx2 | 0.5 | 36.66 |
| QWx4 | 0.5 | 56.03 |
| QWx3 | 0.5 | 51.35 |
| QWx2 | 0.5 | 34.77 |
| QWx1 | 0.5 | 24.16 |
| QMx3 | 1 | 63.40 |
| QMx2 | 1 | 57.37 |
| QWx4 | 1 | 62.89 |
| QWx3 | 1 | 59.22 |
| QWx2 | 1 | 60.12 |
| QWx1 | 1 | 35.16 |
| QWx1 | 2 | 60.57 |

The results show that it is possible to build up a cumulative dose and effect with multiple administrations over time, including at weekly or monthly intervals, to achieve increasing editing levels and % KD of TTR.

### Example 10. RNA Cargo: Varying mRNA and gRNA Ratios

This study evaluated in vivo efficacy in mice of different ratios of gRNA to mRNA. CleanCap™ capped Cas9 mRNAs with the ORF of SEQ ID NO: 4, HSD 5′ UTR, human albumin 3′ UTR, a Kozak sequence, and a poly-A tail were made by IVT synthesis as indicated in Example 1 with N1-methylpseudouridine triphosphate in place of uridine triphosphate.

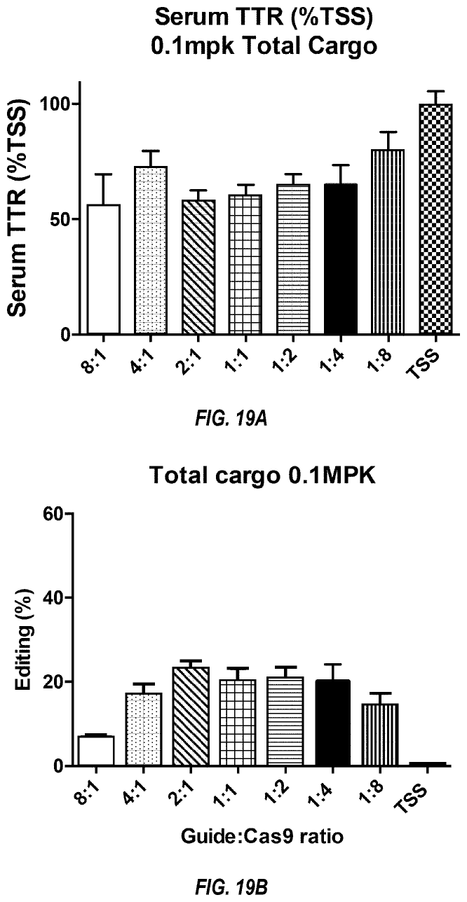

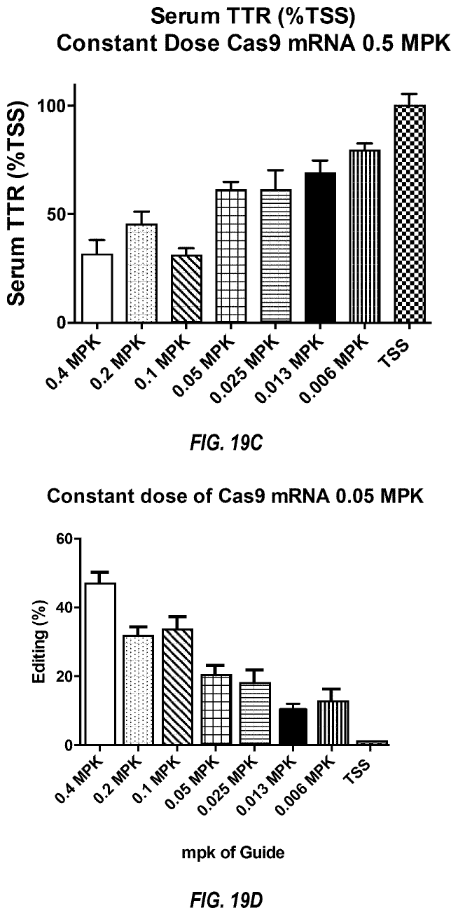

LNP formulations prepared from the mRNA described and G282 (SEQ ID NO: 124) as described in Example 1 with Lipid A, cholesterol, DSPC, and PEG2k-DMG in a 50:38:9:3 molar ratio and with an N:P ratio of 6. The gRNA:Cas9 mRNA weight ratios of the formulations were as shown in FIGS. 19A and 19B.

For in vivo characterization, the LNPs were administered to mice at 0.1 mg total RNA (mg guide RNA+mg mRNA) per kg (n=5 per group). At 7-9 days post-dose, animals were sacrificed, blood and the liver were collected, and serum TTR and liver editing were measured as described in Example 1. Serum TTR and liver editing results are shown in FIGS. 19A and 19B. Negative control mice were dosed with TSS vehicle.

In addition, the above LNPs were administered to mice at a constant mRNA dose of 0.05 mg mRNA per kg (n=5 per group), while varying the gRNA dose from 0.06 mg per kg to 0.4 mg per kg. At 7-9 days post-dose, animals were sacrificed, blood and the liver were collected, and serum TTR and liver editing were measured. Serum TTR and liver editing results are shown in FIG. 19C and FIG. 19D. Negative control mice were dosed with TSS vehicle.

### Example 11. Off-Target Analysis of TTR sgRNAs in Primary Human Hepatocytes

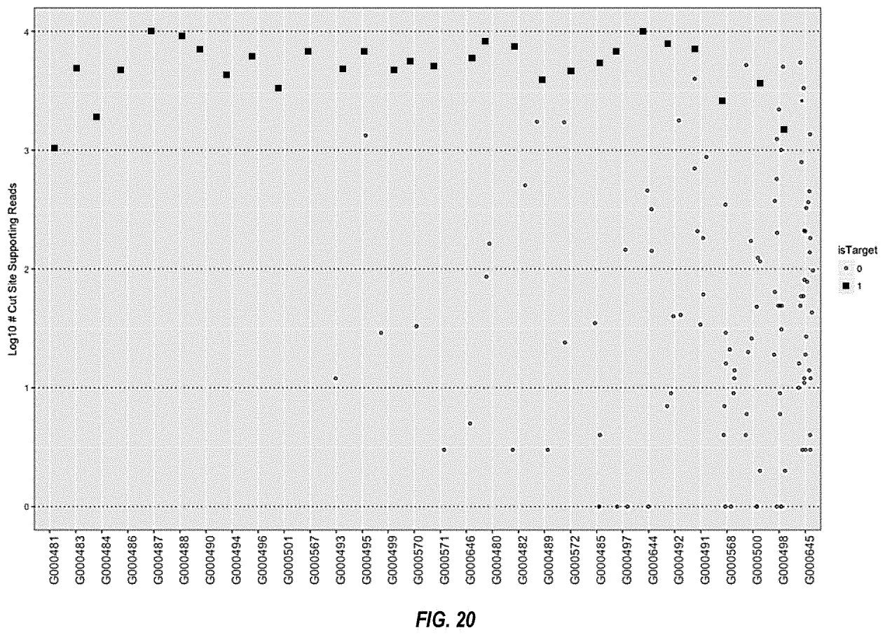

Off-target analysis of sgRNAs targeting TTR was performed in primary human hepatocytes (PHH) as described in Example 5, with the following modifications. PHH were plated at a density of 33,000 cells per well on collagen-coated 96-well plates as described in Example 1. Twenty-four hours post plating, cells were washed with media and transfected using Lipofectamine RNAiMAX (ThermoFisher, Cat. 13778150) as described in Example 1. Cells were transfected with a lipoplex containing 100 ng Cas9 mRNA, immediately followed by the addition of another lipoplex containing 25 nM of the sgRNA and 12.5 nM of the donor oligo (0.3 μL/well). Cells were lysed 48 hours post-transfection and gDNA was extracted and analyzed as further described in Example 5. The data is graphically represented in FIG. 20.

Table 32 shows the number of off-target integration sites detected in PHH, and compares to the number of sites that were detected in the HekCas9 cells used in Example 5. Fewer sites were detected in PHH for every guide tested as compared to the HekCas9 cell line, with no unique sites detected in PHH alone.

TABLE 32
Number of off-target integration sites detected for
TTR sgRNAs in PHH via an oligo insertion based assay

| | | # Sites in HekCas9 cells |
|---|---|---|
| GUIDE ID | # Sites in PHH | (Example 5) |
| G000480 | 2 | 11 |
| G000481 | 0 | 3 |
| G000482 | 2 | 13 |
| G000483 | 0 | 5 |
| G000484 | 0 | 7 |
| G000485 | 3 | 22 |
| G000486 | 0 | 12 |
| G000487 | 0 | 14 |
| G000488 | 0 | 0 |
| G000489 | 2 | 19 |
| G000490 | 0 | 12 |
| G000491 | 7 | 28 |
| G000492 | 5 | 97 |
| G000493 | 1 | 7 |
| G000494 | 0 | 4 |
| G000495 | 1 | 13 |
| G000496 | 0 | 1 |
| G000497 | 3 | 26 |
| G000498 | 19 | 82 |
| G000499 | 1 | 4 |
| G000500 | 12 | 46 |
| G000501 | 0 | 4 |
| G000567 | 0 | 9 |
| G000568 | 11 | 936 |
| G000570 | 1 | 19 |
| G000571 | 1 | 16 |
| G000572 | 2 | 15 |

Following the identification of potential off-target sites in PHH via the oligo insertion assay, certain potential sites were further evaluated by targeted amplicon sequencing, e.g., as described in Example 6. In addition to the potential off-target sites identified by the oligo insertion strategy, additional potential off-target sites identified by in silico prediction were included in the analysis.

To this end, PHH were treated with LNPs comprising 100 ng of Cas9 mRNA (SEQ ID NO:1) and the gRNA of interest at 14.68 nM (in a 1:1 ratio by weight), as described in Example 4. The LNPs were prepared using the cross-flow procedure described above and purified and concentrated using PD-10 columns and Amicon centrifugal filter units, respectively. The LNPs were formulated with an N:P ratio of 6.0 and contained Lipid A, Cholesterol, DSPC, and PEG2k-DMG in a 50:38:9:2 molar ratio, respectively. Following LNP treatment, isolated genomic DNA was analyzed by NGS (e.g., as described in Examples 1 and 6) to determine whether indels could be detected at the potential off-target site, which would be indicative of a Cas9-mediated cleavage event. Tables 33 and 34 show the potential off-target sites that were evaluated for the gRNAs G000480 and G000486, respectively.

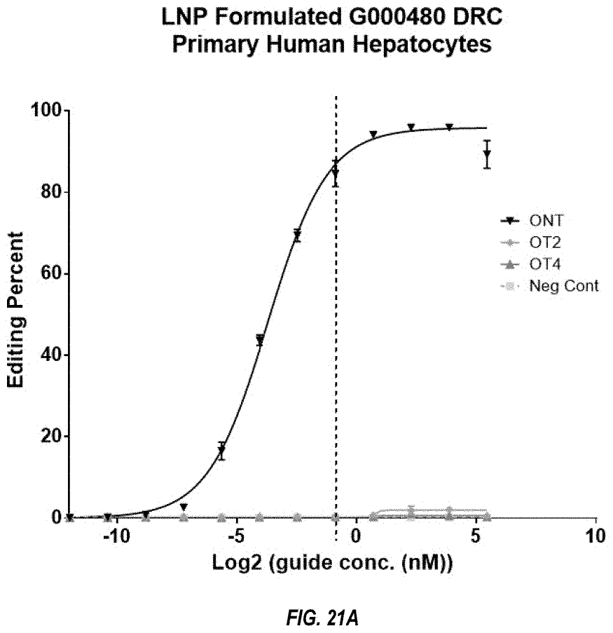

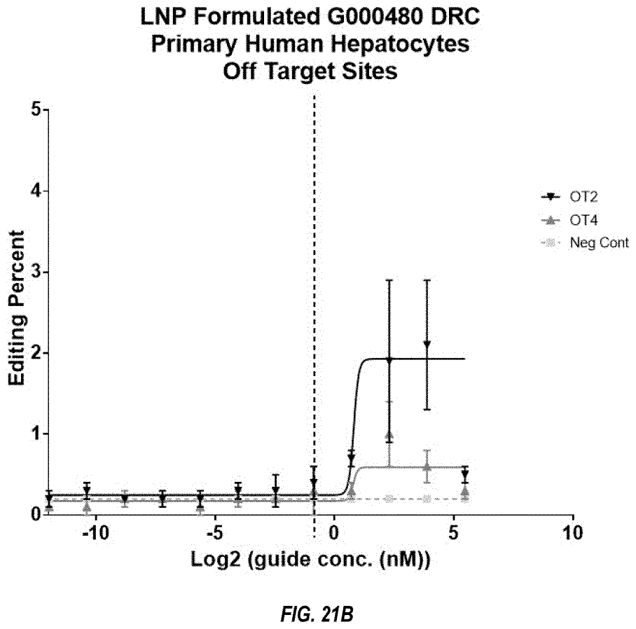

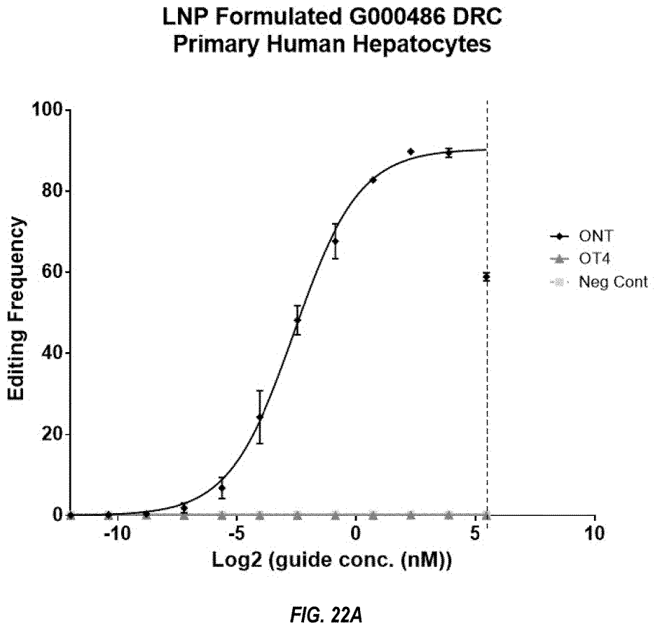

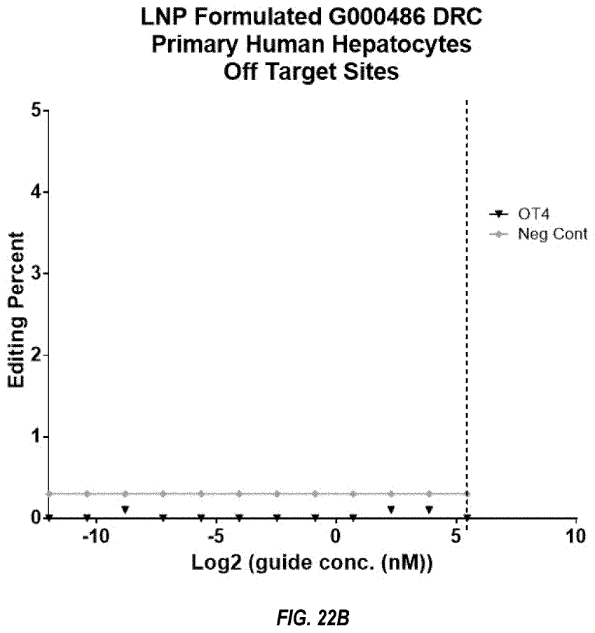

As shown in FIGS. 21A-B and 22A-B and Table 35 below, indels were detected at low levels for only two of the potential off-target sites identified by the oligo insertion assay for G000480, and only one for G000486. No indels were detected at any of the in silico predicted sites for either guide. Further, indels were only detected at these sites using a near-saturating dose of LNP, as the indel rates observed at the on-target sites for G000480 and G000486 were ˜97% and ˜91%, respectively (See Table 35). The genomic coordinates of these sites are also reported in Tables 33 and 34, and each correspond to sequences that do not code for any protein.

A dose response assay was then performed in order to determine the highest dose of LNP in which no off-targets were detected. PHH were treated with LNPs comprising either G000480 or G000486 as described in Example 4. The doses ranged across 11 points with respect to gRNA concentration (0.001 nM, 0.002 nM, 0.007 nM, 0.02 nM, 0.06 nM, 0.19 nM, 0.57 nM, 1.72 nM, 5.17 nM, 15.51 nM, and 46.55 nM). As represented by the dashed vertical line in FIGS. 21A-B and 22A-B, the highest concentrations (with respect to the concentration of gRNA) at which the potential off-target sites were no longer detected for G000480 and G000486 were 0.57 nM and 15.51 nM, respectively, which resulted in on-target indel rates of 84.60% and 89.50%, respectively.

TABLE 33
Identified potential off target sites via insertion detection and in silico
prediction for G000480 evaluated via targeted amplicon sequencing

| | Off-target | | Chromosomal Coordinates | |
|---|---|---|---|---|
| GUIDE ID | (OT) Site ID | Assay Used | (hg38) | Strand |
| G000480 | INS-OT.1 | Insertion Detection | chr7: 94767406-94767426 | + |
| G000480 | INS-OT.2 | Insertion Detection | chr2: 192658562-192658582 | + |
| G000480 | INS-OT.3 | Insertion Detection | chr7: 4834390-4834410 | + |
| G000480 | INS-OT.4 | Insertion Detection | chr20: 9216118-9216138 | − |
| G000480 | INS-OT.5 | Insertion Detection | chr10: 12547071-12547091 | + |
| G000480 | INS-OT.6 | Insertion Detection | chr6: 168377978-168377998 | − |
| G000480 | INS-OT.7 | Insertion Detection | chr12: 114144669-114144689 | − |
| G000480 | INS-OT.8 | Insertion Detection | chr10: 7376755-7376775 | + |
| G000480 | INS-OT.9 | Insertion Detection | chr2: 52950299-52950319 | + |
| G000480 | INS-OT.10 | Insertion Detection | chr8: 56579165-56579185 | − |
| G000480 | INS-OT.11 | Insertion Detection | chr1: 189992255-189992275 | + |
| G000480 | PRED-OT.1 | in silico prediction | chr10: 12547071-12547091 | + |
| G000480 | PRE-DOT.2 | in silico prediction | chrX: 119702782-119702802 | + |
| G000480 | PRED-OT.3 | in silico prediction | chr1: 116544586-116544606 | + |
| G000480 | PRED-OT.4 | in silico prediction | chr6: 88282884-88282904 | + |
| G000480 | PRED-OT.6 | in silico prediction | chr5: 121891868-121891888 | + |
| G000480 | PRED-OT.7 | in silico prediction | chr3: 52544945-52544965 | + |
| G000480 | PRED-OT.8 | in silico prediction | chr15: 36949639-36949659 | + |
| G000480 | PRED-OT.9 | in silico prediction | chr5: 33866486-33866506 | + |
| G000480 | PRED-OT.10 | in silico prediction | chr5: 159755754-159755774 | + |
| G000480 | PRED-OT.11 | in silico prediction | chr5: 31349859-31349879 | + |
| G000480 | PRED-OT.12 | in silico prediction | chr11: 79485652-79485672 | + |
| G000480 | PRED-OT.13 | in silico prediction | chr15: 29448864-29448884 | + |
| G000480 | PRED-OT.14 | in silico prediction | chr5: 171153565-171153585 | + |
| G000480 | PRED-OT.15 | in silico prediction | chr9: 84855273-84855293 | + |
| G000480 | PRED-OT.16 | in silico prediction | chr6: 159953060-159953080 | + |
| G000480 | PRED-OT.17 | in silico prediction | chr16: 51849024-51849044 | + |
| G000480 | PRED-OT.18 | in silico prediction | chr3: 24108809-24108829 | + |
| G000480 | PRED-OT.19 | in silico prediction | chr18: 41118310-41118330 | + |
| G000480 | PRED-OT.20 | in silico prediction | chr10: 108975241-108975261 | + |
| G000480 | PREDO-T.21 | in silico prediction | chr1: 44683633-44683653 | + |
| G000480 | PRED-OT.22 | in silico prediction | chr2: 196214849-196214869 | + |
| G000480 | PRED-OT.23 | in silico prediction | chr9: 117353544-117353564 | + |
| G000480 | PRED-OT.24 | in silico prediction | chr1: 55583322-55583342 | + |
| G000480 | PRED-OT.25 | in silico prediction | chr12: 28246827-28246847 | + |
| G000480 | PRED-OT.26 | in silico prediction | chr4: 54545361-54545381 | + |
| G000480 | PRED-OT.27 | in silico prediction | chr13: 22364836-22364856 | + |
| G000480 | PRED-OT.28 | in silico prediction | chr13: 80816049-80816069 | + |
| G000480 | PRED-OT.29 | in silico prediction | chr7: 39078622-39078642 | + |
| G000480 | PRED-OT.30 | in silico prediction | chr2: 59944386-59944406 | + |

“INS-OT.N” refers to an off-target site ID detected by oligo insertion, where N is an integer specified above; “PRED-OT.N refers to an off-target site ID predicted via in silico methods, where N is an integer specified above.

TABLE 34
Identified potential off target sites via insertion detection and in silico
prediction for G000486 evaluated via targeted amplicon sequencing

| | Off-target | | Chromosomal Coordinates | |
|---|---|---|---|---|
| GUIDE ID | (OT) Site ID | Assay Used | (hg38) | Strand |
| G000486 | INS-OT.1 | Insertion Detection | chr14: 77332157-77332177 | + |
| G000486 | INS-OT.2 | Insertion Detection | chr14: 54672059-54672079 | − |
| G000486 | INS-OT.3 | Insertion Detection | chr4: 108513169-108513189 | − |
| G000486 | INS-OT.4 | Insertion Detection | chr5: 91397023-91397043 | − |
| G000486 | INS-OT.5 | Insertion Detection | chr9: 116626135-116626155 | − |
| G000486 | INS-OT.6 | Insertion Detection | chr6: 73201226-73201246 | + |
| G000486 | INS-OT.7 | Insertion Detection | chr16: 89368352-89368372 | − |
| G000486 | INS-OT.8 | Insertion Detection | chr7: 56308371-56308391 | − |
| G000486 | INS-OT.9 | Insertion Detection | chr21: 43605667-43605687 | + |
| G000486 | INS-OT.10 | Insertion Detection | chr5: 26758030-26758050 | + |
| G000486 | INS-OT.11 | Insertion Detection | chr17: 30656428-30656448 | + |
| G000486 | INS-OT.12 | Insertion Detection | chr8: 130486452-A130486472 | + |
| G000486 | PRED-OT.1 | in silico prediction | chr11: 44707064-44707084 | + |
| G000486 | PRED-OT.2 | in silico prediction | chr5: 50775396-50775416 | + |
| G000486 | PRED-OT.3 | in silico prediction | chr4: 141623949-141623969 | + |
| G000486 | PRED-OT.4 | in silico prediction | chr1: 223481186-223481206 | + |
| G000486 | PRED-OT.5 | in silico prediction | chr6: 39951487-39951507 | + |
| G000486 | PRED-OT.6 | in silico prediction | chrY: 5456047-5456067 | + |
| G000486 | PRED-OT.8 | in silico prediction | chr6: 129868719-129868739 | + |
| G000486 | PRED-OT.9 | in silico prediction | chrX: 80450312-80450332 | + |
| G000486 | PRED-OT.10 | in silico prediction | chr7: 27256771-27256791 | + |
| G000486 | PRED-OT.11 | in silico prediction | chr3: 181416528-181416548 | + |
| G000486 | PRED-OT12 | in silico prediction | chr7: 146425020-146425040 | + |
| G000486 | PRED-OT.13 | in silico prediction | chr3: 16980977-16980997 | + |
| G000486 | PRED-OT.14 | in silico prediction | chr7: 118161002-118161022 | + |
| G000486 | PRED-OT.15 | in silico prediction | chr6: 102220539-102220559 | + |
| G000486 | PRED-OT.16 | in silico prediction | chr12: 127278991-127279011 | + |
| G000486 | PRED-OT.17 | in silico prediction | chr2: 67686631-67686651 | + |
| G000486 | PRED-OT.18 | in silico prediction | chr1: 114467665-114467685 | + |
| G000486 | PRED-OT.19 | in silico prediction | chr3: 194514436-194514456 | + |
| G000486 | PRED-OT.20 | in silico prediction | chr14: 31767581-31767601 | + |
| G000486 | PRED-OT.21 | in silico prediction | chr16: 28706209-28706229 | + |
| G000486 | PRED-OT.22 | in silico prediction | chr8: 110526279-110526299 | + |
| G000486 | PRED-OT.23 | in silico prediction | chr19: 2899814-2899834 | + |
| G000486 | PRED-OT.25 | in silico prediction | chr3: 130760261-A130760281 | + |
| G000486 | PRED-OT.26 | in silico prediction | chr11: 2506046-2506066 | + |
| G000486 | PRED-OT.27 | in silico prediction | chr2: 153918318-153918338 | + |
| G000486 | PRED-OT.28 | in silico prediction | chr14: 40590226-40590246 | + |
| G000486 | PRED-OT.29 | in silico prediction | chr18: 806650-806670 | + |
| G000486 | PRED-OT.30 | in silico prediction | chr2: 117707480-117707500 | + |

“INS-OT.N” refers to an off-target site ID detected by oligo insertion, where N is an integer specified above; “PRED-OT.N” refers to an off-target site ID predicted via in silico methods, where N is an integer specified.

TABLE 35
Detected Off Target sites in PHH treated with
LNP containing 100 ng mRNA and 31.03 nM gRNA

| | | | Indel Frequency | |
|---|---|---|---|---|
| | Off-target | | (using LNP with | Indel |
| | (OT) Site | Site | 100 ng Cas9 mRNA | Frequency |
| GUIDE ID | ID | Type | and 14.68 nM gRNA) | std. dev. |
| G000480 | n/a | On-Target | 97.33% | 1.10% |
| G000480 | INS-OT.2 | Off-Target | 1.43% | 0.40% |
| G000480 | INS-OT.4 | Off-Target | 0.97% | 0.25% |
| G000486 | n/a | On-Target | 91.33% | 1.97% |
| G000486 | INS-OT.4 | Off-Target | 0.47% | 0.06% |

### Example 12. LNP Delivery to Humanized Mouse Model of ATTR

A well-established humanized transgenic mouse model of hereditary ATTR amyloidosis that expresses the V30M pathogenic mutant form of human TTR protein was used in this Example. This mouse model recapitulates the TTR deposition phenotype in tissues observed in ATTR patients, including within the peripheral nervous system and gastrointestinal (GI) tract (See Santos et al., Neurobiol Aging. 2010 February; 31(2):280-9).

Mice (aged approximately 4-5 months) were dosed with LNP formulations prepared using the cross-flow and TFF procedures as described in Example 1. The LNPs were formulated with an N:P ratio of 6.0 and contained Lipid A, Cholesterol, DSPC, and PEG2k-DMG in a 50:38:9:2 molar ratio, respectively. The LNPs contained Cas9 mRNA (SEQ ID NO: 1) and either G000481 (“G481”) or a non-targeting control guide G000395 (“G395”; SEQ ID NO: 273), in a 1:1 ratio of gRNA:mRNA by weight.

Mice were injected via the lateral tail vein as described in Example 1 with a single 1 mg/kg (of total RNA content) dose of LNP with an n=10/group. At 8 weeks post treatment, the mice were euthanized for sample collection. Human TTR protein levels were measured in serum and cerebrospinal fluid (CSF) by ELISA as previously described by Butler et al., Amyloid. 2016 June; 23(2):109-18. Liver tissue was assayed for editing levels as described in Example 1. Other tissues (stomach, colon, sciatic nerve, dorsal root ganglion (DRG)) were collected and processed for semi-quantitative immunohistochemistry as previously described by Gonçalves et al., Amyloid. 2014 September; 21(3): 175-184. Statistical analysis for the immunohistochemistry data was performed using Mann Whitney test with a p-value<0.0001.

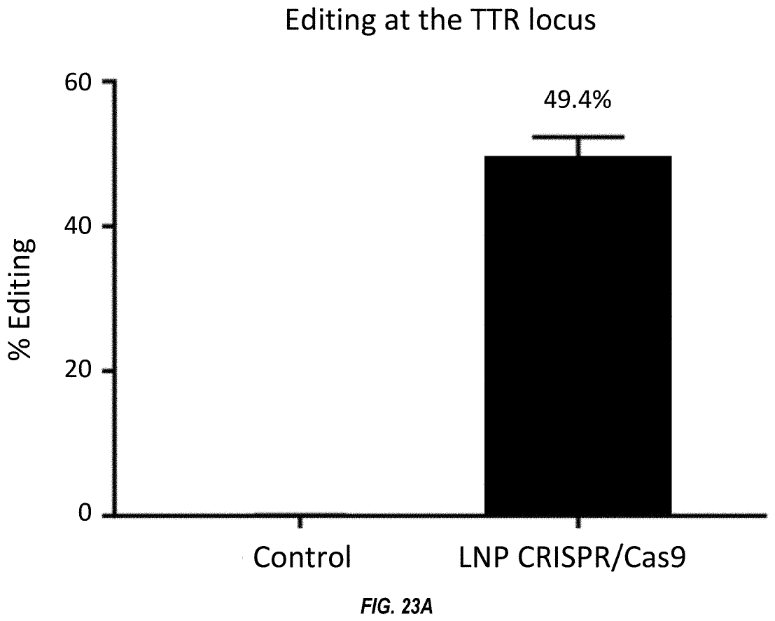

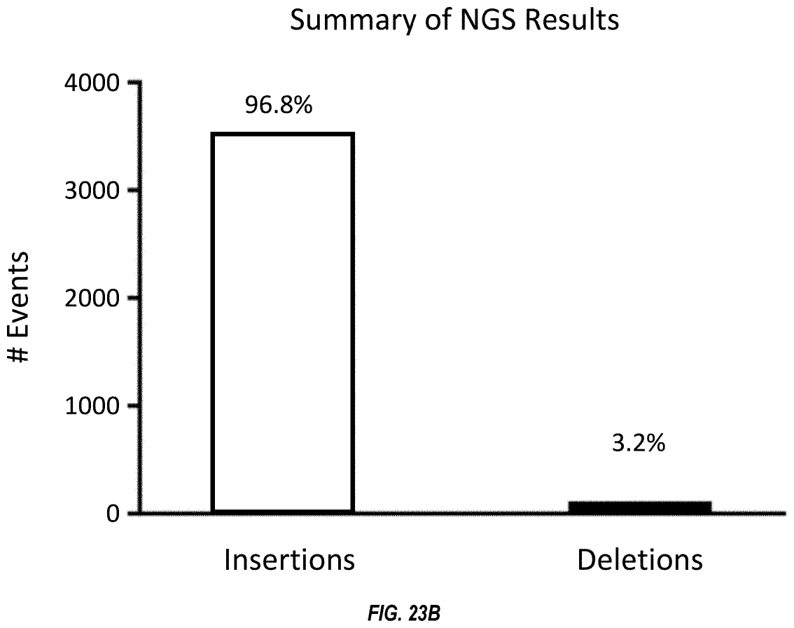

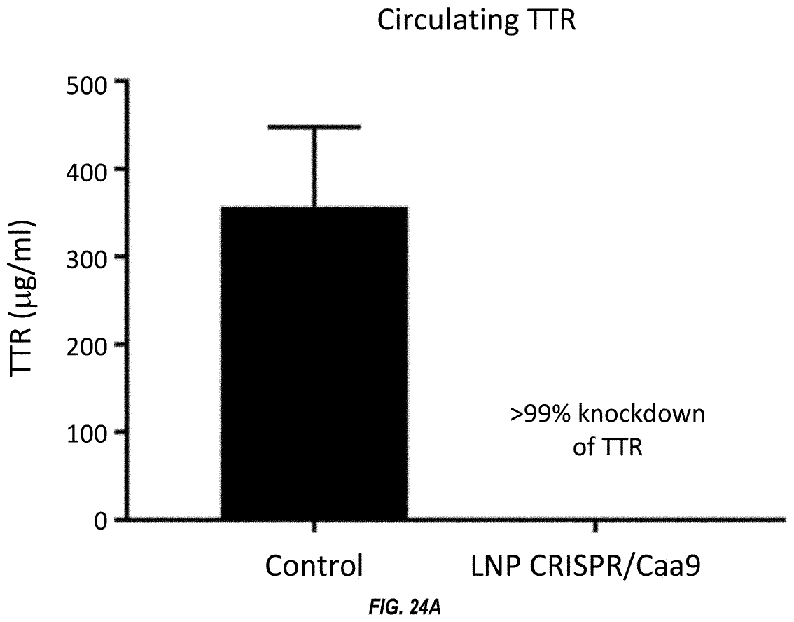

As shown in FIG. 23A-B, robust editing (49.4%) of TTR was observed in livers of the humanized mice following the single dose of LNP comprising G481, with no editing detected in the control group. Analysis of the editing events demonstrated that 96.8% of the events were insertions, with the remainder deletions.

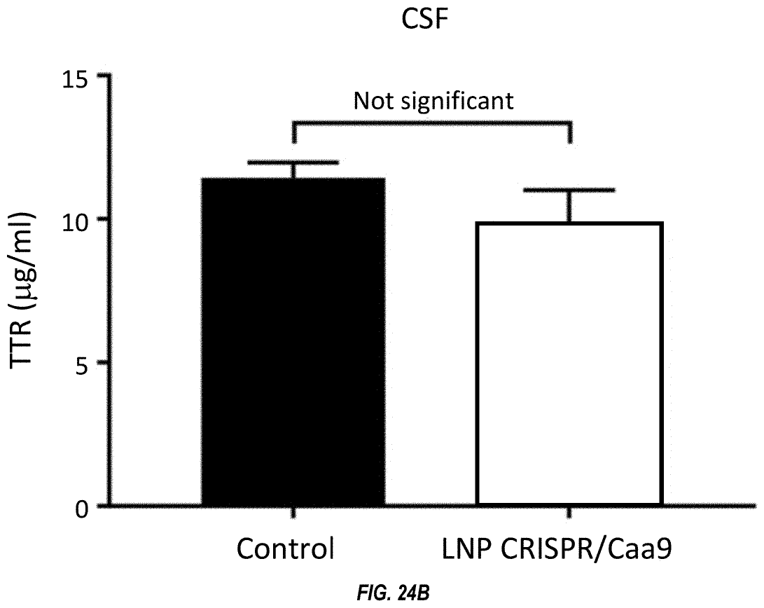

As shown in FIG. 24A-B, TTR protein levels were decreased in plasma but not in CSF from the treated mice, with greater than 99% knockdown of TTR plasma levels observed (p<0.001).

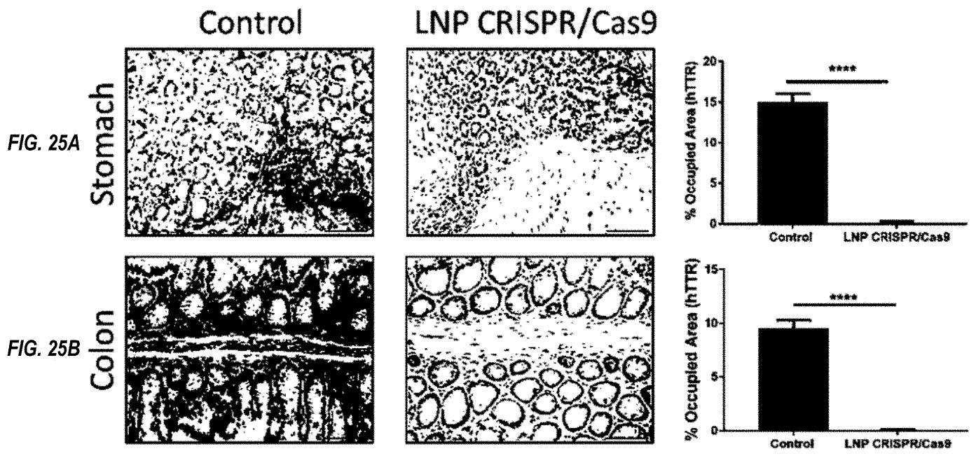

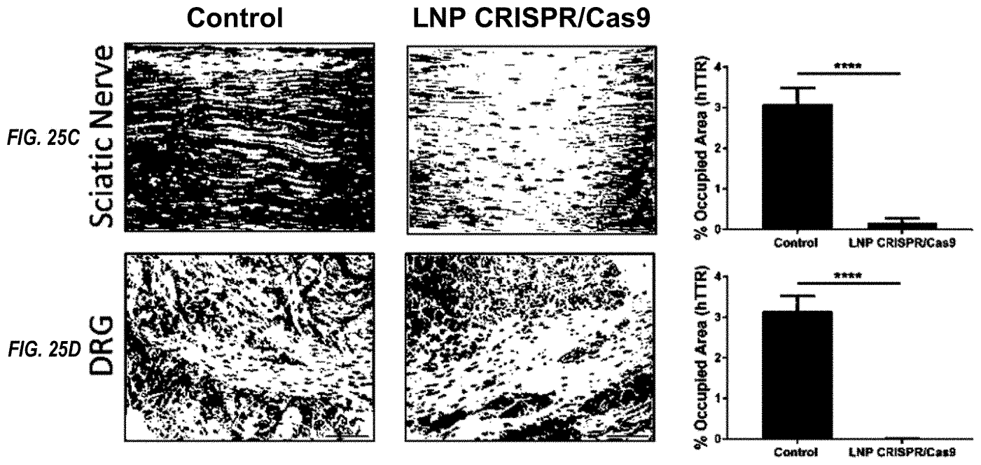

The near complete knockdown of TTR observed in the plasma of treated animals correlated with the clearance of TTR protein amyloid deposition in the assayed tissues. As shown in FIG. 25, control mice exhibited amyloid staining in tissues which resembles the pathophysiology observed in human subjects with ATTR. Decreasing circulating TTR by editing the HuTTR V30M locus resulted in a dramatic decrease of amyloid deposition in tissues. Approximately 85% or better reduction in TTR staining was observed across the treated tissues 8 weeks post-treatment (FIG. 25).

### Example 13. TTR mRNA Knockdown in Primary Human Hepatocytes (PHH)

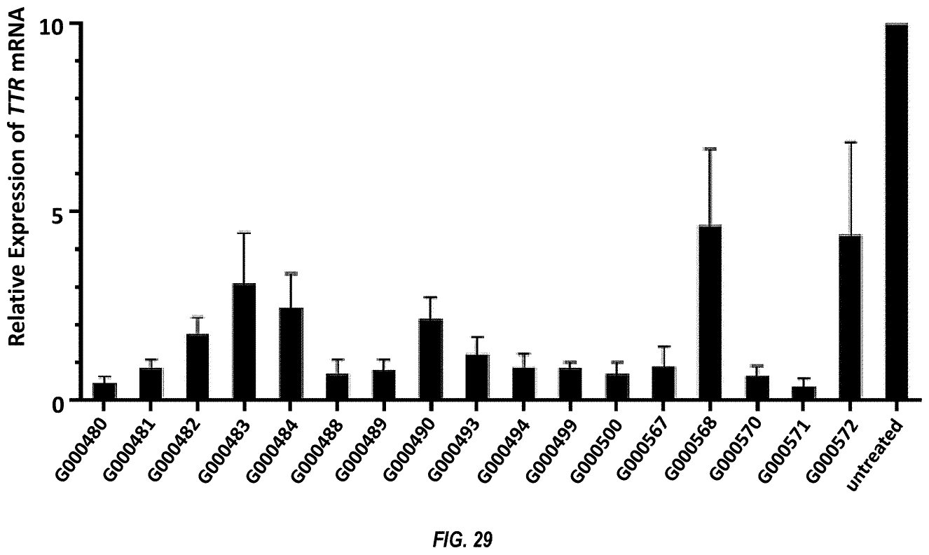

In one experiment, PHH were cultured and treated with LNPs comprising Cas9 mRNA (SEQ ID NO:1) and a gRNA of interest (See FIG. 29, Table 36), as described in Example 4. The LNPs were prepared using the cross-flow procedure described above and purified and concentrated using PD-10 columns and Amicon centrifugal filter units, respectively. The LNPs were formulated with an N:P ratio of 6.0 and contained Lipid A, Cholesterol, DSPC, and PEG2k-DMG in a 50:38:9:2 molar ratio, respectively. The LNPs comprised a gRNA:mRNA ratio of 1:2, and the cells were treated at a dose of 300 ng (with respect to the amount of mRNA cargo delivered).

Ninety-six (96) hours following LNP treatment (with biological triplicates for each condition), mRNA was purified from PHH cells using the Dynabeads mRNA DIRECT Kit (ThermoFisher Scientific) according to the manufacturer's protocol. Reverse Transcription (RT) was performed with Maxima reverse transcriptase (ThermoFisher Scientific) and a poly-dT primer. The resulting cDNA was purified with Ampure XP Beads (Agencourt). For Quantitative PCR, 2% of the purified cDNA was amplified with Taqman Fast Advanced Mastermix and 3 Taqman probe sets, TTR (Assay ID: Hs00174914_m1), GAPDH (Assay ID: Hs02786624_g1), and PPIB (Assay ID: Hs00168719_m1). The assays were run on the QuantStudio 7 Flex Real Time PCR System according to the manufacturer's instructions (Life Technologies). Relative expression of TTR mRNA was calculated by normalizing to the endogenous controls (GAPDH and PPIB) individually, and then averaged.

As shown in FIG. 29 and reproduced numerically in Table 36 below, each of the LNP formulations tested resulted in knockdown of TTR mRNA, as compared to the negative (untreated) control. The groups in FIG. 29 and Table 36 are identified by the gRNA ID used in each LNP preparation. Relative expression of TTR mRNA is plotted in FIG. 29, whereas the percent knockdown of TTR mRNA is provided in Table 36.

TABLE 36

| GUIDE ID | Avg % Knockdown | Std Dev |
|---|---|---|
| G000480 | 95.19 | 1.68 |
| G000481 | 91.39 | 2.39 |
| G000482 | 82.31 | 4.51 |
| G000483 | 68.78 | 13.45 |
| G000484 | 75.22 | 9.05 |
| G000488 | 92.77 | 3.76 |
| G000489 | 91.85 | 2.77 |
| G000490 | 78.34 | 5.76 |
| G000493 | 87.53 | 4.54 |
| G000494 | 91.15 | 3.63 |
| G000499 | 91.38 | 1.71 |
| G000500 | 92.90 | 3.15 |
| G000567 | 90.89 | 5.39 |
| G000568 | 53.44 | 20.20 |
| G000570 | 93.38 | 2.66 |
| G000571 | 96.17 | 2.07 |
| G000572 | 55.92 | 24.53 |

In a separate experiment, TTR mRNA knockdown was evaluated following treatment with LNPs comprising G000480, G000486, and G000502. The LNPs were formulated and PHH were cultured and treated with the LNPs, each as described in the experiment above in this Example with the exception that the cells were treated at a dose of 100 ng (with respect to the amount of mRNA cargo delivered).

Ninety-six (96) hours following LNP treatment (single treatment for each condition), mRNA was purified from PHH cells using the Dynabeads mRNA DIRECT Kit (ThermoFisher Scientific) according to the manufacturer's protocol. Reverse Transcription (RT) was performed with the High Capacity cDNA Reverse Transcription Kit (ThermoFisher Scientific) according to the manufacturer's instructions. For Quantitative PCR, 2% of the cDNA was amplified with Taqman Fast Advanced Mastermix and 3 Taqman probe sets, TTR (Assay ID: Hs00174914_m1), GAPDH (Assay ID: Hs02786624_g1), and PPIB (Assay ID: Hs00168719_m1). The assays were run on the QuantStudio 7 Flex Real Time PCR System according to the manufacturer's instructions (Life Technologies). Relative expression of TTR mRNA was calculated by normalizing to the endogenous controls (GAPDH and PPIB) individually, and then averaged.

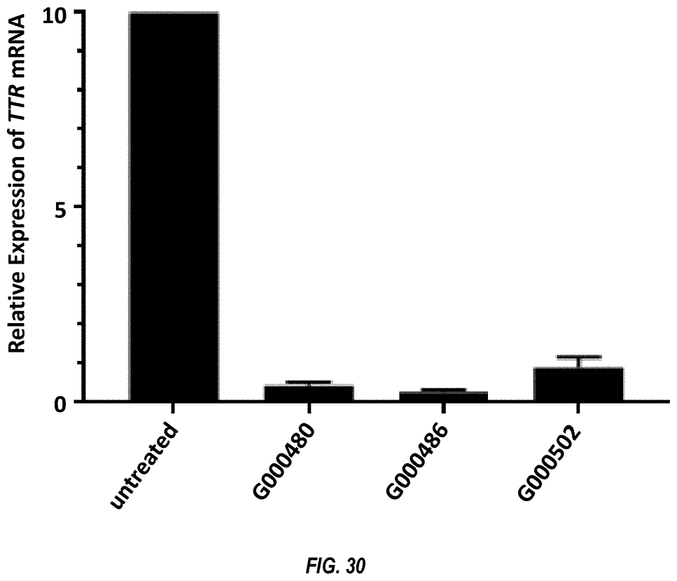

As shown in FIG. 30 and reproduced numerically in Table 37 below, each of the LNP formulations tested resulted in knockdown of TTR mRNA, as compared to the negative (untreated) control. The groups in FIG. 30 and Table 37 are identified by the gRNA ID used in each LNP preparation. Relative expression of TTR mRNA is plotted in FIG. 30, whereas the percent knockdown of TTR mRNA is provided in Table 37.

TABLE 37

| GUIDE ID | Avg % Knockdown | Std Dev |
|---|---|---|
| G000480 | 95.61 | 0.92 |
| G000486 | 97.36 | 0.63 |
| G000502 | 90.94 | 2.63 |

### Example 14. Corticosteroid Pre-Treatment and LNP Delivery to Non-Human Primates

Male cynomologus monkeys in cohorts of n=3 were treated with dexamethasone and varying doses of LNP to provide 1 mg/kg, 3 mg/kg, or 6 mg/kg (RNA) per NHP. Each formulation contained Cas9 mRNA000042 (SEQ ID No. 377) and guide RNA (gRNA) G000502 (SEQ ID No. 114) in a gRNA:mRNA ratio of 1:2 by weight. Except for animals treated with vehicle control, all animals received dexamethasone (Dex) pre-treatment at 2 mg/kg by IV bolus injection 1-2 hours prior to LNP administration. Doses of LNP (in mg/kg, total RNA content), were administered by 30 minute IV infusion.

At day 15 post-dose, liver specimens were collected through single ultrasound-guided percutaneous biopsy targeting the right lobe/side of the liver, using a 16-gauge SuperCore biopsy needle. A minimum of 1.5 cm³ of total liver biopsy were collected per animal. Each biopsy specimen was flash frozen in liquid nitrogen and stored at −86 to −60° C. Editing analysis of the liver specimens was performed through NGS sequencing as previously described. Results for the liver editing demonstrated up to about 70% editing with all doses well tolerated. Corticosteroid pre-treatment with the described LNP treatment was well tolerated.

Materials and Methods for Example 14. mRNA was synthesized by in vitro transcription (IVT) using a linearized plasmid DNA template and T7 RNA polymerase. Transcription was generally performed from constructs comprising a T7 Promoter (SEQ ID NO: 231), a transcript sequence disclosed herein such as SEQ ID NO: 377 (which encodes the RNA ORF of SEQ ID NO: 311), and a poly-A tail (SEQ ID NO: 263) encoded in the plasmid.

For all methods, the transcript concentration was determined by measuring the light absorbance at 260 nm (Nanodrop), and the transcript was analyzed by capillary electrophoresis by Bioanalyzer (Agilent).

LNP Formulation

The lipid components were dissolved in 100% ethanol with the lipid component molar ratios described below. The chemically modified sgRNA and Cas9 mRNA were combined and dissolved in 25 mM citrate, 100 mM NaCl, pH 5.0, resulting in a concentration of total RNA cargo of approximately 1.5 mg/mL. The LNPs were formulated with an N/P ratio of about 6, with the ratio of chemically modified sgRNA:Cas9 mRNA at a 1:2 w/w ratio as described below. LNPs were formulated with 50% Lipid A, 9% DSPC, 38% cholesterol, and 3% PEG2k-DMG, and LNPs were formed by cross-flow technique as described in Example 1. During mixing, a 2:1 ratio of aqueous to organic solvent was maintained using differential flow rates. Diluted LNPs were concentrated using tangential flow filtration and then buffer exchanged by diafiltration prior to filtering and storage.

Cas9 mRNA and gRNA Cargos

Capped and polyadenylated Cas9 mRNA was generated by in vitro transcription using a linearized plasmid DNA template and T7 RNA polymerase using the method described in Example 1.

### Genomic DNA Isolation

Genomic DNA was extracted from liver samples using 50 μL/well BuccalAmp DNA Extraction solution (Epicentre, Cat. QE09050) according to manufacturer's protocol. All DNA samples were subjected to PCR and subsequent NGS analysis, as described herein.

### NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., TTR), and the genomic area of interest was amplified. Primer sequences are provided below. Additional PCR was performed according to the manufacturer's protocols (Illumina) to add the necessary chemistry for sequencing. The amplicons were sequenced on an Illumina MiSeq instrument. The reads were aligned to a cyno reference genome (e.g., macFas5) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

### Example 15: Multiple Dose LNP Study Administered Via 30 Minute and 2 Hour IV Infusion in Cynomolgus Monkeys

Male cynomolgus monkeys in cohorts of n=3 were administered dexamethasone (Dex) via IV bolus injection at 2 mg/kg a minimum of 1 hour prior to LNP or vehicle control administration. Each cohort received varying doses of LNP to provide 3 mg/kg, or 6 mg/kg (RNA) per NHP. Dosing groups are shown in Table 38. Two cohorts received an LNP dose of 3 mg/kg in order to compare infusion time. Formulations contained Cas9 mRNA and guide RNA were prepared as described below and in Example 14. The LNP formulations were prepared as described below and in Example 14. The cohorts receiving an LNP dose of 3 mg/kg (total RNA content), were administered by 30-minute or 120-minute IV infusion. All other cohorts with various doses of LNP (in mg/kg, total RNA content), were administered by 120-minute IV infusion.

TABLE 38
Infusion Study Dosing Groups

| | | Dose | | |
|---|---|---|---|---|
| Group | | Level | Infusion | |
| Number | Test Material | (mg/kg) | Time (min) | # of Animals |
| 1 | TSS Control | 0 | 120 | 3 |
| 2 | LNP | 3.0 | 120 | 3 |
| 3 | LNP | 3.0 | 30 | 3 |
| 4 | LNP | 6.0 | 120 | 3 |

TABLE 39
% Editing and Serum TTR

| | | |
|---|---|---|
| Group | | |
| Number | Liver Editing (%) | TTR % Reduction |

| | | | | |
|---|---|---|---|---|
| 1 | 0.0 | (0.0, 0.0, 0.0) | −28 | (−34, −23, −27) |
| 2 | 63.3 | (50.8, 69.0, 69.9) | 85 | (66, 95, 94) |
| 3 | 63.3 | (65.0, 66.0, 58.8) | 88 | (90, 89, 86) |
| 4 | 74.5 | (75.3, 74.6, 73.6) | 96 | (97, 96, 95) |

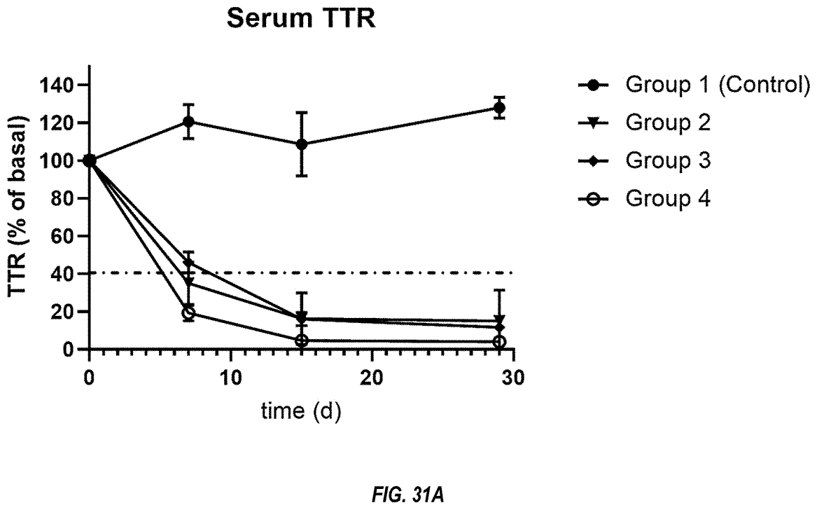

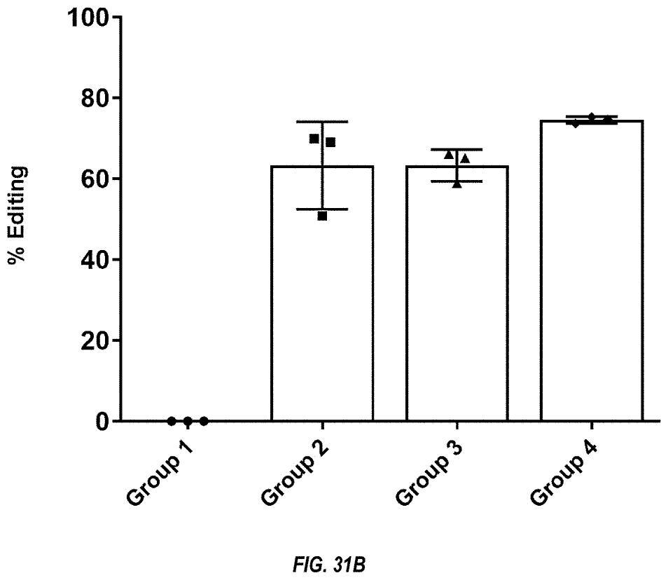

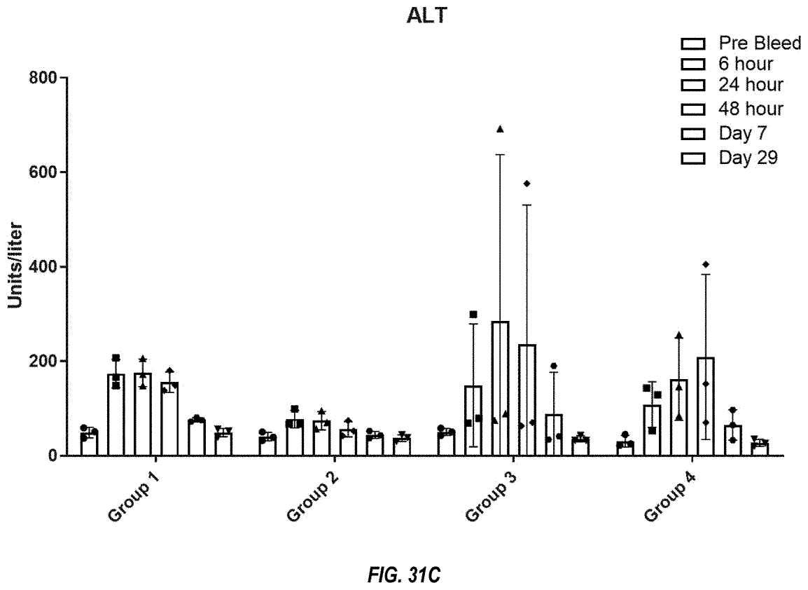

At day 29 post-dose, liver specimens were collected through single ultrasound-guided percutaneous biopsy targeting the right lobe/side of the liver, using a 16-gauge SuperCore biopsy needle under an intramuscular injection of ketamine/xylazine. A sample between 1.0 cm³ and 1.5 cm³ of total liver biopsy were collected per animal. Each biopsy specimen was flash frozen in liquid nitrogen and stored at −80° C. Editing analysis of the liver specimens was performed through NGS sequencing as previously described and is shown in FIG. 31B. Results for the liver editing demonstrated up to about 70% editing. Serum TTR levels are depicted in FIG. 31A. Corticosteroid pre-treatment with the described LNP treatment was well tolerated.

TABLE 40
Alanine Transaminase (ALT) Levels

| | Pre- Bleed | | 6 Hour | | 24 Hour | | 48 Hour | | Day 7 | | Day 29 | |
|---|---|---|---|---|---|---|---|---|---|---|---|---|
| Group | Avg | SD | Avg | SD | Avg | SD | Avg | SD | Avg | SD | Avg | SD |
| Group 1: TSS | 49.0 | 11.1 | 173.6 | 30.2 | 175.3 | 29.1 | 155.6 | 21.7 | 76.0 | 4.5 | 49.0 | 8.8 |
| Group 2: 3 mpk, | 40.3 | 9.0 | 77.6 | 18.4 | 74.0 | 19.3 | 56.0 | 16.3 | 44.0 | 7.5 | 37.3 | 7.0 |
| 2 hr infusion | | | | | | | | | | | | |
| Group 3: 3 mpk, | 50.3 | 7.5 | 149.0 | 130.0 | 285.3 | 352.2 | 236.3 | 294.1 | 88.3 | 88.1 | 35.6 | 6.3 |
| 30 min infusion | | | | | | | | | | | | |
| Group 4: 6 mpk, | 30.6 | 12.5 | 108.3 | 48.4 | 162.0 | 87.1 | 209.0 | 174.6 | 65.0 | 32.0 | 27.0 | 7.5 |
| 2 hr infusion | | | | | | | | | | | | |

Samples were analyzed for percent editing data, serum TTR data, and alanine transaminase (ALT) levels as shown in Table 39 and FIGS. 31A-B, and Table 40 and FIG. 31C, respectively. Results for the liver editing and serum TTR data demonstrate that there is no significant difference in potency between the 3 mg/kg dose with a 30 minute infusion time and a 3 mg/kg dose with a 120 minute infusion time. The greater than 30′ infusion time administrations, however, demonstrate lower levels of ALT, a liver injury biomarker. ALT levels were observed to be higher in the 3 mg/kg dose with a 30 minute infusion time which indicated potential liver stress.

Materials and Methods for Example 4. mRNA was synthesized by in vitro transcription (IVT) using a linearized plasmid DNA template and T7 RNA polymerase. Transcription was generally performed from constructs comprising a T7 Promoter (SEQ ID NO: 231), a transcript sequence disclosed herein such as SEQ ID NO: 377 (which encodes the RNA ORF of SEQ ID NO: 311), and a poly-A tail (SEQ ID NO: 263) encoded in the plasmid.

For all methods, the transcript concentration was determined by measuring the light absorbance at 260 nm (Nanodrop), and the transcript was analyzed by capillary electrophoresis by Bioanalyzer (Agilent).

LNP Formulation

The lipid components were dissolved in 100% ethanol with the lipid component molar ratios described below. The chemically modified sgRNA and Cas9 mRNA were combined and dissolved in 25 mM citrate, 100 mM NaCl, pH 5.0, resulting in a concentration of total RNA cargo of approximately 1.5 mg/mL. The LNPs were formulated with an N/P ratio of about 6, with the ratio of chemically modified sgRNA:Cas9 mRNA at a 1:2 w/w ratio as described below. LNPs were formulated with 50% Lipid A, 9% DSPC, 38% cholesterol, and 3% PEG2k-DMG, and LNPs were formed by cross-flow technique as described in Example 1. During mixing, a 2:1 ratio of aqueous to organic solvent was maintained using differential flow rates. Diluted LNPs were concentrated using tangential flow filtration and then buffer exchanged by diafiltration prior to filtering and storage.

Cas9 mRNA and gRNA Cargos

Capped and polyadenylated Cas9 mRNA was generated by in vitro transcription using a linearized plasmid DNA template and T7 RNA polymerase using the method described in Example 1.

### Genomic DNA Isolation

Genomic DNA was extracted from liver samples using 50 μL/well BuccalAmp DNA Extraction solution (Epicentre, Cat. QE09050) according to manufacturer's protocol. All DNA samples were subjected to PCR and subsequent NGS analysis, as described herein.

### NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., TTR), and the genomic area of interest was amplified. Primer sequences are provided below. Additional PCR was performed according to the manufacturer's protocols (Illumina) to add the necessary chemistry for sequencing. The amplicons were sequenced on an Illumina MiSeq instrument. The reads were aligned to a cyno reference genome (e.g., macFas5) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

### Example 16: Additional Numbered Embodiments

The following additional embodiments are provided.

Embodiment A1 is a composition comprising:

(i) a nucleic acid comprising an open reading frame encoding an RNA-guided DNA binding agent, wherein:
a. the open reading frame comprises a sequence with at least 93% identity to SEQ ID NO: 311; and/or
b. the open reading frame has at least 93% identity to SEQ ID NO: 311 over at least its first 50, 200, 250, or 300 nucleotides, or at least 95% identity to SEQ ID NO: 311 over at least its first 30, 50, 70, 100, 150, 200, 250, or 300 nucleotides; and/or
c. the open reading frame consists of a set of codons of which at least 95%, 96%, 97%, 98%, 99%, 99.5%, or 100% of the codons are codons listed in Table 4, the low A set of Table 5, or the low A/U set of Table 5; and/or
d. the open reading frame has an adenine content ranging from its minimum adenine content to 123% of the minimum adenine content; and/or
e. the open reading frame has an adenine dinucleotide content ranging from its minimum adenine dinucleotide content to 150% of the minimum adenine dinucleotide content; and
(ii) a guide RNA or a vector encoding a guide RNA, wherein the guide RNA comprises a guide sequence selected from SEQ ID NOs: 5-72, 74-78, and 80-82.

Embodiment A2 is a method of modifying the TTR gene and/or inducing a double-stranded break (DSB) within the TTR gene, comprising delivering a composition to a cell, wherein the composition comprises:

(i) a nucleic acid comprising an open reading frame encoding an RNA-guided DNA binding agent, wherein:
a. the open reading frame comprises a sequence with at least 93% identity to SEQ ID NO:311; and/or
b. the open reading frame has at least 93% identity to SEQ ID NO: 311 over at least its first 50, 200, 250, or 300 nucleotides, or at least 95% identity to SEQ ID NO: 311 over at least its first 30, 50, 70, 100, 150, 200, 250, or 300 nucleotides; and/or
c. the open reading frame consists of a set of codons of which at least 95%, 96%, 97%, 98%, 99%, 99.5%, or 100% of the codons are codons listed in Table 4, the low A set of Table 5, or the low A/U set of Table 5; and/or
d. the open reading frame has an adenine content ranging from its minimum adenine content to 123% of the minimum adenine content; and/or
e. the open reading frame has an adenine dinucleotide content ranging from its minimum adenine dinucleotide content to 150% of the minimum adenine dinucleotide content; and
(ii) a guide RNA or a vector encoding a guide RNA, wherein the guide RNA comprises a guide sequence selected from SEQ ID NOs: 5-72, 74-78, and 80-82.

Embodiment A3 is a method of reducing TTR serum concentration, treating amyloidosis associated with TTR (ATTR), and/or reducing or preventing the accumulation of amyloids or amyloid fibrils comprising TTR in a subject, comprising administering a composition to a subject in need thereof, wherein the composition comprises:

(i) a nucleic acid comprising an open reading frame encoding an RNA-guided DNA binding agent, wherein:
a. the open reading frame comprises a sequence with at least 95% identity to SEQ ID NO:311; and/or
   b. the open reading frame has at least 95% identity to SEQ ID NO: 311 over at least its first 30, 50, 70, 100, 150, 200, 250, or 300 nucleotides; and/or
c. the open reading frame consists of a set of codons of which at least 95%, 96%, 97%, 98%, 99%, 99.5%, or 100% of the codons are codons listed in Table 4, the low A set of Table 5, or the low A/U set of Table 5; and/or
   d. the open reading frame has an adenine content ranging from its minimum adenine content to 150% of the minimum adenine content; and/or
   e. the open reading frame has an adenine dinucleotide content ranging from its minimum adenine dinucleotide content to 150% of the minimum adenine dinucleotide content; and
   (ii) a guide RNA or a vector encoding a guide RNA, wherein the guide RNA comprises a guide sequence selected from SEQ ID NOs: 5-72, 74-78, and 80-82, thereby reducing TTR serum concentration, treating amyloidosis associated with TTR (ATTR), and/or reducing or preventing the accumulation of amyloids or amyloid fibrils comprising TTR in the subject.

Embodiment A4 is the composition or method of any one of the preceding embodiments, wherein the guide RNA comprises a guide sequence selected from SEQ ID NOs: 5, 6, 7, 8, 9, 12, 13, 14, 15, 16, 17, 22, 23, 27, 29, 30, 35, 36, 37, 38, 55, 61, 63, 65, 66, 68, or 69.

Embodiment A5 is the composition of embodiment A1 or A4, for use in inducing a double-stranded break (DSB) within the TTR gene in a cell or subject.

Embodiment A6 is the composition of embodiment A1, A4, or A5 for use in modifying the TTR gene in a cell or subject.

Embodiment A7 is the composition of embodiment A1, A4, A5, or A6 for use in treating amyloidosis associated with TTR (ATTR) in a subject.

Embodiment A8 is the composition of embodiment A1, A4, A5, A6, or A7 for use in reducing TTR serum concentration in a subject.

Embodiment A9 is the composition of embodiment A1, A4, A5, A6, A7, or A8, for use in reducing or preventing the accumulation of amyloids or amyloid fibrils in a subject.

Embodiment A10 is the composition for use or method of any one of embodiments A2-A9, wherein the method comprises administering the composition by infusion for more than 30 minutes.

Embodiment A11 is the method or composition for use of embodiment A10, wherein the composition is administered by infusion for about 45-75 minutes, 75-105 minutes, 105-135 minutes, 135-165 minutes, 165-195 minutes, 195-225 minutes, 225-255 minutes, 255-285 minutes, 285-315 minutes, 315-345 minutes, or 345-375 minutes.

Embodiment A12 is the method or composition for use of embodiment A10 or 11, wherein the composition is administered by infusion for about 1.5-6 hours.

Embodiment A13 is the method or composition for use of embodiment A10, wherein the composition is administered by infusion for about 60 minutes, about 90 minutes, about 120 minutes, about 150 minutes, about 180 minutes, or about 240 minutes.

Embodiment A14 is the method or composition for use of embodiment A10, wherein the composition is administered by infusion for about 120 minutes.

Embodiment A15 is the method or composition for use of any one of embodiments A2-A14, wherein the composition reduces serum TTR levels.

Embodiment A16 is the method or composition for use of embodiment A15, wherein the serum TTR levels are reduced by at least 50% as compared to serum TTR levels before administration of the composition.

Embodiment A17 is the method or composition for use of embodiment A151, wherein the serum TTR levels are reduced by 50-60%, 60-70%, 70-80%, 80-90%, 90-95%, 95-98%, 98-99%, or 99-100% as compared to serum TTR levels before administration of the composition.

Embodiment Alb is the method or composition for use of any one of embodiments A2-17, wherein the composition results in editing of the TTR gene.

Embodiment A19 is the method or composition for use of embodiment A18, wherein the editing is calculated as a percentage of the population that is edited (percent editing).

Embodiment A20 is the method or composition for use of embodiment A19, wherein the percent editing is between 30 and 99% of the population.

Embodiment A21 is the method or composition for use of embodiment A19, wherein the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.

Embodiment A22 is the method or the composition for use of any one of embodiments A2-A21, wherein the composition reduces amyloid deposition in at least one tissue.

Embodiment A23 is the method or composition for use of embodiment A22, wherein the at least one tissue comprises one or more of stomach, colon, sciatic nerve, or dorsal root ganglion.

Embodiment A24 is the method or composition for use of embodiment A22 or 23, wherein amyloid deposition is measured 8 weeks after administration of the composition.

Embodiment A25 is the method or composition for use of any one of embodiments A22-A24, wherein amyloid deposition is compared to a negative control or a level measured before administration of the composition.

Embodiment A26 is the method or composition for use of any one of embodiments A22-A25, wherein amyloid deposition is measured in a biopsy sample and/or by immunostaining.

Embodiment A27 is the method or composition for use of any one of embodiments A22-A26, wherein amyloid deposition is reduced by between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the amyloid deposition seen in a negative control.

Embodiment A28 is the method or composition for use of any one of embodiments A22-A27, wherein amyloid deposition is reduced by between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the amyloid deposition seen before administration of the composition.

Embodiment A29 is the method or composition for use of any one of embodiments A2-A28, wherein the composition is administered or delivered at least two times.

Embodiment A30 is the method or composition for use of embodiment A29, wherein the composition is administered or delivered at least three times.

Embodiment A31 is the method or composition for use of embodiment A29, wherein the composition is administered or delivered at least four times.

Embodiment A32 is the method or composition for use of embodiment A29, wherein the composition is administered or delivered up to five, six, seven, eight, nine, or ten times.

Embodiment A33 is the method or composition for use of any one of embodiments A29-A32, wherein the administration or delivery occurs at an interval of 1, 2, 3, 4, 5, 6, 7, 8, 9, 10, 11, 12, 13, 14, or 15 days.

Embodiment A34 is the method or composition for use of any one of embodiments A29-A32, wherein the administration or delivery occurs at an interval of 1, 2, 3, 4, 5, 6, 7, 8, 9, 10, 11, 12, 13, 14, or 15 weeks.

Embodiment A35 is the method or composition for use of any one of embodiments A29-A32, wherein the administration or delivery occurs at an interval of 1, 2, 3, 4, 5, 6, 7, 8, 9, 10, 11, 12, 13, 14, or 15 months.

Embodiment A36 is the method or composition of any one of the preceding embodiments, wherein the guide RNA comprises a crRNA that comprises the guide sequence and further comprises a nucleotide sequence of SEQ ID NO: 126, wherein the nucleotides of SEQ ID NO: 126 follow the guide sequence at its 3′ end.

Embodiment A37 is the method or composition of any one of the preceding embodiments, wherein the guide RNA is a dual guide (dgRNA).

Embodiment A38 is the method or composition of embodiment A37, wherein the dual guide RNA comprises a crRNA comprising a nucleotide sequence of SEQ ID NO: 126, wherein the nucleotides of SEQ ID NO: 126 follow the guide sequence at its 3′ end, and a trRNA.

Embodiment A39 is the method or composition of any one of embodiments A1-A36, wherein the guide RNA is a single guide (sgRNA).

Embodiment A40 is the method or composition of embodiment A39, wherein the sgRNA comprises a guide sequence that has the pattern of SEQ ID NO: 3.

Embodiment A41 is the method or composition of embodiment A39, wherein the sgRNA comprises the sequence of SEQ ID NO: 3.

Embodiment A42 is the method or composition of any one of embodiments A39-A41, wherein the sgRNA comprises any one of the guide sequences of SEQ ID NOs: 5-72, 74-78, and 80-82 and the nucleotides of SEQ ID NO: 126.

Embodiment A43 is the method or composition of any one of embodiments A39-A42, wherein the sgRNA comprises a sequence that is at least 99%, 98%, 97%, 96%, 95%, 94%, 93%, 92%, 91%, or 90% identical to a sequence selected from SEQ ID Nos: 87-113, 115-120, and 122-124.

Embodiment A44 is the method or composition of embodiment A39, wherein the sgRNA comprises a sequence selected from SEQ ID Nos: 87-113, 115-120, and 122-124.

Embodiment A45 is the method or composition of any one of the preceding embodiments, wherein the guide RNA comprises at least one modification.

Embodiment A46 is the method or composition of embodiment A45, wherein the at least one modification includes a 2′-O-methyl (2′-O-Me) modified nucleotide.

Embodiment A47 is the method or composition of embodiment A45 or 46, wherein the at least one modification includes a phosphorothioate (PS) bond between nucleotides.

Embodiment A48 is the method or composition of any one of embodiments A45-A47, wherein the at least one modification includes a 2′-fluoro (2′-F) modified nucleotide.

Embodiment A49 is the method or composition of any one of embodiments A45-A48, wherein the at least one modification includes a modification at one or more of the first five nucleotides at the 5′ end.

Embodiment A50 is the method or composition of any one of embodiments A45-A49, wherein the at least one modification includes a modification at one or more of the last five nucleotides at the 3′ end.

Embodiment A51 is the method or composition of any one of embodiments A45-A50, wherein the at least one modification includes PS bonds between the first four nucleotides.

Embodiment A52 is the method or composition of any one of embodiments A45-A51, wherein the at least one modification includes PS bonds between the last four nucleotides.

Embodiment A53 is the method or composition of any one of embodiments A45-A52, wherein the at least one modification includes 2′-O-Me modified nucleotides at the first three nucleotides at the 5′ end.

Embodiment A54 is The method or composition of any one of embodiments A45-A53, wherein the at least one modification includes 2′-O-Me modified nucleotides at the last three nucleotides at the 3′ end.

Embodiment A55 is the method or composition of any one of embodiments A45-A54, wherein the guide RNA comprises the modified nucleotides of SEQ ID NO: 3.

Embodiment A56 is the method or composition of any one of embodiments A1-A55, wherein the composition further comprises a pharmaceutically acceptable excipient.

Embodiment A57 is the method or composition of any one of embodiments A1-A56, wherein the guide RNA and the nucleic acid comprising an open reading frame encoding an RNA-guided DNA binding agent are associated with a lipid nanoparticle (LNP).

Embodiment A58 is the method or composition of embodiment A57, wherein the LNP comprises a CCD lipid.

Embodiment A59 is the method or composition of embodiment A58, wherein the CCD lipid is Lipid A or Lipid B, optionally wherein the CCD lipid is lipid A.

Embodiment A60 is the method or composition of any one of embodiments A57-A59, wherein the LNP comprises a helper lipid.

Embodiment A61 is the method or composition of embodiment A60, wherein the helper lipid is cholesterol.

Embodiment A62 is the method or composition of any one of embodiments A57-A61, wherein the LNP comprises a stealth lipid (e.g., a PEG lipid).

Embodiment A63 is the method or composition of embodiment A62, wherein the stealth lipid is PEG2k-DMG.

Embodiment A64 is the method or composition of any one of embodiments A57-A63, wherein:

(i) the LNP comprises a lipid component and the lipid component comprises: about 50-60 mol-% amine lipid such as Lipid A, about 8-10 mol-% neutral lipid; and about 2.5-4 mol-% stealth lipid (e.g., a PEG lipid), wherein the remainder of the lipid component is helper lipid, and wherein the N/P ratio of the LNP composition is about 6;
(ii) the LNP comprises about 50-60 mol-% amine lipid such as Lipid A; about 27-39.5 mol-% helper lipid; about 8-10 mol-% neutral lipid; and about 2.5-4 mol-% stealth lipid (e.g., a PEG lipid), wherein the N/P ratio of the LNP composition is about 5-7 (e.g., about 6);
(iii) the LNP comprises a lipid component and the lipid component comprises: about 50-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 3-10;
(iv) the LNP comprises a lipid component and the lipid component comprises: about 40-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 6;
(v) the LNP comprises a lipid component and the lipid component comprises: about 50-60 mol-% amine lipid such as Lipid A; about 5-15 mol-% neutral lipid; and about 1.5-10 mol-% Stealth lipid (e.g., a PEG lipid), wherein the remainder of the lipid component is helper lipid, and wherein the N/P ratio of the LNP composition is about 6;
(vi) the LNP comprises a lipid component and the lipid component comprises: about 40-60 mol-% amine lipid such as Lipid A; about 0-10 mol-% neutral lipid; and about 1.5-10 mol-% Stealth lipid (e.g., a PEG lipid), wherein the remainder of the lipid component is helper lipid, and wherein the N/P ratio of the LNP composition is about 3-10;
(vii) the LNP comprises a lipid component and the lipid component comprises: about 40-60 mol-% amine lipid such as Lipid A; less than about 1 mol-% neutral lipid; and about 1.5-10 mol-% Stealth lipid (e.g., a PEG lipid), wherein the remainder of the lipid component is helper lipid, and wherein the N/P ratio of the LNP composition is about 3-10;
(viii) the LNP comprises a lipid component and the lipid component comprises: about 40-60 mol-% amine lipid such as Lipid A; and about 1.5-10 mol-% Stealth lipid (e.g., a PEG lipid), wherein the remainder of the lipid component is helper lipid, wherein the N/P ratio of the LNP composition is about 3-10, and wherein the LNP composition is essentially free of or free of neutral phospholipid; or
(ix) the LNP comprises a lipid component and the lipid component comprises: about 50-60 mol-% amine lipid such as Lipid A; about 8-10 mol-% neutral lipid; and about 2.5-4 mol-% Stealth lipid (e.g., a PEG lipid), wherein the remainder of the lipid component is helper lipid, and wherein the N/P ratio of the LNP composition is about 3-7.

Embodiment A64a is the method or composition of embodiment A64, wherein the mol-% PEG lipid is about 3.

Embodiment A64b is the method or composition of embodiment A64 or A64a, wherein the mol-% amine lipid is about 50.

Embodiment A64c is the method or composition of any one of embodiments A64-A64b, wherein the mol-% amine lipid is about 55.

Embodiment A64d is the method or composition of any one of embodiments A64-A64c, wherein the mol-% amine lipid is ±3 mol-%.

Embodiment A64e is the method or composition of any one of embodiments A64-A64d, wherein the mol-% amine lipid is ±2 mol-%.

Embodiment A64f is the method or composition of any one of embodiments A64-A64e, wherein the mol-% amine lipid is 47-53 mol-%.

Embodiment A64g is the method or composition of any one of embodiments A64-A64f, wherein the mol-% amine lipid is 48-53 mol-%.

Embodiment A64h is the method or composition of any one of embodiments A64-A64g, wherein the mol-% amine lipid is 53-57 mol-%.

Embodiment A64i is the method or composition of any one of embodiments A64-A64h, wherein the N/P ratio is 6±1.

Embodiment A64j is the method or composition of any one of embodiments A64-A64i, wherein the N/P ratio is 6±0.5.

Embodiment A64k is the method or composition of any one of embodiments A64-A64j, wherein the amine lipid is Lipid A.

Embodiment A64l is the method or composition of any one of embodiments A64-A64l, wherein the amine lipid is an analog of Lipid A.

Embodiment A64m is the method or composition of embodiment A64l, wherein the analog is an acetal analog.

Embodiment A64n is the method or composition of embodiment A64m, wherein the acetal analog is a C4-C12 acetal analog.

Embodiment A64o is the method or composition of embodiment A64m, wherein the acetal analog is a C5-C12 acetal analog.

Embodiment A64p is the method or composition of embodiment A64m, wherein the acetal analog is a C5-C10 acetal analog.

Embodiment A64q is the method or composition of embodiment A64m, wherein the acetal analog is chosen from a C4, C5, C6, C7, C9, C10, C11, and C12 analog.

Embodiment A64r is the method or composition of any one of embodiments A64-A64q, wherein the helper lipid is cholesterol.

Embodiment A64s is the method or composition of any one of embodiments A64-A64r, wherein the neutral lipid is DSPC.

Embodiment A64t is the method or composition of any one of embodiments A64-A64s, wherein the neutral lipid is DPPC.

Embodiment A64u is the method or composition of any one of embodiments A64-A64t, wherein the PEG lipid comprises dimyristoylglycerol (DMG).

Embodiment A64v is the method or composition of any one of embodiments A64-A64u, wherein the PEG lipid comprises a PEG-2k.

Embodiment A64w is the method or composition of any one of embodiments A64-A64v, wherein the PEG lipid is a PEG-DMG.

Embodiment A64x is the method or composition of embodiment A64w, wherein the PEG-DMG is a PEG2k-DMG.

Embodiment A64y is the method or composition of any one of embodiments A64-A64x, wherein the LNP composition is essentially free of neutral lipid.

Embodiment A64z is the method or composition of embodiment A64y, wherein the neutral lipid is a phospholipid.

Embodiment A65 is the method or composition of any one of embodiments A57-A64z, wherein the LNP comprises a neutral lipid, optionally wherein the neutral lipid is DSPC.

Embodiment A66 is the method or composition of any one of embodiments A64-A65, wherein the amine lipid is present at about 50 mol-%.

Embodiment A67 is the method or composition of any one of embodiments A64-A66, wherein the neutral lipid is present at about 9 mol-%.

Embodiment A68 is the method or composition of any one of embodiments A62-A67, wherein the stealth lipid is present at about 3 mol-%.

Embodiment A69 is the method or composition of any one of embodiments A60-A68, wherein the helper lipid is present at about 38 mol-%.

Embodiment A70 is the method or composition of any one of the preceding embodiments, wherein the LNP has an N/P ratio of about 6.

Embodiment A71 is the method or composition of embodiment A70, wherein the LNP comprises a lipid component and the lipid component comprises: about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the LNP composition is about 6.

Embodiment A72 is the method or composition of any one of embodiments A64-A71, wherein the amine lipid is Lipid A.

Embodiment A73 is the method or composition of any one of embodiments A64-A72, wherein the neutral lipid is DSPC.

Embodiment A74 is the method or composition of any one of embodiments A62-A73, wherein the stealth lipid is PEG2k-DMG.

Embodiment A75 is the method or composition of any one of embodiments A60-A74, wherein the helper lipid is cholesterol.

Embodiment A76 is the method or composition of any one of embodiments A70, wherein the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

Embodiment A77 is the method or composition of any one of the preceding embodiments, wherein the RNA-guided DNA binding agent is a Cas cleavase.

Embodiment A78 is the method or composition of embodiment A77, wherein the RNA-guided DNA binding agent is Cas9.

Embodiment A79 is the method or composition of any one of the preceding embodiments, wherein the RNA-guided DNA binding agent is modified.

Embodiment A80 is the method or composition of embodiment A79, wherein the modified RNA-guided DNA binding agent comprises a nuclear localization signal (NLS).

Embodiment A81 is the method or composition of any one of the preceding embodiments, wherein the RNA-guided DNA binding agent is a Cas from a Type-II CRISPR/Cas system.

Embodiment A82 is the method or composition of any one of the preceding embodiments, wherein the composition is a pharmaceutical formulation and further comprises a pharmaceutically acceptable carrier.

Embodiment A83 is the method or composition for use of any one of embodiments A2-A82, wherein the composition reduces or prevents amyloids or amyloid fibrils comprising TTR.

Embodiment A84 is the method or composition for use of embodiment A83, wherein the amyloids or amyloid fibrils are in the nerves, heart, or gastrointestinal track.

Embodiment A85 is the method or composition for use of any one of embodiments A2-A84, wherein non-homologous ending joining (NHEJ) leads to a mutation during repair of a DSB in the TTR gene.

Embodiment A86 is the method or composition for use of embodiment A85, wherein NHEJ leads to a deletion or insertion of a nucleotide(s) during repair of a DSB in the TTR gene.

Embodiment A87 is the method or composition for use of embodiment A86, wherein the deletion or insertion of a nucleotide(s) induces a frame shift or nonsense mutation in the TTR gene.

Embodiment A88 is the method or composition for use of embodiment A86, wherein a frame shift or nonsense mutation is induced in the TTR gene of at least 50% of liver cells.

Embodiment A89 is the method or composition for use of embodiment A88, wherein a frame shift or nonsense mutation is induced in the TTR gene of 50%-60%, 60%-70%, 70% or 80%, 80%-90%, 90-95%, 95%-99%, or 99%-100% of liver cells.

Embodiment A90 is the method or composition for use of any one of embodiments A86-A89, wherein a deletion or insertion of a nucleotide(s) occurs in the TTR gene at least 50-fold or more than in off-target sites.

Embodiment A91 is the method or composition for use of embodiment A90, wherein the deletion or insertion of a nucleotide(s) occurs in the TTR gene 50-fold to 150-fold, 150-fold to 500-fold, 500-fold to 1500-fold, 1500-fold to 5000-fold, 5000-fold to 15000-fold, 15000-fold to 30000-fold, or 30000-fold to 60000-fold more than in off-target sites.

Embodiment A92 is the method or composition for use of any one of embodiments A86-A91, wherein the deletion or insertion of a nucleotide(s) occurs at less than or equal to 3, 2, 1, or 0 off-target site(s) in primary human hepatocytes, optionally wherein the off-target site(s) does (do) not occur in a protein coding region in the genome of the primary human hepatocytes.

Embodiment A93 is the method or composition for use of embodiment A92, wherein the deletion or insertion of a nucleotide(s) occurs at a number of off-target sites in primary human hepatocytes that is less than the number of off-target sites at which a deletion or insertion of a nucleotide(s) occurs in Cas9-overexpressing cells, optionally wherein the off-target site(s) does (do) not occur in a protein coding region in the genome of the primary human hepatocytes.

Embodiment A94 is the method or composition for use of embodiment A93, wherein the Cas9-overexpressing cells are HEK293 cells stably expressing Cas9.

Embodiment A95 is the method or composition for use of any one of embodiments A92-A94, wherein the number of off-target sites in primary human hepatocytes is determined by analyzing genomic DNA from primary human hepatocytes transfected in vitro with Cas9 mRNA and the guide RNA, optionally wherein the off-target site(s) does (do) not occur in a protein coding region in the genome of the primary human hepatocytes.

Embodiment A96 is the method or composition for use of any one of embodiments A92-A94, wherein the number of off-target sites in primary human hepatocytes is determined by an oligonucleotide insertion assay comprising analyzing genomic DNA from primary human hepatocytes transfected in vitro with Cas9 mRNA, the guide RNA, and a donor oligonucleotide, optionally wherein the off-target site(s) does (do) not occur in a protein coding region in the genome of the primary human hepatocytes.

Embodiment A97 is the method or composition of any one of embodiments A1-A36 or A39-A96, wherein the sequence of the guide RNA is:
a) SEQ ID NO: 92 or 104;
b) SEQ ID NO: 87, 89, 96, or 113;
c) SEQ ID NO: 100, 102, 106, 111, or 112; or
d) SEQ ID NO: 88, 90, 91, 93, 94, 95, 97, 101, 103, 108, or 109.

Embodiment A98 is the method or composition of embodiment A97, wherein the guide RNA does not produce indels at off-target site(s) that occur in a protein coding region in the genome of primary human hepatocytes.

Embodiment A99 is the method or composition for use of any one of embodiments A2-98, wherein administering the composition reduces levels of TTR in the subject.

Embodiment A100 is the method or composition for use of embodiment A99, wherein the levels of TTR are reduced by at least 50%.

Embodiment A101 is the method or composition for use of embodiment A100, wherein the levels of TTR are reduced by 50%-60%, 60%-70%, 70% or 80%, 80%-90%, 90-95%, 95%-99%, or 99%-100%.

Embodiment A102 is the method or composition for use of embodiment A100 or A101, wherein the levels of TTR are measured in serum, plasma, blood, cerebral spinal fluid, or sputum.

Embodiment A103 is the method or composition for use of embodiment A100 or A101, wherein the levels of TTR are measured in liver, choroid plexus, and/or retina.

Embodiment A104 is the method or composition for use of any one of embodiments A99-A103, wherein the levels of TTR are measured via enzyme-linked immunosorbent assay (ELISA).

Embodiment A105 is the method or composition for use of any one of embodiments A2-A104, wherein the subject has ATTR.

Embodiment A106 is the method or composition for use of any one of embodiments A2-A105, wherein the subject is human.

Embodiment A107 is the method or composition for use of embodiment A105 or 106, wherein the subject has ATTRwt.

Embodiment A108 is the method or composition for use of embodiment A105 or 106, wherein the subject has hereditary ATTR.

Embodiment A109 is the method or composition for use of any one of embodiments A2-A106 or A108, wherein the subject has a family history of ATTR.

Embodiment A110 is the method or composition for use of any one of embodiments A2-A106 or A108-A109, wherein the subject has familial amyloid polyneuropathy.

Embodiment A111 is the method or composition for use of any one of embodiments A2-A110, wherein the subject has only or predominantly nerve symptoms of ATTR.

Embodiment A112 is the method or composition for use of any one of embodiments A2-A111, wherein the subject has familial amyloid cardiomyopathy.

Embodiment A113 is the method or composition for use of any one of embodiments A2-A110 or 112, wherein the subject has only or predominantly cardiac symptoms of ATTR.

Embodiment A114 is the method or composition for use of any one of embodiments A2-A113, wherein the subject expresses TTR having a V30 mutation.

Embodiment A115 is the method or composition for use of embodiment A114, wherein the V30 mutation is V30A, V30G, V30L, or V30M.

Embodiment A116 is the method or composition for use of embodiment A any one of embodiments A2-A113, wherein the subject expresses TTR having a T60 mutation.

Embodiment A117 is the method or composition for use of embodiment A116, wherein the T60 mutation is T60A.

Embodiment A118 is the method or composition for use of embodiment A any one of embodiments A2-A113, wherein the subject expresses TTR having a V122 mutation.

Embodiment A119 is the method or composition for use of embodiment A118, wherein the V122 mutation is V122A, V122I, or V122(−).

Embodiment A120 is the method or composition for use of any one of embodiments A2-A113, wherein the subject expresses wild-type TTR.

Embodiment A121 is the method or composition for use of any one of embodiments A2-A107, or A120, wherein the subject does not express TTR having a V30, T60, or V122 mutation.

Embodiment A122 is the method or composition for use of any one of embodiments A2-A107, or A120-A121, wherein the subject does not express TTR having a pathological mutation.

Embodiment A123 is the method or composition for use of embodiment A122, wherein the subject is homozygous for wild-type TTR.

Embodiment A124 is the method or composition for use of any one of embodiments A2-A123, wherein after administration the subject has an improvement, stabilization, or slowing of change in symptoms of sensorimotor neuropathy.

Embodiment A125 is the method or composition for use of embodiment A124, wherein the improvement, stabilization, or slowing of change in sensory neuropathy is measured using electromyogram, nerve conduction tests, or patient-reported outcomes.

Embodiment A126 is the method or composition for use of any one of embodiments A2-A125, wherein the subject has an improvement, stabilization, or slowing of change in symptoms of congestive heart failure.

Embodiment A127 is the method or composition for use of embodiment A126, wherein the improvement, stabilization, or slowing of change in congestive heart failure is measured using cardiac biomarker tests, lung function tests, chest x-rays, or electrocardiography.

Embodiment A128 is the method or composition for use of any one of embodiments A2-A127, wherein the composition or pharmaceutical formulation is administered via a viral vector.

Embodiment A129 is the method or composition for use of any one of embodiments A2-A127, wherein the composition or pharmaceutical formulation is administered via lipid nanoparticles.

Embodiment A130 is the method or composition for use of any one of embodiments A2-A129, wherein the subject is tested for specific mutations in the TTR gene before administering the composition or formulation.

Embodiment A131 is the method or composition of any one of the preceding embodiments, wherein the sequence selected from SEQ ID NOs: 5-72, 74-78, and 80-82 is SEQ ID NO: 5, 6, 9, 13, 14, 15, 16, 17, 22, 23, 27, 30, 35, 36, 37, 38, 55, 63, 65, 66, 68, or 69.

Embodiment A132 is the method or composition of any one of embodiments A1-A130, wherein the sequence selected from SEQ ID NOs: 5-72, 74-78, and 80-82 is SEQ ID NO: 5. Embodiment A133 is the method or composition of any one of embodiments A1-A130, wherein the sequence selected from SEQ ID NOs: 5-72, 74-78, and 80-82 is SEQ ID NO: 6. Embodiment A134 is the method or composition of any one of embodiments A1-A130, wherein the sequence selected from SEQ ID NOs: 5-72, 74-78, and 80-82 is SEQ ID NO: 7. Embodiment A135 is the method or composition of any one of embodiments A1-A130, wherein the sequence selected from SEQ ID NOs: 5-72, 74-78, and 80-82 is SEQ ID NO: 8. Embodiment A136 is the method or composition of any one of embodiments A1-A130, wherein the sequence selected from SEQ ID NOs: 5-72, 74-78, and 80-82 is SEQ ID NO: 9. Embodiment A137 is the method or composition of any one of embodiments A1-A130, wherein the sequence selected from SEQ ID NOs: 5-72, 74-78, and 80-82 is SEQ ID NO: 10. Embodiment A138 is the method or composition of any one of embodiments A1-A130, wherein the sequence selected from SEQ ID NOs: 5-72, 74-78, and 80-82 is SEQ ID NO: 11. Embodiment A139 is the method or composition of any one of embodiments A1-A130, wherein the sequence selected from SEQ ID NOs: 5-72, 74-78, and 80-82 is SEQ ID NO: 12. Embodiment A140 is the method or composition of any one of embodiments A1-A130, wherein the sequence selected from SEQ ID NOs: 5-72, 74-78, and 80-82 is SEQ ID NO: 13. Embodiment A141 is the method or composition of any one of embodiments A1-A130, wherein the sequence selected from SEQ ID NOs: 5-72, 74-78, and 80-82 is SEQ ID NO: 14. Embodiment A142 is the method or composition of any one of embodiments A1-A130, wherein the sequence selected from SEQ ID NOs: 5-72, 74-78, and 80-82 is SEQ ID NO: 15. Embodiment A143 is the method or composition of any one of embodiments A1-A130, wherein the sequence selected from SEQ ID NOs: 5-72, 74-78, and 80-82 is SEQ ID NO: 16. Embodiment A144 is the method or composition of any one of embodiments A1-A130, wherein the sequence selected from SEQ ID NOs: 5-72, 74-78, and 80-82 is SEQ ID NO: 17. Embodiment A145 is the method or composition of any one of embodiments A1-A130, wherein the sequence selected from SEQ ID NOs: 5-72, 74-78, and 80-82 is SEQ ID NO: 18. Embodiment A146 is the method or composition of any one of embodiments A1-A130, wherein the sequence selected from SEQ ID NOs: 5-72, 74-78, and 80-82 is SEQ ID NO: 19. Embodiment A147 is the method or composition of any one of embodiments A1-A130, wherein the sequence selected from SEQ ID NOs: 5-72, 74-78, and 80-82 is SEQ ID NO: 20. Embodiment A148 is the method or composition of any one of embodiments A1-A130, wherein the sequence selected from SEQ ID NOs: 5-72, 74-78, and 80-82 is SEQ ID NO: 21. Embodiment A149 is the method or composition of any one of embodiments A1-A130, wherein the sequence selected from SEQ ID NOs: 5-72, 74-78, and 80-82 is SEQ ID NO: 22. Embodiment A150 is the method or composition of any one of embodiments A1-A130, wherein the sequence selected from SEQ ID NOs: 5-72, 74-78, and 80-82 is SEQ ID NO: 23. Embodiment A151 is the method or composition of any one of embodiments A1-A130, wherein the sequence selected from SEQ ID NOs: 5-72, 74-78, and 80-82 is SEQ ID NO: 24. Embodiment A152 is the method or composition of any one of embodiments A1-A130, wherein the sequence selected from SEQ ID NOs: 5-72, 74-78, and 80-82 is SEQ ID NO: 25. Embodiment A153 is the method or composition of any one of embodiments A1-A130, wherein the sequence selected from SEQ ID NOs: 5-72, 74-78, and 80-82 is SEQ ID NO: 26. Embodiment A154 is the method or composition of any one of embodiments A1-A130, wherein the sequence selected from SEQ ID NOs: 5-72, 74-78, and 80-82 is SEQ ID NO: 27. Embodiment A155 is the method or composition of any one of embodiments A1-A130, wherein the sequence selected from SEQ ID NOs: 5-72, 74-78, and 80-82 is SEQ ID NO: 28. Embodiment A156 is the method or composition of any one of embodiments A1-A130, wherein the sequence selected from SEQ ID NOs: 5-72, 74-78, and 80-82 is SEQ ID NO: 29. Embodiment A157 is the method or composition of any one of embodiments A1-A130, wherein the sequence selected from SEQ ID NOs: 5-72, 74-78, and 80-82 is SEQ ID NO: 30. Embodiment A158 is the method or composition of any one of embodiments A1-A130, wherein the sequence selected from SEQ ID NOs: 5-72, 74-78, and 80-82 is SEQ ID NO: 31. Embodiment A159 is the method or composition of any one of embodiments A1-A130, wherein the sequence selected from SEQ ID NOs: 5-72, 74-78, and 80-82 is SEQ ID NO: 32. Embodiment A160 is the method or composition of any one of embodiments A1-A130, wherein the sequence selected from SEQ ID NOs: 5-72, 74-78, and 80-82 is SEQ ID NO: 33. Embodiment A161 is the method or composition of any one of embodiments A1-A130, wherein the sequence selected from SEQ ID NOs: 5-72, 74-78, and 80-82 is SEQ ID NO: 34. Embodiment A162 is the method or composition of any one of embodiments A1-A130, wherein the sequence selected from SEQ ID NOs: 5-72, 74-78, and 80-82 is SEQ ID NO: 35. Embodiment A163 is the method or composition of any one of embodiments A1-A130, wherein the sequence selected from SEQ ID NOs: 5-72, 74-78, and 80-82 is SEQ ID NO: 36. Embodiment A164 is the method or composition of any one of embodiments A1-A130, wherein the sequence selected from SEQ ID NOs: 5-72, 74-78, and 80-82 is SEQ ID NO: 37. Embodiment A165 is the method or composition of any one of embodiments A1-A130, wherein the sequence selected from SEQ ID NOs: 5-72, 74-78, and 80-82 is SEQ ID NO: 38. Embodiment A166 is the method or composition of any one of embodiments A1-A130, wherein the sequence selected from SEQ ID NOs: 5-72, 74-78, and 80-82 is SEQ ID NO: 39. Embodiment A167 is the method or composition of any one of embodiments A1-A130, wherein the sequence selected from SEQ ID NOs: 5-72, 74-78, and 80-82 is SEQ ID NO: 40. Embodiment A168 is the method or composition of any one of embodiments A1-A130, wherein the sequence selected from SEQ ID NOs: 5-72, 74-78, and 80-82 is SEQ ID NO: 41. Embodiment A169 is the method or composition of any one of embodiments A1-A130, wherein the sequence selected from SEQ ID NOs: 5-72, 74-78, and 80-82 is SEQ ID NO: 42. Embodiment A170 is the method or composition of any one of embodiments A1-A130, wherein the sequence selected from SEQ ID NOs: 5-72, 74-78, and 80-82 is SEQ ID NO: 43. Embodiment A171 is the method or composition of any one of embodiments A1-A130, wherein the sequence selected from SEQ ID NOs: 5-72, 74-78, and 80-82 is SEQ ID NO: 44. Embodiment A172 is the method or composition of any one of embodiments A1-A130, wherein the sequence selected from SEQ ID NOs: 5-72, 74-78, and 80-82 is SEQ ID NO: 45. Embodiment A173 is the method or composition of any one of embodiments A1-A130, wherein the sequence selected from SEQ ID NOs: 5-72, 74-78, and 80-82 is SEQ ID NO: 46. Embodiment A174 is the method or composition of any one of embodiments A1-A130, wherein the sequence selected from SEQ ID NOs: 5-72, 74-78, and 80-82 is SEQ ID NO: 47. Embodiment A175 is the method or composition of any one of embodiments A1-A130, wherein the sequence selected from SEQ ID NOs: 5-72, 74-78, and 80-82 is SEQ ID NO: 48. Embodiment A176 is the method or composition of any one of embodiments A1-A130, wherein the sequence selected from SEQ ID NOs: 5-72, 74-78, and 80-82 is SEQ ID NO: 49. Embodiment A177 is the method or composition of any one of embodiments A1-A130, wherein the sequence selected from SEQ ID NOs: 5-72, 74-78, and 80-82 is SEQ ID NO: 50. Embodiment A178 is the method or composition of any one of embodiments A1-A130, wherein the sequence selected from SEQ ID NOs: 5-72, 74-78, and 80-82 is SEQ ID NO: 51. Embodiment A179 is the method or composition of any one of embodiments A1-A130, wherein the sequence selected from SEQ ID NOs: 5-72, 74-78, and 80-82 is SEQ ID NO: 52. Embodiment A180 is the method or composition of any one of embodiments A1-A130, wherein the sequence selected from SEQ ID NOs: 5-72, 74-78, and 80-82 is SEQ ID NO: 53. Embodiment A181 is the method or composition of any one of embodiments A1-A130, wherein the sequence selected from SEQ ID NOs: 5-72, 74-78, and 80-82 is SEQ ID NO: 54. Embodiment A182 is the method or composition of any one of embodiments A1-A130, wherein the sequence selected from SEQ ID NOs: 5-72, 74-78, and 80-82 is SEQ ID NO: 55. Embodiment A183 is the method or composition of any one of embodiments A1-A130, wherein the sequence selected from SEQ ID NOs: 5-72, 74-78, and 80-82 is SEQ ID NO: 56. Embodiment A184 is the method or composition of any one of embodiments A1-A130, wherein the sequence selected from SEQ ID NOs: 5-72, 74-78, and 80-82 is SEQ ID NO: 57. Embodiment A185 is the method or composition of any one of embodiments A1-A130, wherein the sequence selected from SEQ ID NOs: 5-72, 74-78, and 80-82 is SEQ ID NO: 58. Embodiment A186 is the method or composition of any one of embodiments A1-A130, wherein the sequence selected from SEQ ID NOs: 5-72, 74-78, and 80-82 is SEQ ID NO: 59. Embodiment A187 is the method or composition of any one of embodiments A1-A130, wherein the sequence selected from SEQ ID NOs: 5-72, 74-78, and 80-82 is SEQ ID NO: 60. Embodiment A188 is the method or composition of any one of embodiments A1-A130, wherein the sequence selected from SEQ ID NOs: 5-72, 74-78, and 80-82 is SEQ ID NO: 61. Embodiment A189 is the method or composition of any one of embodiments A1-A130, wherein the sequence selected from SEQ ID NOs: 5-72, 74-78, and 80-82 is SEQ ID NO: 62. Embodiment A190 is the method or composition of any one of embodiments A1-A130, wherein the sequence selected from SEQ ID NOs: 5-72, 74-78, and 80-82 is SEQ ID NO: 63. Embodiment A191 is the method or composition of any one of embodiments A1-A130, wherein the sequence selected from SEQ ID NOs: 5-72, 74-78, and 80-82 is SEQ ID NO: 64. Embodiment A192 is the method or composition of any one of embodiments A1-A130, wherein the sequence selected from SEQ ID NOs: 5-72, 74-78, and 80-82 is SEQ ID NO: 65. Embodiment A193 is the method or composition of any one of embodiments A1-A130, wherein the sequence selected from SEQ ID NOs: 5-72, 74-78, and 80-82 is SEQ ID NO: 66. Embodiment A194 is the method or composition of any one of embodiments A1-A130, wherein the sequence selected from SEQ ID NOs: 5-72, 74-78, and 80-82 is SEQ ID NO: 67. Embodiment A195 is the method or composition of any one of embodiments A1-A130, wherein the sequence selected from SEQ ID NOs: 5-72, 74-78, and 80-82 is SEQ ID NO: 68. Embodiment A196 is the method or composition of any one of embodiments A1-A130, wherein the sequence selected from SEQ ID NOs: 5-72, 74-78, and 80-82 is SEQ ID NO: 69. Embodiment A197 is the method or composition of any one of embodiments A1-A130, wherein the sequence selected from SEQ ID NOs: 5-72, 74-78, and 80-82 is SEQ ID NO: 70. Embodiment A198 is the method or composition of any one of embodiments A1-A130, wherein the sequence selected from SEQ ID NOs: 5-72, 74-78, and 80-82 is SEQ ID NO: 71. Embodiment A199 is the method or composition of any one of embodiments A1-A130, wherein the sequence selected from SEQ ID NOs: 5-72, 74-78, and 80-82 is SEQ ID NO: 72. Embodiment A200 is the method or composition of any one of embodiments A1-A130, wherein the sequence selected from SEQ ID NOs: 5-72, 74-78, and 80-82 is SEQ ID NO: 74. Embodiment A201 is the method or composition of any one of embodiments A1-A130, wherein the sequence selected from SEQ ID NOs: 5-72, 74-78, and 80-82 is SEQ ID NO: 75. Embodiment A202 is the method or composition of any one of embodiments A1-A130, wherein the sequence selected from SEQ ID NOs: 5-72, 74-78, and 80-82 is SEQ ID NO: 76. Embodiment A203 is the method or composition of any one of embodiments A1-A130, wherein the sequence selected from SEQ ID NOs: 5-72, 74-78, and 80-82 is SEQ ID NO: 77. Embodiment A204 is the method or composition of any one of embodiments A1-A130, wherein the sequence selected from SEQ ID NOs: 5-72, 74-78, and 80-82 is SEQ ID NO: 78. Embodiment A205 is the method or composition of any one of embodiments A1-A130, wherein the sequence selected from SEQ ID NOs: 5-72, 74-78, and 80-82 is SEQ ID NO: 80. Embodiment A206 is the method or composition of any one of embodiments A1-A130, wherein the sequence selected from SEQ ID NOs: 5-72, 74-78, and 80-82 is SEQ ID NO: 81. Embodiment A207 is the method or composition of any one of embodiments A1-A130, wherein the sequence selected from SEQ ID NOs: 5-72, 74-78, and 80-82 is SEQ ID NO: 82. Embodiment A208 is the composition or method of any one of the preceding embodiments, wherein the open reading frame has at least 95% identity to SEQ ID NO: 311 over at least its first 10%, 12%, 15%, 20%, 25%, 30%, or 35% of its sequence.

Embodiment A209 is the composition or method of any one of the preceding embodiments, wherein the open reading frame comprises a sequence with at least 94%, 95%, 96%, 97%, 98%, 99%, 99.5%, or 100% identity to SEQ ID NO: 311.

Embodiment A210 is the composition or method of any one of the preceding embodiments, wherein at least 95%, 96%, 97%, 98%, 99%, 99.5%, or 100% of the codons of the open reading frame are codons listed in Table 4, Table 5, or Table 7.

Embodiment A211 is the composition or method of embodiment A210, wherein the codons listed in Table 4, Table 5, or Table 7 are codons listed in Table 4.

Embodiment A212 is the composition or method of embodiment A210, wherein the codons listed in Table 4, Table 5, or Table 7 are codons of the Low U codon set of Table 5.

Embodiment A213 is the composition or method of embodiment A210, wherein the codons listed in Table 4, Table 5, or Table 7 are codons of the Low A codon set of Table 5.

Embodiment A214 is the composition or method of embodiment A210, wherein the codons listed in Table 4, Table 5, or Table 7 are codons of the Low A/U codon set of Table 5.

Embodiment A215 is the composition or method of embodiment A210, wherein the codons listed in Table 4, Table 5, or Table 7 are codons listed in Table 7.

Embodiment A216 is the composition or method of any one of the preceding embodiments, wherein the open reading frame has an adenine content ranging from its minimum adenine content to 101%, 102%, 103%, 105%, 110%, 115%, 120%, or 123% of the minimum adenine content.

Embodiment A217 is the composition or method of any one of the preceding embodiments, wherein the open reading frame has an adenine dinucleotide content ranging from its minimum adenine dinucleotide content to 101%, 102%, 103%, 105%, 110%, 115%, 120%, 125%, 130%, 135%, 140%, 145%, or 150% of the minimum adenine dinucleotide content.

Embodiment A218 is the composition or method of any one of the preceding embodiments, wherein the nucleic acid comprises a 5′ UTR with at least 90% identity to any one of SEQ ID NOs: 232, 234, 236, 238, 241, or 275-277.

Embodiment A219 is the composition or method of any one of the preceding embodiments, wherein the nucleic acid comprises a 3′ UTR with at least 90% identity to any one of SEQ ID NOs: 233, 235, 237, 239, or 240.

Embodiment A220 is the composition or method of any one of the preceding embodiments, wherein the nucleic acid comprises a 5′ UTR and a 3′ UTR from the same source.

Embodiment A221 is the composition or method of any one of the preceding embodiments, wherein the nucleic acid is an mRNA comprising a 5′ cap selected from Cap0, Cap1, and Cap2.

Embodiment A222 is the composition or method of any one of the preceding embodiments, wherein the open reading frame comprises a sequence with at least 95%, 96%, 97%, 98%, 99%, 99.5%, or 100% identity to SEQ ID NO: 377.

Embodiment A223 is the composition or method of any of the preceding embodiments, wherein the nucleic acid is an mRNA in which at least 10% of the uridine is substituted with a modified uridine.

Embodiment A224 is the composition or method of embodiment A223, wherein the modified uridine is one or more of N1-methyl-pseudouridine, pseudouridine, 5-methoxyuridine, or 5-iodouridine.

Embodiment A225 is the composition or method of embodiment A223, wherein the modified uridine is one or both of N1-methyl-pseudouridine or 5-methoxyuridine.

Embodiment A226 is the composition or method of embodiment A223, wherein the modified uridine is N1-methyl-pseudouridine.

Embodiment A227 is the composition or method of embodiment A223, wherein the modified uridine is 5-methoxyuridine.

Embodiment A228 is the composition or method of any one of embodiments A223-A227, wherein 15% to 45% of the uridine in the mRNA is substituted with the modified uridine.

Embodiment A229 is the composition or method of any one of embodiments A223-A228, wherein at least 20% or at least 30% of the uridine in the mRNA is substituted with the modified uridine.

Embodiment A230 is the composition or method of embodiment A229, wherein at least 80% or at least 90% of the uridine in the mRNA is substituted with the modified uridine.

Embodiment A231 is the composition or method of embodiment A229, wherein 100% of the uridine in the mRNA is substituted with the modified uridine.

Embodiment A232 is a use of a composition or formulation of any of embodiments A1 or A4-A231 for the preparation of a medicament for treating a human subject having ATTR.

Sequence Table
The following sequence table provides a listing of sequences disclosed herein. It is understood that if a DNA sequence (comprising
Ts) is referenced with respect to an RNA, then Ts should be replaced with Us (which may be modified or unmodified depending on the context),
and vice versa.

| Description | Sequence | SEQ ID No. |
|---|---|---|
| Cas9 | GGGTCCCGCAGTCGGCGTCCAGCGGCTCTGCTTGTTCGTGTGTGTGTCGTTGCAGGCCTTATTCGGATCCGCCACCATGGACAAGA | 1 |
| transcript | AGTACAGCATCGGACTGGACATCGGAACAAACAGCGTCGGATGGGCAGTCATCACAGACGAATACAAGGTCCCGAGCAAGAAGTTC | |
| with 5′ UTR | AAGGTCCTGGGAAACACAGACAGACACAGCATCAAGAAGAACCTGATCGGAGCACTGCTGTTCGACAGCGGAGAAACAGCAGAAGC | |
| of HSD, ORF | AACAAGACTGAAGAGAACAGCAAGAAGAAGATACACAAGAAGAAAGAACAGAATCTGCTACCTGCAGGAAATCTTCAGCAACGAAA | |
| corresponding | TGGCAAAGGTCGACGACAGCTTCTTCCACAGACTGGAAGAAAGCTTCCTGGTCGAAGAAGACAAGAAGCACGAAAGACACCCGATC | |
| to SEQ ID | TTCGGAAACATCGTCGACGAAGTCGCATACCACGAAAAGTACCCGACAATCTACCACCTGAGAAAGAAGCTGGTCGACAGCACAGA | |
| NO: 204, | CAAGGCAGACCTGAGACTGATCTACCTGGCACTGGCACACATGATCAAGTTCAGAGGACACTTCCTGATCGAAGGAGACCTGAACC | |
| Kozak | CGGACAACAGCGACGTCGACAAGCTGTTCATCCAGCTGGTCCAGACATACAACCAGCTGTTCGAAGAAAACCCGATCAACGCAAGC | |
| sequence, | GGAGTCGACGCAAAGGCAATCCTGAGCGCAAGACTGAGCAAGAGCAGAAGACTGGAAAACCTGATCGCACAGCTGCCGGGAGAAAA | |
| and 3′ UTR | GAAGAACGGACTGTTCGGAAACCTGATCGCACTGAGCCTGGGACTGACACCGAACTTCAAGAGCAACTTCGACCTGGCAGAAGACG | |
| of ALB | CAAAGCTGCAGCTGAGCAAGGACACATACGACGACGACCTGGACAACCTGCTGGCACAGATCGGAGACCAGTACGCAGACCTGTTC | |
| | CTGGCAGCAAAGAACCTGAGCGACGCAATCCTGCTGAGCGACATCCTGAGAGTCAACACAGAAATCACAAAGGCACCGCTGAGCGC | |
| | AAGCATGATCAAGAGATACGACGAACACCACCAGGACCTGACACTGCTGAAGGCACTGGTCAGACAGCAGCTGCCGGAAAAGTACA | |
| | AGGAAATCTTCTTCGACCAGAGCAAGAACGGATACGCAGGATACATCGACGGAGGAGCAAGCCAGGAAGAATTCTACAAGTTCATC | |
| | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCT | |
| | AGCTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 | GGGTCCCGCAGTCGGCGTCCAGCGGCTCTGCTTGTTCGTGTGTGTGTCGTTGCAGGCCTTATTCGGATCCATGCCTAAGAAAAAGC | 2 |
| transcript | GGAAGGTCGACGGGGATAAGAAGTACTCAATCGGGCTGGATATCGGAACTAATTCCGTGGGTTGGGCAGTGATCACGGATGAATAC | |
| comprising | AAAGTGCCGTCCAAGAAGTTCAAGGTCCTGGGGAACACCGATAGACACAGCATCAAGAAAAATCTCATCGGAGCCCTGCTGTTTGA | |
| Cas9 ORF | CTCCGGCGAAACCGCAGAAGCGACCCGGCTCAAACGTACCGCGAGGCGACGCTACACCCGGCGGAAGAATCGCATCTGCTATCTGC | |
| corresponding | AAGAGATCTTTTCGAACGAAATGGCAAAGGTCGACGACAGCTTCTTCCACCGCCTGGAAGAATCTTTCCTGGTGGAGGAGGACAAG | |
| to SEQ ID | AAGCATGAACGGCATCCTATCTTTGGAAACATCGTCGACGAAGTGGCGTACCACGAAAAGTACCCGACCATCTACCATCTGCGGAA | |
| NO: 205 | GAAGTTGGTTGACTCAACTGACAAGGCCGACCTCAGATTGATCTACTTGGCCCTCGCCCATATGATCAAATTCCGCGGACACTTCC | |
| using codons | TGATCGAAGGCGATCTGAACCCTGATAACTCCGACGTGGATAAGCTTTTCATTCAACTGGTGCAGACCTACAACCAACTGTTCGAA | |
| with | GAAAACCCAATCAATGCTAGCGGCGTCGATGCCAAGGCCATCCTGTCCGCCCGGCTGTCGAAGTCGCGGCGCCTCGAAAACCTGAT | |
| generally | CGCACAGCTGCCGGGAGAGAAAAAGAACGGACTTTTCGGCAACTTGATCGCTCTCTCACTGGGACTCACTCCCAATTTCAAGTCCA | |
| high | ATTTTGACCTGGCCGAGGACGCGAAGCTGCAACTCTCAAAGGACACCTACGACGACGACTTGGACAATTTGCTGGCACAAATTGGC | |
| expression | GATCAGTACGCGGATCTGTTCCTTGCCGCTAAGAACCTTTCGGACGCAATCTTGCTGTCCGATATCCTGCGCGTGAACACCGAAAT | |
| in humans | AACCAAAGCGCCGCTTAGCGCCTCGATGATTAAGCGGTACGACGAGCATCACCAGGATCTCACGCTGCTCAAAGCGCTCGTGAGAC | |
| | AGCAACTGCCTGAAAAGTACAAGGAGATCTTCTTCGACCAGTCCAAGAATGGGTACGCAGGGTACATCGATGGAGGCGCTAGCCAG | |
| | GAAGAGTTCTATAAGTTCATCAAGCCAATCCTGGAAAAGATGGACGGAACCGAAGAACTGCTGGTCAAGCTGAACAGGGAGGATCT | |
| | GCTCCGGAAACAGAGAACCTTTGACAACGGATCCATTCCCCACCAGATCCATCTGGGTGAGCTGCACGCCATCTTGCGGCGCCAGG | |
| | AGGACTTTTACCCATTCCTCAAGGACAACCGGGAAAAGATCGAGAAAATTCTGACGTTCCGCATCCCGTATTACGTGGGCCCACTG | |
| | GCGCGCGGCAATTCGCGCTTCGCGTGGATGACTAGAAAATCAGAGGAAACCATCACTCCTTGGAATTTCGAGGAAGTTGTGGATAA | |
| | GGGAGCTTCGGCACAAAGCTTCATCGAACGAATGACCAACTTCGACAAGAATCTCCCAAACGAGAAGGTGCTTCCTAAGCACAGCC | |
| | TCCTTTACGAATACTTCACTGTCTACAACGAACTGACTAAAGTGAAATACGTTACTGAAGGAATGAGGAAGCCGGCCTTTCTGTCC | |
| | GGAGAACAGAAGAAAGCAATTGTCGATCTGCTGTTCAAGACCAACCGCAAGGTGACCGTCAAGCAGCTTAAAGAGGACTACTTCAA | |
| | GAAGATCGAGTGTTTCGACTCAGTGGAAATCAGCGGGGTGGAGGACAGATTCAACGCTTCGCTGGGAACCTATCATGATCTCCTGA | |
| | AGATCATCAAGGACAAGGACTTCCTTGACAACGAGGAGAACGAGGACATCCTGGAAGATATCGTCCTGACCTTGACCCTTTTCGAG | |
| | GATCGCGAGATGATCGAGGAGAGGCTTAAGACCTACGCTCATCTCTTCGACGATAAGGTCATGAAACAACTCAAGCGCCGCCGGTA | |
| | CACTGGTTGGGGCCGCCTCTCCCGCAAGCTGATCAACGGTATTCGCGATAAACAGAGCGGTAAAACTATCCTGGATTTCCTCAAAT | |
| | CGGATGGCTTCGCTAATCGTAACTTCATGCAATTGATCCACGACGACAGCCTGACCTTTAAGGAGGACATCCAAAAAGCACAAGTG | |
| | TCCGGACAGGGAGACTCACTCCATGAACACATCGCGAATCTGGCCGGTTCGCCGGCGATTAAGAAGGGAATTCTGCAAACTGTGAA | |
| | GGTGGTCGACGAGCTGGTGAAGGTCATGGGACGGCACAAACCGGAGAATATCGTGATTGAAATGGCCCGAGAAAACCAGACTACCC | |
| | AGAAGGGCCAGAAAAACTCCCGCGAAAGGATGAAGCGGATCGAAGAAGGAATCAAGGAGCTGGGCAGCCAGATCCTGAAAGAGCAC | |
| | CCGGTGGAAAACACGCAGCTGCAGAACGAGAAGCTCTACCTGTACTATTTGCAAAATGGACGGGACATGTACGTGGACCAAGAGCT | |
| | GGACATCAATCGGTTGTCTGATTACGACGTGGACCACATCGTTCCACAGTCCTTTCTGAAGGATGACTCGATCGATAACAAGGTGT | |
| | TGACTCGCAGCGACAAGAACAGAGGGAAGTCAGATAATGTGCCATCGGAGGAGGTCGTGAAGAAGATGAAGAATTACTGGCGGCAG | |
| | CTCCTGAATGCGAAGCTGATTACCCAGAGAAAGTTTGACAATCTCACTAAAGCCGAGCGCGGCGGACTCTCAGAGCTGGATAAGGC | |
| | TGGATTCATCAAACGGCAGCTGGTCGAGACTCGGCAGATTACCAAGCACGTGGCGCAGATCTTGGACTCCCGCATGAACACTAAAT | |
| | ACGACGAGAACGATAAGCTCATCCGGGAAGTGAAGGTGATTACCCTGAAAAGCAAACTTGTGTCGGACTTTCGGAAGGACTTTCAG | |
| | TTTTACAAAGTGAGAGAAATCAACAACTACCATCACGCGCATGACGCATACCTCAACGCTGTGGTCGGTACCGCCCTGATCAAAAA | |
| | GTACCCTAAACTTGAATCGGAGTTTGTGTACGGAGACTACAAGGTCTACGACGTGAGGAAGATGATAGCCAAGTCCGAACAGGAAA | |
| | TCGGGAAAGCAACTGCGAAATACTTCTTTTACTCAAACATCATGAACTTTTTCAAGACTGAAATTACGCTGGCCAATGGAGAAATC | |
| | AGGAAGAGGCCACTGATCGAAACTAACGGAGAAACGGGCGAAATCGTGTGGGACAAGGGCAGGGACTTCGCAACTGTTCGCAAAGT | |
| | GCTCTCTATGCCGCAAGTCAATATTGTGAAGAAAACCGAAGTGCAAACCGGCGGATTTTCAAAGGAATCGATCCTCCCAAAGAGAA | |
| | ATAGCGACAAGCTCATTGCACGCAAGAAAGACTGGGACCCGAAGAAGTACGGAGGATTCGATTCGCCGACTGTCGCATACTCCGTC | |
| | CTCGTGGTGGCCAAGGTGGAGAAGGGAAAGAGCAAAAAGCTCAAATCCGTCAAAGAGCTGCTGGGGATTACCATCATGGAACGATC | |
| | CTCGTTCGAGAAGAACCCGATTGATTTCCTCGAGGCGAAGGGTTACAAGGAGGTGAAGAAGGATCTGATCATCAAACTCCCCAAGT | |
| | ACTCACTGTTCGAACTGGAAAATGGTCGGAAGCGCATGCTGGCTTCGGCCGGAGAACTCCAAAAAGGAAATGAGCTGGCCTTGCCT | |
| | AGCAAGTACGTCAACTTCCTCTATCTTGCTTCGCACTACGAAAAACTCAAAGGGTCACCGGAAGATAACGAACAGAAGCAGCTTTT | |
| | CGTGGAGCAGCACAAGCATTATCTGGATGAAATCATCGAACAAATCTCCGAGTTTTCAAAGCGCGTGATCCTCGCCGACGCCAACC | |
| | TCGACAAAGTCCTGTCGGCCTACAATAAGCATAGAGATAAGCCGATCAGAGAACAGGCCGAGAACATTATCCACTTGTTCACCCTG | |
| | ACTAACCTGGGAGCCCCAGCCGCCTTCAAGTACTTCGATACTACTATCGATCGCAAAAGATACACGTCCACCAAGGAAGTTCTGGA | |
| | CGCGACCCTGATCCACCAAAGCATCACTGGACTCTACGAAACTAGGATCGATCTGTCGCAGCTGGGTGGCGATTGATAGTCTAGCC | |
| | ATCACATTTAAAAGCATCTCAGCCTACCATGAGAATAAGAGAAAGAAAATGAAGATCAATAGCTTATTCATCTCTTTTTCTTTTTC | |
| | GTTGGTGTAAAGCCAACACCCTGTCTAAAAAACATAAATTTCTTTAATCATTTTGCCTCTTTTCTCTGTGCTTCAATTAATAAAAA | |
| | ATGGAAAGAACCTCGAG | |
| modified | mN*mN*mN*NNNNNNNNNNNNNNNNNGUUUUAGAmGmCmUmAmGmAmAmAmUmAmGmCAAGUUAAAAUAAGGCUAGUCCGUUAUCA | 3 |
| sgRNA | mAmCmUmUmGGmUmGmGmCmAmCmCmGmAmGmUmCmGmGmUmGmCmU*mU*mU*mU | |
| sequence | | |
| (“N” may be | | |
| any natural | | |
| or non- | | |
| natural | | |
| nucleotide) | | |
| 30/30/39 | AAAAAAAAAAAAAAAAAAAAAAAAAAAAAAGCGAAAAAAAAAAAAAAAAAAAAAAAAAAAAAACCGAAAAAAAAAAAAAAAAAAAA | 4 |
| poly-A | | |
| sequence | | |
| CR003335 | CUGCUCCUCCUCUGCCUUGC | 5 |
| gRNA | | |
| targeting | | |
| Human TTR | | |
| (Exon 1) | | |
| CR003336 | CCUCCUCUGCCUUGCUGGAC | 6 |
| gRNA | | |
| targeting | | |
| Human TTR | | |
| (Exon 1) | | |
| CR003337 | CCAGUCCAGCAAGGCAGAGG | 7 |
| gRNA | | |
| targeting | | |
| Human TTR | | |
| (Exon 1) | | |
| CR003338 | AUACCAGUCCAGCAAGGCAG | 8 |
| gRNA | | |
| targeting | | |
| Human TTR | | |
| (Exon 1) | | |
| CR003339 | ACACAAAUACCAGUCCAGCA | 9 |
| gRNA | | |
| targeting | | |
| Human TTR | | |
| (Exon 1) | | |
| CR003340 | UGGACUGGUAUUUGUGUCUG | 10 |
| gRNA | | |
| targeting | | |
| Human TTR | | |
| (Exon 1) | | |
| CR003341 | CUGGUAUUUGUGUCUGAGGC | 11 |
| gRNA | | |
| targeting | | |
| Human TTR | | |
| (Exon 1) | | |
| CR003342 | CUUCUCUACACCCAGGGCAC | 12 |
| gRNA | | |
| targeting | | |
| Human TTR | | |
| (Exon 2) | | |
| CR003343 | CAGAGGACACUUGGAUUCAC | 13 |
| gRNA | | |
| targeting | | |
| Human TTR | | |
| (Exon 2) | | |
| CR003344 | UUUGACCAUCAGAGGACACU | 14 |
| gRNA | | |
| targeting | | |
| Human TTR | | |
| (Exon 2) | | |
| CR003345 | UCUAGAACUUUGACCAUCAG | 15 |
| gRNA | | |
| targeting | | |
| Human TTR | | |
| (Exon 2) | | |
| CR003346 | AAAGUUCUAGAUGCUGUCCG | 16 |
| gRNA | | |
| targeting | | |
| Human TTR | | |
| (Exon 2) | | |
| CR003347 | CAUUGAUGGCAGGACUGCCU | 17 |
| gRNA | | |
| targeting | | |
| Human TTR | | |
| (Exon 2) | | |
| CR003348 | AGGCAGUCCUGCCAUCAAUG | 18 |
| gRNA | | |
| targeting | | |
| Human TTR | | |
| (Exon 2) | | |
| CR003349 | UGCACGGCCACAUUGAUGGC | 19 |
| gRNA | | |
| targeting | | |
| Human TTR | | |
| (Exon 2) | | |
| CR003350 | CACAUGCACGGCCACAUUGA | 20 |
| gRNA | | |
| targeting | | |
| Human TTR | | |
| (Exon 2) | | |
| CR003351 | AGCCUUUCUGAACACAUGCA | 21 |
| gRNA | | |
| targeting | | |
| Human TTR | | |
| (Exon 2) | | |
| CR003352 | GAAAGGCUGCUGAUGACACC | 22 |
| gRNA | | |
| targeting | | |
| Human TTR | | |
| (Exon 2) | | |
| CR003353 | AAAGGCUGCUGAUGACACCU | 23 |
| gRNA | | |
| targeting | | |
| Human TTR | | |
| (Exon 2) | | |
| CR003354 | ACCUGGGAGCCAUUUGCCUC | 24 |
| gRNA | | |
| targeting | | |
| Human TTR | | |
| (Exon 2) | | |
| CR003355 | CCCAGAGGCAAAUGGCUCCC | 25 |
| gRNA | | |
| targeting | | |
| Human TTR | | |
| (Exon 2) | | |
| CR003356 | GCAACUUACCCAGAGGCAAA | 26 |
| gRNA | | |
| targeting | | |
| Human TTR | | |
| (Exon 2) | | |
| CR003357 | UUCUUUGGCAACUUACCCAG | 27 |
| gRNA | | |
| targeting | | |
| Human TTR | | |
| (Exon 2) | | |
| CR003358 | AUGCAGCUCUCCAGACUCAC | 28 |
| gRNA | | |
| targeting | | |
| Human TTR | | |
| (Exon 3) | | |
| CR003359 | AGUGAGUCUGGAGAGCUGCA | 29 |
| gRNA | | |
| targeting | | |
| Human TTR | | |
| (Exon 3) | | |
| CR003360 | GUGAGUCUGGAGAGCUGCAU | 30 |
| gRNA | | |
| targeting | | |
| Human TTR | | |
| (Exon 3) | | |
| CR003361 | GCUGCAUGGGCUCACAACUG | 31 |
| gRNA | | |
| targeting | | |
| Human TTR | | |
| (Exon 3) | | |
| CR003362 | GCAUGGGCUCACAACUGAGG | 32 |
| gRNA | | |
| targeting | | |
| Human TTR | | |
| (Exon 3) | | |
| CR003363 | ACUGAGGAGGAAUUUGUAGA | 33 |
| gRNA | | |
| targeting | | |
| Human TTR | | |
| (Exon 3) | | |
| CR003364 | CUGAGGAGGAAUUUGUAGAA | 34 |
| gRNA | | |
| targeting | | |
| Human TTR | | |
| (Exon 3) | | |
| CR003365 | UGUAGAAGGGAUAUACAAAG | 35 |
| gRNA | | |
| targeting | | |
| Human TTR | | |
| (Exon 3) | | |
| CR003366 | AAAUAGACACCAAAUCUUAC | 36 |
| gRNA | | |
| targeting | | |
| Human TTR | | |
| (Exon 3) | | |
| CR003367 | AGACACCAAAUCUUACUGGA | 37 |
| gRNA | | |
| targeting | | |
| Human TTR | | |
| (Exon 3) | | |
| CR003368 | AAGUGCCUUCCAGUAAGAUU | 38 |
| gRNA | | |
| targeting | | |
| Human TTR | | |
| (Exon 3) | | |
| CR003369 | CUCUGCAUGCUCAUGGAAUG | 39 |
| gRNA | | |
| targeting | | |
| Human TTR | | |
| (Exon 3) | | |
| CR003370 | CCUCUGCAUGCUCAUGGAAU | 40 |
| gRNA | | |
| targeting | | |
| Human TTR | | |
| (Exon 3) | | |
| CR003371 | ACCUCUGCAUGCUCAUGGAA | 41 |
| gRNA | | |
| targeting | | |
| Human TTR | | |
| (Exon 3) | | |
| CR003372 | UACUCACCUCUGCAUGCUCA | 42 |
| gRNA | | |
| targeting | | |
| Human TTR | | |
| (Exon 3) | | |
| CR003373 | GUAUUCACAGCCAACGACUC | 43 |
| gRNA | | |
| targeting | | |
| Human TTR | | |
| (Exon 4) | | |
| CR003374 | GCGGCGGGGGCCGGAGUCGU | 44 |
| gRNA | | |
| targeting | | |
| Human TTR | | |
| (Exon 4) | | |
| CR003375 | AAUGGUGUAGCGGCGGGGGC | 45 |
| gRNA | | |
| targeting | | |
| Human TTR | | |
| (Exon 4) | | |
| CR003376 | CGGCAAUGGUGUAGCGGCGG | 46 |
| gRNA | | |
| targeting | | |
| Human TTR | | |
| (Exon 4) | | |
| CR003377 | GCGGCAAUGGUGUAGCGGCG | 47 |
| gRNA | | |
| targeting | | |
| Human TTR | | |
| (Exon 4) | | |
| CR003378 | GGCGGCAAUGGUGUAGCGGC | 48 |
| gRNA | | |
| targeting | | |
| Human TTR | | |
| (Exon 4) | | |
| CR003379 | GGGCGGCAAUGGUGUAGCGG | 49 |
| gRNA | | |
| targeting | | |
| Human TTR | | |
| (Exon 4) | | |
| CR003380 | GCAGGGCGGCAAUGGUGUAG | 50 |
| gRNA | | |
| targeting | | |
| Human TTR | | |
| (Exon 4) | | |
| CR003381 | GGGGCUCAGCAGGGCGGCAA | 51 |
| gRNA | | |
| targeting | | |
| Human TTR | | |
| (Exon 4) | | |
| CR003382 | GGAGUAGGGGCUCAGCAGGG | 52 |
| gRNA | | |
| targeting | | |
| Human TTR | | |
| (Exon 4) | | |
| CR003383 | AUAGGAGUAGGGGCUCAGCA | 53 |
| gRNA | | |
| targeting | | |
| Human TTR | | |
| (Exon 4) | | |
| CR003384 | AAUAGGAGUAGGGGCUCAGC | 54 |
| gRNA | | |
| targeting | | |
| Human TTR | | |
| (Exon 4) | | |
| CR003385 | CCCCUACUCCUAUUCCACCA | 55 |
| gRNA | | |
| targeting | | |
| Human TTR | | |
| (Exon 4) | | |
| CR003386 | CCGUGGUGGAAUAGGAGUAG | 56 |
| gRNA | | |
| targeting | | |
| Human TTR | | |
| (Exon 4) | | |
| CR003387 | GCCGUGGUGGAAUAGGAGUA | 57 |
| gRNA | | |
| targeting | | |
| Human TTR | | |
| (Exon 4) | | |
| CR003388 | GACGACAGCCGUGGUGGAAU | 58 |
| gRNA | | |
| targeting | | |
| Human TTR | | |
| (Exon 4) | | |
| CR003389 | AUUGGUGACGACAGCCGUGG | 59 |
| gRNA | | |
| targeting | | |
| Human TTR | | |
| (Exon 4) | | |
| CR003390 | GGGAUUGGUGACGACAGCCG | 60 |
| gRNA | | |
| targeting | | |
| Human TTR | | |
| (Exon 4) | | |
| CR003391 | GGCUGUCGUCACCAAUCCCA | 61 |
| gRNA | | |
| targeting | | |
| Human TTR | | |
| (Exon 4) | | |
| CR003392 | AGUCCCUCAUUCCUUGGGAU | 62 |
| gRNA | | |
| targeting | | |
| Human TTR | | |
| (Exon 4) | | |
| CR005298 | UCCACUCAUUCUUGGCAGGA | 63 |
| gRNA | | |
| targeting | | |
| Human TTR | | |
| (Exon 1) | | |
| CR005299 | AGCCGUGGUGGAAUAGGAGU | 64 |
| gRNA | | |
| targeting | | |
| Human TTR | | |
| (Exon 4) | | |
| CR005300 | UCACAGAAACACUCACCGUA | 65 |
| gRNA | | |
| targeting | | |
| Human TTR | | |
| (Exon 1) | | |
| CR005301 | GUCACAGAAACACUCACCGU | 66 |
| gRNA | | |
| targeting | | |
| Human TTR | | |
| (Exon 1) | | |
| CR005302 | ACGUGUCUUCUCUACACCCA | 67 |
| gRNA | | |
| targeting | | |
| Human TTR | | |
| (Exon 2) | | |
| CR005303 | UGAAUCCAAGUGUCCUCUGA | 68 |
| gRNA | | |
| targeting | | |
| Human TTR | | |
| (Exon 2) | | |
| CR005304 | GGCCGUGCAUGUGUUCAGAA | 69 |
| gRNA | | |
| targeting | | |
| Human TTR | | |
| (Exon 2) | | |
| CR005305 | UAUAGGAAAACCAGUGAGUC | 70 |
| gRNA | | |
| targeting | | |
| Human TTR | | |
| (Exon 3) | | |
| CR005306 | AAAUCUUACUGGAAGGCACU | 71 |
| gRNA | | |
| targeting | | |
| Human TTR | | |
| (Exon 3) | | |
| CR005307 | UGUCUGUCUUCUCUCAUAGG | 72 |
| gRNA | | |
| targeting | | |
| Human TTR | | |
| (Exon 4) | | |
| CR000689 | ACACAAAUACCAGUCCAGCG | 73 |
| gRNA | | |
| targeting | | |
| Cyno TTR | | |
| CR005364 | AAAGGCUGCUGAUGAGACCU | 74 |
| gRNA | | |
| targeting | | |
| Cyno TTR | | |
| CR005365 | CAUUGACAGCAGGACUGCCU | 75 |
| gRNA | | |
| targeting | | |
| Cyno TTR | | |
| CR005366 | AUACCAGUCCAGCGAGGCAG | 76 |
| gRNA | | |
| targeting | | |
| Cyno TTR | | |
| CR005367 | CCAGUCCAGCGAGGCAGAGG | 77 |
| gRNA | | |
| targeting | | |
| Cyno TTR | | |
| CR005368 | CCUCCUCUGCCUCGCUGGAC | 78 |
| gRNA | | |
| targeting | | |
| Cyno TTR | | |
| CR005369 | AAAGUUCUAGAUGCCGUCCG | 79 |
| gRNA | | |
| targeting | | |
| Cyno TTR | | |
| CR005370 | ACUUGUCUUCUCUAUACCCA | 80 |
| gRNA | | |
| targeting | | |
| Cyno TTR | | |
| CR005371 | AAGUGACUUCCAGUAAGAUU | 81 |
| gRNA | | |
| targeting | | |
| Cyno TTR | | |
| CR005372 | AAAAGGCUGCUGAUGAGACC | 82 |
| gRNA | | |
| targeting | | |
| Cyno TTR | | |
| | Not Used | 83 |
| | Not Used | 84 |
| | Not Used | 85 |
| | Not Used | 86 |
| G000480 | mA*mA*mA*GGCUGCUGAUGACACCUGUUUUAGAmGmCmUmAmGmAmAmAmUmAmGmCAAGUUAAAAUAAGGCUAGUCCGUUAUCA | 87 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000481 | mU*mC*mU*AGAACUUUGACCAUCAGGUUUUAGAmGmCmUmAmGmAmAmAmUmAmGmCAAGUUAAAAUAAGGCUAGUCCGUUAUCA | 88 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000482 | mU*mG*mU*AGAAGGGAUAUACAAAGGUUUUAGAmGmCmUmAmGmAmAmAmUmAmGmCAAGUUAAAAUAAGGCUAGUCCGUUAUCA | 89 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000483 | mU*mC*mC*ACUCAUUCUUGGCAGGAGUUUUAGAmGmCmUmAmGmAmAmAmUmAmGmCAAGUUAAAAUAAGGCUAGUCCGUUAUCA | 90 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000484 | mA*mG*mA*CACCAAAUCUUACUGGAGUUUUAGAmGmCmUmAmGmAmAmAmUmAmGmCAAGUUAAAAUAAGGCUAGUCCGUUAUCA | 91 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000485 | mC*mC*mU*CCUCUGCCUUGCUGGACGUUUUAGAmGmCmUmAmGmAmAmAmUmAmGmCAAGUUAAAAUAAGGCUAGUCCGUUAUCA | 92 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000486 | mA*mC*mA*CAAAUACCAGUCCAGCAGUUUUAGAmGmCmUmAmGmAmAmAmUmAmGmCAAGUUAAAAUAAGGCUAGUCCGUUAUCA | 93 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000487 | mU*mU*mC*UUUGGCAACUUACCCAGGUUUUAGAmGmCmUmAmGmAmAmAmUmAmGmCAAGUUAAAAUAAGGCUAGUCCGUUAUCA | 94 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000488 | mA*mA*mA*GUUCUAGAUGCUGUCCGGUUUUAGAmGmCmUmAmGmAmAmAmUmAmGmCAAGUUAAAAUAAGGCUAGUCCGUUAUCA | 95 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000489 | mU*mU*mU*GACCAUCAGAGGACACUGUUUUAGAmGmCmUmAmGmAmAmAmUmAmGmCAAGUUAAAAUAAGGCUAGUCCGUUAUCA | 96 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000490 | mA*mA*mA*UAGACACCAAAUCUUACGUUUUAGAmGmCmUmAmGmAmAmAmUmAmGmCAAGUUAAAAUAAGGCUAGUCCGUUAUCA | 97 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000491 | mA*mU*mA*CCAGUCCAGCAAGGCAGGUUUUAGAmGmCmUmAmGmAmAmAmUmAmGmCAAGUUAAAAUAAGGCUAGUCCGUUAUCA | 98 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000492 | mC*mU*mU*CUCUACACCCAGGGCACGUUUUAGAmGmCmUmAmGmAmAmAmUmAmGmCAAGUUAAAAUAAGGCUAGUCCGUUAUCA | 99 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000493 | mA*mA*mG*UGCCUUCCAGUAAGAUUGUUUUAGAmGmCmUmAmGmAmAmAmUmAmGmCAAGUUAAAAUAAGGCUAGUCCGUUAUCA | 100 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000494 | mG*mU*mG*AGUCUGGAGAGCUGCAUGUUUUAGAmGmCmUmAmGmAmAmAmUmAmGmCAAGUUAAAAUAAGGCUAGUCCGUUAUCA | 101 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000495 | mC*mA*mG*AGGACACUUGGAUUCACGUUUUAGAmGmCmUmAmGmAmAmAmUmAmGmCAAGUUAAAAUAAGGCUAGUCCGUUAUCA | 102 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000496 | mG*mG*mC*CGUGCAUGUGUUCAGAAGUUUUAGAmGmCmUmAmGmAmAmAmUmAmGmCAAGUUAAAAUAAGGCUAGUCCGUUAUCA | 103 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000497 | mC*mU*mG*CUCCUCCUCUGCCUUGCGUUUUAGAmGmCmUmAmGmAmAmAmUmAmGmCAAGUUAAAAUAAGGCUAGUCCGUUAUCA | 104 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000498 | mA*mG*mU*GAGUCUGGAGAGCUGCAGUUUUAGAmGmCmUmAmGmAmAmAmUmAmGmCAAGUUAAAAUAAGGCUAGUCCGUUAUCA | 105 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000499 | mU*mG*mA*AUCCAAGUGUCCUCUGAGUUUUAGAmGmCmUmAmGmAmAmAmUmAmGmCAAGUUAAAAUAAGGCUAGUCCGUUAUCA | 106 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000500 | mC*mC*mA*GUCCAGCAAGGCAGAGGGUUUUAGAmGmCmUmAmGmAmAmAmUmAmGmCAAGUUAAAAUAAGGCUAGUCCGUUAUCA | 107 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000501 | mU*mC*mA*CAGAAACACUCACCGUAGUUUUAGAmGmCmUmAmGmAmAmAmUmAmGmCAAGUUAAAAUAAGGCUAGUCCGUUAUCA | 108 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000567 | mG*mA*mA*AGGCUGCUGAUGACACCGUUUUAGAmGmCmUmAmGmAmAmAmUmAmGmCAAGUUAAAAUAAGGCUAGUCCGUUAUCA | 109 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000568 | mG*mG*mC*UGUCGUCACCAAUCCCAGUUUUAGAmGmCmUmAmGmAmAmAmUmAmGmCAAGUUAAAAUAAGGCUAGUCCGUUAUCA | 110 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000570 | mC*mA*mU*UGAUGGCAGGACUGCCUGUUUUAGAmGmCmUmAmGmAmAmAmUmAmGmCAAGUUAAAAUAAGGCUAGUCCGUUAUCA | 111 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000571 | mG*mU*mC*ACAGAAACACUCACCGUGUUUUAGAmGmCmUmAmGmAmAmAmUmAmGmCAAGUUAAAAUAAGGCUAGUCCGUUAUCA | 112 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000572 | mC*mC*mC*CUACUCCUAUUCCACCAGUUUUAGAmGmCmUmAmGmAmAmAmUmAmGmCAAGUUAAAAUAAGGCUAGUCCGUUAUCA | 113 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Human TTR | | |
| G000502 | mA*mC*mA*CAAAUACCAGUCCAGCGGUUUUAGAmGmCmUmAmGmAmAmAmUmAmGmCAAGUUAAAAUAAGGCUAGUCCGUUAUCA | 114 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000503 | mA*mA*mA*AGGCUGCUGAUGAGACCGUUUUAGAmGmCmUmAmGmAmAmAmUmAmGmCAAGUUAAAAUAAGGCUAGUCCGUUAUCA | 115 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000504 | mA*mA*mA*GGCUGCUGAUGAGACCUGUUUUAGAmGmCmUmAmGmAmAmAmUmAmGmCAAGUUAAAAUAAGGCUAGUCCGUUAUCA | 116 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000505 | mC*mA*mU*UGACAGCAGGACUGCCUGUUUUAGAmGmCmUmAmGmAmAmAmUmAmGmCAAGUUAAAAUAAGGCUAGUCCGUUAUCA | 117 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000506 | mA*mU*mA*CCAGUCCAGCGAGGCAGGUUUUAGAmGmCmUmAmGmAmAmAmUmAmGmCAAGUUAAAAUAAGGCUAGUCCGUUAUCA | 118 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000507 | mC*mC*mA*GUCCAGCGAGGCAGAGGGUUUUAGAmGmCmUmAmGmAmAmAmUmAmGmCAAGUUAAAAUAAGGCUAGUCCGUUAUCA | 119 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000508 | mC*mC*mU*CCUCUGCCUCGCUGGACGUUUUAGAmGmCmUmAmGmAmAmAmUmAmGmCAAGUUAAAAUAAGGCUAGUCCGUUAUCA | 120 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000509 | mA*mA*mA*GUUCUAGAUGCCGUCCGGUUUUAGAmGmCmUmAmGmAmAmAmUmAmGmCAAGUUAAAAUAAGGCUAGUCCGUUAUCA | 121 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000510 | mA*mC*mU*UGUCUUCUCUAUACCCAGUUUUAGAmGmCmUmAmGmAmAmAmUmAmGmCAAGUUAAAAUAAGGCUAGUCCGUUAUCA | 122 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000511 | mA*mA*mG*UGACUUCCAGUAAGAUUGUUUUAGAmGmCmUmAmGmAmAmAmUmAmGmCAAGUUAAAAUAAGGCUAGUCCGUUAUCA | 123 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Cyno TTR | | |
| G000282 | mU*mU*mA*CAGCCACGUCUACAGCAGUUUUAGAmGmCmUmAmGmAmAmAmUmAmGmCAAGUUAAAAUAAGGCUAGUCCGUUAUCA | 124 |
| sgRNA | mAmCmUmUmGGmUmGmGmCmAmCmCmGmAmGmUmCmGmGmUmGmCmU*mU*mU*mU | |
| modified | | |
| sequence | | |
| targeting | | |
| Mouse TTR | | |
| exemplary | GUUUUAGAGCUAGAAAUAGCAAGUUAAAAUAAGGCUAGUCCGUUAUCAACUUGAAAAAUGGCACCGAGUCGGUGCUUUU | 125 |
| nucleotide | | |
| sequence | | |
| following | | |
| the 3′ end | | |
| of the Guide | | |
| Sequence to | | |
| form a sgRNA | | |
| exemplary | GUUUUAGAGCUAUGCUGUUUUG | 126 |
| nucleotide | | |
| sequence | | |
| following | | |
| the 3′ end | | |
| of the Guide | | |
| Sequence to | | |
| form a crRNA | | |
| | Not used | 127 to 200 |
| Cas9 DNA | ATGGACAAGAAGTACAGCATCGGACTGGACATCGGAACAAACAGCGTCGGATGGGCAGTCATCACAGACGAATACAAGGTCCCGAG | 201 |
| coding | CAAGAAGTTCAAGGTCCTGGGAAACACAGACAGACACAGCATCAAGAAGAACCTGATCGGAGCACTGCTGTTCGACAGCGGAGAAA | |
| sequence 2 | CAGCAGAAGCAACAAGACTGAAGAGAACAGCAAGAAGAAGATACACAAGAAGAAAGAACAGAATCTGCTACCTGCAGGAAATCTTC | |
| | AGCAACGAAATGGCAAAGGTCGACGACAGCTTCTTCCACAGACTGGAAGAAAGCTTCCTGGTCGAAGAAGACAAGAAGCACGAAAG | |
| | ACACCCGATCTTCGGAAACATCGTCGACGAAGTCGCATACCACGAAAAGTACCCGACAATCTACCACCTGAGAAAGAAGCTGGTCG | |
| | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCG+AGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCCGAAGAAGAAG | |
| | AGAAAGGTCTAG | |
| Cas9 DNA coding | ATGGATAAGAAGTACTCAATCGGGCTGGATATCGGAACTAATTCCGTGGGTTGGGCAGTGATCACGGATGAATACAAAGTGCCGTC | 202 |
| sequence 1 | CAAGAAGTTCAAGGTCCTGGGGAACACCGATAGACACAGCATCAAGAAAAATCTCATCGGAGCCCTGCTGTTTGACTCCGGCGAAA | |
| | CCGCAGAAGCGACCCGGCTCAAACGTACCGCGAGGCGACGCTACACCCGGCGGAAGAATCGCATCTGCTATCTGCAAGAGATCTTT | |
| | TCGAACGAAATGGCAAAGGTCGACGACAGCTTCTTCCACCGCCTGGAAGAATCTTTCCTGGTGGAGGAGGACAAGAAGCATGAACG | |
| | GCATCCTATCTTTGGAAACATCGTCGACGAAGTGGCGTACCACGAAAAGTACCCGACCATCTACCATCTGCGGAAGAAGTTGGTTG | |
| | ACTCAACTGACAAGGCCGACCTCAGATTGATCTACTTGGCCCTCGCCCATATGATCAAATTCCGCGGACACTTCCTGATCGAAGGC | |
| | GATCTGAACCCTGATAACTCCGACGTGGATAAGCTTTTCATTCAACTGGTGCAGACCTACAACCAACTGTTCGAAGAAAACCCAAT | |
| | CAATGCTAGCGGCGTCGATGCCAAGGCCATCCTGTCCGCCCGGCTGTCGAAGTCGCGGCGCCTCGAAAACCTGATCGCACAGCTGC | |
| | CGGGAGAGAAAAAGAACGGACTTTTCGGCAACTTGATCGCTCTCTCACTGGGACTCACTCCCAATTTCAAGTCCAATTTTGACCTG | |
| | GCCGAGGACGCGAAGCTGCAACTCTCAAAGGACACCTACGACGACGACTTGGACAATTTGCTGGCACAAATTGGCGATCAGTACGC | |
| | GGATCTGTTCCTTGCCGCTAAGAACCTTTCGGACGCAATCTTGCTGTCCGATATCCTGCGCGTGAACACCGAAATAACCAAAGCGC | |
| | CGCTTAGCGCCTCGATGATTAAGCGGTACGACGAGCATCACCAGGATCTCACGCTGCTCAAAGCGCTCGTGAGACAGCAACTGCCT | |
| | GAAAAGTACAAGGAGATCTTCTTCGACCAGTCCAAGAATGGGTACGCAGGGTACATCGATGGAGGCGCTAGCCAGGAAGAGTTCTA | |
| | TAAGTTCATCAAGCCAATCCTGGAAAAGATGGACGGAACCGAAGAACTGCTGGTCAAGCTGAACAGGGAGGATCTGCTCCGGAAAC | |
| | AGAGAACCTTTGACAACGGATCCATTCCCCACCAGATCCATCTGGGTGAGCTGCACGCCATCTTGCGGCGCCAGGAGGACTTTTAC | |
| | CCATTCCTCAAGGACAACCGGGAAAAGATCGAGAAAATTCTGACGTTCCGCATCCCGTATTACGTGGGCCCACTGGCGCGCGGCAA | |
| | TTCGCGCTTCGCGTGGATGACTAGAAAATCAGAGGAAACCATCACTCCTTGGAATTTCGAGGAAGTTGTGGATAAGGGAGCTTCGG | |
| | CACAAAGCTTCATCGAACGAATGACCAACTTCGACAAGAATCTCCCAAACGAGAAGGTGCTTCCTAAGCACAGCCTCCTTTACGAA | |
| | TACTTCACTGTCTACAACGAACTGACTAAAGTGAAATACGTTACTGAAGGAATGAGGAAGCCGGCCTTTCTGTCCGGAGAACAGAA | |
| | GAAAGCAATTGTCGATCTGCTGTTCAAGACCAACCGCAAGGTGACCGTCAAGCAGCTTAAAGAGGACTACTTCAAGAAGATCGAGT | |
| | GTTTCGACTCAGTGGAAATCAGCGGGGTGGAGGACAGATTCAACGCTTCGCTGGGAACCTATCATGATCTCCTGAAGATCATCAAG | |
| | GACAAGGACTTCCTTGACAACGAGGAGAACGAGGACATCCTGGAAGATATCGTCCTGACCTTGACCCTTTTCGAGGATCGCGAGAT | |
| | GATCGAGGAGAGGCTTAAGACCTACGCTCATCTCTTCGACGATAAGGTCATGAAACAACTCAAGCGCCGCCGGTACACTGGTTGGG | |
| | GCCGCCTCTCCCGCAAGCTGATCAACGGTATTCGCGATAAACAGAGCGGTAAAACTATCCTGGATTTCCTCAAATCGGATGGCTTC | |
| | GCTAATCGTAACTTCATGCAATTGATCCACGACGACAGCCTGACCTTTAAGGAGGACATCCAAAAAGCACAAGTGTCCGGACAGGG | |
| | AGACTCACTCCATGAACACATCGCGAATCTGGCCGGTTCGCCGGCGATTAAGAAGGGAATTCTGCAAACTGTGAAGGTGGTCGACG | |
| | AGCTGGTGAAGGTCATGGGACGGCACAAACCGGAGAATATCGTGATTGAAATGGCCCGAGAAAACCAGACTACCCAGAAGGGCCAG | |
| | AAAAACTCCCGCGAAAGGATGAAGCGGATCGAAGAAGGAATCAAGGAGCTGGGCAGCCAGATCCTGAAAGAGCACCCGGTGGAAAA | |
| | CACGCAGCTGCAGAACGAGAAGCTCTACCTGTACTATTTGCAAAATGGACGGGACATGTACGTGGACCAAGAGCTGGACATCAATC | |
| | GGTTGTCTGATTACGACGTGGACCACATCGTTCCACAGTCCTTTCTGAAGGATGACTCGATCGATAACAAGGTGTTGACTCGCAGC | |
| | GACAAGAACAGAGGGAAGTCAGATAATGTGCCATCGGAGGAGGTCGTGAAGAAGATGAAGAATTACTGGCGGCAGCTCCTGAATGC | |
| | GAAGCTGATTACCCAGAGAAAGTTTGACAATCTCACTAAAGCCGAGCGCGGCGGACTCTCAGAGCTGGATAAGGCTGGATTCATCA | |
| | AACGGCAGCTGGTCGAGACTCGGCAGATTACCAAGCACGTGGCGCAGATCTTGGACTCCCGCATGAACACTAAATACGACGAGAAC | |
| | GATAAGCTCATCCGGGAAGTGAAGGTGATTACCCTGAAAAGCAAACTTGTGTCGGACTTTCGGAAGGACTTTCAGTTTTACAAAGT | |
| | GAGAGAAATCAACAACTACCATCACGCGCATGACGCATACCTCAACGCTGTGGTCGGTACCGCCCTGATCAAAAAGTACCCTAAAC | |
| | TTGAATCGGAGTTTGTGTACGGAGACTACAAGGTCTACGACGTGAGGAAGATGATAGCCAAGTCCGAACAGGAAATCGGGAAAGCA | |
| | ACTGCGAAATACTTCTTTTACTCAAACATCATGAACTTTTTCAAGACTGAAATTACGCTGGCCAATGGAGAAATCAGGAAGAGGCC | |
| | ACTGATCGAAACTAACGGAGAAACGGGCGAAATCGTGTGGGACAAGGGCAGGGACTTCGCAACTGTTCGCAAAGTGCTCTCTATGC | |
| | CGCAAGTCAATATTGTGAAGAAAACCGAAGTGCAAACCGGCGGATTTTCAAAGGAATCGATCCTCCCAAAGAGAAATAGCGACAAG | |
| | CTCATTGCACGCAAGAAAGACTGGGACCCGAAGAAGTACGGAGGATTCGATTCGCCGACTGTCGCATACTCCGTCCTCGTGGTGGC | |
| | CAAGGTGGAGAAGGGAAAGAGCAAAAAGCTCAAATCCGTCAAAGAGCTGCTGGGGATTACCATCATGGAACGATCCTCGTTCGAGA | |
| | AGAACCCGATTGATTTCCTCGAGGCGAAGGGTTACAAGGAGGTGAAGAAGGATCTGATCATCAAACTCCCCAAGTACTCACTGTTC | |
| | GAACTGGAAAATGGTCGGAAGCGCATGCTGGCTTCGGCCGGAGAACTCCAAAAAGGAAATGAGCTGGCCTTGCCTAGCAAGTACGT | |
| | CAACTTCCTCTATCTTGCTTCGCACTACGAAAAACTCAAAGGGTCACCGGAAGATAACGAACAGAAGCAGCTTTTCGTGGAGCAGC | |
| | ACAAGCATTATCTGGATGAAATCATCGAACAAATCTCCGAGTTTTCAAAGCGCGTGATCCTCGCCGACGCCAACCTCGACAAAGTC | |
| | CTGTCGGCCTACAATAAGCATAGAGATAAGCCGATCAGAGAACAGGCCGAGAACATTATCCACTTGTTCACCCTGACTAACCTGGG | |
| | AGCCCCAGCCGCCTTCAAGTACTTCGATACTACTATCGATCGCAAAAGATACACGTCCACCAAGGAAGTTCTGGACGCGACCCTGA | |
| | TCCACCAAAGCATCACTGGACTCTACGAAACTAGGATCGATCTGTCGCAGCTGGGTGGCGATGGCGGTGGATCTCCGAAAAAGAAG | |
| | AGAAAGGTGTAATGA | |
| Cas9 amino acid | MDKKYSIGLDIGTNSVGWAVITDEYKVPSKKFKVLGNTDRHSIKKNLIGALLFDSGETAEATRLKRTARRRYTRRKNRICYLQEIF | 203 |
| sequence | SNEMAKVDDSFFHRLEESFLVEEDKKHERHPIFGNIVDEVAYHEKYPTIYHLRKKLVDSTDKADLRLIYLALAHMIKFRGHFLIEG | |
| | DLNPDNSDVDKLFIQLVQTYNQLFEENPINASGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSPKKK | |
| | RKV | |
| Cas9 mRNA | AUGGACAAGAAGUACAGCAUCGGACUGGACAUCGGAACAAACAGCGUCGGAUGGGCAGUCAUCACAGACGAAUACAAGGUCCCGAG | 204 |
| open reading | CAAGAAGUUCAAGGUCCUGGGAAACACAGACAGACACAGCAUCAAGAAGAACCUGAUCGGAGCACUGCUGUUCGACAGCGGAGAAA | |
| frame (ORF) | CAGCAGAAGCAACAAGACUGAAGAGAACAGCAAGAAGAAGAUACACAAGAAGAAAGAACAGAAUCUGCUACCUGCAGGAAAUCUUC | |
| 2 | AGCAACGAAAUGGCAAAGGUCGACGACAGCUUCUUCCACAGACUGGAAGAAAGCUUCCUGGUCGAAGAAGACAAGAAGCACGAAAG | |
| | ACACCCGAUCUUCGGAAACAUCGUCGACGAAGUCGCAUACCACGAAAAGUACCCGACAAUCUACCACCUGAGAAAGAAGCUGGUCG | |
| | ACAGCACAGACAAGGCAGACCUGAGACUGAUCUACCUGGCACUGGCACACAUGAUCAAGUUCAGAGGACACUUCCUGAUCGAAGGA | |
| | GACCUGAACCCGGACAACAGCGACGUCGACAAGCUGUUCAUCCAGCUGGUCCAGACAUACAACCAGCUGUUCGAAGAAAACCCGAU | |
| | CAACGCAAGCGGAGUCGACGCAAAGGCAAUCCUGAGCGCAAGACUGAGCAAGAGCAGAAGACUGGAAAACCUGAUCGCACAGCUGC | |
| | CGGGAGAAAAGAAGAACGGACUGUUCGGAAACCUGAUCGCACUGAGCCUGGGACUGACACCGAACUUCAAGAGCAACUUCGACCUG | |
| | GCAGAAGACGCAAAGCUGCAGCUGAGCAAGGACACAUACGACGACGACCUGGACAACCUGCUGGCACAGAUCGGAGACCAGUACGC | |
| | AGACCUGUUCCUGGCAGCAAAGAACCUGAGCGACGCAAUCCUGCUGAGCGACAUCCUGAGAGUCAACACAGAAAUCACAAAGGCAC | |
| | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACGGAGGAGGAAGCCCGAAGAAGAAG | |
| | AGAAAGGUCUAG | |
| Cas9 mRNA | AUGGAUAAGAAGUACUCAAUCGGGCUGGAUAUCGGAACUAAUUCCGUGGGUUGGGCAGUGAUCACGGAUGAAUACAAAGUGCCGUC | 205 |
| ORF 1 | CAAGAAGUUCAAGGUCCUGGGGAACACCGAUAGACACAGCAUCAAGAAAAAUCUCAUCGGAGCCCUGCUGUUUGACUCCGGCGAAA | |
| | CCGCAGAAGCGACCCGGCUCAAACGUACCGCGAGGCGACGCUACACCCGGCGGAAGAAUCGCAUCUGCUAUCUGCAAGAGAUCUUU | |
| | UCGAACGAAAUGGCAAAGGUCGACGACAGCUUCUUCCACCGCCUGGAAGAAUCUUUCCUGGUGGAGGAGGACAAGAAGCAUGAACG | |
| | GCAUCCUAUCUUUGGAAACAUCGUCGACGAAGUGGCGUACCACGAAAAGUACCCGACCAUCUACCAUCUGCGGAAGAAGUUGGUUG | |
| | ACUCAACUGACAAGGCCGACCUCAGAUUGAUCUACUUGGCCCUCGCCCAUAUGAUCAAAUUCCGCGGACACUUCCUGAUCGAAGGC | |
| | GAUCUGAACCCUGAUAACUCCGACGUGGAUAAGCUUUUCAUUCAACUGGUGCAGACCUACAACCAACUGUUCGAAGAAAACCCAAU | |
| | CAAUGCUAGCGGCGUCGAUGCCAAGGCCAUCCUGUCCGCCCGGCUGUCGAAGUCGCGGCGCCUCGAAAACCUGAUCGCACAGCUGC | |
| | CGGGAGAGAAAAAGAACGGACUUUUCGGCAACUUGAUCGCUCUCUCACUGGGACUCACUCCCAAUUUCAAGUCCAAUUUUGACCUG | |
| | GCCGAGGACGCGAAGCUGCAACUCUCAAAGGACACCUACGACGACGACUUGGACAAUUUGCUGGCACAAAUUGGCGAUCAGUACGC | |
| | GGAUCUGUUCCUUGCCGCUAAGAACCUUUCGGACGCAAUCUUGCUGUCCGAUAUCCUGCGCGUGAACACCGAAAUAACCAAAGCGC | |
| | CGCUUAGCGCCUCGAUGAUUAAGCGGUACGACGAGCAUCACCAGGAUCUCACGCUGCUCAAAGCGCUCGUGAGACAGCAACUGCCU | |
| | GAAAAGUACAAGGAGAUCUUCUUCGACCAGUCCAAGAAUGGGUACGCAGGGUACAUCGAUGGAGGCGCUAGCCAGGAAGAGUUCUA | |
| | UAAGUUCAUCAAGCCAAUCCUGGAAAAGAUGGACGGAACCGAAGAACUGCUGGUCAAGCUGAACAGGGAGGAUCUGCUCCGGAAAC | |
| | AGAGAACCUUUGACAACGGAUCCAUUCCCCACCAGAUCCAUCUGGGUGAGCUGCACGCCAUCUUGCGGCGCCAGGAGGACUUUUAC | |
| | CCAUUCCUCAAGGACAACCGGGAAAAGAUCGAGAAAAUUCUGACGUUCCGCAUCCCGUAUUACGUGGGCCCACUGGCGCGCGGCAA | |
| | UUCGCGCUUCGCGUGGAUGACUAGAAAAUCAGAGGAAACCAUCACUCCUUGGAAUUUCGAGGAAGUUGUGGAUAAGGGAGCUUCGG | |
| | CACAAAGCUUCAUCGAACGAAUGACCAACUUCGACAAGAAUCUCCCAAACGAGAAGGUGCUUCCUAAGCACAGCCUCCUUUACGAA | |
| | UACUUCACUGUCUACAACGAACUGACUAAAGUGAAAUACGUUACUGAAGGAAUGAGGAAGCCGGCCUUUCUGUCCGGAGAACAGAA | |
| | GAAAGCAAUUGUCGAUCUGCUGUUCAAGACCAACCGCAAGGUGACCGUCAAGCAGCUUAAAGAGGACUACUUCAAGAAGAUCGAGU | |
| | GUUUCGACUCAGUGGAAAUCAGCGGGGUGGAGGACAGAUUCAACGCUUCGCUGGGAACCUAUCAUGAUCUCCUGAAGAUCAUCAAG | |
| | GACAAGGACUUCCUUGACAACGAGGAGAACGAGGACAUCCUGGAAGAUAUCGUCCUGACCUUGACCCUUUUCGAGGAUCGCGAGAU | |
| | GAUCGAGGAGAGGCUUAAGACCUACGCUCAUCUCUUCGACGAUAAGGUCAUGAAACAACUCAAGCGCCGCCGGUACACUGGUUGGG | |
| | GCCGCCUCUCCCGCAAGCUGAUCAACGGUAUUCGCGAUAAACAGAGCGGUAAAACUAUCCUGGAUUUCCUCAAAUCGGAUGGCUUC | |
| | GCUAAUCGUAACUUCAUGCAAUUGAUCCACGACGACAGCCUGACCUUUAAGGAGGACAUCCAAAAAGCACAAGUGUCCGGACAGGG | |
| | AGACUCACUCCAUGAACACAUCGCGAAUCUGGCCGGUUCGCCGGCGAUUAAGAAGGGAAUUCUGCAAACUGUGAAGGUGGUCGACG | |
| | AGCUGGUGAAGGUCAUGGGACGGCACAAACCGGAGAAUAUCGUGAUUGAAAUGGCCCGAGAAAACCAGACUACCCAGAAGGGCCAG | |
| | AAAAACUCCCGCGAAAGGAUGAAGCGGAUCGAAGAAGGAAUCAAGGAGCUGGGCAGCCAGAUCCUGAAAGAGCACCCGGUGGAAAA | |
| | CACGCAGCUGCAGAACGAGAAGCUCUACCUGUACUAUUUGCAAAAUGGACGGGACAUGUACGUGGACCAAGAGCUGGACAUCAAUC | |
| | GGUUGUCUGAUUACGACGUGGACCACAUCGUUCCACAGUCCUUUCUGAAGGAUGACUCGAUCGAUAACAAGGUGUUGACUCGCAGC | |
| | GACAAGAACAGAGGGAAGUCAGAUAAUGUGCCAUCGGAGGAGGUCGUGAAGAAGAUGAAGAAUUACUGGCGGCAGCUCCUGAAUGC | |
| | GAAGCUGAUUACCCAGAGAAAGUUUGACAAUCUCACUAAAGCCGAGCGCGGCGGACUCUCAGAGCUGGAUAAGGCUGGAUUCAUCA | |
| | AACGGCAGCUGGUCGAGACUCGGCAGAUUACCAAGCACGUGGCGCAGAUCUUGGACUCCCGCAUGAACACUAAAUACGACGAGAAC | |
| | GAUAAGCUCAUCCGGGAAGUGAAGGUGAUUACCCUGAAAAGCAAACUUGUGUCGGACUUUCGGAAGGACUUUCAGUUUUACAAAGU | |
| | GAGAGAAAUCAACAACUACCAUCACGCGCAUGACGCAUACCUCAACGCUGUGGUCGGUACCGCCCUGAUCAAAAAGUACCCUAAAC | |
| | UUGAAUCGGAGUUUGUGUACGGAGACUACAAGGUCUACGACGUGAGGAAGAUGAUAGCCAAGUCCGAACAGGAAAUCGGGAAAGCA | |
| | ACUGCGAAAUACUUCUUUUACUCAAACAUCAUGAACUUUUUCAAGACUGAAAUUACGCUGGCCAAUGGAGAAAUCAGGAAGAGGCC | |
| | ACUGAUCGAAACUAACGGAGAAACGGGCGAAAUCGUGUGGGACAAGGGCAGGGACUUCGCAACUGUUCGCAAAGUGCUCUCUAUGC | |
| | CGCAAGUCAAUAUUGUGAAGAAAACCGAAGUGCAAACCGGCGGAUUUUCAAAGGAAUCGAUCCUCCCAAAGAGAAAUAGCGACAAG | |
| | CUCAUUGCACGCAAGAAAGACUGGGACCCGAAGAAGUACGGAGGAUUCGAUUCGCCGACUGUCGCAUACUCCGUCCUCGUGGUGGC | |
| | CAAGGUGGAGAAGGGAAAGAGCAAAAAGCUCAAAUCCGUCAAAGAGCUGCUGGGGAUUACCAUCAUGGAACGAUCCUCGUUCGAGA | |
| | AGAACCCGAUUGAUUUCCUCGAGGCGAAGGGUUACAAGGAGGUGAAGAAGGAUCUGAUCAUCAAACUCCCCAAGUACUCACUGUUC | |
| | GAACUGGAAAAUGGUCGGAAGCGCAUGCUGGCUUCGGCCGGAGAACUCCAAAAAGGAAAUGAGCUGGCCUUGCCUAGCAAGUACGU | |
| | CAACUUCCUCUAUCUUGCUUCGCACUACGAAAAACUCAAAGGGUCACCGGAAGAUAACGAACAGAAGCAGCUUUUCGUGGAGCAGC | |
| | ACAAGCAUUAUCUGGAUGAAAUCAUCGAACAAAUCUCCGAGUUUUCAAAGCGCGUGAUCCUCGCCGACGCCAACCUCGACAAAGUC | |
| | CUGUCGGCCUACAAUAAGCAUAGAGAUAAGCCGAUCAGAGAACAGGCCGAGAACAUUAUCCACUUGUUCACCCUGACUAACCUGGG | |
| | AGCCCCAGCCGCCUUCAAGUACUUCGAUACUACUAUCGAUCGCAAAAGAUACACGUCCACCAAGGAAGUUCUGGACGCGACCCUGA | |
| | UCCACCAAAGCAUCACUGGACUCUACGAAACUAGGAUCGAUCUGUCGCAGCUGGGUGGCGAUGGCGGUGGAUCUCCGAAAAAGAAG | |
| | AGAAAGGUGUAAUGA | |
| Cas9 nickase | MDKKYSIGLAIGTNSVGWAVITDEYKVPSKKFKVLGNTDRHSIKKNLIGALLFDSGETAEATRLKRTARRRYTRRKNRICYLQEIF | 206 |
| (D10A) amino | SNEMAKVDDSFFHRLEESFLVEEDKKHERHPIFGNIVDEVAYHEKYPTIYHLRKKLVDSTDKADLRLIYLALAHMIKFRGHFLIEG | |
| acid sequence | DLNPDNSDVDKLFIQLVQTYNQLFEENPINASGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSPKKK | |
| | RKV | |
| Cas9 nickase | AUGGACAAGAAGUACAGCAUCGGACUGGCAAUCGGAACAAACAGCGUCGGAUGGGCAGUCAUCACAGACGAAUACAAGGUCCCGAG | 207 |
| (D10A) mRNA ORF | CAAGAAGUUCAAGGUCCUGGGAAACACAGACAGACACAGCAUCAAGAAGAACCUGAUCGGAGCACUGCUGUUCGACAGCGGAGAAA | |
| | CAGCAGAAGCAACAAGACUGAAGAGAACAGCAAGAAGAAGAUACACAAGAAGAAAGAACAGAAUCUGCUACCUGCAGGAAAUCUUC | |
| | AGCAACGAAAUGGCAAAGGUCGACGACAGCUUCUUCCACAGACUGGAAGAAAGCUUCCUGGUCGAAGAAGACAAGAAGCACGAAAG | |
| | ACACCCGAUCUUCGGAAACAUCGUCGACGAAGUCGCAUACCACGAAAAGUACCCGACAAUCUACCACCUGAGAAAGAAGCUGGUCG | |
| | ACAGCACAGACAAGGCAGACCUGAGACUGAUCUACCUGGCACUGGCACACAUGAUCAAGUUCAGAGGACACUUCCUGAUCGAAGGA | |
| | GACCUGAACCCGGACAACAGCGACGUCGACAAGCUGUUCAUCCAGCUGGUCCAGACAUACAACCAGCUGUUCGAAGAAAACCCGAU | |
| | CAACGCAAGCGGAGUCGACGCAAAGGCAAUCCUGAGCGCAAGACUGAGCAAGAGCAGAAGACUGGAAAACCUGAUCGCACAGCUGC | |
| | CGGGAGAAAAGAAGAACGGACUGUUCGGAAACCUGAUCGCACUGAGCCUGGGACUGACACCGAACUUCAAGAGCAACUUCGACCUG | |
| | GCAGAAGACGCAAAGCUGCAGCUGAGCAAGGACACAUACGACGACGACCUGGACAACCUGCUGGCACAGAUCGGAGACCAGUACGC | |
| | AGACCUGUUCCUGGCAGCAAAGAACCUGAGCGACGCAAUCCUGCUGAGCGACAUCCUGAGAGUCAACACAGAAAUCACAAAGGCAC | |
| | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAAaAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACGGAGGAGGAAGCCCGAAGAAGAAG | |
| | AGAAAGGUCUAG | |
| dCas9 (D10A | MDKKYSIGLAIGTNSVGWAVITDEYKVPSKKFKVLGNTDRHSIKKNLIGALLFDSGETAEATRLKRTARRRYTRRKNRICYLQEIF | 208 |
| H840A) amino acid | SNEMAKVDDSFFHRLEESFLVEEDKKHERHPIFGNIVDEVAYHEKYPTIYHLRKKLVDSTDKADLRLIYLALAHMIKFRGHFLIEG | |
| sequence | DLNPDNSDVDKLFIQLVQTYNQLFEENPINASGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDAIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSPKKK | |
| | RKV | |
| dCas9 (D10A | AUGGACAAGAAGUACAGCAUCGGACUGGCAAUCGGAACAAACAGCGUCGGAUGGGCAGUCAUCACAGACGAAUACAAGGUCCCGAG | 209 |
| H840A) mRNA | CAAGAAGUUCAAGGUCCUGGGAAACACAGACAGACACAGCAUCAAGAAGAACCUGAUCGGAGCACUGCUGUUCGACAGCGGAGAAA | |
| ORF | CAGCAGAAGCAACAAGACUGAAGAGAACAGCAAGAAGAAGAUACACAAGAAGAAAGAACAGAAUCUGCUACCUGCAGGAAAUCUUC | |
| | AGCAACGAAAUGGCAAAGGUCGACGACAGCUUCUUCCACAGACUGGAAGAAAGCUUCCUGGUCGAAGAAGACAAGAAGCACGAAAG | |
| | ACACCCGAUCUUCGGAAACAUCGUCGACGAAGUCGCAUACCACGAAAAGUACCCGACAAUCUACCACCUGAGAAAGAAGCUGGUCG | |
| | ACAGCACAGACAAGGCAGACCUGAGACUGAUCUACCUGGCACUGGCACACAUGAUCAAGUUCAGAGGACACUUCCUGAUCGAAGGA | |
| | GACCUGAACCCGGACAACAGCGACGUCGACAAGCUGUUCAUCCAGCUGGUCCAGACAUACAACCAGCUGUUCGAAGAAAACCCGAU | |
| | CAACGCAAGCGGAGUCGACGCAAAGGCAAUCCUGAGCGCAAGACUGAGCAAGAGCAGAAGACUGGAAAACCUGAUCGCACAGCUGC | |
| | CGGGAGAAAAGAAGAACGGACUGUUCGGAAACCUGAUCGCACUGAGCCUGGGACUGACACCGAACUUCAAGAGCAACUUCGACCUG | |
| | GCAGAAGACGCAAAGCUGCAGCUGAGCAAGGACACAUACGACGACGACCUGGACAACCUGCUGGCACAGAUCGGAGACCAGUACGC | |
| | AGACCUGUUCCUGGCAGCAAAGAACCUGAGCGACGCAAUCCUGCUGAGCGACAUCCUGAGAGUCAACACAGAAAUCACAAAGGCAC | |
| | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACGCAAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACGGAGGAGGAAGCCCGAAGAAGAAG | |
| | AGAAAGGUCUAG | |
| Cas9 bare | GACAAGAAGUACAGCAUCGGACUGGACAUCGGAACAAACAGCGUCGGAUGGGCAGUCAUCACAGACGAAUACAAGGUCCCGAGCAA | 210 |
| coding | GAAGUUCAAGGUCCUGGGAAACACAGACAGACACAGCAUCAAGAAGAACCUGAUCGGAGCACUGCUGUUCGACAGCGGAGAAACAG | |
| sequence | CAGAAGCAACAAGACUGAAGAGAACAGCAAGAAGAAGAUACACAAGAAGAAAGAACAGAAUCUGCUACCUGCAGGAAAUCUUCAGC | |
| | AACGAAAUGGCAAAGGUCGACGACAGCUUCUUCCACAGACUGGAAGAAAGCUUCCUGGUCGAAGAAGACAAGAAGCACGAAAGACA | |
| | CCCGAUCUUCGGAAACAUCGUCGACGAAGUCGCAUACCACGAAAAGUACCCGACAAUCUACCACCUGAGAAAGAAGCUGGUCGACA | |
| | GCACAGACAAGGCAGACCUGAGACUGAUCUACCUGGCACUGGCACACAUGAUCAAGUUCAGAGGACACUUCCUGAUCGAAGGAGAC | |
| | CUGAACCCGGACAACAGCGACGUCGACAAGCUGUUCAUCCAGCUGGUCCAGACAUACAACCAGCUGUUCGAAGAAAACCCGAUCAA | |
| | CGCAAGCGGAGUCGACGCAAAGGCAAUCCUGAGCGCAAGACUGAGCAAGAGCAGAAGACUGGAAAACCUGAUCGCACAGCUGCCGG | |
| | GAGAAAAGAAGAACGGACUGUUCGGAAACCUGAUCGCACUGAGCCUGGGACUGACACCGAACUUCAAGAGCAACUUCGACCUGGCA | |
| | GAAGACGCAAAGCUGCAGCUGAGCAAGGACACAUACGACGACGACCUGGACAACCUGCUGGCACAGAUCGGAGACCAGUACGCAGA | |
| | CCUGUUCCUGGCAGCAAAGAACCUGAGCGACGCAAUCCUGCUGAGCGACAUCCUGAGAGUCAACACAGAAAUCACAAAGGCACCGC | |
| | UGAGCGCAAGCAUGAUCAAGAGAUACGACGAACACCACCAGGACCUGACACUGCUGAAGGCACUGGUCAGACAGCAGCUGCCGGAA | |
| | AAGUACAAGGAAAUCUUCUUCGACCAGAGCAAGAACGGAUACGCAGGAUACAUCGACGGAGGAGCAAGCCAGGAAGAAUUCUACAA | |
| | GUUCAUCAAGCCGAUCCUGGAAAAGAUGGACGGAACAGAAGAACUGCUGGUCAAGCUGAACAGAGAAGACCUGCUGAGAAAGCAGA | |
| | GAACAUUCGACAACGGAAGCAUCCCGCACCAGAUCCACCUGGGAGAACUGCACGCAAUCCUGAGAAGACAGGAAGACUUCUACCCG | |
| | UUCCUGAAGGACAACAGAGAAAAGAUCGAAAAGAUCCUGACAUUCAGAAUCCCGUACUACGUCGGACCGCUGGCAAGAGGAAACAG | |
| | CAGAUUCGCAUGGAUGACAAGAAAGAGCGAAGAAACAAUCACACCGUGGAACUUCGAAGAAGUCGUCGACAAGGGAGCAAGCGCAC | |
| | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGACGGAGGAGGAAGCCCGAAGAAGAAGAGA | |
| | AAGGUC | |
| Cas9 nickase | GACAAGAAGUACAGCAUCGGACUGGCAAUCGGAACAAACAGCGUCGGAUGGGCAGUCAUCACAGACGAAUACAAGGUCCCGAGCAA | 211 |
| bare coding | GAAGUUCAAGGUCCUGGGAAACACAGACAGACACAGCAUCAAGAAGAACCUGAUCGGAGCACUGCUGUUCGACAGCGGAGAAACAG | |
| sequence | CAGAAGCAACAAGACUGAAGAGAACAGCAAGAAGAAGAUACACAAGAAGAAAGAACAGAAUCUGCUACCUGCAGGAAAUCUUCAGC | |
| | AACGAAAUGGCAAAGGUCGACGACAGCUUCUUCCACAGACUGGAAGAAAGCUUCCUGGUCGAAGAAGACAAGAAGCACGAAAGACA | |
| | CCCGAUCUUCGGAAACAUCGUCGACGAAGUCGCAUACCACGAAAAGUACCCGACAAUCUACCACCUGAGAAAGAAGCUGGUCGACA | |
| | GCACAGACAAGGCAGACCUGAGACUGAUCUACCUGGCACUGGCACACAUGAUCAAGUUCAGAGGACACUUCCUGAUCGAAGGAGAC | |
| | CUGAACCCGGACAACAGCGACGUCGACAAGCUGUUCAUCCAGCUGGUCCAGACAUACAACCAGCUGUUCGAAGAAAACCCGAUCAA | |
| | CGCAAGCGGAGUCGACGCAAAGGCAAUCCUGAGCGCAAGACUGAGCAAGAGCAGAAGACUGGAAAACCUGAUCGCACAGCUGCCGG | |
| | GAGAAAAGAAGAACGGACUGUUCGGAAACCUGAUCGCACUGAGCCUGGGACUGACACCGAACUUCAAGAGCAACUUCGACCUGGCA | |
| | GAAGACGCAAAGCUGCAGCUGAGCAAGGACACAUACGACGACGACCUGGACAACCUGCUGGCACAGAUCGGAGACCAGUACGCAGA | |
| | CCUGUUCCUGGCAGCAAAGAACCUGAGCGACGCAAUCCUGCUGAGCGACAUCCUGAGAGUCAACACAGAAAUCACAAAGGCACCGC | |
| | UGAGCGCAAGCAUGAUCAAGAGAUACGACGAACACCACCAGGACCUGACACUGCUGAAGGCACUGGUCAGACAGCAGCUGCCGGAA | |
| | AAGUACAAGGAAAUCUUCUUCGACCAGAGCAAGAACGGAUACGCAGGAUACAUCGACGGAGGAGCAAGCCAGGAAGAAUUCUACAA | |
| | GUUCAUCAAGCCGAUCCUGGAAAAGAUGGACGGAACAGAAGAACUGCUGGUCAAGCUGAACAGAGAAGACCUGCUGAGAAAGCAGA | |
| | GAACAUUCGACAACGGAAGCAUCCCGCACCAGAUCCACCUGGGAGAACUGCACGCAAUCCUGAGAAGACAGGAAGACUUCUACCCG | |
| | UUCCUGAAGGACAACAGAGAAAAGAUCGAAAAGAUCCUGACAUUCAGAAUCCCGUACUACGUCGGACCGCUGGCAAGAGGAAACAG | |
| | CAGAUUCGCAUGGAUGACAAGAAAGAGCGAAGAAACAAUCACACCGUGGAACUUCGAAGAAGUCGUCGACAAGGGAGCAAGCGCAC | |
| | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGACGGAGGAGGAAGCCCGAAGAAGAAGAGA | |
| | AAGGUC | |
| dCas9 bare | GACAAGAAGUACAGCAUCGGACUGGCAAUCGGAACAAACAGCGUCGGAUGGGCAGUCAUCACAGACGAAUACAAGGUCCCGAGCAA | 212 |
| coding | GAAGUUCAAGGUCCUGGGAAACACAGACAGACACAGCAUCAAGAAGAACCUGAUCGGAGCACUGCUGUUCGACAGCGGAGAAACAG | |
| sequence | CAGAAGCAACAAGACUGAAGAGAACAGCAAGAAGAAGAUACACAAGAAGAAAGAACAGAAUCUGCUACCUGCAGGAAAUCUUCAGC | |
| | AACGAAAUGGCAAAGGUCGACGACAGCUUCUUCCACAGACUGGAAGAAAGCUUCCUGGUCGAAGAAGACAAGAAGCACGAAAGACA | |
| | CCCGAUCUUCGGAAACAUCGUCGACGAAGUCGCAUACCACGAAAAGUACCCGACAAUCUACCACCUGAGAAAGAAGCUGGUCGACA | |
| | GCACAGACAAGGCAGACCUGAGACUGAUCUACCUGGCACUGGCACACAUGAUCAAGUUCAGAGGACACUUCCUGAUCGAAGGAGAC | |
| | CUGAACCCGGACAACAGCGACGUCGACAAGCUGUUCAUCCAGCUGGUCCAGACAUACAACCAGCUGUUCGAAGAAAACCCGAUCAA | |
| | CGCAAGCGGAGUCGACGCAAAGGCAAUCCUGAGCGCAAGACUGAGCAAGAGCAGAAGACUGGAAAACCUGAUCGCACAGCUGCCGG | |
| | GAGAAAAGAAGAACGGACUGUUCGGAAACCUGAUCGCACUGAGCCUGGGACUGACACCGAACUUCAAGAGCAACUUCGACCUGGCA | |
| | GAAGACGCAAAGCUGCAGCUGAGCAAGGACACAUACGACGACGACCUGGACAACCUGCUGGCACAGAUCGGAGACCAGUACGCAGA | |
| | CCUGUUCCUGGCAGCAAAGAACCUGAGCGACGCAAUCCUGCUGAGCGACAUCCUGAGAGUCAACACAGAAAUCACAAAGGCACCGC | |
| | UGAGCGCAAGCAUGAUCAAGAGAUACGACGAACACCACCAGGACCUGACACUGCUGAAGGCACUGGUCAGACAGCAGCUGCCGGAA | |
| | AAGUACAAGGAAAUCUUCUUCGACCAGAGCAAGAACGGAUACGCAGGAUACAUCGACGGAGGAGCAAGCCAGGAAGAAUUCUACAA | |
| | GUUCAUCAAGCCGAUCCUGGAAAAGAUGGACGGAACAGAAGAACUGCUGGUCAAGCUGAACAGAGAAGACCUGCUGAGAAAGCAGA | |
| | GAACAUUCGACAACGGAAGCAUCCCGCACCAGAUCCACCUGGGAGAACUGCACGCAAUCCUGAGAAGACAGGAAGACUUCUACCCG | |
| | UUCCUGAAGGACAACAGAGAAAAGAUCGAAAAGAUCCUGACAUUCAGAAUCCCGUACUACGUCGGACCGCUGGCAAGAGGAAACAG | |
| | CAGAUUCGCAUGGAUGACAAGAAAGAGCGAAGAAACAAUCACACCGUGGAACUUCGAAGAAGUCGUCGACAAGGGAGCAAGCGCAC | |
| | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACGCAAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGACGGAGGAGGAAGCCCGAAGAAGAAGAGA | |
| | AAGGUC | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 213 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 | DLNPDNSDVDKLFIQLVQTYNQLFEENPINASGVDAKAILSARLSKSRRLENLIAQLPGEKKNGLFGNLIALSLGLTPNFKSNFDL | |
| (without NLS) | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGD | |
| Cas9 mRNA | AUGGACAAGAAGUACAGCAUCGGACUGGACAUCGGAACAAACAGCGUCGGAUGGGCAGUCAUCACAGACGAAUACAAGGUCCCGAG | 214 |
| ORF encoding | CAAGAAGUUCAAGGUCCUGGGAAACACAGACAGACACAGCAUCAAGAAGAACCUGAUCGGAGCACUGCUGUUCGACAGCGGAGAAA | |
| SEQ ID NO: | CAGCAGAAGCAACAAGACUGAAGAGAACAGCAAGAAGAAGAUACACAAGAAGAAAGAACAGAAUCUGCUACCUGCAGGAAAUCUUC | |
| 13 using | AGCAACGAAAUGGCAAAGGUCGACGACAGCUUCUUCCACAGACUGGAAGAAAGCUUCCUGGUCGAAGAAGACAAGAAGCACGAAAG | |
| minimal | ACACCCGAUCUUCGGAAACAUCGUCGACGAAGUCGCAUACCACGAAAAGUACCCGACAAUCUACCACCUGAGAAAGAAGCUGGUCG | |
| uridine | ACAGCACAGACAAGGCAGACCUGAGACUGAUCUACCUGGCACUGGCACACAUGAUCAAGUUCAGAGGACACUUCCUGAUCGAAGGA | |
| codons as | GACCUGAACCCGGACAACAGCGACGUCGACAAGCUGUUCAUCCAGCUGGUCCAGACAUACAACCAGCUGUUCGAAGAAAACCCGAU | |
| listed in | CAACGCAAGCGGAGUCGACGCAAAGGCAAUCCUGAGCGCAAGACUGAGCAAGAGCAGAAGACUGGAAAACCUGAUCGCACAGCUGC | |
| Table 3, | CGGGAGAAAAGAAGAACGGACUGUUCGGAAACCUGAUCGCACUGAGCCUGGGACUGACACCGAACUUCAAGAGCAACUUCGACCUG | |
| with start | GCAGAAGACGCAAAGCUGCAGCUGAGCAAGGACACAUACGACGACGACCUGGACAACCUGCUGGCACAGAUCGGAGACCAGUACGC | |
| and stop | AGACCUGUUCCUGGCAGCAAAGAACCUGAGCGACGCAAUCCUGCUGAGCGACAUCCUGAGAGUCAACACAGAAAUCACAAAGGCAC | |
| codons | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACUAG | |
| Cas9 coding | GACAAGAAGUACAGCAUCGGACUGGACAUCGGAACAAACAGCGUCGGAUGGGCAGUCAUCACAGACGAAUACAAGGUCCCGAGCAA | 215 |
| sequence | GAAGUUCAAGGUCCUGGGAAACACAGACAGACACAGCAUCAAGAAGAACCUGAUCGGAGCACUGCUGUUCGACAGCGGAGAAACAG | |
| encoding SEQ | CAGAAGCAACAAGACUGAAGAGAACAGCAAGAAGAAGAUACACAAGAAGAAAGAACAGAAUCUGCUACCUGCAGGAAAUCUUCAGC | |
| ID NO: 13 | AACGAAAUGGCAAAGGUCGACGACAGCUUCUUCCACAGACUGGAAGAAAGCUUCCUGGUCGAAGAAGACAAGAAGCACGAAAGACA | |
| using minimal | CCCGAUCUUCGGAAACAUCGUCGACGAAGUCGCAUACCACGAAAAGUACCCGACAAUCUACCACCUGAGAAAGAAGCUGGUCGACA | |
| uridine codons as | GCACAGACAAGGCAGACCUGAGACUGAUCUACCUGGCACUGGCACACAUGAUCAAGUUCAGAGGACACUUCCUGAUCGAAGGAGAC | |
| listed in | CUGAACCCGGACAACAGCGACGUCGACAAGCUGUUCAUCCAGCUGGUCCAGACAUACAACCAGCUGUUCGAAGAAAACCCGAUCAA | |
| Table 3 (no start | CGCAAGCGGAGUCGACGCAAAGGCAAUCCUGAGCGCAAGACUGAGCAAGAGCAGAAGACUGGAAAACCUGAUCGCACAGCUGCCGG | |
| or stop codons; | GAGAAAAGAAGAACGGACUGUUCGGAAACCUGAUCGCACUGAGCCUGGGACUGACACCGAACUUCAAGAGCAACUUCGACCUGGCA | |
| suitable for | GAAGACGCAAAGCUGCAGCUGAGCAAGGACACAUACGACGACGACCUGGACAACCUGCUGGCACAGAUCGGAGACCAGUACGCAGA | |
| inclusion in | CCUGUUCCUGGCAGCAAAGAACCUGAGCGACGCAAUCCUGCUGAGCGACAUCCUGAGAGUCAACACAGAAAUCACAAAGGCACCGC | |
| fusion protein | UGAGCGCAAGCAUGAUCAAGAGAUACGACGAACACCACCAGGACCUGACACUGCUGAAGGCACUGGUCAGACAGCAGCUGCCGGAA | |
| coding sequence) | AAGUACAAGGAAAUCUUCUUCGACCAGAGCAAGAACGGAUACGCAGGAUACAUCGACGGAGGAGCAAGCCAGGAAGAAUUCUACAA | |
| | GUUCAUCAAGCCGAUCCUGGAAAAGAUGGACGGAAGAGAAGAACUGCUGGUCAAGCUGAACAGAGAAGACCUGCUGAGAAAGCAGA | |
| | GAACAUUCGACAACGGAAGCAUCCCGCACCAGAUCCACCUGGGAGAACUGCACGCAAUCCUGAGAAGACAGGAAGACUUCUACCCG | |
| | UUCCUGAAGGACAACAGAGAAAAGAUCGAAAAGAUCCUGACAUUCAGAAUCCCGUACUACGUCGGACCGCUGGCAAGAGGAAACAG | |
| | CAGAUUCGCAUGGAUGACAAGAAAGAGCGAAGAAACAAUCACACCGUGGAACUUCGAAGAAGUCGUCGACAAGGGAGCAAGCGCAC | |
| | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGAC | |
| Amino acid | MDKKYSIGLAIGTNSVGWAVITDEYKVPSKKFKVLGNTDRHSIKKNLIGALLFDSGETAEATRLKRTARRRYTRRKNRICYLQEIF | 216 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 nickase | DLNPDNSDVDKLFIQLVQTYNQLFEENPINASGVDAKAILSARLSKSRRLENLIAQLPGEKKNGLFGNLIALSLGLTPNFKSNFDL | |
| (without | AEDAKLQLSKDTYDDDLDNLLAQIGDQYADLFLAAKNLSDAILLSDILRVNTEITKAPLSASMIKRYDEHHQDLTLLKALVRQQLP | |
| NLS) | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGD | |
| Cas9 nickase | AUGGACAAGAAGUACAGCAUCGGACUGGCAAUCGGAACAAACAGCGUCGGAUGGGCAGUCAUCACAGACGAAUACAAGGUCCCGAG | 217 |
| mRNA ORF | CAAGAAGUUCAAGGUCCUGGGAAACACAGACAGACACAGCAUCAAGAAGAACCUGAUCGGAGCACUGCUGUUCGACAGCGGAGAAA | |
| encoding SEQ | CAGCAGAAGCAACAAGACUGAAGAGAACAGCAAGAAGAAGAUACACAAGAAGAAAGAACAGAAUCUGCUACCUGCAGGAAAUCUUC | |
| ID NO: 16 | AGCAACGAAAUGGCAAAGGUCGACGACAGCUUCUUCCACAGACUGGAAGAAAGCUUCCUGGUCGAAGAAGACAAGAAGCACGAAAG | |
| using | ACACCCGAUCUUCGGAAACAUCGUCGACGAAGUCGCAUACCACGAAAAGUACCCGACAAUCUACCACCUGAGAAAGAAGCUGGUCG | |
| minimal | ACAGCACAGACAAGGCAGACCUGAGACUGAUCUACCUGGCACUGGCACACAUGAUCAAGUUCAGAGGACACUUCCUGAUCGAAGGA | |
| uridine | GACCUGAACCCGGACAACAGCGACGUCGACAAGCUGUUCAUCCAGCUGGUCCAGACAUACAACCAGCUGUUCGAAGAAAACCCGAU | |
| codons as | CAACGCAAGCGGAGUCGACGCAAAGGCAAUCCUGAGCGCAAGACUGAGCAAGAGCAGAAGACUGGAAAACCUGAUCGCACAGCUGC | |
| listed in | CGGGAGAAAAGAAGAACGGACUGUUCGGAAACCUGAUCGCACUGAGCCUGGGACUGACACCGAACUUCAAGAGCAACUUCGACCUG | |
| Table 3, | GCAGAAGACGCAAAGCUGCAGCUGAGCAAGGACACAUACGACGACGACCUGGACAACCUGCUGGCACAGAUCGGAGACCAGUACGC | |
| with start | AGACCUGUUCCUGGCAGCAAAGAACCUGAGCGACGCAAUCCUGCUGAGCGACAUCCUGAGAGUCAACACAGAAAUCACAAAGGCAC | |
| and stop | CGCUGAGCGCAAGCAUGAUCAAGAGAUACGACGAACACCACCAGGACCUGACACUGCUGAAGGCACUGGUCAGACAGCAGCUGCCG | |
| codons | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACUAG | |
| Cas9 nickase | GACAAGAAGUACAGCAUCGGACUGGCAAUCGGAACAAACAGCGUCGGAUGGGCAGUCAUCACAGACGAAUACAAGGUCCCGAGCAA | 218 |
| coding | GAAGUUCAAGGUCCUGGGAAACACAGACAGACACAGCAUCAAGAAGAACCUGAUCGGAGCACUGCUGUUCGACAGCGGAGAAACAG | |
| sequence | CAGAAGCAACAAGACUGAAGAGAACAGCAAGAAGAAGAUACACAAGAAGAAAGAACAGAAUCUGCUACCUGCAGGAAAUCUUCAGC | |
| encoding SEQ | AACGAAAUGGCAAAGGUCGACGACAGCUUCUUCCACAGACUGGAAGAAAGCUUCCUGGUCGAAGAAGACAAGAAGCACGAAAGACA | |
| ID NO: 16 | CCCGAUCUUCGGAAACAUCGUCGACGAAGUCGCAUACCACGAAAAGUACCCGACAAUCUACCACCUGAGAAAGAAGCUGGUCGACA | |
| using | GCACAGACAAGGCAGACCUGAGACUGAUCUACCUGGCACUGGCACACAUGAUCAAGUUCAGAGGACACUUCCUGAUCGAAGGAGAC | |
| minimal | CUGAACCCGGACAACAGCGACGUCGACAAGCUGUUCAUCCAGCUGGUCCAGACAUACAACCAGCUGUUCGAAGAAAACCCGAUCAA | |
| uridine | CGCAAGCGGAGUCGACGCAAAGGCAAUCCUGAGCGCAAGACUGAGCAAGAGCAGAAGACUGGAAAACCUGAUCGCACAGCUGCCGG | |
| codons as | GAGAAAAGAAGAACGGACUGUUCGGAAACCUGAUCGCACUGAGCCUGGGACUGACACCGAACUUCAAGAGCAACUUCGACCUGGCA | |
| listed in | GAAGACGCAAAGCUGCAGCUGAGCAAGGACACAUACGACGACGACCUGGACAACCUGCUGGCACAGAUCGGAGACCAGUACGCAGA | |
| Table 3 (no | CCUGUUCCUGGCAGCAAAGAACCUGAGCGACGCAAUCCUGCUGAGCGACAUCCUGAGAGUCAACACAGAAAUCACAAAGGCACCGC | |
| start or | UGAGCGCAAGCAUGAUCAAGAGAUACGACGAACACCACCAGGACCUGACACUGCUGAAGGCACUGGUCAGACAGCAGCUGCCGGAA | |
| stop codons; | AAGUACAAGGAAAUCUUCUUCGACCAGAGCAAGAACGGAUACGCAGGAUACAUCGACGGAGGAGCAAGCCAGGAAGAAUUCUACAA | |
| suitable for | GUUCAUCAAGCCGAUCCUGGAAAAGAUGGACGGAACAGAAGAACUGCUGGUCAAGCUGAACAGAGAAGACCUGCUGAGAAAGCAGA | |
| inclusion in | GAACAUUCGACAACGGAAGCAUCCCGCACCAGAUCCACCUGGGAGAACUGCACGCAAUCCUGAGAAGACAGGAAGACUUCUACCCG | |
| fusion | UUCCUGAAGGACAACAGAGAAAAGAUCGAAAAGAUCCUGACAUUCAGAAUCCCGUACUACGUCGGACCGCUGGCAAGAGGAAACAG | |
| protein | CAGAUUCGCAUGGAUGACAAGAAAGAGCGAAGAAACAAUCACACCGUGGAACUUCGAAGAAGUCGUCGACAAGGGAGCAAGCGCAC | |
| coding | AGAGCUUCAUCGAAAGAAUGACAAACUUCGACAAGAACCUGCCGAACGAAAAGGUCCUGCCGAAGCACAGCCUGCUGUACGAAUAC | |
| sequence) | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGAC | |
| Amino acid | MDKKYSIGLAIGTNSVGWAVITDEYKVPSKKFKVLGNTDRHSIKKNLIGALLFDSGETAEATRLKRTARRRYTRRKNRICYLQEIF | 219 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| dCas9 | DLNPDNSDVDKLFIQLVQTYNQLFEENPINASGVDAKAILSARLSKSRRLENLIAQLPGEKKNGLFGNLIALSLGLTPNFKSNFDL | |
| (without | AEDAKLQLSKDTYDDDLDNLLAQIGDQYADLFLAAKNLSDAILLSDILRVNTEITKAPLSASMIKRYDEHHQDLTLLKALVRQQLP | |
| NLS) | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDAIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGD | |
| dCas9 mRNA | AUGGACAAGAAGUACAGCAUCGGACUGGCAAUCGGAACAAACAGCGUCGGAUGGGCAGUCAUCACAGACGAAUACAAGGUCCCGAG | 220 |
| ORF encoding | CAAGAAGUUCAAGGUCCUGGGAAACACAGACAGACACAGCAUCAAGAAGAACCUGAUCGGAGCACUGCUGUUCGACAGCGGAGAAA | |
| SEQ ID NO: | CAGCAGAAGCAACAAGACUGAAGAGAACAGCAAGAAGAAGAUACACAAGAAGAAAGAACAGAAUCUGCUACCUGCAGGAAAUCUUC | |
| 19 using | AGCAACGAAAUGGCAAAGGUCGACGACAGCUUCUUCCACAGACUGGAAGAAAGCUUCCUGGUCGAAGAAGACAAGAAGCACGAAAG | |
| minimal | ACACCCGAUCUUCGGAAACAUCGUCGACGAAGUCGCAUACCACGAAAAGUACCCGACAAUCUACCACCUGAGAAAGAAGCUGGUCG | |
| uridine | ACAGCACAGACAAGGCAGACCUGAGACUGAUCUACCUGGCACUGGCACACAUGAUCAAGUUCAGAGGACACUUCCUGAUCGAAGGA | |
| codons as | GACCUGAACCCGGACAACAGCGACGUCGACAAGCUGUUCAUCCAGCUGGUCCAGACAUACAACCAGCUGUUCGAAGAAAACCCGAU | |
| listed in | CAACGCAAGCGGAGUCGACGCAAAGGCAAUCCUGAGCGCAAGACUGAGCAAGAGCAGAAGACUGGAAAACCUGAUCGCACAGCUGC | |
| Table 3, | CGGGAGAAAAGAAGAACGGACUGUUCGGAAACCUGAUCGCACUGAGCCUGGGACUGACACCGAACUUCAAGAGCAACUUCGACCUG | |
| with start | GCAGAAGACGCAAAGCUGCAGCUGAGCAAGGACACAUACGACGACGACCUGGACAACCUGCUGGCACAGAUCGGAGACCAGUACGC | |
| and stop | AGACCUGUUCCUGGCAGCAAAGAACCUGAGCGACGCAAUCCUGCUGAGCGACAUCCUGAGAGUCAACACAGAAAUCACAAAGGCAC | |
| codons | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACGCAAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACUAG | |
| dCas9 coding | GACAAGAAGUACAGCAUCGGACUGGCAAUCGGAACAAACAGCGUCGGAUGGGCAGUCAUCACAGACGAAUACAAGGUCCCGAGCAA | 221 |
| sequence | GAAGUUCAAGGUCCUGGGAAACACAGACAGACACAGCAUCAAGAAGAACCUGAUCGGAGCACUGCUGUUCGACAGCGGAGAAACAG | |
| encoding SEQ | CAGAAGCAACAAGACUGAAGAGAACAGCAAGAAGAAGAUACACAAGAAGAAAGAACAGAAUCUGCUACCUGCAGGAAAUCUUCAGC | |
| ID NO: 19 | AACGAAAUGGCAAAGGUCGACGACAGCUUCUUCCACAGACUGGAAGAAAGCUUCCUGGUCGAAGAAGACAAGAAGCACGAAAGACA | |
| using | CCCGAUCUUCGGAAACAUCGUCGACGAAGUCGCAUACCACGAAAAGUACCCGACAAUCUACCACCUGAGAAAGAAGCUGGUCGACA | |
| minimal | GCACAGACAAGGCAGACCUGAGACUGAUCUACCUGGCACUGGCACACAUGAUCAAGUUCAGAGGACACUUCCUGAUCGAAGGAGAC | |
| uridine | CUGAACCCGGACAACAGCGACGUCGACAAGCUGUUCAUCCAGCUGGUCCAGACAUACAACCAGCUGUUCGAAGAAAACCCGAUCAA | |
| codons as | CGCAAGCGGAGUCGACGCAAAGGCAAUCCUGAGCGCAAGACUGAGCAAGAGCAGAAGACUGGAAAACCUGAUCGCACAGCUGCCGG | |
| listed in | GAGAAAAGAAGAACGGACUGUUCGGAAACCUGAUCGCACUGAGCCUGGGACUGACACCGAACUUCAAGAGCAACUUCGACCUGGCA | |
| Table 3 (no | GAAGACGCAAAGCUGCAGCUGAGCAAGGACACAUACGACGACGACCUGGACAACCUGCUGGCACAGAUCGGAGACCAGUACGCAGA | |
| start or | CCUGUUCCUGGCAGCAAAGAACCUGAGCGACGCAAUCCUGCUGAGCGACAUCCUGAGAGUCAACACAGAAAUCACAAAGGCACCGC | |
| stop codons; | UGAGCGCAAGCAUGAUCAAGAGAUACGACGAACACCACCAGGACCUGACACUGCUGAAGGCACUGGUCAGACAGCAGCUGCCGGAA | |
| suitable for | AAGUACAAGGAAAUCUUCUUCGACCAGAGCAAGAACGGAUACGCAGGAUACAUCGACGGAGGAGCAAGCCAGGAAGAAUUCUACAA | |
| inclusion in | GUUCAUCAAGCCGAUCCUGGAAAAGAUGGACGGAACAGAAGAACUGCUGGUCAAGCUGAACAGAGAAGACCUGCUGAGAAAGCAGA | |
| fusion | GAACAUUCGACAACGGAAGCAUCCCGCACCAGAUCCACCUGGGAGAACUGCACGCAAUCCUGAGAAGACAGGAAGACUUCUACCCG | |
| protein | UUCCUGAAGGACAACAGAGAAAAGAUCGAAAAGAUCCUGACAUUCAGAAUCCCGUACUACGUCGGACCGCUGGCAAGAGGAAACAG | |
| coding | CAGAUUCGCAUGGAUGACAAGAAAGAGCGAAGAAACAAUCACACCGUGGAACUUCGAAGAAGUCGUCGACAAGGGAGCAAGCGCAC | |
| sequence) | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACGCAAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGACGGAGGAGGAAGC | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 222 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| two nuclear | AEDAKLQLSKDTYDDDLDNLLAQIGDQYADLFLAAKNLSDAILLSDILRVNTEITKAPLSASMIKRYDEHHQDLTLLKALVRQQLP | |
| localization | EKYKEIFFDQSKNGYAGYIDGGASQEEFYKFIKPILEKMDGTEELLVKLNREDLLRKQRTFDNGSIPHQIHLGELHAILRRQEDFY | |
| signals as | PFLKDNREKIEKILTFRIPYYVGPLARGNSRFAWMTRKSEETITPWNFEEVVDKGASAQSFIERMTNFDKNLPNEKVLPKHSLLYE | |
| the C- | YFTVYNELTKVKYVTEGMRKPAFLSGEQKKAIVDLLFKTNRKVTVKQLKEDYFKKIECFDSVEISGVEDRFNASLGTYHDLLKIIK | |
| terminal | DKDFLDNEENEDILEDIVLTLTLFEDREMIEERLKTYAHLFDDKVMKQLKRRRYTGWGRLSRKLINGIRDKQSGKTILDFLKSDGF | |
| amino acids | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGD | |
| | GSGSPKKKRKVDGSPKKKRKVDSG | |
| Cas9 mRNA | AUGGACAAGAAGUACAGCAUCGGACUGGACAUCGGAACAAACAGCGUCGGAUGGGCAGUCAUCACAGACGAAUACAAGGUCCCGAG | 223 |
| ORF encoding | CAAGAAGUUCAAGGUCCUGGGAAACACAGACAGACACAGCAUCAAGAAGAACCUGAUCGGAGCACUGCUGUUCGACAGCGGAGAAA | |
| SEQ ID NO: | CAGCAGAAGCAACAAGACUGAAGAGAACAGCAAGAAGAAGAUACACAAGAAGAAAGAACAGAAUCUGCUACCUGCAGGAAAUCUUC | |
| 22 using | AGCAACGAAAUGGCAAAGGUCGACGACAGCUUCUUCCACAGACUGGAAGAAAGCUUCCUGGUCGAAGAAGACAAGAAGCACGAAAG | |
| minimal | ACACCCGAUCUUCGGAAACAUCGUCGACGAAGUCGCAUACCACGAAAAGUACCCGACAAUCUACCACCUGAGAAAGAAGCUGGUCG | |
| uridine | ACAGCACAGACAAGGCAGACCUGAGACUGAUCUACCUGGCACUGGCACACAUGAUCAAGUUCAGAGGACACUUCCUGAUCGAAGGA | |
| codons as | GACCUGAACCCGGACAACAGCGACGUCGACAAGCUGUUCAUCCAGCUGGUCCAGACAUACAACCAGCUGUUCGAAGAAAACCCGAU | |
| listed in | CAACGCAAGCGGAGUCGACGCAAAGGCAAUCCUGAGCGCAAGACUGAGCAAGAGCAGAAGACUGGAAAACCUGAUCGCACAGCUGC | |
| Table 3, | CGGGAGAAAAGAAGAACGGACUGUUCGGAAACCUGAUCGCACUGAGCCUGGGACUGACACCGAACUUCAAGAGCAACUUCGACCUG | |
| with start | GCAGAAGACGCAAAGCUGCAGCUGAGCAAGGACACAUACGACGACGACCUGGACAACCUGCUGGCACAGAUCGGAGACCAGUACGC | |
| and stop | AGACCUGUUCCUGGCAGCAAAGAACCUGAGCGACGCAAUCCUGCUGAGCGACAUCCUGAGAGUCAACACAGAAAUCACAAAGGCAC | |
| codons | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACGGAAGCGGAAGCCCGAAGAAGAAG | |
| | AGAAAGGUCGACGGAAGCCCGAAGAAGAAGAGAAAGGUCGACAGCGGAUAG | |
| Cas9 coding | GACAAGAAGUACAGCAUCGGACUGGACAUCGGAACAAACAGCGUCGGAUGGGCAGUCAUCACAGACGAAUACAAGGUCCCGAGCAA | 224 |
| sequence | GAAGUUCAAGGUCCUGGGAAACACAGACAGACACAGCAUCAAGAAGAACCUGAUCGGAGCACUGCUGUUCGACAGCGGAGAAACAG | |
| encoding SEQ | CAGAAGCAACAAGACUGAAGAGAACAGCAAGAAGAAGAUACACAAGAAGAAAGAACAGAAUCUGCUACCUGCAGGAAAUCUUCAGC | |
| ID NO: 23 | AACGAAAUGGCAAAGGUCGACGACAGCUUCUUCCACAGACUGGAAGAAAGCUUCCUGGUCGAAGAAGACAAGAAGCACGAAAGACA | |
| using | CCCGAUCUUCGGAAACAUCGUCGACGAAGUCGCAUACCACGAAAAGUACCCGACAAUCUACCACCUGAGAAAGAAGCUGGUCGACA | |
| minimal | GCACAGACAAGGCAGACCUGAGACUGAUCUACCUGGCACUGGCACACAUGAUCAAGUUCAGAGGACACUUCCUGAUCGAAGGAGAC | |
| uridine | CUGAACCCGGACAACAGCGACGUCGACAAGCUGUUCAUCCAGCUGGUCCAGACAUACAACCAGCUGUUCGAAGAAAACCCGAUCAA | |
| codons as | CGCAAGCGGAGUCGACGCAAAGGCAAUCCUGAGCGCAAGACUGAGCAAGAGCAGAAGACUGGAAAACCUGAUCGCACAGCUGCCGG | |
| listed in | GAGAAAAGAAGAACGGACUGUUCGGAAACCUGAUCGCACUGAGCCUGGGACUGACACCGAACUUCAAGAGCAACUUCGACCUGGCA | |
| Table 3 (no | GAAGACGCAAAGCUGCAGCUGAGCAAGGACACAUACGACGACGACCUGGACAACCUGCUGGCACAGAUCGGAGACCAGUACGCAGA | |
| start or | CCUGUUCCUGGCAGCAAAGAACCUGAGCGACGCAAUCCUGCUGAGCGACAUCCUGAGAGUCAACACAGAAAUCACAAAGGCACCGC | |
| stop codons; | UGAGCGCAAGCAUGAUCAAGAGAUACGACGAACACCACCAGGACCUGACACUGCUGAAGGCACUGGUCAGACAGCAGCUGCCGGAA | |
| suitable for | AAGUACAAGGAAAUCUUCUUCGACCAGAGCAAGAACGGAUACGCAGGAUACAUCGACGGAGGAGCAAGCCAGGAAGAAUUCUACAA | |
| inclusion in | GUUCAUCAAGCCGAUCCUGGAAAAGAUGGACGGAAGAGAAGAACUGCUGGUCAAGCUGAACAGAGAAGACCUGCUGAGAAAGCAGA | |
| fusion | GAACAUUCGACAACGGAAGCAUCCCGCACCAGAUCCACCUGGGAGAACUGCACGCAAUCCUGAGAAGACAGGAAGACUUCUACCCG | |
| protein | UUCCUGAAGGACAACAGAGAAAAGAUCGAAAAGAUCCUGACAUUCAGAAUCCCGUACUACGUCGGACCGCUGGCAAGAGGAAACAG | |
| coding | CAGAUUCGCAUGGAUGACAAGAAAGAGCGAAGAAACAAUCACACCGUGGAACUUCGAAGAAGUCGUCGACAAGGGAGCAAGCGCAC | |
| sequence) | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGACGGAAGCGGAAGCCCGAAGAAGAAGAGA | |
| | AAGGUCGACGGAAGCCCGAAGAAGAAGAGAAAGGUCGACAGCGGA | |
| Amino acid | MDKKYSIGLAIGTNSVGWAVITDEYKVPSKKFKVLGNTDRHSIKKNLIGALLFDSGETAEATRLKRTARRRYTRRKNRICYLQEIF | 225 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 nickase | DLNPDNSDVDKLFIQLVQTYNQLFEENPINASGVDAKAILSARLSKSRRLENLIAQLPGEKKNGLFGNLIALSLGLTPNFKSNFDL | |
| with two | AEDAKLQLSKDTYDDDLDNLLAQIGDQYADLFLAAKNLSDAILLSDILRVNTEITKAPLSASMIKRYDEHHQDLTLLKALVRQQLP | |
| nuclear | EKYKEIFFDQSKNGYAGYIDGGASQEEFYKFIKPILEKMDGTEELLVKLNREDLLRKQRTFDNGSIPHQIHLGELHAILRRQEDFY | |
| localization | PFLKDNREKIEKILTFRIPYYVGPLARGNSRFAWMTRKSEETITPWNFEEVVDKGASAQSFIERMTNFDKNLPNEKVLPKHSLLYE | |
| signals as | YFTVYNELTKVKYVTEGMRKPAFLSGEQKKAIVDLLFKTNRKVTVKQLKEDYFKKIECFDSVEISGVEDRFNASLGTYHDLLKIIK | |
| the C- | DKDFLDNEENEDILEDIVLTLTLFEDREMIEERLKTYAHLFDDKVMKQLKRRRYTGWGRLSRKLINGIRDKQSGKTILDFLKSDGF | |
| terminal | ANRNFMQLIHDDSLTFKEDIQKAQVSGQGDSLHEHIANLAGSPAIKKGILQTVKVVDELVKVMGRHKPENIVIEMARENQTTQKGQ | |
| amino acids | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGSGSPKKK | |
| | RKVDGSPKKKRKVDSG | |
| Cas9 nickase | AUGGACAAGAAGUACAGCAUCGGACUGGCAAUCGGAACAAACAGCGUCGGAUGGGCAGUCAUCACAGACGAAUACAAGGUCCCGAG | 226 |
| mRNA ORF | CAAGAAGUUCAAGGUCCUGGGAAACACAGACAGACACAGCAUCAAGAAGAACCUGAUCGGAGCACUGCUGUUCGACAGCGGAGAAA | |
| encoding SEQ | CAGCAGAAGCAACAAGACUGAAGAGAACAGCAAGAAGAAGAUACACAAGAAGAAAGAACAGAAUCUGCUACCUGCAGGAAAUCUUC | |
| ID NO: 25 | AGCAACGAAAUGGCAAAGGUCGACGACAGCUUCUUCCACAGACUGGAAGAAAGCUUCCUGGUCGAAGAAGACAAGAAGCACGAAAG | |
| using | ACACCCGAUCUUCGGAAACAUCGUCGACGAAGUCGCAUACCACGAAAAGUACCCGACAAUCUACCACCUGAGAAAGAAGCUGGUCG | |
| minimal | ACAGCACAGACAAGGCAGACCUGAGACUGAUCUACCUGGCACUGGCACACAUGAUCAAGUUCAGAGGACACUUCCUGAUCGAAGGA | |
| uridine | GACCUGAACCCGGACAACAGCGACGUCGACAAGCUGUUCAUCCAGCUGGUCCAGACAUACAACCAGCUGUUCGAAGAAAACCCGAU | |
| codons as | CAACGCAAGCGGAGUCGACGCAAAGGCAAUCCUGAGCGCAAGACUGAGCAAGAGCAGAAGACUGGAAAACCUGAUCGCACAGCUGC | |
| listed in | CGGGAGAAAAGAAGAACGGACUGUUCGGAAACCUGAUCGCACUGAGCCUGGGACUGACACCGAACUUCAAGAGCAACUUCGACCUG | |
| Table 3, | GCAGAAGACGCAAAGCUGCAGCUGAGCAAGGACACAUACGACGACGACCUGGACAACCUGCUGGCACAGAUCGGAGACCAGUACGC | |
| with start | AGACCUGUUCCUGGCAGCAAAGAACCUGAGCGACGCAAUCCUGCUGAGCGACAUCCUGAGAGUCAACACAGAAAUCACAAAGGCAC | |
| and stop | CGCUGAGCGCAAGCAUGAUCAAGAGAUACGACGAACACCACCAGGACCUGACACUGCUGAAGGCACUGGUCAGACAGCAGCUGCCG | |
| codons | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACCACAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGACGGAAGCGGAAGCCCGAAGAAGAAG | |
| | AGAAAGGUCGACGGAAGCCCGAAGAAGAAGAGAAAGGUCGACAGCGGAUAG | |
| Cas9 nickase | GACAAGAAGUACAGCAUCGGACUGGCAAUCGGAACAAACAGCGUCGGAUGGGCAGUCAUCACAGACGAAUACAAGGUCCCGAGCAA | 227 |
| coding | GAAGUUCAAGGUCCUGGGAAACACAGACAGACACAGCAUCAAGAAGAACCUGAUCGGAGCACUGCUGUUCGACAGCGGAGAAACAG | |
| sequence | CAGAAGCAACAAGACUGAAGAGAACAGCAAGAAGAAGAUACACAAGAAGAAAGAACAGAAUCUGCUACCUGCAGGAAAUCUUCAGC | |
| encoding SEQ | AACGAAAUGGCAAAGGUCGACGACAGCUUCUUCCACAGACUGGAAGAAAGCUUCCUGGUCGAAGAAGACAAGAAGCACGAAAGACA | |
| ID NO: 25 | CCCGAUCUUCGGAAACAUCGUCGACGAAGUCGCAUACCACGAAAAGUACCCGACAAUCUACCACCUGAGAAAGAAGCUGGUCGACA | |
| using | GCACAGACAAGGCAGACCUGAGACUGAUCUACCUGGCACUGGCACACAUGAUCAAGUUCAGAGGACACUUCCUGAUCGAAGGAGAC | |
| minimal | CUGAACCCGGACAACAGCGACGUCGACAAGCUGUUCAUCCAGCUGGUCCAGACAUACAACCAGCUGUUCGAAGAAAACCCGAUCAA | |
| uridine | CGCAAGCGGAGUCGACGCAAAGGCAAUCCUGAGCGCAAGACUGAGCAAGAGCAGAAGACUGGAAAACCUGAUCGCACAGCUGCCGG | |
| codons as | GAGAAAAGAAGAACGGACUGUUCGGAAACCUGAUCGCACUGAGCCUGGGACUGACACCGAACUUCAAGAGCAACUUCGACCUGGCA | |
| listed in | GAAGACGCAAAGCUGCAGCUGAGCAAGGACACAUACGACGACGACCUGGACAACCUGCUGGCACAGAUCGGAGACCAGUACGCAGA | |
| Table 3 (no | CCUGUUCCUGGCAGCAAAGAACCUGAGCGACGCAAUCCUGCUGAGCGACAUCCUGAGAGUCAACACAGAAAUCACAAAGGCACCGC | |
| start or | UGAGCGCAAGCAUGAUCAAGAGAUACGACGAACACCACCAGGACCUGACACUGCUGAAGGCACUGGUCAGACAGCAGCUGCCGGAA | |
| stop codons; | AAGUACAAGGAAAUCUUCUUCGACCAGAGCAAGAACGGAUACGCAGGAUACAUCGACGGAGGAGCAAGCCAGGAAGAAUUCUACAA | |
| suitable for | GUUCAUCAAGCCGAUCCUGGAAAAGAUGGACGGAACAGAAGAACUGCUGGUCAAGCUGAACAGAGAAGACCUGCUGAGAAAGCAGA | |
| inclusion in | GAACAUUCGACAACGGAAGCAUCCCGCACCAGAUCCACCUGGGAGAACUGCACGCAAUCCUGAGAAGACAGGAAGACUUCUACCCG | |
| fusion | UUCCUGAAGGACAACAGAGAAAAGAUCGAAAAGAUCCUGACAUUCAGAAUCCCGUACUACGUCGGACCGCUGGCAAGAGGAAACAG | |
| protein | CAGAUUCGCAUGGAUGACAAGAAAGAGCGAAGAAACAAUCACACCGUGGAACUUCGAAGAAGUCGUCGACAAGGGAGCAAGCGCAC | |
| coding | AGAGCUUCAUCGAAAGAAUGACAAACUUCGACAAGAACCUGCCGAACGAAAAGGUCCUGCCGAAGCACAGCCUGCUGUACGAAUAC | |
| sequence) | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACCACAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGAC | |
| | GGAAGCGGAAGCCCGAAGAAGAAGAGAAAGGUCGACGGAAGCCCGAAGAAGAAGAGAAAGGUCGACAGCGGA | |
| Amino acid | MDKKYSIGLAIGTNSVGWAVITDEYKVPSKKFKVLGNTDRHSIKKNLIGALLFDSGETAEATRLKRTARRRYTRRKNRICYLQEIF | 228 |
| sequence of | SNEMAKVDDSFFHRLEESFLVEEDKKHERHPIFGNIVDEVAYHEKYPTIYHLRKKLVDSTDKADLRLIYLALAHMIKFRGHFLIEG | |
| dCas9 with | DLNPDNSDVDKLFIQLVQTYNQLFEENPINASGVDAKAILSARLSKSRRLENLIAQLPGEKKNGLFGNLIALSLGLTPNFKSNFDL | |
| two nuclear | AEDAKLQLSKDTYDDDLDNLLAQIGDQYADLFLAAKNLSDAILLSDILRVNTEITKAPLSASMIKRYDEHHQDLTLLKALVRQQLP | |
| localization | EKYKEIFFDQSKNGYAGYIDGGASQEEFYKFIKPILEKMDGTEELLVKLNREDLLRKQRTFDNGSIPHQIHLGELHAILRRQEDFY | |
| signals as | PFLKDNREKIEKILTFRIPYYVGPLARGNSRFAWMTRKSEETITPWNFEEVVDKGASAQSFIERMTNFDKNLPNEKVLPKHSLLYE | |
| the C-terminal | YFTVYNELTKVKYVTEGMRKPAFLSGEQKKAIVDLLFKTNRKVTVKQLKEDYFKKIECFDSVEISGVEDRFNASLGTYHDLLKIIK | |
| amino acids | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDAIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGSGSPKKK | |
| | RKVDGSPKKKRKVDSG | |
| dCas9 mRNA | AUGGACAAGAAGUACAGCAUCGGACUGGCAAUCGGAACAAACAGCGUCGGAUGGGCAGUCAUCACAGACGAAUACAAGGUCCCGAG | 229 |
| ORF encoding | CAAGAAGUUCAAGGUCCUGGGAAACACAGACAGACACAGCAUCAAGAAGAACCUGAUCGGAGCACUGCUGUUCGACAGCGGAGAAA | |
| SEQ ID NO: | CAGCAGAAGCAACAAGACUGAAGAGAACAGCAAGAAGAAGAUACACAAGAAGAAAGAACAGAAUCUGCUACCUGCAGGAAAUCUUC | |
| 28 using | AGCAACGAAAUGGCAAAGGUCGACGACAGCUUCUUCCACAGACUGGAAGAAAGCUUCCUGGUCGAAGAAGACAAGAAGCACGAAAG | |
| minimal | ACACCCGAUCUUCGGAAACAUCGUCGACGAAGUCGCAUACCACGAAAAGUACCCGACAAUCUACCACCUGAGAAAGAAGCUGGUCG | |
| uridine | ACAGCACAGACAAGGCAGACCUGAGACUGAUCUACCUGGCACUGGCACACAUGAUCAAGUUCAGAGGACACUUCCUGAUCGAAGGA | |
| codons as | GACCUGAACCCGGACAACAGCGACGUCGACAAGCUGUUCAUCCAGCUGGUCCAGACAUACAACCAGCUGUUCGAAGAAAACCCGAU | |
| listed in | CAACGCAAGCGGAGUCGACGCAAAGGCAAUCCUGAGCGCAAGACUGAGCAAGAGCAGAAGACUGGAAAACCUGAUCGCACAGCUGC | |
| Table 3, | CGGGAGAAAAGAAGAACGGACUGUUCGGAAACCUGAUCGCACUGAGCCUGGGACUGACACCGAACUUCAAGAGCAACUUCGACCUG | |
| with start | GCAGAAGACGCAAAGCUGCAGCUGAGCAAGGACACAUACGACGACGACCUGGACAACCUGCUGGCACAGAUCGGAGACCAGUACGC | |
| and stop | AGACCUGUUCCUGGCAGCAAAGAACCUGAGCGACGCAAUCCUGCUGAGCGACAUCCUGAGAGUCAACACAGAAAUCACAAAGGCAC | |
| codons | CGCUGAGCGCAAGCAUGAUCAAGAGAUACGACGAACACCACCAGGACCUGACACUGCUGAAGGCACUGGUCAGACAGCAGCUGCCG | |
| | GAAAAGUACAAGGAAAUCUUCUUCGACCAGAGCAAGAACGGAUACGCAGGAUACAUCGACGGAGGAGCAAGCCAGGAAGAAUUCUA | |
| | CAAGUUCAUCAAGCCGAUCCUGGAAAAGAUGGACGGAACAGAAGAACUGCUGGUCAAGCUGAACAGAGAAGACCUGCUGAGAAAGC | |
| | AGAGAACAUUCGACAACGGAAGCAUCCCGCACCAGAUCCACCUGGGAGAACUGCACGCAAUCCUGAGAAGACAGGAAGACUUCUAC | |
| | CCGUUCCUGAAGGACAACAGAGAAAAGAUCGAAAAGAUCCUGACAUUCAGAAUCCCGUACUACGUCGGACCGCUGGCAAGAGGAAA | |
| | CAGCAGAUUCGCAUGGAUGACAAGAAAGAGCGAAGAAACAAUCACACCGUGGAACUUCGAAGAAGUCGUCGACAAGGGAGCAAGCG | |
| | CACAGAGCUUCAUCGAAAGAAUGACAAACUUCGACAAGAACCUGCCGAACGAAAAGGUCCUGCCGAAGCACAGCCUGCUGUACGAA | |
| | UACUUCACAGUCUACAACGAACUGACAAAGGUCAAGUACGUCACAGAAGGAAUGAGAAAGCCGGCAUUCCUGAGCGGAGAACAGAA | |
| | GAAGGCAAUCGUCGACCUGCUGUUCAAGACAAACAGAAAGGUCACAGUCAAGCAGCUGAAGGAAGACUACUUCAAGAAGAUCGAAU | |
| | GCUUCGACAGCGUCGAAAUCAGCGGAGUCGAAGACAGAUUCAACGCAAGCCUGGGAACAUACCACGACCUGCUGAAGAUCAUCAAG | |
| | GACAAGGACUUCCUGGACAACGAAGAAAACGAAGACAUCCUGGAAGACAUCGUCCUGACACUGACACUGUUCGAAGACAGAGAAAU | |
| | GAUCGAAGAAAGACUGAAGACAUACGCACACCUGUUCGACGACAAGGUCAUGAAGCAGCUGAAGAGAAGAAGAUACACAGGAUGGG | |
| | GAAGACUGAGCAGAAAGCUGAUCAACGGAAUCAGAGACAAGCAGAGCGGAAAGACAAUCCUGGACUUCCUGAAGAGCGACGGAUUC | |
| | GCAAACAGAAACUUCAUGCAGCUGAUCCACGACGACAGCCUGACAUUCAAGGAAGACAUCCAGAAGGCACAGGUCAGCGGACAGGG | |
| | AGACAGCCUGCACGAACACAUCGCAAACCUGGCAGGAAGCCCGGCAAUCAAGAAGGGAAUCCUGCAGACAGUCAAGGUCGUCGACG | |
| | AACUGGUCAAGGUCAUGGGAAGACACAAGCCGGAAAACAUCGUCAUCGAAAUGGCAAGAGAAAACCAGACAACACAGAAGGGACAG | |
| | AAGAACAGCAGAGAAAGAAUGAAGAGAAUCGAAGAAGGAAUCAAGGAACUGGGAAGCCAGAUCCUGAAGGAACACCCGGUCGAAAA | |
| | CACACAGCUGCAGAACGAAAAGCUGUACCUGUACUACCUGCAGAACGGAAGAGACAUGUACGUCGACCAGGAACUGGACAUCAACA | |
| | GACUGAGCGACUACGACGUCGACGCAAUCGUCCCGCAGAGCUUCCUGAAGGACGACAGCAUCGACAACAAGGUCCUGACAAGAAGC | |
| | GACAAGAACAGAGGAAAGAGCGACAACGUCCCGAGCGAAGAAGUCGUCAAGAAGAUGAAGAACUACUGGAGACAGCUGCUGAACGC | |
| | AAAGCUGAUCACACAGAGAAAGUUCGACAACCUGACAAAGGCAGAGAGAGGAGGACUGAGCGAACUGGACAAGGCAGGAUUCAUCA | |
| | AGAGACAGCUGGUCGAAACAAGACAGAUCACAAAGCACGUCGCACAGAUCCUGGACAGCAGAAUGAACACAAAGUACGACGAAAAC | |
| | GACAAGCUGAUCAGAGAAGUCAAGGUCAUCACACUGAAGAGCAAGCUGGUCAGCGACUUCAGAAAGGACUUCCAGUUCUACAAGGU | |
| | CAGAGAAAUCAACAACUACCACCACGCACACGACGCAUACCUGAACGCAGUCGUCGGAACAGCACUGAUCAAGAAGUACCCGAAGC | |
| | UGGAAAGCGAAUUCGUCUACGGAGACUACAAGGUCUACGACGUCAGAAAGAUGAUCGCAAAGAGCGAACAGGAAAUCGGAAAGGCA | |
| | ACAGCAAAGUACUUCUUCUACAGCAACAUCAUGAACUUCUUCAAGACAGAAAUCACACUGGCAAACGGAGAAAUCAGAAAGAGACC | |
| | GCUGAUCGAAACAAACGGAGAAACAGGAGAAAUCGUCUGGGACAAGGGAAGAGACUUCGCAACAGUCAGAAAGGUCCUGAGCAUGC | |
| | CGCAGGUCAACAUCGUCAAGAAGACAGAAGUCCAGACAGGAGGAUUCAGCAAGGAAAGCAUCCUGCCGAAGAGAAACAGCGACAAG | |
| | CUGAUCGCAAGAAAGAAGGACUGGGACCCGAAGAAGUACGGAGGAUUCGACAGCCCGACAGUCGCAUACAGCGUCCUGGUCGUCGC | |
| | AAAGGUCGAAAAGGGAAAGAGCAAGAAGCUGAAGAGCGUCAAGGAACUGCUGGGAAUCACAAUCAUGGAAAGAAGCAGCUUCGAAA | |
| | AGAACCCGAUCGACUUCCUGGAAGCAAAGGGAUACAAGGAAGUCAAGAAGGACCUGAUCAUCAAGCUGCCGAAGUACAGCCUGUUC | |
| | GAACUGGAAAACGGAAGAAAGAGAAUGCUGGCAAGCGCAGGAGAACUGCAGAAGGGAAACGAACUGGCACUGCCGAGCAAGUACGU | |
| | CAACUUCCUGUACCUGGCAAGCCACUACGAAAAGCUGAAGGGAAGCCCGGAAGACAACGAACAGAAGCAGCUGUUCGUCGAACAGC | |
| | ACAAGCACUACCUGGACGAAAUCAUCGAACAGAUCAGCGAAUUCAGCAAGAGAGUCAUCCUGGCAGACGCAAACCUGGACAAGGUC | |
| | CUGAGCGCAUACAACAAGCACAGAGACAAGCCGAUCAGAGAACAGGCAGAAAACAUCAUCCACCUGUUCACACUGACAAACCUGGG | |
| | AGCACCGGCAGCAUUCAAGUACUUCGACACAACAAUCGACAGAAAGAGAUACACAAGCACAAAGGAAGUCCUGGACGCAACACUGA | |
| | UCCACCAGAGCAUCACAGGACUGUACGAAACAAGAAUCGACCUGAGCCAGCUGGGAGGAGAC | |
| | GGAAGCGGAAGCCCGAAGAAGAAGAGAAAGGUCGACGGAAGCCCGAAGAAGAAGAGAAAGGUCGACAGCGGAUAG | |
| dCas9 coding | GACAAGAAGUACAGCAUCGGACUGGCAAUCGGAACAAACAGCGUCGGAUGGGCAGUCAUCACAGACGAAUACAAGGUCCCGAGCAA | 230 |
| sequence | GAAGUUCAAGGUCCUGGGAAACACAGACAGACACAGCAUCAAGAAGAACCUGAUCGGAGCACUGCUGUUCGACAGCGGAGAAACAG | |
| encoding SEQ | CAGAAGCAACAAGACUGAAGAGAACAGCAAGAAGAAGAUACACAAGAAGAAAGAACAGAAUCUGCUACCUGCAGGAAAUCUUCAGC | |
| ID NO: 28 | AACGAAAUGGCAAAGGUCGACGACAGCUUCUUCCACAGACUGGAAGAAAGCUUCCUGGUCGAAGAAGACAAGAAGCACGAAAGACA | |
| using | CCCGAUCUUCGGAAACAUCGUCGACGAAGUCGCAUACCACGAAAAGUACCCGACAAUCUACCACCUGAGAAAGAAGCUGGUCGACA | |
| minimal | GCACAGACAAGGCAGACCUGAGACUGAUCUACCUGGCACUGGCACACAUGAUCAAGUUCAGAGGACACUUCCUGAUCGAAGGAGAC | |
| uridine | CUGAACCCGGACAACAGCGACGUCGACAAGCUGUUCAUCCAGCUGGUCCAGACAUACAACCAGCUGUUCGAAGAAAACCCGAUCAA | |
| codons as | CGCAAGCGGAGUCGACGCAAAGGCAAUCCUGAGCGCAAGACUGAGCAAGAGCAGAAGACUGGAAAACCUGAUCGCACAGCUGCCGG | |
| listed in | GAGAAAAGAAGAACGGACUGUUCGGAAACCUGAUCGCACUGAGCCUGGGACUGACACCGAACUUCAAGAGCAACUUCGACCUGGCA | |
| Table 3 (no | GAAGACGCAAAGCUGCAGCUGAGCAAGGACACAUACGACGACGACCUGGACAACCUGCUGGCACAGAUCGGAGACCAGUACGCAGA | |
| start or | CCUGUUCCUGGCAGCAAAGAACCUGAGCGACGCAAUCCUGCUGAGCGACAUCCUGAGAGUCAACACAGAAAUCACAAAGGCACCGC | |
| stop codons; | UGAGCGCAAGCAUGAUCAAGAGAUACGACGAACACCACCAGGACCUGACACUGCUGAAGGCACUGGUCAGACAGCAGCUGCCGGAA | |
| suitable for | AAGUACAAGGAAAUCUUCUUCGACCAGAGCAAGAACGGAUACGCAGGAUACAUCGACGGAGGAGCAAGCCAGGAAGAAUUCUACAA | |
| inclusion in | GUUCAUCAAGCCGAUCCUGGAAAAGAUGGACGGAAGAGAAGAACUGCUGGUCAAGCUGAACAGAGAAGACCUGCUGAGAAAGCAGA | |
| fusion | GAACAUUCGACAACGGAAGCAUCCCGCACCAGAUCCACCUGGGAGAACUGCACGCAAUCCUGAGAAGACAGGAAGACUUCUACCCG | |
| protein | UUCCUGAAGGACAACAGAGAAAAGAUCGAAAAGAUCCUGACAUUCAGAAUCCCGUACUACGUCGGACCGCUGGCAAGAGGAAACAG | |
| coding | CAGAUUCGCAUGGAUGACAAGAAAGAGCGAAGAAACAAUCACACCGUGGAACUUCGAAGAAGUCGUCGACAAGGGAGCAAGCGCAC | |
| sequence) | AGAGCUUCAUCGAAAGAAUGACAAACUUCGACAAGAACCUGCCGAACGAAAAGGUCCUGCCGAAGCACAGCCUGCUGUACGAAUAC | |
| | UUCACAGUCUACAACGAACUGACAAAGGUCAAGUACGUCACAGAAGGAAUGAGAAAGCCGGCAUUCCUGAGCGGAGAACAGAAGAA | |
| | GGCAAUCGUCGACCUGCUGUUCAAGACAAACAGAAAGGUCACAGUCAAGCAGCUGAAGGAAGACUACUUCAAGAAGAUCGAAUGCU | |
| | UCGACAGCGUCGAAAUCAGCGGAGUCGAAGACAGAUUCAACGCAAGCCUGGGAACAUACCACGACCUGCUGAAGAUCAUCAAGGAC | |
| | AAGGACUUCCUGGACAACGAAGAAAACGAAGACAUCCUGGAAGACAUCGUCCUGACACUGACACUGUUCGAAGACAGAGAAAUGAU | |
| | CGAAGAAAGACUGAAGACAUACGCACACCUGUUCGACGACAAGGUCAUGAAGCAGCUGAAGAGAAGAAGAUACACAGGAUGGGGAA | |
| | GACUGAGCAGAAAGCUGAUCAACGGAAUCAGAGACAAGCAGAGCGGAAAGACAAUCCUGGACUUCCUGAAGAGCGACGGAUUCGCA | |
| | AACAGAAACUUCAUGCAGCUGAUCCACGACGACAGCCUGACAUUCAAGGAAGACAUCCAGAAGGCACAGGUCAGCGGACAGGGAGA | |
| | CAGCCUGCACGAACACAUCGCAAACCUGGCAGGAAGCCCGGCAAUCAAGAAGGGAAUCCUGCAGACAGUCAAGGUCGUCGACGAAC | |
| | UGGUCAAGGUCAUGGGAAGACACAAGCCGGAAAACAUCGUCAUCGAAAUGGCAAGAGAAAACCAGACAACACAGAAGGGACAGAAG | |
| | AACAGCAGAGAAAGAAUGAAGAGAAUCGAAGAAGGAAUCAAGGAACUGGGAAGCCAGAUCCUGAAGGAACACCCGGUCGAAAACAC | |
| | ACAGCUGCAGAACGAAAAGCUGUACCUGUACUACCUGCAGAACGGAAGAGACAUGUACGUCGACCAGGAACUGGACAUCAACAGAC | |
| | UGAGCGACUACGACGUCGACGCAAUCGUCCCGCAGAGCUUCCUGAAGGACGACAGCAUCGACAACAAGGUCCUGACAAGAAGCGAC | |
| | AAGAACAGAGGAAAGAGCGACAACGUCCCGAGCGAAGAAGUCGUCAAGAAGAUGAAGAACUACUGGAGACAGCUGCUGAACGCAAA | |
| | GCUGAUCACACAGAGAAAGUUCGACAACCUGACAAAGGCAGAGAGAGGAGGACUGAGCGAACUGGACAAGGCAGGAUUCAUCAAGA | |
| | GACAGCUGGUCGAAACAAGACAGAUCACAAAGCACGUCGCACAGAUCCUGGACAGCAGAAUGAACACAAAGUACGACGAAAACGAC | |
| | AAGCUGAUCAGAGAAGUCAAGGUCAUCACACUGAAGAGCAAGCUGGUCAGCGACUUCAGAAAGGACUUCCAGUUCUACAAGGUCAG | |
| | AGAAAUCAACAACUACCACCACGCACACGACGCAUACCUGAACGCAGUCGUCGGAACAGCACUGAUCAAGAAGUACCCGAAGCUGG | |
| | AAAGCGAAUUCGUCUACGGAGACUACAAGGUCUACGACGUCAGAAAGAUGAUCGCAAAGAGCGAACAGGAAAUCGGAAAGGCAACA | |
| | GCAAAGUACUUCUUCUACAGCAACAUCAUGAACUUCUUCAAGACAGAAAUCACACUGGCAAACGGAGAAAUCAGAAAGAGACCGCU | |
| | GAUCGAAACAAACGGAGAAACAGGAGAAAUCGUCUGGGACAAGGGAAGAGACUUCGCAACAGUCAGAAAGGUCCUGAGCAUGCCGC | |
| | AGGUCAACAUCGUCAAGAAGACAGAAGUCCAGACAGGAGGAUUCAGCAAGGAAAGCAUCCUGCCGAAGAGAAACAGCGACAAGCUG | |
| | AUCGCAAGAAAGAAGGACUGGGACCCGAAGAAGUACGGAGGAUUCGACAGCCCGACAGUCGCAUACAGCGUCCUGGUCGUCGCAAA | |
| | GGUCGAAAAGGGAAAGAGCAAGAAGCUGAAGAGCGUCAAGGAACUGCUGGGAAUCACAAUCAUGGAAAGAAGCAGCUUCGAAAAGA | |
| | ACCCGAUCGACUUCCUGGAAGCAAAGGGAUACAAGGAAGUCAAGAAGGACCUGAUCAUCAAGCUGCCGAAGUACAGCCUGUUCGAA | |
| | CUGGAAAACGGAAGAAAGAGAAUGCUGGCAAGCGCAGGAGAACUGCAGAAGGGAAACGAACUGGCACUGCCGAGCAAGUACGUCAA | |
| | CUUCCUGUACCUGGCAAGCCACUACGAAAAGCUGAAGGGAAGCCCGGAAGACAACGAACAGAAGCAGCUGUUCGUCGAACAGCACA | |
| | AGCACUACCUGGACGAAAUCAUCGAACAGAUCAGCGAAUUCAGCAAGAGAGUCAUCCUGGCAGACGCAAACCUGGACAAGGUCCUG | |
| | AGCGCAUACAACAAGCACAGAGACAAGCCGAUCAGAGAACAGGCAGAAAACAUCAUCCACCUGUUCACACUGACAAACCUGGGAGC | |
| | ACCGGCAGCAUUCAAGUACUUCGACACAACAAUCGACAGAAAGAGAUACACAAGCACAAAGGAAGUCCUGGACGCAACACUGAUCC | |
| | ACCAGAGCAUCACAGGACUGUACGAAACAAGAAUCGACCUGAGCCAGCUGGGAGGAGAC | |
| | GGAAGCGGAAGCCCGAAGAAGAAGAGAAAGGUCGACGGAAGCCCGAAGAAGAAGAGAAAGGUCGACAGCGGA | |
| T7 promoter | TAATACGACTCACTATA | 231 |
| Human beta- | ACATTTGCTTCTGACACAACTGTGTTCACTAGCAACCTCAAACAGACACC | 232 |
| globin 5′ | | |
| UTR | | |
| Human beta- | GCTCGCTTTCTTGCTGTCCAATTTCTATTAAAGGTTCCTTTGTTCCCTAAGTCCAACTACTAAACTGGGGGATATTATGAAGGGCC | 233 |
| globin 3′ | TTGAGCATCTGGATTCTGCCTAATAAAAAACATTTATTTTCATTGC | |
| UTR | | |
| Human alpha- | CATAAACCCTGGCGCGCTCGCGGCCCGGCACTCTTCTGGTCCCCACAGACTCAGAGAGAACCCACC | 234 |
| globin 5′ | | |
| UTR | | |
| Human alpha- | GCTGGAGCCTCGGTGGCCATGCTTCTTGCCCCTTGGGCCTCCCCCCAGCCCCTCCTCCCCTTCCTGCACCCGTACCCCCGTGGTCT | 235 |
| globin 3′ | TTGAATAAAGTCTGAGTGGGCGGC | |
| UTR | | |
| Xenopus | AAGCTCAGAATAAACGCTCAACTTTGGCC | 236 |
| laevis beta- | | |
| globin 5′ | | |
| UTR | | |
| Xenopus | ACCAGCCTCAAGAACACCCGAATGGAGTCTCTAAGCTACATAATACCAACTTACACTTTACAAAATGTTGTCCCCCAAAATGTAGC | 237 |
| laevis beta- | CATTCGTATCTGCTCCTAATAAAAAGAAAGTTTCTTCACATTCT | |
| globin 3′ | | |
| UTR | | |
| Bovine | CAGGGTCCTGTGGACAGCTCACCAGCT | 238 |
| Growth | | |
| Hormone 5′ | | |
| UTR | | |
| Bovine | TTGCCAGCCATCTGTTGTTTGCCCCTCCCCCGTGCCTTCCTTGACCCTGGAAGGTGCCACTCCCACTGTCCTTTCCTAATAAAATG | 239 |
| Growth | AGGAAATTGCATCGCA | |
| Hormone 3′ | | |
| UTR | | |
| Mus musculus | GCTGCCTTCTGCGGGGCTTGCCTTCTGGCCATGCCCTTCTTCTCTCCCTTGCACCTGTACCTCTTGGTCTTTGAATAAAGCCTGAG | 240 |
| hemoglobin | TAGGAAG | |
| alpha, adult | | |
| chain 1 | | |
| (Hba-a1), | | |
| 3′UTR | | |
| HSD17B4 5′ | TCCCGCAGTCGGCGTCCAGCGGCTCTGCTTGTTCGTGTGTGTGTCGTTGCAGGCCTTATTC | 241 |
| UTR | | |
| G282 guide | mU*mU*mA*CAGCCACGUCUACAGCAGUUUUAGAmGmCmUmAmGmAmAmAmUmAmGmCAAGUUAAAAUAAGGCUAGUCCGUUAUCA | 242 |
| RNA | mAmCmUmUmGGmUmGmGmCmAmCmCmGmAmGmUmCmGmGmUmGmCmU*mU*mU*mU | |
| targeting | | |
| TTR | | |
| Cas9 | GGGTCCCGCAGTCGGCGTCCAGCGGCTCTGCTTGTTCGTGTGTGTGTCGTTGCAGGCCTTATTCGGATCCGCCACCATGGACAAGA | 243 |
| transcript | AGTACAGCATCGGACTGGACATCGGAACAAACAGCGTCGGATGGGCAGTCATCACAGACGAATACAAGGTCCCGAGCAAGAAGTTC | |
| with 5′ UTR | AAGGTCCTGGGAAACACAGACAGACACAGCATCAAGAAGAACCTGATCGGAGCACTGCTGTTCGACAGCGGAGAAACAGCAGAAGC | |
| of HSD, ORF | AACAAGACTGAAGAGAACAGCAAGAAGAAGATACACAAGAAGAAAGAACAGAATCTGCTACCTGCAGGAAATCTTCAGCAACGAAA | |
| corresponding | TGGCAAAGGTCGACGACAGCTTCTTCCACAGACTGGAAGAAAGCTTCCTGGTCGAAGAAGACAAGAAGCACGAAAGACACCCGATC | |
| to SEQ ID | TTCGGAAACATCGTCGACGAAGTCGCATACCACGAAAAGTACCCGACAATCTACCACCTGAGAAAGAAGCTGGTCGACAGCACAGA | |
| NO: 4, Kozak | CAAGGCAGACCTGAGACTGATCTACCTGGCACTGGCACACATGATCAAGTTCAGAGGACACTTCCTGATCGAAGGAGACCTGAACC | |
| sequence, | CGGACAACAGCGACGTCGACAAGCTGTTCATCCAGCTGGTCCAGACATACAACCAGCTGTTCGAAGAAAACCCGATCAACGCAAGC | |
| and 3′ UTR | GGAGTCGACGCAAAGGCAATCCTGAGCGCAAGACTGAGCAAGAGCAGAAGACTGGAAAACCTGATCGCACAGCTGCCGGGAGAAAA | |
| of ALB | GAAGAACGGACTGTTCGGAAACCTGATCGCACTGAGCCTGGGACTGACACCGAACTTCAAGAGCAACTTCGACCTGGCAGAAGACG | |
| | CAAAGCTGCAGCTGAGCAAGGACACATACGACGACGACCTGGACAACCTGCTGGCACAGATCGGAGACCAGTACGCAGACCTGTTC | |
| | CTGGCAGCAAAGAACCTGAGCGACGCAATCCTGCTGAGCGACATCCTGAGAGTCAACACAGAAATCACAAAGGCACCGCTGAGCGC | |
| | AAGCATGATCAAGAGATACGACGAACACCACCAGGACCTGACACTGCTGAAGGCACTGGTCAGACAGCAGCTGCCGGAAAAGTACA | |
| | AGGAAATCTTCTTCGACCAGAGCAAGAACGGATACGCAGGATACATCGACGGAGGAGCAAGCCAGGAAGAATTCTACAAGTTCATC | |
| | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCT | |
| | AGCTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 | GGGTCCCGCAGTCGGCGTCCAGCGGCTCTGCTTGTTCGTGTGTGTGTCGTTGCAGGCCTTATTCGGATCCATGGACAAGAAGTACA | 244 |
| transcript | GCATCGGACTGGACATCGGAACAAACAGCGTCGGATGGGCAGTCATCACAGACGAATACAAGGTCCCGAGCAAGAAGTTCAAGGTC | |
| with 5′ UTR | CTGGGAAACACAGACAGACACAGCATCAAGAAGAACCTGATCGGAGCACTGCTGTTCGACAGCGGAGAAACAGCAGAAGCAACAAG | |
| of HSD, ORF | ACTGAAGAGAACAGCAAGAAGAAGATACACAAGAAGAAAGAACAGAATCTGCTACCTGCAGGAAATCTTCAGCAACGAAATGGCAA | |
| corresponding | AGGTCGACGACAGCTTCTTCCACAGACTGGAAGAAAGCTTCCTGGTCGAAGAAGACAAGAAGCACGAAAGACACCCGATCTTCGGA | |
| to SEQ ID | AACATCGTCGACGAAGTCGCATACCACGAAAAGTACCCGACAATCTACCACCTGAGAAAGAAGCTGGTCGACAGCACAGACAAGGC | |
| NO: 4, and | AGACCTGAGACTGATCTACCTGGCACTGGCACACATGATCAAGTTCAGAGGACACTTCCTGATCGAAGGAGACCTGAACCCGGACA | |
| 3′ UTR of | ACAGCGACGTCGACAAGCTGTTCATCCAGCTGGTCCAGACATACAACCAGCTGTTCGAAGAAAACCCGATCAACGCAAGCGGAGTC | |
| ALB | GACGCAAAGGCAATCCTGAGCGCAAGACTGAGCAAGAGCAGAAGACTGGAAAACCTGATCGCACAGCTGCCGGGAGAAAAGAAGAA | |
| | CGGACTGTTCGGAAACCTGATCGCACTGAGCCTGGGACTGACACCGAACTTCAAGAGCAACTTCGACCTGGCAGAAGACGCAAAGC | |
| | TGCAGCTGAGCAAGGACACATACGACGACGACCTGGACAACCTGCTGGCACAGATCGGAGACCAGTACGCAGACCTGTTCCTGGCA | |
| | GCAAAGAACCTGAGCGACGCAATCCTGCTGAGCGACATCCTGAGAGTCAACACAGAAATCACAAAGGCACCGCTGAGCGCAAGCAT | |
| | GATCAAGAGATACGACGAACACCACCAGGACCTGACACTGCTGAAGGCACTGGTCAGACAGCAGCTGCCGGAAAAGTACAAGGAAA | |
| | TCTTCTTCGACCAGAGCAAGAACGGATACGCAGGATACATCGACGGAGGAGCAAGCCAGGAAGAATTCTACAAGTTCATCAAGCCG | |
| | ATCCTGGAAAAGATGGACGGAAGAGAAGAACTGCTGGTCAAGCTGAACAGAGAAGACCTGCTGAGAAAGCAGAGAACATTCGACAA | |
| | CGGAAGCATCCCGCACCAGATCCACCTGGGAGAACTGCACGCAATCCTGAGAAGACAGGAAGACTTCTACCCGTTCCTGAAGGACA | |
| | ACAGAGAAAAGATCGAAAAGATCCTGACATTCAGAATCCCGTACTACGTCGGACCGCTGGCAAGAGGAAACAGCAGATTCGCATGG | |
| | ATGACAAGAAAGAGCGAAGAAACAATCACACCGTGGAACTTCGAAGAAGTCGTCGACAAGGGAGCAAGCGCACAGAGCTTCATCGA | |
| | AAGAATGACAAACTTCGACAAGAACCTGCCGAACGAAAAGGTCCTGCCGAAGCACAGCCTGCTGTACGAATACTTCACAGTCTACA | |
| | ACGAACTGACAAAGGTCAAGTACGTCACAGAAGGAATGAGAAAGCCGGCATTCCTGAGCGGAGAACAGAAGAAGGCAATCGTCGAC | |
| | CTGCTGTTCAAGACAAACAGAAAGGTCACAGTCAAGCAGCTGAAGGAAGACTACTTCAAGAAGATCGAATGCTTCGACAGCGTCGA | |
| | AATCAGCGGAGTCGAAGACAGATTCAACGCAAGCCTGGGAACATACCACGACCTGCTGAAGATCATCAAGGACAAGGACTTCCTGG | |
| | ACAACGAAGAAAACGAAGACATCCTGGAAGACATCGTCCTGACACTGACACTGTTCGAAGACAGAGAAATGATCGAAGAAAGACTG | |
| | AAGACATACGCACACCTGTTCGACGACAAGGTCATGAAGCAGCTGAAGAGAAGAAGATACACAGGATGGGGAAGACTGAGCAGAAA | |
| | GCTGATCAACGGAATCAGAGACAAGCAGAGCGGAAAGACAATCCTGGACTTCCTGAAGAGCGACGGATTCGCAAACAGAAACTTCA | |
| | TGCAGCTGATCCACGACGACAGCCTGACATTCAAGGAAGACATCCAGAAGGCACAGGTCAGCGGACAGGGAGACAGCCTGCACGAA | |
| | CACATCGCAAACCTGGCAGGAAGCCCGGCAATCAAGAAGGGAATCCTGCAGACAGTCAAGGTCGTCGACGAACTGGTCAAGGTCAT | |
| | GGGAAGACACAAGCCGGAAAACATCGTCATCGAAATGGCAAGAGAAAACCAGACAACACAGAAGGGACAGAAGAACAGCAGAGAAA | |
| | GAATGAAGAGAATCGAAGAAGGAATCAAGGAACTGGGAAGCCAGATCCTGAAGGAACACCCGGTCGAAAACACACAGCTGCAGAAC | |
| | GAAAAGCTGTACCTGTACTACCTGCAGAACGGAAGAGACATGTACGTCGACCAGGAACTGGACATCAACAGACTGAGCGACTACGA | |
| | CGTCGACCACATCGTCCCGCAGAGCTTCCTGAAGGACGACAGCATCGACAACAAGGTCCTGACAAGAAGCGACAAGAACAGAGGAA | |
| | AGAGCGACAACGTCCCGAGCGAAGAAGTCGTCAAGAAGATGAAGAACTACTGGAGACAGCTGCTGAACGCAAAGCTGATCACACAG | |
| | AGAAAGTTCGACAACCTGACAAAGGCAGAGAGAGGAGGACTGAGCGAACTGGACAAGGCAGGATTCATCAAGAGACAGCTGGTCGA | |
| | AACAAGACAGATCACAAAGCACGTCGCACAGATCCTGGACAGCAGAATGAACACAAAGTACGACGAAAACGACAAGCTGATCAGAG | |
| | AAGTCAAGGTCATCACACTGAAGAGCAAGCTGGTCAGCGACTTCAGAAAGGACTTCCAGTTCTACAAGGTCAGAGAAATCAACAAC | |
| | TACCACCACGCACACGACGCATACCTGAACGCAGTCGTCGGAACAGCACTGATCAAGAAGTACCCGAAGCTGGAAAGCGAATTCGT | |
| | CTACGGAGACTACAAGGTCTACGACGTCAGAAAGATGATCGCAAAGAGCGAACAGGAAATCGGAAAGGCAACAGCAAAGTACTTCT | |
| | TCTACAGCAACATCATGAACTTCTTCAAGACAGAAATCACACTGGCAAACGGAGAAATCAGAAAGAGACCGCTGATCGAAACAAAC | |
| | GGAGAAACAGGAGAAATCGTCTGGGACAAGGGAAGAGACTTCGCAACAGTCAGAAAGGTCCTGAGCATGCCGCAGGTCAACATCGT | |
| | CAAGAAGACAGAAGTCCAGACAGGAGGATTCAGCAAGGAAAGCATCCTGCCGAAGAGAAACAGCGACAAGCTGATCGCAAGAAAGA | |
| | AGGACTGGGACCCGAAGAAGTACGGAGGATTCGACAGCCCGACAGTCGCATACAGCGTCCTGGTCGTCGCAAAGGTCGAAAAGGGA | |
| | AAGAGCAAGAAGCTGAAGAGCGTCAAGGAACTGCTGGGAATCACAATCATGGAAAGAAGCAGCTTCGAAAAGAACCCGATCGACTT | |
| | CCTGGAAGCAAAGGGATACAAGGAAGTCAAGAAGGACCTGATCATCAAGCTGCCGAAGTACAGCCTGTTCGAACTGGAAAACGGAA | |
| | GAAAGAGAATGCTGGCAAGCGCAGGAGAACTGCAGAAGGGAAACGAACTGGCACTGCCGAGCAAGTACGTCAACTTCCTGTACCTG | |
| | GCAAGCCACTACGAAAAGCTGAAGGGAAGCCCGGAAGACAACGAACAGAAGCAGCTGTTCGTCGAACAGCACAAGCACTACCTGGA | |
| | CGAAATCATCGAACAGATCAGCGAATTCAGCAAGAGAGTCATCCTGGCAGACGCAAACCTGGACAAGGTCCTGAGCGCATACAACA | |
| | AGCACAGAGACAAGCCGATCAGAGAACAGGCAGAAAACATCATCCACCTGTTCACACTGACAAACCTGGGAGCACCGGCAGCATTC | |
| | AAGTACTTCGACACAACAATCGACAGAAAGAGATACACAAGCACAAAGGAAGTCCTGGACGCAACACTGATCCACCAGAGCATCAC | |
| | AGGACTGTACGAAACAAGAATCGACCTGAGCCAGCTGGGAGGAGACGGAGGAGGAAGCCCGAAGAAGAAGAGAAAGGTCTAGCTAG | |
| | CCATCACATTTAAAAGCATCTCAGCCTACCATGAGAATAAGAGAAAGAAAATGAAGATCAATAGCTTATTCATCTCTTTTTCTTTT | |
| | TCGTTGGTGTAAAGCCAACACCCTGTCTAAAAAACATAAATTTCTTTAATCATTTTGCCTCTTTTCTCTGTGCTTCAATTAATAAA | |
| | AAATGGAAAGAACCTCGAG | |
| | Not Used | 245-249 |
| Cas9 ORF | ATGGACAAGAAGTACAGCATCGGACTGGACATCGGAACAAACAGCGTCGGATGGGCAGTCATCACAGACGAATACAAGGTCCCGAG | 250 |
| with splice | CAAGAAGTTCAAGGTCCTGGGAAACACAGACAGACACAGCATCAAGAAGAACCTGATCGGAGCACTGCTGTTCGACAGCGGAGAAA | |
| junctions | CAGCAGAAGCAACAAGACTGAAGAGAACAGCAAGAAGAAGATACACAAGAAGAAAGAACAGAATCTGCTACCTGCAGGAAATCTTC | |
| removed; | AGCAACGAAATGGCAAAGGTCGACGACAGCTTCTTCCACcggCTGGAAGAAAGCTTCCTGGTCGAAGAAGACAAGAAGCACGAAAG | |
| 12.75% U | ACACCCGATCTTCGGAAACATCGTCGACGAAGTCGCATACCACGAAAAGTACCCGACAATCTACCACCTGAGAAAGAAGCTGGTCG | |
| content | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAaAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCCGAAGAAGAAG | |
| | AGAAAGGTCTAG | |
| Cas9 | GGGTCCCGCAGTCGGCGTCCAGCGGCTCTGCTTGTTCGTGTGTGTGTCGTTGCAGGCCTTATTCGGATCCGCCACCATGGACAAGA | 251 |
| transcript | AGTACAGCATCGGACTGGACATCGGAACAAACAGCGTCGGATGGGCAGTCATCACAGACGAATACAAGGTCCCGAGCAAGAAGTTC | |
| with 5′ UTR | AAGGTCCTGGGAAACACAGACAGACACAGCATCAAGAAGAACCTGATCGGAGCACTGCTGTTCGACAGCGGAGAAACAGCAGAAGC | |
| of HSD, ORF | AACAAGACTGAAGAGAACAGCAAGAAGAAGATACACAAGAAGAAAGAACAGAATCTGCTACCTGCAGGAAATCTTCAGCAACGAAA | |
| corresponding | TGGCAAAGGTCGACGACAGCTTCTTCCACcggCTGGAAGAAAGCTTCCTGGTCGAAGAAGACAAGAAGCACGAAAGACACCCGATC | |
| to SEQ ID | TTCGGAAACATCGTCGACGAAGTCGCATACCACGAAAAGTACCCGACAATCTACCACCTGAGAAAGAAGCTGGTCGACAGCACAGA | |
| NO: 50, | CAAGGCAGACCTGAGACTGATCTACCTGGCACTGGCACACATGATCAAGTTCAGAGGACACTTCCTGATCGAAGGAGACCTGAACC | |
| Kozak | CGGACAACAGCGACGTCGACAAGCTGTTCATCCAGCTGGTCCAGACATACAACCAGCTGTTCGAAGAAAACCCGATCAACGCAAGC | |
| sequence, | GGAGTCGACGCAAAGGCAATCCTGAGCGCAAGACTGAGCAAGAGCAGAAGACTGGAAAACCTGATCGCACAGCTGCCGGGAGAAAA | |
| and 3′ UTR | GAAGAACGGACTGTTCGGAAACCTGATCGCACTGAGCCTGGGACTGACACCGAACTTCAAGAGCAACTTCGACCTGGCAGAAGACG | |
| of ALB | CAAAGCTGCAGCTGAGCAAGGACACATACGACGACGACCTGGACAACCTGCTGGCACAGATCGGAGACCAGTACGCAGACCTGTTC | |
| | CTGGCAGCAAAGAACCTGAGCGACGCAATCCTGCTGAGCGACATCCTGAGAGTCAACACAGAAATCACAAAGGCACCGCTGAGCGC | |
| | AAGCATGATCAAGAGATACGACGAACACCACCAGGACCTGACACTGCTGAAGGCACTGGTCAGACAGCAGCTGCCGGAAAAGTACA | |
| | AGGAAATCTTCTTCGACCAGAGCAAGAACGGATACGCAGGATACATCGACGGAGGAGCAAGCCAGGAAGAATTCTACAAGTTCATC | |
| | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAaAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCT | |
| | AGCTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 ORF | ATGGACAAGAAGTACAGCATCGGCCTGGACATCGGCACCAACAGCGTGGGCTGGGCCGTGATCACCGACGAGTACAAGGTGCCCAG | 252 |
| with minimal | CAAGAAGTTCAAGGTGCTGGGCAACACCGACAGACACAGCATCAAGAAGAACCTGATCGGCGCCCTGCTGTTCGACAGCGGCGAGA | |
| uridine | CCGCCGAGGCCACCAGACTGAAGAGAACCGCCAGAAGAAGATACACCAGAAGAAAGAACAGAATCTGCTACCTGCAGGAGATCTTC | |
| codons | AGCAACGAGATGGCCAAGGTGGACGACAGCTTCTTCCACAGACTGGAGGAGAGCTTCCTGGTGGAGGAGGACAAGAAGCACGAGAG | |
| frequently | ACACCCCATCTTCGGCAACATCGTGGACGAGGTGGCCTACCACGAGAAGTACCCCACCATCTACCACCTGAGAAAGAAGCTGGTGG | |
| used in | ACAGCACCGACAAGGCCGACCTGAGACTGATCTACCTGGCCCTGGCCCACATGATCAAGTTCAGAGGCCACTTCCTGATCGAGGGC | |
| humans in | GACCTGAACCCCGACAACAGCGACGTGGACAAGCTGTTCATCCAGCTGGTGCAGACCTACAACCAGCTGTTCGAGGAGAACCCCAT | |
| general; | CAACGCCAGCGGCGTGGACGCCAAGGCCATCCTGAGCGCCAGACTGAGCAAGAGCAGAAGACTGGAGAACCTGATCGCCCAGCTGC | |
| 12.75% U | CCGGCGAGAAGAAGAACGGCCTGTTCGGCAACCTGATCGCCCTGAGCCTGGGCCTGACCCCCAACTTCAAGAGCAACTTCGACCTG | |
| content | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGAGAGTGAACACCGAGATCACCAAGGCCC | |
| | CCCTGAGCGCCAGCATGATCAAGAGATACGACGAGCACCACCAGGACCTGACCCTGCTGAAGGCCCTGGTGAGACAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACAGAGAGGACCTGCTGAGAAAGC | |
| | AGAGAACCTTCGACAACGGCAGCATCCCCCACCAGATCCACCTGGGCGAGCTGCACGCCATCCTGAGAAGACAGGAGGACTTCTAC | |
| | CCCTTCCTGAAGGACAACAGAGAGAAGATCGAGAAGATCCTGACCTTCAGAATCCCCTACTACGTGGGCCCCCTGGCCAGAGGCAA | |
| | CAGCAGATTCGCCTGGATGACCAGAAAGAGCGAGGAGACCATCACCCCCTGGAACTTCGAGGAGGTGGTGGACAAGGGCGCCAGCG | |
| | CCCAGAGCTTCATCGAGAGAATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGAGAAAGCCCGCCTTCCTGAGCGGCGAGCAGAA | |
| | GAAGGCCATCGTGGACCTGCTGTTCAAGACCAACAGAAAGGTGACCGTGAAGCAGCTGAAGGAGGACTACTTCAAGAAGATCGAGT | |
| | GCTTCGACAGCGTGGAGATCAGCGGCGTGGAGGACAGATTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACAGAGAGAT | |
| | GATCGAGGAGAGACTGAAGACCTACGCCCACCTGTTCGACGACAAGGTGATGAAGCAGCTGAAGAGAAGAAGATACACCGGCTGGG | |
| | GCAGACTGAGCAGAAAGCTGATCAACGGCATCAGAGACAAGCAGAGCGGCAAGACCATCCTGGACTTCCTGAAGAGCGACGGCTTC | |
| | GCCAACAGAAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCAGACACAAGCCCGAGAACATCGTGATCGAGATGGCCAGAGAGAACCAGACCACCCAGAAGGGCCAG | |
| | AAGAACAGCAGAGAGAGAATGAAGAGAATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCAGAGACATGTACGTGGACCAGGAGCTGGACATCAACA | |
| | GACTGAGCGACTACGACGTGGACCACATCGTGCCCCAGAGCTTCCTGAAGGACGACAGCATCGACAACAAGGTGCTGACCAGAAGC | |
| | GACAAGAACAGAGGCAAGAGCGACAACGTGCCCAGCGAGGAGGTGGTGAAGAAGATGAAGAACTACTGGAGACAGCTGCTGAACGC | |
| | CAAGCTGATCACCCAGAGAAAGTTCGACAACCTGACCAAGGCCGAGAGAGGCGGCCTGAGCGAGCTGGACAAGGCCGGCTTCATCA | |
| | AGAGACAGCTGGTGGAGACCAGACAGATCACCAAGCACGTGGCCCAGATCCTGGACAGCAGAATGAACACCAAGTACGACGAGAAC | |
| | GACAAGCTGATCAGAGAGGTGAAGGTGATCACCCTGAAGAGCAAGCTGGTGAGCGACTTCAGAAAGGACTTCCAGTTCTACAAGGT | |
| | GAGAGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGAGAAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCAGAAAGAGACC | |
| | CCTGATCGAGACCAACGGCGAGACCGGCGAGATCGTGTGGGACAAGGGCAGAGACTTCGCCACCGTGAGAAAGGTGCTGAGCATGC | |
| | CCCAGGTGAACATCGTGAAGAAGACCGAGGTGCAGACCGGCGGCTTCAGCAAGGAGAGCATCCTGCCCAAGAGAAACAGCGACAAG | |
| | CTGATCGCCAGAAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGAGAAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCAGAAAGAGAATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGAGAGTGATCCTGGCCGACGCCAACCTGGACAAGGTG | |
| | CTGAGCGCCTACAACAAGCACAGAGACAAGCCCATCAGAGAGCAGGCCGAGAACATCATCCACCTGTTCACCCTGACCAACCTGGG | |
| | CGCCCCCGCCGCCTTCAAGTACTTCGACACCACCATCGACAGAAAGAGATACACCAGCACCAAGGAGGTGCTGGACGCCACCCTGA | |
| | TCCACCAGAGCATCACCGGCCTGTACGAGACCAGAATCGACCTGAGCCAGCTGGGCGGCGACGGCGGCGGCAGCCCCAAGAAGAAG | |
| | AGAAAGGTGTGA | |
| Cas9 | GGGTCCCGCAGTCGGCGTCCAGCGGCTCTGCTTGTTCGTGTGTGTGTCGTTGCAGGCCTTATTCGGATCCGCCACCATGGACAAGA | 253 |
| transcript | AGTACAGCATCGGCCTGGACATCGGCACCAACAGCGTGGGCTGGGCCGTGATCACCGACGAGTACAAGGTGCCCAGCAAGAAGTTC | |
| with 5′ UTR | AAGGTGCTGGGCAACACCGACAGACACAGCATCAAGAAGAACCTGATCGGCGCCCTGCTGTTCGACAGCGGCGAGACCGCCGAGGC | |
| of HSD, ORF | CACCAGACTGAAGAGAACCGCCAGAAGAAGATACACCAGAAGAAAGAACAGAATCTGCTACCTGCAGGAGATCTTCAGCAACGAGA | |
| corresponding | TGGCCAAGGTGGACGACAGCTTCTTCCACAGACTGGAGGAGAGCTTCCTGGTGGAGGAGGACAAGAAGCACGAGAGACACCCCATC | |
| to SEQ ID | TTCGGCAACATCGTGGACGAGGTGGCCTACCACGAGAAGTACCCCACCATCTACCACCTGAGAAAGAAGCTGGTGGACAGCACCGA | |
| NO: 52, | CAAGGCCGACCTGAGACTGATCTACCTGGCCCTGGCCCACATGATCAAGTTCAGAGGCCACTTCCTGATCGAGGGCGACCTGAACC | |
| Kozak | CCGACAACAGCGACGTGGACAAGCTGTTCATCCAGCTGGTGCAGACCTACAACCAGCTGTTCGAGGAGAACCCCATCAACGCCAGC | |
| sequence, | GGCGTGGACGCCAAGGCCATCCTGAGCGCCAGACTGAGCAAGAGCAGAAGACTGGAGAACCTGATCGCCCAGCTGCCCGGCGAGAA | |
| and 3′ UTR | GAAGAACGGCCTGTTCGGCAACCTGATCGCCCTGAGCCTGGGCCTGACCCCCAACTTCAAGAGCAACTTCGACCTGGCCGAGGACG | |
| of ALB | CCAAGCTGCAGCTGAGCAAGGACACCTACGACGACGACCTGGACAACCTGCTGGCCCAGATCGGCGACCAGTACGCCGACCTGTTC | |
| | CTGGCCGCCAAGAACCTGAGCGACGCCATCCTGCTGAGCGACATCCTGAGAGTGAACACCGAGATCACCAAGGCCCCCCTGAGCGC | |
| | CAGCATGATCAAGAGATACGACGAGCACCACCAGGACCTGACCCTGCTGAAGGCCCTGGTGAGACAGCAGCTGCCCGAGAAGTACA | |
| | AGGAGATCTTCTTCGACCAGAGCAAGAACGGCTACGCCGGCTACATCGACGGCGGCGCCAGCCAGGAGGAGTTCTACAAGTTCATC | |
| | AAGCCCATCCTGGAGAAGATGGACGGCACCGAGGAGCTGCTGGTGAAGCTGAACAGAGAGGACCTGCTGAGAAAGCAGAGAACCTT | |
| | CGACAACGGCAGCATCCCCCACCAGATCCACCTGGGCGAGCTGCACGCCATCCTGAGAAGACAGGAGGACTTCTACCCCTTCCTGA | |
| | AGGACAACAGAGAGAAGATCGAGAAGATCCTGACCTTCAGAATCCCCTACTACGTGGGCCCCCTGGCCAGAGGCAACAGCAGATTC | |
| | GCCTGGATGACCAGAAAGAGCGAGGAGACCATCACCCCCTGGAACTTCGAGGAGGTGGTGGACAAGGGCGCCAGCGCCCAGAGCTT | |
| | CATCGAGAGAATGACCAACTTCGACAAGAACCTGCCCAACGAGAAGGTGCTGCCCAAGCACAGCCTGCTGTACGAGTACTTCACCG | |
| | TGTACAACGAGCTGACCAAGGTGAAGTACGTGACCGAGGGCATGAGAAAGCCCGCCTTCCTGAGCGGCGAGCAGAAGAAGGCCATC | |
| | GTGGACCTGCTGTTCAAGACCAACAGAAAGGTGACCGTGAAGCAGCTGAAGGAGGACTACTTCAAGAAGATCGAGTGCTTCGACAG | |
| | CGTGGAGATCAGCGGCGTGGAGGACAGATTCAACGCCAGCCTGGGCACCTACCACGACCTGCTGAAGATCATCAAGGACAAGGACT | |
| | TCCTGGACAACGAGGAGAACGAGGACATCCTGGAGGACATCGTGCTGACCCTGACCCTGTTCGAGGACAGAGAGATGATCGAGGAG | |
| | AGACTGAAGACCTACGCCCACCTGTTCGACGACAAGGTGATGAAGCAGCTGAAGAGAAGAAGATACACCGGCTGGGGCAGACTGAG | |
| | CAGAAAGCTGATCAACGGCATCAGAGACAAGCAGAGCGGCAAGACCATCCTGGACTTCCTGAAGAGCGACGGCTTCGCCAACAGAA | |
| | ACTTCATGCAGCTGATCCACGACGACAGCCTGACCTTCAAGGAGGACATCCAGAAGGCCCAGGTGAGCGGCCAGGGCGACAGCCTG | |
| | CACGAGCACATCGCCAACCTGGCCGGCAGCCCCGCCATCAAGAAGGGCATCCTGCAGACCGTGAAGGTGGTGGACGAGCTGGTGAA | |
| | GGTGATGGGCAGACACAAGCCCGAGAACATCGTGATCGAGATGGCCAGAGAGAACCAGACCACCCAGAAGGGCCAGAAGAACAGCA | |
| | GAGAGAGAATGAAGAGAATCGAGGAGGGCATCAAGGAGCTGGGCAGCCAGATCCTGAAGGAGCACCCCGTGGAGAACACCCAGCTG | |
| | CAGAACGAGAAGCTGTACCTGTACTACCTGCAGAACGGCAGAGACATGTACGTGGACCAGGAGCTGGACATCAACAGACTGAGCGA | |
| | CTACGACGTGGACCACATCGTGCCCCAGAGCTTCCTGAAGGACGACAGCATCGACAACAAGGTGCTGACCAGAAGCGACAAGAACA | |
| | GAGGCAAGAGCGACAACGTGCCCAGCGAGGAGGTGGTGAAGAAGATGAAGAACTACTGGAGACAGCTGCTGAACGCCAAGCTGATC | |
| | ACCCAGAGAAAGTTCGACAACCTGACCAAGGCCGAGAGAGGCGGCCTGAGCGAGCTGGACAAGGCCGGCTTCATCAAGAGACAGCT | |
| | GGTGGAGACCAGACAGATCACCAAGCACGTGGCCCAGATCCTGGACAGCAGAATGAACACCAAGTACGACGAGAACGACAAGCTGA | |
| | TCAGAGAGGTGAAGGTGATCACCCTGAAGAGCAAGCTGGTGAGCGACTTCAGAAAGGACTTCCAGTTCTACAAGGTGAGAGAGATC | |
| | AACAACTACCACCACGCCCACGACGCCTACCTGAACGCCGTGGTGGGCACCGCCCTGATCAAGAAGTACCCCAAGCTGGAGAGCGA | |
| | GTTCGTGTACGGCGACTACAAGGTGTACGACGTGAGAAAGATGATCGCCAAGAGCGAGCAGGAGATCGGCAAGGCCACCGCCAAGT | |
| | ACTTCTTCTACAGCAACATCATGAACTTCTTCAAGACCGAGATCACCCTGGCCAACGGCGAGATCAGAAAGAGACCCCTGATCGAG | |
| | ACCAACGGCGAGACCGGCGAGATCGTGTGGGACAAGGGCAGAGACTTCGCCACCGTGAGAAAGGTGCTGAGCATGCCCCAGGTGAA | |
| | CATCGTGAAGAAGACCGAGGTGCAGACCGGCGGCTTCAGCAAGGAGAGCATCCTGCCCAAGAGAAACAGCGACAAGCTGATCGCCA | |
| | GAAAGAAGGACTGGGACCCCAAGAAGTACGGCGGCTTCGACAGCCCCACCGTGGCCTACAGCGTGCTGGTGGTGGCCAAGGTGGAG | |
| | AAGGGCAAGAGCAAGAAGCTGAAGAGCGTGAAGGAGCTGCTGGGCATCACCATCATGGAGAGAAGCAGCTTCGAGAAGAACCCCAT | |
| | CGACTTCCTGGAGGCCAAGGGCTACAAGGAGGTGAAGAAGGACCTGATCATCAAGCTGCCCAAGTACAGCCTGTTCGAGCTGGAGA | |
| | ACGGCAGAAAGAGAATGCTGGCCAGCGCCGGCGAGCTGCAGAAGGGCAACGAGCTGGCCCTGCCCAGCAAGTACGTGAACTTCCTG | |
| | TACCTGGCCAGCCACTACGAGAAGCTGAAGGGCAGCCCCGAGGACAACGAGCAGAAGCAGCTGTTCGTGGAGCAGCACAAGCACTA | |
| | CCTGGACGAGATCATCGAGCAGATCAGCGAGTTCAGCAAGAGAGTGATCCTGGCCGACGCCAACCTGGACAAGGTGCTGAGCGCCT | |
| | ACAACAAGCACAGAGACAAGCCCATCAGAGAGCAGGCCGAGAACATCATCCACCTGTTCACCCTGACCAACCTGGGCGCCCCCGCC | |
| | GCCTTCAAGTACTTCGACACCACCATCGACAGAAAGAGATACACCAGCACCAAGGAGGTGCTGGACGCCACCCTGATCCACCAGAG | |
| | CATCACCGGCCTGTACGAGACCAGAATCGACCTGAGCCAGCTGGGCGGCGACGGCGGCGGCAGCCCCAAGAAGAAGAGAAAGGTGT | |
| | GACTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 ORF | ATGGACAAAAAATACAGCATAGGGCTAGACATAGGGACGAACAGCGTAGGGTGGGCGGTAATAACGGACGAATACAAAGTACCGAG | 254 |
| with minimal | CAAAAAATTCAAAGTACTAGGGAACACGGACCGACACAGCATAAAAAAAAACCTAATAGGGGCGCTACTATTCGACAGCGGGGAAA | |
| uridine | CGGCGGAAGCGACGCGACTAAAACGAACGGCGCGACGACGATACACGCGACGAAAAAACCGAATATGCTACCTACAAGAAATATTC | |
| codons | AGCAACGAAATGGCGAAAGTAGACGACAGCTTCTTCCACCGACTAGAAGAAAGCTTCCTAGTAGAAGAAGACAAAAAACACGAACG | |
| infrequently | ACACCCGATATTCGGGAACATAGTAGACGAAGTAGCGTACCACGAAAAATACCCGACGATATACCACCTACGAAAAAAACTAGTAG | |
| used in | ACAGCACGGACAAAGCGGACCTACGACTAATATACCTAGCGCTAGCGCACATGATAAAATTCCGAGGGCACTTCCTAATAGAAGGG | |
| humans in | GACCTAAACCCGGACAACAGCGACGTAGACAAACTATTCATACAACTAGTACAAACGTACAACCAACTATTCGAAGAAAACCCGAT | |
| general; | AAACGCGAGCGGGGTAGACGCGAAAGCGATACTAAGCGCGCGACTAAGCAAAAGCCGACGACTAGAAAACCTAATAGCGCAACTAC | |
| 12.75% U | CGGGGGAAAAAAAAAACGGGCTATTCGGGAACCTAATAGCGCTAAGCCTAGGGCTAACGCCGAACTTCAAAAGCAACTTCGACCTA | |
| content | GCGGAAGACGCGAAACTACAACTAAGCAAAGACACGTACGACGACGACCTAGACAACCTACTAGCGCAAATAGGGGACCAATACGC | |
| | GGACCTATTCCTAGCGGCGAAAAACCTAAGCGACGCGATACTACTAAGCGACATACTACGAGTAAACACGGAAATAACGAAAGCGC | |
| | CGCTAAGCGCGAGCATGATAAAACGATACGACGAACACCACCAAGACCTAACGCTACTAAAAGCGCTAGTACGACAACAACTACCG | |
| | GAAAAATACAAAGAAATATTCTTCGACCAAAGCAAAAACGGGTACGCGGGGTACATAGACGGGGGGGCGAGCCAAGAAGAATTCTA | |
| | CAAATTCATAAAACCGATACTAGAAAAAATGGACGGGACGGAAGAACTACTAGTAAAACTAAACCGAGAAGACCTACTACGAAAAC | |
| | AACGAACGTTCGACAACGGGAGCATACCGCACCAAATACACCTAGGGGAACTACACGCGATACTACGACGACAAGAAGACTTCTAC | |
| | CCGTTCCTAAAAGACAACCGAGAAAAAATAGAAAAAATACTAACGTTCCGAATACCGTACTACGTAGGGCCGCTAGCGCGAGGGAA | |
| | CAGCCGATTCGCGTGGATGACGCGAAAAAGCGAAGAAACGATAACGCCGTGGAACTTCGAAGAAGTAGTAGACAAAGGGGCGAGCG | |
| | CGCAAAGCTTCATAGAACGAATGACGAACTTCGACAAAAACCTACCGAACGAAAAAGTACTACCGAAACACAGCCTACTATACGAA | |
| | TACTTCACGGTATACAACGAACTAACGAAAGTAAAATACGTAACGGAAGGGATGCGAAAACCGGCGTTCCTAAGCGGGGAACAAAA | |
| | AAAAGCGATAGTAGACCTACTATTCAAAACGAACCGAAAAGTAACGGTAAAACAACTAAAAGAAGACTACTTCAAAAAAATAGAAT | |
| | GCTTCGACAGCGTAGAAATAAGCGGGGTAGAAGACCGATTCAACGCGAGCCTAGGGACGTACCACGACCTACTAAAAATAATAAAA | |
| | GACAAAGACTTCCTAGACAACGAAGAAAACGAAGACATACTAGAAGACATAGTACTAACGCTAACGCTATTCGAAGACCGAGAAAT | |
| | GATAGAAGAACGACTAAAAACGTACGCGCACCTATTCGACGACAAAGTAATGAAACAACTAAAACGACGACGATACACGGGGTGGG | |
| | GGCGACTAAGCCGAAAACTAATAAACGGGATACGAGACAAACAAAGCGGGAAAACGATACTAGACTTCCTAAAAAGCGACGGGTTC | |
| | GCGAACCGAAACTTCATGCAACTAATACACGACGACAGCCTAACGTTCAAAGAAGACATACAAAAAGCGCAAGTAAGCGGGCAAGG | |
| | GGACAGCCTACACGAACACATAGCGAACCTAGCGGGGAGCCCGGCGATAAAAAAAGGGATACTACAAACGGTAAAAGTAGTAGACG | |
| | AACTAGTAAAAGTAATGGGGCGACACAAACCGGAAAAGATAGTAATAGAAATGGCGCGAGAAAACCAAACGACGCAAAAAGGGCAA | |
| | AAAAACAGCCGAGAACGAATGAAACGIATAGAAGAAGGGATAAAAGAACTAGGGAGCCAAATACTAAAAGAACACCCGGTAGAAAA | |
| | CACGCAACTACAAAACGAAAAACTATACCTATACTACCTACAAAACGGGCGAGACATGTACGTAGACCAAGAACTAGACATAAACC | |
| | GACTAAGCGACTACGACGTAGACCACATAGTACCGCAAAGCTTCCTAAAAGACGACAGCATAGACAACAAAGTACTAACGCGAAGC | |
| | GACAAAAACCGAGGGAAAAGCGACAACGTACCGAGCGAAGAAGTAGTAAAAAAAATGAAAAACTACTGGCGACAACTACTAAACGC | |
| | GAAACTAATAACGCAACGAAAATTCGACAACCTAACGAAAGCGGAACGAGGGGGGCTAAGCGAACTAGACAAAGCGGGGTTCATAA | |
| | AACGACAACTAGTAGAAACGCGACAAATAACGAAACACGTAGCGCAAATACTAGACAGCCGAATGAACACGAAATACGACGAAAAC | |
| | GACAAACTAATACGAGAAGTAAAAGTAATAACGCTAAAAAGCAAACTAGTAAGCGACTTCCGAAAAGACTTCCAATTCTACAAAGT | |
| | ACGAGAAATAAACAACTACCACCACGCGCACGACGCGTACCTAAACGCGGTAGTAGGGACGGCGCTAATAAAAAAATACCCGAAAC | |
| | TAGAAAGCGAATTCGTATACGGGGACTACAAAGTATACGACGTACGAAAAATGATAGCGAAAAGCGAACAAGAAATAGGGAAAGCG | |
| | ACGGCGAAATACTTCTTCTACAGCAACATAATGAACTTCTTCAAAACGGAAATAACGCTAGCGAACGGGGAAATACGAAAACGACC | |
| | GCTAATAGAAACGAACGGGGAAACGGGGGAAATAGTATGGGACAAAGGGCGAGACTTCGCGACGGTACGAAAAGTACTAAGCATGC | |
| | CGCAAGTAAACATAGTAAAAAAAACGGAAGTACAAACGGGGGGGTTCAGCAAAGAAAGCATACTACCGAAACGAAACAGCGACAAA | |
| | CTAATAGCGCGAAAAAAAGACTGGGACCCGAAAAAATACGGGGGGTTCGACAGCCCGACGGTAGCGTACAGCGTACTAGTAGTAGC | |
| | GAAAGTAGAAAAAGGGAAAAGCAAAAAACTAAAAAGCGTAAAAGAACTACTAGGGATAACGATAATGGAACGAAGCAGCTTCGAAA | |
| | AAAACCCGATAGACTTCCTAGAAGCGAAAGGGTACAAAGAAGTAAAAAAAGACCTAATAATAAAACTACCGAAATACAGCCTATTC | |
| | GAACTAGAAAACGGGCGAAAACGAATGCTAGCGAGCGCGGGGGAACTACAAAAAGGGAACGAACTAGCGCTACCGAGCAAATACGT | |
| | AAACTTCCTATACCTAGCGAGCCACTACGAAAAACTAAAAGGGAGCCCGGAAGACAACGAACAAAAACAACTATTCGTAGAACAAC | |
| | ACAAACACTACCTAGACGAAATAATAGAACAAATAAGCGAATTCAGCAAACGAGTAATACTAGCGGACGCGAACCTAGACAAAGTA | |
| | CTAAGCGCGTACAACAAACACCGAGACAAACCGATACGAGAACAAGCGGAAAAGATAATACACCTATTCACGCTAACGAACCTAGG | |
| | GGCGCCGGCGGCGTTCAAATACTTCGACACGACGATAGACCGAAAACGATACACGAGCACGAAAGAAGTACTAGACGCGACGCTAA | |
| | TACACCAAAGCATAACGGGGCTATACGAAACGCGAATAGACCTAAGCCAACTAGGGGGGGACGGGGGGGGGAGCCCGAAAAAAAAA | |
| | CGAAAAGTATGA | |
| Cas9 | GGGTCCCGCAGTCGGCGTCCAGCGGCTCTGCTTGTTCGTGTGTGTGTCGTTGCAGGCCTTATTCGGATCCGCCACCATGGACAAAA | 255 |
| transcript | AATACAGCATAGGGCTAGACATAGGGACGAACAGCGTAGGGTGGGCGGTAATAACGGACGAATACAAAGTACCGAGCAAAAAATTC | |
| with 5′ UTR | AAAGTACTAGGGAACACGGACCGACACAGCATAAAAAAAAACCTAATAGGGGCGCTACTATTCGACAGCGGGGAAACGGCGGAAGC | |
| of HSD, ORF | GACGCGACTAAAACGAACGGCGCGACGACGATACACGCGACGAAAAAACCGAATATGCTACCTACAAGAAATATTCAGCAACGAAA | |
| corresponding | TGGCGAAAGTAGACGACAGCTTCTTCCACCGACTAGAAGAAAGCTTCCTAGTAGAAGAAGACAAAAAACACGAACGACACCCGATA | |
| to SEQ ID | TTCGGGAACATAGTAGACGAAGTAGCGTACCACGAAAAATACCCGACGATATACCACCTACGAAAAAAACTAGTAGACAGCACGGA | |
| NO: 54, | CAAAGCGGACCTACGACTAATATACCTAGCGCTAGCGCACATGATAAAATTCCGAGGGCACTTCCTAATAGAAGGGGACCTAAACC | |
| Kozak | CGGACAACAGCGACGTAGACAAACTATTCATACAACTAGTACAAACGTACAACCAACTATTCGAAGAAAACCCGATAAACGCGAGC | |
| sequence, | GGGGTAGACGCGAAAGCGATACTAAGCGCGCGACTAAGCAAAAGCCGACGACTAGAAAACCTAATAGCGCAACTACCGGGGGAAAA | |
| and 3′ UTR | AAAAAACGGGCTATTCGGGAACCTAATAGCGCTAAGCCTAGGGCTAACGCCGAACTTCAAAAGCAACTTCGACCTAGCGGAAGACG | |
| of ALB | CGAAACTACAACTAAGCAAAGACACGTACGACGACGACCTAGACAACCTACTAGCGCAAATAGGGGACCAATACGCGGACCTATTC | |
| | CTAGCGGCGAAAAACCTAAGCGACGCGATACTACTAAGCGACATACTACGAGTAAACACGGAAATAACGAAAGCGCCGCTAAGCGC | |
| | GAGCATGATAAAACGATACGACGAACACCACCAAGACCTAACGCTACTAAAAGCGCTAGTACGACAACAACTACCGGAAAAATACA | |
| | AAGAAATATTCTTCGACCAAAGCAAAAACGGGTACGCGGGGTACATAGACGGGGGGGCGAGCCAAGAAGAATTCTACAAATTCATA | |
| | AAACCGATACTAGAAAAAATGGACGGGACGGAAGAACTACTAGTAAAACTAAACCGAGAAGACCTACTACGAAAACAACGAACGTT | |
| | CGACAACGGGAGCATACCGCACCAAATACACCTAGGGGAACTACACGCGATACTACGACGACAAGAAGACTTCTACCCGTTCCTAA | |
| | AAGACAACCGAGAAAAAATAGAAAAAATACTAACGTTCCGAATACCGTACTACGTAGGGCCGCTAGCGCGAGGGAACAGCCGATTC | |
| | GCGTGGATGACGCGAAAAAGCGAAGAAACGATAACGCCGTGGAACTTCGAAGAAGTAGTAGACAAAGGGGCGAGCGCGCAAAGCTT | |
| | CATAGAACGAATGACGAACTTCGACAAAAACCTACCGAACGAAAAAGTACTACCGAAACACAGCCTACTATACGAATACTTCACGG | |
| | TATACAACGAACTAACGAAAGTAAAATACGTAACGGAAGGGATGCGAAAACCGGCGTTCCTAAGCGGGGAACAAAAAAAAGCGATA | |
| | GTAGACCTACTATTCAAAACGAACCGAAAAGTAACGGTAAAACAACTAAAAGAAGACTACTTCAAAAAAATAGAATGCTTCGACAG | |
| | CGTAGAAATAAGCGGGGTAGAAGACCGATTCAACGCGAGCCTAGGGACGTACCACGACCTACTAAAAATAATAAAAGACAAAGACT | |
| | TCCTAGACAACGAAGAAAACGAAGACATACTAGAAGACATAGTACTAACGCTAACGCTATTCGAAGACCGAGAAATGATAGAAGAA | |
| | CGACTAAAAACGTACGCGCACCTATTCGACGACAAAGTAATGAAACAACTAAAACGACGACGATACACGGGGTGGGGGCGACTAAG | |
| | CCGAAAACTAATAAACGGGATACGAGACAAACAAAGCGGGAAAACGATACTAGACTTCCTAAAAAGCGACGGGTTCGCGAACCGAA | |
| | ACTTCATGCAACTAATACACGACGACAGCCTAACGTTCAAAGAAGACATACAAAAAGCGCAAGTAAGCGGGCAAGGGGACAGCCTA | |
| | CACGAACACATAGCGAACCTAGCGGGGAGCCCGGCGATAAAAAAAGGGATACTACAAACGGTAAAAGTAGTAGACGAACTAGTAAA | |
| | AGTAATGGGGCGACACAAACCGGAAAACATAGTAATAGAAATGGCGCGAGAAAACCAAACGACGCAAAAAGGGCAAAAAAACAGCC | |
| | GAGAACGAATGAAACGAATAGAAGAAGGGATAAAAGAACTAGGGAGCCAAATACTAAAAGAACACCCGGTAGAAAACACGCAACTA | |
| | CAAAACGAAAAACTATACCTATACTACCTACAAAACGGGCGAGACATGTACGTAGACCAAGAACTAGACATAAACCGACTAAGCGA | |
| | CTACGACGTAGACCACATAGTACCGCAAAGCTTCCTAAAAGACGACAGCATAGACAACAAAGTACTAACGCGAAGCGACAAAAACC | |
| | GAGGGAAAAGCGACAACGTACCGAGCGAAGAAGTAGTAAAAAAAATGAAAAACTACTGGCGACAACTACTAAACGCGAAACTAATA | |
| | ACGCAACGAAAATTCGACAACCTAACGAAAGCGGAACGAGGGGGGCTAAGCGAACTAGACAAAGCGGGGTTCATAAAACGACAACT | |
| | AGTAGAAACGCGACAAATAACGAAACACGTAGCGCAAATACTAGACAGCCGAATGAACACGAAATACGACGAAAACGACAAACTAA | |
| | TACGAGAAGTAAAAGTAATAACGCTAAAAAGCAAACTAGTAAGCGACTTCCGAAAAGACTTCCAATTCTACAAAGTACGAGAAATA | |
| | AACAACTACCACCACGCGCACGACGCGTACCTAAACGCGGTAGTAGGGACGGCGCTAATAAAAAAATACCCGAAACTAGAAAGCGA | |
| | ATTCGTATACGGGGACTACAAAGTATACGACGTACGAAAAATGATAGCGAAAAGCGAACAAGAAATAGGGAAAGCGACGGCGAAAT | |
| | ACTTCTTCTACAGCAACATAATGAACTTCTTCAAAACGGAAATAACGCTAGCGAACGGGGAAATACGAAAACGACCGCTAATAGAA | |
| | ACGAACGGGGAAACGGGGGAAATAGTATGGGACAAAGGGCGAGACTTCGCGACGGTACGAAAAGTACTAAGCATGCCGCAAGTAAA | |
| | CATAGTAAAAAAAACGGAAGTACAAACGGGGGGGTTCAGCAAAGAAAGCATACTACCGAAACGAAACAGCGACAAACTAATAGCGC | |
| | GAAAAAAAGACTGGGACCCGAAAAAATACGGGGGGTTCGACAGCCCGACGGTAGCGTACAGCGTACTAGTAGTAGCGAAAGTAGAA | |
| | AAAGGGAAAAGCAAAAAACTAAAAAGCGTAAAAGAACTACTAGGGATAACGATAATGGAACGAAGCAGCTTCGAAAAAAACCCGAT | |
| | AGACTTCCTAGAAGCGAAAGGGTACAAAGAAGTAAAAAAAGACCTAATAATAAAACTACCGAAATACAGCCTATTCGAACTAGAAA | |
| | ACGGGCGAAAACGAATGCTAGCGAGCGCGGGGGAACTACAAAAAGGGAACGAACTAGCGCTACCGAGCAAATACGTAAACTTCCTA | |
| | TACCTAGCGAGCCACTACGAAAAACTAAAAGGGAGCCCGGAAGACAACGAACAAAAACAACTATTCGTAGAACAACACAAACACTA | |
| | CCTAGACGAAATAATAGAACAAATAAGCGAATTCAGCAAACGAGTAATACTAGCGGACGCGAACCTAGACAAAGTACTAAGCGCGT | |
| | ACAACAAACACCGAGACAAACCGATACGAGAACAAGCGGAAAACATAATACACCTATTCACGCTAACGAACCTAGGGGCGCCGGCG | |
| | GCGTTCAAATACTTCGACACGACGATAGACCGAAAACGATACACGAGCACGAAAGAAGTACTAGACGCGACGCTAATACACCAAAG | |
| | CATAACGGGGCTATACGAAACGCGAATAGACCTAAGCCAACTAGGGGGGGACGGGGGGGGGAGCCCGAAAAAAAAACGAAAAGTAT | |
| | GACTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 | AGGTCCCGCAGTCGGCGTCCAGCGGCTCTGCTTGTTCGTGTGTGTGTCGTTGCAGGCCTTATTCGGATCCGCCACCATGGACAAGA | 256 |
| transcript | AGTACAGCATCGGACTGGACATCGGAACAAACAGCGTCGGATGGGCAGTCATCACAGACGAATACAAGGTCCCGAGCAAGAAGTTC | |
| with AGG as | AAGGTCCTGGGAAACACAGACAGACACAGCATCAAGAAGAACCTGATCGGAGCACTGCTGTTCGACAGCGGAGAAACAGCAGAAGC | |
| first three | AACAAGACTGAAGAGAACAGCAAGAAGAAGATACACAAGAAGAAAGAACAGAATCTGCTACCTGCAGGAAATCTTCAGCAACGAAA | |
| nucleotides | TGGCAAAGGTCGACGACAGCTTCTTCCACAGACTGGAAGAAAGCTTCCTGGTCGAAGAAGACAAGAAGCACGAAAGACACCCGATC | |
| for use with | TTCGGAAACATCGTCGACGAAGTCGCATACCACGAAAAGTACCCGACAATCTACCACCTGAGAAAGAAGCTGGTCGACAGCACAGA | |
| CleanCap™, | CAAGGCAGACCTGAGACTGATCTACCTGGCACTGGCACACATGATCAAGTTCAGAGGACACTTCCTGATCGAAGGAGACCTGAACC | |
| 5′ UTR of | CGGACAACAGCGACGTCGACAAGCTGTTCATCCAGCTGGTCCAGACATACAACCAGCTGTTCGAAGAAAACCCGATCAACGCAAGC | |
| HSD, ORF | GGAGTCGACGCAAAGGCAATCCTGAGCGCAAGACTGAGCAAGAGCAGAAGACTGGAAAACCTGATCGCACAGCTGCCGGGAGAAAA | |
| corresponding | GAAGAACGGACTGTTCGGAAACCTGATCGCACTGAGCCTGGGACTGACACCGAACTTCAAGAGCAACTTCGACCTGGCAGAAGACG | |
| to SEQ ID | CAAAGCTGCAGCTGAGCAAGGACACATACGACGACGACCTGGACAACCTGCTGGCACAGATCGGAGACCAGTACGCAGACCTGTTC | |
| NO: 4, Kozak | CTGGCAGCAAAGAACCTGAGCGACGCAATCCTGCTGAGCGACATCCTGAGAGTCAACACAGAAATCACAAAGGCACCGCTGAGCGC | |
| sequence, | AAGCATGATCAAGAGATACGACGAACACCACCAGGACCTGACACTGCTGAAGGCACTGGTCAGACAGCAGCTGCCGGAAAAGTACA | |
| and 3′ UTR | AGGAAATCTTCTTCGACCAGAGCAAGAACGGATACGCAGGATACATCGACGGAGGAGCAAGCCAGGAAGAATTCTACAAGTTCATC | |
| of ALB | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCT | |
| | AGCTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| Cas9 | GGGCAGATCGCCTGGAGACGCCATCCACGCTGTTTTGACCTCCATAGAAGACACCGGGACCGATCCAGCCTCCGCGGCCGGGAACG | 257 |
| transcript | GTGCATTGGAACGCGGATTCCCCGTGCCAAGAGTGACTCACCGTCCTTGACACGGCCACCATGGACAAGAAGTACAGCATCGGACT | |
| with 5′ UTR | GGACATCGGAACAAACAGCGTCGGATGGGCAGTCATCACAGACGAATACAAGGTCCCGAGCAAGAAGTTCAAGGTCCTGGGAAACA | |
| from CMV, ORF | CAGACAGACACAGCATCAAGAAGAACCTGATCGGAGCACTGCTGTTCGACAGCGGAGAAACAGCAGAAGCAACAAGACTGAAGAGA | |
| corresponding | ACAGCAAGAAGAAGATACACAAGAAGAAAGAACAGAATCTGCTACCTGCAGGAAATCTTCAGCAACGAAATGGCAAAGGTCGACGA | |
| to SEQ ID | CAGCTTCTTCCACAGACTGGAAGAAAGCTTCCTGGTCGAAGAAGACAAGAAGCACGAAAGACACCCGATCTTCGGAAACATCGTCG | |
| NO: 4, Kozak | ACGAAGTCGCATACCACGAAAAGTACCCGACAATCTACCACCTGAGAAAGAAGCTGGTCGACAGCACAGACAAGGCAGACCTGAGA | |
| sequence, | CTGATCTACCTGGCACTGGCACACATGATCAAGTTCAGAGGACACTTCCTGATCGAAGGAGACCTGAACCCGGACAACAGCGACGT | |
| and 3′ UTR | CGACAAGCTGTTCATCCAGCTGGTCCAGACATACAACCAGCTGTTCGAAGAAAACCCGATCAACGCAAGCGGAGTCGACGCAAAGG | |
| of ALB | CAATCCTGAGCGCAAGACTGAGCAAGAGCAGAAGACTGGAAAACCTGATCGCACAGCTGCCGGGAGAAAAGAAGAACGGACTGTTC | |
| | GGAAACCTGATCGCACTGAGCCTGGGACTGACACCGAACTTCAAGAGCAACTTCGACCTGGCAGAAGACGCAAAGCTGCAGCTGAG | |
| | CAAGGACACATACGACGACGACCTGGACAACCTGCTGGCACAGATCGGAGACCAGTACGCAGACCTGTTCCTGGCAGCAAAGAACC | |
| | TGAGCGACGCAATCCTGCTGAGCGACATCCTGAGAGTCAACACAGAAATCACAAAGGCACCGCTGAGCGCAAGCATGATCAAGAGA | |
| | TACGACGAACACCACCAGGACCTGACACTGCTGAAGGCACTGGTCAGACAGCAGCTGCCGGAAAAGTACAAGGAAATCTTCTTCGA | |
| | CCAGAGCAAGAACGGATACGCAGGATACATCGACGGAGGAGCAAGCCAGGAAGAATTCTACAAGTTCATCAAGCCGATCCTGGAAA | |
| | AGATGGACGGAAGAGAAGAACTGCTGGTCAAGCTGAACAGAGAAGACCTGCTGAGAAAGCAGAGAACATTCGACAACGGAAGCATC | |
| | CCGCACCAGATCCACCTGGGAGAACTGCACGCAATCCTGAGAAGACAGGAAGACTTCTACCCGTTCCTGAAGGACAACAGAGAAAA | |
| | GATCGAAAAGATCCTGACATTCAGAATCCCGTACTACGTCGGACCGCTGGCAAGAGGAAACAGCAGATTCGCATGGATGACAAGAA | |
| | AGAGCGAAGAAACAATCACACCGTGGAACTTCGAAGAAGTCGTCGACAAGGGAGCAAGCGCACAGAGCTTCATCGAAAGAATGACA | |
| | AACTTCGACAAGAACCTGCCGAACGAAAAGGTCCTGCCGAAGCACAGCCTGCTGTACGAATACTTCACAGTCTACAACGAACTGAC | |
| | AAAGGTCAAGTACGTCACAGAAGGAATGAGAAAGCCGGCATTCCTGAGCGGAGAACAGAAGAAGGCAATCGTCGACCTGCTGTTCA | |
| | AGACAAACAGAAAGGTCACAGTCAAGCAGCTGAAGGAAGACTACTTCAAGAAGATCGAATGCTTCGACAGCGTCGAAATCAGCGGA | |
| | GTCGAAGACAGATTCAACGCAAGCCTGGGAACATACCACGACCTGCTGAAGATCATCAAGGACAAGGACTTCCTGGACAACGAAGA | |
| | AAACGAAGACATCCTGGAAGACATCGTCCTGACACTGACACTGTTCGAAGACAGAGAAATGATCGAAGAAAGACTGAAGACATACG | |
| | CACACCTGTTCGACGACAAGGTCATGAAGCAGCTGAAGAGAAGAAGATACACAGGATGGGGAAGACTGAGCAGAAAGCTGATCAAC | |
| | GGAATCAGAGACAAGCAGAGCGGAAAGACAATCCTGGACTTCCTGAAGAGCGACGGATTCGCAAACAGAAACTTCATGCAGCTGAT | |
| | CCACGACGACAGCCTGACATTCAAGGAAGACATCCAGAAGGCACAGGTCAGCGGACAGGGAGACAGCCTGCACGAACACATCGCAA | |
| | ACCTGGCAGGAAGCCCGGCAATCAAGAAGGGAATCCTGCAGACAGTCAAGGTCGTCGACGAACTGGTCAAGGTCATGGGAAGACAC | |
| | AAGCCGGAAAACATCGTCATCGAAATGGCAAGAGAAAACCAGACAACACAGAAGGGACAGAAGAACAGCAGAGAAAGAATGAAGAG | |
| | AATCGAAGAAGGAATCAAGGAACTGGGAAGCCAGATCCTGAAGGAACACCCGGTCGAAAACACACAGCTGCAGAACGAAAAGCTGT | |
| | ACCTGTACTACCTGCAGAACGGAAGAGACATGTACGTCGACCAGGAACTGGACATCAACAGACTGAGCGACTACGACGTCGACCAC | |
| | ATCGTCCCGCAGAGCTTCCTGAAGGACGACAGCATCGACAACAAGGTCCTGACAAGAAGCGACAAGAACAGAGGAAAGAGCGACAA | |
| | CGTCCCGAGCGAAGAAGTCGTCAAGAAGATGAAGAACTACTGGAGACAGCTGCTGAACGCAAAGCTGATCACACAGAGAAAGTTCG | |
| | ACAACCTGACAAAGGCAGAGAGAGGAGGACTGAGCGAACTGGACAAGGCAGGATTCATCAAGAGACAGCTGGTCGAAACAAGACAG | |
| | ATCACAAAGCACGTCGCACAGATCCTGGACAGCAGAATGAACACAAAGTACGACGAAAACGACAAGCTGATCAGAGAAGTCAAGGT | |
| | CATCACACTGAAGAGCAAGCTGGTCAGCGACTTCAGAAAGGACTTCCAGTTCTACAAGGTCAGAGAAATCAACAACTACCACCACG | |
| | CACACGACGCATACCTGAACGCAGTCGTCGGAACAGCACTGATCAAGAAGTACCCGAAGCTGGAAAGCGAATTCGTCTACGGAGAC | |
| | TACAAGGTCTACGACGTCAGAAAGATGATCGCAAAGAGCGAACAGGAAATCGGAAAGGCAACAGCAAAGTACTTCTTCTACAGCAA | |
| | CATCATGAACTTCTTCAAGACAGAAATCACACTGGCAAACGGAGAAATCAGAAAGAGACCGCTGATCGAAACAAACGGAGAAACAG | |
| | GAGAAATCGTCTGGGACAAGGGAAGAGACTTCGCAACAGTCAGAAAGGTCCTGAGCATGCCGCAGGTCAACATCGTCAAGAAGACA | |
| | GAAGTCCAGACAGGAGGATTCAGCAAGGAAAGCATCCTGCCGAAGAGAAACAGCGACAAGCTGATCGCAAGAAAGAAGGACTGGGA | |
| | CCCGAAGAAGTACGGAGGATTCGACAGCCCGACAGTCGCATACAGCGTCCTGGTCGTCGCAAAGGTCGAAAAGGGAAAGAGCAAGA | |
| | AGCTGAAGAGCGTCAAGGAACTGCTGGGAATCACAATCATGGAAAGAAGCAGCTTCGAAAAGAACCCGATCGACTTCCTGGAAGCA | |
| | AAGGGATACAAGGAAGTCAAGAAGGACCTGATCATCAAGCTGCCGAAGTACAGCCTGTTCGAACTGGAAAACGGAAGAAAGAGAAT | |
| | GCTGGCAAGCGCAGGAGAACTGCAGAAGGGAAACGAACTGGCACTGCCGAGCAAGTACGTCAACTTCCTGTACCTGGCAAGCCACT | |
| | ACGAAAAGCTGAAGGGAAGCCCGGAAGACAACGAACAGAAGCAGCTGTTCGTCGAACAGCACAAGCACTACCTGGACGAAATCATC | |
| | GAACAGATCAGCGAATTCAGCAAGAGAGTCATCCTGGCAGACGCAAACCTGGACAAGGTCCTGAGCGCATACAACAAGCACAGAGA | |
| | CAAGCCGATCAGAGAACAGGCAGAAAACATCATCCACCTGTTCACACTGACAAACCTGGGAGCACCGGCAGCATTCAAGTACTTCG | |
| | ACACAACAATCGACAGAAAGAGATACACAAGCACAAAGGAAGTCCTGGACGCAACACTGATCCACCAGAGCATCACAGGACTGTAC | |
| | GAAACAAGAATCGACCTGAGCCAGCTGGGAGGAGACGGAGGAGGAAGCCCGAAGAAGAAGAGAAAGGTCTAGCTAGCCATCACATT | |
| | TAAAAGCATCTCAGCCTACCATGAGAATAAGAGAAAGAAAATGAAGATCAATAGCTTATTCATCTCTTTTTCTTTTTCGTTGGTGT | |
| | AAAGCCAACACCCTGTCTAAAAAACATAAATTTCTTTAATCATTTTGCCTCTTTTCTCTGTGCTTCAATTAATAAAAAATGGAAAG | |
| | AACCTCGAG | |
| Cas9 | GGGacatttgcttctgacacaactgtgttcactagcaacctcaaacagacaccggatctgccaccATGGACAAGAAGTACAGCATC | 258 |
| transcript | GGACTGGACATCGGAACAAACAGCGTCGGATGGGCAGTCATCACAGACGAATACAAGGTCCCGAGCAAGAAGTTCAAGGTCCTGGG | |
| with 5′ UTR | AAACACAGACAGACACAGCATCAAGAAGAACCTGATCGGAGCACTGCTGTTCGACAGCGGAGAAACAGCAGAAGCAACAAGACTGA | |
| from HBB, ORF | AGAGAACAGCAAGAAGAAGATACACAAGAAGAAAGAACAGAATCTGCTACCTGCAGGAAATCTTCAGCAACGAAATGGCAAAGGTC | |
| corresponding | GACGACAGCTTCTTCCACAGACTGGAAGAAAGCTTCCTGGTCGAAGAAGACAAGAAGCACGAAAGACACCCGATCTTCGGAAACAT | |
| to SEQ ID | CGTCGACGAAGTCGCATACCACGAAAAGTACCCGACAATCTACCACCTGAGAAAGAAGCTGGTCGACAGCACAGACAAGGCAGACC | |
| NO: 4, Kozak | TGAGACTGATCTACCTGGCACTGGCACACATGATCAAGTTCAGAGGACACTTCCTGATCGAAGGAGACCTGAACCCGGACAACAGC | |
| sequence, | GACGTCGACAAGCTGTTCATCCAGCTGGTCCAGACATACAACCAGCTGTTCGAAGAAAACCCGATCAACGCAAGCGGAGTCGACGC | |
| and 3′ UTR | AAAGGCAATCCTGAGCGCAAGACTGAGCAAGAGCAGAAGACTGGAAAACCTGATCGCACAGCTGCCGGGAGAAAAGAAGAACGGAC | |
| of HBB | TGTTCGGAAACCTGATCGCACTGAGCCTGGGACTGACACCGAACTTCAAGAGCAACTTCGACCTGGCAGAAGACGCAAAGCTGCAG | |
| | CTGAGCAAGGACACATACGACGACGACCTGGACAACCTGCTGGCACAGATCGGAGACCAGTACGCAGACCTGTTCCTGGCAGCAAA | |
| | GAACCTGAGCGACGCAATCCTGCTGAGCGACATCCTGAGAGTCAACACAGAAATCACAAAGGCACCGCTGAGCGCAAGCATGATCA | |
| | AGAGATACGACGAACACCACCAGGACCTGACACTGCTGAAGGCACTGGTCAGACAGCAGCTGCCGGAAAAGTACAAGGAAATCTTC | |
| | TTCGACCAGAGCAAGAACGGATACGCAGGATACATCGACGGAGGAGCAAGCCAGGAAGAATTCTACAAGTTCATCAAGCCGATCCT | |
| | GGAAAAGATGGACGGAACAGAAGAACTGCTGGTCAAGCTGAACAGAGAAGACCTGCTGAGAAAGCAGAGAACATTCGACAACGGAA | |
| | GCATCCCGCACCAGATCCACCTGGGAGAACTGCACGCAATCCTGAGAAGACAGGAAGACTTCTACCCGTTCCTGAAGGACAACAGA | |
| | GAAAAGATCGAAAAGATCCTGACATTCAGAATCCCGTACTACGTCGGACCGCTGGCAAGAGGAAACAGCAGATTCGCATGGATGAC | |
| | AAGAAAGAGCGAAGAAACAATCACACCGTGGAACTTCGAAGAAGTCGTCGACAAGGGAGCAAGCGCACAGAGCTTCATCGAAAGAA | |
| | TGACAAACTTCGACAAGAACCTGCCGAACGAAAAGGTCCTGCCGAAGCACAGCCTGCTGTACGAATACTTCACAGTCTACAACGAA | |
| | CTGACAAAGGTCAAGTACGTCACAGAAGGAATGAGAAAGCCGGCATTCCTGAGCGGAGAACAGAAGAAGGCAATCGTCGACCTGCT | |
| | GTTCAAGACAAACAGAAAGGTCACAGTCAAGCAGCTGAAGGAAGACTACTTCAAGAAGATCGAATGCTTCGACAGCGTCGAAATCA | |
| | GCGGAGTCGAAGACAGATTCAACGCAAGCCTGGGAACATACCACGACCTGCTGAAGATCATCAAGGACAAGGACTTCCTGGACAAC | |
| | GAAGAAAACGAAGACATCCTGGAAGACATCGTCCTGACACTGACACTGTTCGAAGACAGAGAAATGATCGAAGAAAGACTGAAGAC | |
| | ATACGCACACCTGTTCGACGACAAGGTCATGAAGCAGCTGAAGAGAAGAAGATACACAGGATGGGGAAGACTGAGCAGAAAGCTGA | |
| | TCAACGGAATCAGAGACAAGCAGAGCGGAAAGACAATCCTGGACTTCCTGAAGAGCGACGGATTCGCAAACAGAAACTTCATGCAG | |
| | CTGATCCACGACGACAGCCTGACATTCAAGGAAGACATCCAGAAGGCACAGGTCAGCGGACAGGGAGACAGCCTGCACGAACACAT | |
| | CGCAAACCTGGCAGGAAGCCCGGCAATCAAGAAGGGAATCCTGCAGACAGTCAAGGTCGTCGACGAACTGGTCAAGGTCATGGGAA | |
| | GACACAAGCCGGAAAACATCGTCATCGAAATGGCAAGAGAAAACCAGACAACACAGAAGGGACAGAAGAACAGCAGAGAAAGAATG | |
| | AAGAGAATCGAAGAAGGAATCAAGGAACTGGGAAGCCAGATCCTGAAGGAACACCCGGTCGAAAACACACAGCTGCAGAACGAAAA | |
| | GCTGTACCTGTACTACCTGCAGAACGGAAGAGACATGTACGTCGACCAGGAACTGGACATCAACAGACTGAGCGACTACGACGTCG | |
| | ACCACATCGTCCCGCAGAGCTTCCTGAAGGACGACAGCATCGACAACAAGGTCCTGACAAGAAGCGACAAGAACAGAGGAAAGAGC | |
| | GACAACGTCCCGAGCGAAGAAGTCGTCAAGAAGATGAAGAACTACTGGAGACAGCTGCTGAACGCAAAGCTGATCACACAGAGAAA | |
| | GTTCGACAACCTGACAAAGGCAGAGAGAGGAGGACTGAGCGAACTGGACAAGGCAGGATTCATCAAGAGACAGCTGGTCGAAACAA | |
| | GACAGATCACAAAGCACGTCGCACAGATCCTGGACAGCAGAATGAACACAAAGTACGACGAAAACGACAAGCTGATCAGAGAAGTC | |
| | AAGGTCATCACACTGAAGAGCAAGCTGGTCAGCGACTTCAGAAAGGACTTCCAGTTCTACAAGGTCAGAGAAATCAACAACTACCA | |
| | CCACGCACACGACGCATACCTGAACGCAGTCGTCGGAACAGCACTGATCAAGAAGTACCCGAAGCTGGAAAGCGAATTCGTCTACG | |
| | GAGACTACAAGGTCTACGACGTCAGAAAGATGATCGCAAAGAGCGAACAGGAAATCGGAAAGGCAACAGCAAAGTACTTCTTCTAC | |
| | AGCAACATCATGAACTTCTTCAAGACAGAAATCACACTGGCAAACGGAGAAATCAGAAAGAGACCGCTGATCGAAACAAACGGAGA | |
| | AACAGGAGAAATCGTCTGGGACAAGGGAAGAGACTTCGCAACAGTCAGAAAGGTCCTGAGCATGCCGCAGGTCAACATCGTCAAGA | |
| | AGACAGAAGTCCAGACAGGAGGATTCAGCAAGGAAAGCATCCTGCCGAAGAGAAACAGCGACAAGCTGATCGCAAGAAAGAAGGAC | |
| | TGGGACCCGAAGAAGTACGGAGGATTCGACAGCCCGACAGTCGCATACAGCGTCCTGGTCGTCGCAAAGGTCGAAAAGGGAAAGAG | |
| | CAAGAAGCTGAAGAGCGTCAAGGAACTGCTGGGAATCACAATCATGGAAAGAAGCAGCTTCGAAAAGAACCCGATCGACTTCCTGG | |
| | AAGCAAAGGGATACAAGGAAGTCAAGAAGGACCTGATCATCAAGCTGCCGAAGTACAGCCTGTTCGAACTGGAAAACGGAAGAAAG | |
| | AGAATGCTGGCAAGCGCAGGAGAACTGCAGAAGGGAAACGAACTGGCACTGCCGAGCAAGTACGTCAACTTCCTGTACCTGGCAAG | |
| | CCACTACGAAAAGCTGAAGGGAAGCCCGGAAGACAACGAACAGAAGCAGCTGTTCGTCGAACAGCACAAGCACTACCTGGACGAAA | |
| | TCATCGAACAGATCAGCGAATTCAGCAAGAGAGTCATCCTGGCAGACGCAAACCTGGACAAGGTCCTGAGCGCATACAACAAGCAC | |
| | AGAGACAAGCCGATCAGAGAACAGGCAGAAAACATCATCCACCTGTTCACACTGACAAACCTGGGAGCACCGGCAGCATTCAAGTA | |
| | CTTCGACACAACAATCGACAGAAAGAGATACACAAGCACAAAGGAAGTCCTGGACGCAACACTGATCCACCAGAGCATCACAGGAC | |
| | TGTACGAAACAAGAATCGACCTGAGCCAGCTGGGAGGAGACGGAGGAGGAAGCCCGAAGAAGAAGAGAAAGGTCTAGctagcgctc | |
| | gctttcttgctgtccaatttctattaaaggttcctttgttccctaagtccaactactaaactgggggatattatgaagggccttga | |
| | gcatctggattctgcctaataaaaaacatttattttcattgcctcgag | |
| Cas9 | GGGaagctcagaataaacgctcaactttggccggatctgccacCATGGACAAGAAGTACAGCATCGGACTGGACATCGGAACAAAC | 259 |
| transcript | AGCGTCGGATGGGCAGTCATCACAGACGAATACAAGGTCCCGAGCAAGAAGTTCAAGGTCCTGGGAAACACAGACAGACACAGCAT | |
| with 5′ UTR | CAAGAAGAACCTGATCGGAGCACTGCTGTTCGACAGCGGAGAAACAGCAGAAGCAACAAGACTGAAGAGAACAGCAAGAAGAAGAT | |
| from XBG, | ACACAAGAAGAAAGAACAGAATCTGCTACCTGCAGGAAATCTTCAGCAACGAAATGGCAAAGGTCGACGACAGCTTCTTCCACAGA | |
| ORF | CTGGAAGAAAGCTTCCTGGTCGAAGAAGACAAGAAGCACGAAAGACACCCGATCTTCGGAAACATCGTCGACGAAGTCGCATACCA | |
| corresponding | CGAAAAGTACCCGACAATCTACCACCTGAGAAAGAAGCTGGTCGACAGCACAGACAAGGCAGACCTGAGACTGATCTACCTGGCAC | |
| to SEQ ID | TGGCACACATGATCAAGTTCAGAGGACACTTCCTGATCGAAGGAGACCTGAACCCGGACAACAGCGACGTCGACAAGCTGTTCATC | |
| NO: 4, Kozak | CAGCTGGTCCAGACATACAACCAGCTGTTCGAAGAAAACCCGATCAACGCAAGCGGAGTCGACGCAAAGGCAATCCTGAGCGCAAG | |
| sequence, | ACTGAGCAAGAGCAGAAGACTGGAAAACCTGATCGCACAGCTGCCGGGAGAAAAGAAGAACGGACTGTTCGGAAACCTGATCGCAC | |
| and 3′ UTR | TGAGCCTGGGACTGACACCGAACTTCAAGAGCAACTTCGACCTGGCAGAAGACGCAAAGCTGCAGCTGAGCAAGGACACATACGAC | |
| of XBG | GACGACCTGGACAACCTGCTGGCACAGATCGGAGACCAGTACGCAGACCTGTTCCTGGCAGCAAAGAACCTGAGCGACGCAATCCT | |
| | GCTGAGCGACATCCTGAGAGTCAACACAGAAATCACAAAGGCACCGCTGAGCGCAAGCATGATCAAGAGATACGACGAACACCACC | |
| | AGGACCTGACACTGCTGAAGGCACTGGTCAGACAGCAGCTGCCGGAAAAGTACAAGGAAATCTTCTTCGACCAGAGCAAGAACGGA | |
| | TACGCAGGATACATCGACGGAGGAGCAAGCCAGGAAGAATTCTACAAGTTCATCAAGCCGATCCTGGAAAAGATGGACGGAACAGA | |
| | AGAACTGCTGGTCAAGCTGAACAGAGAAGACCTGCTGAGAAAGCAGAGAACATTCGACAACGGAAGCATCCCGCACCAGATCCACC | |
| | TGGGAGAACTGCACGCAATCCTGAGAAGACAGGAAGACTTCTACCCGTTCCTGAAGGACAACAGAGAAAAGATCGAAAAGATCCTG | |
| | ACATTCAGAATCCCGTACTACGTCGGACCGCTGGCAAGAGGAAACAGCAGATTCGCATGGATGACAAGAAAGAGCGAAGAAACAAT | |
| | CACACCGTGGAACTTCGAAGAAGTCGTCGACAAGGGAGCAAGCGCACAGAGCTTCATCGAAAGAATGACAAACTTCGACAAGAACC | |
| | TGCCGAACGAAAAGGTCCTGCCGAAGCACAGCCTGCTGTACGAATACTTCACAGTCTACAACGAACTGACAAAGGTCAAGTACGTC | |
| | ACAGAAGGAATGAGAAAGCCGGCATTCCTGAGCGGAGAACAGAAGAAGGCAATCGTCGACCTGCTGTTCAAGACAAACAGAAAGGT | |
| | CACAGTCAAGCAGCTGAAGGAAGACTACTTCAAGAAGATCGAATGCTTCGACAGCGTCGAAATCAGCGGAGTCGAAGACAGATTCA | |
| | ACGCAAGCCTGGGAACATACCACGACCTGCTGAAGATCATCAAGGACAAGGACTTCCTGGACAACGAAGAAAACGAAGACATCCTG | |
| | GAAGACATCGTCCTGACACTGACACTGTTCGAAGACAGAGAAATGATCGAAGAAAGACTGAAGACATACGCACACCTGTTCGACGA | |
| | CAAGGTCATGAAGCAGCTGAAGAGAAGAAGATACACAGGATGGGGAAGACTGAGCAGAAAGCTGATCAACGGAATCAGAGACAAGC | |
| | AGAGCGGAAAGACAATCCTGGACTTCCTGAAGAGCGACGGATTCGCAAACAGAAACTTCATGCAGCTGATCCACGACGACAGCCTG | |
| | ACATTCAAGGAAGACATCCAGAAGGCACAGGTCAGCGGACAGGGAGACAGCCTGCACGAACACATCGCAAACCTGGCAGGAAGCCC | |
| | GGCAATCAAGAAGGGAATCCTGCAGACAGTCAAGGTCGTCGACGAACTGGTCAAGGTCATGGGAAGACACAAGCCGGAAAACATCG | |
| | TCATCGAAATGGCAAGAGAAAACCAGACAACACAGAAGGGACAGAAGAACAGCAGAGAAAGAATGAAGAGAATCGAAGAAGGAATC | |
| | AAGGAACTGGGAAGCCAGATCCTGAAGGAACACCCGGTCGAAAACACACAGCTGCAGAACGAAAAGCTGTACCTGTACTACCTGCA | |
| | GAACGGAAGAGACATGTACGTCGACCAGGAACTGGACATCAACAGACTGAGCGACTACGACGTCGACCACATCGTCCCGCAGAGCT | |
| | TCCTGAAGGACGACAGCATCGACAACAAGGTCCTGACAAGAAGCGACAAGAACAGAGGAAAGAGCGACAACGTCCCGAGCGAAGAA | |
| | GTCGTCAAGAAGATGAAGAACTACTGGAGACAGCTGCTGAACGCAAAGCTGATCACACAGAGAAAGTTCGACAACCTGACAAAGGC | |
| | AGAGAGAGGAGGACTGAGCGAACTGGACAAGGCAGGATTCATCAAGAGACAGCTGGTCGAAACAAGACAGATCACAAAGCACGTCG | |
| | CACAGATCCTGGACAGCAGAATGAACACAAAGTACGACGAAAACGACAAGCTGATCAGAGAAGTCAAGGTCATCACACTGAAGAGC | |
| | AAGCTGGTCAGCGACTTCAGAAAGGACTTCCAGTTCTACAAGGTCAGAGAAATCAACAACTACCACCACGCACACGACGCATACCT | |
| | GAACGCAGTCGTCGGAACAGCACTGATCAAGAAGTACCCGAAGCTGGAAAGCGAATTCGTCTACGGAGACTACAAGGTCTACGACG | |
| | TCAGAAAGATGATCGCAAAGAGCGAACAGGAAATCGGAAAGGCAACAGCAAAGTACTTCTTCTACAGCAACATCATGAACTTCTTC | |
| | AAGACAGAAATCACACTGGCAAACGGAGAAATCAGAAAGAGACCGCTGATCGAAACAAACGGAGAAACAGGAGAAATCGTCTGGGA | |
| | CAAGGGAAGAGACTTCGCAACAGTCAGAAAGGTCCTGAGCATGCCGCAGGTCAACATCGTCAAGAAGACAGAAGTCCAGACAGGAG | |
| | GATTCAGCAAGGAAAGCATCCTGCCGAAGAGAAACAGCGACAAGCTGATCGCAAGAAAGAAGGACTGGGACCCGAAGAAGTACGGA | |
| | GGATTCGACAGCCCGACAGTCGCATACAGCGTCCTGGTCGTCGCAAAGGTCGAAAAGGGAAAGAGCAAGAAGCTGAAGAGCGTCAA | |
| | GGAACTGCTGGGAATCACAATCATGGAAAGAAGCAGCTTCGAAAAGAACCCGATCGACTTCCTGGAAGCAAAGGGATACAAGGAAG | |
| | TCAAGAAGGACCTGATCATCAAGCTGCCGAAGTACAGCCTGTTCGAACTGGAAAACGGAAGAAAGAGAATGCTGGCAAGCGCAGGA | |
| | GAACTGCAGAAGGGAAACGAACTGGCACTGCCGAGCAAGTACGTCAACTTCCTGTACCTGGCAAGCCACTACGAAAAGCTGAAGGG | |
| | AAGCCCGGAAGACAACGAACAGAAGCAGCTGTTCGTCGAACAGCACAAGCACTACCTGGACGAAATCATCGAACAGATCAGCGAAT | |
| | TCAGCAAGAGAGTCATCCTGGCAGACGCAAACCTGGACAAGGTCCTGAGCGCATACAACAAGCACAGAGACAAGCCGATCAGAGAA | |
| | CAGGCAGAAAACATCATCCACCTGTTCACACTGACAAACCTGGGAGCACCGGCAGCATTCAAGTACTTCGACACAACAATCGACAG | |
| | AAAGAGATACACAAGCACAAAGGAAGTCCTGGACGCAACACTGATCCACCAGAGCATCACAGGACTGTACGAAACAAGAATCGACC | |
| | TGAGCCAGCTGGGAGGAGACGGAGGAGGAAGCCCGAAGAAGAAGAGAAAGGTCTAGctagcaccagcctcaagaacacccgaatgg | |
| | agtctctaagctacataataccaacttacactttacaaaatgttgtcccccaaaatgtagccattcgtatctgctcctaataaaaa | |
| | gaaagtttcttcacattctctcgag | |
| Cas9 | AGGaagctcagaataaacgctcaactttggccggatctgccacCATGGACAAGAAGTACAGCATCGGACTGGACATCGGAACAAAC | 260 |
| transcript | AGCGTCGGATGGGCAGTCATCACAGACGAATACAAGGTCCCGAGCAAGAAGTTCAAGGTCCTGGGAAACACAGACAGACACAGCAT | |
| with AGG as | CAAGAAGAACCTGATCGGAGCACTGCTGTTCGACAGCGGAGAAACAGCAGAAGCAACAAGACTGAAGAGAACAGCAAGAAGAAGAT | |
| first three | ACACAAGAAGAAAGAACAGAATCTGCTACCTGCAGGAAATCTTCAGCAACGAAATGGCAAAGGTCGACGACAGCTTCTTCCACAGA | |
| nucleotides | CTGGAAGAAAGCTTCCTGGTCGAAGAAGACAAGAAGCACGAAAGACACCCGATCTTCGGAAACATCGTCGACGAAGTCGCATACCA | |
| for use with | CGAAAAGTACCCGACAATCTACCACCTGAGAAAGAAGCTGGTCGACAGCACAGACAAGGCAGACCTGAGACTGATCTACCTGGCAC | |
| CleanCap ™, | TGGCACACATGATCAAGTTCAGAGGACACTTCCTGATCGAAGGAGACCTGAACCCGGACAACAGCGACGTCGACAAGCTGTTCATC | |
| 5′ UTR from | CAGCTGGTCCAGACATACAACCAGCTGTTCGAAGAAAACCCGATCAACGCAAGCGGAGTCGACGCAAAGGCAATCCTGAGCGCAAG | |
| XBG, ORF | ACTGAGCAAGAGCAGAAGACTGGAAAACCTGATCGCACAGCTGCCGGGAGAAAAGAAGAACGGACTGTTCGGAAACCTGATCGCAC | |
| corresponding | TGAGCCTGGGACTGACACCGAACTTCAAGAGCAACTTCGACCTGGCAGAAGACGCAAAGCTGCAGCTGAGCAAGGACACATACGAC | |
| to SEQ ID | GACGACCTGGACAACCTGCTGGCACAGATCGGAGACCAGTACGCAGACCTGTTCCTGGCAGCAAAGAACCTGAGCGACGCAATCCT | |
| NO: 4, Kozak | GCTGAGCGACATCCTGAGAGTCAACACAGAAATCACAAAGGCACCGCTGAGCGCAAGCATGATCAAGAGATACGACGAACACCACC | |
| sequence, | AGGACCTGACACTGCTGAAGGCACTGGTCAGACAGCAGCTGCCGGAAAAGTACAAGGAAATCTTCTTCGACCAGAGCAAGAACGGA | |
| and 3′ UTR | TACGCAGGATACATCGACGGAGGAGCAAGCCAGGAAGAATTCTACAAGTTCATCAAGCCGATCCTGGAAAAGATGGACGGAACAGA | |
| of XBG | AGAACTGCTGGTCAAGCTGAACAGAGAAGACCTGCTGAGAAAGCAGAGAACATTCGACAACGGAAGCATCCCGCACCAGATCCACC | |
| | TGGGAGAACTGCACGCAATCCTGAGAAGACAGGAAGACTTCTACCCGTTCCTGAAGGACAACAGAGAAAAGATCGAAAAGATCCTG | |
| | ACATTCAGAATCCCGTACTACGTCGGACCGCTGGCAAGAGGAAACAGAAGATTCGCATGGATGACAAGAAAGAGCGAAGAAACAAT | |
| | CACACCGTGGAACTTCGAAGAAGTCGTCGACAAGGGAGCAAGCGCACAGAGCTTCATCGAAAGAATGACAAACTTCGACAAGAACC | |
| | TGCCGAACGAAAAGGTCCTGCCGAAGCACAGCCTGCTGTACGAATACTTCACAGTCTACAACGAACTGACAAAGGTCAAGTACGTC | |
| | ACAGAAGGAATGAGAAAGCCGGCATTCCTGAGCGGAGAACAGAAGAAGGCAATCGTCGACCTGCTGTTCAAGACAAACAGAAAGGT | |
| | CACAGTCAAGCAGCTGAAGGAAGACTACTTCAAGAAGATCGAATGCTTCGACAGCGTCGAAATCAGCGGAGTCGAAGACAGATTCA | |
| | ACGCAAGCCTGGGAACATACCACGACCTGCTGAAGATCATCAAGGACAAGGACTTCCTGGACAACGAAGAAAACGAAGACATCCTG | |
| | GAAGACATCGTCCTGACACTGACACTGTTCGAAGACAGAGAAATGATCGAAGAAAGACTGAAGACATACGCACACCTGTTCGACGA | |
| | CAAGGTCATGAAGCAGCTGAAGAGAAGAAGATACACAGGATGGGGAAGACTGAGCAGAAAGCTGATCAACGGAATCAGAGACAAGC | |
| | AGAGCGGAAAGACAATCCTGGACTTCCTGAAGAGCGACGGATTCGCAAACAGAAACTTCATGCAGCTGATCCACGACGACAGCCTG | |
| | ACATTCAAGGAAGACATCCAGAAGGCACAGGTCAGCGGACAGGGAGACAGCCTGCACGAACACATCGCAAACCTGGCAGGAAGCCC | |
| | GGCAATCAAGAAGGGAATCCTGCAGACAGTCAAGGTCGTCGACGAACTGGTCAAGGTCATGGGAAGACACAAGCCGGAAAACATCG | |
| | TCATCGAAATGGCAAGAGAAAACCAGACAACACAGAAGGGACAGAAGAACAGCAGAGAAAGAATGAAGAGAATCGAAGAAGGAATC | |
| | AAGGAACTGGGAAGCCAGATCCTGAAGGAACACCCGGTCGAAAACACACAGCTGCAGAACGAAAAGCTGTACCTGTACTACCTGCA | |
| | GAACGGAAGAGACATGTACGTCGACCAGGAACTGGACATCAACAGACTGAGCGACTACGACGTCGACCACATCGTCCCGCAGAGCT | |
| | TCCTGAAGGACGACAGCATCGACAACAAGGTCCTGACAAGAAGCGACAAGAACAGAGGAAAGAGCGACAACGTCCCGAGCGAAGAA | |
| | GTCGTCAAGAAGATGAAGAACTACTGGAGACAGCTGCTGAACGCAAAGCTGATCACACAGAGAAAGTTCGACAACCTGACAAAGGC | |
| | AGAGAGAGGAGGACTGAGCGAACTGGACAAGGCAGGATTCATCAAGAGACAGCTGGTCGAAACAAGACAGATCACAAAGCACGTCG | |
| | CACAGATCCTGGACAGCAGAATGAACACAAAGTACGACGAAAACGACAAGCTGATCAGAGAAGTCAAGGTCATCACACTGAAGAGC | |
| | AAGCTGGTCAGCGACTTCAGAAAGGACTTCCAGTTCTACAAGGTCAGAGAAATCAACAACTACCACCACGCACACGACGCATACCT | |
| | GAACGCAGTCGTCGGAACAGCACTGATCAAGAAGTACCCGAAGCTGGAAAGCGAATTCGTCTACGGAGACTACAAGGTCTACGACG | |
| | TCAGAAAGATGATCGCAAAGAGCGAACAGGAAATCGGAAAGGCAACAGCAAAGTACTTCTTCTACAGCAACATCATGAACTTCTTC | |
| | AAGACAGAAATCACACTGGCAAACGGAGAAATCAGAAAGAGACCGCTGATCGAAACAAACGGAGAAACAGGAGAAATCGTCTGGGA | |
| | CAAGGGAAGAGACTTCGCAACAGTCAGAAAGGTCCTGAGCATGCCGCAGGTCAACATCGTCAAGAAGACAGAAGTCCAGACAGGAG | |
| | GATTCAGCAAGGAAAGCATCCTGCCGAAGAGAAACAGCGACAAGCTGATCGCAAGAAAGAAGGACTGGGACCCGAAGAAGTACGGA | |
| | GGATTCGACAGCCCGACAGTCGCATACAGCGTCCTGGTCGTCGCAAAGGTCGAAAAGGGAAAGAGCAAGAAGCTGAAGAGCGTCAA | |
| | GGAACTGCTGGGAATCACAATCATGGAAAGAAGCAGCTTCGAAAAGAACCCGATCGACTTCCTGGAAGCAAAGGGATACAAGGAAG | |
| | TCAAGAAGGACCTGATCATCAAGCTGCCGAAGTACAGCCTGTTCGAACTGGAAAACGGAAGAAAGAGAATGCTGGCAAGCGCAGGA | |
| | GAACTGCAGAAGGGAAACGAACTGGCACTGCCGAGCAAGTACGTCAACTTCCTGTACCTGGCAAGCCACTACGAAAAGCTGAAGGG | |
| | AAGCCCGGAAGACAACGAACAGAAGCAGCTGTTCGTCGAACAGCACAAGCACTACCTGGACGAAATCATCGAACAGATCAGCGAAT | |
| | TCAGCAAGAGAGTCATCCTGGCAGACGCAAACCTGGACAAGGTCCTGAGCGCATACAACAAGCACAGAGACAAGCCGATCAGAGAA | |
| | CAGGCAGAAAACATCATCCACCTGTTCACACTGACAAACCTGGGAGCACCGGCAGCATTCAAGTACTTCGACACAACAATCGACAG | |
| | AAAGAGATACACAAGCACAAAGGAAGTCCTGGACGCAACACTGATCCACCAGAGCATCACAGGACTGTACGAAACAAGAATCGACC | |
| | TGAGCCAGCTGGGAGGAGACGGAGGAGGAAGCCCGAAGAAGAAGAGAAAGGTCTAGctagcaccagcctcaagaacacccgaatgg | |
| | agtctctaagctacataataccaacttacactttacaaaatgttgtcccccaaaatgtagccattcgtatctgctcctaataaaaa | |
| | gaaagtttcttcacattctctcgag | |
| Cas9 | AGGTCCCGCAGTCGGCGTCCAGCGGCTCTGCTTGTTCGTGTGTGTGTCGTTGCAGGCCTTATTCGGATCCGCCACCATGGACAAGA | 261 |
| transcript | AGTACAGCATCGGACTGGACATCGGAACAAACAGCGTCGGATGGGCAGTCATCACAGACGAATACAAGGTCCCGAGCAAGAAGTTC | |
| with AGG as | AAGGTCCTGGGAAACACAGACAGACACAGCATCAAGAAGAACCTGATCGGAGCACTGCTGTTCGACAGCGGAGAAACAGCAGAAGC | |
| first three | AACAAGACTGAAGAGAACAGCAAGAAGAAGATACACAAGAAGAAAGAACAGAATCTGCTACCTGCAGGAAATCTTCAGCAACGAAA | |
| nucleotides | TGGCAAAGGTCGACGACAGCTTCTTCCACAGACTGGAAGAAAGCTTCCTGGTCGAAGAAGACAAGAAGCACGAAAGACACCCGATC | |
| for use with | TTCGGAAACATCGTCGACGAAGTCGCATACCACGAAAAGTACCCGACAATCTACCACCTGAGAAAGAAGCTGGTCGACAGCACAGA | |
| CleanCap ™, | CAAGGCAGACCTGAGACTGATCTACCTGGCACTGGCACACATGATCAAGTTCAGAGGACACTTCCTGATCGAAGGAGACCTGAACC | |
| 5′ UTR from | CGGACAACAGCGACGTCGACAAGCTGTTCATCCAGCTGGTCCAGACATACAACCAGCTGTTCGAAGAAAACCCGATCAACGCAAGC | |
| HSD, ORF | GGAGTCGACGCAAAGGCAATCCTGAGCGCAAGACTGAGCAAGAGCAGAAGACTGGAAAACCTGATCGCACAGCTGCCGGGAGAAAA | |
| corresponding | GAAGAACGGACTGTTCGGAAACCTGATCGCACTGAGCCTGGGACTGACACCGAACTTCAAGAGCAACTTCGACCTGGCAGAAGACG | |
| to SEQ ID | CAAAGCTGCAGCTGAGCAAGGACACATACGACGACGACCTGGACAACCTGCTGGCACAGATCGGAGACCAGTACGCAGACCTGTTC | |
| NO: 4, Kozak | CTGGCAGCAAAGAACCTGAGCGACGCAATCCTGCTGAGCGACATCCTGAGAGTCAACACAGAAATCACAAAGGCACCGCTGAGCGC | |
| sequence, | AAGCATGATCAAGAGATACGACGAACACCACCAGGACCTGACACTGCTGAAGGCACTGGTCAGACAGCAGCTGCCGGAAAAGTACA | |
| and 3′ UTR | AGGAAATCTTCTTCGACCAGAGCAAGAACGGATACGCAGGATACATCGACGGAGGAGCAAGCCAGGAAGAATTCTACAAGTTCATC | |
| of ALB | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCT | |
| | AGCTAGCCATCACATTTAAAAGCATCTCAGCCTACCATGAGAATAAGAGAAAGAAAATGAAGATCAATAGCTTATTCATCTCTTTT | |
| | TCTTTTTCGTTGGTGTAAAGCCAACACCCTGTCTAAAAAACATAAATTTCTTTAATCATTTTGCCTCTTTTCTCTGTGCTTCAATT | |
| | AATAAAAAATGGAAAGAACCTCGAG | |
| 30/30/39 | AAAAAAAAAAAAAAAAAAAAAAAAAAAAAAGCGAAAAAAAAAAAAAAAAAAAAAAAAAAAAAACCGAAAAAAAAAAAAAAAAAAAA | 262 |
| poly-A | AAAAAAAAAAAAAAAAAAA | |
| sequence | | |
| poly-A 100 | AAAAAAAAAAAAAAAAAAAAAAAAAAAAAAAAAAAAAAAAAAAAAAAAAAAAAAAAAAAAAAAAAAAAAAAAAAAAAAAAAAAAAA | 263 |
| sequence | AAAAAAA | |
| | Not Used | 264 |
| ORF encoding | ATGGCAGCATTCAAGCCGAACTCGATCAACTACATCCTGGGACTGGACATCGGAATCGCATCGGTCGGATGGGCAATGGTCGAAAT | 265 |
| Neisseria | CGACGAAGAAGAAAACCCGATCAGACTGATCGACCTGGGAGTCAGAGTCTTCGAAAGAGCAGAAGTCCCGAAGACAGGAGACTCGC | |
| meningitidis | TGGCAATGGCAAGAAGACTGGCAAGATCGGTCAGAAGACTGACAAGAAGAAGAGCACACAGACTGCTGAGAACAAGAAGACTGCTG | |
| Cas9 | AAGAGAGAAGGAGTCCTGCAGGCAGCAAACTTCGACGAAAACGGACTGATCAAGTCGCTGCCGAACACACCGTGGCAGCTGAGAGC | |
| | AGCAGCACTGGACAGAAAGCTGACACCGCTGGAATGGTCGGCAGTCCTGCTGCACCTGATCAAGCACAGAGGATACCTGTCGCAGA | |
| | GAAAGAACGAAGGAGAAACAGCAGACAAGGAACTGGGAGCACTGCTGAAGGGAGTCGCAGGAAACGCACACGCACTGCAGACAGGA | |
| | GACTTCAGAACACCGGCAGAACTGGCACTGAACAAGTTCGAAAAGGAATCGGGACACATCAGAAACCAGAGATCGGACTACTCGCA | |
| | CACATTCTCGAGAAAGGACCTGCAGGCAGAACTGATCCTGCTGTTCGAAAAGCAGAAGGAATTCGGAAACCCGCACGTCTCGGGAG | |
| | GACTGAAGGAAGGAATCGAAACACTGCTGATGACACAGAGACCGGCACTGTCGGGAGACGCAGTCCAGAAGATGCTGGGACACTGC | |
| | ACATTCGAACCGGCAGAACCGAAGGCAGCAAAGAACACATACACAGCAGAAAGATTCATCTGGCTGACAAAGCTGAACAACCTGAG | |
| | AATCCTGGAACAGGGATCGGAAAGACCGCTGACAGACACAGAAAGAGCAACACTGATGGACGAACCGTACAGAAAGTCGAAGCTGA | |
| | CATACGCACAGGCAAGAAAGCTGCTGGGACTGGAAGACACAGCATTCTTCAAGGGACTGAGATACGGAAAGGACAACGCAGAAGCA | |
| | TCGACACTGATGGAAATGAAGGCATACCACGCAATCTCGAGAGCACTGGAAAAGGAAGGACTGAAGGACAAGAAGTCGCCGCTGAA | |
| | CCTGTCGCCGGAACTGCAGGACGAAATCGGAACAGCATTCTCGCTGTTCAAGACAGACGAAGACATCACAGGAAGACTGAAGGACA | |
| | GAATCCAGCCGGAAATCCTGGAAGCACTGCTGAAGCACATCTCGTTCGACAAGTTCGTCCAGATCTCGCTGAAGGCACTGAGAAGA | |
| | ATCGTCCCGCTGATGGAACAGGGAAAGAGATACGACGAAGCATGCGCAGAAATCTACGGAGACCACTACGGAAAGAAGAACACAGA | |
| | AGAAAAGATCTACCTGCCGCCGATCCCGGCAGACGAAATCAGAAACCCGGTCGTCCTGAGAGCACTGTCGCAGGCAAGAAAGGTCA | |
| | TCAACGGAGTCGTCAGAAGATACGGATCGCCGGCAAGAATCCACATCGAAACAGCAAGAGAAGTCGGAAAGTCGTTCAAGGACAGA | |
| | AAGGAAATCGAAAAGAGACAGGAAGAAAACAGAAAGGACAGAGAAAAGGCAGCAGCAAAGTTCAGAGAATACTTCCCGAACTTCGT | |
| | CGGAGAACCGAAGTCGAAGGACATCCTGAAGCTGAGACTGTACGAACAGCAGCACGGAAAGTGCCTGTACTCGGGAAAGGAAATCA | |
| | ACCTGGGAAGACTGAACGAAAAGGGATACGTCGAAATCGACCACGCACTGCCGTTCTCGAGAACATGGGACGACTCGTTCAACAAC | |
| | AAGGTCCTGGTCCTGGGATCGGAAAACCAGAACAAGGGAAACCAGACACCGTACGAATACTTCAACGGAAAGGACAACTCGAGAGA | |
| | ATGGCAGGAATTCAAGGCAAGAGTCGAAACATCGAGATTCCCGAGATCGAAGAAGCAGAGAATCCTGCTGCAGAAGTTCGACGAAG | |
| | ACGGATTCAAGGAAAGAAACCTGAACGACACAAGATACGTCAACAGATTCCTGTGCCAGTTCGTCGCAGACAGAATGAGACTGACA | |
| | GGAAAGGGAAAGAAGAGAGTCTTCGCATCGAACGGACAGATCACAAACCTGCTGAGAGGATTCTGGGGACTGAGAAAGGTCAGAGC | |
| | AGAAAACGACAGACACCACGCACTGGACGCAGTCGTCGTCGCATGCTCGACAGTCGCAATGCAGCAGAAGATCACAAGATTCGTCA | |
| | GATACAAGGAAATGAACGCATTCGACGGAAAGACAATCGACAAGGAAACAGGAGAAGTCCTGCACCAGAAGACACACTTCCCGCAG | |
| | CCGTGGGAATTCTTCGCACAGGAAGTCATGATCAGAGTCTTCGGAAAGCCGGACGGAAAGCCGGAATTCGAAGAAGCAGACACACT | |
| | GGAAAAGCTGAGAACACTGCTGGCAGAAAAGCTGTCGTCGAGACCGGAAGCAGTCCACGAATACGTCACACCGCTGTTCGTCTCGA | |
| | GAGCACCGAACAGAAAGATGTCGGGACAGGGACACATGGAAACAGTCAAGTCGGCAAAGAGACTGGACGAAGGAGTCTCGGTCCTG | |
| | AGAGTCCCGCTGACACAGCTGAAGCTGAAGGACCTGGAAAAGATGGTCAACAGAGAAAGAGAACCGAAGCTGTACGAAGCACTGAA | |
| | GGCAAGACTGGAAGCACACAAGGACGACCCGGCAAAGGCATTCGCAGAACCGTTCTACAAGTACGACAAGGCAGGAAACAGAACAC | |
| | AGCAGGTCAAGGCAGTCAGAGTCGAACAGGTCCAGAAGACAGGAGTCTGGGTCAGAAACCACAACGGAATCGCAGACAACGCAACA | |
| | ATGGTCAGAGTAGACGTCTTCGAAAAGGGAGACAAGTACTACCTGGTCCCGATCTACTCGTGGCAGGTCGCAAAGGGAATCCTGCC | |
| | GGACAGAGCAGTCGTCCAGGGAAAGGACGAAGAAGACTGGCAGCTGATCGACGACTCGTTCAACTTCAAGTTCTCGCTGCACCCGA | |
| | ACGACCTGGTCGAAGTCATCACAAAGAAGGCAAGAATGTTCGGATACTTCGCATCGTGCCACAGAGGAACAGGAAACATCAACATC | |
| | AGAATCCACGACCTGGACCACAAGATCGGAAAGAACGGAATCCTGGAAGGAATCGGAGTCAAGACAGCACTGTCGTTCCAGAAGTA | |
| | CCAGATCGACGAACTGGGAAAGGAAATCAGACCGTGCAGACTGAAGAAGAGACCGCCGGTCAGATCCGGAAAGAGAACAGCAGACG | |
| | GATCGGAATTCGAATCGCCGAAGAAGAAGAGAAAGGTCGAATGA | |
| ORF encoding | GCAGCATTCAAGCCGAACTCGATCAACTACATCCTGGGACTGGACATCGGAATCGCATCGGTCGGATGGGCAATGGTCGAAATCGA | 266 |
| Neisseria | CGAAGAAGAAAACCCGATCAGACTGATCGACCTGGGAGTCAGAGTCTTCGAAAGAGCAGAAGTCCCGAAGACAGGAGACTCGCTGG | |
| meningitidis | CAATGGCAAGAAGACTGGCAAGATCGGTCAGAAGACTGACAAGAAGAAGAGCACACAGACTGCTGAGAACAAGAAGACTGCTGAAG | |
| Cas9 (no | AGAGAAGGAGTCCTGCAGGCAGCAAACTTCGACGAAAACGGACTGATCAAGTCGCTGCCGAACACACCGTGGCAGCTGAGAGCAGC | |
| start or | AGCACTGGACAGAAAGCTGACACCGCTGGAATGGTCGGCAGTCCTGCTGCACCTGATCAAGCACAGAGGATACCTGTCGCAGAGAA | |
| stop codons; | AGAACGAAGGAGAAACAGCAGACAAGGAACTGGGAGCACTGCTGAAGGGAGTCGCAGGAAACGCACACGCACTGCAGACAGGAGAC | |
| suitable for | TTCAGAACACCGGCAGAACTGGCACTGAACAAGTTCGAAAAGGAATCGGGACACATCAGAAACCAGAGATCGGACTACTCGCACAC | |
| inclusion in | ATTCTCGAGAAAGGACCTGCAGGCAGAACTGATCCTGCTGTTCGAAAAGCAGAAGGAATTCGGAAACCCGCACGTCTCGGGAGGAC | |
| fusion | TGAAGGAAGGAATCGAAACACTGCTGATGACACAGAGACCGGCACTGTCGGGAGACGCAGTCCAGAAGATGCTGGGACACTGCACA | |
| protein | TTCGAACCGGCAGAACCGAAGGCAGCAAAGAACACATACACAGCAGAAAGATTCATCTGGCTGACAAAGCTGAACAACCTGAGAAT | |
| coding | CCTGGAACAGGGATCGGAAAGACCGCTGACAGACACAGAAAGAGCAACACTGATGGACGAACCGTACAGAAAGTCGAAGCTGACAT | |
| sequence) | ACGCACAGGCAAGAAAGCTGCTGGGACTGGAAGACACAGCATTCTTCAAGGGACTGAGATACGGAAAGGACAACGCAGAAGCATCG | |
| | ACACTGATGGAAATGAAGGCATACCACGCAATCTCGAGAGCACTGGAAAAGGAAGGACTGAAGGACAAGAAGTCGCCGCTGAACCT | |
| | GTCGCCGGAACTGCAGGACGAAATCGGAACAGCATTCTCGCTGTTCAAGACAGACGAAGACATCACAGGAAGACTGAAGGACAGAA | |
| | TCCAGCCGGAAATCCTGGAAGCACTGCTGAAGCACATCTCGTTCGACAAGTTCGTCCAGATCTCGCTGAAGGCACTGAGAAGAATC | |
| | GTCCCGCTGATGGAACAGGGAAAGAGATACGACGAAGCATGCGCAGAAATCTACGGAGACCACTACGGAAAGAAGAACACAGAAGA | |
| | AAAGATCTACCTGCCGCCGATCCCGGCAGACGAAATCAGAAACCCGGTCGTCCTGAGAGCACTGTCGCAGGCAAGAAAGGTCATCA | |
| | ACGGAGTCGTCAGAAGATACGGATCGCCGGCAAGAATCCACATCGAAACAGCAAGAGAAGTCGGAAAGTCGTTCAAGGACAGAAAG | |
| | GAAATCGAAAAGAGACAGGAAGAAAACAGAAAGGACAGAGAAAAGGCAGCAGCAAAGTTCAGAGAATACTTCCCGAACTTCGTCGG | |
| | AGAACCGAAGTCGAAGGACATCCTGAAGCTGAGACTGTACGAACAGCAGCACGGAAAGTGCCTGTACTCGGGAAAGGAAATCAACC | |
| | TGGGAAGACTGAACGAAAAGGGATACGTCGAAATCGACCACGCACTGCCGTTCTCGAGAACATGGGACGACTCGTTCAACAACAAG | |
| | GTCCTGGTCCTGGGATCGGAAAACCAGAACAAGGGAAACCAGACACCGTACGAATACTTCAACGGAAAGGACAACTCGAGAGAATG | |
| | GCAGGAATTCAAGGCAAGAGTCGAAACATCGAGATTCCCGAGATCGAAGAAGCAGAGAATCCTGCTGCAGAAGTTCGACGAAGACG | |
| | GATTCAAGGAAAGAAACCTGAACGACACAAGATACGTCAACAGATTCCTGTGCCAGTTCGTCGCAGACAGAATGAGACTGACAGGA | |
| | AAGGGAAAGAAGAGAGTCTTCGCATCGAACGGACAGATCACAAACCTGCTGAGAGGATTCTGGGGACTGAGAAAGGTCAGAGCAGA | |
| | AAACGACAGACACCACGCACTGGACGCAGTCGTCGTCGCATGCTCGACAGTCGCAATGCAGCAGAAGATCACAAGATTCGTCAGAT | |
| | ACAAGGAAATGAACGCATTCGACGGAAAGACAATCGACAAGGAAACAGGAGAAGTCCTGCACCAGAAGACACACTTCCCGCAGCCG | |
| | TGGGAATTCTTCGCACAGGAAGTCATGATCAGAGTCTTCGGAAAGCCGGACGGAAAGCCGGAATTCGAAGAAGCAGACACACTGGA | |
| | AAAGCTGAGAACACTGCTGGCAGAAAAGCTGTCGTCGAGACCGGAAGCAGTCCACGAATACGTCACACCGCTGTTCGTCTCGAGAG | |
| | CACCGAACAGAAAGATGTCGGGACAGGGACACATGGAAACAGTCAAGTCGGCAAAGAGACTGGACGAAGGAGTCTCGGTCCTGAGA | |
| | GTCCCGCTGACACAGCTGAAGCTGAAGGACCTGGAAAAGATGGTCAACAGAGAAAGAGAACCGAAGCTGTACGAAGCACTGAAGGC | |
| | AAGACTGGAAGCACACAAGGACGACCCGGCAAAGGCATTCGCAGAACCGTTCTACAAGTACGACAAGGCAGGAAACAGAACACAGC | |
| | AGGTCAAGGCAGTCAGAGTCGAACAGGTCCAGAAGACAGGAGTCTGGGTCAGAAACCACAACGGAATCGCAGACAACGCAACAATG | |
| | GTCAGAGTAGACGTCTTCGAAAAGGGAGACAAGTACTACCTGGTCCCGATCTACTCGTGGCAGGTCGCAAAGGGAATCCTGCCGGA | |
| | CAGAGCAGTCGTCCAGGGAAAGGACGAAGAAGACTGGCAGCTGATCGACGACTCGTTCAACTTCAAGTTCTCGCTGCACCCGAACG | |
| | ACCTGGTCGAAGTCATCACAAAGAAGGCAAGAATGTTCGGATACTTCGCATCGTGCCACAGAGGAACAGGAAACATCAACATCAGA | |
| | ATCCACGACCTGGACCACAAGATCGGAAAGAACGGAATCCTGGAAGGAATCGGAGTCAAGACAGCACTGTCGTTCCAGAAGTACCA | |
| | GATCGACGAACTGGGAAAGGAAATCAGACCGTGCAGACTGAAGAAGAGACCGCCGGTCAGATCCGGAAAGAGAACAGCAGACGGAT | |
| | CGGAATTCGAATCGCCGAAGAAGAAGAGAAAGGTCGAA | |
| Transcript | GGGAGACCCAAGCTGGCTAGCGTTTAAACTTAAGCTTGGATCCGCCACCATGGCAGCATTCAAGCCGAACTCGATCAACTACATCC | 267 |
| comprising | TGGGACTGGACATCGGAATCGCATCGGTCGGATGGGCAATGGTCGAAATCGACGAAGAAGAAAACCCGATCAGACTGATCGACCTG | |
| SEQ ID NO: | GGAGTCAGAGTCTTCGAAAGAGCAGAAGTCCCGAAGACAGGAGACTCGCTGGCAATGGCAAGAAGACTGGCAAGATCGGTCAGAAG | |
| 65 (encoding | ACTGACAAGAAGAAGAGCACACAGACTGCTGAGAACAAGAAGACTGCTGAAGAGAGAAGGAGTCCTGCAGGCAGCAAACTTCGACG | |
| Neisseria | AAAACGGACTGATCAAGTCGCTGCCGAACACACCGTGGCAGCTGAGAGCAGCAGCACTGGACAGAAAGCTGACACCGCTGGAATGG | |
| meningitidis | TCGGCAGTCCTGCTGCACCTGATCAAGCACAGAGGATACCTGTCGCAGAGAAAGAACGAAGGAGAAACAGCAGACAAGGAACTGGG | |
| Cas9) | AGCACTGCTGAAGGGAGTCGCAGGAAACGCACACGCACTGCAGACAGGAGACTTCAGAACACCGGCAGAACTGGCACTGAACAAGT | |
| | TCGAAAAGGAATCGGGACACATCAGAAACCAGAGATCGGACTACTCGCACACATTCTCGAGAAAGGACCTGCAGGCAGAACTGATC | |
| | CTGCTGTTCGAAAAGCAGAAGGAATTCGGAAACCCGCACGTCTCGGGAGGACTGAAGGAAGGAATCGAAACACTGCTGATGACACA | |
| | GAGACCGGCACTGTCGGGAGACGCAGTCCAGAAGATGCTGGGACACTGCACATTCGAACCGGCAGAACCGAAGGCAGCAAAGAACA | |
| | CATACACAGCAGAAAGATTCATCTGGCTGACAAAGCTGAACAACCTGAGAATCCTGGAACAGGGATCGGAAAGACCGCTGACAGAC | |
| | ACAGAAAGAGCAACACTGATGGACGAACCGTACAGAAAGTCGAAGCTGACATACGCACAGGCAAGAAAGCTGCTGGGACTGGAAGA | |
| | CACAGCATTCTTCAAGGGACTGAGATACGGAAAGGACAACGCAGAAGCATCGACACTGATGGAAATGAAGGCATACCACGCAATCT | |
| | CGAGAGCACTGGAAAAGGAAGGACTGAAGGACAAGAAGTCGCCGCTGAACCTGTCGCCGGAACTGCAGGACGAAATCGGAACAGCA | |
| | TTCTCGCTGTTCAAGACAGACGAAGACATCACAGGAAGACTGAAGGACAGAATCCAGCCGGAAATCCTGGAAGCACTGCTGAAGCA | |
| | CATCTCGTTCGACAAGTTCGTCCAGATCTCGCTGAAGGCACTGAGAAGAATCGTCCCGCTGATGGAACAGGGAAAGAGATACGACG | |
| | AAGCATGCGCAGAAATCTACGGAGACCACTACGGAAAGAAGAACACAGAAGAAAAGATCTACCTGCCGCCGATCCCGGCAGACGAA | |
| | ATCAGAAACCCGGTCGTCCTGAGAGCACTGTCGCAGGCAAGAAAGGTCATCAACGGAGTCGTCAGAAGATACGGATCGCCGGCAAG | |
| | AATCCACATCGAAACAGCAAGAGAAGTCGGAAAGTCGTTCAAGGACAGAAAGGAAATCGAAAAGAGACAGGAAGAAAACAGAAAGG | |
| | ACAGAGAAAAGGCAGCAGCAAAGTTCAGAGAATACTTCCCGAACTTCGTCGGAGAACCGAAGTCGAAGGACATCCTGAAGCTGAGA | |
| | CTGTACGAACAGCAGCACGGAAAGTGCCTGTACTCGGGAAAGGAAATCAACCTGGGAAGACTGAACGAAAAGGGATACGTCGAAAT | |
| | CGACCACGCACTGCCGTTCTCGAGAACATGGGACGACTCGTTCAACAACAAGGTCCTGGTCCTGGGATCGGAAAACCAGAACAAGG | |
| | GAAACCAGACACCGTACGAATACTTCAACGGAAAGGACAACTCGAGAGAATGGCAGGAATTCAAGGCAAGAGTCGAAACATCGAGA | |
| | TTCCCGAGATCGAAGAAGCAGAGAATCCTGCTGCAGAAGTTCGACGAAGACGGATTCAAGGAAAGAAACCTGAACGACACAAGATA | |
| | CGTCAACAGATTCCTGTGCCAGTTCGTCGCAGACAGAATGAGACTGACAGGAAAGGGAAAGAAGAGAGTCTTCGCATCGAACGGAC | |
| | AGATCACAAACCTGCTGAGAGGATTCTGGGGACTGAGAAAGGTCAGAGCAGAAAACGACAGACACCACGCACTGGACGCAGTCGTC | |
| | GTCGCATGCTCGACAGTCGCAATGCAGCAGAAGATCACAAGATTCGTCAGATACAAGGAAATGAACGCATTCGACGGAAAGACAAT | |
| | CGACAAGGAAACAGGAGAAGTCCTGCACCAGAAGACACACTTCCCGCAGCCGTGGGAATTCTTCGCACAGGAAGTCATGATCAGAG | |
| | TCTTCGGAAAGCCGGACGGAAAGCCGGAATTCGAAGAAGCAGACACACTGGAAAAGCTGAGAACACTGCTGGCAGAAAAGCTGTCG | |
| | TCGAGACCGGAAGCAGTCCACGAATACGTCACACCGCTGTTCGTCTCGAGAGCACCGAACAGAAAGATGTCGGGACAGGGACACAT | |
| | GGAAACAGTCAAGTCGGCAAAGAGACTGGACGAAGGAGTCTCGGTCCTGAGAGTCCCGCTGACACAGCTGAAGCTGAAGGACCTGG | |
| | AAAAGATGGTCAACAGAGAAAGAGAACCGAAGCTGTACGAAGCACTGAAGGCAAGACTGGAAGCACACAAGGACGACCCGGCAAAG | |
| | GCATTCGCAGAACCGTTCTACAAGTACGACAAGGCAGGAAACAGAACACAGCAGGTCAAGGCAGTCAGAGTCGAACAGGTCCAGAA | |
| | GACAGGAGTCTGGGTCAGAAACCACAACGGAATCGCAGACAACGCAACAATGGTCAGAGTAGACGTCTTCGAAAAGGGAGACAAGT | |
| | ACTACCTGGTCCCGATCTACTCGTGGCAGGTCGCAAAGGGAATCCTGCCGGACAGAGCAGTCGTCCAGGGAAAGGACGAAGAAGAC | |
| | TGGCAGCTGATCGACGACTCGTTCAACTTCAAGTTCTCGCTGCACCCGAACGACCTGGTCGAAGTCATCACAAAGAAGGCAAGAAT | |
| | GTTCGGATACTTCGCATCGTGCCACAGAGGAAaAGGAAACATCAACATCAGAATCCACGACCTGGACCACAAGATCGGAAAGAACG | |
| | GAATCCTGGAAGGAATCGGAGTCAAGACAGCACTGTCGTTCCAGAAGTACCAGATCGACGAACTGGGAAAGGAAATCAGACCGTGC | |
| | AGACTGAAGAAGAGACCGCCGGTCAGATCCGGAAAGAGAACAGCAGACGGATCGGAATTCGAATCGCCGAAGAAGAAGAGAAAGGT | |
| | CGAATGATAGCTAGCTCGAGTCTAGAGGGCCCGTTTAAACCCGCTGATCAGCCTCGACTGTGCCTTCTAGTTGCCAGCCATCTGTT | |
| | GTTTGCCCCTCCCCCGTGCCTTCCTTGACCCTGGAAGGTGCCACTCCCACTGTCCTTTCCTAATAAAATGAGGAAATTGCATCGCA | |
| | TTGTCTGAGTAGGTGTCATTCTATTCTGGGGGGTGGGGTGGGGCAGGACAGCAAGGGGGAGGATTGGGAAGACAATAGCAGGCATG | |
| | CTGGGGATGCGGTGGGCTCTATGG | |
| Amino acid | MAAFKPNSINYILGLDIGIASVGWAMVEIDEEENPIRLIDLGVRVFERAEVPKTGDSLAMARRLARSVRRLTRRRAHRLLRTRRLL | 268 |
| sequence of | KREGVLQAANFDENGLIKSLPNTPWQLRAAALDRKLTPLEWSAVLLHLIKHRGYLSQRKNEGETADKELGALLKGVAGNAHALQTG | |
| Neisseria | DFRTPAELALNKFEKESGHIRNQRSDYSHTFSRKDLQAELILLFEKQKEFGNPHVSGGLKEGIETLLMTQRPALSGDAVQKMLGHC | |
| meningitidis | TFEPAEPKAAKNTYTAERFIWLTKLNNLRILEQGSERPLTDTERATLMDEPYRKSKLTYAQARKLLGLEDTAFFKGLRYGKDNAEA | |
| Cas9 | STLMEMKAYHAISRALEKEGLKDKKSPLNLSPELQDEIGTAFSLFKTDEDITGRLKDRIQPEILEALLKHISFDKFVQISLKALRR | |
| | IVPLMEQGKRYDEACAEIYGDHYGKKNTEEKIYLPPIPADEIRNPVVLRALSQARKVINGVVRRYGSPARIHIETAREVGKSFKDR | |
| | KEIEKRQEENRKDREKAAAKFREYFPNFVGEPKSKDILKLRLYEQQHGKCLYSGKEINLGRLNEKGYVEIDHALPFSRTWDDSFNN | |
| | KVLVLGSENQNKGNQTPYEYFNGKDNSREWQEFKARVETSRFPRSKKQRILLQKFDEDGFKERNLNDTRYVNRFLCQFVADRMRLT | |
| | GKGKKRVFASNGQITNLLRGFWGLRKVRAENDRHHALDAVVVACSTVAMQQKITRFVRYKEMNAFDGKTIDKETGEVLHQKTHFPQ | |
| | PWEFFAQEVMIRVFGKPDGKPEFEEADTLEKLRTLLAEKLSSRPEAVHEYVTPLFVSRAPNRKMSGQGHMETVKSAKRLDEGVSVL | |
| | RVPLTQLKLKDLEKMVNREREPKLYEALKARLEAHKDDPAKAFAEPFYKYDKAGNRTQQVKAVRVEQVQKTGVWVRNHNGIADNAT | |
| | MVRVDVFEKGDKYYLVPIYSWQVAKGILPDRAVVQGKDEEDWQLIDDSFNFKFSLHPNDLVEVITKKARMFGYFASCHRGTGNINI | |
| | RIHDLDHKIGKNGILEGIGVKTALSFQKYQIDELGKEIRPCRLKKRPPVRSGKRTADGSEFESPKKKRKVE | |
| | Not Used | 269 |
| G502 guide | mA*mC*mA*CAAAUACCAGUCCAGCGGUUUUAGAmGmCmUmAmGmAmAmAmUmAmGmCAAGUUAAAAUAAGGCUAGUCCGUUAUCA | 270 |
| RNA | mAmCmUmUmGGmUmGmGmCmAmCmCmGmAmGmUmCmGmGmUmGmCmU*mU*mU*mU | |
| G509 guide | mA*mA*mA*GUUCUAGAUGCCGUCCGGUUUUAGAmGmCmUmAmGmAmAmAmUmAmGmCAAGUUAAAAUAAGGCUAGUCCGUUAUCA | 271 |
| RNA | mAmCmUmUmGGmUmGmGmCmAmCmCmGmAmGmUmCmGmGmUmGmCmU*mU*mU*mU | |
| G534 guide | mA*mC*mG*CAAAUAUCAGUCCAGCGGUUUUAGAmGmCmUmAmGmAmAmAmUmAmGmCAAGUUAAAAUAAGGCUAGUCCGUUAUCA | 272 |
| RNA | mAmCmUmUmGGmUmGmGmCmAmCmCmGmAmGmUmCmGmGmUmGmCmU*mU*mU*mU | |
| DNA coding | TCGCGCGTTTCGGTGATGACGGTGAAAACCTCTGACACATGCAGCTCCCGGAGACGGTCACAGCTTGTCTGTAAGCGGATGCCGGG | 273 |
| sequence of | AGCAGACAAGCCCGTCAGGGCGCGTCAGCGGGTGTTGGCGGGTGTCGGGGCTGGCTTAACTATGCGGCATCAGAGCAGATTGTACT | |
| eGFP | GAGAGTGCACCATATGCGGTGTGAAATACCGCACAGATGCGTAAGGAGAAAATACCGCATCAGGCGCCATTCGCCATTCAGGCTGC | |
| | GCAACTGTTGGGAAGGGCGATCGGTGCGGGCCTCTTCGCTATTACGCCAGCTGGCGAAAGGGGGATGTGCTGCAAGGCGATTAAGT | |
| | TGGGTAACGCCAGGGTTTTCCCAGTCACGACGTTGTAAAACGACGGCCAGTGAATTCTAATACGACTCACTATAGGGTCCCGCAGT | |
| | CGGCGTCCAGCGGCTCTGCTTGTTCGTGTGTGTGTCGTTGCAGGCCTTATTCGGATCCATGGTGAGCAAGGGCGAGGAGCTGTTCA | |
| | CCGGGGTGGTGCCCATCCTGGTCGAGCTGGACGGCGACGTAAACGGCCACAAGTTCAGCGTGTCCGGCGAGGGCGAGGGCGATGCC | |
| | ACCTACGGCAAGCTGACCCTGAAGTTCATCTGCACCACCGGCAAGCTGCCCGTGCCCTGGCCCACCCTCGTGACCACCCTGACCTA | |
| | CGGCGTGCAGTGCTTCAGCCGCTACCCCGACCACATGAAGCAGCACGACTTCTTCAAGTCCGCCATGCCCGAAGGCTACGTCCAGG | |
| | AGCGCACCATCTTCTTCAAGGACGACGGCAACTACAAGACCCGCGCCGAGGTGAAGTTCGAGGGCGACACCCTGGTGAACCGCATC | |
| | GAGCTGAAGGGCATCGACTTCAAGGAGGACGGCAACATCCTGGGGCACAAGCTGGAGTACAACTACAACAGCCACAACGTCTATAT | |
| | CATGGCCGACAAGCAGAAGAACGGCATCAAGGTGAACTTCAAGATCCGCCACAACATCGAGGACGGCAGCGTGCAGCTCGCCGACC | |
| | ACTACCAGCAGAACACCCCCATCGGCGACGGCCCCGTGCTGCTGCCCGACAACCACTACCTGAGCACCCAGTCCGCCCTGAGCAAA | |
| | GACCCCAACGAGAAGCGCGATCACATGGTCCTGCTGGAGTTCGTGACCGCCGCCGGGATCACTCTCGGCATGGACGAGCTGTACAA | |
| | GTAATAGGAATTATGCAGTCTAGCCATCACATTTAAAAGCATCTCAGCCTACCATGAGAATAAGAGAAAGAAAATGAAGATCAATA | |
| | GCTTATTCATCTCTTTTTCTTTTTCGTTGGTGTAAAGCCAACACCCTGTCTAAAAAACATAAATTTCTTTAATCATTTTGCCTCTT | |
| | TTCTCTGTGCTTCAATTAATAAAAAATGGAAAGAACCTCGAGAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAATCTAGACTTAAGCTTGATGAGCTCTAGCTT | |
| | GGCGTAATCATGGTCATAGCTGTTTCCTGTGTGAAATTGTTATCCGCTCACAATTCCACACAACATACGAGCCGGAAGCATAAAGT | |
| | GTAAAGCCTGGGGTGCCTAATGAGTGAGCTAACTCACATTAATTGCGTTGCGCTCACTGCCCGCTTTCCAGTCGGGAAACCTGTCG | |
| | TGCCAGCTGCATTAATGAATCGGCCAACGCGCGGGGAGAGGCGGTTTGCGTATTGGGCGCTCTTCCGCTTCCTCGCTCACTGACTC | |
| | GCTGCGCTCGGTCGTTCGGCTGCGGCGAGCGGTATCAGCTCACTCAAAGGCGGTAATACGGTTATCCACAGAATCAGGGGATAACG | |
| | CAGGAAAGAACATGTGAGCAAAAGGCCAGCAAAAGGCCAGGAACCGTAAAAAGGCCGCGTTGCTGGCGTTTTTCCATAGGCTCCGC | |
| | CCCCCTGACGAGCATCACAAAAATCGACGCTCAAGTCAGAGGTGGCGAAACCCGACAGGACTATAAAGATACCAGGCGTTTCCCCC | |
| | TGGAAGCTCCCTCGTGCGCTCTCCTGTTCCGACCCTGCCGCTTACCGGATACCTGTCCGCCTTTCTCCCTTCGGGAAGCGTGGCGC | |
| | TTTCTCATAGCTCACGCTGTAGGTATCTCAGTTCGGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAGACACGACTTATCGCCACTGGCAGCAGCCACTGG | |
| | TAACAGGATTAGCAGAGCGAGGTATGTAGGCGGTGCTACAGAGTTCTTGAAGTGGTGGCCTAACTACGGCTACACTAGAAGAACAG | |
| | TATTTGGTATCTGCGCTCTGCTGAAGCCAGTTACCTTCGGAAAAAGAGTTGGTAGCTCTTGATCCGGCAAACAAACCACCGCTGGT | |
| | AGCGGTGGTTTTTTTGTTTGCAAGCAGCAGATTACGCGCAGAAAAAAAGGATCTCAAGAAGATCCTTTGATCTTTTCTACGGGGTC | |
| | TGACGCTCAGTGGAACGAAAACTCACGTTAAGGGATTTTGGTCATGAGATTATCAAAAAGGATCTTCACCTAGATCCTTTTAAATT | |
| | AAAAATGAAGTTTTAAATCAATCTAAAGTATATATGAGTAAACTTGGTCTGACAGTTACCAATGCTTAATCAGTGAGGCACCTATC | |
| | TCAGCGATCTGTCTATTTCGTTCATCCATAGTTGCCTGACTCCCCGTCGTGTAGATAACTACGATACGGGAGGGCTTACCATCTGG | |
| | CCCCAGTGCTGCAATGATACCGCGAGACCCACGCTCACCGGCTCCAGATTTATCAGCAATAAACCAGCCAGCCGGAAGGGCCGAGC | |
| | GCAGAAGTGGTCCTGCAACTTTATCCGCCTCCATCCAGTCTATTAATTGTTGCCGGGAAGCTAGAGTAAGTAGTTCGCCAGTTAAT | |
| | AGTTTGCGCAACGTTGTTGCCATTGCTACAGGCATCGTGGTGTCACGCTCGTCGTTTGGTATGGCTTCATTCAGCTCCGGTTCCCA | |
| | ACGATCAAGGCGAGTTACATGATCCCCCATGTTGTGCAAAAAAGCGGTTAGCTCCTTCGGTCCTCCGATCGTTGTCAGAAGTAAGT | |
| | TGGCCGCAGTGTTATCACTCATGGTTATGGCAGCACTGCATAATTCTCTTACTGTCATGCCATCCGTAAGATGCTTTTCTGTGACT | |
| | GGTGAGTACTCAACCAAGTCATTCTGAGAATAGTGTATGCGGCGACCGAGTTGCTCTTGCCCGGCGTCAATACGGGATAATACCGC | |
| | GCCACATAGCAGAACTTTAAAAGTGCTCATCATTGGAAAACGTTCTTCGGGGCGAAAACTCTCAAGGATCTTACCGCTGTTGAGAT | |
| | CCAGTTCGATGTAACCCACTCGTGCACCCAACTGATCTTCAGCATCTTTTACTTTCACCAGCGTTTCTGGGTGAGCAAAAACAGGA | |
| | AGGCAAAATGCCGCAAAAAAGGGAATAAGGGCGACACGGAAATGTTGAATACTCATACTCTTCCTTTTTCAATATTATTGAAGCAT | |
| | TTATCAGGGTTATTGTCTCATGAGCGGATACATATTTGAATGTATTTAGAAAAATAAACAAATAGGGGTTCCGCGCACATTTCCCC | |
| | GAAAAGTGCCACCTGACGTCTAAGAAACCATTATTATCATGACATTAACCTATAAAAATAGGCGTATCACGAGGCCCTTTCGTCG | |
| | Not Used | 274 |
| CMV-1 5′ UTR | CAGATCGCCTGGAGACGCCATCCACGCTGTTTTGACCTCCAT | 275 |
| CNV-2 5′ UTR | AGAAGACACCGGGACCGATCCAGCCTCCGCGGCCGGGAACGG | 276 |
| CNV-3 5′ UTR | TGCATTGGAACGCGGATTCCCCGTGCCAAGAGTGACTCACCG | 277 |
| SV40 NLS | PKKKRKV | 278 |
| Exemplary | LAAKRSRTT | 279 |
| NLS 1 | | |
| Exemplary | QAAKRSRTT | 280 |
| NLS 2 | | |
| Exemplary | PAPAKRERTT | 281 |
| NLS 3 | | |
| Exemplary | QAAKRPRTT | 282 |
| NLS 4 | | |
| Exemplary | RAAKRPRTT | 283 |
| NLS 5 | | |
| Exemplary | AAAKRSWSMAA | 284 |
| NLS 6 | | |
| Exemplary | AAAKRVWSMAF | 285 |
| NLS 7 | | |
| Exemplary | AAAKRSWSMAF | 286 |
| NLS 8 | | |
| Exemplary | AAAKRKYFAA | 287 |
| NLS9 | | |
| Exemplary | RAAKRKAFAA | 288 |
| NLS 10 | | |
| Exemplary | RAAKRKYFAV | 289 |
| NLS 11 | | |
| Alternate | PKKKRRV | 290 |
| SV40 NLS | | |
| Nucleoplasmin | KRPAATKKAGQAKKKK | 291 |
| NLS | | |
| Exemplary | CCGAAGAAGAAGAGAAAGGTC | 292 |
| coding | | |
| sequence for | | |
| SV40 NLS | | |
| Exemplary | CTGGCAGCAAAGAGAAGCAGAACAACA | 293 |
| coding | | |
| sequence for | | |
| NLS1 | | |
| Exemplary | CAGGCAGCAAAGAGAAGCAGAACAACA | 294 |
| coding | | |
| sequence for | | |
| NLS2 | | |
| Exemplary | CCGGCACCGGCAAAGAGAGAAAGAACAACA | 295 |
| coding | | |
| sequence for | | |
| NLS3 | | |
| Exemplary | CAGGCAGCAAAGAGACCGAGAACAACA | 296 |
| coding | | |
| sequence for | | |
| NLS4 | | |
| Exemplary | AGAGCAGCAAAGAGACCGAGAACAACA | 297 |
| coding | | |
| sequence for | | |
| NLS5 | | |
| Exemplary | GCAGCAGCAAAGAGAAGCTGGAGCATGGCAGCA | 298 |
| coding | | |
| sequence for | | |
| NLS6 | | |
| Exemplary | GCAGCAGCAAAGAGAGTCTGGAGCATGGCATTC | 299 |
| coding | | |
| sequence for | | |
| NLS7 | | |
| Exemplary | GCAGCAGCAAAGAGAAGCTGGAGCATGGCATTC | 300 |
| coding | | |
| sequence for | | |
| NLS8 | | |
| Exemplary | GCAGCAGCAAAGAGAAAGTACTTCGCAGCA | 301 |
| coding | | |
| sequence for | | |
| NLS9 | | |
| Exemplary | AGAGCAGCAAAGAGAAAGGCATTCGCAGCA | 302 |
| coding | | |
| sequence for | | |
| NLS10 | | |
| Exemplary | AGAGCAGCAAAGAGAAAGTACTTCGCAGTC | 303 |
| coding | | |
| sequence for | | |
| NLS11 | | |
| Exemplary | CCGAAGAAGAAGAGAAGAGTC | 304 |
| coding | | |
| sequence for | | |
| alternate | | |
| SV40 NLS | | |
| exemplary | gccgccRccAUGG | 305 |
| Kozak | | |
| sequence | | |
| | Not Used | 306 |
| Cas9 ORF | ATGGACAAGAAGTACTCTATCGGTTTGGACATCGGTACCAACTCTGTCGGTTGGGCCGTCATCACCGACGAATACAAGGTCCCATC | 307 |
| using long | TAAGAAGTTCAAGGTCTTGGGTAACACCGACAGACACTCTATCAAGAAGAACTTGATCGGTGCCTTGTTGTTCGACTCTGGTGAAA | |
| half life | CCGCCGAAGCCACCAGATTGAAGAGAACCGCCAGAAGAAGATACACCAGAAGAAAGAACAGAATCTGCTACTTGCAAGAAATCTTC | |
| codons of | TCTAACGAAATGGCCAAGGTCGACGACTCTTTCTTCCACAGATTGGAAGAATCTTTCTTGGTCGAAGAAGACAAGAAGCACGAAAG | |
| Table 4, | ACACCCAATCTTCGGTAACATCGTCGACGAAGTCGCCTACCACGAAAAGTACCCAACCATCTACCACTTGAGAAAGAAGTTGGTCG | |
| with start | ACTCTACCGACAAGGCCGACTTGAGATTGATCTACTTGGCCTTGGCCCACATGATCAAGTTCAGAGGTCACTTCTTGATCGAAGGT | |
| and stop | GACTTGAACCCAGACAACTCTGACGTCGACAAGTTGTTCATCCAATTGGTCCAAACCTACAACCAATTGTTCGAAGAAAACCCAAT | |
| codons | CAACGCCTCTGGTGTCGACGCCAAGGCCATCTTGTCTGCCAGATTGTCTAAGAGCAGAAGATTGGAAAACTTGATCGCCCAATTGC | |
| | CAGGTGAAAAGAAGAACGGTTTGTTCGGTAACTTGATCGCCTTGTCTTTGGGTTTGACCCCAAACTTCAAGTCTAACTTCGACTTG | |
| | GCCGAAGACGCCAAGTTGCAATTGTCTAAGGACACCTACGACGACGACTTGGACAACTTGTTGGCCCAAATCGGTGACCAATACGC | |
| | CGACTTGTTCTTGGCCGCCAAGAACTTGTCTGACGCCATCTTGTTGTCTGACATCTTGAGAGTCAACACCGAAATCACCAAGGCCC | |
| | CATTGTCTGCCTCTATGATCAAGAGATACGACGAACACCACCAAGACTTGACCTTGTTGAAGGCCTTGGTCAGACAACAATTGCCA | |
| | GAAAAGTACAAGGAAATCTTCTTCGACCAATCTAAGAACGGTTACGCCGGTTACATCGACGGTGGTGCCTCTCAAGAAGAATTCTA | |
| | CAAGTTCATCAAGCCAATCTTGGAAAAGATGGACGGTACCGAAGAATTGTTGGTCAAGTTGAACAGAGAAGACTTGTTGAGAAAGC | |
| | AAAGAACCTTCGACAACGGTTCTATCCCACACCAAATCCACTTGGGTGAATTGCACGCCATCTTGAGAAGACAAGAAGACTTCTAC | |
| | CCATTCTTGAAGGACAACAGAGAAAAGATCGAAAAGATCTTGACCTTCAGAATCCCATACTACGTCGGTCCATTGGCCAGAGGTAA | |
| | CAGCAGATTCGCCTGGATGACCAGAAAGTCTGAAGAAACCATCACCCCATGGAACTTCGAAGAAGTCGTCGACAAGGGTGCCTCTG | |
| | CCCAATCTTTCATCGAAAGAATGACCAACTTCGACAAGAACTTGCCAAACGAAAAGGTCTTGCCAAAGCACTCTTTGTTGTACGAA | |
| | TACTTCACCGTCTACAACGAATTGACCAAGGTCAAGTACGTCACCGAAGGTATGAGAAAGCCAGCCTTCTTGTCTGGTGAACAAAA | |
| | GAAGGCCATCGTCGACTTGTTGTTCAAGACCAACAGAAAGGTCACCGTCAAGCAATTGAAGGAAGACTACTTCAAGAAGATCGAAT | |
| | GCTTCGACTCTGTCGAAATCTCTGGTGTCGAAGACAGATTCAACGCCTCTTTGGGTACCTACCACGACTTGTTGAAGATCATCAAG | |
| | GACAAGGACTTCTTGGACAACGAAGAAAACGAAGACATCTTGGAAGACATCGTCTTGACCTTGACCTTGTTCGAAGACAGAGAAAT | |
| | GATCGAAGAAAGATTGAAGACCTACGCCCACTTGTTCGACGACAAGGTCATGAAGCAATTGAAGAGAAGAAGATACACCGGTTGGG | |
| | GTAGATTGAGCAGAAAGTTGATCAACGGTATCAGAGACAAGCAATCTGGTAAGACCATCTTGGACTTCTTGAAGTCTGACGGTTTC | |
| | GCCAACAGAAACTTCATGCAATTGATCCACGACGACTCTTTGACCTTCAAGGAAGACATCCAAAAGGCCCAAGTCTCTGGTCAAGG | |
| | TGACTCTTTGCACGAACACATCGCCAACTTGGCCGGTTCTCCAGCCATCAAGAAGGGTATCTTGCAAACCGTCAAGGTCGTCGACG | |
| | AATTGGTCAAGGTCATGGGTAGACACAAGCCAGAAAACATCGTCATCGAAATGGCCAGAGAAAACCAAACCACCCAAAAGGGTCAA | |
| | AAGAACAGCAGAGAAAGAATGAAGAGAATCGAAGAAGGTATCAAGGAATTGGGTTCTCAAATCTTGAAGGAACACCCAGTCGAAAA | |
| | CACCCAATTGCAAAACGAAAAGTTGTACTTGTACTACTTGCAAAACGGTAGAGACATGTACGTCGACCAAGAATTGGACATCAACA | |
| | GATTGTCTGACTACGACGTCGACCACATCGTCCCACAATCTTTCTTGAAGGACGACTCTATCGACAACAAGGTCTTGACCAGATCT | |
| | GACAAGAACAGAGGTAAGTCTGACAACGTCCCATCTGAAGAAGTCGTCAAGAAGATGAAGAACTACTGGAGACAATTGTTGAACGC | |
| | CAAGTTGATCACCCAAAGAAAGTTCGACAACTTGACCAAGGCCGAAAGAGGTGGTTTGTCTGAATTGGACAAGGCCGGTTTCATCA | |
| | AGAGACAATTGGTCGAAACCAGACAAATCACCAAGCACGTCGCCCAAATCTTGGACAGCAGAATGAACACCAAGTACGACGAAAAC | |
| | GACAAGTTGATCAGAGAAGTCAAGGTCATCACCTTGAAGTCTAAGTTGGTCTCTGACTTCAGAAAGGACTTCCAATTCTACAAGGT | |
| | CAGAGAAATCAACAACTACCACCACGCCCACGACGCCTACTTGAACGCCGTCGTCGGTACCGCCTTGATCAAGAAGTACCCAAAGT | |
| | TGGAATCTGAATTCGTCTACGGTGACTACAAGGTCTACGACGTCAGAAAGATGATCGCCAAGTCTGAACAAGAAATCGGTAAGGCC | |
| | ACCGCCAAGTACTTCTTCTACTCTAACATCATGAACTTCTTCAAGACCGAAATCACCTTGGCCAACGGTGAAATCAGAAAGAGACC | |
| | ATTGATCGAAACCAACGGTGAAACCGGTGAAATCGTCTGGGACAAGGGTAGAGACTTCGCCACCGTCAGAAAGGTCTTGTCTATGC | |
| | CACAAGTCAACATCGTCAAGAAGACCGAAGTCCAAACCGGTGGTTTCTCTAAGGAATCTATCTTGCCAAAGAGAAACTCTGACAAG | |
| | TTGATCGCCAGAAAGAAGGACTGGGACCCAAAGAAGTACGGTGGTTTCGACTCTCCAACCGTCGCCTACTCTGTCTTGGTCGTCGC | |
| | CAAGGTCGAAAAGGGTAAGTCTAAGAAGTTGAAGTCTGTCAAGGAATTGTTGGGTATCACCATCATGGAAAGATCTTCTTTCGAAA | |
| | AGAACCCAATCGACTTCTTGGAAGCCAAGGGTTACAAGGAAGTCAAGAAGGACTTGATCATCAAGTTGCCAAAGTACTCTTTGTTC | |
| | GAATTGGAAAACGGTAGAAAGAGAATGTTGGCCTCTGCCGGTGAATTGCAAAAGGGTAACGAATTGGCCTTGCCATCTAAGTACGT | |
| | CAACTTCTTGTACTTGGCCTCTCACTACGAAAAGTTGAAGGGTTCTCCAGAAGACAACGAACAAAAGCAATTGTTCGTCGAACAAC | |
| | ACAAGCACTACTTGGACGAAATCATCGAACAAATCTCTGAATTCTCTAAGAGAGTCATCTTGGCCGACGCCAACTTGGACAAGGTC | |
| | TTGTCTGCCTACAACAAGCACAGAGACAAGCCAATCAGAGAACAAGCCGAAAACATCATCCACTTGTTCACCTTGACCAACTTGGG | |
| | TGCCCCAGCCGCCTTCAAGTACTTCGACACCACCATCGACAGAAAGAGATACACCTCTACCAAGGAAGTCTTGGACGCCACCTTGA | |
| | TCCACCAATCTATCACCGGTTTGTACGAAACCAGAATCGACTTGTCTCAATTGGGTGGTGACGGTGGTGGTTCTCCAAAGAAGAAG | |
| | AGAAAGGTCTAA | |
| Cas9 ORF | ATGGATAAAAAATATTCTATTGGTTTAGATATTGGTACTAATTCTGTTGGTTGGGCTGTTATTACTGATGAATATAAAGTTCCTTC | 308 |
| using U rich | TAAAAAATTTAAAGTTTTAGGTAATACTGATCGTCATTCTATTAAAAAAAATTTAATTGGTGCTTTATTATTTGATTCTGGTGAAA | |
| codons of | CTGCTGAAGCTACTCGTTTAAAACGTACTGCTCGTCGTCGTTATACTCGTCGTAAAAATCGTATTTGTTATTTACAAGAAATTTTT | |
| Table 4, | TCTAATGAAATGGCTAAAGTTGATGATTCTTTTTTTCATCGTTTAGAAGAATCTTTTTTAGTTGAAGAAGATAAAAAACATGAACG | |
| with start | TCATCCTATTTTTGGTAATATTGTTGATGAAGTTGCTTATCATGAAAAATATCCTACTATTTATCATTTACGTAAAAAATTAGTTG | |
| and stop | ATTCTACTGATAAAGCTGATTTACGTTTAATTTATTTAGCTTTAGCTCATATGATTAAATTTCGTGGTCATTTTTTAATTGAAGGT | |
| codons | GATTTAAATCCTGATAATTCTGATGTTGATAAATTATTTATTCAATTAGTTCAAACTTATAATCAATTATTTGAAGAAAATCCTAT | |
| | TAATGCTTCTGGTGTTGATGCTAAAGCTATTTTATCTGCTCGTTTATCTAAATCTCGTCGTTTAGAAAATTTAATTGCTCAATTAC | |
| | CTGGTGAAAAAAAAAATGGTTTATTTGGTAATTTAATTGCTTTATCTTTAGGTTTAACTCCTAATTTTAAATCTAATTTTGATTTA | |
| | GCTGAAGATGCTAAATTACAATTATCTAAAGATACTTATGATGATGATTTAGATAATTTATTAGCTCAAATTGGTGATCAATATGC | |
| | TGATTTATTTTTAGCTGCTAAAAATTTATCTGATGCTATTTTATTATCTGATATTTTACGTGTTAATACTGAAATTACTAAAGCTC | |
| | CTTTATCTGCTTCTATGATTAAACGTTATGATGAACATCATCAAGATTTAACTTTATTAAAAGCTTTAGTTCGTCAACAATTACCT | |
| | GAAAAATATAAAGAAATTTTTTTTGATCAATCTAAAAATGGTTATGCTGGTTATATTGATGGTGGTGCTTCTCAAGAAGAATTTTA | |
| | TAAATTTATTAAACCTATTTTAGAAAAAATGGATGGTACTGAAGAATTATTAGTTAAATTAAATCGTGAAGATTTATTACGTAAAC | |
| | AACGTACTTTTGATAATGGTTCTATTCCTCATCAAATTCATTTAGGTGAATTACATGCTATTTTACGTCGTCAAGAAGATTTTTAT | |
| | CCTTTTTTAAAAGATAATCGTGAAAAAATTGAAAAAATTTTAACTTTTCGTATTCCTTATTATGTTGGTCCTTTAGCTCGTGGTAA | |
| | TTCTCGTTTTGCTTGGATGACTCGTAAATCTGAAGAAACTATTACTCCTTGGAATTTTGAAGAAGTTGTTGATAAAGGTGCTTCTG | |
| | CTCAATCTTTTATTGAACGTATGACTAATTTTGATAAAAATTTACCTAATGAAAAAGTTTTACCTAAACATTCTTTATTATATGAA | |
| | TATTTTACTGTTTATAATGAATTAACTAAAGTTAAATATGTTACTGAAGGTATGCGTAAACCTGCTTTTTTATCTGGTGAACAAAA | |
| | AAAAGCTATTGTTGATTTATTATTTAAAACTAATCGTAAAGTTACTGTTAAACAATTAAAAGAAGATTATTTTAAAAAAATTGAAT | |
| | GTTTTGATTCTGTTGAAATTTCTGGTGTTGAAGATCGTTTTAATGCTTCTTTAGGTACTTATCATGATTTATTAAAAATTATTAAA | |
| | GATAAAGATTTTTTAGATAATGAAGAAAATGAAGATATTTTAGAAGATATTGTTTTAACTTTAACTTTATTTGAAGATCGTGAAAT | |
| | GATTGAAGAACGTTTAAAAACTTATGCTCATTTATTTGATGATAAAGTTATGAAACAATTAAAACGTCGTCGTTATACTGGTTGGG | |
| | GTCGTTTATCTCGTAAATTAATTAATGGTATTCGTGATAAACAATCTGGTAAAACTATTTTAGATTTTTTAAAATCTGATGGTTTT | |
| | GCTAATCGTAATTTTATGCAATTAATTCATGATGATTCTTTAACTTTTAAAGAAGATATTCAAAAAGCTCAAGTTTCTGGTCAAGG | |
| | TGATTCTTTACATGAACATATTGCTAATTTAGCTGGTTCTCCTGCTATTAAAAAAGGTATTTTACAAACTGTTAAAGTTGTTGATG | |
| | AATTAGTTAAAGTTATGGGTCGTCATAAACCTGAAAATATTGTTATTGAAATGGCTCGTGAAAATCAAACTACTCAAAAAGGTCAA | |
| | AAAAATTCTCGTGAACGTATGAAACGTATTGAAGAAGGTATTAAAGAATTAGGTTCTCAAATTTTAAAAGAACATCCTGTTGAAAA | |
| | TACTCAATTACAAAATGAAAAATTATATTTATATTATTTACAAAATGGTCGTGATATGTATGTTGATCAAGAATTAGATATTAATC | |
| | GTTTATCTGATTATGATGTTGATCATATTGTTCCTCAATCTTTTTTAAAAGATGATTCTATTGATAATAAAGTTTTAACTCGTTCT | |
| | GATAAAAATCGTGGTAAATCTGATAATGTTCCTTCTGAAGAAGTTGTTAAAAAAATGAAAAATTATTGGCGTCAATTATTAAATGC | |
| | TAAATTAATTACTCAACGTAAATTTGATAATTTAACTAAAGCTGAACGTGGTGGTTTATCTGAATTAGATAAAGCTGGTTTTATTA | |
| | AACGTCAATTAGTTGAAACTCGTCAAATTACTAAACATGTTGCTCAAATTTTAGATTCTCGTATGAATACTAAATATGATGAAAAT | |
| | GATAAATTAATTCGTGAAGTTAAAGTTATTACTTTAAAATCTAAATTAGTTTCTGATTTTCGTAAAGATTTTCAATTTTATAAAGT | |
| | TCGTGAAATTAATAATTATCATCATGCTCATGATGCTTATTTAAATGCTGTTGTTGGTACTGCTTTAATTAAAAAATATCCTAAAT | |
| | TAGAATCTGAATTTGTTTATGGTGATTATAAAGTTTATGATGTTCGTAAAATGATTGCTAAATCTGAACAAGAAATTGGTAAAGCT | |
| | ACTGCTAAATATTTTTTTTATTCTAATATTATGAATTTTTTTAAAACTGAAATTACTTTAGCTAATGGTGAAATTCGTAAACGTCC | |
| | TTTAATTGAAACTAATGGTGAAACTGGTGAAATTGTTTGGGATAAAGGTCGTGATTTTGCTACTGTTCGTAAAGTTTTATCTATGC | |
| | CTCAAGTTAATATTGTTAAAAAAACTGAAGTTCAAACTGGTGGTTTTTCTAAAGAATCTATTTTACCTAAACGTAATTCTGATAAA | |
| | TTAATTGCTCGTAAAAAAGATTGGGATCCTAAAAAATATGGTGGTTTTGATTCTCCTACTGTTGCTTATTCTGTTTTAGTTGTTGC | |
| | TAAAGTTGAAAAAGGTAAATCTAAAAAATTAAAATCTGTTAAAGAATTATTAGGTATTACTATTATGGAACGTTCTTCTTTTGAAA | |
| | AAAATCCTATTGATTTTTTAGAAGCTAAAGGTTATAAAGAAGTTAAAAAAGATTTAATTATTAAATTACCTAAATATTCTTTATTT | |
| | GAATTAGAAAATGGTCGTAAACGTATGTTAGCTTCTGCTGGTGAATTACAAAAAGGTAATGAATTAGCTTTACCTTCTAAATATGT | |
| | TAATTTTTTATATTTAGCTTCTCATTATGAAAAATTAAAAGGTTCTCCTGAAGATAATGAACAAAAACAATTATTTGTTGAACAAC | |
| | ATAAACATTATTTAGATGAAATTATTGAACAAATTTCTGAATTTTCTAAACGTGTTATTTTAGCTGATGCTAATTTAGATAAAGTT | |
| | TTATCTGCTTATAATAAACATCGTGATAAACCTATTCGTGAACAAGCTGAAAATATTATTCATTTATTTACTTTAACTAATTTAGG | |
| | TGCTCCTGCTGCTTTTAAATATTTTGATACTACTATTGATCGTAAACGTTATACTTCTACTAAAGAAGTTTTAGATGCTACTTTAA | |
| | TTCATCAATCTATTACTGGTTTATATGAAACTCGTATTGATTTATCTCAATTAGGTGGTGATGGTGGTGGTTCTCCTAAAAAAAAA | |
| | CGTAAAGTTTGA | |
| Cas9 ORF | ATGGACAAAAAATACTCCATCGGCCTCGACATCGGCACCAACTCCGTCGGCTGGGCCGTCATCACCGACGAATACAAAGTCCCCTC | 309 |
| using low G | CAAAAAATTCAAAGTCCTCGGCAACACCGACAGACACTCCATCAAAAAAAACCTCATCGGCGCCCTCCTCTTCGACTCCGGCGAAA | |
| codons of | CCGCCGAAGCCACCAGACTCAAAAGAACCGCCAGAAGAAGATACACCAGAAGAAAAAACAGAATCTGCTACCTCCAAGAAATCTTC | |
| Table 4, | TCCAACGAAATGGCCAAAGTCGACGACTCCTTCTTCCACAGACTCGAAGAATCCTTCCTCGTCGAAGAAGACAAAAAACACGAAAG | |
| with start | ACACCCCATCTTCGGCAACATCGTCGACGAAGTCGCCTACCACGAAAAATACCCCACCATCTACCACCTCAGAAAAAAACTCGTCG | |
| and stop | ACTCCACCGACAAAGCCGACCTCAGACTCATCTACCTCGCCCTCGCCCACATGATCAAATTCAGAGGCCACTTCCTCATCGAAGGC | |
| codons | GACCTCAACCCCGACAACTCCGACGTCGACAAACTCTTCATCCAACTCGTCCAAACCTACAACCAACTCTTCGAAGAAAACCCCAT | |
| | CAACGCCTCCGGCGTCGACGCCAAAGCCATCCTCTCCGCCAGACTCTCCAAATCCAGAAGACTCGAAAACCTCATCGCCCAACTCC | |
| | CCGGCGAAAAAAAAAACGGCCTCTTCGGCAACCTCATCGCCCTCTCCCTCGGCCTCACCCCCAACTTCAAATCCAACTTCGACCTC | |
| | GCCGAAGACGCCAAACTCCAACTCTCCAAAGACACCTACGACGACGACCTCGACAACCTCCTCGCCCAAATCGGCGACCAATACGC | |
| | CGACCTCTTCCTCGCCGCCAAAAACCTCTCCGACGCCATCCTCCTCTCCGACATCCTCAGAGTCAACACCGAAATCACCAAAGCCC | |
| | CCCTCTCCGCCTCCATGATCAAAAGATACGACGAACACCACCAAGACCTCACCCTCCTCAAAGCCCTCGTCAGACAACAACTCCCC | |
| | GAAAAATACAAAGAAATCTTCTTCGACCAATCCAAAAACGGCTACGCCGGCTACATCGACGGCGGCGCCTCCCAAGAAGAATTCTA | |
| | CAAATTCATCAAACCCATCCTCGAAAAAATGGACGGCACCGAAGAACTCCTCGTCAAACTCAACAGAGAAGACCTCCTCAGAAAAC | |
| | AAAGAACCTTCGACAACGGCTCCATCCCCCACCAAATCCACCTCGGCGAACTCCACGCCATCCTCAGAAGACAAGAAGACTTCTAC | |
| | CCCTTCCTCAAAGACAACAGAGAAAAAATCGAAAAAATCCTCACCTTCAGAATCCCCTACTACGTCGGCCCCCTCGCCAGAGGCAA | |
| | CTCCAGATTCGCCTGGATGACCAGAAAATCCGAAGAAACCATCACCCCCTGGAACTTCGAAGAAGTCGTCGACAAAGGCGCCTCCG | |
| | CCCAATCCTTCATCGAAAGAATGACCAACTTCGACAAAAACCTCCCCAACGAAAAAGTCCTCCCCAAACACTCCCTCCTCTACGAA | |
| | TACTTCACCGTCTACAACGAACTCACCAAAGTCAAATACGTCACCGAAGGCATGAGAAAACCCGCCTTCCTCTCCGGCGAACAAAA | |
| | AAAAGCCATCGTCGACCTCCTCTTCAAAACCAACAGAAAAGTCACCGTCAAACAACTCAAAGAAGACTACTTCAAAAAAATCGAAT | |
| | GCTTCGACTCCGTCGAAATCTCCGGCGTCGAAGACAGATTCAACGCCTCCCTCGGCACCTACCACGACCTCCTCAAAATCATCAAA | |
| | GACAAAGACTTCCTCGACAACGAAGAAAACGAAGACATCCTCGAAGACATCGTCCTCACCCTCACCCTCTTCGAAGACAGAGAAAT | |
| | GATCGAAGAAAGACTCAAAACCTACGCCCACCTCTTCGACGACAAAGTCATGAAACAACTCAAAAGAAGAAGATACACCGGCTGGG | |
| | GCAGACTCTCCAGAAAACTCATCAACGGCATCAGAGACAAACAATCCGGCAAAACCATCCTCGACTTCCTCAAATCCGACGGCTTC | |
| | GCCAACAGAAACTTCATGCAACTCATCCACGACGACTCCCTCACCTTCAAAGAAGACATCCAAAAAGCCCAAGTCTCCGGCCAAGG | |
| | CGACTCCCTCCACGAACACATCGCCAACCTCGCCGGCTCCCCCGCCATCAAAAAAGGCATCCTCCAAACCGTCAAAGTCGTCGACG | |
| | AACTCGTCAAAGTCATGGGCAGACACAAACCCGAAAACATCGTCATCGAAATGGCCAGAGAAAACCAAACCACCCAAAAAGGCCAA | |
| | AAAAACTCCAGAGAAAGAATGAAAAGAATCGAAGAAGGCATCAAAGAACTCGGCTCCCAAATCCTCAAAGAACACCCCGTCGAAAA | |
| | CACCCAACTCCAAAACGAAAAACTCTACCTCTACTACCTCCAAAACGGCAGAGACATGTACGTCGACCAAGAACTCGACATCAACA | |
| | GACTCTCCGACTACGACGTCGACCACATCGTCCCCCAATCCTTCCTCAAAGACGACTCCATCGACAACAAAGTCCTCACCAGATCC | |
| | GACAAAAACAGAGGCAAATCCGACAACGTCCCCTCCGAAGAAGTCGTCAAAAAAATGAAAAACTACTGGAGACAACTCCTCAACGC | |
| | CAAACTCATCACCCAAAGAAAATTCGACAACCTCACCAAAGCCGAAAGAGGCGGCCTCTCCGAACTCGACAAAGCCGGCTTCATCA | |
| | AAAGACAACTCGTCGAAACCAGACAAATCACCAAACACGTCGCCCAAATCCTCGACTCCAGAATGAACACCAAATACGACGAAAAC | |
| | GACAAACTCATCAGAGAAGTCAAAGTCATCACCCTCAAATCCAAACTCGTCTCCGACTTCAGAAAAGACTTCCAATTCTACAAAGT | |
| | CAGAGAAATCAACAACTACCACCACGCCCACGACGCCTACCTCAACGCCGTCGTCGGCACCGCCCTCATCAAAAAATACCCCAAAC | |
| | TCGAATCCGAATTCGTCTACGGCGACTACAAAGTCTACGACGTCAGAAAAATGATCGCCAAATCCGAACAAGAAATCGGCAAAGCC | |
| | ACCGCCAAATACTTCTTCTACTCCAACATCATGAACTTCTTCAAAACCGAAATCACCCTCGCCAACGGCGAAATCAGAAAAAGACC | |
| | CCTCATCGAAACCAACGGCGAAACCGGCGAAATCGTCTGGGACAAAGGCAGAGACTTCGCCACCGTCAGAAAAGTCCTCTCCATGC | |
| | CCCAAGTCAACATCGTCAAAAAAACCGAAGTCCAAACCGGCGGCTTCTCCAAAGAATCCATCCTCCCCAAAAGAAACTCCGACAAA | |
| | CTCATCGCCAGAAAAAAAGACTGGGACCCCAAAAAATACGGCGGCTTCGACTCCCCCACCGTCGCCTACTCCGTCCTCGTCGTCGC | |
| | CAAAGTCGAAAAAGGCAAATCCAAAAAACTCAAATCCGTCAAAGAACTCCTCGGCATCACCATCATGGAAAGATCCTCCTTCGAAA | |
| | AAAACCCCATCGACTTCCTCGAAGCCAAAGGCTACAAAGAAGTCAAAAAAGACCTCATCATCAAACTCCCCAAATACTCCCTCTTC | |
| | GAACTCGAAAACGGCAGAAAAAGAATGCTCGCCTCCGCCGGCGAACTCCAAAAAGGCAACGAACTCGCCCTCCCCTCCAAATACGT | |
| | CAACTTCCTCTACCTCGCCTCCCACTACGAAAAACTCAAAGGCTCCCCCGAAGACAACGAACAAAAACAACTCTTCGTCGAACAAC | |
| | ACAAACACTACCTCGACGAAATCATCGAACAAATCTCCGAATTCTCCAAAAGAGTCATCCTCGCCGACGCCAACCTCGACAAAGTC | |
| | CTCTCCGCCTACAACAAACACAGAGACAAACCCATCAGAGAACAAGCCGAAAACATCATCCACCTCTTCACCCTCACCAACCTCGG | |
| | CGCCCCCGCCGCCTTCAAATACTTCGACACCACCATCGACAGAAAAAGATACACCTCCACCAAAGAAGTCCTCGACGCCACCCTCA | |
| | TCCACCAATCCATCACCGGCCTCTACGAAACCAGAATCGACCTCTCCCAACTCGGCGGCGACGGCGGCGGCTCCCCCAAAAAAAAA | |
| | AGAAAAGTCTGA | |
| Cas9 ORF | ATGGATAAGAAGTATAGTATTGGATTGGATATTGGAACAAATAGTGTGGGATGGGCTGTGATTACAGATGAGTATAAGGTGCCTAG | 310 |
| using low C | TAAGAAGTTTAAGGTGTTGGGAAATACAGATAGACATAGTATTAAGAAGAATTTGATTGGAGCTTTGTTGTTTGATAGTGGAGAGA | |
| codons of | CAGCTGAGGCTACAAGATTGAAGAGAACAGCTAGAAGAAGATATACAAGAAGAAAGAATAGAATTTGTTATTTGCAGGAGATTTTT | |
| Table 4, | AGTAATGAGATGGCTAAGGTGGATGATAGTTTTTTTCATAGATTGGAGGAGAGTTTTTTGGTGGAGGAGGATAAGAAGCATGAGAG | |
| with start | ACATCCTATTTTTGGAAATATTGTGGATGAGGTGGCTTATCATGAGAAGTATCCTACAATTTATCATTTGAGAAAGAAGTTGGTGG | |
| and stop | ATAGTACAGATAAGGCTGATTTGAGATTGATTTATTTGGCTTTGGCTCATATGATTAAGTTTAGAGGACATTTTTTGATTGAGGGA | |
| codons | GATTTGAATCCTGATAATAGTGATGTGGATAAGTTGTTTATTCAGTTGGTGCAGACATATAATCAGTTGTTTGAGGAGAATCCTAT | |
| | TAATGCTAGTGGAGTGGATGCTAAGGCTATTTTGAGTGCTAGATTGAGTAAGAGTAGAAGATTGGAGAATTTGATTGCTCAGTTGC | |
| | CTGGAGAGAAGAAGAATGGATTGTTTGGAAATTTGATTGCTTTGAGTTTGGGATTGACACCTAATTTTAAGAGTAATTTTGATTTG | |
| | GCTGAGGATGCTAAGTTGCAGTTGAGTAAGGATACATATGATGATGATTTGGATAATTTGTTGGCTCAGATTGGAGATCAGTATGC | |
| | TGATTTGTTTTTGGCTGCTAAGAATTTGAGTGATGCTATTTTGTTGAGTGATATTTTGAGAGTGAATACAGAGATTACAAAGGCTC | |
| | CTTTGAGTGCTAGTATGATTAAGAGATATGATGAGCATCATCAGGATTTGACATTGTTGAAGGCTTTGGTGAGACAGCAGTTGCCT | |
| | GAGAAGTATAAGGAGATTTTTTTTGATCAGAGTAAGAATGGATATGCTGGATATATTGATGGAGGAGCTAGTCAGGAGGAGTTTTA | |
| | TAAGTTTATTAAGCCTATTTTGGAGAAGATGGATGGAACAGAGGAGTTGTTGGTGAAGTTGAATAGAGAGGATTTGTTGAGAAAGC | |
| | AGAGAACATTTGATAATGGAAGTATTCCTCATCAGATTCATTTGGGAGAGTTGCATGCTATTTTGAGAAGACAGGAGGATTTTTAT | |
| | CCTTTTTTGAAGGATAATAGAGAGAAGATTGAGAAGATTTTGACATTTAGAATTCCTTATTATGTGGGACCTTTGGCTAGAGGAAA | |
| | TAGTAGATTTGCTTGGATGACAAGAAAGAGTGAGGAGACAATTACACCTTGGAATTTTGAGGAGGTGGTGGATAAGGGAGCTAGTG | |
| | CTCAGAGTTTTATTGAGAGAATGACAAATTTTGATAAGAATTTGCCTAATGAGAAGGTGTTGCCTAAGCATAGTTTGTTGTATGAG | |
| | TATTTTACAGTGTATAATGAGTTGACAAAGGTGAAGTATGTGACAGAGGGAATGAGAAAGCCTGCTTTTTTGAGTGGAGAGCAGAA | |
| | GAAGGCTATTGTGGATTTGTTGTTTAAGACAAATAGAAAGGTGACAGTGAAGCAGTTGAAGGAGGATTATTTTAAGAAGATTGAGT | |
| | GTTTTGATAGTGTGGAGATTAGTGGAGTGGAGGATAGATTTAATGCTAGTTTGGGAACATATCATGATTTGTTGAAGATTATTAAG | |
| | GATAAGGATTTTTTGGATAATGAGGAGAATGAGGATATTTTGGAGGATATTGTGTTGACATTGACATTGTTTGAGGATAGAGAGAT | |
| | GATTGAGGAGAGATTGAAGACATATGCTCATTTGTTTGATGATAAGGTGATGAAGCAGTTGAAGAGAAGAAGATATACAGGATGGG | |
| | GAAGATTGAGTAGAAAGTTGATTAATGGAATTAGAGATAAGCAGAGTGGAAAGACAATTTTGGATTTTTTGAAGAGTGATGGATTT | |
| | GCTAATAGAAATTTTATGCAGTTGATTCATGATGATAGTTTGACATTTAAGGAGGATATTCAGAAGGCTCAGGTGAGTGGACAGGG | |
| | AGATAGTTTGCATGAGCATATTGCTAATTTGGCTGGAAGTCCTGCTATTAAGAAGGGAATTTTGCAGACAGTGAAGGTGGTGGATG | |
| | AGTTGGTGAAGGTGATGGGAAGACATAAGCCTGAGAATATTGTGATTGAGATGGCTAGAGAGAATCAGACAACACAGAAGGGACAG | |
| | AAGAATAGTAGAGAGAGAATGAAGAGAATTGAGGAGGGAATTAAGGAGTTGGGAAGTCAGATTTTGAAGGAGCATCCTGTGGAGAA | |
| | TACACAGTTGCAGAATGAGAAGTTGTATTTGTATTATTTGCAGAATGGAAGAGATATGTATGTGGATCAGGAGTTGGATATTAATA | |
| | GATTGAGTGATTATGATGTGGATCATATTGTGCCTCAGAGTTTTTTGAAGGATGATAGTATTGATAATAAGGTGTTGACAAGAAGT | |
| | GATAAGAATAGAGGAAAGAGTGATAATGTGCCTAGTGAGGAGGTGGTGAAGAAGATGAAGAATTATTGGAGACAGTTGTTGAATGC | |
| | TAAGTTGATTACACAGAGAAAGTTTGATAATTTGACAAAGGCTGAGAGAGGAGGATTGAGTGAGTTGGATAAGGCTGGATTTATTA | |
| | AGAGACAGTTGGTGGAGACAAGACAGATTACAAAGCATGTGGCTCAGATTTTGGATAGTAGAATGAATACAAAGTATGATGAGAAT | |
| | GATAAGTTGATTAGAGAGGTGAAGGTGATTACATTGAAGAGTAAGTTGGTGAGTGATTTTAGAAAGGATTTTCAGTTTTATAAGGT | |
| | GAGAGAGATTAATAATTATCATCATGCTCATGATGCTTATTTGAATGCTGTGGTGGGAACAGCTTTGATTAAGAAGTATCCTAAGT | |
| | TGGAGAGTGAGTTTGTGTATGGAGATTATAAGGTGTATGATGTGAGAAAGATGATTGCTAAGAGTGAGCAGGAGATTGGAAAGGCT | |
| | ACAGCTAAGTATTTTTTTTATAGTAATATTATGAATTTTTTTAAGACAGAGATTACATTGGCTAATGGAGAGATTAGAAAGAGACC | |
| | TTTGATTGAGACAAATGGAGAGACAGGAGAGATTGTGTGGGATAAGGGAAGAGATTTTGCTACAGTGAGAAAGGTGTTGAGTATGC | |
| | CTCAGGTGAATATTGTGAAGAAGACAGAGGTGCAGACAGGAGGATTTAGTAAGGAGAGTATTTTGCCTAAGAGAAATAGTGATAAG | |
| | TTGATTGCTAGAAAGAAGGATTGGGATCCTAAGAAGTATGGAGGATTTGATAGTCCTACAGTGGCTTATAGTGTGTTGGTGGTGGC | |
| | TAAGGTGGAGAAGGGAAAGAGTAAGAAGTTGAAGAGTGTGAAGGAGTTGTTGGGAATTACAATTATGGAGAGAAGTAGTTTTGAGA | |
| | AGAATCCTATTGATTTTTTGGAGGCTAAGGGATATAAGGAGGTGAAGAAGGATTTGATTATTAAGTTGCCTAAGTATAGTTTGTTT | |
| | GAGTTGGAGAATGGAAGAAAGAGAATGTTGGCTAGTGCTGGAGAGTTGCAGAAGGGAAATGAGTTGGCTTTGCCTAGTAAGTATGT | |
| | GAATTTTTTGTATTTGGCTAGTCATTATGAGAAGTTGAAGGGAAGTCCTGAGGATAATGAGCAGAAGCAGTTGTTTGTGGAGCAGC | |
| | ATAAGCATTATTTGGATGAGATTATTGAGCAGATTAGTGAGTTTAGTAAGAGAGTGATTTTGGCTGATGCTAATTTGGATAAGGTG | |
| | TTGAGTGCTTATAATAAGCATAGAGATAAGCCTATTAGAGAGCAGGCTGAGAATATTATTCATTTGTTTACATTGACAAATTTGGG | |
| | AGCTCCTGCTGCTTTTAAGTATTTTGATACAACAATTGATAGAAAGAGATATACAAGTACAAAGGAGGTGTTGGATGCTACATTGA | |
| | TTCATCAGAGTATTACAGGATTGTATGAGACAAGAATTGATTTGAGTCAGTTGGGAGGAGATGGAGGAGGAAGTCCTAAGAAGAAG | |
| | AGAAAGGTGTGA | |
| Cas9 ORF | ATGGACAAGAAGTACTCCATCGGCCTGGACATCGGCACCAACTCCGTGGGCTGGGCCGTGATCACCGACGAGTACAAGGTGCCCTC | 311 |
| using low A | CAAGAAGTTCAAGGTGCTGGGCAACACCGACCGGCACTCCATCAAGAAGAACCTGATCGGCGCCCTGCTGTTCGACTCCGGCGAGA | |
| codons of | CCGCCGAGGCCACCCGGCTGAAGCGGACCGCCCGGCGGCGGTACACCCGGCGGAAGAACCGGATCTGCTACCTGCAGGAGATCTTC | |
| Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACTCCACCGACAAGGCCGACCTGCGGCTGATCTACCTGGCCCTGGCCCACATGATCAAGTTCCGGGGCCACTTCCTGATCGAGGGC | |
| codons | GACCTGAACCCCGACAACTCCGACGTGGACAAGCTGTTCATCCAGCTGGTGCAGACCTACAACCAGCTGTTCGAGGAGAACCCCAT | |
| | CAACGCCTCCGGCGTGGACGCCAAGGCCATCCTGTCCGCCCGGCTGTCCAAGTCCCGGCGGCTGGAGAACCTGATCGCCCAGCTGC | |
| | CCGGCGAGAAGAAGAACGGCCTGTTCGGCAACCTGATCGCCCTGTCCCTGGGCCTGACCCCCAACTTCAAGTCCAACTTCGACCTG | |
| | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACCACATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCGGCGGCTCCCCCAAGAAGAAG | |
| | CGGAAGGTGTGA | |
| Cas9 ORF | ATGGACAAGAAGTACAGCATCGGCCTGGACATCGGCACCAACAGCGTGGGCTGGGCCGTGATCACCGACGAGTACAAGGTGCCCAG | 312 |
| using low | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACAGCACCGACAAGGCCGACCTGCGGCTGATCTACCTGGCCCTGGCCCACATGATCAAGTTCCGGGGCCACTTCCTGATCGAGGGC | |
| codons | GACCTGAACCCCGACAACAGCGACGTGGACAAGCTGTTCATCCAGCTGGTGCAGACCTACAACCAGCTGTTCGAGGAGAACCCCAT | |
| | CAACGCCAGCGGCGTGGACGCCAAGGCCATCCTGAGCGCCCGGCTGAGCAAGAGCCGGCGGCTGGAGAACCTGATCGCCCAGCTGC | |
| | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCGGCGGCAGCCCCAAGAAGAAG | |
| | CGGAAGGTGTGA | |
| Cas9 ORF | ATGGACAAGAAGTACTCCATCGGCCTGGACATCGGCACCAACTCCGTGGGCTGGGCCGTGATCACCGACGAGTACAAGGTGCCCTC | 313 |
| using low A | CAAGAAGTTCAAGGTGCTGGGCAACACCGACCGGCACTCCATCAAGAAGAACCTGATCGGCGCCCTGCTGTTCGACTCCGGCGAGA | |
| codons of | CCGCCGAGGCCACCCGGCTGAAGCGGACCGCCCGGCGGCGGTACACCCGGCGGAAGAACCGGATCTGCTACCTGCAGGAGATCTTC | |
| Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with two C- | GCACCCCATCTTCGGCAACATCGTGGACGAGGTGGCCTACCACGAGAAGTACCCCACCATCTACCACCTGCGGAAGAAGCTGGTGG | |
| terminal NLS | ACTCCACCGACAAGGCCGACCTGCGGCTGATCTACCTGGCCCTGGCCCACATGATCAAGTTCCGGGGCCACTTCCTGATCGAGGGC | |
| sequences | GACCTGAACCCCGACAACTCCGACGTGGACAAGCTGTTCATCCAGCTGGTGCAGACCTACAACCAGCTGTTCGAGGAGAACCCCAT | |
| and start | CAACGCCTCCGGCGTGGACGCCAAGGCCATCCTGTCCGCCCGGCTGTCCAAGTCCCGGCGGCTGGAGAACCTGATCGCCCAGCTGC | |
| and stop | CCGGCGAGAAGAAGAACGGCCTGTTCGGCAACCTGATCGCCCTGTCCCTGGGCCTGACCCCCAACTTCAAGTCCAACTTCGACCTG | |
| codons | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACCACATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCTCCGGCTCCCCCAAGAAGAAG | |
| | CGGAAGGTGGACGGCTCCCCCAAGAAGAAGCGGAAGGTGGACTCCGGCTGA | |
| Cas9 nickase | ATGGACAAGAAGTACTCCATCGGCCTGGCCATCGGCACCAACTCCGTGGGCTGGGCCGTGATCACCGACGAGTACAAGGTGCCCTC | 314 |
| ORF using | CAAGAAGTTCAAGGTGCTGGGCAACACCGACCGGCACTCCATCAAGAAGAACCTGATCGGCGCCCTGCTGTTCGACTCCGGCGAGA | |
| low A codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACTCCACCGACAAGGCCGACCTGCGGCTGATCTACCTGGCCCTGGCCCACATGATCAAGTTCCGGGGCCACTTCCTGATCGAGGGC | |
| codons | GACCTGAACCCCGACAACTCCGACGTGGACAAGCTGTTCATCCAGCTGGTGCAGACCTACAACCAGCTGTTCGAGGAGAACCCCAT | |
| | CAACGCCTCCGGCGTGGACGCCAAGGCCATCCTGTCCGCCCGGCTGTCCAAGTCCCGGCGGCTGGAGAACCTGATCGCCCAGCTGC | |
| | CCGGCGAGAAGAAGAACGGCCTGTTCGGCAACCTGATCGCCCTGTCCCTGGGCCTGACCCCCAACTTCAAGTCCAACTTCGACCTG | |
| | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACCACATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCGGCGGCTCCCCCAAGAAGAAG | |
| | CGGAAGGTGTGA | |
| Cas9 nickase | ATGGACAAGAAGTACTCCATCGGCCTGGCCATCGGCACCAACTCCGTGGGCTGGGCCGTGATCACCGACGAGTACAAGGTGCCCTC | 315 |
| ORF using | CAAGAAGTTCAAGGTGCTGGGCAACACCGACCGGCACTCCATCAAGAAGAACCTGATCGGCGCCCTGCTGTTCGACTCCGGCGAGA | |
| low A codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACTCCACCGACAAGGCCGACCTGCGGCTGATCTACCTGGCCCTGGCCCACATGATCAAGTTCCGGGGCCACTTCCTGATCGAGGGC | |
| codons and | GACCTGAACCCCGACAACTCCGACGTGGACAAGCTGTTCATCCAGCTGGTGCAGACCTACAACCAGCTGTTCGAGGAGAACCCCAT | |
| no NLS | CAACGCCTCCGGCGTGGACGCCAAGGCCATCCTGTCCGCCCGGCTGTCCAAGTCCCGGCGGCTGGAGAACCTGATCGCCCAGCTGC | |
| | CCGGCGAGAAGAAGAACGGCCTGTTCGGCAACCTGATCGCCCTGTCCCTGGGCCTGACCCCCAACTTCAAGTCCAACTTCGACCTG | |
| | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACCACATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACTGA | |
| Cas9 nickase | ATGGACAAGAAGTACTCCATCGGCCTGGCCATCGGCACCAACTCCGTGGGCTGGGCCGTGATCACCGACGAGTACAAGGTGCCCTC | 316 |
| ORF using | CAAGAAGTTCAAGGTGCTGGGCAACACCGACCGGCACTCCATCAAGAAGAACCTGATCGGCGCCCTGCTGTTCGACTCCGGCGAGA | |
| low A codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with two C- | GCACCCCATCTTCGGCAACATCGTGGACGAGGTGGCCTACCACGAGAAGTACCCCACCATCTACCACCTGCGGAAGAAGCTGGTGG | |
| terminal NLS | ACTCCACCGACAAGGCCGACCTGCGGCTGATCTACCTGGCCCTGGCCCACATGATCAAGTTCCGGGGCCACTTCCTGATCGAGGGC | |
| sequences | GACCTGAACCCCGACAACTCCGACGTGGACAAGCTGTTCATCCAGCTGGTGCAGACCTACAACCAGCTGTTCGAGGAGAACCCCAT | |
| and start | CAACGCCTCCGGCGTGGACGCCAAGGCCATCCTGTCCGCCCGGCTGTCCAAGTCCCGGCGGCTGGAGAACCTGATCGCCCAGCTGC | |
| and stop | CCGGCGAGAAGAAGAACGGCCTGTTCGGCAACCTGATCGCCCTGTCCCTGGGCCTGACCCCCAACTTCAAGTCCAACTTCGACCTG | |
| codons | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACCACATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCTCCGGCTCCCCCAAGAAGAAG | |
| | CGGAAGGTGGACGGCTCCCCCAAGAAGAAGCGGAAGGTGGACTCCGGCTGA | |
| dCas9 ORF | ATGGACAAGAAGTACTCCATCGGCCTGGCCATCGGCACCAACTCCGTGGGCTGGGCCGTGATCACCGACGAGTACAAGGTGCCCTC | 317 |
| using low A | CAAGAAGTTCAAGGTGCTGGGCAACACCGACCGGCACTCCATCAAGAAGAACCTGATCGGCGCCCTGCTGTTCGACTCCGGCGAGA | |
| codons of | CCGCCGAGGCCACCCGGCTGAAGCGGACCGCCCGGCGGCGGTACACCCGGCGGAAGAACCGGATCTGCTACCTGCAGGAGATCTTC | |
| Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACTCCACCGACAAGGCCGACCTGCGGCTGATCTACCTGGCCCTGGCCCACATGATCAAGTTCCGGGGCCACTTCCTGATCGAGGGC | |
| codons | GACCTGAACCCCGACAACTCCGACGTGGACAAGCTGTTCATCCAGCTGGTGCAGACCTACAACCAGCTGTTCGAGGAGAACCCCAT | |
| | CAACGCCTCCGGCGTGGACGCCAAGGCCATCCTGTCCGCCCGGCTGTCCAAGTCCCGGCGGCTGGAGAACCTGATCGCCCAGCTGC | |
| | CCGGCGAGAAGAAGAACGGCCTGTTCGGCAACCTGATCGCCCTGTCCCTGGGCCTGACCCCCAACTTCAAGTCCAACTTCGACCTG | |
| | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACGCCATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCGGCGGCTCCCCCAAGAAGAAG | |
| | CGGAAGGTGTGA | |
| dCas9 ORF | ATGGACAAGAAGTACTCCATCGGCCTGGCCATCGGCACCAACTCCGTGGGCTGGGCCGTGATCACCGACGAGTACAAGGTGCCCTC | 318 |
| using low A | CAAGAAGTTCAAGGTGCTGGGCAACACCGACCGGCACTCCATCAAGAAGAACCTGATCGGCGCCCTGCTGTTCGACTCCGGCGAGA | |
| codons of | CCGCCGAGGCCACCCGGCTGAAGCGGACCGCCCGGCGGCGGTACACCCGGCGGAAGAACCGGATCTGCTACCTGCAGGAGATCTTC | |
| Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACTCCACCGACAAGGCCGACCTGCGGCTGATCTACCTGGCCCTGGCCCACATGATCAAGTTCCGGGGCCACTTCCTGATCGAGGGC | |
| codons and | GACCTGAACCCCGACAACTCCGACGTGGACAAGCTGTTCATCCAGCTGGTGCAGACCTACAACCAGCTGTTCGAGGAGAACCCCAT | |
| no NLS | CAACGCCTCCGGCGTGGACGCCAAGGCCATCCTGTCCGCCCGGCTGTCCAAGTCCCGGCGGCTGGAGAACCTGATCGCCCAGCTGC | |
| | CCGGCGAGAAGAAGAACGGCCTGTTCGGCAACCTGATCGCCCTGTCCCTGGGCCTGACCCCCAACTTCAAGTCCAACTTCGACCTG | |
| | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACGCCATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACTGA | |
| dCas9 ORF | ATGGACAAGAAGTACTCCATCGGCCTGGCCATCGGCACCAACTCCGTGGGCTGGGCCGTGATCACCGACGAGTACAAGGTGCCCTC | 319 |
| using low A | CAAGAAGTTCAAGGTGCTGGGCAACACCGACCGGCACTCCATCAAGAAGAACCTGATCGGCGCCCTGCTGTTCGACTCCGGCGAGA | |
| codons of | CCGCCGAGGCCACCCGGCTGAAGCGGACCGCCCGGCGGCGGTACACCCGGCGGAAGAACCGGATCTGCTACCTGCAGGAGATCTTC | |
| Table 4, | TCCAACGAGATGGCCAAGGTGGACGACTCCTTCTTCCACCGGCTGGAGGAGTCCTTCCTGGTGGAGGAGGACAAGAAGCACGAGCG | |
| with two C- | GCACCCCATCTTCGGCAACATCGTGGACGAGGTGGCCTACCACGAGAAGTACCCCACCATCTACCACCTGCGGAAGAAGCTGGTGG | |
| terminal NLS | ACTCCACCGACAAGGCCGACCTGCGGCTGATCTACCTGGCCCTGGCCCACATGATCAAGTTCCGGGGCCACTTCCTGATCGAGGGC | |
| sequences | GACCTGAACCCCGACAACTCCGACGTGGACAAGCTGTTCATCCAGCTGGTGCAGACCTACAACCAGCTGTTCGAGGAGAACCCCAT | |
| and start | CAACGCCTCCGGCGTGGACGCCAAGGCCATCCTGTCCGCCCGGCTGTCCAAGTCCCGGCGGCTGGAGAACCTGATCGCCCAGCTGC | |
| and stop | CCGGCGAGAAGAAGAACGGCCTGTTCGGCAACCTGATCGCCCTGTCCCTGGGCCTGACCCCCAACTTCAAGTCCAACTTCGACCTG | |
| codons | GCCGAGGACGCCAAGCTGCAGCTGTCCAAGGACACCTACGACGACGACCTGGACAACCTGCTGGCCCAGATCGGCGACCAGTACGC | |
| | CGACCTGTTCCTGGCCGCCAAGAACCTGTCCGACGCCATCCTGCTGTCCGACATCCTGCGGGTGAACACCGAGATCACCAAGGCCC | |
| | CCCTGTCCGCCTCCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGTCCAAGAACGGCTACGCCGGCTACATCGACGGCGGCGCCTCCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCTCCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CTCCCGGTTCGCCTGGATGACCCGGAAGTCCGAGGAGACCATCACCCCCTGGAACTTCGAGGAGGTGGTGGACAAGGGCGCCTCCG | |
| | CCCAGTCCTTCATCGAGCGGATGACCAACTTCGACAAGAACCTGCCCAACGAGAAGGTGCTGCCCAAGCACTCCCTGCTGTACGAG | |
| | TACTTCACCGTGTACAACGAGCTGACCAAGGTGAAGTACGTGACCGAGGGCATGCGGAAGCCCGCCTTCCTGTCCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACTCCGTGGAGATCTCCGGCGTGGAGGACCGGTTCAACGCCTCCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGTCCCGGAAGCTGATCAACGGCATCCGGGACAAGCAGTCCGGCAAGACCATCCTGGACTTCCTGAAGTCCGACGGCTTC | |
| | GCCAACCGGAACTTCATGCAGCTGATCCACGACGACTCCCTGACCTTCAAGGAGGACATCCAGAAGGCCCAGGTGTCCGGCCAGGG | |
| | CGACTCCCTGCACGAGCACATCGCCAACCTGGCCGGCTCCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACTCCCGGGAGCGGATGAAGCGGATCGAGGAGGGCATCAAGGAGCTGGGCTCCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGTCCGACTACGACGTGGACGCCATCGTGCCCCAGTCCTTCCTGAAGGACGACTCCATCGACAACAAGGTGCTGACCCGGTCC | |
| | GACAAGAACCGGGGCAAGTCCGACAACGTGCCCTCCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGTCCGAGCTGGACAAGGCCGGCTTCATCA | |
| | AGCGGCAGCTGGTGGAGACCCGGCAGATCACCAAGCACGTGGCCCAGATCCTGGACTCCCGGATGAACACCAAGTACGACGAGAAC | |
| | GACAAGCTGATCCGGGAGGTGAAGGTGATCACCCTGAAGTCCAAGCTGGTGTCCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGTCCGAGTTCGTGTACGGCGACTACAAGGTGTACGACGTGCGGAAGATGATCGCCAAGTCCGAGCAGGAGATCGGCAAGGCC | |
| | ACCGCCAAGTACTTCTTCTACTCCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGTCCATGC | |
| | CCCAGGTGAACATCGTGAAGAAGACCGAGGTGCAGACCGGCGGCTTCTCCAAGGAGTCCATCCTGCCCAAGCGGAACTCCGACAAG | |
| | CTGATCGCCCGGAAGAAGGACTGGGACCCCAAGAAGTACGGCGGCTTCGACTCCCCCACCGTGGCCTACTCCGTGCTGGTGGTGGC | |
| | CAAGGTGGAGAAGGGCAAGTCCAAGAAGCTGAAGTCCGTGAAGGAGCTGCTGGGCATCACCATCATGGAGCGGTCCTCCTTCGAGA | |
| | AGAACCCCATCGACTTCCTGGAGGCCAAGGGCTACAAGGAGGTGAAGAAGGACCTGATCATCAAGCTGCCCAAGTACTCCCTGTTC | |
| | GAGCTGGAGAACGGCCGGAAGCGGATGCTGGCCTCCGCCGGCGAGCTGCAGAAGGGCAACGAGCTGGCCCTGCCCTCCAAGTACGT | |
| | GAACTTCCTGTACCTGGCCTCCCACTACGAGAAGCTGAAGGGCTCCCCCGAGGACAACGAGCAGAAGCAGCTGTTCGTGGAGCAGC | |
| | ACAAGCACTACCTGGACGAGATCATCGAGCAGATCTCCGAGTTCTCCAAGCGGGTGATCCTGGCCGACGCCAACCTGGACAAGGTG | |
| | CTGTCCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCTCCACCAAGGAGGTGCTGGACGCCACCCTGA | |
| | TCCACCAGTCCATCACCGGCCTGTACGAGACCCGGATCGACCTGTCCCAGCTGGGCGGCGACGGCTCCGGCTCCCCCAAGAAGAAG | |
| | CGGAAGGTGGACGGCTCCCCCAAGAAGAAGCGGAAGGTGGACTCCGGCTGA | |
| Cas9 ORF | ATGGACAAGAAGTACAGCATCGGCCTGGACATCGGCACCAACAGCGTGGGCTGGGCCGTGATCACCGACGAGTACAAGGTGCCCAG | 320 |
| using low | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with two C- | GCACCCCATCTTCGGCAACATCGTGGACGAGGTGGCCTACCACGAGAAGTACCCCACCATCTACCACCTGCGGAAGAAGCTGGTGG | |
| terminal NLS | ACAGCACCGACAAGGCCGACCTGCGGCTGATCTACCTGGCCCTGGCCCACATGATCAAGTTCCGGGGCCACTTCCTGATCGAGGGC | |
| sequences | GACCTGAACCCCGACAACAGCGACGTGGACAAGCTGTTCATCCAGCTGGTGCAGACCTACAACCAGCTGTTCGAGGAGAACCCCAT | |
| and start | CAACGCCAGCGGCGTGGACGCCAAGGCCATCCTGAGCGCCCGGCTGAGCAAGAGCCGGCGGCTGGAGAACCTGATCGCCCAGCTGC | |
| and stop | CCGGCGAGAAGAAGAACGGCCTGTTCGGCAACCTGATCGCCCTGAGCCTGGGCCTGACCCCCAACTTCAAGAGCAACTTCGACCTG | |
| codons | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCAGCGGCAGCCCCAAGAAGAAG | |
| | CGGAAGGTGGACGGCAGCCCCAAGAAGAAGCGGAAGGTGGACAGCGGCTGA | |
| Cas9 ORF | ATGGACAAGAAGTACAGCATCGGCCTGGACATCGGCACCAACAGCGTGGGCTGGGCCGTGATCACCGACGAGTACAAGGTGCCCAG | 321 |
| using low | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACAGCACCGACAAGGCCGACCTGCGGCTGATCTACCTGGCCCTGGCCCACATGATCAAGTTCCGGGGCCACTTCCTGATCGAGGGC | |
| codons and | GACCTGAACCCCGACAACAGCGACGTGGACAAGCTGTTCATCCAGCTGGTGCAGACCTACAACCAGCTGTTCGAGGAGAACCCCAT | |
| no NLS | CAACGCCAGCGGCGTGGACGCCAAGGCCATCCTGAGCGCCCGGCTGAGCAAGAGCCGGCGGCTGGAGAACCTGATCGCCCAGCTGC | |
| | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACTGA | |
| Cas9 nickase | ATGGACAAGAAGTACAGCATCGGCCTGGCCATCGGCACCAACAGCGTGGGCTGGGCCGTGATCACCGACGAGTACAAGGTGCCCAG | 322 |
| ORF using | CAAGAAGTTCAAGGTGCTGGGCAACACCGACCGGCACAGCATCAAGAAGAACCTGATCGGCGCCCTGCTGTTCGACAGCGGCGAGA | |
| low A/U | CCGCCGAGGCCACCCGGCTGAAGCGGACCGCCCGGCGGCGGTACACCCGGCGGAAGAACCGGATCTGCTACCTGCAGGAGATCTTC | |
| codons of | AGCAACGAGATGGCCAAGGTGGACGACAGCTTCTTCCACCGGCTGGAGGAGAGCTTCCTGGTGGAGGAGGACAAGAAGCACGAGCG | |
| Table 4, | GCACCCCATCTTCGGCAACATCGTGGACGAGGTGGCCTACCACGAGAAGTACCCCACCATCTACCACCTGCGGAAGAAGCTGGTGG | |
| with start | ACAGCACCGACAAGGCCGACCTGCGGCTGATCTACCTGGCCCTGGCCCACATGATCAAGTTCCGGGGCCACTTCCTGATCGAGGGC | |
| and stop | GACCTGAACCCCGACAACAGCGACGTGGACAAGCTGTTCATCCAGCTGGTGCAGACCTACAACCAGCTGTTCGAGGAGAACCCCAT | |
| codons | CAACGCCAGCGGCGTGGACGCCAAGGCCATCCTGAGCGCCCGGCTGAGCAAGAGCCGGCGGCTGGAGAACCTGATCGCCCAGCTGC | |
| | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCGGCGGCAGCCCCAAGAAGAAG | |
| | CGGAAGGTGTGA | |
| Cas9 nickase | ATGGACAAGAAGTACAGCATCGGCCTGGCCATCGGCACCAACAGCGTGGGCTGGGCCGTGATCACCGACGAGTACAAGGTGCCCAG | 323 |
| ORF using | CAAGAAGTTCAAGGTGCTGGGCAACACCGACCGGCACAGCATCAAGAAGAACCTGATCGGCGCCCTGCTGTTCGACAGCGGCGAGA | |
| low A/U | CCGCCGAGGCCACCCGGCTGAAGCGGACCGCCCGGCGGCGGTACACCCGGCGGAAGAACCGGATCTGCTACCTGCAGGAGATCTTC | |
| codons of | AGCAACGAGATGGCCAAGGTGGACGACAGCTTCTTCCACCGGCTGGAGGAGAGCTTCCTGGTGGAGGAGGACAAGAAGCACGAGCG | |
| Table 4, | GCACCCCATCTTCGGCAACATCGTGGACGAGGTGGCCTACCACGAGAAGTACCCCACCATCTACCACCTGCGGAAGAAGCTGGTGG | |
| with two C- | ACAGCACCGACAAGGCCGACCTGCGGCTGATCTACCTGGCCCTGGCCCACATGATCAAGTTCCGGGGCCACTTCCTGATCGAGGGC | |
| terminal NLS | GACCTGAACCCCGACAACAGCGACGTGGACAAGCTGTTCATCCAGCTGGTGCAGACCTACAACCAGCTGTTCGAGGAGAACCCCAT | |
| sequences | CAACGCCAGCGGCGTGGACGCCAAGGCCATCCTGAGCGCCCGGCTGAGCAAGAGCCGGCGGCTGGAGAACCTGATCGCCCAGCTGC | |
| and start | CCGGCGAGAAGAAGAACGGCCTGTTCGGCAACCTGATCGCCCTGAGCCTGGGCCTGACCCCCAACTTCAAGAGCAACTTCGACCTG | |
| and stop | GCCGAGGACGCCAAGCTGCAGCTGAGCAAGGACACCTACGACGACGACCTGGACAACCTGCTGGCCCAGATCGGCGACCAGTACGC | |
| codons | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCAGCGGCAGCCCCAAGAAGAAG | |
| | CGGAAGGTGGACGGCAGCCCCAAGAAGAAGCGGAAGGTGGACAGCGGCTGA | |
| Cas9 nickase | ATGGACAAGAAGTACAGCATCGGCCTGGcCATCGGCACCAACAGCGTGGGCTGGGCCGTGATCACCGACGAGTACAAGGTGCCCAG | 324 |
| ORF using | CAAGAAGTTCAAGGTGCTGGGCAACACCGACCGGCACAGCATCAAGAAGAACCTGATCGGCGCCCTGCTGTTCGACAGCGGCGAGA | |
| low A/U | CCGCCGAGGCCACCCGGCTGAAGCGGACCGCCCGGCGGCGGTACACCCGGCGGAAGAACCGGATCTGCTACCTGCAGGAGATCTTC | |
| codons of | AGCAACGAGATGGCCAAGGTGGACGACAGCTTCTTCCACCGGCTGGAGGAGAGCTTCCTGGTGGAGGAGGACAAGAAGCACGAGCG | |
| Table 4, | GCACCCCATCTTCGGCAACATCGTGGACGAGGTGGCCTACCACGAGAAGTACCCCACCATCTACCACCTGCGGAAGAAGCTGGTGG | |
| with start | ACAGCACCGACAAGGCCGACCTGCGGCTGATCTACCTGGCCCTGGCCCACATGATCAAGTTCCGGGGCCACTTCCTGATCGAGGGC | |
| and stop | GACCTGAACCCCGACAACAGCGACGTGGACAAGCTGTTCATCCAGCTGGTGCAGACCTACAACCAGCTGTTCGAGGAGAACCCCAT | |
| codons and | CAACGCCAGCGGCGTGGACGCCAAGGCCATCCTGAGCGCCCGGCTGAGCAAGAGCCGGCGGCTGGAGAACCTGATCGCCCAGCTGC | |
| no NLS | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACCACATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACTGA | |
| dCas9 ORF | ATGGACAAGAAGTACAGCATCGGCCTGGcCATCGGCACCAACAGCGTGGGCTGGGCCGTGATCACCGACGAGTACAAGGTGCCCAG | 325 |
| using | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACAGCACCGACAAGGCCGACCTGCGGCTGATCTACCTGGCCCTGGCCCACATGATCAAGTTCCGGGGCCACTTCCTGATCGAGGGC | |
| codons | GACCTGAACCCCGACAACAGCGACGTGGACAAGCTGTTCATCCAGCTGGTGCAGACCTACAACCAGCTGTTCGAGGAGAACCCCAT | |
| | CAACGCCAGCGGCGTGGACGCCAAGGCCATCCTGAGCGCCCGGCTGAGCAAGAGCCGGCGGCTGGAGAACCTGATCGCCCAGCTGC | |
| | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACgcCATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCGGCGGCAGCCCCAAGAAGAAG | |
| | CGGAAGGTGTGA | |
| dCas9 ORF | ATGGACAAGAAGTACAGCATCGGCCTGGCCATCGGCACCAACAGCGTGGGCTGGGCCGTGATCACCGACGAGTACAAGGTGCCCAG | 326 |
| using low | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with two C- | GCACCCCATCTTCGGCAACATCGTGGACGAGGTGGCCTACCACGAGAAGTACCCCACCATCTACCACCTGCGGAAGAAGCTGGTGG | |
| terminal NLS | ACAGCACCGACAAGGCCGACCTGCGGCTGATCTACCTGGCCCTGGCCCACATGATCAAGTTCCGGGGCCACTTCCTGATCGAGGGC | |
| sequences | GACCTGAACCCCGACAACAGCGACGTGGACAAGCTGTTCATCCAGCTGGTGCAGACCTACAACCAGCTGTTCGAGGAGAACCCCAT | |
| and start | CAACGCCAGCGGCGTGGACGCCAAGGCCATCCTGAGCGCCCGGCTGAGCAAGAGCCGGCGGCTGGAGAACCTGATCGCCCAGCTGC | |
| and stop | CCGGCGAGAAGAAGAACGGCCTGTTCGGCAACCTGATCGCCCTGAGCCTGGGCCTGACCCCCAACTTCAAGAGCAACTTCGACCTG | |
| codons | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACGCCATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACGGCAGCGGCAGCCCCAAGAAGAAG | |
| | CGGAAGGTGGACGGCAGCCCCAAGAAGAAGCGGAAGGTGGACAGCGGCTGA | |
| dCas9 ORF | ATGGACAAGAAGTACAGCATCGGCCTGGcCATCGGCACCAACAGCGTGGGCTGGGCCGTGATCACCGACGAGTACAAGGTGCCCAG | 327 |
| using low | CAAGAAGTTCAAGGTGCTGGGCAACACCGACCGGCACAGCATCAAGAAGAACCTGATCGGCGCCCTGCTGTTCGACAGCGGCGAGA | |
| A/U codons | CCGCCGAGGCCACCCGGCTGAAGCGGACCGCCCGGCGGCGGTACACCCGGCGGAAGAACCGGATCTGCTACCTGCAGGAGATCTTC | |
| of Table 4, | AGCAACGAGATGGCCAAGGTGGACGACAGCTTCTTCCACCGGCTGGAGGAGAGCTTCCTGGTGGAGGAGGACAAGAAGCACGAGCG | |
| with start | GCACCCCATCTTCGGCAACATCGTGGACGAGGTGGCCTACCACGAGAAGTACCCCACCATCTACCACCTGCGGAAGAAGCTGGTGG | |
| and stop | ACAGCACCGACAAGGCCGACCTGCGGCTGATCTACCTGGCCCTGGCCCACATGATCAAGTTCCGGGGCCACTTCCTGATCGAGGGC | |
| codons and | GACCTGAACCCCGACAACAGCGACGTGGACAAGCTGTTCATCCAGCTGGTGCAGACCTACAACCAGCTGTTCGAGGAGAACCCCAT | |
| no NLS | CAACGCCAGCGGCGTGGACGCCAAGGCCATCCTGAGCGCCCGGCTGAGCAAGAGCCGGCGGCTGGAGAACCTGATCGCCCAGCTGC | |
| | CCGGCGAGAAGAAGAACGGCCTGTTCGGCAACCTGATCGCCCTGAGCCTGGGCCTGACCCCCAACTTCAAGAGCAACTTCGACCTG | |
| | GCCGAGGACGCCAAGCTGCAGCTGAGCAAGGACACCTACGACGACGACCTGGACAACCTGCTGGCCCAGATCGGCGACCAGTACGC | |
| | CGACCTGTTCCTGGCCGCCAAGAACCTGAGCGACGCCATCCTGCTGAGCGACATCCTGCGGGTGAACACCGAGATCACCAAGGCCC | |
| | CCCTGAGCGCCAGCATGATCAAGCGGTACGACGAGCACCACCAGGACCTGACCCTGCTGAAGGCCCTGGTGCGGCAGCAGCTGCCC | |
| | GAGAAGTACAAGGAGATCTTCTTCGACCAGAGCAAGAACGGCTACGCCGGCTACATCGACGGCGGCGCCAGCCAGGAGGAGTTCTA | |
| | CAAGTTCATCAAGCCCATCCTGGAGAAGATGGACGGCACCGAGGAGCTGCTGGTGAAGCTGAACCGGGAGGACCTGCTGCGGAAGC | |
| | AGCGGACCTTCGACAACGGCAGCATCCCCCACCAGATCCACCTGGGCGAGCTGCACGCCATCCTGCGGCGGCAGGAGGACTTCTAC | |
| | CCCTTCCTGAAGGACAACCGGGAGAAGATCGAGAAGATCCTGACCTTCCGGATCCCCTACTACGTGGGCCCCCTGGCCCGGGGCAA | |
| | CAGCCGGTTCGCCTGGATGACCCGGAAGAGCGAGGAGACCATCACCCCCTGGAACTTCGAGGAGGTGGTGGACAAGGGCGCCAGCG | |
| | CCCAGAGCTTCATCGAGCGGATGACCAACTTCGACAAGAACCTGCCCAACGAGAAGGTGCTGCCCAAGCACAGCCTGCTGTACGAG | |
| | TACTTCACCGTGTACAACGAGCTGACCAAGGTGAAGTACGTGACCGAGGGCATGCGGAAGCCCGCCTTCCTGAGCGGCGAGCAGAA | |
| | GAAGGCCATCGTGGACCTGCTGTTCAAGACCAACCGGAAGGTGACCGTGAAGCAGCTGAAGGAGGACTACTTCAAGAAGATCGAGT | |
| | GCTTCGACAGCGTGGAGATCAGCGGCGTGGAGGACCGGTTCAACGCCAGCCTGGGCACCTACCACGACCTGCTGAAGATCATCAAG | |
| | GACAAGGACTTCCTGGACAACGAGGAGAACGAGGACATCCTGGAGGACATCGTGCTGACCCTGACCCTGTTCGAGGACCGGGAGAT | |
| | GATCGAGGAGCGGCTGAAGACCTACGCCCACCTGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGACCATCCTGGACTTCCTGAAGAGCGACGGCTTC | |
| | GCCAACCGGAACTTCATGCAGCTGATCCACGACGACAGCCTGACCTTCAAGGAGGACATCCAGAAGGCCCAGGTGAGCGGCCAGGG | |
| | CGACAGCCTGCACGAGCACATCGCCAACCTGGCCGGCAGCCCCGCCATCAAGAAGGGCATCCTGCAGACCGTGAAGGTGGTGGACG | |
| | AGCTGGTGAAGGTGATGGGCCGGCACAAGCCCGAGAACATCGTGATCGAGATGGCCCGGGAGAACCAGACCACCCAGAAGGGCCAG | |
| | AAGAACAGCCGGGAGCGGATGAAGCGGATCGAGGAGGGCATCAAGGAGCTGGGCAGCCAGATCCTGAAGGAGCACCCCGTGGAGAA | |
| | CACCCAGCTGCAGAACGAGAAGCTGTACCTGTACTACCTGCAGAACGGCCGGGACATGTACGTGGACCAGGAGCTGGACATCAACC | |
| | GGCTGAGCGACTACGACGTGGACgcCATCGTGCCCCAGAGCTTCCTGAAGGACGACAGCATCGACAACAAGGTGCTGACCCGGAGC | |
| | GACAAGAACCGGGGCAAGAGCGACAACGTGCCCAGCGAGGAGGTGGTGAAGAAGATGAAGAACTACTGGCGGCAGCTGCTGAACGC | |
| | CAAGCTGATCACCCAGCGGAAGTTCGACAACCTGACCAAGGCCGAGCGGGGCGGCCTGAGCGAGCTGGACAAGGCCGGCTTCATCA | |
| | AGCGGCAGCTGGTGGAGACCCGGCAGATCACCAAGCACGTGGCCCAGATCCTGGACAGCCGGATGAACACCAAGTACGACGAGAAC | |
| | GACAAGCTGATCCGGGAGGTGAAGGTGATCACCCTGAAGAGCAAGCTGGTGAGCGACTTCCGGAAGGACTTCCAGTTCTACAAGGT | |
| | GCGGGAGATCAACAACTACCACCACGCCCACGACGCCTACCTGAACGCCGTGGTGGGCACCGCCCTGATCAAGAAGTACCCCAAGC | |
| | TGGAGAGCGAGTTCGTGTACGGCGACTACAAGGTGTACGACGTGCGGAAGATGATCGCCAAGAGCGAGCAGGAGATCGGCAAGGCC | |
| | ACCGCCAAGTACTTCTTCTACAGCAACATCATGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGACTTCGCCACCGTGCGGAAGGTGCTGAGCATGC | |
| | CCCAGGTGAACATCGTGAAGAAGACCGAGGTGCAGACCGGCGGCTTCAGCAAGGAGAGCATCCTGCCCAAGCGGAACAGCGACAAG | |
| | CTGATCGCCCGGAAGAAGGACTGGGACCCCAAGAAGTACGGCGGCTTCGACAGCCCCACCGTGGCCTACAGCGTGCTGGTGGTGGC | |
| | CAAGGTGGAGAAGGGCAAGAGCAAGAAGCTGAAGAGCGTGAAGGAGCTGCTGGGCATCACCATCATGGAGCGGAGCAGCTTCGAGA | |
| | AGAACCCCATCGACTTCCTGGAGGCCAAGGGCTACAAGGAGGTGAAGAAGGACCTGATCATCAAGCTGCCCAAGTACAGCCTGTTC | |
| | GAGCTGGAGAACGGCCGGAAGCGGATGCTGGCCAGCGCCGGCGAGCTGCAGAAGGGCAACGAGCTGGCCCTGCCCAGCAAGTACGT | |
| | GAACTTCCTGTACCTGGCCAGCCACTACGAGAAGCTGAAGGGCAGCCCCGAGGACAACGAGCAGAAGCAGCTGTTCGTGGAGCAGC | |
| | ACAAGCACTACCTGGACGAGATCATCGAGCAGATCAGCGAGTTCAGCAAGCGGGTGATCCTGGCCGACGCCAACCTGGACAAGGTG | |
| | CTGAGCGCCTACAACAAGCACCGGGACAAGCCCATCCGGGAGCAGGCCGAGAACATCATCCACCTGTTCACCCTGACCAACCTGGG | |
| | CGCCCCCGCCGCCTTCAAGTACTTCGACACCACCATCGACCGGAAGCGGTACACCAGCACCAAGGAGGTGCTGGACGCCACCCTGA | |
| | TCCACCAGAGCATCACCGGCCTGTACGAGACCCGGATCGACCTGAGCCAGCTGGGCGGCGACTGA | |
| Nme Cas9 ORF | ATGGCCGCCTTCAAGCCCAACTCCATCAACTACATCCTGGGCCTGGACATCGGCATCGCCTCCGTGGGCTGGGCCATGGTGGAGAT | 328 |
| using low A | CGACGAGGAGGAGAACCCCATCCGGCTGATCGACCTGGGCGTGCGGGTGTTCGAGCGGGCCGAGGTGCCCAAGACCGGCGACTCCC | |
| codons of | TGGCCATGGCCCGGCGGCTGGCCCGGTCCGTGCGGCGGCTGACCCGGCGGCGGGCCCACCGGCTGCTGCGGACCCGGCGGCTGCTG | |
| Table 4, | AAGCGGGAGGGCGTGCTGCAGGCCGCCAACTTCGACGAGAACGGCCTGATCAAGTCCCTGCCCAACACCCCCTGGCAGCTGCGGGC | |
| with start | CGCCGCCCTGGACCGGAAGCTGACCCCCCTGGAGTGGTCCGCCGTGCTGCTGCACCTGATCAAGCACCGGGGCTACCTGTCCCAGC | |
| and stop | GGAAGAACGAGGGCGAGACCGCCGACAAGGAGCTGGGCGCCCTGCTGAAGGGCGTGGCCGGCAACGCCCACGCCCTGCAGACCGGC | |
| codons | GACTTCCGGACCCCCGCCGAGCTGGCCCTGAACAAGTTCGAGAAGGAGTCCGGCCACATCCGGAACCAGCGGTCCGACTACTCCCA | |
| | CACCTTCTCCCGGAAGGACCTGCAGGCCGAGCTGATCCTGCTGTTCGAGAAGCAGAAGGAGTTCGGCAACCCCCACGTGTCCGGCG | |
| | GCCTGAAGGAGGGCATCGAGACCCTGCTGATGACCCAGCGGCCCGCCCTGTCCGGCGACGCCGTGCAGAAGATGCTGGGCCACTGC | |
| | ACCTTCGAGCCCGCCGAGCCCAAGGCCGCCAAGAACACCTACACCGCCGAGCGGTTCATCTGGCTGACCAAGCTGAACAACCTGCG | |
| | GATCCTGGAGCAGGGCTCCGAGCGGCCCCTGACCGACACCGAGCGGGCCACCCTGATGGACGAGCCCTACCGGAAGTCCAAGCTGA | |
| | CCTACGCCCAGGCCCGGAAGCTGCTGGGCCTGGAGGACACCGCCTTCTTCAAGGGCCTGCGGTACGGCAAGGACAACGCCGAGGCC | |
| | TCCACCCTGATGGAGATGAAGGCCTACCACGCCATCTCCCGGGCCCTGGAGAAGGAGGGCCTGAAGGACAAGAAGTCCCCCCTGAA | |
| | CCTGTCCCCCGAGCTGCAGGACGAGATCGGCACCGCCTTCTCCCTGTTCAAGACCGACGAGGACATCACCGGCCGGCTGAAGGACC | |
| | GGATCCAGCCCGAGATCCTGGAGGCCCTGCTGAAGCACATCTCCTTCGACAAGTTCGTGCAGATCTCCCTGAAGGCCCTGCGGCGG | |
| | ATCGTGCCCCTGATGGAGCAGGGCAAGCGGTACGACGAGGCCTGCGCCGAGATCTACGGCGACCACTACGGCAAGAAGAACACCGA | |
| | GGAGAAGATCTACCTGCCCCCCATCCCCGCCGACGAGATCCGGAACCCCGTGGTGCTGCGGGCCCTGTCCCAGGCCCGGAAGGTGA | |
| | TCAACGGCGTGGTGCGGCGGTACGGCTCCCCCGCCCGGATCCACATCGAGACCGCCCGGGAGGTGGGCAAGTCCTTCAAGGACCGG | |
| | AAGGAGATCGAGAAGCGGCAGGAGGAGAACCGGAAGGACCGGGAGAAGGCCGCCGCCAAGTTCCGGGAGTACTTCCCCAACTTCGT | |
| | GGGCGAGCCCAAGTCCAAGGACATCCTGAAGCTGCGGCTGTACGAGCAGCAGCACGGCAAGTGCCTGTACTCCGGCAAGGAGATCA | |
| | ACCTGGGCCGGCTGAACGAGAAGGGCTACGTGGAGATCGACCACGCCCTGCCCTTCTCCCGGACCTGGGACGACTCCTTCAACAAC | |
| | AAGGTGCTGGTGCTGGGCTCCGAGAACCAGAACAAGGGCAACCAGACCCCCTACGAGTACTTCAACGGCAAGGACAACTCCCGGGA | |
| | GTGGCAGGAGTTCAAGGCCCGGGTGGAGACCTCCCGGTTCCCCCGGTCCAAGAAGCAGCGGATCCTGCTGCAGAAGTTCGACGAGG | |
| | ACGGCTTCAAGGAGCGGAACCTGAACGACACCCGGTACGTGAACCGGTTCCTGTGCCAGTTCGTGGCCGACCGGATGCGGCTGACC | |
| | GGCAAGGGCAAGAAGCGGGTGTTCGCCTCCAACGGCCAGATCACCAACCTGCTGCGGGGCTTCTGGGGCCTGCGGAAGGTGCGGGC | |
| | CGAGAACGACCGGCACCACGCCCTGGACGCCGTGGTGGTGGCCTGCTCCACCGTGGCCATGCAGCAGAAGATCACCCGGTTCGTGC | |
| | GGTACAAGGAGATGAACGCCTTCGACGGCAAGACCATCGACAAGGAGACCGGCGAGGTGCTGCACCAGAAGACCCACTTCCCCCAG | |
| | CCCTGGGAGTTCTTCGCCCAGGAGGTGATGATCCGGGTGTTCGGCAAGCCCGACGGCAAGCCCGAGTTCGAGGAGGCCGACACCCT | |
| | GGAGAAGCTGCGGACCCTGCTGGCCGAGAAGCTGTCCTCCCGGCCCGAGGCCGTGCACGAGTACGTGACCCCCCTGTTCGTGTCCC | |
| | GGGCCCCCAACCGGAAGATGTCCGGCCAGGGCCACATGGAGACCGTGAAGTCCGCCAAGCGGCTGGACGAGGGCGTGTCCGTGCTG | |
| | CGGGTGCCCCTGACCCAGCTGAAGCTGAAGGACCTGGAGAAGATGGTGAACCGGGAGCGGGAGCCCAAGCTGTACGAGGCCCTGAA | |
| | GGCCCGGCTGGAGGCCCACAAGGACGACCCCGCCAAGGCCTTCGCCGAGCCCTTCTACAAGTACGACAAGGCCGGCAACCGGACCC | |
| | AGCAGGTGAAGGCCGTGCGGGTGGAGCAGGTGCAGAAGACCGGCGTGTGGGTGCGGAACCACAACGGCATCGCCGACAACGCCACC | |
| | ATGGTGCGGGTGGACGTGTTCGAGAAGGGCGACAAGTACTACCTGGTGCCCATCTACTCCTGGCAGGTGGCCAAGGGCATCCTGCC | |
| | CGACCGGGCCGTGGTGCAGGGCAAGGACGAGGAGGACTGGCAGCTGATCGACGACTCCTTCAACTTCAAGTTCTCCCTGCACCCCA | |
| | ACGACCTGGTGGAGGTGATCACCAAGAAGGCCCGGATGTTCGGCTACTTCGCCTCCTGCCACCGGGGCACCGGCAACATCAACATC | |
| | CGGATCCACGACCTGGACCACAAGATCGGCAAGAACGGCATCCTGGAGGGCATCGGCGTGAAGACCGCCCTGTCCTTCCAGAAGTA | |
| | CCAGATCGACGAGCTGGGCAAGGAGATCCGGCCCTGCCGGCTGAAGAAGCGGCCCCCCGTGCGGTCCGGCAAGCGGACCGCCGACG | |
| | GCTCCGAGTTCGAGTCCCCCAAGAAGAAGCGGAAGGTGGAGTGA | |
| Nme Cas9 ORF | ATGGCCGCCTTCAAGCCCAACAGCATCAACTACATCCTGGGCCTGGACATCGGCATCGCCAGCGTGGGCTGGGCCATGGTGGAGAT | 329 |
| using low | CGACGAGGAGGAGAACCCCATCCGGCTGATCGACCTGGGCGTGCGGGTGTTCGAGCGGGCCGAGGTGCCCAAGACCGGCGACAGCC | |
| A/U codons | TGGCCATGGCCCGGCGGCTGGCCCGGAGCGTGCGGCGGCTGACCCGGCGGCGGGCCCACCGGCTGCTGCGGACCCGGCGGCTGCTG | |
| of Table 4, | AAGCGGGAGGGCGTGCTGCAGGCCGCCAACTTCGACGAGAACGGCCTGATCAAGAGCCTGCCCAACACCCCCTGGCAGCTGCGGGC | |
| with start | CGCCGCCCTGGACCGGAAGCTGACCCCCCTGGAGTGGAGCGCCGTGCTGCTGCACCTGATCAAGCACCGGGGCTACCTGAGCCAGC | |
| and stop | GGAAGAACGAGGGCGAGACCGCCGACAAGGAGCTGGGCGCCCTGCTGAAGGGCGTGGCCGGCAACGCCCACGCCCTGCAGACCGGC | |
| codons | GACTTCCGGACCCCCGCCGAGCTGGCCCTGAACAAGTTCGAGAAGGAGAGCGGCCACATCCGGAACCAGCGGAGCGACTACAGCCA | |
| | CACCTTCAGCCGGAAGGACCTGCAGGCCGAGCTGATCCTGCTGTTCGAGAAGCAGAAGGAGTTCGGCAACCCCCACGTGAGCGGCG | |
| | GCCTGAAGGAGGGCATCGAGACCCTGCTGATGACCCAGCGGCCCGCCCTGAGCGGCGACGCCGTGCAGAAGATGCTGGGCCACTGC | |
| | ACCTTCGAGCCCGCCGAGCCCAAGGCCGCCAAGAACACCTACACCGCCGAGCGGTTCATCTGGCTGACCAAGCTGAACAACCTGCG | |
| | GATCCTGGAGCAGGGCAGCGAGCGGCCCCTGACCGACACCGAGCGGGCCACCCTGATGGACGAGCCCTACCGGAAGAGCAAGCTGA | |
| | CCTACGCCCAGGCCCGGAAGCTGCTGGGCCTGGAGGACACCGCCTTCTTCAAGGGCCTGCGGTACGGCAAGGACAACGCCGAGGCC | |
| | AGCACCCTGATGGAGATGAAGGCCTACCACGCCATCAGCCGGGCCCTGGAGAAGGAGGGCCTGAAGGACAAGAAGAGCCCCCTGAA | |
| | CCTGAGCCCCGAGCTGCAGGACGAGATCGGCACCGCCTTCAGCCTGTTCAAGACCGACGAGGACATCACCGGCCGGCTGAAGGACC | |
| | GGATCCAGCCCGAGATCCTGGAGGCCCTGCTGAAGCACATCAGCTTCGACAAGTTCGTGCAGATCAGCCTGAAGGCCCTGCGGCGG | |
| | ATCGTGCCCCTGATGGAGCAGGGCAAGCGGTACGACGAGGCCTGCGCCGAGATCTACGGCGACCACTACGGCAAGAAGAACACCGA | |
| | GGAGAAGATCTACCTGCCCCCCATCCCCGCCGACGAGATCCGGAACCCCGTGGTGCTGCGGGCCCTGAGCCAGGCCCGGAAGGTGA | |
| | TCAACGGCGTGGTGCGGCGGTACGGCAGCCCCGCCCGGATCCACATCGAGACCGCCCGGGAGGTGGGCAAGAGCTTCAAGGACCGG | |
| | AAGGAGATCGAGAAGCGGCAGGAGGAGAACCGGAAGGACCGGGAGAAGGCCGCCGCCAAGTTCCGGGAGTACTTCCCCAACTTCGT | |
| | GGGCGAGCCCAAGAGCAAGGACATCCTGAAGCTGCGGCTGTACGAGCAGCAGCACGGCAAGTGCCTGTACAGCGGCAAGGAGATCA | |
| | ACCTGGGCCGGCTGAACGAGAAGGGCTACGTGGAGATCGACCACGCCCTGCCCTTCAGCCGGACCTGGGACGACAGCTTCAACAAC | |
| | AAGGTGCTGGTGCTGGGCAGCGAGAACCAGAACAAGGGCAACCAGACCCCCTACGAGTACTTCAACGGCAAGGACAACAGCCGGGA | |
| | GTGGCAGGAGTTCAAGGCCCGGGTGGAGACCAGCCGGTTCCCCCGGAGCAAGAAGCAGCGGATCCTGCTGCAGAAGTTCGACGAGG | |
| | ACGGCTTCAAGGAGCGGAACCTGAACGACACCCGGTACGTGAACCGGTTCCTGTGCCAGTTCGTGGCCGACCGGATGCGGCTGACC | |
| | GGCAAGGGCAAGAAGCGGGTGTTCGCCAGCAACGGCCAGATCACCAACCTGCTGCGGGGCTTCTGGGGCCTGCGGAAGGTGCGGGC | |
| | CGAGAACGACCGGCACCACGCCCTGGACGCCGTGGTGGTGGCCTGCAGCACCGTGGCCATGCAGCAGAAGATCACCCGGTTCGTGC | |
| | GGTACAAGGAGATGAACGCCTTCGACGGCAAGACCATCGACAAGGAGACCGGCGAGGTGCTGCACCAGAAGACCCACTTCCCCCAG | |
| | CCCTGGGAGTTCTTCGCCCAGGAGGTGATGATCCGGGTGTTCGGCAAGCCCGACGGCAAGCCCGAGTTCGAGGAGGCCGACACCCT | |
| | GGAGAAGCTGCGGACCCTGCTGGCCGAGAAGCTGAGCAGCCGGCCCGAGGCCGTGCACGAGTACGTGACCCCCCTGTTCGTGAGCC | |
| | GGGCCCCCAACCGGAAGATGAGCGGCCAGGGCCACATGGAGACCGTGAAGAGCGCCAAGCGGCTGGACGAGGGCGTGAGCGTGCTG | |
| | CGGGTGCCCCTGACCCAGCTGAAGCTGAAGGACCTGGAGAAGATGGTGAACCGGGAGCGGGAGCCCAAGCTGTACGAGGCCCTGAA | |
| | GGCCCGGCTGGAGGCCCACAAGGACGACCCCGCCAAGGCCTTCGCCGAGCCCTTCTACAAGTACGACAAGGCCGGCAACCGGACCC | |
| | AGCAGGTGAAGGCCGTGCGGGTGGAGCAGGTGCAGAAGACCGGCGTGTGGGTGCGGAACCACAACGGCATCGCCGACAACGCCACC | |
| | ATGGTGCGGGTGGACGTGTTCGAGAAGGGCGACAAGTACTACCTGGTGCCCATCTACAGCTGGCAGGTGGCCAAGGGCATCCTGCC | |
| | CGACCGGGCCGTGGTGCAGGGCAAGGACGAGGAGGACTGGCAGCTGATCGACGACAGCTTCAACTTCAAGTTCAGCCTGCACCCCA | |
| | ACGACCTGGTGGAGGTGATCACCAAGAAGGCCCGGATGTTCGGCTACTTCGCCAGCTGCCACCGGGGCACCGGCAACATCAACATC | |
| | CGGATCCACGACCTGGACCACAAGATCGGCAAGAACGGCATCCTGGAGGGCATCGGCGTGAAGACCGCCCTGAGCTTCCAGAAGTA | |
| | CCAGATCGACGAGCTGGGCAAGGAGATCCGGCCCTGCCGGCTGAAGAAGCGGCCCCCCGTGCGGAGCGGCAAGCGGACCGCCGACG | |
| | GCAGCGAGTTCGAGAGCCCCAAGAAGAAGCGGAAGGTGGAGTGA | |
| Open reading | ATGGACAAGAAGTACAGCATCGGACTGGACATCGGAACAAACAGCGTCGGATGGGCAGTCATCACAGACGAATACAAGGTCCCGAG | 330 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS1, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCTGGCAGCAAAG | |
| | AGAAGCAGAACAACATAG | |
| Open reading | ATGGACAAGAAGTACAGCATCGGACTGGACATCGGAACAAACAGCGTCGGATGGGCAGTCATCACAGACGAATACAAGGTCCCGAG | 331 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS2, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGPAAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCAGGCAGCAAAG | |
| | AGAAGCAGAACAACATAG | |
| Open reading | ATGGACAAGAAGTACAGCATCGGACTGGACATCGGAACAAACAGCGTCGGATGGGCAGTCATCACAGACGAATACAAGGTCCCGAG | 332 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS 3, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCCGGCACCGGCA | |
| | AAGAGAGAAAGAACAACATAG | |
| Open reading | ATGGACAAGAAGTACAGCATCGGACTGGACATCGGAACAAACAGCGTCGGATGGGCAGTCATCACAGACGAATACAAGGTCCCGAG | 333 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS4, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCG+AGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCCAGGCAGCAAAG | |
| | AGACCGAGAACAACATAG | |
| Open reading | ATGGACAAGAAGTACAGCATCGGACTGGACATCGGAACAAACAGCGTCGGATGGGCAGTCATCACAGACGAATACAAGGTCCCGAG | 334 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS 5, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCAGAGCAGCAAAG | |
| | AGACCGAGAACAACATAG | |
| Open reading | ATGGACAAGAAGTACAGCATCGGACTGGACATCGGAACAAACAGCGTCGGATGGGCAGTCATCACAGACGAATACAAGGTCCCGAG | 335 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS 6, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCG+AGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCGCAGCAGCAAAG | |
| | AGAAGCTGGAGCATGGCAGCATAG | |
| Open reading | ATGGACAAGAAGTACAGCATCGGACTGGACATCGGAACAAACAGCGTCGGATGGGCAGTCATCACAGACGAATACAAGGTCCCGAG | 336 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS7, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCGCAGCAGCAAAG | |
| | AGAGTCTGGAGCATGGCATTCTAG | |
| Open reading | ATGGACAAGAAGTACAGCATCGGACTGGACATCGGAACAAACAGCGTCGGATGGGCAGTCATCACAGACGAATACAAGGTCCCGAG | 337 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS 8, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCGCAGCAGCAAAG | |
| | AGAAGCTGGAGCATGGCATTCTAG | |
| Open reading | ATGGACAAGAAGTACAGCATCGGACTGGACATCGGAACAAACAGCGTCGGATGGGCAGTCATCACAGACGAATACAAGGTCCCGAG | 338 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS9, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCG+AGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCGCAGCAGCAAAG | |
| | AGAAAGTACTTCGCAGCATAG | |
| Open reading | ATGGACAAGAAGTACAGCATCGGACTGGACATCGGAACAAACAGCGTCGGATGGGCAGTCATCACAGACGAATACAAGGTCCCGAG | 339 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS10, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGAAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCAGAGCAGCAAAG | |
| | AGAAAGGCATTCGCAGCATAG | |
| Open reading | ATGGACAAGAAGTACAGCATCGGACTGGACATCGGAACAAACAGCGTCGGATGGGCAGTCATCACAGACGAATACAAGGTCCCGAG | 340 |
| frame for | CAAGAAGTTCAAGGTCCTGGGAAACACAGACAGACACAGCATCAAGAAGAACCTGATCGGAGCACTGCTGTTCGACAGCGGAGAAA | |
| Cas9 with | CAGCAGAAGCAACAAGACTGAAGAGAACAGCAAGAAGAAGATACACAAGAAGAAAGAACAGAATCTGCTACCTGCAGGAAATCTTC | |
| NLS11, with | AGCAACGAAATGGCAAAGGTCGACGACAGCTTCTTCCACAGACTGGAAGAAAGCTTCCTGGTCGAAGAAGACAAGAAGCACGAAAG | |
| start and | ACACCCGATCTTCGGAAACATCGTCGACGAAGTCGCATACCACGAAAAGTACCCGACAATCTACCACCTGAGAAAGAAGCTGGTCG | |
| stop codons | ACAGCACAGACAAGGCAGACCTGAGACTGATCTACCTGGCACTGGCACACATGATCAAGTTCAGAGGACACTTCCTGATCGAAGGA | |
| | GACCTGAACCCGGACAACAGCGACGTCGACAAGCTGTTCATCCAGCTGGTCCAGACATACAACCAGCTGTTCGAAGAAAACCCGAT | |
| | CAACGCAAGCGGAGTCGACGCAAAGGCAATCCTGAGCGCAAGACTGAGCAAGAGCAGAAGACTGGAAAACCTGATCGCACAGCTGC | |
| | CGGGAGAAAAGAAGAACGGACTGTTCGGAAACCTGATCGCACTGAGCCTGGGACTGACACCGAACTTCAAGAGCAACTTCGACCTG | |
| | GCAGAAGACGCAAAGCTGCAGCTGAGCAAGGACACATACGACGACGACCTGGACAACCTGCTGGCACAGATCGGAGACCAGTACGC | |
| | AGACCTGTTCCTGGCAGCAAAGAACCTGAGCGACGCAATCCTGCTGAGCGACATCCTGAGAGTCAACACAGAAATCACAAAGGCAC | |
| | CGCTGAGCGCAAGCATGATCAAGAGATACGACGAACACCACCAGGACCTGACACTGCTGAAGGCACTGGTCAGACAGCAGCTGCCG | |
| | GAAAAGTACAAGGAAATCTTCTTCGACCAGAGCAAGAACGGATACGCAGGATACATCGACGGAGGAGCAAGCCAGGAAGAATTCTA | |
| | CAAGTTCATCAAGCCGATCCTGGAAAAGATGGACGGAACAGAAGAACTGCTGGTCAAGCTGAACAGAGAAGACCTGCTGAGAAAGC | |
| | AGAGAACATTCGACAACGGAAGCATCCCGCACCAGATCCACCTGGGAGAACTGCACGCAATCCTGAGAAGACAGGAAGACTTCTAC | |
| | CCGTTCCTGAAGGACAACAGAGAAAAGATCGAAAAGATCCTGACATTCAGAATCCCGTACTACGTCGGACCGCTGGCAAGAGGAAA | |
| | CAGCAGATTCGCATGGATGACAAGAAAGAGCGAAGAAACAATCACACCGTGGAACTTCGAAGAAGTCGTCGACAAGGGAGCAAGCG | |
| | CACAGAGCTTCATCGAAAGAATGACAAACTTCGACAAGAACCTGCCGAACGAAAAGGTCCTGCCGAAGCACAGCCTGCTGTACGAA | |
| | TACTTCACAGTCTACAACGAACTGACAAAGGTCAAGTACGTCACAGAAGGAATGAGAAAGCCGGCATTCCTGAGCGGAGAACAGAA | |
| | GAAGGCAATCGTCGACCTGCTGTTCAAGACAAACAGAAAGGTCACAGTCAAGCAGCTGAAGGAAGACTACTTCAAGAAGATCGAAT | |
| | GCTTCGACAGCGTCGAAATCAGCGGAGTCGAAGACAGATTCAACGCAAGCCTGGGAACATACCACGACCTGCTGAAGATCATCAAG | |
| | GACAAGGACTTCCTGGACAACGAAGAAAACGAAGACATCCTGGAAGACATCGTCCTGACACTGACACTGTTCGAAGACAGAGAAAT | |
| | GATCGAAGAAAGACTGAAGACATACGCACACCTGTTCGACGACAAGGTCATGAAGCAGCTGAAGAGAAGAAGATACACAGGATGGG | |
| | GAAGACTGAGCAGAAAGCTGATCAACGGAATCAGAGACAAGCAGAGCGGAAAGACAATCCTGGACTTCCTGAAGAGCGACGGATTC | |
| | GCAAACAGAAACTTCATGCAGCTGATCCACGACGACAGCCTGACATTCAAGGAAGACATCCAGAAGGCACAGGTCAGCGGACAGGG | |
| | AGACAGCCTGCACGAACACATCGCAAACCTGGCAGGAAGCCCGGCAATCAAGAAGGGAATCCTGCAGACAGTCAAGGTCGTCGACG | |
| | AACTGGTCAAGGTCATGGGAAGACACAAGCCGGAAAACATCGTCATCGAAATGGCAAGAGAAAACCAGACAACACAGAAGGGACAG | |
| | AAGAACAGCAGAGAAAGAATGAAGAGAATCGAAGAAGGAATCAAGGAACTGGGAAGCCAGATCCTGAAGGAACACCCGGTCGAAAA | |
| | CACACAGCTGCAGAACGAAAAGCTGTACCTGTACTACCTGCAGAACGGAAGAGACATGTACGTCGACCAGGAACTGGACATCAACA | |
| | GACTGAGCGACTACGACGTCGACCACATCGTCCCGCAGAGCTTCCTGAAGGACGACAGCATCGACAACAAGGTCCTGACAAGAAGC | |
| | GACAAGAACAGAGGAAAGAGCGACAACGTCCCGAGCGAAGAAGTCGTCAAGAAGATGAAGAACTACTGGAGACAGCTGCTGAACGC | |
| | AAAGCTGATCACACAGAGAAAGTTCGACAACCTGACAAAGGCAGAGAGAGGAGGACTGAGCGAACTGGACAAGGCAGGATTCATCA | |
| | AGAGACAGCTGGTCGAAACAAGACAGATCACAAAGCACGTCGCACAGATCCTGGACAGCAGAATGAACACAAAGTACGACGAAAAC | |
| | GACAAGCTGATCAGAGAAGTCAAGGTCATCACACTGAAGAGCAAGCTGGTCAGCGACTTCAGAAAGGACTTCCAGTTCTACAAGGT | |
| | CAGAGAAATCAACAACTACCACCACGCACACGACGCATACCTGAACGCAGTCGTCGGAACAGCACTGATCAAGAAGTACCCGAAGC | |
| | TGGAAAGCGAATTCGTCTACGGAGACTACAAGGTCTACGACGTCAGAAAGATGATCGCAAAGAGCGAACAGGAAATCGGAAAGGCA | |
| | ACAGCAAAGTACTTCTTCTACAGCAACATCATGAACTTCTTCAAGACAGAAATCACACTGGCAAACGGAGAAATCAGAAAGAGACC | |
| | GCTGATCGAAACAAACGGAGAAACAGGAGAAATCGTCTGGGACAAGGGAAGAGACTTCGCAACAGTCAGAAAGGTCCTGAGCATGC | |
| | CGCAGGTCAACATCGTCAAGAAGACAGAAGTCCAGACAGGAGGATTCAGCAAGGAAAGCATCCTGCCGAAGAGAAACAGCGACAAG | |
| | CTGATCGCAAGAAAGAAGGACTGGGACCCGAAGAAGTACGGAGGATTCGACAGCCCGACAGTCGCATACAGCGTCCTGGTCGTCGC | |
| | AAAGGTCGAAAAGGGAAAGAGCAAGAAGCTGAAGAGCGTCAAGGAACTGCTGGGAATCACAATCATGGAAAGAAGCAGCTTCGAAA | |
| | AGAACCCGATCGACTTCCTGGAAGCAAAGGGATACAAGGAAGTCAAGAAGGACCTGATCATCAAGCTGCCGAAGTACAGCCTGTTC | |
| | GAACTGGAAAACGGAAGPAAGAGAATGCTGGCAAGCGCAGGAGAACTGCAGAAGGGAAACGAACTGGCACTGCCGAGCAAGTACGT | |
| | CAACTTCCTGTACCTGGCAAGCCACTACGAAAAGCTGAAGGGAAGCCCGGAAGACAACGAACAGAAGCAGCTGTTCGTCGAACAGC | |
| | ACAAGCACTACCTGGACGAAATCATCGAACAGATCAGCGAATTCAGCAAGAGAGTCATCCTGGCAGACGCAAACCTGGACAAGGTC | |
| | CTGAGCGCATACAACAAGCACAGAGACAAGCCGATCAGAGAACAGGCAGAAAACATCATCCACCTGTTCACACTGACAAACCTGGG | |
| | AGCACCGGCAGCATTCAAGTACTTCGACACAACAATCGACAGAAAGAGATACACAAGCACAAAGGAAGTCCTGGACGCAACACTGA | |
| | TCCACCAGAGCATCACAGGACTGTACGAAACAAGAATCGACCTGAGCCAGCTGGGAGGAGACGGAGGAGGAAGCAGAGCAGCAAAG | |
| | AGAAAGTACTTCGCAGTCTAG | |
| Cas9 ORF | CCTAAGAAAAAGCGGAAGGTCGACGGGGATAAGAAGTACTCAATCGGGCTGGATATCGGAACTAATTCCGTGGGTTGGGCAGTGAT | 341 |
| using codons | CACGGATGAATACAAAGTGCCGTCCAAGAAGTTCAAGGTCCTGGGGAACACCGATAGACACAGCATCAAGAAAAATCTCATCGGAG | |
| with | CCCTGCTGTTTGACTCCGGCGAAACCGCAGAAGCGACCCGGCTCAAACGTACCGCGAGGCGACGCTACACCCGGCGGAAGAATCGC | |
| generally | ATCTGCTATCTGCAAGAGATCTTTTCGAACGAAATGGCAAAGGTCGACGACAGCTTCTTCCACCGCCTGGAAGAATCTTTCCTGGT | |
| high | GGAGGAGGACAAGAAGCATGAACGGCATCCTATCTTTGGAAACATCGTCGACGAAGTGGCGTACCACGAAAAGTACCCGACCATCT | |
| expression | ACCATCTGCGGAAGAAGTTGGTTGACTCAACTGACAAGGCCGACCTCAGATTGATCTACTTGGCCCTCGCCCATATGATCAAATTC | |
| in humans | CGCGGACACTTCCTGATCGAAGGCGATCTGAACCCTGATAACTCCGACGTGGATAAGCTTTTCATTCAACTGGTGCAGACCTACAA | |
| (no start or | CCAACTGTTCGAAGAAAACCCAATCAATGCTAGCGGCGTCGATGCCAAGGCCATCCTGTCCGCCCGGCTGTCGAAGTCGCGGCGCC | |
| stop codons; | TCGAAAACCTGATCGCACAGCTGCCGGGAGAGAAAAAGAACGGACTTTTCGGCAACTTGATCGCTCTCTCACTGGGACTCACTCCC | |
| suitable for | AATTTCAAGTCCAATTTTGACCTGGCCGAGGACGCGAAGCTGCAACTCTCAAAGGACACCTACGACGACGACTTGGACAATTTGCT | |
| inclusion in | GGCACAAATTGGCGATCAGTACGCGGATCTGTTCCTTGCCGCTAAGAACCTTTCGGACGCAATCTTGCTGTCCGATATCCTGCGCG | |
| fusion | TGAACACCGAAATAACCAAAGCGCCGCTTAGCGCCTCGATGATTAAGCGGTACGACGAGCATCACCAGGATCTCACGCTGCTCAAA | |
| protein | GCGCTCGTGAGACAGCAACTGCCTGAAAAGTACAAGGAGATCTTCTTCGACCAGTCCAAGAATGGGTACGCAGGGTACATCGATGG | |
| coding | AGGCGCTAGCCAGGAAGAGTTCTATAAGTTCATCAAGCCAATCCTGGAAAAGATGGACGGAACCGAAGAACTGCTGGTCAAGCTGA | |
| sequence) | ACAGGGAGGATCTGCTCCGGAAACAGAGAACCTTTGACAACGGATCCATTCCCCACCAGATCCATCTGGGTGAGCTGCACGCCATC | |
| | TTGCGGCGCCAGGAGGACTTTTACCCATTCCTCAAGGACAACCGGGAAAAGATCGAGAAAATTCTGACGTTCCGCATCCCGTATTA | |
| | CGTGGGCCCACTGGCGCGCGGCAATTCGCGCTTCGCGTGGATGACTAGAAAATCAGAGGAAACCATCACTCCTTGGAATTTCGAGG | |
| | AAGTTGTGGATAAGGGAGCTTCGGCACAAAGCTTCATCGAACGAATGACCAACTTCGACAAGAATCTCCCAAACGAGAAGGTGCTT | |
| | CCTAAGCACAGCCTCCTTTACGAATACTTCACTGTCTACAACGAACTGACTAAAGTGAAATACGTTACTGAAGGAATGAGGAAGCC | |
| | GGCCTTTCTGTCCGGAGAACAGAAGAAAGCAATTGTCGATCTGCTGTTCAAGACCAACCGCAAGGTGACCGTCAAGCAGCTTAAAG | |
| | AGGACTACTTCAAGAAGATCGAGTGTTTCGACTCAGTGGAAATCAGCGGGGTGGAGGACAGATTCAACGCTTCGCTGGGAACCTAT | |
| | CATGATCTCCTGAAGATCATCAAGGACAAGGACTTCCTTGACAACGAGGAGAACGAGGACATCCTGGAAGATATCGTCCTGACCTT | |
| | GACCCTTTTCGAGGATCGCGAGATGATCGAGGAGAGGCTTAAGACCTACGCTCATCTCTTCGACGATAAGGTCATGAAACAACTCA | |
| | AGCGCCGCCGGTACACTGGTTGGGGCCGCCTCTCCCGCAAGCTGATCAACGGTATTCGCGATAAACAGAGCGGTAAAACTATCCTG | |
| | GATTTCCTCAAATCGGATGGCTTCGCTAATCGTAACTTCATGCAATTGATCCACGACGACAGCCTGACCTTTAAGGAGGACATCCA | |
| | AAAAGCACAAGTGTCCGGACAGGGAGACTCACTCCATGAACACATCGCGAATCTGGCCGGTTCGCCGGCGATTAAGAAGGGAATTC | |
| | TGCAAACTGTGAAGGTGGTCGACGAGCTGGTGAAGGTCATGGGACGGCACAAACCGGAGAATATCGTGATTGAAATGGCCCGAGAA | |
| | AACCAGACTACCCAGAAGGGCCAGAAAAACTCCCGCGAAAGGATGAAGCGGATCGAAGAAGGAATCAAGGAGCTGGGCAGCCAGAT | |
| | CCTGAAAGAGCACCCGGTGGAAAACACGCAGCTGCAGAACGAGAAGCTCTACCTGTACTATTTGCAAAATGGACGGGACATGTACG | |
| | TGGACCAAGAGCTGGACATCAATCGGTTGTCTGATTACGACGTGGACCACATCGTTCCACAGTCCTTTCTGAAGGATGACTCGATC | |
| | GATAACAAGGTGTTGACTCGCAGCGACAAGAACAGAGGGAAGTCAGATAATGTGCCATCGGAGGAGGTCGTGAAGAAGATGAAGAA | |
| | TTACTGGCGGCAGCTCCTGAATGCGAAGCTGATTACCCAGAGAAAGTTTGACAATCTCACTAAAGCCGAGCGCGGCGGACTCTCAG | |
| | AGCTGGATAAGGCTGGATTCATCAAACGGCAGCTGGTCGAGACTCGGCAGATTACCAAGCACGTGGCGCAGATCTTGGACTCCCGC | |
| | ATGAACACTAAATACGACGAGAACGATAAGCTCATCCGGGAAGTGAAGGTGATTACCCTGAAAAGCAAACTTGTGTCGGACTTTCG | |
| | GAAGGACTTTCAGTTTTACAAAGTGAGAGAAATCAACAACTACCATCACGCGCATGACGCATACCTCAACGCTGTGGTCGGTACCG | |
| | CCCTGATCAAAAAGTACCCTAAACTTGAATCGGAGTTTGTGTACGGAGACTACAAGGTCTACGACGTGAGGAAGATGATAGCCAAG | |
| | TCCGAACAGGAAATCGGGAAAGCAACTGCGAAATACTTCTTTTACTCAAACATCATGAACTTTTTCAAGACTGAAATTACGCTGGC | |
| | CAATGGAGAAATCAGGAAGAGGCCACTGATCGAAACTAACGGAGAAACGGGCGAAATCGTGTGGGACAAGGGCAGGGACTTCGCAA | |
| | CTGTTCGCAAAGTGCTCTCTATGCCGCAAGTCAATATTGTGAAGAAAACCGAAGTGCAAACCGGCGGATTTTCAAAGGAATCGATC | |
| | CTCCCAAAGAGAAATAGCGACAAGCTCATTGCACGCAAGAAAGACTGGGACCCGAAGAAGTACGGAGGATTCGATTCGCCGACTGT | |
| | CGCATACTCCGTCCTCGTGGTGGCCAAGGTGGAGAAGGGAAAGAGCAAAAAGCTCAAATCCGTCAAAGAGCTGCTGGGGATTACCA | |
| | TCATGGAACGATCCTCGTTCGAGAAGAACCCGATTGATTTCCTCGAGGCGAAGGGTTACAAGGAGGTGAAGAAGGATCTGATCATC | |
| | AAACTCCCCAAGTACTCACTGTTCGAACTGGAAAATGGTCGGAAGCGCATGCTGGCTTCGGCCGGAGAACTCCAAAAAGGAAATGA | |
| | GCTGGCCTTGCCTAGCAAGTACGTCAACTTCCTCTATCTTGCTTCGCACTACGAAAAACTCAAAGGGTCACCGGAAGATAACGAAC | |
| | AGAAGCAGCTTTTCGTGGAGCAGCACAAGCATTATCTGGATGAAATCATCGAACAAATCTCCGAGTTTTCAAAGCGCGTGATCCTC | |
| | GCCGACGCCAACCTCGACAAAGTCCTGTCGGCCTACAATAAGCATAGAGATAAGCCGATCAGAGAACAGGCCGAGAACATTATCCA | |
| | CTTGTTCACCCTGACTAACCTGGGAGCCCCAGCCGCCTTCAAGTACTTCGATACTACTATCGATCGCAAAAGATACACGTCCACCA | |
| | AGGAAGTTCTGGACGCGACCCTGATCCACCAAAGCATCACTGGACTCTACGAAACTAGGATCGATCTGTCGCAGCTGGGTGGCGAT | |
| Cas9 ORF | GACAAGAAGTACTCTATCGGTTTGGACATCGGTACCAACTCTGTCGGTTGGGCCGTCATCACCGACGAATACAAGGTCCCATCTAA | 342 |
| using long | GAAGTTCAAGGTCTTGGGTAACACCGACAGACACTCTATCAAGAAGAACTTGATCGGTGCCTTGTTGTTCGACTCTGGTGAAACCG | |
| half life | CCGAAGCCACCAGATTGAAGAGAACCGCCAGAAGAAGATACACCAGAAGAAAGAACAGAATCTGCTACTTGCAAGAAATCTTCTCT | |
| codons of | AACGAAATGGCCAAGGTCGACGACTCTTTCTTCCACAGATTGGAAGAATCTTTCTTGGTCGAAGAAGACAAGAAGCACGAAAGACA | |
| Table 4 (no | CCCAATCTTCGGTAACATCGTCGACGAAGTCGCCTACCACGAAAAGTACCCAACCATCTACCACTTGAGAAAGAAGTTGGTCGACT | |
| start or | CTACCGACAAGGCCGACTTGAGATTGATCTACTTGGCCTTGGCCCACATGATCAAGTTCAGAGGTCACTTCTTGATCGAAGGTGAC | |
| stop codons; | TTGAACCCAGACAACTCTGACGTCGACAAGTTGTTCATCCAATTGGTCCAAACCTACAACCAATTGTTCGAAGAAAACCCAATCAA | |
| suitable for | CGCCTCTGGTGTCGACGCCAAGGCCATCTTGTCTGCCAGATTGTCTAAGAGCAGAAGATTGGAAAACTTGATCGCCCAATTGCCAG | |
| inclusion in | GTGAAAAGAAGAACGGTTTGTTCGGTAACTTGATCGCCTTGTCTTTGGGTTTGACCCCAAACTTCAAGTCTAACTTCGACTTGGCC | |
| fusion | GAAGACGCCAAGTTGCAATTGTCTAAGGACACCTACGACGACGACTTGGACAACTTGTTGGCCCAAATCGGTGACCAATACGCCGA | |
| protein | CTTGTTCTTGGCCGCCAAGAACTTGTCTGACGCCATCTTGTTGTCTGACATCTTGAGAGTCAACACCGAAATCACCAAGGCCCCAT | |
| coding | TGTCTGCCTCTATGATCAAGAGATACGACGAACACCACCAAGACTTGACCTTGTTGAAGGCCTTGGTCAGACAACAATTGCCAGAA | |
| sequence) | AAGTACAAGGAAATCTTCTTCGACCAATCTAAGAACGGTTACGCCGGTTACATCGACGGTGGTGCCTCTCAAGAAGAATTCTACAA | |
| | GTTCATCAAGCCAATCTTGGAAAAGATGGACGGTACCGAAGAATTGTTGGTCAAGTTGAACAGAGAAGACTTGTTGAGAAAGCAAA | |
| | GAACCTTCGACAACGGTTCTATCCCACACCAAATCCACTTGGGTGAATTGCACGCCATCTTGAGAAGACAAGAAGACTTCTACCCA | |
| | TTCTTGAAGGACAACAGAGAAAAGATCGAAAAGATCTTGACCTTCAGAATCCCATACTACGTCGGTCCATTGGCCAGAGGTAACAG | |
| | CAGATTCGCCTGGATGACCAGAAAGTCTGAAGAAACCATCACCCCATGGAACTTCGAAGAAGTCGTCGACAAGGGTGCCTCTGCCC | |
| | AATCTTTCATCGAAAGAATGACCAACTTCGACAAGAACTTGCCAAACGAAAAGGTCTTGCCAAAGCACTCTTTGTTGTACGAATAC | |
| | TTCACCGTCTACAACGAATTGACCAAGGTCAAGTACGTCACCGAAGGTATGAGAAAGCCAGCCTTCTTGTCTGGTGAACAAAAGAA | |
| | GGCCATCGTCGACTTGTTGTTCAAGACCAACAGAAAGGTCACCGTCAAGCAATTGAAGGAAGACTACTTCAAGAAGATCGAATGCT | |
| | TCGACTCTGTCGAAATCTCTGGTGTCGAAGACAGATTCAACGCCTCTTTGGGTACCTACCACGACTTGTTGAAGATCATCAAGGAC | |
| | AAGGACTTCTTGGACAACGAAGAAAACGAAGACATCTTGGAAGACATCGTCTTGACCTTGACCTTGTTCGAAGACAGAGAAATGAT | |
| | CGAAGAAAGATTGAAGACCTACGCCCACTTGTTCGACGACAAGGTCATGAAGCAATTGAAGAGAAGAAGATACACCGGTTGGGGTA | |
| | GATTGAGCAGAAAGTTGATCAACGGTATCAGAGACAAGCAATCTGGTAAGACCATCTTGGACTTCTTGAAGTCTGACGGTTTCGCC | |
| | AACAGAAACTTCATGCAATTGATCCACGACGACTCTTTGACCTTCAAGGAAGACATCCAAAAGGCCCAAGTCTCTGGTCAAGGTGA | |
| | CTCTTTGCACGAACACATCGCCAACTTGGCCGGTTCTCCAGCCATCAAGAAGGGTATCTTGCAAACCGTCAAGGTCGTCGACGAAT | |
| | TGGTCAAGGTCATGGGTAGACACAAGCCAGAAAACATCGTCATCGAAATGGCCAGAGAAAACCAAACCACCCAAAAGGGTCAAAAG | |
| | AACAGCAGAGAAAGAATGAAGAGAATCGAAGAAGGTATCAAGGAATTGGGTTCTCAAATCTTGAAGGAACACCCAGTCGAAAACAC | |
| | CCAATTGCAAAACGAAAAGTTGTACTTGTACTACTTGCAAAACGGTAGAGACATGTACGTCGACCAAGAATTGGACATCAACAGAT | |
| | TGTCTGACTACGACGTCGACCACATCGTCCCACAATCTTTCTTGAAGGACGACTCTATCGACAACAAGGTCTTGACCAGATCTGAC | |
| | AAGAACAGAGGTAAGTCTGACAACGTCCCATCTGAAGAAGTCGTCAAGAAGATGAAGAACTACTGGAGACAATTGTTGAACGCCAA | |
| | GTTGATCACCCAAAGAAAGTTCGACAACTTGACCAAGGCCGAAAGAGGTGGTTTGTCTGAATTGGACAAGGCCGGTTTCATCAAGA | |
| | GACAATTGGTCGAAACCAGACAAATCACCAAGCACGTCGCCCAAATCTTGGACAGCAGAATGAACACCAAGTACGACGAAAACGAC | |
| | AAGTTGATCAGAGAAGTCAAGGTCATCACCTTGAAGTCTAAGTTGGTCTCTGACTTCAGAAAGGACTTCCAATTCTACAAGGTCAG | |
| | AGAAATCAACAACTACCACCACGCCCACGACGCCTACTTGAACGCCGTCGTCGGTACCGCCTTGATCAAGAAGTACCCAAAGTTGG | |
| | AATCTGAATTCGTCTACGGTGACTACAAGGTCTACGACGTCAGAAAGATGATCGCCAAGTCTGAACAAGAAATCGGTAAGGCCACC | |
| | GCCAAGTACTTCTTCTACTCTAACATCATGAACTTCTTCAAGACCGAAATCACCTTGGCCAACGGTGAAATCAGAAAGAGACCATT | |
| | GATCGAAACCAACGGTGAAACCGGTGAAATCGTCTGGGACAAGGGTAGAGACTTCGCCACCGTCAGAAAGGTCTTGTCTATGCCAC | |
| | AAGTCAACATCGTCAAGAAGACCGAAGTCCAAACCGGTGGTTTCTCTAAGGAATCTATCTTGCCAAAGAGAAACTCTGACAAGTTG | |
| | ATCGCCAGAAAGAAGGACTGGGACCCAAAGAAGTACGGTGGTTTCGACTCTCCAACCGTCGCCTACTCTGTCTTGGTCGTCGCCAA | |
| | GGTCGAAAAGGGTAAGTCTAAGAAGTTGAAGTCTGTCAAGGAATTGTTGGGTATCACCATCATGGAAAGATCTTCTTTCGAAAAGA | |
| | ACCCAATCGACTTCTTGGAAGCCAAGGGTTACAAGGAAGTCAAGAAGGACTTGATCATCAAGTTGCCAAAGTACTCTTTGTTCGAA | |
| | TTGGAAAACGGTAGAAAGAGAATGTTGGCCTCTGCCGGTGAATTGCAAAAGGGTAACGAATTGGCCTTGCCATCTAAGTACGTCAA | |
| | CTTCTTGTACTTGGCCTCTCACTACGAAAAGTTGAAGGGTTCTCCAGAAGACAACGAACAAAAGCAATTGTTCGTCGAACAACACA | |
| | AGCACTACTTGGACGAAATCATCGAACAAATCTCTGAATTCTCTAAGAGAGTCATCTTGGCCGACGCCAACTTGGACAAGGTCTTG | |
| | TCTGCCTACAACAAGCACAGAGACAAGCCAATCAGAGAACAAGCCGAAAACATCATCCACTTGTTCACCTTGACCAACTTGGGTGC | |
| | CCCAGCCGCCTTCAAGTACTTCGACACCACCATCGACAGAAAGAGATACACCTCTACCAAGGAAGTCTTGGACGCCACCTTGATCC | |
| | ACCAATCTATCACCGGTTTGTACGAAACCAGAATCGACTTGTCTCAATTGGGTGGTGACGGTGGTGGTTCTCCAAAGAAGAAGAGA | |
| | AAGGTC | |
| Cas9 ORF | GATAAAAAATATTCTATTGGTTTAGATATTGGTACTAATTCTGTTGGTTGGGCTGTTATTACTGATGAATATAAAGTTCCTTCTAA | 343 |
| using U rich | AAAATTTAAAGTTTTAGGTAATACTGATCGTCATTCTATTAAAAAAAATTTAATTGGTGCTTTATTATTTGATTCTGGTGAAACTG | |
| codons of | CTGAAGCTACTCGTTTAAAACGTACTGCTCGTCGTCGTTATACTCGTCGTAAAAATCGTATTTGTTATTTACAAGAAATTTTTTCT | |
| Table 4 (no | AATGAAATGGCTAAAGTTGATGATTCTTTTTTTCATCGTTTAGAAGAATCTTTTTTAGTTGAAGAAGATAAAAAACATGAACGTCA | |
| start or | TCCTATTTTTGGTAATATTGTTGATGAAGTTGCTTATCATGAAAAATATCCTACTATTTATCATTTACGTAAAAAATTAGTTGATT | |
| stop codons; | CTACTGATAAAGCTGATTTACGTTTAATTTATTTAGCTTTAGCTCATATGATTAAATTTCGTGGTCATTTTTTAATTGAAGGTGAT | |
| suitable for | TTAAATCCTGATAATTCTGATGTTGATAAATTATTTATTCAATTAGTTCAAACTTATAATCAATTATTTGAAGAAAATCCTATTAA | |
| inclusion in | TGCTTCTGGTGTTGATGCTAAAGCTATTTTATCTGCTCGTTTATCTAAATCTCGTCGTTTAGAAAATTTAATTGCTCAATTACCTG | |
| fusion | GTGAAAAAAAAAATGGTTTATTTGGTAATTTAATTGCTTTATCTTTAGGTTTAACTCCTAATTTTAAATCTAATTTTGATTTAGCT | |
| protein | GAAGATGCTAAATTACAATTATCTAAAGATACTTATGATGATGATTTAGATAATTTATTAGCTCAAATTGGTGATCAATATGCTGA | |
| coding | TTTATTTTTAGCTGCTAAAAATTTATCTGATGCTATTTTATTATCTGATATTTTACGTGTTAATACTGAAATTACTAAAGCTCCTT | |
| sequence) | TATCTGCTTCTATGATTAAACGTTATGATGAACATCATCAAGATTTAACTTTATTAAAAGCTTTAGTTCGTCAACAATTACCTGAA | |
| | AAATATAAAGAAATTTTTTTTGATCAATCTAAAAATGGTTATGCTGGTTATATTGATGGTGGTGCTTCTCAAGAAGAATTTTATAA | |
| | ATTTATTAAACCTATTTTAGAAAAAATGGATGGTACTGAAGAATTATTAGTTAAATTAAATCGTGAAGATTTATTACGTAAACAAC | |
| | GTACTTTTGATAATGGTTCTATTCCTCATCAAATTCATTTAGGTGAATTACATGCTATTTTACGTCGTCAAGAAGATTTTTATCCT | |
| | TTTTTAAAAGATAATCGTGAAAAAATTGAAAAAATTTTAACTTTTCGTATTCCTTATTATGTTGGTCCTTTAGCTCGTGGTAATTC | |
| | TCGTTTTGCTTGGATGACTCGTAAATCTGAAGAAACTATTACTCCTTGGAATTTTGAAGAAGTTGTTGATAAAGGTGCTTCTGCTC | |
| | AATCTTTTATTGAACGTATGACTAATTTTGATAAAAATTTACCTAATGAAAAAGTTTTACCTAAACATTCTTTATTATATGAATAT | |
| | TTTACTGTTTATAATGAATTAACTAAAGTTAAATATGTTACTGAAGGTATGCGTAAACCTGCTTTTTTATCTGGTGAACAAAAAAA | |
| | AGCTATTGTTGATTTATTATTTAAAACTAATCGTAAAGTTACTGTTAAACAATTAAAAGAAGATTATTTTAAAAAAATTGAATGTT | |
| | TTGATTCTGTTGAAATTTCTGGTGTTGAAGATCGTTTTAATGCTTCTTTAGGTACTTATCATGATTTATTAAAAATTATTAAAGAT | |
| | AAAGATTTTTTAGATAATGAAGAAAATGAAGATATTTTAGAAGATATTGTTTTAACTTTAACTTTATTTGAAGATCGTGAAATGAT | |
| | TGAAGAACGTTTAAAAACTTATGCTCATTTATTTGATGATAAAGTTATGAAACAATTAAAACGTCGTCGTTATACTGGTTGGGGTC | |
| | GTTTATCTCGTAAATTAATTAATGGTATTCGTGATAAACAATCTGGTAAAACTATTTTAGATTTTTTAAAATCTGATGGTTTTGCT | |
| | AATCGTAATTTTATGCAATTAATTCATGATGATTCTTTAACTTTTAAAGAAGATATTCAAAAAGCTCAAGTTTCTGGTCAAGGTGA | |
| | TTCTTTACATGAACATATTGCTAATTTAGCTGGTTCTCCTGCTATTAAAAAAGGTATTTTACAAACTGTTAAAGTTGTTGATGAAT | |
| | TAGTTAAAGTTATGGGTCGTCATAAACCTGAAAATATTGTTATTGAAATGGCTCGTGAAAATCAAACTACTCAAAAAGGTCAAAAA | |
| | AATTCTCGTGAACGTATGAAACGTATTGAAGAAGGTATTAAAGAATTAGGTTCTCAAATTTTAAAAGAACATCCTGTTGAAAATAC | |
| | TCAATTACAAAATGAAAAATTATATTTATATTATTTACAAAATGGTCGTGATATGTATGTTGATCAAGAATTAGATATTAATCGTT | |
| | TATCTGATTATGATGTTGATCATATTGTTCCTCAATCTTTTTTAAAAGATGATTCTATTGATAATAAAGTTTTAACTCGTTCTGAT | |
| | AAAAATCGTGGTAAATCTGATAATGTTCCTTCTGAAGAAGTTGTTAAAAAAATGAAAAATTATTGGCGTCAATTATTAAATGCTAA | |
| | ATTAATTACTCAACGTAAATTTGATAATTTAACTAAAGCTGAACGTGGTGGTTTATCTGAATTAGATAAAGCTGGTTTTATTAAAC | |
| | GTCAATTAGTTGAAACTCGTCAAATTACTAAACATGTTGCTCAAATTTTAGATTCTCGTATGAATACTAAATATGATGAAAATGAT | |
| | AAATTAATTCGTGAAGTTAAAGTTATTACTTTAAAATCTAAATTAGTTTCTGATTTTCGTAAAGATTTTCAATTTTATAAAGTTCG | |
| | TGAAATTAATAATTATCATCATGCTCATGATGCTTATTTAAATGCTGTTGTTGGTACTGCTTTAATTAAAAAATATCCTAAATTAG | |
| | AATCTGAATTTGTTTATGGTGATTATAAAGTTTATGATGTTCGTAAAATGATTGCTAAATCTGAACAAGAAATTGGTAAAGCTACT | |
| | GCTAAATATTTTTTTTATTCTAATATTATGAATTTTTTTAAAACTGAAATTACTTTAGCTAATGGTGAAATTCGTAAACGTCCTTT | |
| | AATTGAAACTAATGGTGAAACTGGTGAAATTGTTTGGGATAAAGGTCGTGATTTTGCTACTGTTCGTAAAGTTTTATCTATGCCTC | |
| | AAGTTAATATTGTTAAAAAAACTGAAGTTCAAACTGGTGGTTTTTCTAAAGAATCTATTTTACCTAAACGTAATTCTGATAAATTA | |
| | ATTGCTCGTAAAAAAGATTGGGATCCTAAAAAATATGGTGGTTTTGATTCTCCTACTGTTGCTTATTCTGTTTTAGTTGTTGCTAA | |
| | AGTTGAAAAAGGTAAATCTAAAAAATTAAAATCTGTTAAAGAATTATTAGGTATTACTATTATGGAACGTTCTTCTTTTGAAAAAA | |
| | ATCCTATTGATTTTTTAGAAGCTAAAGGTTATAAAGAAGTTAAAAAAGATTTAATTATTAAATTACCTAAATATTCTTTATTTGAA | |
| | TTAGAAAATGGTCGTAAACGTATGTTAGCTTCTGCTGGTGAATTACAAAAAGGTAATGAATTAGCTTTACCTTCTAAATATGTTAA | |
| | TTTTTTATATTTAGCTTCTCATTATGAAAAATTAAAAGGTTCTCCTGAAGATAATGAACAAAAACAATTATTTGTTGAACAACATA | |
| | AACATTATTTAGATGAAATTATTGAACAAATTTCTGAATTTTCTAAACGTGTTATTTTAGCTGATGCTAATTTAGATAAAGTTTTA | |
| | TCTGCTTATAATAAACATCGTGATAAACCTATTCGTGAACAAGCTGAAAATATTATTCATTTATTTACTTTAACTAATTTAGGTGC | |
| | TCCTGCTGCTTTTAAATATTTTGATACTACTATTGATCGTAAACGTTATACTTCTACTAAAGAAGTTTTAGATGCTACTTTAATTC | |
| | ATCAATCTATTACTGGTTTATATGAAACTCGTATTGATTTATCTCAATTAGGTGGTGATGGTGGTGGTTCTCCTAAAAAAAAACGT | |
| | AAAGTT | |
| Cas9 ORF | GACAAAAAATACTCCATCGGCCTCGACATCGGCACCAACTCCGTCGGCTGGGCCGTCATCACCGACGAATACAAAGTCCCCTCCAA | 344 |
| using low G | AAAATTCAAAGTCCTCGGCAACACCGACAGACACTCCATCAAAAAAAACCTCATCGGCGCCCTCCTCTTCGACTCCGGCGAAACCG | |
| codons of | CCGAAGCCACCAGACTCAAAAGAACCGCCAGAAGAAGATACACCAGAAGAAAAAACAGAATCTGCTACCTCCAAGAAATCTTCTCC | |
| Table 4 (no | AACGAAATGGCCAAAGTCGACGACTCCTTCTTCCACAGACTCGAAGAATCCTTCCTCGTCGAAGAAGACAAAAAACACGAAAGACA | |
| start or | CCCCATCTTCGGCAACATCGTCGACGAAGTCGCCTACCACGAAAAATACCCCACCATCTACCACCTCAGAAAAAAACTCGTCGACT | |
| stop codons; | CCACCGACAAAGCCGACCTCAGACTCATCTACCTCGCCCTCGCCCACATGATCAAATTCAGAGGCCACTTCCTCATCGAAGGCGAC | |
| suitable for | CTCAACCCCGACAACTCCGACGTCGACAAACTCTTCATCCAACTCGTCCAAACCTACAACCAACTCTTCGAAGAAAACCCCATCAA | |
| inclusion in | CGCCTCCGGCGTCGACGCCAAAGCCATCCTCTCCGCCAGACTCTCCAAATCCAGAAGACTCGAAAACCTCATCGCCCAACTCCCCG | |
| fusion | GCGAAAAAAAAAACGGCCTCTTCGGCAACCTCATCGCCCTCTCCCTCGGCCTCACCCCCAACTTCAAATCCAACTTCGACCTCGCC | |
| protein | GAAGACGCCAAACTCCAACTCTCCAAAGACACCTACGACGACGACCTCGACAACCTCCTCGCCCAAATCGGCGACCAATACGCCGA | |
| coding | CCTCTTCCTCGCCGCCAAAAACCTCTCCGACGCCATCCTCCTCTCCGACATCCTCAGAGTCAACACCGAAATCACCAAAGCCCCCC | |
| sequence) | TCTCCGCCTCCATGATCAAAAGATACGACGAACACCACCAAGACCTCACCCTCCTCAAAGCCCTCGTCAGACAACAACTCCCCGAA | |
| | AAATACAAAGAAATCTTCTTCGACCAATCCAAAAACGGCTACGCCGGCTACATCGACGGCGGCGCCTCCCAAGAAGAATTCTACAA | |
| | ATTCATCAAACCCATCCTCGAAAAAATGGACGGCACCGAAGAACTCCTCGTCAAACTCAACAGAGAAGACCTCCTCAGAAAACAAA | |
| | GAACCTTCGACAACGGCTCCATCCCCCACCAAATCCACCTCGGCGAACTCCACGCCATCCTCAGAAGACAAGAAGACTTCTACCCC | |
| | TTCCTCAAAGACAACAGAGAAAAAATCGAAAAAATCCTCACCTTCAGAATCCCCTACTACGTCGGCCCCCTCGCCAGAGGCAACTC | |
| | CAGATTCGCCTGGATGACCAGAAAATCCGAAGAAACCATCACCCCCTGGAACTTCGAAGAAGTCGTCGACAAAGGCGCCTCCGCCC | |
| | AATCCTTCATCGAAAGAATGACCAACTTCGACAAAAACCTCCCCAACGAAAAAGTCCTCCCCAAACACTCCCTCCTCTACGAATAC | |
| | TTCACCGTCTACAACGAACTCACCAAAGTCAAATACGTCACCGAAGGCATGAGAAAACCCGCCTTCCTCTCCGGCGAACAAAAAAA | |
| | AGCCATCGTCGACCTCCTCTTCAAAACCAACAGAAAAGTCACCGTCAAACAACTCAAAGAAGACTACTTCAAAAAAATCGAATGCT | |
| | TCGACTCCGTCGAAATCTCCGGCGTCGAAGACAGATTCAACGCCTCCCTCGGCACCTACCACGACCTCCTCAAAATCATCAAAGAC | |
| | AAAGACTTCCTCGACAACGAAGAAAACGAAGACATCCTCGAAGACATCGTCCTCACCCTCACCCTCTTCGAAGACAGAGAAATGAT | |
| | CGAAGAAAGACTCAAAACCTACGCCCACCTCTTCGACGACAAAGTCATGAAACAACTCAAAAGAAGAAGATACACCGGCTGGGGCA | |
| | GACTCTCCAGAAAACTCATCAACGGCATCAGAGACAAACAATCCGGCAAAACCATCCTCGACTTCCTCAAATCCGACGGCTTCGCC | |
| | AACAGAAACTTCATGCAACTCATCCACGACGACTCCCTCACCTTCAAAGAAGACATCCAAAAAGCCCAAGTCTCCGGCCAAGGCGA | |
| | CTCCCTCCACGAACACATCGCCAACCTCGCCGGCTCCCCCGCCATCAAAAAAGGCATCCTCCAAACCGTCAAAGTCGTCGACGAAC | |
| | TCGTCAAAGTCATGGGCAGACACAAACCCGAAAACATCGTCATCGAAATGGCCAGAGAAAACCAAACCACCCAAAAAGGCCAAAAA | |
| | AACTCCAGAGAAAGAATGAAAAGAATCGAAGAAGGCATCAAAGAACTCGGCTCCCAAATCCTCAAAGAACACCCCGTCGAAAACAC | |
| | CCAACTCCAAAACGAAAAACTCTACCTCTACTACCTCCAAAACGGCAGAGACATGTACGTCGACCAAGAACTCGACATCAACAGAC | |
| | TCTCCGACTACGACGTCGACCACATCGTCCCCCAATCCTTCCTCAAAGACGACTCCATCGACAACAAAGTCCTCACCAGATCCGAC | |
| | AAAAACAGAGGCAAATCCGACAACGTCCCCTCCGAAGAAGTCGTCAAAAAAATGAAAAACTACTGGAGACAACTCCTCAACGCCAA | |
| | ACTCATCACCCAAAGAAAATTCGACAACCTCACCAAAGCCGAAAGAGGCGGCCTCTCCGAACTCGACAAAGCCGGCTTCATCAAAA | |
| | GACAACTCGTCGAAACCAGACAAATCACCAAACACGTCGCCCAAATCCTCGACTCCAGAATGAACACCAAATACGACGAAAACGAC | |
| | AAACTCATCAGAGAAGTCAAAGTCATCACCCTCAAATCCAAACTCGTCTCCGACTTCAGAAAAGACTTCCAATTCTACAAAGTCAG | |
| | AGAAATCAACAACTACCACCACGCCCACGACGCCTACCTCAACGCCGTCGTCGGCACCGCCCTCATCAAAAAATACCCCAAACTCG | |
| | AATCCGAATTCGTCTACGGCGACTACAAAGTCTACGACGTCAGAAAAATGATCGCCAAATCCGAACAAGAAATCGGCAAAGCCACC | |
| | GCCAAATACTTCTTCTACTCCAACATCATGAACTTCTTCAAAACCGAAATCACCCTCGCCAACGGCGAAATCAGAAAAAGACCCCT | |
| | CATCGAAACCAACGGCGAAACCGGCGAAATCGTCTGGGACAAAGGCAGAGACTTCGCCACCGTCAGAAAAGTCCTCTCCATGCCCC | |
| | AAGTCAACATCGTCAAAAAAACCGAAGTCCAAACCGGCGGCTTCTCCAAAGAATCCATCCTCCCCAAAAGAAACTCCGACAAACTC | |
| | ATCGCCAGAAAAAAAGACTGGGACCCCAAAAAATACGGCGGCTTCGACTCCCCCACCGTCGCCTACTCCGTCCTCGTCGTCGCCAA | |
| | AGTCGAAAAAGGCAAATCCAAAAAACTCAAATCCGTCAAAGAACTCCTCGGCATCACCATCATGGAAAGATCCTCCTTCGAAAAAA | |
| | ACCCCATCGACTTCCTCGAAGCCAAAGGCTACAAAGAAGTCAAAAAAGACCTCATCATCAAACTCCCCAAATACTCCCTCTTCGAA | |
| | CTCGAAAACGGCAGAAAAAGAATGCTCGCCTCCGCCGGCGAACTCCAAAAAGGCAACGAACTCGCCCTCCCCTCCAAATACGTCAA | |
| | CTTCCTCTACCTCGCCTCCCACTACGAAAAACTCAAAGGCTCCCCCGAAGACAACGAACAAAAACAACTCTTCGTCGAACAACACA | |
| | AACACTACCTCGACGAAATCATCGAACAAATCTCCGAATTCTCCAAAAGAGTCATCCTCGCCGACGCCAACCTCGACAAAGTCCTC | |
| | TCCGCCTACAACAAACACAGAGACAAACCCATCAGAGAACAAGCCGAAAACATCATCCACCTCTTCACCCTCACCAACCTCGGCGC | |
| | CCCCGCCGCCTTCAAATACTTCGACACCACCATCGACAGAAAAAGATACACCTCCACCAAAGAAGTCCTCGACGCCACCCTCATCC | |
| | ACCAATCCATCACCGGCCTCTACGAAACCAGAATCGACCTCTCCCAACTCGGCGGCGACGGCGGCGGCTCCCCCAAAAAAAAAAGA | |
| | AAAGTC | |
| Cas9 ORF | GATAAGAAGTATAGTATTGGATTGGATATTGGAACAAATAGTGTGGGATGGGCTGTGATTACAGATGAGTATAAGGTGCCTAGTAA | 345 |
| using low C | GAAGTTTAAGGTGTTGGGAAATACAGATAGACATAGTATTAAGAAGAATTTGATTGGAGCTTTGTTGTTTGATAGTGGAGAGACAG | |
| codons of | CTGAGGCTACAAGATTGAAGAGAACAGCTAGAAGAAGATATACAAGAAGAAAGAATAGAATTTGTTATTTGCAGGAGATTTTTAGT | |
| Table 4 (no | AATGAGATGGCTAAGGTGGATGATAGTTTTTTTCATAGATTGGAGGAGAGTTTTTTGGTGGAGGAGGATAAGAAGCATGAGAGACA | |
| start or | TCCTATTTTTGGAAATATTGTGGATGAGGTGGCTTATCATGAGAAGTATCCTACAATTTATCATTTGAGAAAGAAGTTGGTGGATA | |
| stop codons; | GTACAGATAAGGCTGATTTGAGATTGATTTATTTGGCTTTGGCTCATATGATTAAGTTTAGAGGACATTTTTTGATTGAGGGAGAT | |
| suitable for | TTGAATCCTGATAATAGTGATGTGGATAAGTTGTTTATTCAGTTGGTGCAGACATATAATCAGTTGTTTGAGGAGAATCCTATTAA | |
| inclusion in | TGCTAGTGGAGTGGATGCTAAGGCTATTTTGAGTGCTAGATTGAGTAAGAGTAGAAGATTGGAGAATTTGATTGCTCAGTTGCCTG | |
| fusion | GAGAGAAGAAGAATGGATTGTTTGGAAATTTGATTGCTTTGAGTTTGGGATTGACACCTAATTTTAAGAGTAATTTTGATTTGGCT | |
| protein | GAGGATGCTAAGTTGCAGTTGAGTAAGGATACATATGATGATGATTTGGATAATTTGTTGGCTCAGATTGGAGATCAGTATGCTGA | |
| coding | TTTGTTTTTGGCTGCTAAGAATTTGAGTGATGCTATTTTGTTGAGTGATATTTTGAGAGTGAATACAGAGATTACAAAGGCTCCTT | |
| sequence) | TGAGTGCTAGTATGATTAAGAGATATGATGAGCATCATCAGGATTTGACATTGTTGAAGGCTTTGGTGAGACAGCAGTTGCCTGAG | |
| | AAGTATAAGGAGATTTTTTTTGATCAGAGTAAGAATGGATATGCTGGATATATTGATGGAGGAGCTAGTCAGGAGGAGTTTTATAA | |
| | GTTTATTAAGCCTATTTTGGAGAAGATGGATGGAACAGAGGAGTTGTTGGTGAAGTTGAATAGAGAGGATTTGTTGAGAAAGCAGA | |
| | GAACATTTGATAATGGAAGTATTCCTCATCAGATTCATTTGGGAGAGTTGCATGCTATTTTGAGAAGACAGGAGGATTTTTATCCT | |
| | TTTTTGAAGGATAATAGAGAGAAGATTGAGAAGATTTTGACATTTAGAATTCCTTATTATGTGGGACCTTTGGCTAGAGGAAATAG | |
| | TAGATTTGCTTGGATGACAAGAAAGAGTGAGGAGACAATTACACCTTGGAATTTTGAGGAGGTGGTGGATAAGGGAGCTAGTGCTC | |
| | AGAGTTTTATTGAGAGAATGACAAATTTTGATAAGAATTTGCCTAATGAGAAGGTGTTGCCTAAGCATAGTTTGTTGTATGAGTAT | |
| | TTTACAGTGTATAATGAGTTGACAAAGGTGAAGTATGTGACAGAGGGAATGAGAAAGCCTGCTTTTTTGAGTGGAGAGCAGAAGAA | |
| | GGCTATTGTGGATTTGTTGTTTAAGACAAATAGAAAGGTGACAGTGAAGCAGTTGAAGGAGGATTATTTTAAGAAGATTGAGTGTT | |
| | TTGATAGTGTGGAGATTAGTGGAGTGGAGGATAGATTTAATGCTAGTTTGGGAACATATCATGATTTGTTGAAGATTATTAAGGAT | |
| | AAGGATTTTTTGGATAATGAGGAGAATGAGGATATTTTGGAGGATATTGTGTTGACATTGACATTGTTTGAGGATAGAGAGATGAT | |
| | TGAGGAGAGATTGAAGACATATGCTCATTTGTTTGATGATAAGGTGATGAAGCAGTTGAAGAGAAGAAGATATACAGGATGGGGAA | |
| | GATTGAGTAGAAAGTTGATTAATGGAATTAGAGATAAGCAGAGTGGAAAGACAATTTTGGATTTTTTGAAGAGTGATGGATTTGCT | |
| | AATAGAAATTTTATGCAGTTGATTCATGATGATAGTTTGACATTTAAGGAGGATATTCAGAAGGCTCAGGTGAGTGGACAGGGAGA | |
| | TAGTTTGCATGAGCATATTGCTAATTTGGCTGGAAGTCCTGCTATTAAGAAGGGAATTTTGCAGACAGTGAAGGTGGTGGATGAGT | |
| | TGGTGAAGGTGATGGGAAGACATAAGCCTGAGAATATTGTGATTGAGATGGCTAGAGAGAATCAGACAACACAGAAGGGACAGAAG | |
| | AATAGTAGAGAGAGAATGAAGAGAATTGAGGAGGGAATTAAGGAGTTGGGAAGTCAGATTTTGAAGGAGCATCCTGTGGAGAATAC | |
| | ACAGTTGCAGAATGAGAAGTTGTATTTGTATTATTTGCAGAATGGAAGAGATATGTATGTGGATCAGGAGTTGGATATTAATAGAT | |
| | TGAGTGATTATGATGTGGATCATATTGTGCCTCAGAGTTTTTTGAAGGATGATAGTATTGATAATAAGGTGTTGACAAGAAGTGAT | |
| | AAGAATAGAGGAAAGAGTGATAATGTGCCTAGTGAGGAGGTGGTGAAGAAGATGAAGAATTATTGGAGACAGTTGTTGAATGCTAA | |
| | GTTGATTACACAGAGAAAGTTTGATAATTTGACAAAGGCTGAGAGAGGAGGATTGAGTGAGTTGGATAAGGCTGGATTTATTAAGA | |
| | GACAGTTGGTGGAGACAAGACAGATTACAAAGCATGTGGCTCAGATTTTGGATAGTAGAATGAATACAAAGTATGATGAGAATGAT | |
| | AAGTTGATTAGAGAGGTGAAGGTGATTACATTGAAGAGTAAGTTGGTGAGTGATTTTAGAAAGGATTTTCAGTTTTATAAGGTGAG | |
| | AGAGATTAATAATTATCATCATGCTCATGATGCTTATTTGAATGCTGTGGTGGGAACAGCTTTGATTAAGAAGTATCCTAAGTTGG | |
| | AGAGTGAGTTTGTGTATGGAGATTATAAGGTGTATGATGTGAGAAAGATGATTGCTAAGAGTGAGCAGGAGATTGGAAAGGCTACA | |
| | GCTAAGTATTTTTTTTATAGTAATATTATGAATTTTTTTAAGACAGAGATTACATTGGCTAATGGAGAGATTAGAAAGAGACCTTT | |
| | GATTGAGACAAATGGAGAGACAGGAGAGATTGTGTGGGATAAGGGAAGAGATTTTGCTACAGTGAGAAAGGTGTTGAGTATGCCTC | |
| | AGGTGAATATTGTGAAGAAGACAGAGGTGCAGACAGGAGGATTTAGTAAGGAGAGTATTTTGCCTAAGAGAAATAGTGATAAGTTG | |
| | ATTGCTAGAAAGAAGGATTGGGATCCTAAGAAGTATGGAGGATTTGATAGTCCTACAGTGGCTTATAGTGTGTTGGTGGTGGCTAA | |
| | GGTGGAGAAGGGAAAGAGTAAGAAGTTGAAGAGTGTGAAGGAGTTGTTGGGAATTACAATTATGGAGAGAAGTAGTTTTGAGAAGA | |
| | ATCCTATTGATTTTTTGGAGGCTAAGGGATATAAGGAGGTGAAGAAGGATTTGATTATTAAGTTGCCTAAGTATAGTTTGTTTGAG | |
| | TTGGAGAATGGAAGAAAGAGAATGTTGGCTAGTGCTGGAGAGTTGCAGAAGGGAAATGAGTTGGCTTTGCCTAGTAAGTATGTGAA | |
| | TTTTTTGTATTTGGCTAGTCATTATGAGAAGTTGAAGGGAAGTCCTGAGGATAATGAGCAGAAGCAGTTGTTTGTGGAGCAGCATA | |
| | AGCATTATTTGGATGAGATTATTGAGCAGATTAGTGAGTTTAGTAAGAGAGTGATTTTGGCTGATGCTAATTTGGATAAGGTGTTG | |
| | AGTGCTTATAATAAGCATAGAGATAAGCCTATTAGAGAGCAGGCTGAGAATATTATTCATTTGTTTACATTGACAAATTTGGGAGC | |
| | TCCTGCTGCTTTTAAGTATTTTGATACAACAATTGATAGAAAGAGATATACAAGTACAAAGGAGGTGTTGGATGCTACATTGATTC | |
| | ATCAGAGTATTACAGGATTGTATGAGACAAGAATTGATTTGAGTCAGTTGGGAGGAGATGGAGGAGGAAGTCCTAAGAAGAAGAGA | |
| | AAGGTG | |
| Cas9 ORF | GACAAGAAGTACTCCATCGGCCTGGACATCGGCACCAACTCCGTGGGCTGGGCCGTGATCACCGACGAGTACAAGGTGCCCTCCAA | 346 |
| using low A | GAAGTTCAAGGTGCTGGGCAACACCGACCGGCACTCCATCAAGAAGAACCTGATCGGCGCCCTGCTGTTCGACTCCGGCGAGACCG | |
| codons of | CCGAGGCCACCCGGCTGAAGCGGACCGCCCGGCGGCGGTACACCCGGCGGAAGAACCGGATCTGCTACCTGCAGGAGATCTTCTCC | |
| Table 4 (no | AACGAGATGGCCAAGGTGGACGACTCCTTCTTCCACCGGCTGGAGGAGTCCTTCCTGGTGGAGGAGGACAAGAAGCACGAGCGGCA | |
| start or | CCCCATCTTCGGCAACATCGTGGACGAGGTGGCCTACCACGAGAAGTACCCCACCATCTACCACCTGCGGAAGAAGCTGGTGGACT | |
| stop codons; | CCACCGACAAGGCCGACCTGCGGCTGATCTACCTGGCCCTGGCCCACATGATCAAGTTCCGGGGCCACTTCCTGATCGAGGGCGAC | |
| suitable for | CTGAACCCCGACAACTCCGACGTGGACAAGCTGTTCATCCAGCTGGTGCAGACCTACAACCAGCTGTTCGAGGAGAACCCCATCAA | |
| inclusion in | CGCCTCCGGCGTGGACGCCAAGGCCATCCTGTCCGCCCGGCTGTCCAAGTCCCGGCGGCTGGAGAACCTGATCGCCCAGCTGCCCG | |
| fusion | GCGAGAAGAAGAACGGCCTGTTCGGCAACCTGATCGCCCTGTCCCTGGGCCTGACCCCCAACTTCAAGTCCAACTTCGACCTGGCC | |
| protein | GAGGACGCCAAGCTGCAGCTGTCCAAGGACACCTACGACGACGACCTGGACAACCTGCTGGCCCAGATCGGCGACCAGTACGCCGA | |
| coding | CCTGTTCCTGGCCGCCAAGAACCTGTCCGACGCCATCCTGCTGTCCGACATCCTGCGGGTGAACACCGAGATCACCAAGGCCCCCC | |
| sequence) | TGTCCGCCTCCATGATCAAGCGGTACGACGAGCACCACCAGGACCTGACCCTGCTGAAGGCCCTGGTGCGGCAGCAGCTGCCCGAG | |
| | AAGTACAAGGAGATCTTCTTCGACCAGTCCAAGAACGGCTACGCCGGCTACATCGACGGCGGCGCCTCCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACCACATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCGGCGGCTCCCCCAAGAAGAAGCGG | |
| | AAGGTG | |
| Cas9 ORF | GACAAGAAGTACAGCATCGGCCTGGACATCGGCACCAACAGCGTGGGCTGGGCCGTGATCACCGACGAGTACAAGGTGCCCAGCAA | 347 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4 | AACGAGATGGCCAAGGTGGACGACAGCTTCTTCCACCGGCTGGAGGAGAGCTTCCTGGTGGAGGAGGACAAGAAGCACGAGCGGCA | |
| (no start or | CCCCATCTTCGGCAACATCGTGGACGAGGTGGCCTACCACGAGAAGTACCCCACCATCTACCACCTGCGGAAGAAGCTGGTGGACA | |
| stop codons; | GCACCGACAAGGCCGACCTGCGGCTGATCTACCTGGCCCTGGCCCACATGATCAAGTTCCGGGGCCACTTCCTGATCGAGGGCGAC | |
| suitable for | CTGAACCCCGACAACAGCGACGTGGACAAGCTGTTCATCCAGCTGGTGCAGACCTACAACCAGCTGTTCGAGGAGAACCCCATCAA | |
| inclusion in | CGCCAGCGGCGTGGACGCCAAGGCCATCCTGAGCGCCCGGCTGAGCAAGAGCCGGCGGCTGGAGAACCTGATCGCCCAGCTGCCCG | |
| fusion | GCGAGAAGAAGAACGGCCTGTTCGGCAACCTGATCGCCCTGAGCCTGGGCCTGACCCCCAACTTCAAGAGCAACTTCGACCTGGCC | |
| protein | GAGGACGCCAAGCTGCAGCTGAGCAAGGACACCTACGACGACGACCTGGACAACCTGCTGGCCCAGATCGGCGACCAGTACGCCGA | |
| coding | CCTGTTCCTGGCCGCCAAGAACCTGAGCGACGCCATCCTGCTGAGCGACATCCTGCGGGTGAACACCGAGATCACCAAGGCCCCCC | |
| sequence) | TGAGCGCCAGCATGATCAAGCGGTACGACGAGCACCACCAGGACCTGACCCTGCTGAAGGCCCTGGTGCGGCAGCAGCTGCCCGAG | |
| | AAGTACAAGGAGATCTTCTTCGACCAGAGCAAGAACGGCTACGCCGGCTACATCGACGGCGGCGCCAGCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCGGCGGCAGCCCCAAGAAGAAGCGG | |
| | AAGGTG | |
| Cas9 ORF | GACAAGAAGTACTCCATCGGCCTGGACATCGGCACCAACTCCGTGGGCTGGGCCGTGATCACCGACGAGTACAAGGTGCCCTCCAA | 348 |
| using low A | GAAGTTCAAGGTGCTGGGCAACACCGACCGGCACTCCATCAAGAAGAACCTGATCGGCGCCCTGCTGTTCGACTCCGGCGAGACCG | |
| codons of | CCGAGGCCACCCGGCTGAAGCGGACCGCCCGGCGGCGGTACACCCGGCGGAAGAACCGGATCTGCTACCTGCAGGAGATCTTCTCC | |
| Table 4, | AACGAGATGGCCAAGGTGGACGACTCCTTCTTCCACCGGCTGGAGGAGTCCTTCCTGGTGGAGGAGGACAAGAAGCACGAGCGGCA | |
| with two C- | CCCCATCTTCGGCAACATCGTGGACGAGGTGGCCTACCACGAGAAGTACCCCACCATCTACCACCTGCGGAAGAAGCTGGTGGACT | |
| terminal NLS | CCACCGACAAGGCCGACCTGCGGCTGATCTACCTGGCCCTGGCCCACATGATCAAGTTCCGGGGCCACTTCCTGATCGAGGGCGAC | |
| sequences | CTGAACCCCGACAACTCCGACGTGGACAAGCTGTTCATCCAGCTGGTGCAGACCTACAACCAGCTGTTCGAGGAGAACCCCATCAA | |
| (no start or | CGCCTCCGGCGTGGACGCCAAGGCCATCCTGTCCGCCCGGCTGTCCAAGTCCCGGCGGCTGGAGAACCTGATCGCCCAGCTGCCCG | |
| stop codons; | GCGAGAAGAAGAACGGCCTGTTCGGCAACCTGATCGCCCTGTCCCTGGGCCTGACCCCCAACTTCAAGTCCAACTTCGACCTGGCC | |
| suitable for | GAGGACGCCAAGCTGCAGCTGTCCAAGGACACCTACGACGACGACCTGGACAACCTGCTGGCCCAGATCGGCGACCAGTACGCCGA | |
| inclusion in | CCTGTTCCTGGCCGCCAAGAACCTGTCCGACGCCATCCTGCTGTCCGACATCCTGCGGGTGAACACCGAGATCACCAAGGCCCCCC | |
| fusion | TGTCCGCCTCCATGATCAAGCGGTACGACGAGCACCACCAGGACCTGACCCTGCTGAAGGCCCTGGTGCGGCAGCAGCTGCCCGAG | |
| protein | AAGTACAAGGAGATCTTCTTCGACCAGTCCAAGAACGGCTACGCCGGCTACATCGACGGCGGCGCCTCCCAGGAGGAGTTCTACAA | |
| coding | GTTCATCAAGCCCATCCTGGAGAAGATGGACGGCACCGAGGAGCTGCTGGTGAAGCTGAACCGGGAGGACCTGCTGCGGAAGCAGC | |
| sequence) | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACCACATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCTCCGGCTCCCCCAAGAAGAAGCGG | |
| | AAGGTGGACGGCTCCCCCAAGAAGAAGCGGAAGGTGGACTCCGGC | |
| Cas9 nickase | GACAAGAAGTACTCCATCGGCCTGGCCATCGGCACCAACTCCGTGGGCTGGGCCGTGATCACCGACGAGTACAAGGTGCCCTCCAA | 349 |
| ORF using | GAAGTTCAAGGTGCTGGGCAACACCGACCGGCACTCCATCAAGAAGAACCTGATCGGCGCCCTGCTGTTCGACTCCGGCGAGACCG | |
| low A codons | CCGAGGCCACCCGGCTGAAGCGGACCGCCCGGCGGCGGTACACCCGGCGGAAGAACCGGATCTGCTACCTGCAGGAGATCTTCTCC | |
| of Table 4 | AACGAGATGGCCAAGGTGGACGACTCCTTCTTCCACCGGCTGGAGGAGTCCTTCCTGGTGGAGGAGGACAAGAAGCACGAGCGGCA | |
| (no start or | CCCCATCTTCGGCAACATCGTGGACGAGGTGGCCTACCACGAGAAGTACCCCACCATCTACCACCTGCGGAAGAAGCTGGTGGACT | |
| stop codons; | CCACCGACAAGGCCGACCTGCGGCTGATCTACCTGGCCCTGGCCCACATGATCAAGTTCCGGGGCCACTTCCTGATCGAGGGCGAC | |
| suitable for | CTGAACCCCGACAACTCCGACGTGGACAAGCTGTTCATCCAGCTGGTGCAGACCTACAACCAGCTGTTCGAGGAGAACCCCATCAA | |
| inclusion in | CGCCTCCGGCGTGGACGCCAAGGCCATCCTGTCCGCCCGGCTGTCCAAGTCCCGGCGGCTGGAGAACCTGATCGCCCAGCTGCCCG | |
| fusion | GCGAGAAGAAGAACGGCCTGTTCGGCAACCTGATCGCCCTGTCCCTGGGCCTGACCCCCAACTTCAAGTCCAACTTCGACCTGGCC | |
| protein | GAGGACGCCAAGCTGCAGCTGTCCAAGGACACCTACGACGACGACCTGGACAACCTGCTGGCCCAGATCGGCGACCAGTACGCCGA | |
| coding | CCTGTTCCTGGCCGCCAAGAACCTGTCCGACGCCATCCTGCTGTCCGACATCCTGCGGGTGAACACCGAGATCACCAAGGCCCCCC | |
| sequence) | TGTCCGCCTCCATGATCAAGCGGTACGACGAGCACCACCAGGACCTGACCCTGCTGAAGGCCCTGGTGCGGCAGCAGCTGCCCGAG | |
| | AAGTACAAGGAGATCTTCTTCGACCAGTCCAAGAACGGCTACGCCGGCTACATCGACGGCGGCGCCTCCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACCACATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCGGCGGCTCCCCCAAGAAGAAGCGG | |
| | AAGGTG | |
| Cas9 nickase | GACAAGAAGTACTCCATCGGCCTGGCCATCGGCACCAACTCCGTGGGCTGGGCCGTGATCACCGACGAGTACAAGGTGCCCTCCAA | 350 |
| ORF using | GAAGTTCAAGGTGCTGGGCAACACCGACCGGCACTCCATCAAGAAGAACCTGATCGGCGCCCTGCTGTTCGACTCCGGCGAGACCG | |
| low A codons | CCGAGGCCACCCGGCTGAAGCGGACCGCCCGGCGGCGGTACACCCGGCGGAAGAACCGGATCTGCTACCTGCAGGAGATCTTCTCC | |
| of Table 4 | AACGAGATGGCCAAGGTGGACGACTCCTTCTTCCACCGGCTGGAGGAGTCCTTCCTGGTGGAGGAGGACAAGAAGCACGAGCGGCA | |
| (no NLS and | CCCCATCTTCGGCAACATCGTGGACGAGGTGGCCTACCACGAGAAGTACCCCACCATCTACCACCTGCGGAAGAAGCTGGTGGACT | |
| no start or | CCACCGACAAGGCCGACCTGCGGCTGATCTACCTGGCCCTGGCCCACATGATCAAGTTCCGGGGCCACTTCCTGATCGAGGGCGAC | |
| stop codons; | CTGAACCCCGACAACTCCGACGTGGACAAGCTGTTCATCCAGCTGGTGCAGACCTACAACCAGCTGTTCGAGGAGAACCCCATCAA | |
| suitable for | CGCCTCCGGCGTGGACGCCAAGGCCATCCTGTCCGCCCGGCTGTCCAAGTCCCGGCGGCTGGAGAACCTGATCGCCCAGCTGCCCG | |
| inclusion in | GCGAGAAGAAGAACGGCCTGTTCGGCAACCTGATCGCCCTGTCCCTGGGCCTGACCCCCAACTTCAAGTCCAACTTCGACCTGGCC | |
| fusion | GAGGACGCCAAGCTGCAGCTGTCCAAGGACACCTACGACGACGACCTGGACAACCTGCTGGCCCAGATCGGCGACCAGTACGCCGA | |
| protein | CCTGTTCCTGGCCGCCAAGAACCTGTCCGACGCCATCCTGCTGTCCGACATCCTGCGGGTGAACACCGAGATCACCAAGGCCCCCC | |
| coding | TGTCCGCCTCCATGATCAAGCGGTACGACGAGCACCACCAGGACCTGACCCTGCTGAAGGCCCTGGTGCGGCAGCAGCTGCCCGAG | |
| sequence) | AAGTACAAGGAGATCTTCTTCGACCAGTCCAAGAACGGCTACGCCGGCTACATCGACGGCGGCGCCTCCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACCACATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGAC | |
| Cas9 nickase | GACAAGAAGTACTCCATCGGCCTGGCCATCGGCACCAACTCCGTGGGCTGGGCCGTGATCACCGACGAGTACAAGGTGCCCTCCAA | 351 |
| ORF using | GAAGTTCAAGGTGCTGGGCAACACCGACCGGCACTCCATCAAGAAGAACCTGATCGGCGCCCTGCTGTTCGACTCCGGCGAGACCG | |
| low A codons | CCGAGGCCACCCGGCTGAAGCGGACCGCCCGGCGGCGGTACACCCGGCGGAAGAACCGGATCTGCTACCTGCAGGAGATCTTCTCC | |
| of Table 4, | AACGAGATGGCCAAGGTGGACGACTCCTTCTTCCACCGGCTGGAGGAGTCCTTCCTGGTGGAGGAGGACAAGAAGCACGAGCGGCA | |
| with two C- | CCCCATCTTCGGCAACATCGTGGACGAGGTGGCCTACCACGAGAAGTACCCCACCATCTACCACCTGCGGAAGAAGCTGGTGGACT | |
| terminal NLS | CCACCGACAAGGCCGACCTGCGGCTGATCTACCTGGCCCTGGCCCACATGATCAAGTTCCGGGGCCACTTCCTGATCGAGGGCGAC | |
| sequences | CTGAACCCCGACAACTCCGACGTGGACAAGCTGTTCATCCAGCTGGTGCAGACCTACAACCAGCTGTTCGAGGAGAACCCCATCAA | |
| (no start or | CGCCTCCGGCGTGGACGCCAAGGCCATCCTGTCCGCCCGGCTGTCCAAGTCCCGGCGGCTGGAGAACCTGATCGCCCAGCTGCCCG | |
| stop codons; | GCGAGAAGAAGAACGGCCTGTTCGGCAACCTGATCGCCCTGTCCCTGGGCCTGACCCCCAACTTCAAGTCCAACTTCGACCTGGCC | |
| suitable for | GAGGACGCCAAGCTGCAGCTGTCCAAGGACACCTACGACGACGACCTGGACAACCTGCTGGCCCAGATCGGCGACCAGTACGCCGA | |
| inclusion in | CCTGTTCCTGGCCGCCAAGAACCTGTCCGACGCCATCCTGCTGTCCGACATCCTGCGGGTGAACACCGAGATCACCAAGGCCCCCC | |
| fusion | TGTCCGCCTCCATGATCAAGCGGTACGACGAGCACCACCAGGACCTGACCCTGCTGAAGGCCCTGGTGCGGCAGCAGCTGCCCGAG | |
| protein | AAGTACAAGGAGATCTTCTTCGACCAGTCCAAGAACGGCTACGCCGGCTACATCGACGGCGGCGCCTCCCAGGAGGAGTTCTACAA | |
| coding | GTTCATCAAGCCCATCCTGGAGAAGATGGACGGCACCGAGGAGCTGCTGGTGAAGCTGAACCGGGAGGACCTGCTGCGGAAGCAGC | |
| sequence) | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACCACATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCTCCGGCTCCCCCAAGAAGAAGCGG | |
| | AAGGTGGACGGCTCCCCCAAGAAGAAGCGGAAGGTGGACTCCGGC | |
| dCas9 ORF | GACAAGAAGTACTCCATCGGCCTGGCCATCGGCACCAACTCCGTGGGCTGGGCCGTGATCACCGACGAGTACAAGGTGCCCTCCAA | 352 |
| using low A | GAAGTTCAAGGTGCTGGGCAACACCGACCGGCACTCCATCAAGAAGAACCTGATCGGCGCCCTGCTGTTCGACTCCGGCGAGACCG | |
| codons of | CCGAGGCCACCCGGCTGAAGCGGACCGCCCGGCGGCGGTACACCCGGCGGAAGAACCGGATCTGCTACCTGCAGGAGATCTTCTCC | |
| Table 4 (no | AACGAGATGGCCAAGGTGGACGACTCCTTCTTCCACCGGCTGGAGGAGTCCTTCCTGGTGGAGGAGGACAAGAAGCACGAGCGGCA | |
| start or | CCCCATCTTCGGCAACATCGTGGACGAGGTGGCCTACCACGAGAAGTACCCCACCATCTACCACCTGCGGAAGAAGCTGGTGGACT | |
| stop codons; | CCACCGACAAGGCCGACCTGCGGCTGATCTACCTGGCCCTGGCCCACATGATCAAGTTCCGGGGCCACTTCCTGATCGAGGGCGAC | |
| suitable for | CTGAACCCCGACAACTCCGACGTGGACAAGCTGTTCATCCAGCTGGTGCAGACCTACAACCAGCTGTTCGAGGAGAACCCCATCAA | |
| inclusion in | CGCCTCCGGCGTGGACGCCAAGGCCATCCTGTCCGCCCGGCTGTCCAAGTCCCGGCGGCTGGAGAACCTGATCGCCCAGCTGCCCG | |
| fusion | GCGAGAAGAAGAACGGCCTGTTCGGCAACCTGATCGCCCTGTCCCTGGGCCTGACCCCCAACTTCAAGTCCAACTTCGACCTGGCC | |
| protein | GAGGACGCCAAGCTGCAGCTGTCCAAGGACACCTACGACGACGACCTGGACAACCTGCTGGCCCAGATCGGCGACCAGTACGCCGA | |
| coding | CCTGTTCCTGGCCGCCAAGAACCTGTCCGACGCCATCCTGCTGTCCGACATCCTGCGGGTGAACACCGAGATCACCAAGGCCCCCC | |
| sequence) | TGTCCGCCTCCATGATCAAGCGGTACGACGAGCACCACCAGGACCTGACCCTGCTGAAGGCCCTGGTGCGGCAGCAGCTGCCCGAG | |
| | AAGTACAAGGAGATCTTCTTCGACCAGTCCAAGAACGGCTACGCCGGCTACATCGACGGCGGCGCCTCCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACGCCATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCGGCGGCTCCCCCAAGAAGAAGCGG | |
| | AAGGTG | |
| dCas9 ORF | GACAAGAAGTACTCCATCGGCCTGGCCATCGGCACCAACTCCGTGGGCTGGGCCGTGATCACCGACGAGTACAAGGTGCCCTCCAA | 353 |
| using low A | GAAGTTCAAGGTGCTGGGCAACACCGACCGGCACTCCATCAAGAAGAACCTGATCGGCGCCCTGCTGTTCGACTCCGGCGAGACCG | |
| codons of | CCGAGGCCACCCGGCTGAAGCGGACCGCCCGGCGGCGGTACACCCGGCGGAAGAACCGGATCTGCTACCTGCAGGAGATCTTCTCC | |
| Table 4 (no | AACGAGATGGCCAAGGTGGACGACTCCTTCTTCCACCGGCTGGAGGAGTCCTTCCTGGTGGAGGAGGACAAGAAGCACGAGCGGCA | |
| NLS and no | CCCCATCTTCGGCAACATCGTGGACGAGGTGGCCTACCACGAGAAGTACCCCACCATCTACCACCTGCGGAAGAAGCTGGTGGACT | |
| start or | CCACCGACAAGGCCGACCTGCGGCTGATCTACCTGGCCCTGGCCCACATGATCAAGTTCCGGGGCCACTTCCTGATCGAGGGCGAC | |
| stop codons; | CTGAACCCCGACAACTCCGACGTGGACAAGCTGTTCATCCAGCTGGTGCAGACCTACAACCAGCTGTTCGAGGAGAACCCCATCAA | |
| suitable for | CGCCTCCGGCGTGGACGCCAAGGCCATCCTGTCCGCCCGGCTGTCCAAGTCCCGGCGGCTGGAGAACCTGATCGCCCAGCTGCCCG | |
| inclusion in | GCGAGAAGAAGAACGGCCTGTTCGGCAACCTGATCGCCCTGTCCCTGGGCCTGACCCCCAACTTCAAGTCCAACTTCGACCTGGCC | |
| fusion | GAGGACGCCAAGCTGCAGCTGTCCAAGGACACCTACGACGACGACCTGGACAACCTGCTGGCCCAGATCGGCGACCAGTACGCCGA | |
| protein | CCTGTTCCTGGCCGCCAAGAACCTGTCCGACGCCATCCTGCTGTCCGACATCCTGCGGGTGAACACCGAGATCACCAAGGCCCCCC | |
| coding | TGTCCGCCTCCATGATCAAGCGGTACGACGAGCACCACCAGGACCTGACCCTGCTGAAGGCCCTGGTGCGGCAGCAGCTGCCCGAG | |
| sequence) | AAGTACAAGGAGATCTTCTTCGACCAGTCCAAGAACGGCTACGCCGGCTACATCGACGGCGGCGCCTCCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACGCCATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGAC | |
| dCas9 ORF | GACAAGAAGTACTCCATCGGCCTGGCCATCGGCACCAACTCCGTGGGCTGGGCCGTGATCACCGACGAGTACAAGGTGCCCTCCAA | 354 |
| using low A | GAAGTTCAAGGTGCTGGGCAACACCGACCGGCACTCCATCAAGAAGAACCTGATCGGCGCCCTGCTGTTCGACTCCGGCGAGACCG | |
| codons of | CCGAGGCCACCCGGCTGAAGCGGACCGCCCGGCGGCGGTACACCCGGCGGAAGAACCGGATCTGCTACCTGCAGGAGATCTTCTCC | |
| Table 4, | AACGAGATGGCCAAGGTGGACGACTCCTTCTTCCACCGGCTGGAGGAGTCCTTCCTGGTGGAGGAGGACAAGAAGCACGAGCGGCA | |
| with two C- | CCCCATCTTCGGCAACATCGTGGACGAGGTGGCCTACCACGAGAAGTACCCCACCATCTACCACCTGCGGAAGAAGCTGGTGGACT | |
| terminal NLS | CCACCGACAAGGCCGACCTGCGGCTGATCTACCTGGCCCTGGCCCACATGATCAAGTTCCGGGGCCACTTCCTGATCGAGGGCGAC | |
| sequences | CTGAACCCCGACAACTCCGACGTGGACAAGCTGTTCATCCAGCTGGTGCAGACCTACAACCAGCTGTTCGAGGAGAACCCCATCAA | |
| (no start or | CGCCTCCGGCGTGGACGCCAAGGCCATCCTGTCCGCCCGGCTGTCCAAGTCCCGGCGGCTGGAGAACCTGATCGCCCAGCTGCCCG | |
| stop codons; | GCGAGAAGAAGAACGGCCTGTTCGGCAACCTGATCGCCCTGTCCCTGGGCCTGACCCCCAACTTCAAGTCCAACTTCGACCTGGCC | |
| suitable for | GAGGACGCCAAGCTGCAGCTGTCCAAGGACACCTACGACGACGACCTGGACAACCTGCTGGCCCAGATCGGCGACCAGTACGCCGA | |
| inclusion in | CCTGTTCCTGGCCGCCAAGAACCTGTCCGACGCCATCCTGCTGTCCGACATCCTGCGGGTGAACACCGAGATCACCAAGGCCCCCC | |
| fusion | TGTCCGCCTCCATGATCAAGCGGTACGACGAGCACCACCAGGACCTGACCCTGCTGAAGGCCCTGGTGCGGCAGCAGCTGCCCGAG | |
| protein | AAGTACAAGGAGATCTTCTTCGACCAGTCCAAGAACGGCTACGCCGGCTACATCGACGGCGGCGCCTCCCAGGAGGAGTTCTACAA | |
| coding | GTTCATCAAGCCCATCCTGGAGAAGATGGACGGCACCGAGGAGCTGCTGGTGAAGCTGAACCGGGAGGACCTGCTGCGGAAGCAGC | |
| sequence) | GGACCTTCGACAACGGCTCCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACTC | |
| | CCGGTTCGCCTGGATGACCCGGAAGTCCGAGGAGACCATCACCCCCTGGAACTTCGAGGAGGTGGTGGACAAGGGCGCCTCCGCCC | |
| | AGTCCTTCATCGAGCGGATGACCAACTTCGACAAGAACCTGCCCAACGAGAAGGTGCTGCCCAAGCACTCCCTGCTGTACGAGTAC | |
| | TTCACCGTGTACAACGAGCTGACCAAGGTGAAGTACGTGACCGAGGGCATGCGGAAGCCCGCCTTCCTGTCCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACTCCGTGGAGATCTCCGGCGTGGAGGACCGGTTCAACGCCTCCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGTCCCGGAAGCTGATCAACGGCATCCGGGACAAGCAGTCCGGCAAGACCATCCTGGACTTCCTGAAGTCCGACGGCTTCGCC | |
| | AACCGGAACTTCATGCAGCTGATCCACGACGACTCCCTGACCTTCAAGGAGGACATCCAGAAGGCCCAGGTGTCCGGCCAGGGCGA | |
| | CTCCCTGCACGAGCACATCGCCAACCTGGCCGGCTCCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACTCCCGGGAGCGGATGAAGCGGATCGAGGAGGGCATCAAGGAGCTGGGCTCCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGTCCGACTACGACGTGGACGCCATCGTGCCCCAGTCCTTCCTGAAGGACGACTCCATCGACAACAAGGTGCTGACCCGGTCCGAC | |
| | AAGAACCGGGGCAAGTCCGACAACGTGCCCTCCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGTCCGAGCTGGACAAGGCCGGCTTCATCAAGC | |
| | GGCAGCTGGTGGAGACCCGGCAGATCACCAAGCACGTGGCCCAGATCCTGGACTCCCGGATGAACACCAAGTACGACGAGAACGAC | |
| | AAGCTGATCCGGGAGGTGAAGGTGATCACCCTGAAGTCCAAGCTGGTGTCCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGTCCGAGTTCGTGTACGGCGACTACAAGGTGTACGACGTGCGGAAGATGATCGCCAAGTCCGAGCAGGAGATCGGCAAGGCCACC | |
| | GCCAAGTACTTCTTCTACTCCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGTCCATGCCCC | |
| | AGGTGAACATCGTGAAGAAGACCGAGGTGCAGACCGGCGGCTTCTCCAAGGAGTCCATCCTGCCCAAGCGGAACTCCGACAAGCTG | |
| | ATCGCCCGGAAGAAGGACTGGGACCCCAAGAAGTACGGCGGCTTCGACTCCCCCACCGTGGCCTACTCCGTGCTGGTGGTGGCCAA | |
| | GGTGGAGAAGGGCAAGTCCAAGAAGCTGAAGTCCGTGAAGGAGCTGCTGGGCATCACCATCATGGAGCGGTCCTCCTTCGAGAAGA | |
| | ACCCCATCGACTTCCTGGAGGCCAAGGGCTACAAGGAGGTGAAGAAGGACCTGATCATCAAGCTGCCCAAGTACTCCCTGTTCGAG | |
| | CTGGAGAACGGCCGGAAGCGGATGCTGGCCTCCGCCGGCGAGCTGCAGAAGGGCAACGAGCTGGCCCTGCCCTCCAAGTACGTGAA | |
| | CTTCCTGTACCTGGCCTCCCACTACGAGAAGCTGAAGGGCTCCCCCGAGGACAACGAGCAGAAGCAGCTGTTCGTGGAGCAGCACA | |
| | AGCACTACCTGGACGAGATCATCGAGCAGATCTCCGAGTTCTCCAAGCGGGTGATCCTGGCCGACGCCAACCTGGACAAGGTGCTG | |
| | TCCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCTCCACCAAGGAGGTGCTGGACGCCACCCTGATCC | |
| | ACCAGTCCATCACCGGCCTGTACGAGACCCGGATCGACCTGTCCCAGCTGGGCGGCGACGGCTCCGGCTCCCCCAAGAAGAAGCGG | |
| | AAGGTGGACGGCTCCCCCAAGAAGAAGCGGAAGGTGGACTCCGGC | |
| Cas9 ORF | GACAAGAAGTACAGCATCGGCCTGGACATCGGCACCAACAGCGTGGGCTGGGCCGTGATCACCGACGAGTACAAGGTGCCCAGCAA | 355 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4, | AACGAGATGGCCAAGGTGGACGACAGCTTCTTCCACCGGCTGGAGGAGAGCTTCCTGGTGGAGGAGGACAAGAAGCACGAGCGGCA | |
| with two C- | CCCCATCTTCGGCAACATCGTGGACGAGGTGGCCTACCACGAGAAGTACCCCACCATCTACCACCTGCGGAAGAAGCTGGTGGACA | |
| terminal NLS | GCACCGACAAGGCCGACCTGCGGCTGATCTACCTGGCCCTGGCCCACATGATCAAGTTCCGGGGCCACTTCCTGATCGAGGGCGAC | |
| sequences | CTGAACCCCGACAACAGCGACGTGGACAAGCTGTTCATCCAGCTGGTGCAGACCTACAACCAGCTGTTCGAGGAGAACCCCATCAA | |
| (no start or | CGCCAGCGGCGTGGACGCCAAGGCCATCCTGAGCGCCCGGCTGAGCAAGAGCCGGCGGCTGGAGAACCTGATCGCCCAGCTGCCCG | |
| stop codons; | GCGAGAAGAAGAACGGCCTGTTCGGCAACCTGATCGCCCTGAGCCTGGGCCTGACCCCCAACTTCAAGAGCAACTTCGACCTGGCC | |
| suitable for | GAGGACGCCAAGCTGCAGCTGAGCAAGGACACCTACGACGACGACCTGGACAACCTGCTGGCCCAGATCGGCGACCAGTACGCCGA | |
| inclusion in | CCTGTTCCTGGCCGCCAAGAACCTGAGCGACGCCATCCTGCTGAGCGACATCCTGCGGGTGAACACCGAGATCACCAAGGCCCCCC | |
| fusion | TGAGCGCCAGCATGATCAAGCGGTACGACGAGCACCACCAGGACCTGACCCTGCTGAAGGCCCTGGTGCGGCAGCAGCTGCCCGAG | |
| protein | AAGTACAAGGAGATCTTCTTCGACCAGAGCAAGAACGGCTACGCCGGCTACATCGACGGCGGCGCCAGCCAGGAGGAGTTCTACAA | |
| coding | GTTCATCAAGCCCATCCTGGAGAAGATGGACGGCACCGAGGAGCTGCTGGTGAAGCTGAACCGGGAGGACCTGCTGCGGAAGCAGC | |
| sequence) | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCAGCGGCAGCCCCAAGAAGAAGCGG | |
| | AAGGTGGACGGCAGCCCCAAGAAGAAGCGGAAGGTGGACAGCGGC | |
| Cas9 ORF | GACAAGAAGTACAGCATCGGCCTGGACATCGGCACCAACAGCGTGGGCTGGGCCGTGATCACCGACGAGTACAAGGTGCCCAGCAA | 356 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4 | AACGAGATGGCCAAGGTGGACGACAGCTTCTTCCACCGGCTGGAGGAGAGCTTCCTGGTGGAGGAGGACAAGAAGCACGAGCGGCA | |
| (no NLS and | CCCCATCTTCGGCAACATCGTGGACGAGGTGGCCTACCACGAGAAGTACCCCACCATCTACCACCTGCGGAAGAAGCTGGTGGACA | |
| no start or | GCACCGACAAGGCCGACCTGCGGCTGATCTACCTGGCCCTGGCCCACATGATCAAGTTCCGGGGCCACTTCCTGATCGAGGGCGAC | |
| stop codons; | CTGAACCCCGACAACAGCGACGTGGACAAGCTGTTCATCCAGCTGGTGCAGACCTACAACCAGCTGTTCGAGGAGAACCCCATCAA | |
| suitable for | CGCCAGCGGCGTGGACGCCAAGGCCATCCTGAGCGCCCGGCTGAGCAAGAGCCGGCGGCTGGAGAACCTGATCGCCCAGCTGCCCG | |
| inclusion in | GCGAGAAGAAGAACGGCCTGTTCGGCAACCTGATCGCCCTGAGCCTGGGCCTGACCCCCAACTTCAAGAGCAACTTCGACCTGGCC | |
| fusion | GAGGACGCCAAGCTGCAGCTGAGCAAGGACACCTACGACGACGACCTGGACAACCTGCTGGCCCAGATCGGCGACCAGTACGCCGA | |
| protein | CCTGTTCCTGGCCGCCAAGAACCTGAGCGACGCCATCCTGCTGAGCGACATCCTGCGGGTGAACACCGAGATCACCAAGGCCCCCC | |
| coding | TGAGCGCCAGCATGATCAAGCGGTACGACGAGCACCACCAGGACCTGACCCTGCTGAAGGCCCTGGTGCGGCAGCAGCTGCCCGAG | |
| sequence) | AAGTACAAGGAGATCTTCTTCGACCAGAGCAAGAACGGCTACGCCGGCTACATCGACGGCGGCGCCAGCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGAC | |
| Cas9 nickase | GACAAGAAGTACAGCATCGGCCTGGCCATCGGCACCAACAGCGTGGGCTGGGCCGTGATCACCGACGAGTACAAGGTGCCCAGCAA | 357 |
| ORF using | GAAGTTCAAGGTGCTGGGCAACACCGACCGGCACAGCATCAAGAAGAACCTGATCGGCGCCCTGCTGTTCGACAGCGGCGAGACCG | |
| low A/U | CCGAGGCCACCCGGCTGAAGCGGACCGCCCGGCGGCGGTACACCCGGCGGAAGAACCGGATCTGCTACCTGCAGGAGATCTTCAGC | |
| codons of | AACGAGATGGCCAAGGTGGACGACAGCTTCTTCCACCGGCTGGAGGAGAGCTTCCTGGTGGAGGAGGACAAGAAGCACGAGCGGCA | |
| Table 4 (no | CCCCATCTTCGGCAACATCGTGGACGAGGTGGCCTACCACGAGAAGTACCCCACCATCTACCACCTGCGGAAGAAGCTGGTGGACA | |
| start or | GCACCGACAAGGCCGACCTGCGGCTGATCTACCTGGCCCTGGCCCACATGATCAAGTTCCGGGGCCACTTCCTGATCGAGGGCGAC | |
| stop codons; | CTGAACCCCGACAACAGCGACGTGGACAAGCTGTTCATCCAGCTGGTGCAGACCTACAACCAGCTGTTCGAGGAGAACCCCATCAA | |
| suitable for | CGCCAGCGGCGTGGACGCCAAGGCCATCCTGAGCGCCCGGCTGAGCAAGAGCCGGCGGCTGGAGAACCTGATCGCCCAGCTGCCCG | |
| inclusion in | GCGAGAAGAAGAACGGCCTGTTCGGCAACCTGATCGCCCTGAGCCTGGGCCTGACCCCCAACTTCAAGAGCAACTTCGACCTGGCC | |
| fusion | GAGGACGCCAAGCTGCAGCTGAGCAAGGACACCTACGACGACGACCTGGACAACCTGCTGGCCCAGATCGGCGACCAGTACGCCGA | |
| protein | CCTGTTCCTGGCCGCCAAGAACCTGAGCGACGCCATCCTGCTGAGCGACATCCTGCGGGTGAACACCGAGATCACCAAGGCCCCCC | |
| coding | TGAGCGCCAGCATGATCAAGCGGTACGACGAGCACCACCAGGACCTGACCCTGCTGAAGGCCCTGGTGCGGCAGCAGCTGCCCGAG | |
| sequence) | AAGTACAAGGAGATCTTCTTCGACCAGAGCAAGAACGGCTACGCCGGCTACATCGACGGCGGCGCCAGCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCGGCGGCAGCCCCAAGAAGAAGCGG | |
| | AAGGTG | |
| Cas9 nickase | GACAAGAAGTACAGCATCGGCCTGGCCATCGGCACCAACAGCGTGGGCTGGGCCGTGATCACCGACGAGTACAAGGTGCCCAGCAA | 358 |
| ORF using | GAAGTTCAAGGTGCTGGGCAACACCGACCGGCACAGCATCAAGAAGAACCTGATCGGCGCCCTGCTGTTCGACAGCGGCGAGACCG | |
| low A/U | CCGAGGCCACCCGGCTGAAGCGGACCGCCCGGCGGCGGTACACCCGGCGGAAGAACCGGATCTGCTACCTGCAGGAGATCTTCAGC | |
| codons of | AACGAGATGGCCAAGGTGGACGACAGCTTCTTCCACCGGCTGGAGGAGAGCTTCCTGGTGGAGGAGGACAAGAAGCACGAGCGGCA | |
| Table 4, | CCCCATCTTCGGCAACATCGTGGACGAGGTGGCCTACCACGAGAAGTACCCCACCATCTACCACCTGCGGAAGAAGCTGGTGGACA | |
| with two C- | GCACCGACAAGGCCGACCTGCGGCTGATCTACCTGGCCCTGGCCCACATGATCAAGTTCCGGGGCCACTTCCTGATCGAGGGCGAC | |
| terminal NLS | CTGAACCCCGACAACAGCGACGTGGACAAGCTGTTCATCCAGCTGGTGCAGACCTACAACCAGCTGTTCGAGGAGAACCCCATCAA | |
| sequences | CGCCAGCGGCGTGGACGCCAAGGCCATCCTGAGCGCCCGGCTGAGCAAGAGCCGGCGGCTGGAGAACCTGATCGCCCAGCTGCCCG | |
| (no start or | GCGAGAAGAAGAACGGCCTGTTCGGCAACCTGATCGCCCTGAGCCTGGGCCTGACCCCCAACTTCAAGAGCAACTTCGACCTGGCC | |
| stop codons; | GAGGACGCCAAGCTGCAGCTGAGCAAGGACACCTACGACGACGACCTGGACAACCTGCTGGCCCAGATCGGCGACCAGTACGCCGA | |
| suitable for | CCTGTTCCTGGCCGCCAAGAACCTGAGCGACGCCATCCTGCTGAGCGACATCCTGCGGGTGAACACCGAGATCACCAAGGCCCCCC | |
| inclusion in | TGAGCGCCAGCATGATCAAGCGGTACGACGAGCACCACCAGGACCTGACCCTGCTGAAGGCCCTGGTGCGGCAGCAGCTGCCCGAG | |
| fusion | AAGTACAAGGAGATCTTCTTCGACCAGAGCAAGAACGGCTACGCCGGCTACATCGACGGCGGCGCCAGCCAGGAGGAGTTCTACAA | |
| protein | GTTCATCAAGCCCATCCTGGAGAAGATGGACGGCACCGAGGAGCTGCTGGTGAAGCTGAACCGGGAGGACCTGCTGCGGAAGCAGC | |
| coding | GGACCTTCGACAACGGCAGCATCCCCCACCAGATCCACCTGGGCGAGCTGCACGCCATCCTGCGGCGGCAGGAGGACTTCTACCCC | |
| sequence) | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCAGCGGCAGCCCCAAGAAGAAGCGG | |
| | AAGGTGGACGGCAGCCCCAAGAAGAAGCGGAAGGTGGACAGCGGC | |
| Cas9 nickase | GACAAGAAGTACAGCATCGGCCTGGcCATCGGCACCAACAGCGTGGGCTGGGCCGTGATCACCGACGAGTACAAGGTGCCCAGCAA | 359 |
| ORF using | GAAGTTCAAGGTGCTGGGCAACACCGACCGGCACAGCATCAAGAAGAACCTGATCGGCGCCCTGCTGTTCGACAGCGGCGAGACCG | |
| low A/U | CCGAGGCCACCCGGCTGAAGCGGACCGCCCGGCGGCGGTACACCCGGCGGAAGAACCGGATCTGCTACCTGCAGGAGATCTTCAGC | |
| codons of | AACGAGATGGCCAAGGTGGACGACAGCTTCTTCCACCGGCTGGAGGAGAGCTTCCTGGTGGAGGAGGACAAGAAGCACGAGCGGCA | |
| Table 4 (no | CCCCATCTTCGGCAACATCGTGGACGAGGTGGCCTACCACGAGAAGTACCCCACCATCTACCACCTGCGGAAGAAGCTGGTGGACA | |
| NLS and no | GCACCGACAAGGCCGACCTGCGGCTGATCTACCTGGCCCTGGCCCACATGATCAAGTTCCGGGGCCACTTCCTGATCGAGGGCGAC | |
| start or | CTGAACCCCGACAACAGCGACGTGGACAAGCTGTTCATCCAGCTGGTGCAGACCTACAACCAGCTGTTCGAGGAGAACCCCATCAA | |
| stop codons; | CGCCAGCGGCGTGGACGCCAAGGCCATCCTGAGCGCCCGGCTGAGCAAGAGCCGGCGGCTGGAGAACCTGATCGCCCAGCTGCCCG | |
| suitable for | GCGAGAAGAAGAACGGCCTGTTCGGCAACCTGATCGCCCTGAGCCTGGGCCTGACCCCCAACTTCAAGAGCAACTTCGACCTGGCC | |
| inclusion in | GAGGACGCCAAGCTGCAGCTGAGCAAGGACACCTACGACGACGACCTGGACAACCTGCTGGCCCAGATCGGCGACCAGTACGCCGA | |
| fusion | CCTGTTCCTGGCCGCCAAGAACCTGAGCGACGCCATCCTGCTGAGCGACATCCTGCGGGTGAACACCGAGATCACCAAGGCCCCCC | |
| protein | TGAGCGCCAGCATGATCAAGCGGTACGACGAGCACCACCAGGACCTGACCCTGCTGAAGGCCCTGGTGCGGCAGCAGCTGCCCGAG | |
| coding | AAGTACAAGGAGATCTTCTTCGACCAGAGCAAGAACGGCTACGCCGGCTACATCGACGGCGGCGCCAGCCAGGAGGAGTTCTACAA | |
| sequence) | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACCACATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGAC | |
| dCas9 ORF | GACAAGAAGTACAGCATCGGCCTGGcCATCGGCACCAACAGCGTGGGCTGGGCCGTGATCACCGACGAGTACAAGGTGCCCAGCAA | 360 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4 | AACGAGATGGCCAAGGTGGACGACAGCTTCTTCCACCGGCTGGAGGAGAGCTTCCTGGTGGAGGAGGACAAGAAGCACGAGCGGCA | |
| (no start or | CCCCATCTTCGGCAACATCGTGGACGAGGTGGCCTACCACGAGAAGTACCCCACCATCTACCACCTGCGGAAGAAGCTGGTGGACA | |
| stop codons; | GCACCGACAAGGCCGACCTGCGGCTGATCTACCTGGCCCTGGCCCACATGATCAAGTTCCGGGGCCACTTCCTGATCGAGGGCGAC | |
| suitable for | CTGAACCCCGACAACAGCGACGTGGACAAGCTGTTCATCCAGCTGGTGCAGACCTACAACCAGCTGTTCGAGGAGAACCCCATCAA | |
| inclusion in | CGCCAGCGGCGTGGACGCCAAGGCCATCCTGAGCGCCCGGCTGAGCAAGAGCCGGCGGCTGGAGAACCTGATCGCCCAGCTGCCCG | |
| fusion | GCGAGAAGAAGAACGGCCTGTTCGGCAACCTGATCGCCCTGAGCCTGGGCCTGACCCCCAACTTCAAGAGCAACTTCGACCTGGCC | |
| protein | GAGGACGCCAAGCTGCAGCTGAGCAAGGACACCTACGACGACGACCTGGACAACCTGCTGGCCCAGATCGGCGACCAGTACGCCGA | |
| coding | CCTGTTCCTGGCCGCCAAGAACCTGAGCGACGCCATCCTGCTGAGCGACATCCTGCGGGTGAACACCGAGATCACCAAGGCCCCCC | |
| sequence) | TGAGCGCCAGCATGATCAAGCGGTACGACGAGCACCACCAGGACCTGACCCTGCTGAAGGCCCTGGTGCGGCAGCAGCTGCCCGAG | |
| | AAGTACAAGGAGATCTTCTTCGACCAGAGCAAGAACGGCTACGCCGGCTACATCGACGGCGGCGCCAGCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACgcCATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCGGCGGCAGCCCCAAGAAGAAGCGG | |
| | AAGGTG | |
| dCas9 ORF | GACAAGAAGTACAGCATCGGCCTGGCCATCGGCACCAACAGCGTGGGCTGGGCCGTGATCACCGACGAGTACAAGGTGCCCAGCAA | 361 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4, | AACGAGATGGCCAAGGTGGACGACAGCTTCTTCCACCGGCTGGAGGAGAGCTTCCTGGTGGAGGAGGACAAGAAGCACGAGCGGCA | |
| with two C- | CCCCATCTTCGGCAACATCGTGGACGAGGTGGCCTACCACGAGAAGTACCCCACCATCTACCACCTGCGGAAGAAGCTGGTGGACA | |
| terminal NLS | GCACCGACAAGGCCGACCTGCGGCTGATCTACCTGGCCCTGGCCCACATGATCAAGTTCCGGGGCCACTTCCTGATCGAGGGCGAC | |
| sequences | CTGAACCCCGACAACAGCGACGTGGACAAGCTGTTCATCCAGCTGGTGCAGACCTACAACCAGCTGTTCGAGGAGAACCCCATCAA | |
| (no start or | CGCCAGCGGCGTGGACGCCAAGGCCATCCTGAGCGCCCGGCTGAGCAAGAGCCGGCGGCTGGAGAACCTGATCGCCCAGCTGCCCG | |
| stop codons; | GCGAGAAGAAGAACGGCCTGTTCGGCAACCTGATCGCCCTGAGCCTGGGCCTGACCCCCAACTTCAAGAGCAACTTCGACCTGGCC | |
| suitable for | GAGGACGCCAAGCTGCAGCTGAGCAAGGACACCTACGACGACGACCTGGACAACCTGCTGGCCCAGATCGGCGACCAGTACGCCGA | |
| inclusion in | CCTGTTCCTGGCCGCCAAGAACCTGAGCGACGCCATCCTGCTGAGCGACATCCTGCGGGTGAACACCGAGATCACCAAGGCCCCCC | |
| fusion | TGAGCGCCAGCATGATCAAGCGGTACGACGAGCACCACCAGGACCTGACCCTGCTGAAGGCCCTGGTGCGGCAGCAGCTGCCCGAG | |
| protein | AAGTACAAGGAGATCTTCTTCGACCAGAGCAAGAACGGCTACGCCGGCTACATCGACGGCGGCGCCAGCCAGGAGGAGTTCTACAA | |
| coding | GTTCATCAAGCCCATCCTGGAGAAGATGGACGGCACCGAGGAGCTGCTGGTGAAGCTGAACCGGGAGGACCTGCTGCGGAAGCAGC | |
| sequence) | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACGCCATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGACGGCAGCGGCAGCCCCAAGAAGAAGCGG | |
| | AAGGTGGACGGCAGCCCCAAGAAGAAGCGGAAGGTGGACAGCGGC | |
| dCas9 ORF | GACAAGAAGTACAGCATCGGCCTGGcCATCGGCACCAACAGCGTGGGCTGGGCCGTGATCACCGACGAGTACAAGGTGCCCAGCAA | 362 |
| using low | GAAGTTCAAGGTGCTGGGCAACACCGACCGGCACAGCATCAAGAAGAACCTGATCGGCGCCCTGCTGTTCGACAGCGGCGAGACCG | |
| A/U codons | CCGAGGCCACCCGGCTGAAGCGGACCGCCCGGCGGCGGTACACCCGGCGGAAGAACCGGATCTGCTACCTGCAGGAGATCTTCAGC | |
| of Table 4 | AACGAGATGGCCAAGGTGGACGACAGCTTCTTCCACCGGCTGGAGGAGAGCTTCCTGGTGGAGGAGGACAAGAAGCACGAGCGGCA | |
| (no NLS and | CCCCATCTTCGGCAACATCGTGGACGAGGTGGCCTACCACGAGAAGTACCCCACCATCTACCACCTGCGGAAGAAGCTGGTGGACA | |
| no start or | GCACCGACAAGGCCGACCTGCGGCTGATCTACCTGGCCCTGGCCCACATGATCAAGTTCCGGGGCCACTTCCTGATCGAGGGCGAC | |
| stop codons; | CTGAACCCCGACAACAGCGACGTGGACAAGCTGTTCATCCAGCTGGTGCAGACCTACAACCAGCTGTTCGAGGAGAACCCCATCAA | |
| suitable for | CGCCAGCGGCGTGGACGCCAAGGCCATCCTGAGCGCCCGGCTGAGCAAGAGCCGGCGGCTGGAGAACCTGATCGCCCAGCTGCCCG | |
| inclusion in | GCGAGAAGAAGAACGGCCTGTTCGGCAACCTGATCGCCCTGAGCCTGGGCCTGACCCCCAACTTCAAGAGCAACTTCGACCTGGCC | |
| fusion | GAGGACGCCAAGCTGCAGCTGAGCAAGGACACCTACGACGACGACCTGGACAACCTGCTGGCCCAGATCGGCGACCAGTACGCCGA | |
| protein | CCTGTTCCTGGCCGCCAAGAACCTGAGCGACGCCATCCTGCTGAGCGACATCCTGCGGGTGAACACCGAGATCACCAAGGCCCCCC | |
| coding | TGAGCGCCAGCATGATCAAGCGGTACGACGAGCACCACCAGGACCTGACCCTGCTGAAGGCCCTGGTGCGGCAGCAGCTGCCCGAG | |
| sequence) | AAGTACAAGGAGATCTTCTTCGACCAGAGCAAGAACGGCTACGCCGGCTACATCGACGGCGGCGCCAGCCAGGAGGAGTTCTACAA | |
| | GTTCATCAAGCCCATCCTGGAGAAGATGGACGGCACCGAGGAGCTGCTGGTGAAGCTGAACCGGGAGGACCTGCTGCGGAAGCAGC | |
| | GGACCTTCGACAACGGCAGCATCCCCCACCAGATCCACCTGGGCGAGCTGCACGCCATCCTGCGGCGGCAGGAGGACTTCTACCCC | |
| | TTCCTGAAGGACAACCGGGAGAAGATCGAGAAGATCCTGACCTTCCGGATCCCCTACTACGTGGGCCCCCTGGCCCGGGGCAACAG | |
| | CCGGTTCGCCTGGATGACCCGGAAGAGCGAGGAGACCATCACCCCCTGGAACTTCGAGGAGGTGGTGGACAAGGGCGCCAGCGCCC | |
| | AGAGCTTCATCGAGCGGATGACCAACTTCGACAAGAACCTGCCCAACGAGAAGGTGCTGCCCAAGCACAGCCTGCTGTACGAGTAC | |
| | TTCACCGTGTACAACGAGCTGACCAAGGTGAAGTACGTGACCGAGGGCATGCGGAAGCCCGCCTTCCTGAGCGGCGAGCAGAAGAA | |
| | GGCCATCGTGGACCTGCTGTTCAAGACCAACCGGAAGGTGACCGTGAAGCAGCTGAAGGAGGACTACTTCAAGAAGATCGAGTGCT | |
| | TCGACAGCGTGGAGATCAGCGGCGTGGAGGACCGGTTCAACGCCAGCCTGGGCACCTACCACGACCTGCTGAAGATCATCAAGGAC | |
| | AAGGACTTCCTGGACAACGAGGAGAACGAGGACATCCTGGAGGACATCGTGCTGACCCTGACCCTGTTCGAGGACCGGGAGATGAT | |
| | CGAGGAGCGGCTGAAGACCTACGCCCACCTGTTCGACGACAAGGTGATGAAGCAGCTGAAGCGGCGGCGGTACACCGGCTGGGGCC | |
| | GGCTGAGCCGGAAGCTGATCAACGGCATCCGGGACAAGCAGAGCGGCAAGACCATCCTGGACTTCCTGAAGAGCGACGGCTTCGCC | |
| | AACCGGAACTTCATGCAGCTGATCCACGACGACAGCCTGACCTTCAAGGAGGACATCCAGAAGGCCCAGGTGAGCGGCCAGGGCGA | |
| | CAGCCTGCACGAGCACATCGCCAACCTGGCCGGCAGCCCCGCCATCAAGAAGGGCATCCTGCAGACCGTGAAGGTGGTGGACGAGC | |
| | TGGTGAAGGTGATGGGCCGGCACAAGCCCGAGAACATCGTGATCGAGATGGCCCGGGAGAACCAGACCACCCAGAAGGGCCAGAAG | |
| | AACAGCCGGGAGCGGATGAAGCGGATCGAGGAGGGCATCAAGGAGCTGGGCAGCCAGATCCTGAAGGAGCACCCCGTGGAGAACAC | |
| | CCAGCTGCAGAACGAGAAGCTGTACCTGTACTACCTGCAGAACGGCCGGGACATGTACGTGGACCAGGAGCTGGACATCAACCGGC | |
| | TGAGCGACTACGACGTGGACgcCATCGTGCCCCAGAGCTTCCTGAAGGACGACAGCATCGACAACAAGGTGCTGACCCGGAGCGAC | |
| | AAGAACCGGGGCAAGAGCGACAACGTGCCCAGCGAGGAGGTGGTGAAGAAGATGAAGAACTACTGGCGGCAGCTGCTGAACGCCAA | |
| | GCTGATCACCCAGCGGAAGTTCGACAACCTGACCAAGGCCGAGCGGGGCGGCCTGAGCGAGCTGGACAAGGCCGGCTTCATCAAGC | |
| | GGCAGCTGGTGGAGACCCGGCAGATCACCAAGCACGTGGCCCAGATCCTGGACAGCCGGATGAACACCAAGTACGACGAGAACGAC | |
| | AAGCTGATCCGGGAGGTGAAGGTGATCACCCTGAAGAGCAAGCTGGTGAGCGACTTCCGGAAGGACTTCCAGTTCTACAAGGTGCG | |
| | GGAGATCAACAACTACCACCACGCCCACGACGCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGCCAAGAGCGAGCAGGAGATCGGCAAGGCCACC | |
| | GCCAAGTACTTCTTCTACAGCAACATCATGAACTTCTTCAAGACCGAGATCACCCTGGCCAACGGCGAGATCCGGAAGCGGCCCCT | |
| | GATCGAGACCAACGGCGAGACCGGCGAGATCGTGTGGGACAAGGGCCGGGACTTCGCCACCGTGCGGAAGGTGCTGAGCATGCCCC | |
| | AGGTGAACATCGTGAAGAAGACCGAGGTGCAGACCGGCGGCTTCAGCAAGGAGAGCATCCTGCCCAAGCGGAACAGCGACAAGCTG | |
| | ATCGCCCGGAAGAAGGACTGGGACCCCAAGAAGTACGGCGGCTTCGACAGCCCCACCGTGGCCTACAGCGTGCTGGTGGTGGCCAA | |
| | GGTGGAGAAGGGCAAGAGCAAGAAGCTGAAGAGCGTGAAGGAGCTGCTGGGCATCACCATCATGGAGCGGAGCAGCTTCGAGAAGA | |
| | ACCCCATCGACTTCCTGGAGGCCAAGGGCTACAAGGAGGTGAAGAAGGACCTGATCATCAAGCTGCCCAAGTACAGCCTGTTCGAG | |
| | CTGGAGAACGGCCGGAAGCGGATGCTGGCCAGCGCCGGCGAGCTGCAGAAGGGCAACGAGCTGGCCCTGCCCAGCAAGTACGTGAA | |
| | CTTCCTGTACCTGGCCAGCCACTACGAGAAGCTGAAGGGCAGCCCCGAGGACAACGAGCAGAAGCAGCTGTTCGTGGAGCAGCACA | |
| | AGCACTACCTGGACGAGATCATCGAGCAGATCAGCGAGTTCAGCAAGCGGGTGATCCTGGCCGACGCCAACCTGGACAAGGTGCTG | |
| | AGCGCCTACAACAAGCACCGGGACAAGCCCATCCGGGAGCAGGCCGAGAACATCATCCACCTGTTCACCCTGACCAACCTGGGCGC | |
| | CCCCGCCGCCTTCAAGTACTTCGACACCACCATCGACCGGAAGCGGTACACCAGCACCAAGGAGGTGCTGGACGCCACCCTGATCC | |
| | ACCAGAGCATCACCGGCCTGTACGAGACCCGGATCGACCTGAGCCAGCTGGGCGGCGAC | |
| Nme Cas9 ORF | GCCGCCTTCAAGCCCAACTCCATCAACTACATCCTGGGCCTGGACATCGGCATCGCCTCCGTGGGCTGGGCCATGGTGGAGATCGA | 363 |
| using low A | CGAGGAGGAGAACCCCATCCGGCTGATCGACCTGGGCGTGCGGGTGTTCGAGCGGGCCGAGGTGCCCAAGACCGGCGACTCCCTGG | |
| codons of | CCATGGCCCGGCGGCTGGCCCGGTCCGTGCGGCGGCTGACCCGGCGGCGGGCCCACCGGCTGCTGCGGACCCGGCGGCTGCTGAAG | |
| Table 4 (no | CGGGAGGGCGTGCTGCAGGCCGCCAACTTCGACGAGAACGGCCTGATCAAGTCCCTGCCCAACACCCCCTGGCAGCTGCGGGCCGC | |
| start or | CGCCCTGGACCGGAAGCTGACCCCCCTGGAGTGGTCCGCCGTGCTGCTGCACCTGATCAAGCACCGGGGCTACCTGTCCCAGCGGA | |
| stop codons; | AGAACGAGGGCGAGACCGCCGACAAGGAGCTGGGCGCCCTGCTGAAGGGCGTGGCCGGCAACGCCCACGCCCTGCAGACCGGCGAC | |
| suitable for | TTCCGGACCCCCGCCGAGCTGGCCCTGAACAAGTTCGAGAAGGAGTCCGGCCACATCCGGAACCAGCGGTCCGACTACTCCCACAC | |
| inclusion in | CTTCTCCCGGAAGGACCTGCAGGCCGAGCTGATCCTGCTGTTCGAGAAGCAGAAGGAGTTCGGCAACCCCCACGTGTCCGGCGGCC | |
| fusion | TGAAGGAGGGCATCGAGACCCTGCTGATGACCCAGCGGCCCGCCCTGTCCGGCGACGCCGTGCAGAAGATGCTGGGCCACTGCACC | |
| protein | TTCGAGCCCGCCGAGCCCAAGGCCGCCAAGAACACCTACACCGCCGAGCGGTTCATCTGGCTGACCAAGCTGAACAACCTGCGGAT | |
| coding | CCTGGAGCAGGGCTCCGAGCGGCCCCTGACCGACACCGAGCGGGCCACCCTGATGGACGAGCCCTACCGGAAGTCCAAGCTGACCT | |
| sequence) | ACGCCCAGGCCCGGAAGCTGCTGGGCCTGGAGGACACCGCCTTCTTCAAGGGCCTGCGGTACGGCAAGGACAACGCCGAGGCCTCC | |
| | ACCCTGATGGAGATGAAGGCCTACCACGCCATCTCCCGGGCCCTGGAGAAGGAGGGCCTGAAGGACAAGAAGTCCCCCCTGAACCT | |
| | GTCCCCCGAGCTGCAGGACGAGATCGGCACCGCCTTCTCCCTGTTCAAGACCGACGAGGACATCACCGGCCGGCTGAAGGACCGGA | |
| | TCCAGCCCGAGATCCTGGAGGCCCTGCTGAAGCACATCTCCTTCGACAAGTTCGTGCAGATCTCCCTGAAGGCCCTGCGGCGGATC | |
| | GTGCCCCTGATGGAGCAGGGCAAGCGGTACGACGAGGCCTGCGCCGAGATCTACGGCGACCACTACGGCAAGAAGAACACCGAGGA | |
| | GAAGATCTACCTGCCCCCCATCCCCGCCGACGAGATCCGGAACCCCGTGGTGCTGCGGGCCCTGTCCCAGGCCCGGAAGGTGATCA | |
| | ACGGCGTGGTGCGGCGGTACGGCTCCCCCGCCCGGATCCACATCGAGACCGCCCGGGAGGTGGGCAAGTCCTTCAAGGACCGGAAG | |
| | GAGATCGAGAAGCGGCAGGAGGAGAACCGGAAGGACCGGGAGAAGGCCGCCGCCAAGTTCCGGGAGTACTTCCCCAACTTCGTGGG | |
| | CGAGCCCAAGTCCAAGGACATCCTGAAGCTGCGGCTGTACGAGCAGCAGCACGGCAAGTGCCTGTACTCCGGCAAGGAGATCAACC | |
| | TGGGCCGGCTGAACGAGAAGGGCTACGTGGAGATCGACCACGCCCTGCCCTTCTCCCGGACCTGGGACGACTCCTTCAACAACAAG | |
| | GTGCTGGTGCTGGGCTCCGAGAACCAGAACAAGGGCAACCAGACCCCCTACGAGTACTTCAACGGCAAGGACAACTCCCGGGAGTG | |
| | GCAGGAGTTCAAGGCCCGGGTGGAGACCTCCCGGTTCCCCCGGTCCAAGAAGCAGCGGATCCTGCTGCAGAAGTTCGACGAGGACG | |
| | GCTTCAAGGAGCGGAACCTGAACGACACCCGGTACGTGAACCGGTTCCTGTGCCAGTTCGTGGCCGACCGGATGCGGCTGACCGGC | |
| | AAGGGCAAGAAGCGGGTGTTCGCCTCCAACGGCCAGATCACCAACCTGCTGCGGGGCTTCTGGGGCCTGCGGAAGGTGCGGGCCGA | |
| | GAACGACCGGCACCACGCCCTGGACGCCGTGGTGGTGGCCTGCTCCACCGTGGCCATGCAGCAGAAGATCACCCGGTTCGTGCGGT | |
| | ACAAGGAGATGAACGCCTTCGACGGCAAGACCATCGACAAGGAGACCGGCGAGGTGCTGCACCAGAAGACCCACTTCCCCCAGCCC | |
| | TGGGAGTTCTTCGCCCAGGAGGTGATGATCCGGGTGTTCGGCAAGCCCGACGGCAAGCCCGAGTTCGAGGAGGCCGACACCCTGGA | |
| | GAAGCTGCGGACCCTGCTGGCCGAGAAGCTGTCCTCCCGGCCCGAGGCCGTGCACGAGTACGTGACCCCCCTGTTCGTGTCCCGGG | |
| | CCCCCAACCGGAAGATGTCCGGCCAGGGCCACATGGAGACCGTGAAGTCCGCCAAGCGGCTGGACGAGGGCGTGTCCGTGCTGCGG | |
| | GTGCCCCTGACCCAGCTGAAGCTGAAGGACCTGGAGAAGATGGTGAACCGGGAGCGGGAGCCCAAGCTGTACGAGGCCCTGAAGGC | |
| | CCGGCTGGAGGCCCACAAGGACGACCCCGCCAAGGCCTTCGCCGAGCCCTTCTACAAGTACGACAAGGCCGGCAACCGGACCCAGC | |
| | AGGTGAAGGCCGTGCGGGTGGAGCAGGTGCAGAAGACCGGCGTGTGGGTGCGGAACCACAACGGCATCGCCGACAACGCCACCATG | |
| | GTGCGGGTGGACGTGTTCGAGAAGGGCGACAAGTACTACCTGGTGCCCATCTACTCCTGGCAGGTGGCCAAGGGCATCCTGCCCGA | |
| | CCGGGCCGTGGTGCAGGGCAAGGACGAGGAGGACTGGCAGCTGATCGACGACTCCTTCAACTTCAAGTTCTCCCTGCACCCCAACG | |
| | ACCTGGTGGAGGTGATCACCAAGAAGGCCCGGATGTTCGGCTACTTCGCCTCCTGCCACCGGGGCACCGGCAACATCAACATCCGG | |
| | ATCCACGACCTGGACCACAAGATCGGCAAGAACGGCATCCTGGAGGGCATCGGCGTGAAGACCGCCCTGTCCTTCCAGAAGTACCA | |
| | GATCGACGAGCTGGGCAAGGAGATCCGGCCCTGCCGGCTGAAGAAGCGGCCCCCCGTGCGGTCCGGCAAGCGGACCGCCGACGGCT | |
| | CCGAGTTCGAGTCCCCCAAGAAGAAGCGGAAGGTGGAG | |
| Nme Cas9 ORF | GCCGCCTTCAAGCCCAACAGCATCAACTACATCCTGGGCCTGGACATCGGCATCGCCAGCGTGGGCTGGGCCATGGTGGAGATCGA | 364 |
| using low | CGAGGAGGAGAACCCCATCCGGCTGATCGACCTGGGCGTGCGGGTGTTCGAGCGGGCCGAGGTGCCCAAGACCGGCGACAGCCTGG | |
| A/U codons | CCATGGCCCGGCGGCTGGCCCGGAGCGTGCGGCGGCTGACCCGGCGGCGGGCCCACCGGCTGCTGCGGACCCGGCGGCTGCTGAAG | |
| of Table 4 | CGGGAGGGCGTGCTGCAGGCCGCCAACTTCGACGAGAACGGCCTGATCAAGAGCCTGCCCAACACCCCCTGGCAGCTGCGGGCCGC | |
| (no start or | CGCCCTGGACCGGAAGCTGACCCCCCTGGAGTGGAGCGCCGTGCTGCTGCACCTGATCAAGCACCGGGGCTACCTGAGCCAGCGGA | |
| stop codons; | AGAACGAGGGCGAGACCGCCGACAAGGAGCTGGGCGCCCTGCTGAAGGGCGTGGCCGGCAACGCCCACGCCCTGCAGACCGGCGAC | |
| suitable for | TTCCGGACCCCCGCCGAGCTGGCCCTGAACAAGTTCGAGAAGGAGAGCGGCCACATCCGGAACCAGCGGAGCGACTACAGCCACAC | |
| inclusion in | CTTCAGCCGGAAGGACCTGCAGGCCGAGCTGATCCTGCTGTTCGAGAAGCAGAAGGAGTTCGGCAACCCCCACGTGAGCGGCGGCC | |
| fusion | TGAAGGAGGGCATCGAGACCCTGCTGATGACCCAGCGGCCCGCCCTGAGCGGCGACGCCGTGCAGAAGATGCTGGGCCACTGCACC | |
| protein | TTCGAGCCCGCCGAGCCCAAGGCCGCCAAGAACACCTACACCGCCGAGCGGTTCATCTGGCTGACCAAGCTGAACAACCTGCGGAT | |
| coding | CCTGGAGCAGGGCAGCGAGCGGCCCCTGACCGACACCGAGCGGGCCACCCTGATGGACGAGCCCTACCGGAAGAGCAAGCTGACCT | |
| sequence) | ACGCCCAGGCCCGGAAGCTGCTGGGCCTGGAGGACACCGCCTTCTTCAAGGGCCTGCGGTACGGCAAGGACAACGCCGAGGCCAGC | |
| | ACCCTGATGGAGATGAAGGCCTACCACGCCATCAGCCGGGCCCTGGAGAAGGAGGGCCTGAAGGACAAGAAGAGCCCCCTGAACCT | |
| | GAGCCCCGAGCTGCAGGACGAGATCGGCACCGCCTTCAGCCTGTTCAAGACCGACGAGGACATCACCGGCCGGCTGAAGGACCGGA | |
| | TCCAGCCCGAGATCCTGGAGGCCCTGCTGAAGCACATCAGCTTCGACAAGTTCGTGCAGATCAGCCTGAAGGCCCTGCGGCGGATC | |
| | GTGCCCCTGATGGAGCAGGGCAAGCGGTACGACGAGGCCTGCGCCGAGATCTACGGCGACCACTACGGCAAGAAGAACACCGAGGA | |
| | GAAGATCTACCTGCCCCCCATCCCCGCCGACGAGATCCGGAACCCCGTGGTGCTGCGGGCCCTGAGCCAGGCCCGGAAGGTGATCA | |
| | ACGGCGTGGTGCGGCGGTACGGCAGCCCCGCCCGGATCCACATCGAGACCGCCCGGGAGGTGGGCAAGAGCTTCAAGGACCGGAAG | |
| | GAGATCGAGAAGCGGCAGGAGGAGAACCGGAAGGACCGGGAGAAGGCCGCCGCCAAGTTCCGGGAGTACTTCCCCAACTTCGTGGG | |
| | CGAGCCCAAGAGCAAGGACATCCTGAAGCTGCGGCTGTACGAGCAGCAGCACGGCAAGTGCCTGTACAGCGGCAAGGAGATCAACC | |
| | TGGGCCGGCTGAACGAGAAGGGCTACGTGGAGATCGACCACGCCCTGCCCTTCAGCCGGACCTGGGACGACAGCTTCAACAACAAG | |
| | GTGCTGGTGCTGGGCAGCGAGAACCAGAACAAGGGCAACCAGACCCCCTACGAGTACTTCAACGGCAAGGACAACAGCCGGGAGTG | |
| | GCAGGAGTTCAAGGCCCGGGTGGAGACCAGCCGGTTCCCCCGGAGCAAGAAGCAGCGGATCCTGCTGCAGAAGTTCGACGAGGACG | |
| | GCTTCAAGGAGCGGAACCTGAACGACACCCGGTACGTGAACCGGTTCCTGTGCCAGTTCGTGGCCGACCGGATGCGGCTGACCGGC | |
| | AAGGGCAAGAAGCGGGTGTTCGCCAGCAACGGCCAGATCACCAACCTGCTGCGGGGCTTCTGGGGCCTGCGGAAGGTGCGGGCCGA | |
| | GAACGACCGGCACCACGCCCTGGACGCCGTGGTGGTGGCCTGCAGCACCGTGGCCATGCAGCAGAAGATCACCCGGTTCGTGCGGT | |
| | ACAAGGAGATGAACGCCTTCGACGGCAAGACCATCGACAAGGAGACCGGCGAGGTGCTGCACCAGAAGACCCACTTCCCCCAGCCC | |
| | TGGGAGTTCTTCGCCCAGGAGGTGATGATCCGGGTGTTCGGCAAGCCCGACGGCAAGCCCGAGTTCGAGGAGGCCGACACCCTGGA | |
| | GAAGCTGCGGACCCTGCTGGCCGAGAAGCTGAGCAGCCGGCCCGAGGCCGTGCACGAGTACGTGACCCCCCTGTTCGTGAGCCGGG | |
| | CCCCCAACCGGAAGATGAGCGGCCAGGGCCACATGGAGACCGTGAAGAGCGCCAAGCGGCTGGACGAGGGCGTGAGCGTGCTGCGG | |
| | GTGCCCCTGACCCAGCTGAAGCTGAAGGACCTGGAGAAGATGGTGAACCGGGAGCGGGAGCCCAAGCTGTACGAGGCCCTGAAGGC | |
| | CCGGCTGGAGGCCCACAAGGACGACCCCGCCAAGGCCTTCGCCGAGCCCTTCTACAAGTACGACAAGGCCGGCAACCGGACCCAGC | |
| | AGGTGAAGGCCGTGCGGGTGGAGCAGGTGCAGAAGACCGGCGTGTGGGTGCGGAACCACAACGGCATCGCCGACAACGCCACCATG | |
| | GTGCGGGTGGACGTGTTCGAGAAGGGCGACAAGTACTACCTGGTGCCCATCTACAGCTGGCAGGTGGCCAAGGGCATCCTGCCCGA | |
| | CCGGGCCGTGGTGCAGGGCAAGGACGAGGAGGACTGGCAGCTGATCGACGACAGCTTCAACTTCAAGTTCAGCCTGCACCCCAACG | |
| | ACCTGGTGGAGGTGATCACCAAGAAGGCCCGGATGTTCGGCTACTTCGCCAGCTGCCACCGGGGCACCGGCAACATCAACATCCGG | |
| | ATCCACGACCTGGACCACAAGATCGGCAAGAACGGCATCCTGGAGGGCATCGGCGTGAAGACCGCCCTGAGCTTCCAGAAGTACCA | |
| | GATCGACGAGCTGGGCAAGGAGATCCGGCCCTGCCGGCTGAAGAAGCGGCCCCCCGTGCGGAGCGGCAAGCGGACCGCCGACGGCA | |
| | GCGAGTTCGAGAGCCCCAAGAAGAAGCGGAAGGTGGAG | |
| Open reading | GACAAGAAGTACAGCATCGGACTGGACATCGGAACAAACAGCGTCGGATGGGCAGTCATCACAGACGAATACAAGGTCCCGAGCAA | 365 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS1 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCCTGGCAGCAAAGAGA | |
| | AGCAGAACAACA | |
| Open reading | GACAAGAAGTACAGCATCGGACTGGACATCGGAACAAACAGCGTCGGATGGGCAGTCATCACAGACGAATACAAGGTCCCGAGCAA | 366 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS2 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCCAGGCAGCAAAGAGA | |
| | AGCAGAACAACA | |
| Open reading | GACAAGAAGTACAGCATCGGACTGGACATCGGAACAAACAGCGTCGGATGGGCAGTCATCACAGACGAATACAAGGTCCCGAGCAA | 367 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS3 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCCCGGCACCGGCAAAG | |
| | AGAGAAAGAACAACA | |
| Open reading | GACAAGAAGTACAGCATCGGACTGGACATCGGAACAAACAGCGTCGGATGGGCAGTCATCACAGACGAATACAAGGTCCCGAGCAA | 368 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS4 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCCAGGCAGCAAAGAGA | |
| | CCGAGAACAACA | |
| Open reading | GACAAGAAGTACAGCATCGGACTGGACATCGGAACAAACAGCGTCGGATGGGCAGTCATCACAGACGAATACAAGGTCCCGAGCAA | 369 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS5 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCAGAGCAGCAAAGAGA | |
| | CCGAGAACAACA | |
| Open reading | GACAAGAAGTACAGCATCGGACTGGACATCGGAACAAACAGCGTCGGATGGGCAGTCATCACAGACGAATACAAGGTCCCGAGCAA | 370 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS6 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCGCAGCAGCAAAGAGA | |
| | AGCTGGAGCATGGCAGCA | |
| Open reading | GACAAGAAGTACAGCATCGGACTGGACATCGGAACAAACAGCGTCGGATGGGCAGTCATCACAGACGAATACAAGGTCCCGAGCAA | 371 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS7 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCGCAGCAGCAAAGAGA | |
| | GTCTGGAGCATGGCATTC | |
| Open reading | GACAAGAAGTACAGCATCGGACTGGACATCGGAACAAACAGCGTCGGATGGGCAGTCATCACAGACGAATACAAGGTCCCGAGCAA | 372 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS8 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCGCAGCAGCAAAGAGA | |
| | AGCTGGAGCATGGCATTC | |
| Open reading | GACAAGAAGTACAGCATCGGACTGGACATCGGAACAAACAGCGTCGGATGGGCAGTCATCACAGACGAATACAAGGTCCCGAGCAA | 373 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS9 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCGCAGCAGCAAAGAGA | |
| | AAGTACTTCGCAGCA | |
| Open reading | GACAAGAAGTACAGCATCGGACTGGACATCGGAACAAACAGCGTCGGATGGGCAGTCATCACAGACGAATACAAGGTCCCGAGCAA | 374 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS10 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCAGAGCAGCAAAGAGA | |
| | AAGGCATTCGCAGCA | |
| Open reading | GACAAGAAGTACAGCATCGGACTGGACATCGGAACAAACAGCGTCGGATGGGCAGTCATCACAGACGAATACAAGGTCCCGAGCAA | 375 |
| frame for | GAAGTTCAAGGTCCTGGGAAACACAGACAGACACAGCATCAAGAAGAACCTGATCGGAGCACTGCTGTTCGACAGCGGAGAAACAG | |
| Cas9 with | CAGAAGCAACAAGACTGAAGAGAACAGCAAGAAGAAGATACACAAGAAGAAAGAACAGAATCTGCTACCTGCAGGAAATCTTCAGC | |
| NLS11 (no | AACGAAATGGCAAAGGTCGACGACAGCTTCTTCCACAGACTGGAAGAAAGCTTCCTGGTCGAAGAAGACAAGAAGCACGAAAGACA | |
| start or | CCCGATCTTCGGAAACATCGTCGACGAAGTCGCATACCACGAAAAGTACCCGACAATCTACCACCTGAGAAAGAAGCTGGTCGACA | |
| stop codons; | GCACAGACAAGGCAGACCTGAGACTGATCTACCTGGCACTGGCACACATGATCAAGTTCAGAGGACACTTCCTGATCGAAGGAGAC | |
| suitable for | CTGAACCCGGACAACAGCGACGTCGACAAGCTGTTCATCCAGCTGGTCCAGACATACAACCAGCTGTTCGAAGAAAACCCGATCAA | |
| inclusion in | CGCAAGCGGAGTCGACGCAAAGGCAATCCTGAGCGCAAGACTGAGCAAGAGCAGAAGACTGGAAAACCTGATCGCACAGCTGCCGG | |
| fusion | GAGAAAAGAAGAACGGACTGTTCGGAAACCTGATCGCACTGAGCCTGGGACTGACACCGAACTTCAAGAGCAACTTCGACCTGGCA | |
| protein | GAAGACGCAAAGCTGCAGCTGAGCAAGGACACATACGACGACGACCTGGACAACCTGCTGGCACAGATCGGAGACCAGTACGCAGA | |
| coding | CCTGTTCCTGGCAGCAAAGAACCTGAGCGACGCAATCCTGCTGAGCGACATCCTGAGAGTCAACACAGAAATCACAAAGGCACCGC | |
| sequence) | TGAGCGCAAGCATGATCAAGAGATACGACGAACACCACCAGGACCTGACACTGCTGAAGGCACTGGTCAGACAGCAGCTGCCGGAA | |
| | AAGTACAAGGAAATCTTCTTCGACCAGAGCAAGAACGGATACGCAGGATACATCGACGGAGGAGCAAGCCAGGAAGAATTCTACAA | |
| | GTTCATCAAGCCGATCCTGGAAAAGATGGACGGAACAGAAGAACTGCTGGTCAAGCTGAACAGAGAAGACCTGCTGAGAAAGCAGA | |
| | GAACATTCGACAACGGAAGCATCCCGCACCAGATCCACCTGGGAGAACTGCACGCAATCCTGAGAAGACAGGAAGACTTCTACCCG | |
| | TTCCTGAAGGACAACAGAGAAAAGATCGAAAAGATCCTGACATTCAGAATCCCGTACTACGTCGGACCGCTGGCAAGAGGAAACAG | |
| | CAGATTCGCATGGATGACAAGAAAGAGCGAAGAAACAATCACACCGTGGAACTTCGAAGAAGTCGTCGACAAGGGAGCAAGCGCAC | |
| | AGAGCTTCATCGAAAGAATGACAAACTTCGACAAGAACCTGCCGAACGAAAAGGTCCTGCCGAAGCACAGCCTGCTGTACGAATAC | |
| | TTCACAGTCTACAACGAACTGACAAAGGTCAAGTACGTCACAGAAGGAATGAGAAAGCCGGCATTCCTGAGCGGAGAACAGAAGAA | |
| | GGCAATCGTCGACCTGCTGTTCAAGACAAACAGAAAGGTCACAGTCAAGCAGCTGAAGGAAGACTACTTCAAGAAGATCGAATGCT | |
| | TCGACAGCGTCGAAATCAGCGGAGTCGAAGACAGATTCAACGCAAGCCTGGGAACATACCACGACCTGCTGAAGATCATCAAGGAC | |
| | AAGGACTTCCTGGACAACGAAGAAAACGAAGACATCCTGGAAGACATCGTCCTGACACTGACACTGTTCGAAGACAGAGAAATGAT | |
| | CGAAGAAAGACTGAAGACATACGCACACCTGTTCGACGACAAGGTCATGAAGCAGCTGAAGAGAAGAAGATACACAGGATGGGGAA | |
| | GACTGAGCAGAAAGCTGATCAACGGAATCAGAGACAAGCAGAGCGGAAAGACAATCCTGGACTTCCTGAAGAGCGACGGATTCGCA | |
| | AACAGAAACTTCATGCAGCTGATCCACGACGACAGCCTGACATTCAAGGAAGACATCCAGAAGGCACAGGTCAGCGGACAGGGAGA | |
| | CAGCCTGCACGAACACATCGCAAACCTGGCAGGAAGCCCGGCAATCAAGAAGGGAATCCTGCAGACAGTCAAGGTCGTCGACGAAC | |
| | TGGTCAAGGTCATGGGAAGACACAAGCCGGAAAACATCGTCATCGAAATGGCAAGAGAAAACCAGACAACACAGAAGGGACAGAAG | |
| | AACAGCAGAGAAAGAATGAAGAGAATCGAAGAAGGAATCAAGGAACTGGGAAGCCAGATCCTGAAGGAACACCCGGTCGAAAACAC | |
| | ACAGCTGCAGAACGAAAAGCTGTACCTGTACTACCTGCAGAACGGAAGAGACATGTACGTCGACCAGGAACTGGACATCAACAGAC | |
| | TGAGCGACTACGACGTCGACCACATCGTCCCGCAGAGCTTCCTGAAGGACGACAGCATCGACAACAAGGTCCTGACAAGAAGCGAC | |
| | AAGAACAGAGGAAAGAGCGACAACGTCCCGAGCGAAGAAGTCGTCAAGAAGATGAAGAACTACTGGAGACAGCTGCTGAACGCAAA | |
| | GCTGATCACACAGAGAAAGTTCGACAACCTGACAAAGGCAGAGAGAGGAGGACTGAGCGAACTGGACAAGGCAGGATTCATCAAGA | |
| | GACAGCTGGTCGAAACAAGACAGATCACAAAGCACGTCGCACAGATCCTGGACAGCAGAATGAACACAAAGTACGACGAAAACGAC | |
| | AAGCTGATCAGAGAAGTCAAGGTCATCACACTGAAGAGCAAGCTGGTCAGCGACTTCAGAAAGGACTTCCAGTTCTACAAGGTCAG | |
| | AGAAATCAACAACTACCACCACGCACACGACGCATACCTGAACGCAGTCGTCGGAACAGCACTGATCAAGAAGTACCCGAAGCTGG | |
| | AAAGCGAATTCGTCTACGGAGACTACAAGGTCTACGACGTCAGAAAGATGATCGCAAAGAGCGAACAGGAAATCGGAAAGGCAACA | |
| | GCAAAGTACTTCTTCTACAGCAACATCATGAACTTCTTCAAGACAGAAATCACACTGGCAAACGGAGAAATCAGAAAGAGACCGCT | |
| | GATCGAAACAAACGGAGAAACAGGAGAAATCGTCTGGGACAAGGGAAGAGACTTCGCAACAGTCAGAAAGGTCCTGAGCATGCCGC | |
| | AGGTCAACATCGTCAAGAAGACAGAAGTCCAGACAGGAGGATTCAGCAAGGAAAGCATCCTGCCGAAGAGAAACAGCGACAAGCTG | |
| | ATCGCAAGAAAGAAGGACTGGGACCCGAAGAAGTACGGAGGATTCGACAGCCCGACAGTCGCATACAGCGTCCTGGTCGTCGCAAA | |
| | GGTCGAAAAGGGAAAGAGCAAGAAGCTGAAGAGCGTCAAGGAACTGCTGGGAATCACAATCATGGAAAGAAGCAGCTTCGAAAAGA | |
| | ACCCGATCGACTTCCTGGAAGCAAAGGGATACAAGGAAGTCAAGAAGGACCTGATCATCAAGCTGCCGAAGTACAGCCTGTTCGAA | |
| | CTGGAAAACGGAAGAAAGAGAATGCTGGCAAGCGCAGGAGAACTGCAGAAGGGAAACGAACTGGCACTGCCGAGCAAGTACGTCAA | |
| | CTTCCTGTACCTGGCAAGCCACTACGAAAAGCTGAAGGGAAGCCCGGAAGACAACGAACAGAAGCAGCTGTTCGTCGAACAGCACA | |
| | AGCACTACCTGGACGAAATCATCGAACAGATCAGCGAATTCAGCAAGAGAGTCATCCTGGCAGACGCAAACCTGGACAAGGTCCTG | |
| | AGCGCATACAACAAGCACAGAGACAAGCCGATCAGAGAACAGGCAGAAAACATCATCCACCTGTTCACACTGACAAACCTGGGAGC | |
| | ACCGGCAGCATTCAAGTACTTCGACACAACAATCGACAGAAAGAGATACACAAGCACAAAGGAAGTCCTGGACGCAACACTGATCC | |
| | ACCAGAGCATCACAGGACTGTACGAAACAAGAATCGACCTGAGCCAGCTGGGAGGAGACGGAGGAGGAAGCAGAGCAGCAAAGAGA | |
| | AAGTACTTCGCAGTC | |
| mRNA | GGGAAGCUCAGAAUAAACGCUCAACUUUGGCCGGAUCUGCCACCAUGGACAAGAAGUACAGCAUCGGCCUGGACAUCGGCACCAAC | 376 |
| transcript | AGCGUGGGCUGGGCCGUGAUCACCGACGAGUACAAGGUGCCCAGCAAGAAGUUCAAGGUGCUGGGCAACACCGACAGACACAGCAU | |
| with XBG | CAAGAAGAACCUGAUCGGCGCCCUGCUGUUCGACAGCGGCGAGACCGCCGAGGCCACCAGACUGAAGAGAACCGCCAGAAGAAGAU | |
| UTRs and | ACACCAGAAGAAAGAACAGAAUCUGCUACCUGCAGGAGAUCUUCAGCAACGAGAUGGCCAAGGUGGACGACAGCUUCUUCCACAGA | |
| Cas9 ORF | CUGGAGGAGAGCUUCCUGGUGGAGGAGGACAAGAAGCACGAGAGACACCCCAUCUUCGGCAACAUCGUGGACGAGGUGGCCUACCA | |
| with low U 1 | CGAGAAGUACCCCACCAUCUACCACCUGAGAAAGAAGCUGGUGGACAGCACCGACAAGGCCGACCUGAGACUGAUCUACCUGGCCC | |
| codons of | UGGCCCACAUGAUCAAGUUCAGAGGCCACUUCCUGAUCGAGGGCGACCUGAACCCCGACAACAGCGACGUGGACAAGCUGUUCAUC | |
| Table 4 | CAGCUGGUGCAGACCUACAACCAGCUGUUCGAGGAGAACCCCAUCAACGCCAGCGGCGUGGACGCCAAGGCCAUCCUGAGCGCCAG | |
| | ACUGAGCAAGAGCAGAAGACUGGAGAACCUGAUCGCCCAGCUGCCCGGCGAGAAGAAGAACGGCCUGUUCGGCAACCUGAUCGCCC | |
| | UGAGCCUGGGCCUGACCCCCAACUUCAAGAGCAACUUCGACCUGGCCGAGGACGCCAAGCUGCAGCUGAGCAAGGACACCUACGAC | |
| | GACGACCUGGACAACCUGCUGGCCCAGAUCGGCGACCAGUACGCCGACCUGUUCCUGGCCGCCAAGAACCUGAGCGACGCCAUCCU | |
| | GCUGAGCGACAUCCUGAGAGUGAACACCGAGAUCACCAAGGCCCCCCUGAGCGCCAGCAUGAUCAAGAGAUACGACGAGCACCACC | |
| | AGGACCUGACCCUGCUGAAGGCCCUGGUGAGACAGCAGCUGCCCGAGAAGUACAAGGAGAUCUUCUUCGACCAGAGCAAGAACGGC | |
| | UACGCCGGCUACAUCGACGGCGGCGCCAGCCAGGAGGAGUUCUACAAGUUCAUCAAGCCCAUCCUGGAGAAGAUGGACGGCACCGA | |
| | GGAGCUGCUGGUGAAGCUGAACAGAGAGGACCUGCUGAGAAAGCAGAGAACCUUCGACAACGGCAGCAUCCCCCACCAGAUCCACC | |
| | UGGGCGAGCUGCACGCCAUCCUGAGAAGACAGGAGGACUUCUACCCCUUCCUGAAGGACAACAGAGAGAAGAUCGAGAAGAUCCUG | |
| | ACCUUCAGAAUCCCCUACUACGUGGGCCCCCUGGCCAGAGGCAACAGCAGAUUCGCCUGGAUGACCAGAAAGAGCGAGGAGACCAU | |
| | CACCCCCUGGAACUUCGAGGAGGUGGUGGACAAGGGCGCCAGCGCCCAGAGCUUCAUCGAGAGAAUGACCAACUUCGACAAGAACC | |
| | UGCCCAACGAGAAGGUGCUGCCCAAGCACAGCCUGCUGUACGAGUACUUCACCGUGUACAACGAGCUGACCAAGGUGAAGUACGUG | |
| | ACCGAGGGCAUGAGAAAGCCCGCCUUCCUGAGCGGCGAGCAGAAGAAGGCCAUCGUGGACCUGCUGUUCAAGACCAACAGAAAGGU | |
| | GACCGUGAAGCAGCUGAAGGAGGACUACUUCAAGAAGAUCGAGUGCUUCGACAGCGUGGAGAUCAGCGGCGUGGAGGACAGAUUCA | |
| | ACGCCAGCCUGGGCACCUACCACGACCUGCUGAAGAUCAUCAAGGACAAGGACUUCCUGGACAACGAGGAGAACGAGGACAUCCUG | |
| | GAGGACAUCGUGCUGACCCUGACCCUGUUCGAGGACAGAGAGAUGAUCGAGGAGAGACUGAAGACCUACGCCCACCUGUUCGACGA | |
| | CAAGGUGAUGAAGCAGCUGAAGAGAAGAAGAUACACCGGCUGGGGCAGACUGAGCAGAAAGCUGAUCAACGGCAUCAGAGACAAGC | |
| | AGAGCGGCAAGACCAUCCUGGACUUCCUGAAGAGCGACGGCUUCGCCAACAGAAACUUCAUGCAGCUGAUCCACGACGACAGCCUG | |
| | ACCUUCAAGGAGGACAUCCAGAAGGCCCAGGUGAGCGGCCAGGGCGACAGCCUGCACGAGCACAUCGCCAACCUGGCCGGCAGCCC | |
| | CGCCAUCAAGAAGGGCAUCCUGCAGACCGUGAAGGUGGUGGACGAGCUGGUGAAGGUGAUGGGCAGACACAAGCCCGAGAACAUCG | |
| | UGAUCGAGAUGGCCAGAGAGAACCAGACCACCCAGAAGGGCCAGAAGAACAGCAGAGAGAGAAUGAAGAGAAUCGAGGAGGGCAUC | |
| | AAGGAGCUGGGCAGCCAGAUCCUGAAGGAGCACCCCGUGGAGAACACCCAGCUGCAGAACGAGAAGCUGUACCUGUACUACCUGCA | |
| | GAACGGCAGAGACAUGUACGUGGACCAGGAGCUGGACAUCAACAGACUGAGCGACUACGACGUGGACCACAUCGUGCCCCAGAGCU | |
| | UCCUGAAGGACGACAGCAUCGACAACAAGGUGCUGACCAGAAGCGACAAGAACAGAGGCAAGAGCGACAACGUGCCCAGCGAGGAG | |
| | GUGGUGAAGAAGAUGAAGAACUACUGGAGACAGCUGCUGAACGCCAAGCUGAUCACCCAGAGAAAGUUCGACAACCUGACCAAGGC | |
| | CGAGAGAGGCGGCCUGAGCGAGCUGGACAAGGCCGGCUUCAUCAAGAGACAGCUGGUGGAGACCAGACAGAUCACCAAGCACGUGG | |
| | CCCAGAUCCUGGACAGCAGAAUGAACACCAAGUACGACGAGAACGACAAGCUGAUCAGAGAGGUGAAGGUGAUCACCCUGAAGAGC | |
| | AAGCUGGUGAGCGACUUCAGAAAGGACUUCCAGUUCUACAAGGUGAGAGAGAUCAACAACUACCACCACGCCCACGACGCCUACCU | |
| | GAACGCCGUGGUGGGCACCGCCCUGAUCAAGAAGUACCCCAAGCUGGAGAGCGAGUUCGUGUACGGCGACUACAAGGUGUACGACG | |
| | UGAGAAAGAUGAUCGCCAAGAGCGAGCAGGAGAUCGGCAAGGCCACCGCCAAGUACUUCUUCUACAGCAACAUCAUGAACUUCUUC | |
| | AAGACCGAGAUCACCCUGGCCAACGGCGAGAUCAGAAAGAGACCCCUGAUCGAGACCAACGGCGAGACCGGCGAGAUCGUGUGGGA | |
| | CAAGGGCAGAGACUUCGCCACCGUGAGAAAGGUGCUGAGCAUGCCCCAGGUGAACAUCGUGAAGAAGACCGAGGUGCAGACCGGCG | |
| | GCUUCAGCAAGGAGAGCAUCCUGCCCAAGAGAAACAGCGACAAGCUGAUCGCCAGAAAGAAGGACUGGGACCCCAAGAAGUACGGC | |
| | GGCUUCGACAGCCCCACCGUGGCCUACAGCGUGCUGGUGGUGGCCAAGGUGGAGAAGGGCAAGAGCAAGAAGCUGAAGAGCGUGAA | |
| | GGAGCUGCUGGGCAUCACCAUCAUGGAGAGAAGCAGCUUCGAGAAGAACCCCAUCGACUUCCUGGAGGCCAAGGGCUACAAGGAGG | |
| | UGAAGAAGGACCUGAUCAUCAAGCUGCCCAAGUACAGCCUGUUCGAGCUGGAGAACGGCAGAAAGAGAAUGCUGGCCAGCGCCGGC | |
| | GAGCUGCAGAAGGGCAACGAGCUGGCCCUGCCCAGCAAGUACGUGAACUUCCUGUACCUGGCCAGCCACUACGAGAAGCUGAAGGG | |
| | CAGCCCCGAGGACAACGAGCAGAAGCAGCUGUUCGUGGAGCAGCACAAGCACUACCUGGACGAGAUCAUCGAGCAGAUCAGCGAGU | |
| | UCAGCAAGAGAGUGAUCCUGGCCGACGCCAACCUGGACAAGGUGCUGAGCGCCUACAACAAGCACAGAGACAAGCCCAUCAGAGAG | |
| | CAGGCCGAGAACAUCAUCCACCUGUUCACCCUGACCAACCUGGGCGCCCCCGCCGCCUUCAAGUACUUCGACACCACCAUCGACAG | |
| | AAAGAGAUACACCAGCACCAAGGAGGUGCUGGACGCCACCCUGAUCCACCAGAGCAUCACCGGCCUGUACGAGACCAGAAUCGACC | |
| | UGAGCCAGCUGGGCGGCGACGGCGGCGGCAGCCCCAAGAAGAAGAGAAAGGUGUGACUAGCACCAGCCUCAAGAACACCCGAAUGG | |
| | AGUCUCUAAGCUACAUAAUACCAACUUACACUUUACAAAAUGUUGUCCCCCAAAAUGUAGCCAUUCGUAUCUGCUCCUAAUAAAAA | |
| | GAAAGUUUCUUCACAUUCUCUCGAG | |
| mRNA | GGGAAGCUCAGAAUAAACGCUCAACUUUGGCCGGAUCUGCCACCAUGGACAAGAAGUACUCCAUCGGCCUGGACAUCGGCACCAAC | 377 |
| transcript | UCCGUGGGCUGGGCCGUGAUCACCGACGAGUACAAGGUGCCCUCCAAGAAGUUCAAGGUGCUGGGCAACACCGACCGGCACUCCAU | |
| with XBG | CAAGAAGAACCUGAUCGGCGCCCUGCUGUUCGACUCCGGCGAGACCGCCGAGGCCACCCGGCUGAAGCGGACCGCCCGGCGGCGGU | |
| UTRs and | ACACCCGGCGGAAGAACCGGAUCUGCUACCUGCAGGAGAUCUUCUCCAACGAGAUGGCCAAGGUGGACGACUCCUUCUUCCACCGG | |
| Cas9 ORF | CUGGAGGAGUCCUUCCUGGUGGAGGAGGACAAGAAGCACGAGCGGCACCCCAUCUUCGGCAACAUCGUGGACGAGGUGGCCUACCA | |
| with low A | CGAGAAGUACCCCACCAUCUACCACCUGCGGAAGAAGCUGGUGGACUCCACCGACAAGGCCGACCUGCGGCUGAUCUACCUGGCCC | |
| codons of | UGGCCCACAUGAUCAAGUUCCGGGGCCACUUCCUGAUCGAGGGCGACCUGAACCCCGACAACUCCGACGUGGACAAGCUGUUCAUC | |
| Table 4 | CAGCUGGUGCAGACCUACAACCAGCUGUUCGAGGAGAACCCCAUCAACGCCUCCGGCGUGGACGCCAAGGCCAUCCUGUCCGCCCG | |
| | GCUGUCCAAGUCCCGGCGGCUGGAGAACCUGAUCGCCCAGCUGCCCGGCGAGAAGAAGAACGGCCUGUUCGGCAACCUGAUCGCCC | |
| | UGUCCCUGGGCCUGACCCCCAACUUCAAGUCCAACUUCGACCUGGCCGAGGACGCCAAGCUGCAGCUGUCCAAGGACACCUACGAC | |
| | GACGACCUGGACAACCUGCUGGCCCAGAUCGGCGACCAGUACGCCGACCUGUUCCUGGCCGCCAAGAACCUGUCCGACGCCAUCCU | |
| | GCUGUCCGACAUCCUGCGGGUGAACACCGAGAUCACCAAGGCCCCCCUGUCCGCCUCCAUGAUCAAGCGGUACGACGAGCACCACC | |
| | AGGACCUGACCCUGCUGAAGGCCCUGGUGCGGCAGCAGCUGCCCGAGAAGUACAAGGAGAUCUUCUUCGACCAGUCCAAGAACGGC | |
| | UACGCCGGCUACAUCGACGGCGGCGCCUCCCAGGAGGAGUUCUACAAGUUCAUCAAGCCCAUCCUGGAGAAGAUGGACGGCACCGA | |
| | GGAGCUGCUGGUGAAGCUGAACCGGGAGGACCUGCUGCGGAAGCAGCGGACCUUCGACAACGGCUCCAUCCCCCACCAGAUCCACC | |
| | UGGGCGAGCUGCACGCCAUCCUGCGGCGGCAGGAGGACUUCUACCCCUUCCUGAAGGACAACCGGGAGAAGAUCGAGAAGAUCCUG | |
| | ACCUUCCGGAUCCCCUACUACGUGGGCCCCCUGGCCCGGGGCAACUCCCGGUUCGCCUGGAUGACCCGGAAGUCCGAGGAGACCAU | |
| | CACCCCCUGGAACUUCGAGGAGGUGGUGGACAAGGGCGCCUCCGCCCAGUCCUUCAUCGAGCGGAUGACCAACUUCGACAAGAACC | |
| | UGCCCAACGAGAAGGUGCUGCCCAAGCACUCCCUGCUGUACGAGUACUUCACCGUGUACAACGAGCUGACCAAGGUGAAGUACGUG | |
| | ACCGAGGGCAUGCGGAAGCCCGCCUUCCUGUCCGGCGAGCAGAAGAAGGCCAUCGUGGACCUGCUGUUCAAGACCAACCGGAAGGU | |
| | GACCGUGAAGCAGCUGAAGGAGGACUACUUCAAGAAGAUCGAGUGCUUCGACUCCGUGGAGAUCUCCGGCGUGGAGGACCGGUUCA | |
| | ACGCCUCCCUGGGCACCUACCACGACCUGCUGAAGAUCAUCAAGGACAAGGACUUCCUGGACAACGAGGAGAACGAGGACAUCCUG | |
| | GAGGACAUCGUGCUGACCCUGACCCUGUUCGAGGACCGGGAGAUGAUCGAGGAGCGGCUGAAGACCUACGCCCACCUGUUCGACGA | |
| | CAAGGUGAUGAAGCAGCUGAAGCGGCGGCGGUACACCGGCUGGGGCCGGCUGUCCCGGAAGCUGAUCAACGGCAUCCGGGACAAGC | |
| | AGUCCGGCAAGACCAUCCUGGACUUCCUGAAGUCCGACGGCUUCGCCAACCGGAACUUCAUGCAGCUGAUCCACGACGACUCCCUG | |
| | ACCUUCAAGGAGGACAUCCAGAAGGCCCAGGUGUCCGGCCAGGGCGACUCCCUGCACGAGCACAUCGCCAACCUGGCCGGCUCCCC | |
| | CGCCAUCAAGAAGGGCAUCCUGCAGACCGUGAAGGUGGUGGACGAGCUGGUGAAGGUGAUGGGCCGGCACAAGCCCGAGAACAUCG | |
| | UGAUCGAGAUGGCCCGGGAGAACCAGACCACCCAGAAGGGCCAGAAGAACUCCCGGGAGCGGAUGAAGCGGAUCGAGGAGGGCAUC | |
| | AAGGAGCUGGGCUCCCAGAUCCUGAAGGAGCACCCCGUGGAGAACACCCAGCUGCAGAACGAGAAGCUGUACCUGUACUACCUGCA | |
| | GAACGGCCGGGACAUGUACGUGGACCAGGAGCUGGACAUCAACCGGCUGUCCGACUACGACGUGGACCACAUCGUGCCCCAGUCCU | |
| | UCCUGAAGGACGACUCCAUCGACAACAAGGUGCUGACCCGGUCCGACAAGAACCGGGGCAAGUCCGACAACGUGCCCUCCGAGGAG | |
| | GUGGUGAAGAAGAUGAAGAACUACUGGCGGCAGCUGCUGAACGCCAAGCUGAUCACCCAGCGGAAGUUCGACAACCUGACCAAGGC | |
| | CGAGCGGGGCGGCCUGUCCGAGCUGGACAAGGCCGGCUUCAUCAAGCGGCAGCUGGUGGAGACCCGGCAGAUCACCAAGCACGUGG | |
| | CCCAGAUCCUGGACUCCCGGAUGAACACCAAGUACGACGAGAACGACAAGCUGAUCCGGGAGGUGAAGGUGAUCACCCUGAAGUCC | |
| | AAGCUGGUGUCCGACUUCCGGAAGGACUUCCAGUUCUACAAGGUGCGGGAGAUCAACAACUACCACCACGCCCACGACGCCUACCU | |
| | GAACGCCGUGGUGGGCACCGCCCUGAUCAAGAAGUACCCCAAGCUGGAGUCCGAGUUCGUGUACGGCGACUACAAGGUGUACGACG | |
| | UGCGGAAGAUGAUCGCCAAGUCCGAGCAGGAGAUCGGCAAGGCCACCGCCAAGUACUUCUUCUACUCCAACAUCAUGAACUUCUUC | |
| | AAGACCGAGAUCACCCUGGCCAACGGCGAGAUCCGGAAGCGGCCCCUGAUCGAGACCAACGGCGAGACCGGCGAGAUCGUGUGGGA | |
| | CAAGGGCCGGGACUUCGCCACCGUGCGGAAGGUGCUGUCCAUGCCCCAGGUGAACAUCGUGAAGAAGACCGAGGUGCAGACCGGCG | |
| | GCUUCUCCAAGGAGUCCAUCCUGCCCAAGCGGAACUCCGACAAGCUGAUCGCCCGGAAGAAGGACUGGGACCCCAAGAAGUACGGC | |
| | GGCUUCGACUCCCCCACCGUGGCCUACUCCGUGCUGGUGGUGGCCAAGGUGGAGAAGGGCAAGUCCAAGAAGCUGAAGUCCGUGAA | |
| | GGAGCUGCUGGGCAUCACCAUCAUGGAGCGGUCCUCCUUCGAGAAGAACCCCAUCGACUUCCUGGAGGCCAAGGGCUACAAGGAGG | |
| | UGAAGAAGGACCUGAUCAUCAAGCUGCCCAAGUACUCCCUGUUCGAGCUGGAGAACGGCCGGAAGCGGAUGCUGGCCUCCGCCGGC | |
| | GAGCUGCAGAAGGGCAACGAGCUGGCCCUGCCCUCCAAGUACGUGAACUUCCUGUACCUGGCCUCCCACUACGAGAAGCUGAAGGG | |
| | CUCCCCCGAGGACAACGAGCAGAAGCAGCUGUUCGUGGAGCAGCACAAGCACUACCUGGACGAGAUCAUCGAGCAGAUCUCCGAGU | |
| | UCUCCAAGCGGGUGAUCCUGGCCGACGCCAACCUGGACAAGGUGCUGUCCGCCUACAACAAGCACCGGGACAAGCCCAUCCGGGAG | |
| | CAGGCCGAGAACAUCAUCCACCUGUUCACCCUGACCAACCUGGGCGCCCCCGCCGCCUUCAAGUACUUCGACACCACCAUCGACCG | |
| | GAAGCGGUACACCUCCACCAAGGAGGUGCUGGACGCCACCCUGAUCCACCAGUCCAUCACCGGCCUGUACGAGACCCGGAUCGACC | |
| | UGUCCCAGCUGGGCGGCGACGGCGGCGGCUCCCCCAAGAAGAAGCGGAAGGUGUGACUAGCACCAGCCUCAAGAACACCCGAAUGG | |
| | AGUCUCUAAGCUACAUAAUACCAACUUACACUUUACAAAAUGUUGUCCCCCAAAAUGUAGCCAUUCGUAUCUGCUCCUAAUAAAAA | |
| | GAAAGUUUCUUCACAUUCUCUCGAG | |
| mRNA | GGGAAGCUCAGAAUAAACGCUCAACUUUGGCCGGAUCUGCCACCAUGGACAAGAAGUACAGCAUCGGCCUGGACAUCGGCACCAAC | 378 |
| transcript | AGCGUGGGCUGGGCCGUGAUCACCGACGAGUACAAGGUGCCCAGCAAGAAGUUCAAGGUGCUGGGCAACACCGACCGGCACAGCAU | |
| with XBG | CAAGAAGAACCUGAUCGGCGCCCUGCUGUUCGACAGCGGCGAGACCGCCGAGGCCACCCGGCUGAAGCGGACCGCCCGGCGGCGGU | |
| UTRs and | ACACCCGGCGGAAGAACCGGAUCUGCUACCUGCAGGAGAUCUUCAGCAACGAGAUGGCCAAGGUGGACGACAGCUUCUUCCACCGG | |
| Cas9 ORF | CUGGAGGAGAGCUUCCUGGUGGAGGAGGACAAGAAGCACGAGCGGCACCCCAUCUUCGGCAACAUCGUGGACGAGGUGGCCUACCA | |
| with low U/A | CGAGAAGUACCCCACCAUCUACCACCUGCGGAAGAAGCUGGUGGACAGCACCGACAAGGCCGACCUGCGGCUGAUCUACCUGGCCC | |
| codons of | UGGCCCACAUGAUCAAGUUCCGGGGCCACUUCCUGAUCGAGGGCGACCUGAACCCCGACAACAGCGACGUGGACAAGCUGUUCAUC | |
| Table 4 | CAGCUGGUGCAGACCUACAACCAGCUGUUCGAGGAGAACCCCAUCAACGCCAGCGGCGUGGACGCCAAGGCCAUCCUGAGCGCCCG | |
| | GCUGAGCAAGAGCCGGCGGCUGGAGAACCUGAUCGCCCAGCUGCCCGGCGAGAAGAAGAACGGCCUGUUCGGCAACCUGAUCGCCC | |
| | UGAGCCUGGGCCUGACCCCCAACUUCAAGAGCAACUUCGACCUGGCCGAGGACGCCAAGCUGCAGCUGAGCAAGGACACCUACGAC | |
| | GACGACCUGGACAACCUGCUGGCCCAGAUCGGCGACCAGUACGCCGACCUGUUCCUGGCCGCCAAGAACCUGAGCGACGCCAUCCU | |
| | GCUGAGCGACAUCCUGCGGGUGAACACCGAGAUCACCAAGGCCCCCCUGAGCGCCAGCAUGAUCAAGCGGUACGACGAGCACCACC | |
| | AGGACCUGACCCUGCUGAAGGCCCUGGUGCGGCAGCAGCUGCCCGAGAAGUACAAGGAGAUCUUCUUCGACCAGAGCAAGAACGGC | |
| | UACGCCGGCUACAUCGACGGCGGCGCCAGCCAGGAGGAGUUCUACAAGUUCAUCAAGCCCAUCCUGGAGAAGAUGGACGGCACCGA | |
| | GGAGCUGCUGGUGAAGCUGAACCGGGAGGACCUGCUGCGGAAGCAGCGGACCUUCGACAACGGCAGCAUCCCCCACCAGAUCCACC | |
| | UGGGCGAGCUGCACGCCAUCCUGCGGCGGCAGGAGGACUUCUACCCCUUCCUGAAGGACAACCGGGAGAAGAUCGAGAAGAUCCUG | |
| | ACCUUCCGGAUCCCCUACUACGUGGGCCCCCUGGCCCGGGGCAACAGCCGGUUCGCCUGGAUGACCCGGAAGAGCGAGGAGACCAU | |
| | CACCCCCUGGAACUUCGAGGAGGUGGUGGACAAGGGCGCCAGCGCCCAGAGCUUCAUCGAGCGGAUGACCAACUUCGACAAGAACC | |
| | UGCCCAACGAGAAGGUGCUGCCCAAGCACAGCCUGCUGUACGAGUACUUCACCGUGUACAACGAGCUGACCAAGGUGAAGUACGUG | |
| | ACCGAGGGCAUGCGGAAGCCCGCCUUCCUGAGCGGCGAGCAGAAGAAGGCCAUCGUGGACCUGCUGUUCAAGACCAACCGGAAGGU | |
| | GACCGUGAAGCAGCUGAAGGAGGACUACUUCAAGAAGAUCGAGUGCUUCGACAGCGUGGAGAUCAGCGGCGUGGAGGACCGGUUCA | |
| | ACGCCAGCCUGGGCACCUACCACGACCUGCUGAAGAUCAUCAAGGACAAGGACUUCCUGGACAACGAGGAGAACGAGGACAUCCUG | |
| | GAGGACAUCGUGCUGACCCUGACCCUGUUCGAGGACCGGGAGAUGAUCGAGGAGCGGCUGAAGACCUACGCCCACCUGUUCGACGA | |
| | CAAGGUGAUGAAGCAGCUGAAGCGGCGGCGGUACACCGGCUGGGGCCGGCUGAGCCGGAAGCUGAUCAACGGCAUCCGGGACAAGC | |
| | AGAGCGGCAAGACCAUCCUGGACUUCCUGAAGAGCGACGGCUUCGCCAACCGGAACUUCAUGCAGCUGAUCCACGACGACAGCCUG | |
| | ACCUUCAAGGAGGACAUCCAGAAGGCCCAGGUGAGCGGCCAGGGCGACAGCCUGCACGAGCACAUCGCCAACCUGGCCGGCAGCCC | |
| | CGCCAUCAAGAAGGGCAUCCUGCAGACCGUGAAGGUGGUGGACGAGCUGGUGAAGGUGAUGGGCCGGCACAAGCCCGAGAACAUCG | |
| | UGAUCGAGAUGGCCCGGGAGAACCAGACCACCCAGAAGGGCCAGAAGAACAGCCGGGAGCGGAUGAAGCGGAUCGAGGAGGGCAUC | |
| | AAGGAGCUGGGCAGCCAGAUCCUGAAGGAGCACCCCGUGGAGAACACCCAGCUGCAGAACGAGAAGCUGUACCUGUACUACCUGCA | |
| | GAACGGCCGGGACAUGUACGUGGACCAGGAGCUGGACAUCAACCGGCUGAGCGACUACGACGUGGACCACAUCGUGCCCCAGAGCU | |
| | UCCUGAAGGACGACAGCAUCGACAACAAGGUGCUGACCCGGAGCGACAAGAACCGGGGCAAGAGCGACAACGUGCCCAGCGAGGAG | |
| | GUGGUGAAGAAGAUGAAGAACUACUGGCGGCAGCUGCUGAACGCCAAGCUGAUCACCCAGCGGAAGUUCGACAACCUGACCAAGGC | |
| | CGAGCGGGGCGGCCUGAGCGAGCUGGACAAGGCCGGCUUCAUCAAGCGGCAGCUGGUGGAGACCCGGCAGAUCACCAAGCACGUGG | |
| | CCCAGAUCCUGGACAGCCGGAUGAACACCAAGUACGACGAGAACGACAAGCUGAUCCGGGAGGUGAAGGUGAUCACCCUGAAGAGC | |
| | AAGCUGGUGAGCGACUUCCGGAAGGACUUCCAGUUCUACAAGGUGCGGGAGAUCAACAACUACCACCACGCCCACGACGCCUACCU | |
| | GAACGCCGUGGUGGGCACCGCCCUGAUCAAGAAGUACCCCAAGCUGGAGAGCGAGUUCGUGUACGGCGACUACAAGGUGUACGACG | |
| | UGCGGAAGAUGAUCGCCAAGAGCGAGCAGGAGAUCGGCAAGGCCACCGCCAAGUACUUCUUCUACAGCAACAUCAUGAACUUCUUC | |
| | AAGACCGAGAUCACCCUGGCCAACGGCGAGAUCCGGAAGCGGCCCCUGAUCGAGACCAACGGCGAGACCGGCGAGAUCGUGUGGGA | |
| | CAAGGGCCGGGACUUCGCCACCGUGCGGAAGGUGCUGAGCAUGCCCCAGGUGAACAUCGUGAAGAAGACCGAGGUGCAGACCGGCG | |
| | GCUUCAGCAAGGAGAGCAUCCUGCCCAAGCGGAACAGCGACAAGCUGAUCGCCCGGAAGAAGGACUGGGACCCCAAGAAGUACGGC | |
| | GGCUUCGACAGCCCCACCGUGGCCUACAGCGUGCUGGUGGUGGCCAAGGUGGAGAAGGGCAAGAGCAAGAAGCUGAAGAGCGUGAA | |
| | GGAGCUGCUGGGCAUCACCAUCAUGGAGCGGAGCAGCUUCGAGAAGAACCCCAUCGACUUCCUGGAGGCCAAGGGCUACAAGGAGG | |
| | UGAAGAAGGACCUGAUCAUCAAGCUGCCCAAGUACAGCCUGUUCGAGCUGGAGAACGGCCGGAAGCGGAUGCUGGCCAGCGCCGGC | |
| | GAGCUGCAGAAGGGCAACGAGCUGGCCCUGCCCAGCAAGUACGUGAACUUCCUGUACCUGGCCAGCCACUACGAGAAGCUGAAGGG | |
| | CAGCCCCGAGGACAACGAGCAGAAGCAGCUGUUCGUGGAGCAGCACAAGCACUACCUGGACGAGAUCAUCGAGCAGAUCAGCGAGU | |
| | UCAGCAAGCGGGUGAUCCUGGCCGACGCCAACCUGGACAAGGUGCUGAGCGCCUACAACAAGCACCGGGACAAGCCCAUCCGGGAG | |
| | CAGGCCGAGAACAUCAUCCACCUGUUCACCCUGACCAACCUGGGCGCCCCCGCCGCCUUCAAGUACUUCGACACCACCAUCGACCG | |
| | GAAGCGGUACACCAGCACCAAGGAGGUGCUGGACGCCACCCUGAUCCACCAGAGCAUCACCGGCCUGUACGAGACCCGGAUCGACC | |
| | UGAGCCAGCUGGGCGGCGACGGCGGCGGCAGCCCCAAGAAGAAGCGGAAGGUGUGACUAGCACCAGCCUCAAGAACACCCGAAUGG | |
| | AGUCUCUAAGCUACAUAAUACCAACUUACACUUUACAAAAUGUUGUCCCCCAAAAUGUAGCCAUUCGUAUCUGCUCCUAAUAAAAA | |
| | GAAAGUUUCUUCACAUUCUCUCGAG | |
| mRNA | GGGTCCCGCAGTCGGCGTCCAGCGGCTCTGCTTGTTCGTGTGTGTGTCGTTGCAGGCCTTATTCGGATCCGCCACCATGGACAAGA | 379 |
| transcript | AGTACAGCATCGGACTGGACATCGGAACAAACAGCGTCGGATGGGCAGTCATCACAGACGAATACAAGGTCCCGAGCAAGAAGTTC | |
| with ORF | AAGGTCCTGGGAAACACAGACAGACACAGCATCAAGAAGAACCTGATCGGAGCACTGCTGTTCGACAGCGGAGAAACAGCAGAAGC | |
| encoding | AACAAGACTGAAGAGAACAGCAAGAAGAAGATACACAAGAAGAAAGAACAGAATCTGCTACCTGCAGGAAATCTTCAGCAACGAAA | |
| Cas9 with | TGGCAAAGGTCGACGACAGCTTCTTCCACAGACTGGAAGAAAGCTTCCTGGTCGAAGAAGACAAGAAGCACGAAAGACACCCGATC | |
| HiBiT tag, | TTCGGAAACATCGTCGACGAAGTCGCATACCACGAAAAGTACCCGACAATCTACCACCTGAGAAAGAAGCTGGTCGACAGCACAGA | |
| HSD 5′ UTR | CAAGGCAGACCTGAGACTGATCTACCTGGCACTGGCACACATGATCAAGTTCAGAGGACACTTCCTGATCGAAGGAGACCTGAACC | |
| and human | CGGACAACAGCGACGTCGACAAGCTGTTCATCCAGCTGGTCCAGACATACAACCAGCTGTTCGAAGAAAACCCGATCAACGCAAGC | |
| ALB 3′ UTR | GGAGTCGACGCAAAGGCAATCCTGAGCGCAAGACTGAGCAAGAGCAGAAGACTGGAAAACCTGATCGCACAGCTGCCGGGAGAAAA | |
| | GAAGAACGGACTGTTCGGAAACCTGATCGCACTGAGCCTGGGACTGACACCGAACTTCAAGAGCAACTTCGACCTGGCAGAAGACG | |
| | CAAAGCTGCAGCTGAGCAAGGACACATACGACGACGACCTGGACAACCTGCTGGCACAGATCGGAGACCAGTACGCAGACCTGTTC | |
| | CTGGCAGCAAAGAACCTGAGCGACGCAATCCTGCTGAGCGACATCCTGAGAGTCAACACAGAAATCACAAAGGCACCGCTGAGCGC | |
| | AAGCATGATCAAGAGATACGACGAACACCACCAGGACCTGACACTGCTGAAGGCACTGGTCAGACAGCAGCTGCCGGAAAAGTACA | |
| | AGGAAATCTTCTTCGACCAGAGCAAGAACGGATACGCAGGATACATCGACGGAGGAGCAAGCCAGGAAGAATTCTACAAGTTCATC | |
| | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCA | |
| | GCGAAAGCGCAACACCGGAAAGCGTCAGCGGATGGAGACTGTTCAAGAAGATCAGCTAGCTAGCCATCACATTTAAAAGCATCTCA | |
| | GCCTACCATGAGAATAAGAGAAAGAAAATGAAGATCAATAGCTTATTCATCTCTTTTTCTTTTTCGTTGGTGTAAAGCCAACACCC | |
| | TGTCTAAAAAACATAAATTTCTTTAATCATTTTGCCTCTTTTCTCTGTGCTTCAATTAATAAAAAATGGAAAGAACCTCGAGAAAA | |
| mRNA | GGGCAGATCGCCTGGAGACGCCATCCACGCTGTTTTGACCTCCATCGCCACCATGGACAAGAAGTACAGCATCGGACTGGACATCG | 380 |
| transcript | GAACAAACAGCGTCGGATGGGCAGTCATCACAGACGAATACAAGGTCCCGAGCAAGAAGTTCAAGGTCCTGGGAAACACAGACAGA | |
| with ORF | CACAGCATCAAGAAGAACCTGATCGGAGCACTGCTGTTCGACAGCGGAGAAACAGCAGAAGCAACAAGACTGAAGAGAACAGCAAG | |
| encoding | AAGAAGATACACAAGAAGAAAGAACAGAATCTGCTACCTGCAGGAAATCTTCAGCAACGAAATGGCAAAGGTCGACGACAGCTTCT | |
| Cas9 with | TCCACAGACTGGAAGAAAGCTTCCTGGTCGAAGAAGACAAGAAGCACGAAAGACACCCGATCTTCGGAAACATCGTCGACGAAGTC | |
| HiBiT tag, | GCATACCACGAAAAGTACCCGACAATCTACCACCTGAGAAAGAAGCTGGTCGACAGCACAGACAAGGCAGACCTGAGACTGATCTA | |
| CMV-1 5′ UTR | CCTGGCACTGGCACACATGATCAAGTTCAGAGGACACTTCCTGATCGAAGGAGACCTGAACCCGGACAACAGCGACGTCGACAAGC | |
| and human | TGTTCATCCAGCTGGTCCAGACATACAACCAGCTGTTCGAAGAAAACCCGATCAACGCAAGCGGAGTCGACGCAAAGGCAATCCTG | |
| ALB 3′ UTR | AGCGCAAGACTGAGCAAGAGCAGAAGACTGGAAAACCTGATCGCACAGCTGCCGGGAGAAAAGAAGAACGGACTGTTCGGAAACCT | |
| | GATCGCACTGAGCCTGGGACTGACACCGAACTTCAAGAGCAACTTCGACCTGGCAGAAGACGCAAAGCTGCAGCTGAGCAAGGACA | |
| | CATACGACGACGACCTGGACAACCTGCTGGCACAGATCGGAGACCAGTACGCAGACCTGTTCCTGGCAGCAAAGAACCTGAGCGAC | |
| | GCAATCCTGCTGAGCGACATCCTGAGAGTCAACACAGAAATCACAAAGGCACCGCTGAGCGCAAGCATGATCAAGAGATACGACGA | |
| | ACACCACCAGGACCTGACACTGCTGAAGGCACTGGTCAGACAGCAGCTGCCGGAAAAGTACAAGGAAATCTTCTTCGACCAGAGCA | |
| | AGAACGGATACGCAGGATACATCGACGGAGGAGCAAGCCAGGAAGAATTCTACAAGTTCATCAAGCCGATCCTGGAAAAGATGGAC | |
| | GGAACAGAAGAACTGCTGGTCAAGCTGAACAGAGAAGACCTGCTGAGAAAGCAGAGAACATTCGACAACGGAAGCATCCCGCACCA | |
| | GATCCACCTGGGAGAACTGCACGCAATCCTGAGAAGACAGGAAGACTTCTACCCGTTCCTGAAGGACAACAGAGAAAAGATCGAAA | |
| | AGATCCTGACATTCAGAATCCCGTACTACGTCGGACCGCTGGCAAGAGGAAACAGCAGATTCGCATGGATGACAAGAAAGAGCGAA | |
| | GAAACAATCACACCGTGGAACTTCGAAGAAGTCGTCGACAAGGGAGCAAGCGCACAGAGCTTCATCGAAAGAATGACAAACTTCGA | |
| | CAAGAACCTGCCGAACGAAAAGGTCCTGCCGAAGCACAGCCTGCTGTACGAATACTTCACAGTCTACAACGAACTGACAAAGGTCA | |
| | AGTACGTCACAGAAGGAATGAGAAAGCCGGCATTCCTGAGCGGAGAACAGAAGAAGGCAATCGTCGACCTGCTGTTCAAGACAAAC | |
| | AGAAAGGTCACAGTCAAGCAGCTGAAGGAAGACTACTTCAAGAAGATCGAATGCTTCGACAGCGTCGAAATCAGCGGAGTCGAAGA | |
| | CAGATTCAACGCAAGCCTGGGAACATACCACGACCTGCTGAAGATCATCAAGGACAAGGACTTCCTGGACAACGAAGAAAACGAAG | |
| | ACATCCTGGAAGACATCGTCCTGACACTGACACTGTTCGAAGACAGAGAAATGATCGAAGAAAGACTGAAGACATACGCACACCTG | |
| | TTCGACGACAAGGTCATGAAGCAGCTGAAGAGAAGAAGATACACAGGATGGGGAAGACTGAGCAGAAAGCTGATCAACGGAATCAG | |
| | AGACAAGCAGAGCGGAAAGACAATCCTGGACTTCCTGAAGAGCGACGGATTCGCAAACAGAAACTTCATGCAGCTGATCCACGACG | |
| | ACAGCCTGACATTCAAGGAAGACATCCAGAAGGCACAGGTCAGCGGACAGGGAGACAGCCTGCACGAACACATCGCAAACCTGGCA | |
| | GGAAGCCCGGCAATCAAGAAGGGAATCCTGCAGACAGTCAAGGTCGTCGACGAACTGGTCAAGGTCATGGGAAGACACAAGCCGGA | |
| | AAACATCGTCATCGAAATGGCAAGAGAAAACCAGACAACACAGAAGGGACAGAAGAACAGCAGAGAAAGAATGAAGAGAATCGAAG | |
| | AAGGAATCAAGGAACTGGGAAGCCAGATCCTGAAGGAACACCCGGTCGAAAACACACAGCTGCAGAACGAAAAGCTGTACCTGTAC | |
| | TACCTGCAGAACGGAAGAGACATGTACGTCGACCAGGAACTGGACATCAACAGACTGAGCGACTACGACGTCGACCACATCGTCCC | |
| | GCAGAGCTTCCTGAAGGACGACAGCATCGACAACAAGGTCCTGACAAGAAGCGACAAGAACAGAGGAAAGAGCGACAACGTCCCGA | |
| | GCGAAGAAGTCGTCAAGAAGATGAAGAACTACTGGAGACAGCTGCTGAACGCAAAGCTGATCACACAGAGAAAGTTCGACAACCTG | |
| | ACAAAGGCAGAGAGAGGAGGACTGAGCGAACTGGACAAGGCAGGATTCATCAAGAGACAGCTGGTCGAAACAAGACAGATCACAAA | |
| | GCACGTCGCACAGATCCTGGACAGCAGAATGAACACAAAGTACGACGAAAACGACAAGCTGATCAGAGAAGTCAAGGTCATCACAC | |
| | TGAAGAGCAAGCTGGTCAGCGACTTCAGAAAGGACTTCCAGTTCTACAAGGTCAGAGAAATCAACAACTACCACCACGCACACGAC | |
| | GCATACCTGAACGCAGTCGTCGGAACAGCACTGATCAAGAAGTACCCGAAGCTGGAAAGCGAATTCGTCTACGGAGACTACAAGGT | |
| | CTACGACGTCAGAAAGATGATCGCAAAGAGCGAACAGGAAATCGGAAAGGCAACAGCAAAGTACTTCTTCTACAGCAACATCATGA | |
| | ACTTCTTCAAGACAGAAATCACACTGGCAAACGGAGAAATCAGAAAGAGACCGCTGATCGAAACAAACGGAGAAACAGGAGAAATC | |
| | GTCTGGGACAAGGGAAGAGACTTCGCAACAGTCAGAAAGGTCCTGAGCATGCCGCAGGTCAACATCGTCAAGAAGACAGAAGTCCA | |
| | GACAGGAGGATTCAGCAAGGAAAGCATCCTGCCGAAGAGAAACAGCGACAAGCTGATCGCAAGAAAGAAGGACTGGGACCCGAAGA | |
| | AGTACGGAGGATTCGACAGCCCGACAGTCGCATACAGCGTCCTGGTCGTCGCAAAGGTCGAAAAGGGAAAGAGCAAGAAGCTGAAG | |
| | AGCGTCAAGGAACTGCTGGGAATCACAATCATGGAAAGAAGCAGCTTCGAAAAGAACCCGATCGACTTCCTGGAAGCAAAGGGATA | |
| | CAAGGAAGTCAAGAAGGACCTGATCATCAAGCTGCCGAAGTACAGCCTGTTCGAACTGGAAAACGGAAGAAAGAGAATGCTGGCAA | |
| | GCGCAGGAGAACTGCAGAAGGGAAACGAACTGGCACTGCCGAGCAAGTACGTCAACTTCCTGTACCTGGCAAGCCACTACGAAAAG | |
| | CTGAAGGGAAGCCCGGAAGACAACGAACAGAAGCAGCTGTTCGTCGAACAGCACAAGCACTACCTGGACGAAATCATCGAACAGAT | |
| | CAGCGAATTCAGCAAGAGAGTCATCCTGGCAGACGCAAACCTGGACAAGGTCCTGAGCGCATACAACAAGCACAGAGACAAGCCGA | |
| | TCAGAGAACAGGCAGAAAACATCATCCACCTGTTCACACTGACAAACCTGGGAGCACCGGCAGCATTCAAGTACTTCGACACAACA | |
| | ATCGACAGAAAGAGATACACAAGCACAAAGGAAGTCCTGGACGCAACACTGATCCACCAGAGCATCACAGGACTGTACGAAACAAG | |
| | AATCGACCTGAGCCAGCTGGGAGGAGACGGAGGAGGAAGCCCGAAGAAGAAGAGAAAGGTCAGCGAAAGCGCAACACCGGAAAGCG | |
| | TCAGCGGATGGAGACTGTTCAAGAAGATCAGCTAGCTAGCCATCACATTTAAAAGCATCTCAGCCTACCATGAGAATAAGAGAAAG | |
| | AAAATGAAGATCAATAGCTTATTCATCTCTTTTTCTTTTTCGTTGGTGTAAAGCCAACACCCTGTCTAAAAAACATAAATTTCTTT | |
| | AATCATTTTGCCTCTTTTCTCTGTGCTTCAATTAATAAAAAATGGAAAGAACCTCGAG | |
| mRNA | GGGAGAAGACACCGGGACCGATCCAGCCTCCGCGGCCGGGAACGGCGCCACCATGGACAAGAAGTACAGCATCGGACTGGACATCG | 381 |
| transcript | GAACAAACAGCGTCGGATGGGCAGTCATCACAGACGAATACAAGGTCCCGAGCAAGAAGTTCAAGGTCCTGGGAAACACAGACAGA | |
| with ORF | CACAGCATCAAGAAGAACCTGATCGGAGCACTGCTGTTCGACAGCGGAGAAACAGCAGAAGCAACAAGACTGAAGAGAACAGCAAG | |
| encoding | AAGAAGATACACAAGAAGAAAGAACAGAATCTGCTACCTGCAGGAAATCTTCAGCAACGAAATGGCAAAGGTCGACGACAGCTTCT | |
| Cas9 with | TCCACAGACTGGAAGAAAGCTTCCTGGTCGAAGAAGACAAGAAGCACGAAAGACACCCGATCTTCGGAAACATCGTCGACGAAGTC | |
| HiBiT tag, | GCATACCACGAAAAGTACCCGACAATCTACCACCTGAGAAAGAAGCTGGTCGACAGCACAGACAAGGCAGACCTGAGACTGATCTA | |
| CNV-2 5′ UTR | CCTGGCACTGGCACACATGATCAAGTTCAGAGGACACTTCCTGATCGAAGGAGACCTGAACCCGGACAACAGCGACGTCGACAAGC | |
| and human | TGTTCATCCAGCTGGTCCAGACATACAACCAGCTGTTCGAAGAAAACCCGATCAACGCAAGCGGAGTCGACGCAAAGGCAATCCTG | |
| ALB 3′ UTR | AGCGCAAGACTGAGCAAGAGCAGAAGACTGGAAAACCTGATCGCACAGCTGCCGGGAGAAAAGAAGAACGGACTGTTCGGAAACCT | |
| | GATCGCACTGAGCCTGGGACTGACACCGAACTTCAAGAGCAACTTCGACCTGGCAGAAGACGCAAAGCTGCAGCTGAGCAAGGACA | |
| | CATACGACGACGACCTGGACAACCTGCTGGCACAGATCGGAGACCAGTACGCAGACCTGTTCCTGGCAGCAAAGAACCTGAGCGAC | |
| | GCAATCCTGCTGAGCGACATCCTGAGAGTCAACACAGAAATCACAAAGGCACCGCTGAGCGCAAGCATGATCAAGAGATACGACGA | |
| | ACACCACCAGGACCTGACACTGCTGAAGGCACTGGTCAGACAGCAGCTGCCGGAAAAGTACAAGGAAATCTTCTTCGACCAGAGCA | |
| | AGAACGGATACGCAGGATACATCGACGGAGGAGCAAGCCAGGAAGAATTCTACAAGTTCATCAAGCCGATCCTGGAAAAGATGGAC | |
| | GGAACAGAAGAACTGCTGGTCAAGCTGAACAGAGAAGACCTGCTGAGAAAGCAGAGAACATTCGACAACGGAAGCATCCCGCACCA | |
| | GATCCACCTGGGAGAACTGCACGCAATCCTGAGAAGACAGGAAGACTTCTACCCGTTCCTGAAGGACAACAGAGAAAAGATCGAAA | |
| | AGATCCTGACATTCAGAATCCCGTACTACGTCGGACCGCTGGCAAGAGGAAACAGCAGATTCGCATGGATGACAAGAAAGAGCGAA | |
| | GAAACAATCACACCGTGGAACTTCGAAGAAGTCGTCGACAAGGGAGCAAGCGCACAGAGCTTCATCGAAAGAATGACAAACTTCGA | |
| | CAAGAACCTGCCGAACGAAAAGGTCCTGCCGAAGCACAGCCTGCTGTACGAATACTTCACAGTCTACAACGAACTGACAAAGGTCA | |
| | AGTACGTCACAGAAGGAATGAGAAAGCCGGCATTCCTGAGCGGAGAACAGAAGAAGGCAATCGTCGACCTGCTGTTCAAGACAAAC | |
| | AGAAAGGTCACAGTCAAGCAGCTGAAGGAAGACTACTTCAAGAAGATCGAATGCTTCGACAGCGTCGAAATCAGCGGAGTCGAAGA | |
| | CAGATTCAACGCAAGCCTGGGAACATACCACGACCTGCTGAAGATCATCAAGGACAAGGACTTCCTGGACAACGAAGAAAACGAAG | |
| | ACATCCTGGAAGACATCGTCCTGACACTGACACTGTTCGAAGACAGAGAAATGATCGAAGAAAGACTGAAGACATACGCACACCTG | |
| | TTCGACGACAAGGTCATGAAGCAGCTGAAGAGAAGAAGATACACAGGATGGGGAAGACTGAGCAGAAAGCTGATCAACGGAATCAG | |
| | AGACAAGCAGAGCGGAAAGACAATCCTGGACTTCCTGAAGAGCGACGGATTCGCAAACAGAAACTTCATGCAGCTGATCCACGACG | |
| | ACAGCCTGACATTCAAGGAAGACATCCAGAAGGCACAGGTCAGCGGACAGGGAGACAGCCTGCACGAACACATCGCAAACCTGGCA | |
| | GGAAGCCCGGCAATCAAGAAGGGAATCCTGCAGACAGTCAAGGTCGTCGACGAACTGGTCAAGGTCATGGGAAGACACAAGCCGGA | |
| | AAACATCGTCATCGAAATGGCAAGAGAAAACCAGACAACACAGAAGGGACAGAAGAACAGCAGAGAAAGAATGAAGAGAATCGAAG | |
| | AAGGAATCAAGGAACTGGGAAGCCAGATCCTGAAGGAACACCCGGTCGAAAACACACAGCTGCAGAACGAAAAGCTGTACCTGTAC | |
| | TACCTGCAGAACGGAAGAGACATGTACGTCGACCAGGAACTGGACATCAACAGACTGAGCGACTACGACGTCGACCACATCGTCCC | |
| | GCAGAGCTTCCTGAAGGACGACAGCATCGACAACAAGGTCCTGACAAGAAGCGACAAGAACAGAGGAAAGAGCGACAACGTCCCGA | |
| | GCGAAGAAGTCGTCAAGAAGATGAAGAACTACTGGAGACAGCTGCTGAACGCAAAGCTGATCACACAGAGAAAGTTCGACAACCTG | |
| | ACAAAGGCAGAGAGAGGAGGACTGAGCGAACTGGACAAGGCAGGATTCATCAAGAGACAGCTGGTCGAAAGAAGACAGATCACAAA | |
| | GCACGTCGCACAGATCCTGGACAGCAGAATGAACACAAAGTACGACGAAAACGACAAGCTGATCAGAGAAGTCAAGGTCATCACAC | |
| | TGAAGAGCAAGCTGGTCAGCGACTTCAGAAAGGACTTCCAGTTCTACAAGGTCAGAGAAATCAACAACTACCACCACGCACACGAC | |
| | GCATACCTGAACGCAGTCGTCGGAACAGCACTGATCAAGAAGTACCCGAAGCTGGAAAGCGAATTCGTCTACGGAGACTACAAGGT | |
| | CTACGACGTCAGAAAGATGATCGCAAAGAGCGAACAGGAAATCGGAAAGGCAACAGCAAAGTACTTCTTCTACAGCAACATCATGA | |
| | ACTTCTTCAAGACAGAAATCACACTGGCAAACGGAGAAATCAGAAAGAGACCGCTGATCGAAACAAACGGAGAAACAGGAGAAATC | |
| | GTCTGGGACAAGGGAAGAGACTTCGCAACAGTCAGAAAGGTCCTGAGCATGCCGCAGGTCAACATCGTCAAGAAGACAGAAGTCCA | |
| | GACAGGAGGATTCAGCAAGGAAAGCATCCTGCCGAAGAGAAACAGCGACAAGCTGATCGCAAGAAAGAAGGACTGGGACCCGAAGA | |
| | AGTACGGAGGATTCGACAGCCCGACAGTCGCATACAGCGTCCTGGTCGTCGCAAAGGTCGAAAAGGGAAAGAGCAAGAAGCTGAAG | |
| | AGCGTCAAGGAACTGCTGGGAATCACAATCATGGAAAGAAGCAGCTTCGAAAAGAACCCGATCGACTTCCTGGAAGCAAAGGGATA | |
| | CAAGGAAGTCAAGAAGGACCTGATCATCAAGCTGCCGAAGTACAGCCTGTTCGAACTGGAAAACGGAAGAAAGAGAATGCTGGCAA | |
| | GCGCAGGAGAACTGCAGAAGGGAAACGAACTGGCACTGCCGAGCAAGTACGTCAACTTCCTGTACCTGGCAAGCCACTACGAAAAG | |
| | CTGAAGGGAAGCCCGGAAGACAACGAACAGAAGCAGCTGTTCGTCGAACAGCACAAGCACTACCTGGACGAAATCATCGAACAGAT | |
| | CAGCGAATTCAGCAAGAGAGTCATCCTGGCAGACGCAAACCTGGACAAGGTCCTGAGCGCATACAACAAGCACAGAGACAAGCCGA | |
| | TCAGAGAACAGGCAGAAAACATCATCCACCTGTTCACACTGACAAACCTGGGAGCACCGGCAGCATTCAAGTACTTCGACACAACA | |
| | ATCGACAGAAAGAGATACACAAGCACAAAGGAAGTCCTGGACGCAACACTGATCCACCAGAGCATCACAGGACTGTACGAAACAAG | |
| | AATCGACCTGAGCCAGCTGGGAGGAGACGGAGGAGGAAGCCCGAAGAAGAAGAGAAAGGTCAGCGAAAGCGCAACACCGGAAAGCG | |
| | TCAGCGGATGGAGACTGTTCAAGAAGATCAGCTAGCTAGCCATCACATTTAAAAGCATCTCAGCCTACCATGAGAATAAGAGAAAG | |
| | AAAATGAAGATCAATAGCTTATTCATCTCTTTTTCTTTTTCGTTGGTGTAAAGCCAACACCCTGTCTAAAAAACATAAATTTCTTT | |
| | AATCATTTTGCCTCTTTTCTCTGTGCTTCAATTAATAAAAAATGGAAAGAACCTCGAG | |
| mRNA | GGGTGCATTGGAACGCGGATTCCCCGTGCCAAGAGTGACTCACCGCGCCACCATGGACAAGAAGTACAGCATCGGACTGGACATCG | 382 |
| transcript | GAACAAACAGCGTCGGATGGGCAGTCATCACAGACGAATACAAGGTCCCGAGCAAGAAGTTCAAGGTCCTGGGAAACACAGACAGA | |
| with ORF | CACAGCATCAAGAAGAACCTGATCGGAGCACTGCTGTTCGACAGCGGAGAAACAGCAGAAGCAACAAGACTGAAGAGAACAGCAAG | |
| encoding | AAGAAGATACACAAGAAGAAAGAACAGAATCTGCTACCTGCAGGAAATCTTCAGCAACGAAATGGCAAAGGTCGACGACAGCTTCT | |
| Cas9 with | TCCACAGACTGGAAGAAAGCTTCCTGGTCGAAGAAGACAAGAAGCACGAAAGACACCCGATCTTCGGAAACATCGTCGACGAAGTC | |
| HiBiT tag, | GCATACCACGAAAAGTACCCGACAATCTACCACCTGAGAAAGAAGCTGGTCGACAGCACAGACAAGGCAGACCTGAGACTGATCTA | |
| CNV-3 5′ UTR | CCTGGCACTGGCACACATGATCAAGTTCAGAGGACACTTCCTGATCGAAGGAGACCTGAACCCGGACAACAGCGACGTCGACAAGC | |
| and human | TGTTCATCCAGCTGGTCCAGACATACAACCAGCTGTTCGAAGAAAACCCGATCAACGCAAGCGGAGTCGACGCAAAGGCAATCCTG | |
| ALB 3′ UTR | AGCGCAAGACTGAGCAAGAGCAGAAGACTGGAAAACCTGATCGCACAGCTGCCGGGAGAAAAGAAGAACGGACTGTTCGGAAACCT | |
| | GATCGCACTGAGCCTGGGACTGACACCGAACTTCAAGAGCAACTTCGACCTGGCAGAAGACGCAAAGCTGCAGCTGAGCAAGGACA | |
| | CATACGACGACGACCTGGACAACCTGCTGGCACAGATCGGAGACCAGTACGCAGACCTGTTCCTGGCAGCAAAGAACCTGAGCGAC | |
| | GCAATCCTGCTGAGCGACATCCTGAGAGTCAACACAGAAATCACAAAGGCACCGCTGAGCGCAAGCATGATCAAGAGATACGACGA | |
| | ACACCACCAGGACCTGACACTGCTGAAGGCACTGGTCAGACAGCAGCTGCCGGAAAAGTACAAGGAAATCTTCTTCGACCAGAGCA | |
| | AGAACGGATACGCAGGATACATCGACGGAGGAGCAAGCCAGGAAGAATTCTACAAGTTCATCAAGCCGATCCTGGAAAAGATGGAC | |
| | GGAACAGAAGAACTGCTGGTCAAGCTGAACAGAGAAGACCTGCTGAGAAAGCAGAGAACATTCGACAACGGAAGCATCCCGCACCA | |
| | GATCCACCTGGGAGAACTGCACGCAATCCTGAGAAGACAGGAAGACTTCTACCCGTTCCTGAAGGACAACAGAGAAAAGATCGAAA | |
| | AGATCCTGACATTCAGAATCCCGTACTACGTCGGACCGCTGGCAAGAGGAAACAGCAGATTCGCATGGATGACAAGAAAGAGCGAA | |
| | GAAACAATCACACCGTGGAACTTCGAAGAAGTCGTCGACAAGGGAGCAAGCGCACAGAGCTTCATCGAAAGAATGACAAACTTCGA | |
| | CAAGAACCTGCCGAACGAAAAGGTCCTGCCGAAGCACAGCCTGCTGTACGAATACTTCACAGTCTACAACGAACTGACAAAGGTCA | |
| | AGTACGTCACAGAAGGAATGAGAAAGCCGGCATTCCTGAGCGGAGAACAGAAGAAGGCAATCGTCGACCTGCTGTTCAAGACAAAC | |
| | AGAAAGGTCACAGTCAAGCAGCTGAAGGAAGACTACTTCAAGAAGATCGAATGCTTCGACAGCGTCGAAATCAGCGGAGTCGAAGA | |
| | CAGATTCAACGCAAGCCTGGGAACATACCACGACCTGCTGAAGATCATCAAGGACAAGGACTTCCTGGACAACGAAGAAAACGAAG | |
| | ACATCCTGGAAGACATCGTCCTGACACTGACACTGTTCGAAGACAGAGAAATGATCGAAGAAAGACTGAAGACATACGCACACCTG | |
| | TTCGACGACAAGGTCATGAAGCAGCTGAAGAGAAGAAGATACACAGGATGGGGAAGACTGAGCAGAAAGCTGATCAACGGAATCAG | |
| | AGACAAGCAGAGCGGAAAGACAATCCTGGACTTCCTGAAGAGCGACGGATTCGCAAACAGAAACTTCATGCAGCTGATCCACGACG | |
| | ACAGCCTGACATTCAAGGAAGACATCCAGAAGGCACAGGTCAGCGGACAGGGAGACAGCCTGCACGAACACATCGCAAACCTGGCA | |
| | GGAAGCCCGGCAATCAAGAAGGGAATCCTGCAGACAGTCAAGGTCGTCGACGAACTGGTCAAGGTCATGGGAAGACACAAGCCGGA | |
| | AAACATCGTCATCGAAATGGCAAGAGAAAACCAGACAACACAGAAGGGACAGAAGAACAGCAGAGAAAGAATGAAGAGAATCGAAG | |
| | AAGGAATCAAGGAACTGGGAAGCCAGATCCTGAAGGAACACCCGGTCGAAAACACACAGCTGCAGAACGAAAAGCTGTACCTGTAC | |
| | TACCTGCAGAACGGAAGAGACATGTACGTCGACCAGGAACTGGACATCAACAGACTGAGCGACTACGACGTCGACCACATCGTCCC | |
| | GCAGAGCTTCCTGAAGGACGACAGCATCGACAACAAGGTCCTGACAAGAAGCGACAAGAACAGAGGAAAGAGCGACAACGTCCCGA | |
| | GCGAAGAAGTCGTCAAGAAGATGAAGAACTACTGGAGACAGCTGCTGAACGCAAAGCTGATCACACAGAGAAAGTTCGACAACCTG | |
| | ACAAAGGCAGAGAGAGGAGGACTGAGCGAACTGGACAAGGCAGGATTCATCAAGAGACAGCTGGTCGAAACAAGACAGATCACAAA | |
| | GCACGTCGCACAGATCCTGGACAGCAGAATGAACACAAAGTACGACGAAAACGACAAGCTGATCAGAGAAGTCAAGGTCATCACAC | |
| | TGAAGAGCAAGCTGGTCAGCGACTTCAGAAAGGACTTCCAGTTCTACAAGGTCAGAGAAATCAACAACTACCACCACGCACACGAC | |
| | GCATACCTGAACGCAGTCGTCGGAACAGCACTGATCAAGAAGTACCCGAAGCTGGAAAGCGAATTCGTCTACGGAGACTACAAGGT | |
| | CTACGACGTCAGAAAGATGATCGCAAAGAGCGAACAGGAAATCGGAAAGGCAACAGCAAAGTACTTCTTCTACAGCAACATCATGA | |
| | ACTTCTTCAAGACAGAAATCACACTGGCAAACGGAGAAATCAGAAAGAGACCGCTGATCGAAACAAACGGAGAAACAGGAGAAATC | |
| | GTCTGGGACAAGGGAAGAGACTTCGCAACAGTCAGAAAGGTCCTGAGCATGCCGCAGGTCAACATCGTCAAGAAGACAGAAGTCCA | |
| | GACAGGAGGATTCAGCAAGGAAAGCATCCTGCCGAAGAGAAACAGCGACAAGCTGATCGCAAGAAAGAAGGACTGGGACCCGAAGA | |
| | AGTACGGAGGATTCGACAGCCCGACAGTCGCATACAGCGTCCTGGTCGTCGCAAAGGTCGAAAAGGGAAAGAGCAAGAAGCTGAAG | |
| | AGCGTCAAGGAACTGCTGGGAATCACAATCATGGAAAGAAGCAGCTTCGAAAAGAACCCGATCGACTTCCTGGAAGCAAAGGGATA | |
| | CAAGGAAGTCAAGAAGGACCTGATCATCAAGCTGCCGAAGTACAGCCTGTTCGAACTGGAAAACGGAAGAAAGAGAATGCTGGCAA | |
| | GCGCAGGAGAACTGCAGAAGGGAAACGAACTGGCACTGCCGAGCAAGTACGTCAACTTCCTGTACCTGGCAAGCCACTACGAAAAG | |
| | CTGAAGGGAAGCCCGGAAGACAACGAACAGAAGCAGCTGTTCGTCGAACAGCACAAGCACTACCTGGACGAAATCATCGAACAGAT | |
| | CAGCGAATTCAGCAAGAGAGTCATCCTGGCAGACGCAAACCTGGACAAGGTCCTGAGCGCATACAACAAGCACAGAGACAAGCCGA | |
| | TCAGAGAACAGGCAGAAAACATCATCCACCTGTTCACACTGACAAACCTGGGAGCACCGGCAGCATTCAAGTACTTCGACACAACA | |
| | ATCGACAGAAAGAGATACACAAGCACAAAGGAAGTCCTGGACGCAACACTGATCCACCAGAGCATCACAGGACTGTACGAAACAAG | |
| | AATCGACCTGAGCCAGCTGGGAGGAGACGGAGGAGGAAGCCCGAAGAAGAAGAGAAAGGTCAGCGAAAGCGCAACACCGGAAAGCG | |
| | TCAGCGGATGGAGACTGTTCAAGAAGATCAGCTAGCTAGCCATCACATTTAAAAGCATCTCAGCCTACCATGAGAATAAGAGAAAG | |
| | AAAATGAAGATCAATAGCTTATTCATCTCTTTTTCTTTTTCGTTGGTGTAAAGCCAACACCCTGTCTAAAAAACATAAATTTCTTT | |
| | AATCATTTTGCCTCTTTTCTCTGTGCTTCAATTAATAAAAAATGGAAAGAACCTCGAG | |
| mRNA | GGGCATAAACCCTGGCGCGCTCGCGGCCCGGCACTCTTCTGGTCCCCACAGACTCAGAGAGAACCCACCCGCCACCATGGACAAGA | 383 |
| transcript | AGTACAGCATCGGACTGGACATCGGAACAAACAGCGTCGGATGGGCAGTCATCACAGACGAATACAAGGTCCCGAGCAAGAAGTTC | |
| with ORF | AAGGTCCTGGGAAACACAGACAGACACAGCATCAAGAAGAACCTGATCGGAGCACTGCTGTTCGACAGCGGAGAAACAGCAGAAGC | |
| encoding | AACAAGACTGAAGAGAACAGCAAGAAGAAGATACACAAGAAGAAAGAACAGAATCTGCTACCTGCAGGAAATCTTCAGCAACGAAA | |
| Cas9 with | TGGCAAAGGTCGACGACAGCTTCTTCCACAGACTGGAAGAAAGCTTCCTGGTCGAAGAAGACAAGAAGCACGAAAGACACCCGATC | |
| HiBiT tag, | TTCGGAAACATCGTCGACGAAGTCGCATACCACGAAAAGTACCCGACAATCTACCACCTGAGAAAGAAGCTGGTCGACAGCACAGA | |
| HBA 5′ UTR | CAAGGCAGACCTGAGACTGATCTACCTGGCACTGGCACACATGATCAAGTTCAGAGGACACTTCCTGATCGAAGGAGACCTGAACC | |
| and human | CGGACAACAGCGACGTCGACAAGCTGTTCATCCAGCTGGTCCAGACATACAACCAGCTGTTCGAAGAAAACCCGATCAACGCAAGC | |
| ALB 3′ UTR | GGAGTCGACGCAAAGGCAATCCTGAGCGCAAGACTGAGCAAGAGCAGAAGACTGGAAAACCTGATCGCACAGCTGCCGGGAGAAAA | |
| | GAAGAACGGACTGTTCGGAAACCTGATCGCACTGAGCCTGGGACTGACACCGAACTTCAAGAGCAACTTCGACCTGGCAGAAGACG | |
| | CAAAGCTGCAGCTGAGCAAGGACACATACGACGACGACCTGGACAACCTGCTGGCACAGATCGGAGACCAGTACGCAGACCTGTTC | |
| | CTGGCAGCAAAGAACCTGAGCGACGCAATCCTGCTGAGCGACATCCTGAGAGTCAACACAGAAATCACAAAGGCACCGCTGAGCGC | |
| | AAGCATGATCAAGAGATACGACGAACACCACCAGGACCTGACACTGCTGAAGGCACTGGTCAGACAGCAGCTGCCGGAAAAGTACA | |
| | AGGAAATCTTCTTCGACCAGAGCAAGAACGGATACGCAGGATACATCGACGGAGGAGCAAGCCAGGAAGAATTCTACAAGTTCATC | |
| | AAGCCGATCCTGGAAAAGATGGACGGAACAGAAGAACTGCTGGTCAAGCTGAACAGAGAAGACCTGCTGAGAAAGCAGAGAACATT | |
| | CGACAACGGAAGCATCCCGCACCAGATCCACCTGGGAGAACTGCACGCAATCCTGAGAAGACAGGAAGACTTCTACCCGTTCCTGA | |
| | AGGACAACAGAGAAAAGATCGAAAAGATCCTGACATTCAGAATCCCGTACTACGTCGGACCGCTGGCAAGAGGAAACAGCAGATTC | |
| | GCATGGATGACAAGAAAGAGCGAAGAAACAATCACACCGTGGAACTTCGAAGAAGTCGTCGACAAGGGAGCAAGCGCACAGAGCTT | |
| | CATCGAAAGAATGACAAACTTCGACAAGAACCTGCCGAACGAAAAGGTCCTGCCGAAGCACAGCCTGCTGTACGAATACTTCACAG | |
| | TCTACAACGAACTGACAAAGGTCAAGTACGTCACAGAAGGAATGAGAAAGCCGGCATTCCTGAGCGGAGAACAGAAGAAGGCAATC | |
| | GTCGACCTGCTGTTCAAGACAAACAGAAAGGTCACAGTCAAGCAGCTGAAGGAAGACTACTTCAAGAAGATCGAATGCTTCGACAG | |
| | CGTCGAAATCAGCGGAGTCGAAGACAGATTCAACGCAAGCCTGGGAACATACCACGACCTGCTGAAGATCATCAAGGACAAGGACT | |
| | TCCTGGACAACGAAGAAAACGAAGACATCCTGGAAGACATCGTCCTGACACTGACACTGTTCGAAGACAGAGAAATGATCGAAGAA | |
| | AGACTGAAGACATACGCACACCTGTTCGACGACAAGGTCATGAAGCAGCTGAAGAGAAGAAGATACACAGGATGGGGAAGACTGAG | |
| | CAGAAAGCTGATCAACGGAATCAGAGACAAGCAGAGCGGAAAGACAATCCTGGACTTCCTGAAGAGCGACGGATTCGCAAACAGAA | |
| | ACTTCATGCAGCTGATCCACGACGACAGCCTGACATTCAAGGAAGACATCCAGAAGGCACAGGTCAGCGGACAGGGAGACAGCCTG | |
| | CACGAACACATCGCAAACCTGGCAGGAAGCCCGGCAATCAAGAAGGGAATCCTGCAGACAGTCAAGGTCGTCGACGAACTGGTCAA | |
| | GGTCATGGGAAGACACAAGCCGGAAAACATCGTCATCGAAATGGCAAGAGAAAACCAGACAACACAGAAGGGACAGAAGAACAGCA | |
| | GAGAAAGAATGAAGAGAATCGAAGAAGGAATCAAGGAACTGGGAAGCCAGATCCTGAAGGAACACCCGGTCGAAAACACACAGCTG | |
| | CAGAACGAAAAGCTGTACCTGTACTACCTGCAGAACGGAAGAGACATGTACGTCGACCAGGAACTGGACATCAACAGACTGAGCGA | |
| | CTACGACGTCGACCACATCGTCCCGCAGAGCTTCCTGAAGGACGACAGCATCGACAACAAGGTCCTGACAAGAAGCGACAAGAACA | |
| | GAGGAAAGAGCGACAACGTCCCGAGCGAAGAAGTCGTCAAGAAGATGAAGAACTACTGGAGACAGCTGCTGAACGCAAAGCTGATC | |
| | ACACAGAGAAAGTTCGACAACCTGACAAAGGCAGAGAGAGGAGGACTGAGCGAACTGGACAAGGCAGGATTCATCAAGAGACAGCT | |
| | GGTCGAAACAAGACAGATCACAAAGCACGTCGCACAGATCCTGGACAGCAGAATGAACACAAAGTACGACGAAAACGACAAGCTGA | |
| | TCAGAGAAGTCAAGGTCATCACACTGAAGAGCAAGCTGGTCAGCGACTTCAGAAAGGACTTCCAGTTCTACAAGGTCAGAGAAATC | |
| | AACAACTACCACCACGCACACGACGCATACCTGAACGCAGTCGTCGGAACAGCACTGATCAAGAAGTACCCGAAGCTGGAAAGCGA | |
| | ATTCGTCTACGGAGACTACAAGGTCTACGACGTCAGAAAGATGATCGCAAAGAGCGAACAGGAAATCGGAAAGGCAACAGCAAAGT | |
| | ACTTCTTCTACAGCAACATCATGAACTTCTTCAAGACAGAAATCACACTGGCAAACGGAGAAATCAGAAAGAGACCGCTGATCGAA | |
| | ACAAACGGAGAAACAGGAGAAATCGTCTGGGACAAGGGAAGAGACTTCGCAACAGTCAGAAAGGTCCTGAGCATGCCGCAGGTCAA | |
| | CATCGTCAAGAAGACAGAAGTCCAGACAGGAGGATTCAGCAAGGAAAGCATCCTGCCGAAGAGAAACAGCGACAAGCTGATCGCAA | |
| | GAAAGAAGGACTGGGACCCGAAGAAGTACGGAGGATTCGACAGCCCGACAGTCGCATACAGCGTCCTGGTCGTCGCAAAGGTCGAA | |
| | AAGGGAAAGAGCAAGAAGCTGAAGAGCGTCAAGGAACTGCTGGGAATCACAATCATGGAAAGAAGCAGCTTCGAAAAGAACCCGAT | |
| | CGACTTCCTGGAAGCAAAGGGATACAAGGAAGTCAAGAAGGACCTGATCATCAAGCTGCCGAAGTACAGCCTGTTCGAACTGGAAA | |
| | ACGGAAGAAAGAGAATGCTGGCAAGCGCAGGAGAACTGCAGAAGGGAAACGAACTGGCACTGCCGAGCAAGTACGTCAACTTCCTG | |
| | TACCTGGCAAGCCACTACGAAAAGCTGAAGGGAAGCCCGGAAGACAACGAACAGAAGCAGCTGTTCGTCGAACAGCACAAGCACTA | |
| | CCTGGACGAAATCATCGAACAGATCAGCGAATTCAGCAAGAGAGTCATCCTGGCAGACGCAAACCTGGACAAGGTCCTGAGCGCAT | |
| | ACAACAAGCACAGAGACAAGCCGATCAGAGAACAGGCAGAAAACATCATCCACCTGTTCACACTGACAAACCTGGGAGCACCGGCA | |
| | GCATTCAAGTACTTCGACACAACAATCGACAGAAAGAGATACACAAGCACAAAGGAAGTCCTGGACGCAACACTGATCCACCAGAG | |
| | CATCACAGGACTGTACGAAACAAGAATCGACCTGAGCCAGCTGGGAGGAGACGGAGGAGGAAGCCCGAAGAAGAAGAGAAAGGTCA | |
| | GCGAAAGCGCAACACCGGAAAGCGTCAGCGGATGGAGACTGTTCAAGAAGATCAGCTAGCTAGCCATCACATTTAAAAGCATCTCA | |
| | GCCTACCATGAGAATAAGAGAAAGAAAATGAAGATCAATAGCTTATTCATCTCTTTTTCTTTTTCGTTGGTGTAAAGCCAACACCC | |
| | TGTCTAAAAAACATAAATTTCTTTAATCATTTTGCCTCTTTTCTCTGTGCTTCAATTAATAAAAAATGGAAAGAACCTCGAGAAAA | |
| mRNA | GGGACATTTGCTTCTGACACAACTGTGTTCACTAGCAACCTCAAACAGACACCGGATCTCGCCACCATGGACAAGAAGTACAGCAT | 384 |
| transcript | CGGACTGGACATCGGAACAAACAGCGTCGGATGGGCAGTCATCACAGACGAATACAAGGTCCCGAGCAAGAAGTTCAAGGTCCTGG | |
| with ORF | GAAACACAGACAGACACAGCATCAAGAAGAACCTGATCGGAGCACTGCTGTTCGACAGCGGAGAAACAGCAGAAGCAACAAGACTG | |
| encoding | AAGAGAACAGCAAGAAGAAGATACACAAGAAGAAAGAACAGAATCTGCTACCTGCAGGAAATCTTCAGCAACGAAATGGCAAAGGT | |
| Cas9 with | CGACGACAGCTTCTTCCACAGACTGGAAGAAAGCTTCCTGGTCGAAGAAGACAAGAAGCACGAAAGACACCCGATCTTCGGAAACA | |
| HiBiT tag, | TCGTCGACGAAGTCGCATACCACGAAAAGTACCCGACAATCTACCACCTGAGAAAGAAGCTGGTCGACAGCACAGACAAGGCAGAC | |
| HBB 5′ UTR | CTGAGACTGATCTACCTGGCACTGGCACACATGATCAAGTTCAGAGGACACTTCCTGATCGAAGGAGACCTGAACCCGGACAACAG | |
| and human | CGACGTCGACAAGCTGTTCATCCAGCTGGTCCAGACATACAACCAGCTGTTCGAAGAAAACCCGATCAACGCAAGCGGAGTCGACG | |
| ALB 3′ UTR | CAAAGGCAATCCTGAGCGCAAGACTGAGCAAGAGCAGAAGACTGGAAAACCTGATCGCACAGCTGCCGGGAGAAAAGAAGAACGGA | |
| | CTGTTCGGAAACCTGATCGCACTGAGCCTGGGACTGACACCGAACTTCAAGAGCAACTTCGACCTGGCAGAAGACGCAAAGCTGCA | |
| | GCTGAGCAAGGACACATACGACGACGACCTGGACAACCTGCTGGCACAGATCGGAGACCAGTACGCAGACCTGTTCCTGGCAGCAA | |
| | AGAACCTGAGCGACGCAATCCTGCTGAGCGACATCCTGAGAGTCAACACAGAAATCACAAAGGCACCGCTGAGCGCAAGCATGATC | |
| | AAGAGATACGACGAACACCACCAGGACCTGACACTGCTGAAGGCACTGGTCAGACAGCAGCTGCCGGAAAAGTACAAGGAAATCTT | |
| | CTTCGACCAGAGCAAGAACGGATACGCAGGATACATCGACGGAGGAGCAAGCCAGGAAGAATTCTACAAGTTCATCAAGCCGATCC | |
| | TGGAAAAGATGGACGGAAGAGAAGAACTGCTGGTCAAGCTGAACAGAGAAGACCTGCTGAGAAAGCAGAGAACATTCGACAACGGA | |
| | AGCATCCCGCACCAGATCCACCTGGGAGAACTGCACGCAATCCTGAGAAGACAGGAAGACTTCTACCCGTTCCTGAAGGACAACAG | |
| | AGAAAAGATCGAAAAGATCCTGACATTCAGAATCCCGTACTACGTCGGACCGCTGGCAAGAGGAAACAGCAGATTCGCATGGATGA | |
| | CAAGAAAGAGCGAAGAAACAATCACACCGTGGAACTTCGAAGAAGTCGTCGACAAGGGAGCAAGCGCACAGAGCTTCATCGAAAGA | |
| | ATGACAAACTTCGACAAGAACCTGCCGAACGAAAAGGTCCTGCCGAAGCACAGCCTGCTGTACGAATACTTCACAGTCTACAACGA | |
| | ACTGACAAAGGTCAAGTACGTCACAGAAGGAATGAGAAAGCCGGCATTCCTGAGCGGAGAACAGAAGAAGGCAATCGTCGACCTGC | |
| | TGTTCAAGACAAACAGAAAGGTCACAGTCAAGCAGCTGAAGGAAGACTACTTCAAGAAGATCGAATGCTTCGACAGCGTCGAAATC | |
| | AGCGGAGTCGAAGACAGATTCAACGCAAGCCTGGGAACATACCACGACCTGCTGAAGATCATCAAGGACAAGGACTTCCTGGACAA | |
| | CGAAGAAAACGAAGACATCCTGGAAGACATCGTCCTGACACTGACACTGTTCGAAGACAGAGAAATGATCGAAGAAAGACTGAAGA | |
| | CATACGCACACCTGTTCGACGACAAGGTCATGAAGCAGCTGAAGAGAAGAAGATACACAGGATGGGGAAGACTGAGCAGAAAGCTG | |
| | ATCAACGGAATCAGAGACAAGCAGAGCGGAAAGACAATCCTGGACTTCCTGAAGAGCGACGGATTCGCAAACAGAAACTTCATGCA | |
| | GCTGATCCACGACGACAGCCTGACATTCAAGGAAGACATCCAGAAGGCACAGGTCAGCGGACAGGGAGACAGCCTGCACGAACACA | |
| | TCGCAAACCTGGCAGGAAGCCCGGCAATCAAGAAGGGAATCCTGCAGACAGTCAAGGTCGTCGACGAACTGGTCAAGGTCATGGGA | |
| | AGACACAAGCCGGAAAACATCGTCATCGAAATGGCAAGAGAAAACCAGACAACACAGAAGGGACAGAAGAACAGCAGAGAAAGAAT | |
| | GAAGAGAATCGAAGAAGGAATCAAGGAACTGGGAAGCCAGATCCTGAAGGAACACCCGGTCGAAAACACACAGCTGCAGAACGAAA | |
| | AGCTGTACCTGTACTACCTGCAGAACGGAAGAGACATGTACGTCGACCAGGAACTGGACATCAACAGACTGAGCGACTACGACGTC | |
| | GACCACATCGTCCCGCAGAGCTTCCTGAAGGACGACAGCATCGACAACAAGGTCCTGACAAGAAGCGACAAGAACAGAGGAAAGAG | |
| | CGACAACGTCCCGAGCGAAGAAGTCGTCAAGAAGATGAAGAACTACTGGAGACAGCTGCTGAACGCAAAGCTGATCACACAGAGAA | |
| | AGTTCGACAACCTGACAAAGGCAGAGAGAGGAGGACTGAGCGAACTGGACAAGGCAGGATTCATCAAGAGACAGCTGGTCGAAACA | |
| | AGACAGATCACAAAGCACGTCGCACAGATCCTGGACAGCAGAATGAACACAAAGTACGACGAAAACGACAAGCTGATCAGAGAAGT | |
| | CAAGGTCATCACACTGAAGAGCAAGCTGGTCAGCGACTTCAGAAAGGACTTCCAGTTCTACAAGGTCAGAGAAATCAACAACTACC | |
| | ACCACGCACACGACGCATACCTGAACGCAGTCGTCGGAACAGCACTGATCAAGAAGTACCCGAAGCTGGAAAGCGAATTCGTCTAC | |
| | GGAGACTACAAGGTCTACGACGTCAGAAAGATGATCGCAAAGAGCGAACAGGAAATCGGAAAGGCAACAGCAAAGTACTTCTTCTA | |
| | CAGCAACATCATGAACTTCTTCAAGACAGAAATCACACTGGCAAACGGAGAAATCAGAAAGAGACCGCTGATCGAAACAAACGGAG | |
| | AAACAGGAGAAATCGTCTGGGACAAGGGAAGAGACTTCGCAACAGTCAGAAAGGTCCTGAGCATGCCGCAGGTCAACATCGTCAAG | |
| | AAGACAGAAGTCCAGACAGGAGGATTCAGCAAGGAAAGCATCCTGCCGAAGAGAAACAGCGACAAGCTGATCGCAAGAAAGAAGGA | |
| | CTGGGACCCGAAGAAGTACGGAGGATTCGACAGCCCGACAGTCGCATACAGCGTCCTGGTCGTCGCAAAGGTCGAAAAGGGAAAGA | |
| | GCAAGAAGCTGAAGAGCGTCAAGGAACTGCTGGGAATCACAATCATGGAAAGAAGCAGCTTCGAAAAGAACCCGATCGACTTCCTG | |
| | GAAGCAAAGGGATACAAGGAAGTCAAGAAGGACCTGATCATCAAGCTGCCGAAGTACAGCCTGTTCGAACTGGAAAACGGAAGAAA | |
| | GAGAATGCTGGCAAGCGCAGGAGAACTGCAGAAGGGAAACGAACTGGCACTGCCGAGCAAGTACGTCAACTTCCTGTACCTGGCAA | |
| | GCCACTACGAAAAGCTGAAGGGAAGCCCGGAAGACAACGAACAGAAGCAGCTGTTCGTCGAACAGCACAAGCACTACCTGGACGAA | |
| | ATCATCGAACAGATCAGCGAATTCAGCAAGAGAGTCATCCTGGCAGACGCAAACCTGGACAAGGTCCTGAGCGCATACAACAAGCA | |
| | CAGAGACAAGCCGATCAGAGAACAGGCAGAAAACATCATCCACCTGTTCACACTGACAAACCTGGGAGCACCGGCAGCATTCAAGT | |
| | ACTTCGACACAACAATCGACAGAAAGAGATACACAAGCACAAAGGAAGTCCTGGACGCAACACTGATCCACCAGAGCATCACAGGA | |
| | CTGTACGAAACAAGAATCGACCTGAGCCAGCTGGGAGGAGACGGAGGAGGAAGCCCGAAGAAGAAGAGAAAGGTCAGCGAAAGCGC | |
| | AACACCGGAAAGCGTCAGCGGATGGAGACTGTTCAAGAAGATCAGCTAGCTAGCCATCACATTTAAAAGCATCTCAGCCTACCATG | |
| | AGAATAAGAGAAAGAAAATGAAGATCAATAGCTTATTCATCTCTTTTTCTTTTTCGTTGGTGTAAAGCCAACACCCTGTCTAAAAA | |
| | ACATAAATTTCTTTAATCATTTTGCCTCTTTTCTCTGTGCTTCAATTAATAAAAAATGGAAAGAACCTCGAGAAAAAAAAAAAAAA | |
| | AAAAAAAAAAAAAAAAAAAAAAAAAAAAAAAAAAAAAAAAAAAAAAAAAAAAAAAAAAAAAAAAAA | |
| mRNA | GGGAAGCTCAGAATAAACGCTCAACTTTGGCCGGATCTCGCCACCATGGACAAGAAGTACAGCATCGGACTGGACATCGGAACAAA | 385 |
| transcript | CAGCGTCGGATGGGCAGTCATCACAGACGAATACAAGGTCCCGAGCAAGAAGTTCAAGGTCCTGGGAAACACAGACAGACACAGCA | |
| with ORF | TCAAGAAGAACCTGATCGGAGCACTGCTGTTCGACAGCGGAGAAACAGCAGAAGCAACAAGACTGAAGAGAACAGCAAGAAGAAGA | |
| encoding | TACACAAGAAGAAAGAACAGAATCTGCTACCTGCAGGAAATCTTCAGCAACGAAATGGCAAAGGTCGACGACAGCTTCTTCCACAG | |
| Cas9 with | ACTGGAAGAAAGCTTCCTGGTCGAAGAAGACAAGAAGCACGAAAGACACCCGATCTTCGGAAACATCGTCGACGAAGTCGCATACC | |
| HiBiT tag, | ACGAAAAGTACCCGACAATCTACCACCTGAGAAAGAAGCTGGTCGACAGCACAGACAAGGCAGACCTGAGACTGATCTACCTGGCA | |
| XBG 5′ UTR | CTGGCACACATGATCAAGTTCAGAGGACACTTCCTGATCGAAGGAGACCTGAACCCGGACAACAGCGACGTCGACAAGCTGTTCAT | |
| and human | CCAGCTGGTCCAGACATACAACCAGCTGTTCGAAGAAAACCCGATCAACGCAAGCGGAGTCGACGCAAAGGCAATCCTGAGCGCAA | |
| ALB 3′ UTR | GACTGAGCAAGAGCAGAAGACTGGAAAACCTGATCGCACAGCTGCCGGGAGAAAAGAAGAACGGACTGTTCGGAAACCTGATCGCA | |
| | CTGAGCCTGGGACTGACACCGAACTTCAAGAGCAACTTCGACCTGGCAGAAGACGCAAAGCTGCAGCTGAGCAAGGACACATACGA | |
| | CGACGACCTGGACAACCTGCTGGCACAGATCGGAGACCAGTACGCAGACCTGTTCCTGGCAGCAAAGAACCTGAGCGACGCAATCC | |
| | TGCTGAGCGACATCCTGAGAGTCAACACAGAAATCACAAAGGCACCGCTGAGCGCAAGCATGATCAAGAGATACGACGAACACCAC | |
| | CAGGACCTGACACTGCTGAAGGCACTGGTCAGACAGCAGCTGCCGGAAAAGTACAAGGAAATCTTCTTCGACCAGAGCAAGAACGG | |
| | ATACGCAGGATACATCGACGGAGGAGCAAGCCAGGAAGAATTCTACAAGTTCATCAAGCCGATCCTGGAAAAGATGGACGGAACAG | |
| | AAGAACTGCTGGTCAAGCTGAACAGAGAAGACCTGCTGAGAAAGCAGAGAACATTCGACAACGGAAGCATCCCGCACCAGATCCAC | |
| | CTGGGAGAACTGCACGCAATCCTGAGAAGACAGGAAGACTTCTACCCGTTCCTGAAGGACAACAGAGAAAAGATCGAAAAGATCCT | |
| | GACATTCAGAATCCCGTACTACGTCGGACCGCTGGCAAGAGGAAACAGCAGATTCGCATGGATGACAAGAAAGAGCGAAGAAACAA | |
| | TCACACCGTGGAACTTCGAAGAAGTCGTCGACAAGGGAGCAAGCGCACAGAGCTTCATCGAAAGAATGACAAACTTCGACAAGAAC | |
| | CTGCCGAACGAAAAGGTCCTGCCGAAGCACAGCCTGCTGTACGAATACTTCACAGTCTACAACGAACTGACAAAGGTCAAGTACGT | |
| | CACAGAAGGAATGAGAAAGCCGGCATTCCTGAGCGGAGAACAGAAGAAGGCAATCGTCGACCTGCTGTTCAAGACAAACAGAAAGG | |
| | TCACAGTCAAGCAGCTGAAGGAAGACTACTTCAAGAAGATCGAATGCTTCGACAGCGTCGAAATCAGCGGAGTCGAAGACAGATTC | |
| | AACGCAAGCCTGGGAACATACCACGACCTGCTGAAGATCATCAAGGACAAGGACTTCCTGGACAACGAAGAAAACGAAGACATCCT | |
| | GGAAGACATCGTCCTGACACTGACACTGTTCGAAGACAGAGAAATGATCGAAGAAAGACTGAAGACATACGCACACCTGTTCGACG | |
| | ACAAGGTCATGAAGCAGCTGAAGAGAAGAAGATACACAGGATGGGGAAGACTGAGCAGAAAGCTGATCAACGGAATCAGAGACAAG | |
| | CAGAGCGGAAAGACAATCCTGGACTTCCTGAAGAGCGACGGATTCGCAAACAGAAACTTCATGCAGCTGATCCACGACGACAGCCT | |
| | GACATTCAAGGAAGACATCCAGAAGGCACAGGTCAGCGGACAGGGAGACAGCCTGCACGAACACATCGCAAACCTGGCAGGAAGCC | |
| | CGGCAATCAAGAAGGGAATCCTGCAGACAGTCAAGGTCGTCGACGAACTGGTCAAGGTCATGGGAAGACACAAGCCGGAAAACATC | |
| | GTCATCGAAATGGCAAGAGAAAACCAGACAACACAGAAGGGACAGAAGAACAGCAGAGAAAGAATGAAGAGAATCGAAGAAGGAAT | |
| | CAAGGAACTGGGAAGCCAGATCCTGAAGGAACACCCGGTCGAAAAGACACAGCTGCAGAACGAAAAGCTGTACCTGTACTACCTGC | |
| | AGAACGGAAGAGACATGTACGTCGACCAGGAACTGGACATCAACAGACTGAGCGACTACGACGTCGACCACATCGTCCCGCAGAGC | |
| | TTCCTGAAGGACGACAGCATCGACAACAAGGTCCTGACAAGAAGCGACAAGAACAGAGGAAAGAGCGACAACGTCCCGAGCGAAGA | |
| | AGTCGTCAAGAAGATGAAGAACTACTGGAGACAGCTGCTGAACGCAAAGCTGATCACACAGAGAAAGTTCGACAACCTGACAAAGG | |
| | CAGAGAGAGGAGGACTGAGCGAACTGGACAAGGCAGGATTCATCAAGAGACAGCTGGTCGAAACAAGACAGATCACAAAGCACGTC | |
| | GCACAGATCCTGGACAGCAGAATGAACACAAAGTACGACGAAAACGACAAGCTGATCAGAGAAGTCAAGGTCATCACACTGAAGAG | |
| | CAAGCTGGTCAGCGACTTCAGAAAGGACTTCCAGTTCTACAAGGTCAGAGAAATCAACAACTACCACCACGCACACGACGCATACC | |
| | TGAACGCAGTCGTCGGAACAGCACTGATCAAGAAGTACCCGAAGCTGGAAAGCGAATTCGTCTACGGAGACTACAAGGTCTACGAC | |
| | GTCAGAAAGATGATCGCAAAGAGCGAACAGGAAATCGGAAAGGCAACAGCAAAGTACTTCTTCTACAGCAACATCATGAACTTCTT | |
| | CAAGACAGAAATCACACTGGCAAACGGAGAAATCAGAAAGAGACCGCTGATCGAAACAAACGGAGAAACAGGAGAAATCGTCTGGG | |
| | ACAAGGGAAGAGACTTCGCAACAGTCAGAAAGGTCCTGAGCATGCCGCAGGTCAACATCGTCAAGAAGACAGAAGTCCAGACAGGA | |
| | GGATTCAGCAAGGAAAGCATCCTGCCGAAGAGAAACAGCGACAAGCTGATCGCAAGAAAGAAGGACTGGGACCCGAAGAAGTACGG | |
| | AGGATTCGACAGCCCGACAGTCGCATACAGCGTCCTGGTCGTCGCAAAGGTCGAAAAGGGAAAGAGCAAGAAGCTGAAGAGCGTCA | |
| | AGGAACTGCTGGGAATCACAATCATGGAAAGAAGCAGCTTCGAAAAGAACCCGATCGACTTCCTGGAAGCAAAGGGATACAAGGAA | |
| | GTCAAGAAGGACCTGATCATCAAGCTGCCGAAGTACAGCCTGTTCGAACTGGAAAACGGAAGAAAGAGAATGCTGGCAAGCGCAGG | |
| | AGAACTGCAGAAGGGAAACGAACTGGCACTGCCGAGCAAGTACGTCAACTTCCTGTACCTGGCAAGCCACTACGAAAAGCTGAAGG | |
| | GAAGCCCGGAAGACAACGAACAGAAGCAGCTGTTCGTCGAACAGCACAAGCACTACCTGGACGAAATCATCGAACAGATCAGCGAA | |
| | TTCAGCAAGAGAGTCATCCTGGCAGACGCAAACCTGGACAAGGTCCTGAGCGCATACAACAAGCACAGAGACAAGCCGATCAGAGA | |
| | ACAGGCAGAAAACATCATCCACCTGTTCACACTGACAAACCTGGGAGCACCGGCAGCATTCAAGTACTTCGACACAACAATCGACA | |
| | GAAAGAGATACACAAGCACAAAGGAAGTCCTGGACGCAACACTGATCCACCAGAGCATCACAGGACTGTACGAAACAAGAATCGAC | |
| | CTGAGCCAGCTGGGAGGAGACGGAGGAGGAAGCCCGAAGAAGAAGAGAAAGGTCAGCGAAAGCGCAACACCGGAAAGCGTCAGCGG | |
| | ATGGAGACTGTTCAAGAAGATCAGCTAGCTAGCCATCACATTTAAAAGCATCTCAGCCTACCATGAGAATAAGAGAAAGAAAATGA | |
| | AGATCAATAGCTTATTCATCTCTTTTTCTTTTTCGTTGGTGTAAAGCCAACACCCTGTCTAAAAAACATAAATTTCTTTAATCATT | |
| | TTGCCTCTTTTCTCTGTGCTTCAATTAATAAAAAATGGAAAGAACCTCGAG | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 386 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS1 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSLAAK | |
| | RSRTT | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 387 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS2 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSQAAK | |
| | RSRTT | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 388 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS3 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSPAPA | |
| | KRERTT | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 389 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS4 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSQAAK | |
| | RPRTT | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 390 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS5 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSRAAK | |
| | RPRTT | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 391 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS6 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSAAAK | |
| | RSWSMAA | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 392 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS7 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSAAAK | |
| | RVWSMAF | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 393 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS8 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSAAAK | |
| | RSWSMAF | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 394 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS9 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSAAAK | |
| | RKYFAA | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 395 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS10 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSRAAK | |
| | RKAFAA | |
| Amino acid | MDKKYSIGLDIGTNSVGWAVITDEYKVPSKKFKVLGNTDRHSIKKNLIGALLFDSGETAEATRLKRTARRRYTRRKNRICYLQEIF | 396 |
| sequence for | SNEMAKVDDSFFHRLEESFLVEEDKKHERHPIFGNIVDEVAYHEKYPTIYHLRKKLVDSTDKADLRLIYLALAHMIKFRGHFLIEG | |
| Cas9 with | DLNPDNSDVDKLFIQLVQTYNQLFEENPINASGVDAKAILSARLSKSRRLENLIAQLPGEKKNGLFGNLIALSLGLTPNFKSNFDL | |
| NLS11 | AEDAKLQLSKDTYDDDLDNLLAQIGDQYADLFLAAKNLSDAILLSDILRVNTEITKAPLSASMIKRYDEHHQDLTLLKALVRQQLP | |
| | EKYKEIFFDQSKNGYAGYIDGGASQEEFYKFIKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDKGASAQSFIERMTNFDKNLPNEKVLPKHSLLYE | |
| | YFTVYNELTKVKYVTEGMRKPAFLSGEQKKAIVDLLFKTNRKVTVKQLKEDYFKKIECFDSVEISGVEDRFNASLGTYHDLLKIIK | |
| | DKDFLDNEENEDILEDIVLTLTLFEDREMIEERLKTYAHLFDDKVMKQLKRRRYTGWGRLSRKLINGIRDKQSGKTILDFLKSDGF | |
| | ANRNFMQLIHDDSLTFKEDIQKAQVSGQGDSLHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELDINRLSDYDVDHIVPQSFLKDDSIDNKVLTRS | |
| | DKNRGKSDNVPSEEVVKKMKNYWRQLLNAKLITQRKFDNLTKAERGGLSELDKAGFIKRQLVETRQITKHVAQILDSRMNTKYDEN | |
| | DKLIREVKVITLKSKLVSDFRKDFQFYKVREINNYHHAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFATVRKVLSMPQVNIVKKTEVQTGGFSKESILPKRNSDK | |
| | LIARKKDWDPKKYGGFDSPTVAYSVLVVAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQHKHYLDEIIEQISEFSKRVILADANLDKV | |
| | LSAYNKHRDKPIREQAENIIHLFTLTNLGAPAAFKYFDTTIDRKRYTSTKEVLDATLIHQSITGLYETRIDLSQLGGDGGGSRAAK | |
| | RKYFAV | |

* = PS linkage;
‘m’ = 2′-O-Me nucleotide

What is claimed is:

1. A method of treating amyloidosis associated with TTR (ATTR), comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
thereby treating ATTR.

2. A method of reducing TTR serum concentration, comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
thereby reducing TTR serum concentration.

3. A method for reducing or preventing the accumulation of amyloids or amyloid fibrils comprising TTR in a subject, comprising administering a corticosteroid and a composition to a subject in need thereof, wherein the composition comprises (i) an RNA-guided DNA binding agent or a nucleic acid encoding an RNA-guided DNA binding agent and (ii) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
thereby reducing accumulation of amyloids or amyloid fibrils.

4. A composition comprising a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

5. A composition comprising a vector encoding a guide RNA, wherein the guide RNA comprises:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82,
for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

6. A composition comprising:
(i) a guide RNA comprising:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82, and
(ii) an mRNA that encodes an RNA-guided DNA binding agent, wherein:
a. the open reading frame comprises a sequence with at least 95% identity to SEQ ID NO: 311;
b. the open reading frame has at least 95% identity to SEQ ID NO: 311 over at least its first 30, 50, 70, 100, 150, 200, 250, or 300 nucleotides;
c. the open reading frame consists of a set of codons of which at least 75% of the codons are codons listed in Table 1;
d. the open reading frame has an adenine content ranging from its minimum adenine content to 150% of the minimum adenine content; and/or
e. the open reading frame has an adenine dinucleotide content ranging from its minimum adenine dinucleotide content to 150% of the minimum adenine dinucleotide content;
for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

7. A composition comprising:
(i) a vector encoding a guide RNA, wherein the guide RNA comprises:
a. a guide sequence selected from SEQ ID NOs: 5-82;
b. at least 17, 18, 19, or 20 contiguous nucleotides of a sequence selected from SEQ ID NOs: 5-82; or
c. a guide sequence that is at least 99%, 98%, 97%, 96%, 95%, 94%, 93%, 92%, 91%, or 90% identical to a sequence selected from SEQ ID NOs: 5-82, and
(ii) an mRNA that encodes an RNA-guided DNA binding agent, wherein:
a. the open reading frame comprises a sequence with at least 95% identity to SEQ ID NO: 311;
b. the open reading frame has at least 95% identity to SEQ ID NO: 311 over at least its first 30, 50, 70, 100, 150, 200, 250, or 300 nucleotides;
c. the open reading frame consists of a set of codons of which at least 75% of the codons are codons listed in Table 1;
d. the open reading frame has an adenine content ranging from its minimum adenine content to 150% of the minimum adenine content; and/or
e. the open reading frame has an adenine dinucleotide content ranging from its minimum adenine dinucleotide content to 150% of the minimum adenine dinucleotide content;
for use in combination with a corticosteroid in a method of inducing a double-stranded break (DSB) within the TTR gene in a subject, modifying the TTR gene in a cell or subject, treating amyloidosis associated with TTR (ATTR) in a subject, reducing TTR serum concentration in a subject, and/or reducing or preventing the accumulation of amyloids or amyloid fibrils in a subject.

8. The composition for use or method of any one of claim 1-3 or 5-7, wherein the method comprises administering the composition by infusion for more than 30 minutes, e.g. more than 60 minutes or more than 120 minutes.

9. The composition or method of any one of claims 1-8, wherein the guide RNA comprises a guide sequence selected from SEQ ID NOs: 5-72, 74-78, and 80-82.

10. The composition or method of any one of the preceding claims, wherein the guide RNA comprises a guide sequence selected from SEQ ID NOs: 5, 6, 7, 8, 9, 12, 13, 14, 15, 16, 17, 22, 23, 27, 29, 30, 35, 36, 37, 38, 55, 61, 63, 65, 66, 68, or 69.

11. The composition of any one of claims 4-10, for use in inducing a double-stranded break (DSB) within the TTR gene in a cell or subject, modifying the TTR gene in a cell or subject, treating amyloidosis associated with TTR (ATTR) in a subject, or reducing TTR serum concentration in a subject, or reducing or preventing the accumulation of amyloids or amyloid fibrils in a subject.

12. The method or composition for use of any one of claims 1-11, wherein the corticosteroid is dexamethasone, betamethasone, prednisone, prednisolone, methylprednisolone, cortisone, hydrocortisone, triamcinolone, or ethamethasoneb.

13. The method or composition for use of any one of claims 1-12, wherein the corticosteroid is dexamethasone.

14. The method or composition for use of any one of claims 1-13, wherein the corticosteroid is administered before the composition.

15. The method or composition for use of any one of claims 1-14, wherein the corticosteroid is administered after the composition.

16. The method or composition for use of any one of claims 1-15, wherein the corticosteroid is administered simultaneously with the composition.

17. The method or composition for use of any one of claims 1-16, wherein the corticosteroid is administered about 5 minutes to within about 168 hours before the composition is administered.

18. The method or composition for use of any one of claims 1-17, wherein the corticosteroid is administered about 5 minutes to within about 168 hours after the composition is administered.

19. The method or composition for use of any one of claims 1-18, wherein the corticosteroid is administered 5 minutes, 10 minutes, 15 minutes, 30 minutes, 1 hour, 2 hours, 3 hours, 4 hours, 6 hours, 12 hours, 18 hours, 1 day, 1.5 days, 2 days, 3 days, 4 days, 5 days, 6 days, or one week before the composition is administered.

20. The method or composition for use of any one of claims 1-19, wherein at least two doses of the corticosteroid are administered before or after the administration of the composition.

21. The method or composition for use of any one of claims 1-20, wherein at least two doses of the corticosteroid and at least two doses of the composition are administered.

22. The method or composition for use of any one of claims 1-21, wherein the corticosteroid is administered to the subject at a dose of 0.75 mg to 20 mg, or at a dose of about 0.01-0.4 mg/kg, such as 0.1-0.35 mg/kg or 0.25-0.35 mg/kg.

23. The method or composition for use of any one of claims 1-22, wherein the corticosteroid is administered to the subject via an intravenous injection.

24. The method or composition for use of any one of claims 1-23, wherein the corticosteroid is administered to the subject orally, optionally wherein the corticosteroid is administered to the subject orally before the composition is administered to the subject by intravenous injection.

25. The method or composition for use of claim 24, wherein the corticosteroid is dexamethasone, and the dexamethasone is administered to the subject orally in the amount of 20 mg 6 to 12 hour before the composition is administered to the subject, or the dexamethasone is administered to the subject intravenously in the amount of 20 mg for 30 minutes 6 to 12 hour before the composition is administered to the subject.

26. The method or composition for use of any one of claims 1-25, wherein the composition is administered by infusion for about 60 minutes, about 90 minutes, about 120 minutes, about 150 minutes, about 180 minutes, or about 240 minutes.

27. The method or composition for use of any one of claims 1-26, wherein the corticosteroid is dexamethasone.

28. The method or composition for use of any one of claims 1-27, wherein the method further comprises administering an infusion prophylaxis, wherein the infusion prophylaxis comprises one or more of acetaminophen, an H1 blocker, or an H2 blocker, optionally wherein the one or more of the acetaminophen, H1 blocker, or H2 blocker are concurrently administered with the corticosteroid and/or before the composition.

29. The method or composition for use of claim 28, wherein each of the acetaminophen, H1 blocker, and H2 blocker are administered.

30. The method or composition for use of claim 28 or 29, wherein the H1 blocker and/or the H2 blocker are administered orally.

31. The method or composition for use of any one of claims 28-30, wherein the infusion prophylaxis comprises an intravenous corticosteroid (such as dexamethasone 8-12 mg, or 10 mg or equivalent) and acetaminophen (such as oral acetaminophen 500 mg).

32. The method or composition for use of any one of claims 28-31, wherein the infusion prophylaxis is administered as a required premedication prior to administering a guide RNA-containing composition, e.g. an LNP composition.

33. The method or composition for use of any one of claims 28-32, wherein H1 blocker is diphenhydramine.

34. The method or composition for use of any one of claims 28-33, wherein the H2 blocker is ranitidine.

35. The method or composition for use of any one of claims 1-35, wherein a first dose of the corticosteroid is administered at about 8-24 hours before the composition is administered and a second dose of the corticosteroid is administered at about 1-2 hours before the composition is administered.

36. The method or composition for use of claim 35, wherein the method further comprises administering one or more of acetaminophen, an H1 blocker, or an H2 blocker, optionally wherein the one or more of the acetaminophen, H1 blocker, or H2 blocker are concurrently administered with the second dose of the corticosteroid.

37. The method or composition for use of any one of claims 1-36, wherein a first dose of the corticosteroid is administered orally at about 8-24 hours before the composition is administered and a second dose of the corticosteroid is administered intravenously at about 1-2 hours before the composition is administered.

38. The method or composition for use of any one of claims 1-37, wherein a first dose of the corticosteroid is administered orally at about 8-24 hours before the composition is administered and a second dose of the corticosteroid is administered intravenously concurrently with administration of acetaminophen, H1 blocker and H2 blocker at about 1-2 hours before the composition is administered.

39. The method or composition for use of any one of claims 1-38, wherein the corticosteroid is dexamethasone, and a first dose of dexamethasone in the amount of about 6-10 mg is administered to the subject orally at about 8-24 hours before the composition is administered to the subject, and a second dose of dexamethasone in the amount of about 8-12 mg is intravenously administered to the subject concurrently with oral administration of acetaminophen and intravenous administration of an H1 blocker and an H2 blocker, at about 1-2 hours before the composition is administered to the subject, optionally wherein the H1 blocker is diphenhydramine and the H2 blocker is ranitidine, and/or optionally wherein the subject is human.

40. The method or composition for use of any one of claims 1-39, wherein the corticosteroid is dexamethasone, and a first dose of dexamethasone in the amount of 8 mg is administered to the subject orally at about 8-24 hours before the composition is administered to the subject, and a second dose of dexamethasone in the amount of 10 mg is intravenously administered to the subject concurrently with oral administration of acetaminophen and intravenous administration of an H1 blocker and an H2 blocker, at about 1-2 hours before the composition is administered to the subject, optionally wherein the H1 blocker is diphenhydramine and the H2 blocker is ranitidine.

41. The method or composition for use of any one of claims 1-40, wherein the composition is administered in the amount of 3 mg/kg by infusion for about 1.5-6 hours; a first dose of the corticosteroid is administered orally at about 8-24 hours before infusion of the composition; and a second dose of the corticosteroid is administered intravenously at about 1-2 hours before infusion of the composition.

42. The method or composition for use of any one of claims 1-41, wherein administering the corticosteroid improves tolerability of the composition comprising the guide RNA.

43. The method or composition for use of any one of claims 1-42, wherein administering the corticosteroid reduces the incidence or severity of one or more of inflammation, nausea, vomiting, elevated ALT concentration in blood, hyperthermia, and/or hyperalgesia in response to the composition comprising the guide RNA.

44. The method or composition for use of any one of claims 1-43, wherein administering the corticosteroid reduces or inhibits production or activity of one or more interferons and/or inflammatory cytokines in response to the composition comprising the guide RNA.

45. The method or composition for use of any one of claims 1-44, wherein the composition reduces serum TTR levels.

46. The method or composition for use of claim 45, wherein the serum TTR levels are reduced by at least 50% as compared to serum TTR levels before administration of the composition.

47. The method or composition for use of any one of claims 1-46, wherein the composition results in editing of the TTR gene.

48. The method or composition for use of claim 47, wherein the editing is calculated as a percentage of the population that is edited (percent editing), optionally wherein the percent editing is between 30 and 99% of the population.

49. The method or composition for use of claims 1-48, wherein the composition reduces amyloid deposition in at least one tissue, optionally wherein the at least one tissue comprises one or more of stomach, colon, sciatic nerve, or dorsal root ganglion.

50. The method or composition for use of claims 1-49, wherein the composition is administered or delivered at least two times.

51. The method or composition for use of claim 50, wherein the administration or delivery occurs at an interval of 1, 2, 3, 4, 5, 6, 7, 8, 9, 10, 11, 12, 13, 14, or 15 days, 1, 2, 3, 4, 5, 6, 7, 8, 9, 10, 11, 12, 13, 14, or 15 weeks, 1, 2, 3, 4, 5, 6, 7, 8, 9, 10, 11, 12, 13, 14, or 15 months.

52. The method or composition of any one of claims 1-51, wherein the guide sequence is selected from SEQ ID NOs: 5-82.

53. The method or composition of any one of claims 1-52, wherein the guide RNA is at least partially complementary to a target sequence present in the human TTR gene.

54. The method or composition of claim 53, wherein the target sequence is in exon 1, 2, 3, or 4 of the human TTR gene.

55. The method or composition of any one of claims 1-54, wherein the guide sequence is complementary to a first target sequence in the positive strand of the TTR gene, and wherein the composition further comprises a second guide sequence that is complementary to a second target sequence in the negative strand of the TTR gene.

56. The method or composition of any one of claims 1-55, wherein the guide RNA is a single guide (sgRNA).

57. The method or composition of claim 56, wherein the sgRNA comprises any one of the guide sequences of SEQ ID NOs: 5-82 and nucleotides 21-100 of SEQ ID NO: 3.

58. The method or composition of claim 56, wherein the sgRNA comprises a guide sequence that is at least 99%, 98%, 97%, 96%, 95%, 94%, 93%, 92%, 91%, or 90% identical to a sequence selected from SEQ ID Nos: 87-124.

59. The method or composition of claim 58, wherein the sgRNA comprises a sequence selected from SEQ ID Nos: 87-124.

60. The method or composition of any one of claims 1-59, wherein the guide RNA comprises at least one modification.

61. The method or composition of claim 60, wherein the at least one modification includes a 2′-O-methyl (2′-O-Me) modified nucleotide, a phosphorothioate (PS) bond between nucleotides, or a 2′-fluoro (2′-F) modified nucleotide.

62. The method or composition of any one of claims 60-61, wherein the at least one modification includes PS bonds between the first four nucleotides, PS bonds between the last four nucleotides, 2′-O-Me modified nucleotides at the first three nucleotides at the 5′ end, and/or 2′-O-Me modified nucleotides at the last three nucleotides at the 3′ end.

63. The method or composition of any one of claims 60-62, wherein the guide RNA comprises the modified nucleotides of SEQ ID NO: 3.

64. The method or composition of any one of claims 1-63, wherein the guide RNA is associated with a lipid nanoparticle (LNP).

65. The method or composition of claim 64, wherein the LNP comprises an ionizable lipid.

66. The method or composition of any one of claims 64-65, wherein the LNP comprises a biodegradable ionizable lipid.

67. The method or composition of any one of claims 64-65, wherein the LNP comprises an amine lipid, e.g., a CCD lipid.

68. The method or composition of any one of claims 64-66, wherein the LNP comprises a helper lipid.

69. The method or composition of any one of claims 64-67, wherein the LNP comprises a stealth lipid, optionally wherein:
(i) the LNP comprises a lipid component and the lipid component comprises: about 50-60 mol-% amine lipid such as Lipid A, about 8-10 mol-% neutral lipid; and about 2.5-4 mol-% stealth lipid (e.g., a PEG lipid), wherein the remainder of the lipid component is helper lipid, and wherein the N/P ratio of the LNP composition is about 6;
(ii) the LNP comprises about 50-60 mol-% amine lipid such as Lipid A; about 27-39.5 mol-% helper lipid; about 8-10 mol-% neutral lipid; and about 2.5-4 mol-% stealth lipid (e.g., a PEG lipid), wherein the N/P ratio of the LNP composition is about 5-7 (e.g., about 6);
(iii) the LNP comprises a lipid component and the lipid component comprises: about 50-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 3-10;
(iv) the LNP comprises a lipid component and the lipid component comprises: about 40-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the LNP composition is about 6;
(v) the LNP comprises a lipid component and the lipid component comprises: about 50-60 mol-% amine lipid such as Lipid A; about 5-15 mol-% neutral lipid; and about 1.5-10 mol-% Stealth lipid (e.g., a PEG lipid), wherein the remainder of the lipid component is helper lipid, and wherein the N/P ratio of the LNP composition is about 6;
(vi) the LNP comprises a lipid component and the lipid component comprises: about 40-60 mol-% amine lipid such as Lipid A; about 0-10 mol-% neutral lipid; and about 1.5-10 mol-% Stealth lipid (e.g., a PEG lipid), wherein the remainder of the lipid component is helper lipid, and wherein the N/P ratio of the LNP composition is about 3-10;
(vii) the LNP comprises a lipid component and the lipid component comprises: about 40-60 mol-% amine lipid such as Lipid A; less than about 1 mol-% neutral lipid; and about 1.5-10 mol-% Stealth lipid (e.g., a PEG lipid), wherein the remainder of the lipid component is helper lipid, and wherein the N/P ratio of the LNP composition is about 3-10;
(viii) the LNP comprises a lipid component and the lipid component comprises: about 40-60 mol-% amine lipid such as Lipid A; and about 1.5-10 mol-% Stealth lipid (e.g., a PEG lipid), wherein the remainder of the lipid component is helper lipid, wherein the N/P ratio of the LNP composition is about 3-10, and wherein the LNP composition is essentially free of or free of neutral phospholipid; or
(ix) the LNP comprises a lipid component and the lipid component comprises: about 50-60 mol-% amine lipid such as Lipid A; about 8-10 mol-% neutral lipid; and about 2.5-4 mol-% Stealth lipid (e.g., a PEG lipid), wherein the remainder of the lipid component is helper lipid, and wherein the N/P ratio of the LNP composition is about 3-7.

70. The method or composition of any one of claims 64-69, wherein the LNP comprises a neutral lipid.

71. The method or composition of any one of claims 64-70, wherein the LNP comprises a lipid component and the lipid component comprises: about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the LNP composition is about 6.

72. The method or composition of any one of claims 64-71, wherein the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

73. The method or composition of any one of claims 1-72, wherein the composition further comprises an RNA-guided DNA binding agent.

74. The method or composition of any one of claims 1-72, wherein the composition further comprises a polynucleotide that encodes an RNA-guided DNA binding agent.

75. The method or composition of claim 74, wherein the polynucleotide is an mRNA.

76. The method or composition of any one of claims 73-75, wherein the RNA-guided DNA binding agent is a Cas cleavase.

77. The method or composition of any one of claims 74-76, wherein the polynucleotide comprises an open reading frame encoding an RNA-guided DNA binding agent, wherein:
a. the open reading frame comprises a sequence with at least 95% identity to SEQ ID NO: 311;
b. the open reading frame has at least 95% identity to SEQ ID NO: 311 over at least its first 30, 50, 70, 100, 150, 200, 250, or 300 nucleotides;
c. the open reading frame consists of a set of codons of which at least 75% of the codons are codons listed in Table 4;
d. the open reading frame has an adenine content ranging from its minimum adenine content to 150% of the minimum adenine content; and/or
e. the open reading frame has an adenine dinucleotide content ranging from its minimum adenine dinucleotide content to 150% of the minimum adenine dinucleotide content.

78. The composition or method of any one of claims 74-77, wherein the polynucleotide comprises a 5′ UTR with at least 90% identity to any one of SEQ ID NOs: 232, 234, 236, 238, 241, or 275-277; and/or a 3′ UTR with at least 90% identity to any one of SEQ ID NOs: 233, 235, 237, 239, or 240.

79. The composition or method of any of claims 74-78, wherein the polynucleotide is an mRNA and at least 10% of the uridine in the mRNA is substituted with a modified uridine.

80. The method or composition of any one of claims 73-79, wherein the RNA-guided DNA binding agent is modified.

81. The method or composition of claim 80, wherein the modified RNA-guided DNA binding agent comprises a nuclear localization signal (NLS).

82. The method or composition of any one of claims 1-81, wherein the composition is a pharmaceutical formulation and further comprises a pharmaceutically acceptable carrier.

83. The method or composition for use of any one of claims 1-82, wherein the composition reduces or prevents amyloids or amyloid fibrils comprising TTR.

84. The method or composition for use of any one of claims 1-83, wherein non-homologous ending joining (NHEJ) leads to a mutation during repair of a DSB in the TTR gene.

85. The method or composition of any one of claims 1-84, wherein the sequence of the guide RNA is:
a) SEQ ID NO: 92 or 104;
b) SEQ ID NO: 87, 89, 96, or 113;
c) SEQ ID NO: 100, 102, 106, 111, or 112; or
d) SEQ ID NO: 88, 90, 91, 93, 94, 95, 97, 101, 103, 108, or 109,
optionally wherein the guide RNA does not produce indels at off-target site(s) that occur in a protein coding region in the genome of primary human hepatocytes.

86. The method or composition for use of any one of claims 1-85, wherein administering the composition reduces levels of TTR in the subject, optionally wherein the levels of TTR are reduced by at least 50%.

87. The method or composition for use of claim 86, wherein the levels of TTR are measured in serum, plasma, blood, cerebral spinal fluid, or sputum, or liver, choroid plexus, and/or retina, optionally wherein the levels of TTR are measured via enzyme-linked immunosorbent assay (ELISA).

88. The method or composition for use of claims 1-87, wherein the subject has ATTR, familial amyloid polyneuropathy or familial amyloid cardiomyopathy.

89. The method or composition for use of claims 1-88, wherein the subject is human.

90. The method or composition for use of claims 1-89, wherein the subject is tested for specific mutations in the TTR gene before administering the composition or formulation.

91. Use of a composition or formulation of any of claims 1-90 for the preparation of a medicament for treating a human subject having ATTR.
